(19) United States
(12) Patent Application Publication
(10) Pub. No.: US 2026/0028353 A1
(43) Pub. Date: Jan. 29, 2026

(54) MACROCYCLIC RAS INHIBITORS

(71) Applicant: Revolution Medicines, Inc., Redwood City, CA (US)

(72) Inventors: Elena S. KOLTUN, Foster City, CA (US); John E. Knox, Emerald Hills, CA (US); Nguyen Hong Quang Luu, San Mateo, CA (US); Dylan E. Parsons, San Carlos, CA (US); Severin Thompson, Belmont, CA (US); Aidan Tomlinson, San Francisco, CA (US); Mark Tye, Redwood City, CA (US); Kingsley Y. Wu, Alhambra, CA (US); Andreas Buckl, Basel (CH); G. Leslie Burnett, Redwood City, CA (US); James Cregg, Belmont, CA (US); Ann V. Edwards, San Mateo, CA (US); Adrian L. Gill, Atherton, CA (US)

(21) Appl. No.: 19/350,302

(22) Filed: Oct. 6, 2025

Related U.S. Application Data
(63) Continuation of application No. PCT/US2024/023272, filed on Apr. 5, 2024.
(60) Provisional application No. 63/457,850, filed on Apr. 7, 2023.
(60) Provisional application No. 63/611,585, filed on Dec. 18, 2023.
(60) Provisional application No. 63/624,178, filed on Jan. 23, 2024.

(51) Int. Cl. C07D 498/22; A61K 31/496; A61K 31/504; A61K 31/5377; A61K 31/5383; A61K 31/55; C07D 498/18; C07D 513/22; C07D 519/00

(52) U.S. Cl. CPC C07D 498/22; A61K 31/496; A61K 31/504; A61K 31/5377; A61K 31/5383; A61K 31/55; C07D 498/18; C07D 513/22; C07D 519/00

(57) ABSTRACT

The disclosure features macrocyclic compounds, and pharmaceutical compositions and protein complexes thereof, capable of inhibiting Ras proteins, and their uses in the treatment of cancers.

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations.

### SUMMARY

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in an aspect, the disclosure features a compound having the structure of Formula Ia or Formula Ib:

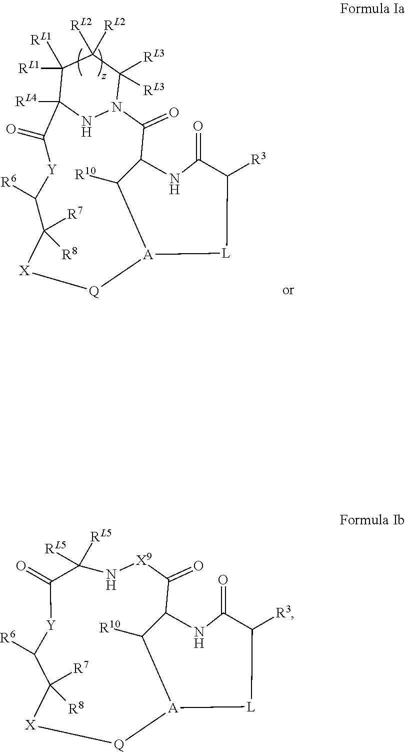

or a pharmaceutically acceptable salt thereof, wherein:
Q is an optionally substituted 7- to 12-membered bicyclic arylene, an optionally substituted 7- to 12-membered bicyclic heteroarylene, an optionally substituted 7- to 12-membered bicyclic heterocyclylene, wherein a first ring in Q is bonded to X, and a second ring in Q is bonded to A;
X is a bond; a straight chain C₁-C₃ alkylene optionally substituted with 1 to 3 substituents independently selected from fluoro, —CN, —C₁-C₃ alkyl, and —O—C₁-C₃ alkyl; —O—; —S(O)₀₋₂—; *—CH₂—O—; *—CH₂—S(O)₀₋₂—; *—O—CH₂—; or *—CH₂—S(O)₀₋₂—, wherein “*” represents a portion of X bound to —C(R⁷)(R⁸)—;
Y is —O—, —NH— or —N(C₁-C₃ alkyl)-;
A is optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
L is a linker;
R³ is optionally substituted C₁-C₆ alkyl, optionally substituted C₃-C₆ cycloalkyl, optionally substituted C₆ aryl, or optionally substituted 3- to 7-membered heterocyclyl;
R¹⁰ is hydrogen, halogen, optionally substituted C₁-C₃ alkyl, or C₁-C₃ optionally substituted heteroalkyl;
R⁷ is hydrogen, halogen, or optionally substituted C₁-C₃ alkyl;
R⁸ is hydrogen, halogen, —OH, —CN, —O-(optionally substituted C₁-C₃ alkyl), optionally substituted C₁-C₃ alkyl, optionally substituted C₂-C₆ alkenyl, optionally substituted C₂-C₆ alkynyl, optionally substituted C₆-C₁₀ aryl, optionally substituted 4- to 8-membered heteroaryl, optionally substituted C₃-C₆ cycloalkyl, or optionally substituted 3- to 7-membered heterocyclyl; or
R⁷ and R⁸ together form ═CH₂, an optionally substituted C₃-C₆ cycloalkyl, or a 3- to 7-membered saturated heterocyclyl; or
R⁸ and a ring atom in Q, the carbon atom to which R⁷ is bound, and X to form a 4- to 9-membered saturated or unsaturated heterocyclyl that is fused to Q;
R⁶ is hydrogen or —CH₃;
each R⁵ is independently halogen, optionally substituted C₁-C₃ alkyl, or optionally substituted C₁-C₃ haloalkyl;
p is 0, 1, 2, or 3;
z is 0, 1, or 2;
X⁹ is —NR^{L6}—, —C(O)— or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In an aspect, the invention features a compound having the structure of Formula IIa or Formula IIb:

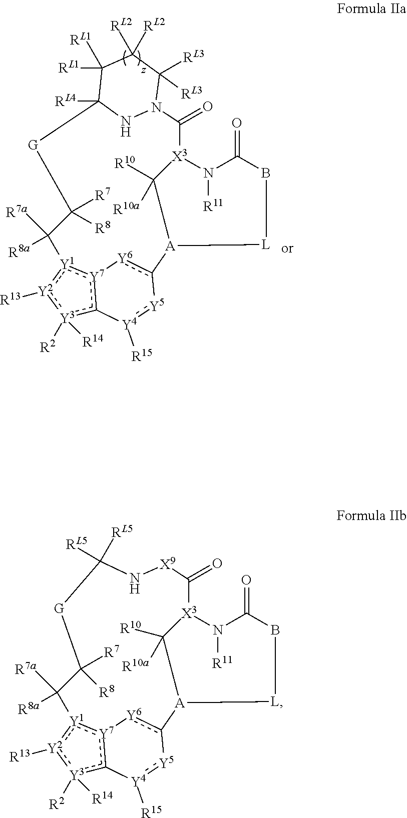

or a pharmaceutically acceptable salt thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CR^{x}, CH, CH₂, or N;
Y⁶ is CR^{z}, C(O), CH, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{z} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl;
R²¹ is hydrogen or optionally substituted C₁-C₃ alkyl;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In an aspect, the invention features a compound having the structure of Formula IIa-1:

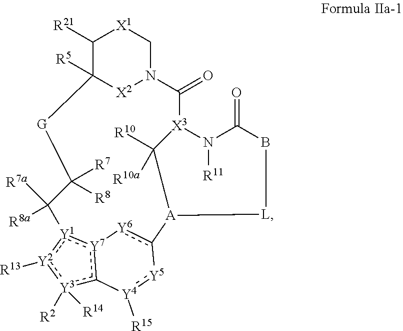

or a pharmaceutically acceptable salt thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CR^{x}, CH, CH₂, or N;
Y⁶ is CR^{z}, C(O), CH, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{z} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl; and
R²¹ is hydrogen or optionally substituted C₁-C₃ alkyl.

In an aspect, the invention features a compound having the structure of Formula IIIa or Formula IIIb:

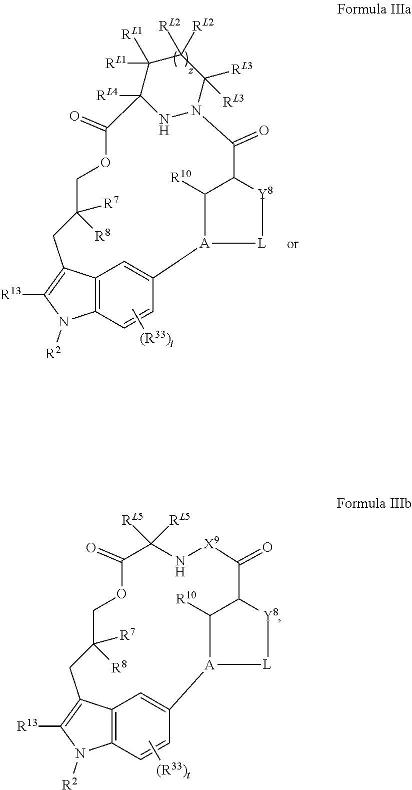

or a pharmaceutically acceptable salt thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is

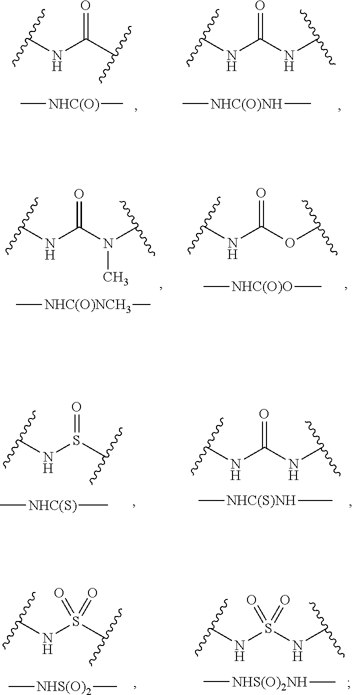

L is a linker;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl;
R⁷ and R⁸ are each, independently, selected from F or CH₃, or R⁷ and R⁸ combine with the atoms to which they are attached to make a 3-membered cycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
t is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In an aspect, the invention features a compound having the structure of Formula IIIa-1:

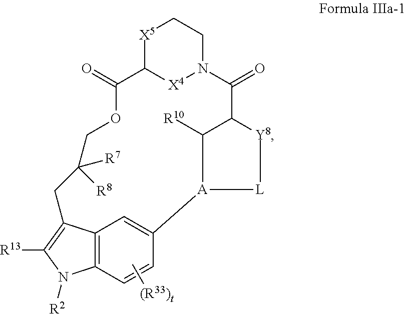

or a pharmaceutically acceptable salt thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is

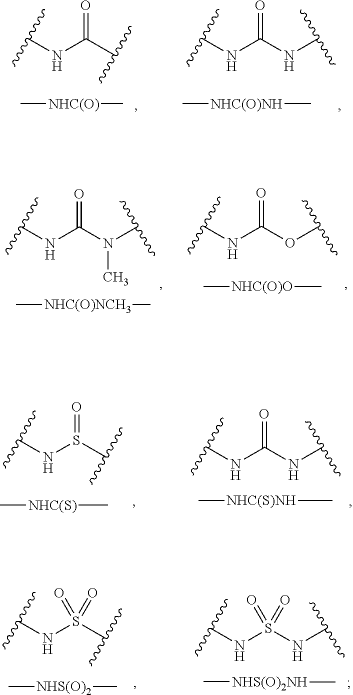

L is a linker;
X⁴ and X⁵ are each, independently, CH₂, CH(CH₃) or NH;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl;
R⁷ and R⁸ are each, independently, selected from F or CH₃, or R⁷ and R⁸ combine with the atoms to which they are attached to make a 3-membered cycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In an aspect, the invention features a compound having the structure of Formula IVa or Formula IVb:

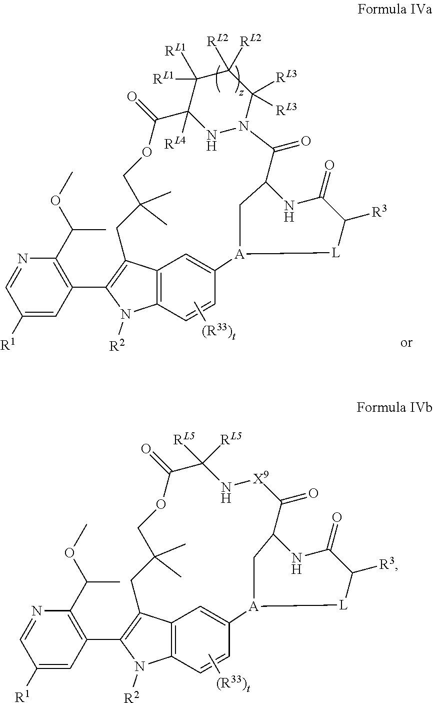

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In an aspect, the invention features a compound having the structure of Formula Va or Formula Vb:

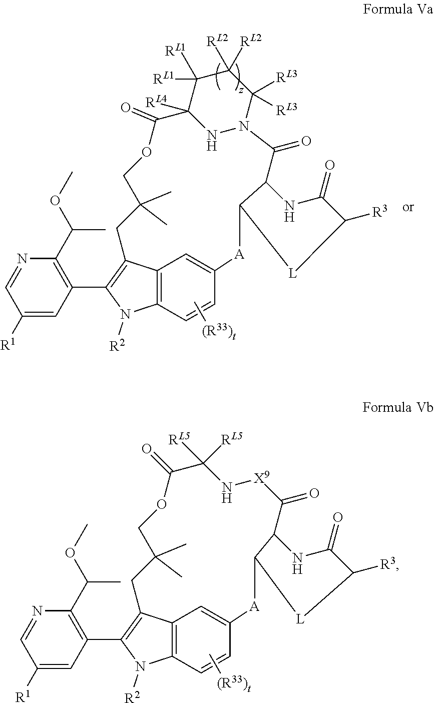

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In an aspect, the invention features a compound having the structure of Formula VIIa or Formula VIIb:

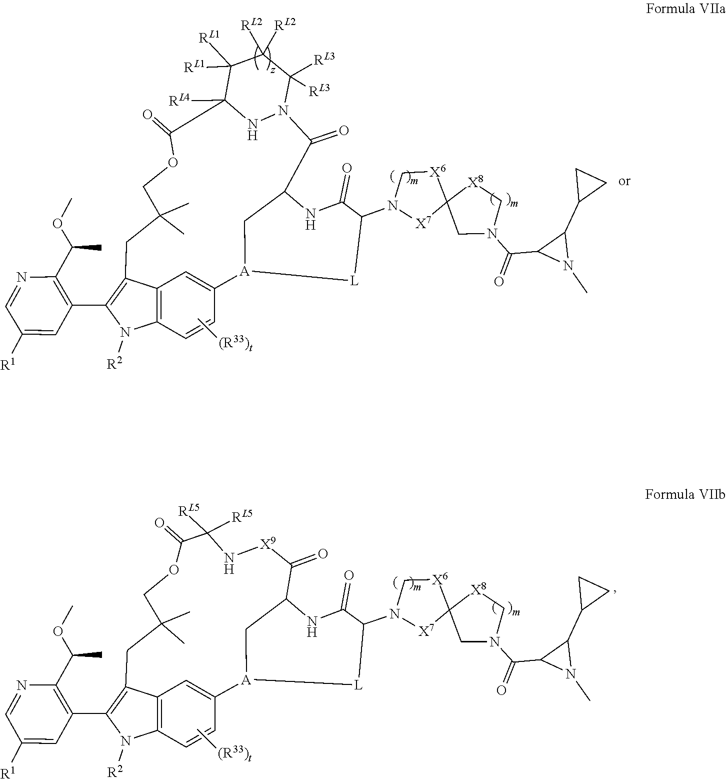

or a pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
X⁶, X⁷, and X⁸ are each independently selected from CH₂, CHF, CF₂, C═O, or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, optionally substituted C₁-C₆ heteroalkyl, or optionally substituted 3- to 10-membered heterocycloalkyl;
R² is optionally substituted C₁-C₆ alkyl;
R³ is optionally substituted C₁-C₆ alkyl, optionally substituted C₁-C₆ heteroalkyl, optionally substituted 3- to 6-membered cycloalkyl, or optionally substituted heterocycloalkyl, and wherein each hydrogen is independently, optionally, isotopically enriched for deuterium;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In an aspect, the invention features a compound having the structure of Formula XI:

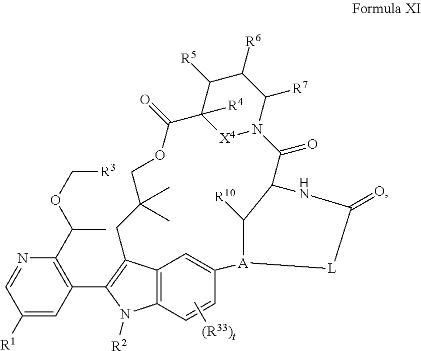

or pharmaceutically acceptable salt thereof, wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ alkenyl, optionally substituted C₁-C₆ alkynyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is —OR¹¹ or —NR¹²R¹³;
R¹¹, R¹², and R¹³ are each, independently, optionally substituted C₁-C₆ alkyl, optionally substituted C₁-C₆ heteroalkyl, or R¹² and R¹³ combine to form an optionally substituted 3- to 10-membered heterocycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In an aspect, the invention features a compound, or a pharmaceutically acceptable salt thereof, of Table 1.

Also provided are pharmaceutical compositions including a compound, or a pharmaceutically acceptable salt thereof, of any of the above aspects and embodiments, and a pharmaceutically acceptable excipient.

Also provided is a method of treating cancer in a subject in need thereof, the method comprising administering to the subject a therapeutically effective amount of a compound of the present invention, or a pharmaceutically acceptable salt thereof.

In some embodiments, a method is provided of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, for example, a compound of Table 1, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as 150 ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

Non-limiting examples of moieties that may contain one or more deuterium substitutions in compounds of the present invention, where any position “R” may be deuterium (D), include

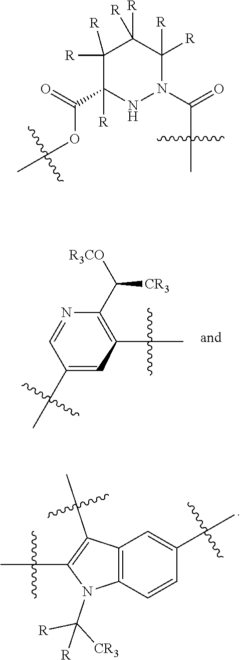

Additional examples include moieties such as

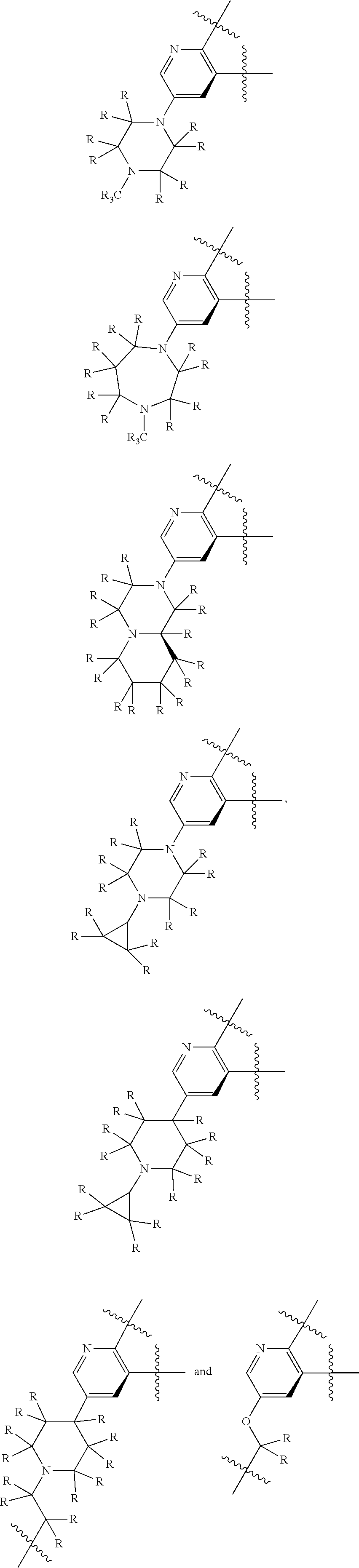

and deuteration of similar moieties, e.g., in compounds of Formula I, II, III, IV, V, and VI, and subformulae thereof). Moreover, deuteration of available positions in any A moiety of compounds of the Formulas described herein is also contemplated, such as

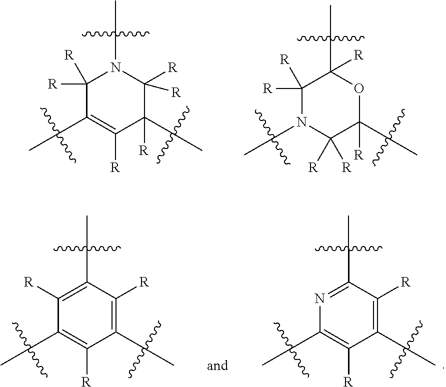

Further, deuterium substitution may also take place in compounds of the present invention at the linker position, such as

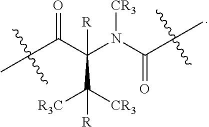

Further, deuterium substitution may also take place in compounds of the present invention at the linker position, such as

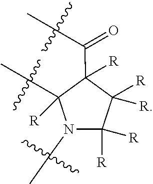

Further, deuterium substitution may also take place in compounds of the present invention at the linker position, such as

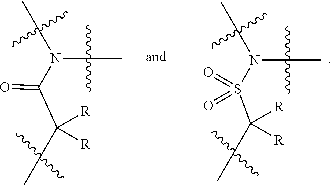

In a further embodiment, silylation substitution is also contemplated, such as in the linker as follows:

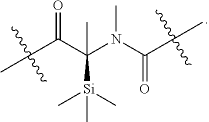 [SMILES: *C(=O)N(C)[C@@](C)(C(*)=O)[Si](C)(C)C]

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4- to 8-membered saturated or unsaturated heterocycloalkyl (e.g., pyridyl); 3- to 8-membered saturated or unsaturated cycloalkyl (e.g., cyclopropyl, cyclobutyl, or

cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R⁰)—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(C H₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NH R^{●}, —(CH₂)₀₋₂NR^{●}2, —NO₂, —SiR^{●}3, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently

halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

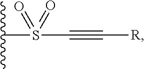

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(Co alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

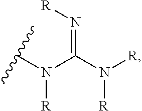

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., one portion of a macrocycle) to a second moiety (e.g., a second portion of the same macrocycle). In some embodiments, the linker results in a compound capable of achieving an IC50 of 2 μM or less in the Ras-RAF disruption assay protocol provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.
   This assay may be used to assess selectivity as well. In some embodiments, a compound of the present invention is selective for one or more particular Ras mutants over other Ras mutants or wild-type compared to what is known in the art. For example, a compound of Formula Ia and subformulae thereof may be more selective for K-Ras G12C or K-Ras G13C compared to K-Ras wild-type. For example, a compound of Formula IIa-1 and Formula IIa-2 may be more selective for K-Ras G12C, K-Ras G13C, K-Ras G12D or K-Ras G12V compared to K-Ras wild-type. For example, a compound of Formula IIIa-1 and Formula IIIa-2 may be more selective for K-Ras G12D or K-Ras G12V compared to K-Ras wild-type. For example, a compound of Formula IVa and subformulae thereof may be more selective for K-Ras G12C or K-Ras G13C compared to K-Ras wild-type. For example, a compound of Formula VIa and subformulae thereof may be more selective for K-Ras G12D compared to K-Ras wild-type. For example, a compound of Formula VIIa and subformulae thereof may be more selective for K-Ras G12D compared to K-Ras wild-type.

Without being bound by theory, the inventors postulate that non-covalent interactions of “L” and the chaperone protein (e.g., cyclophilin A) may contribute to the inhibition of Ras activity. For example, van der Waals, hydrophobic, hydrophilic and hydrogen bond interactions, and combinations thereof, may contribute to the ability of the compounds of the present invention to form complexes and act as Ras inhibitors. The inventors also postulate that “L” also imparts structural rigidity to the compounds, which may optimize these non-covalent interactions, thereby contributing to the inhibition of Ras activity.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

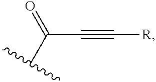

wherein R is any any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that covalent, non-covalent or combinations of covalent and non-covalent interactions of a compound of the present invention with Ras and the chaperone protein (e.g., cyclophilin A) may contribute to the inhibition of Ras activity. In some embodiments, a compound of the present invention forms a covalent adduct with a side chain of a Ras protein (e.g., a side chain of the histidine at position 61 of a mutant Ras protein). Covalent adducts may also be formed with other side chains of Ras. In addition, or alternatively, non-covalent interactions may be at play: for example, van der Waals, hydrophobic, hydrophilic and hydrogen bond interactions, and combinations thereof, may contribute to the ability of the compounds of the present invention to form complexes and act as Ras inhibitors.

Accordingly, a variety of Ras proteins may be inhibited by a compound of the present invention (e.g., K-Ras, N-Ras, H-Ras, and mutants thereof at positions 12, 13 and 61, such as G12C, G12D, G12V, G12S, G13C, G13D, Q61H, Q61K, Q61R and Q61L, and others described herein, or a combination thereof).

Methods of determining covalent adduct formation are known in the art. One method of determining covalent adduct formation is to perform a “cross-linking” assay, such as under these conditions.
Note—the following protocol describes a procedure for monitoring cross-linking of K-Ras G12C (GMP-PNP) to a compound of the invention. This protocol may also be executed substituting other Ras proteins or nucleotides.
The purpose of this biochemical assay is to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME (if studying a cysteine Ras mutant, such as K-Ras G12C or G13C), 5 μM cyclophilin A and 2 μM test compound, a 5 μM stock of GMP-PNP-loaded K-Ras (1-169) G12C is diluted 10-fold to yield a final concentration of 0.5 μM; with final sample volume being 100 μL.
The sample is incubated at 25° C. for a time period of up to 24 hours prior to quenching by the addition of 10 μL of 5% Formic Acid. Quenched samples are centrifuged at 15000 rpm for 15 minutes in a benchtop centrifuge before injecting a 10 μL aliquot onto a reverse phase C4 column and eluting into the mass spectrometer with an increasing acetonitrile gradient in the mobile phase. Analysis of raw data may be carried out using Waters MassLynx MS software, with % bound calculated from the deconvoluted protein peaks for labeled and unlabeled K-Ras. Accordingly, provided herein is a compound having the structure of Formula Ia or Formula Ib:

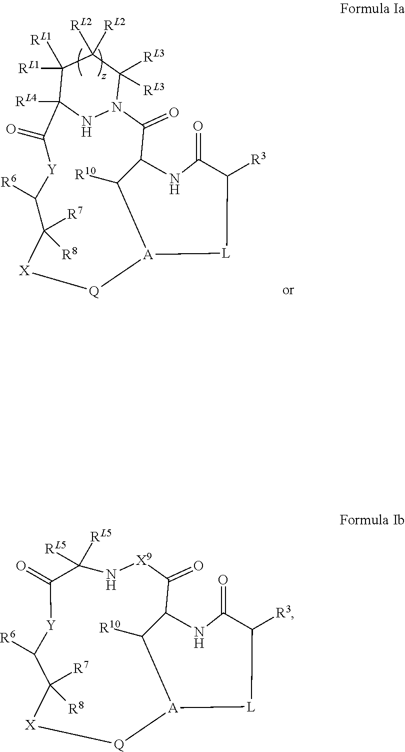

or pharmaceutically acceptable salt, an enantiomer, a stereoisomer, or a tautomer thereof, wherein:
Q is an optionally substituted 7- to 12-membered bicyclic arylene, an optionally substituted 7- to 12-membered bicyclic heteroarylene, an optionally substituted 7- to 12-membered bicyclic heterocyclylene, wherein a first ring in Q is bonded to X, and a second ring in Q is bonded to A;
X is a bond; a straight chain C₁-C₃ alkylene optionally substituted with 1 to 3 substituents independently selected from fluoro, —CN, —C₁-C₃ alkyl, and —O—C₁-C₃ alkyl; —O—; —S(O)₀₋₂—; *—CH₂—O—; *—CH₂—S(O)₀₋₂—; *—O—CH₂—; or *—CH₂—S(O)₀₋₂—, wherein “*” represents a portion of X bound to —C(R⁷)(R⁸)—;
Y is —O—, —NH— or —N(C₁-C₃ alkyl)-;
A is optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
L is a linker;
R³ is optionally substituted C₁-C₆ alkyl, optionally substituted C₃-C₆ cycloalkyl, optionally substituted C₆ aryl, or optionally substituted 3- to 7-membered heterocyclyl;
R¹⁰ is hydrogen, halogen, optionally substituted C₁-C₃ alkyl, or C₁-C₃ optionally substituted heteroalkyl;
R⁷ is hydrogen, halogen, or optionally substituted C₁-C₃ alkyl;
R⁸ is hydrogen, halogen, —OH, —CN, —O-(optionally substituted C₁-C₃ alkyl), optionally substituted C₁-C₃ alkyl, optionally substituted C₂-C₆ alkenyl, optionally substituted C₂-C₆ alkynyl, optionally substituted C₆-C₁₀ aryl, optionally substituted 4- to 8-membered heteroaryl, optionally substituted C₃-C₆ cycloalkyl, or optionally substituted 3- to 7-membered heterocyclyl; or
R⁷ and R⁸ together form ═CH₂, an optionally substituted C₃-C₆ cycloalkyl, or a 3- to 7-membered saturated heterocyclyl; or
R⁸ and a ring atom in Q, the carbon atom to which R⁷ is bound, and X to form a 4- to 9-membered saturated or unsaturated heterocyclyl that is fused to Q;
R⁶ is hydrogen or —CH₃;
each R⁵ is independently halogen, optionally substituted C₁-C₃ alkyl, or optionally substituted C₁-C₃ haloalkyl;
p is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In some embodiments, the compound has the structure of Formula Ia-1:

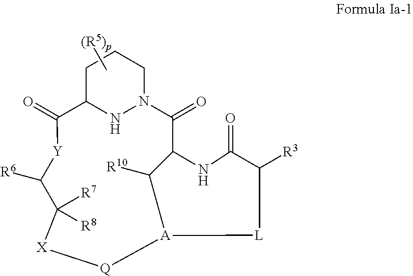

or pharmaceutically acceptable salt, an enantiomer, a stereoisomer, or a tautomer thereof, wherein
Q is an optionally substituted 7- to 12-membered bicyclic arylene, an optionally substituted 7- to 12-membered bicyclic heteroarylene, an optionally substituted 7- to -12 membered bicyclic heterocyclylene, wherein a first ring in Q is bonded to X, and a second ring in Q is bonded to A;
X is a bond; a straight chain C₁-C₃ alkylene optionally substituted with 1 to 3 substituents independently selected from fluoro, —CN, —C₁-C₃ alkyl, and —O—C₁-C₃ alkyl; —O—; —S(O)₀₋₂—; *—CH₂—O—; *—CH₂—S(O)₀₋₂—; *—O—CH₂—; or *—CH₂—S(O)₀₋₂—, wherein “*” represents a portion of X bound to —C(R⁷)(R⁸)—;
Y is —O—, —NH— or —N(C₁-C₃ alkyl)-;
A is optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
L is a linker;
R³ is optionally substituted C₁-C₆ alkyl, optionally substituted C₃-C₆ cycloalkyl, optionally substituted C₆ aryl, or optionally substituted 3- to 7-membered heterocyclyl;
R¹⁰ is hydrogen, halogen, optionally substituted C₁-C₃ alkyl, C₁-C₃ optionally substituted heteroalkyl, or C₁-C₃ optionally substituted hydroxyalkyl;
R⁷ is hydrogen, halogen, or optionally substituted C₁-C₃ alkyl;
R⁸ is hydrogen, halogen, —OH, —CN, —O-(optionally substituted C₁-C₃ alkyl), optionally substituted C₁-C₃ alkyl, optionally substituted C₂-C₆ alkenyl, optionally substituted C₂-C₆ alkynyl, optionally substituted C₆-C₁₀ aryl, optionally substituted 4- to 8-membered heteroaryl, optionally substituted C₃-C₆ cycloalkyl, or optionally substituted 3- to 7-membered heterocyclyl; or
R⁷ and R⁸ are taken together to form ═CH₂, an optionally substituted C₃-C₆ cycloalkyl, or a 3- to 7-membered saturated heterocyclyl; or
R⁸ is taken together with a ring atom in Q, the carbon atom to which R⁷ is bound and X to form a 4- to 9-membered saturated or unsaturated heterocyclyl that is fused to Q;
R⁶ is hydrogen or —CH₃;
each R⁵ is independently halogen, optionally substituted C₁-C₃ alkyl, or optionally substituted C₁-C₃ haloalkyl; and
p is 0, 1, 2, or 3.

In some embodiments, the compound has the structure of formula Ia-2:

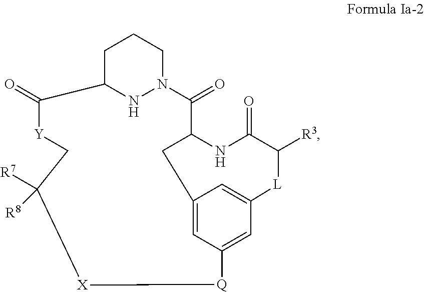

or a pharmaceutically acceptable salt, an enantiomer, a stereoisomer, or a tautomer thereof,
where:
X is a bond, —O—, —CH₂—, —CH(CH₃)—, *—CH₂—O—, or —CH₂—CH₂—, where “*” represents a portion of X bound to C(R⁴)(R⁵);
Y is —O— or —NH—;
L is a linker;
R³ is —C₁-C₄ alkyl, —(CH₂)₀₋₁—(C₃-C₆ cycloalkyl), or —C₄-C₆ cycloalkyl;
R⁷ is hydrogen, halo, or C₁-C₃ alkyl;
R⁸ is hydrogen, halo, —OH, C₁-C₃ alkyl, C₁-C₃ hydroxyalkyl, C₁-C₃ alkylene-O—C₁-C₃ alkyl, C₁-C₃ haloalkyl, —(CH₂)₀₋₁—C₃-C₆ cycloalkyl, C₁-C₃ cyanoalkyl, or —(CH₂)₀₋₁-aryl (benzyl), or
R⁷ and R⁸ are taken together to form ═CH₂, or a C₃-C₆ cycloalkyl, or
R⁸ is taken together with a ring atom of Q, the carbon atom to which it is bound and X to form a 5- to 7-membered saturated heterocyclyl;
Q is a bicyclic arylene, a bicyclic heteroarylene, or a bicyclic heterocyclylene, where:
a first ring in Q is bonded to X, and a second ring in Q is bonded Z; and
Q is optionally substituted with one or more independently selected substituents selected from ═O; —CN; halogen; —C₁-C₅ alkyl optionally substituted with one or more independently selected halo, CN, OH, —O—(C₁-C₃ alkyl), —C(O)—(C₁-C₃ alkyl), —O—(C₂-C₃ alkynyl), —(C₃-C₆ cycloalkyl), or a 4- to 7-membered saturated heterocyclyl; —O—(C₁-C₃ alkyl) optionally substituted with one or more independently selected halo; C₂-C₅ alkenyl optionally substituted with one or more independently selected —CN, or —OH; C₂-C₃ alkynyl; —S(O)₂—C₁-C₃ alkyl; —(CH₂)₀₋₁—C₃-C₆ cycloalkyl optionally substituted with one or more independently selected halo, ═O, —CN, C₁-C₃ alkyl optionally substituted with —CN or —O—C₁-C₃ alkyl, —C(O)-saturated heterocyclyl, —O-saturated heterocyclyl, O-cycloalkyl, or —O-aryl; —(CH₂)₀₋₁-heteroaryl optionally substituted with one or more independently selected halo, —CN, C₁-C₃ alkyl optionally substituted with —CN or —O—C₁-C₃ alkyl, —C(O)-saturated heterocyclyl, —O-saturated heterocyclyl, O-cycloalkyl, or —O-aryl; —(CH₂)₀₋₁-heterocyclyl optionally substituted with one or more independently selected halo, ═O, —CN, C₁-C₃ alkyl optionally substituted with —CN or —O—C₁-C₃ alkyl, —C(O)-saturated heterocyclyl, —O-saturated heterocyclyl, O-cycloalkyl, or —O-aryl; —(CH₂)₀₋₁-aryl optionally substituted with one or more independently selected halo, —CN, —C₁-C₃ alkyl optionally substituted with —CN or —O—C₁-C₃ alkyl, —C(O)-saturated heterocyclyl, —O-saturated heterocyclyl, O-cycloalkyl, or —O-aryl; —C(O)—NH—(C₁-C₃ alkyl); —C(O)—N(C₁-C₃ alkyl)₂; C₂-C₃ alkenylene ═N—O—(C₁-C₃ alkyl) optionally substituted with C₃-C₆ cycloalkyl; or
two substituents on the same or adjacent ring atoms of Q are taken together to form a 5- to 7-membered monocyclic ring or a 6- to 12-membered bicyclic ring optionally substituted with one or more independently selected halo, ═O, —CN, C₁-C₃ alkyl, or —O—C₁-C₃ alkyl; and fused to Q.

In some embodiments, the compound has the structure of formula Ia-3:

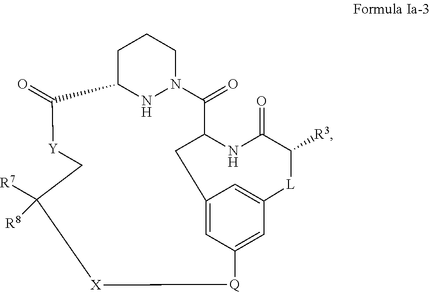

or a pharmaceutically acceptable salt, an enantiomer, a stereoisomer, or a tautomer thereof.

In some embodiments, the compound has the structure of formula (Ic):

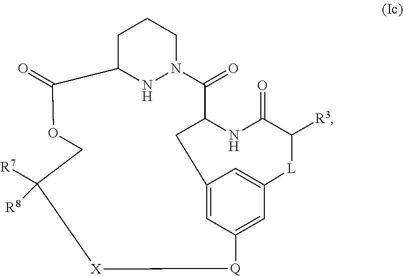

or a pharmaceutically acceptable salt, an enantiomer, a stereoisomer, or a tautomer thereof.

In some embodiments, Q is a 5,6 bicyclic heteroarylene, a 5,6 bicyclic heterocyclylene, a 6,6 bicyclic heteroarylene, or a 6,6 bicyclic heterocyclylene; and where Q is optionally substituted. In some embodiments, Q is a 5,6 bicyclic heteroarylene, wherein Q is optionally substituted. In some embodiments, Q is a 5,6 bicyclic heterocyclylene, wherein Q is optionally substituted. In some embodiments, Q is a 6,6 bicyclic heteroarylene, wherein Q is optionally substituted. In some embodiments, Q is a 6,6 bicyclic heterocyclylene, wherein Q is optionally substituted.

In some embodiments, Q is selected from the group consisting of:

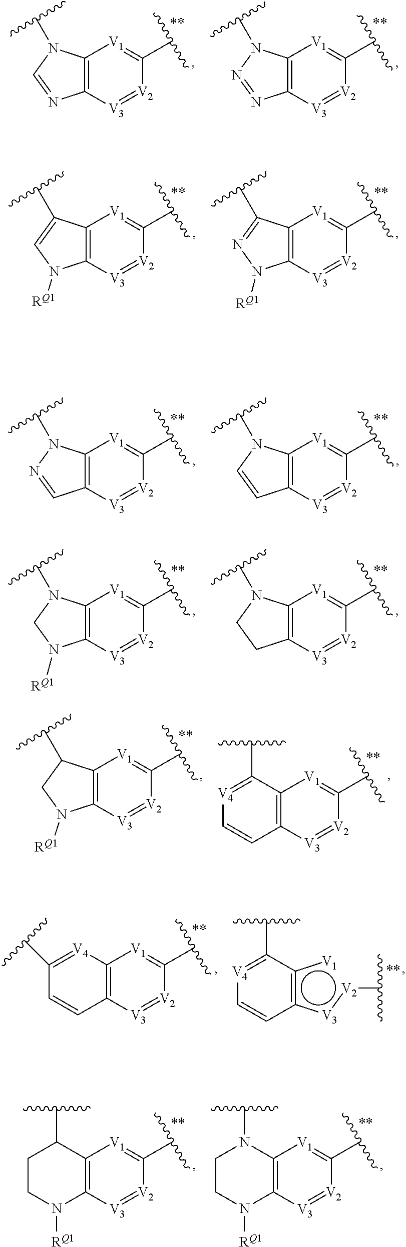

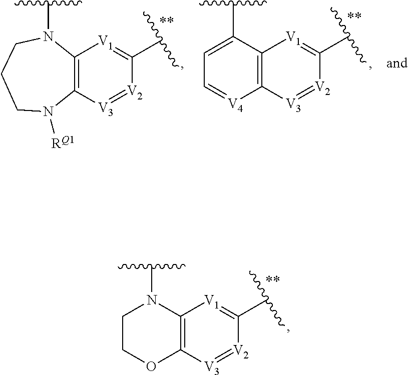

wherein:
each of V₁, V₂, V₃ and V₄ is independently C, CH, OF, or N;
R^{Q1} is —S(O)₂—R^{Q11}, —C(O)—R^{Q11}, —S(O)₂—N(R^{Q11})R^{Q12}, —C(O)—N(R^{Q11})R^{Q12}, C₁-C₁₀ alkyl, C₃-C₁₀ cycloalkyl, a 4- to 14-membered heterocyclyl, aryl, or heteroaryl, where the alkyl, cycloalkyl, heterocyclyl, aryl, or heteroaryl are optionally substituted; or
R^{Q1} is taken together with the nitrogen atom to which it is attached and an adjacent ring atom to form an optionally substituted 4- to 8-membered ring, which is optionally further fused to a 5- to 6-membered ring;
each of R^{Q11} and R^{Q12} is independently C₁-C₁₀ alkyl, C₃-C₁₀ cycloalkyl, a 4- to 14-membered heterocyclyl, aryl, or heteroaryl, where each of R^{Q11} and R^{Q12} is optionally substituted; or
R^{Q11} and R^{Q12} are taken together with the nitrogen atom to which they are both attached to form an optionally substituted 4- to 8-membered ring, where the ring formed by taking R^{Q11} and R^{Q12} together is optionally fused to another 5- to 6-membered ring.

In some embodiments, Q is optionally additionally substituted with 1 to 4 substituents independently selected from ═O; halo; —OH; —CN; —C₁-C₅ alkyl optionally substituted with one or more independently selected halo, CN, OH, —O—(C₁-C₃ alkyl), —C(O)—(C₁-C₃ alkyl), —O—C(O)—N(C₁-C₃ alkyl)₂, —O—(C₂-C₃ alkynyl), —(C₃-C₆ cycloalkyl), a 5- to 6-membered heteroaryl optionally substituted with one or more C₁-C₃ alkyl, or a 4- to 7-membered saturated heterocyclyl; —O—(C₁-C₃ alkyl) optionally substituted with one or more independently selected halo; —C₂-C₅ alkenyl optionally substituted with one or more independently selected —CN, or —OH; C₂-C₃ alkynyl optionally substituted with a heteroaryl; —S(O)₂—C₁-C₃ alkyl; —(CH₂)₀₋₁—C₃-C₆ cycloalkyl optionally substituted with one or more independently selected halo, ═O, —CN, C₁-C₃ alkyl optionally substituted with —CN or —O—C₁-C₃ alkyl, —C(O)-saturated heterocyclyl, —O-saturated heterocyclyl, O-cycloalkyl, or —O-aryl; —(CH₂)₀₋₁-heteroaryl optionally substituted with one or more independently selected halo, —CN, C₁-C₃ alkyl optionally substituted with —CN or —O—C₁-C₃ alkyl, —C(O)-saturated heterocyclyl, —O-saturated heterocyclyl, O-cycloalkyl, or —O-aryl; —(CH₂)₀₋₁-heterocyclyl optionally substituted with one or more independently selected halo, ═O, —CN, C₁-C₃ alkyl optionally substituted with —CN or —O—C₁-C₃ alkyl, —C(O)-saturated heterocyclyl, —O-saturated heterocyclyl, O-cycloalkyl, or —O-aryl; —(CH₂)₀₋₁-aryl optionally substituted with one or more independently selected halo, —CN, —C₁-C₃ alkyl optionally substituted with —CN, —C(O)—O—C₁-C₃ alkyl, —C₁-C₃ alkylene-O—C₁-C₃ alkyl, —O—C₁-C₃ alkyl, NO₂, —C(O)-saturated heterocyclyl, —CH₂-saturated heterocyclyl, —O-saturated heterocyclyl, O-cycloalkyl, or —O-aryl; —CH₂—O-heteroaryl, —C(O)—NH—(C₁-C₃ alkyl); —C(O)—N(C₁-C₃ alkyl)₂; C₂-C₃ alkenylene═N—O—(C₁-C₃ alkyl) optionally substituted with C₃-C₆ cycloalkyl; or
two substituents on Q are taken together to form a 5- to 7-membered monocyclic ring or a 6- to 12-membered bicyclic ring optionally substituted with one or more independently selected halo, ═O, —CN, C₁-C₃ alkyl, or —O—C₁-C₃ alkyl, and fused to Q; and
“**” represents a portion of Q that is bound to ring Z.

In some embodiments, Q is

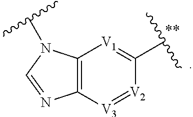

In some embodiments, Q is

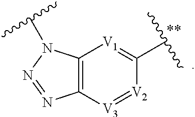

In some embodiments, Q is

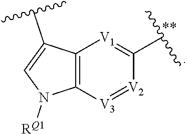

In some embodiments, Q is

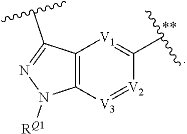

In some embodiments, Q is

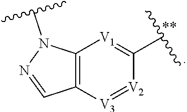

In some embodiments, Q is

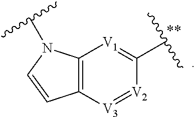

In some embodiments, Q is

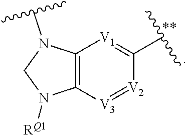

In some embodiments, Q is

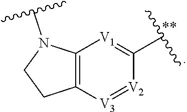

In some embodiments, Q is

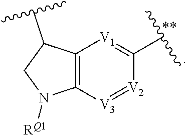

In some embodiments, Q is

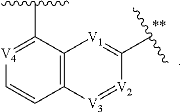

In some embodiments, Q is

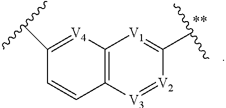

In some embodiments, Q is

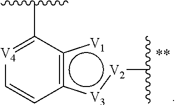

In some embodiments, Q is

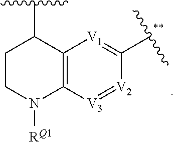

In some embodiments, Q is

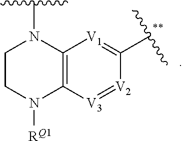

In some embodiments, Q is

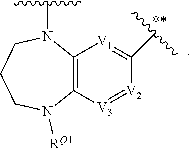

In some embodiments, Q is

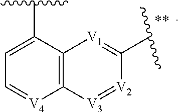

In some embodiments, Q is

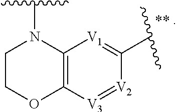

In some embodiments, Q is optionally additionally substituted with 1 to 4 substituents independently selected from chloro,

fluoro, —CN, —CH₃, —CF₃, —CHF₂, —CH₂CH₃, —CH₂—CN, —(CH₂)₂—CN, —OCH₃, —CH₂—O—CH₃, —(CH₂)₂—O—CH₃, —CH ₂—O—CH₂—CN, —CH(CN)—CH₃, —C(O)—N(CH₃)₂, —C(O)—NH—CH₃, —C(O)—CH₃, —S(O)₂CH₃, —C(CH₃)═NO—CH(CH₃)₂, —C(CH₃)═N—O—CH₃, —C═C—CH₃, —C═CH, —CH═CH—CN, —CH₂—O—CH₂—C═CH, —C(CH₃)(CN)CH₂CN, —CH₂—O—C(O)—N(CH₃)₂, 1-(cyclopentyl)-1-cyanoethan-1-yl, 1-(tetrahydrofuran-3-yl)-1-cyanoethan-1-yl, 1-(tetrahydropyran-4-yl)-1-cyanoethan-1-yl, 1,3-dimethoxy-2-cyanopropan-2-yl, 1,4-dimethylpyrazol-5-yl, 1-cyanocyclobutyl, 1-cyanocyclopropyl, 1-cyanocylopentyl, 1-methyl-1,2,3,6-tetrahydropyridin-4-yl, 1-methylpiperidin-4-yl, 1-methylpyrazol-3-yl, 1-methylpyrazol-5-yl, (1-methylpyrazol-4-yl)cyanomethyl, 1-oxoindolin-5-yl, 1-oxoisoindolin-4-yl, 1-oxoisoindolin-6-yl, 2-(2-methoxyethan-1-yl)phenyl, 3-(1,1-dioxothiomorpholin-1-ylmethyl)phenyl, 2-(tetrahydropyran-4-yloxy)phenyl, 2,2-difluoro-benzo[d][1,3]dioxol-4-yl, 2-chlorophenyl, 2-cyano-2-tetrahydrofuran-3-ylpropanyl, 2-cyano-3-chlorophenyl, 2-cyano-3-fluorophenyl, 2-cyano-3-methoxyphenyl, 2-cyano-4-fluorophenyl, 2-cyano-4-chlorophenyl, 2-cyano-4-methoxybutan-2-yl, 2-cyano-5-chlorophenyl, 2-cyano-5-fluorophenyl, 2-cyano-5-methoxyphenyl, 2-cyano-5-(methoxymethyl)phenyl, 2-cyano-6-chlorophenyl, 2-cyano-6-fluorophenyl, 2-cyano-6-bromophenyl, 2-cyano-6-(methoxymethyl)phenyl, 2-cyano-6-(tetrahydropyran-4-yloxy)phenyl, 2-cyanomethylphenyl, 2-cyanophenyl, 2-cyanopropan-2-yl, 2-cyclopentylphenyl, 2-difluoromethoxyphenyl, 2-fluorophenyl, 2-methoxy-6-cyanophenyl, 2-methoxyphenyl, 2-methoxycarbonylphenyl, 2-(methoxymethyl)phenyl, 2-nitrophenyl, 2-oxopyrrolidin-1-yl, 2-phenoxyphenyl, 3-(2-methoxyethan-1-yl)phenyl, 3-methoxycarbonylphenyl, 3,5-difluoro-4-(pyrrolidin-1-ylcarbonyl)phenyl, 3-cyano-2-methylpropan-2-yl, 3-cyanomethylphenyl, 3-cyanopentan-3-yl, 3-cyanophenyl, 3-hydroxy-2-methylbutan-2-yl, 3-hydroxy-3-methyl-but-1-yne-1-yl, 3-methoxy-2-methylbutan-2-yl, 3-methoxyphenyl, 3-methoxymethyl-5-methylisoxazol-4-yl, 3-oxo-2-methylbutan-2-yl, 3-(tetrahydropyran-4-yl)-2-cyanopropan-2-yl, 4-cyanophenyl, 4-cyanotetrahydropyran-4-yl, 4-methoxyphenyl, benzo[d][1,3]dioxol-4-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, cyclobutyl, cyclopropyl, cyclopropylcyanomethyl, morpholin-4-ylmethyl, N-methoxycyclopropanecarbimidoyl, phenyl, pyrazol-1-ylmethyl, pyridin-2-yl, pyridin-2-ylmethyl, pyridin-2-yloxymethyl, pyridin-3-yl, pyridin-3-yl-ethynyl, pyridin-3-ylmethyl, pyridin-4-ylmethyl, pyridin-4-yl-ethynyl, tetrahydrofuran-3-ylmethyl, tetrahydrofuran-3-ylcyanomethyl, tetrahydropyridin-4-yl, tetrahydropyran-4-ylmethyl, 2-(tetrahydropyran-4-yl)ethan-1-yl, tetrahydropyran-4-ylcyanomethyl, or tetrahydropyran-4-yl, or
two substituents attached to the same carbon atom are taken together to form ═O, 2,3-dihydrobenzofuran-3,3-diyl, 2,3-dihydrofuro[2,3-b]pyridin-3,3-diyl, tetrahydropyran-3,3-diyl, 6,7-dihydro-5H-cyclopenta[c]pyridin-6,6-diyl, or tetrahydropyran-4,4-diyl, or
two substituents attached to adjacent carbon atoms are taken together to form 4-cyanobenzene-1,2-diyl, 3-cyanobenzene-1,2-diyl, 5-methyl-5-cyanotetrahydropyran-3,4-diyl, 3-cyanocyclohexan-1,2-diyl, 3-methoxybenzene-1,2-diyl, benzene-1,2-diyl, 3-oxocyclohexyl-1,2-diyl, 3-cyanocyclopentan-1,2-diyl, or pyridin-3,4-diyl.

In some embodiments, Q is selected from the group consisting of:

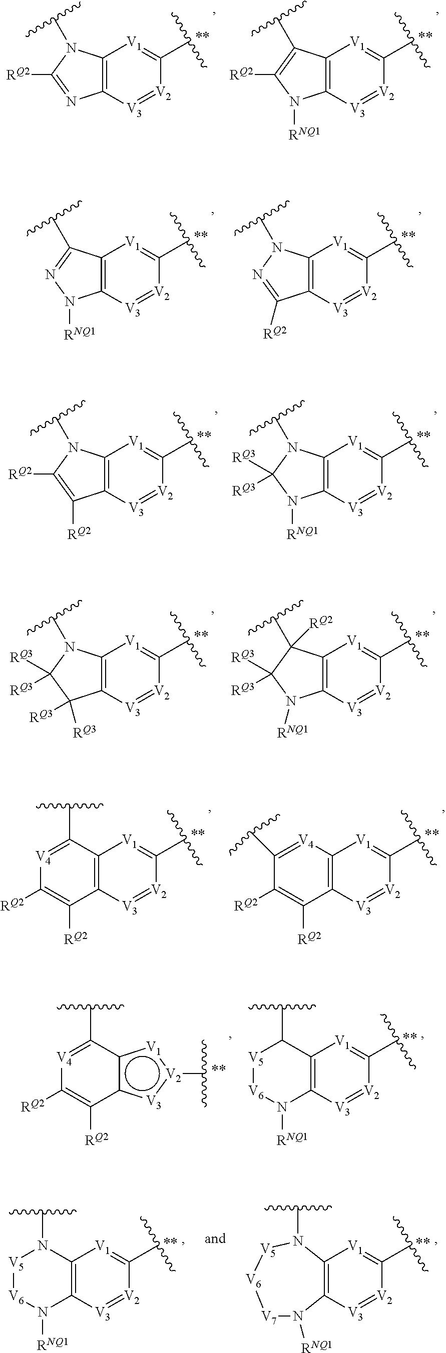

wherein:
each of V₁, V₂, V₃ and V₄ is independently CH, N, C(F), C(CH₃), C(OH), C(OCH₃), or C(CN);
each of V₅, V₆, and V₇ is independently, C(R^{17a})(R^{17b}), or C(═O), where each of R^{17a} and R^{17b} is independently selected from hydrogen, halo, —C₁-C₃ alkyl, —C₁-C₃ haloalkyl, —O—C₁-C₃ alkyl, —O—C₁-C₃ haloalkyl, and no more than two of V₅, V₆, and V₇ is C(═O);
R^{NQ1} is hydrogen, optionally
   substituted —S(O)₂—R^{Q11}, —C(O)—R^{Q11}, —S(O)₂—N(R^{Q11})R^{Q12}, —C(O)—N(R^{Q11})R^{Q12}, C₁-C₁₀ alkyl, C₃-C₁₀ cycloalkyl, a 4- to 14-membered heterocyclyl, aryl, or heteroaryl, where the alkyl, cycloalkyl, heterocyclyl, aryl, or heteroaryl are optionally substituted;
each R^{Q2} is independently hydrogen, CN, optionally
   substituted —S(O)₂—R^{Q11}, —C(O)—R^{Q11}, —S(O)₂—N(R^{Q11})R^{Q12}, —C(O)—N(R^{Q11})R^{Q12}, C₁-C₁₀ alkyl, C₃-C₁₀ cycloalkyl, a 4- to 14-membered heterocyclyl, aryl, or heteroaryl, where the alkyl, cycloalkyl, heterocyclyl, aryl, or heteroaryl are optionally substituted; or
R^{NQ1} and one R^{Q2} are taken together with the atoms to which they are bound to form an optionally substituted 4- to 8-membered ring, where the ring formed by taking R^{NQ1} and one R^{Q2} together is optionally further fused to a 5- to 6-membered ring;
each R^{Q3} is independently hydrogen, CN, optionally
   substituted —S(O)₂—R^{Q11}, —C(O)—R^{Q11}, —S(O)₂—N(R^{Q11})R^{Q12}, —C(O)—N(R^{Q11})R^{Q12}, C₁-C₁₀ alkyl, C₃-C₁₀ cycloalkyl, a 4-14 membered heterocyclyl, aryl, or heteroaryl, where the alkyl, cycloalkyl, heterocyclyl, aryl, or heteroaryl are optionally substituted, or
two R^{Q3} bound to the same atom are taken together to form ═CH, ═O, ═S, or ═NR^{V4}; or
two R^{Q3} bound to the same atom are taken together with the atom to which they are bound to form an optionally substituted 4- to 8-membered ring, where the ring formed by taking each R^{Q3} together is optionally further fused to a 5- to 6-membered ring; or
R^{NQ1} and one R^{Q3} are taken together with the atoms to which they are bound to form an optionally substituted 4- to 8-membered ring, where the ring formed by taking R^{NQ1} and R^{Q3} together is optionally further fused to a 5- to 6-membered ring;
each of R^{Q11} and R^{Q12} is independently C₁-C₁₀ alkyl, C₃-C₁₀ cycloalkyl, a 4- to 14-membered heterocyclyl, aryl, or heteroaryl, where each of R^{Q11} and R^{Q12} is optionally substituted; or
R^{Q11} and R^{Q12} are taken together with the atoms to which they are attached to form an optionally substituted 4- to 8-membered ring, where the ring formed by taking R^{Q11} and R^{Q12} together is optionally fused to another 5- to 6-membered ring; and
“**” represents a portion of Q that is bound to ring Z.

In some embodiments, Q is

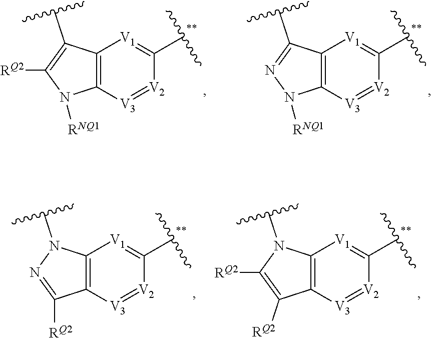

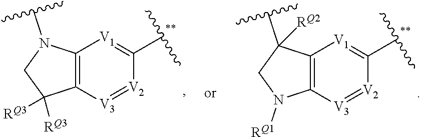

In some embodiments, Q is

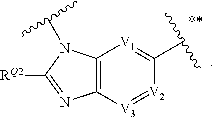

In some embodiments, Q is

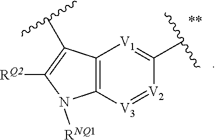

In some embodiments, Q is

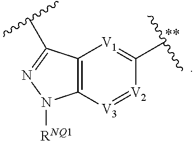

In some embodiments, Q is

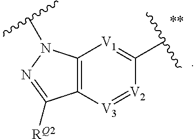

In some embodiments, Q is

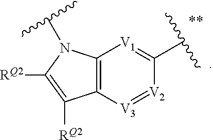

In some embodiments, Q is

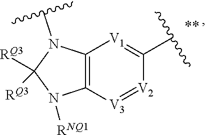

In some embodiments, Q is

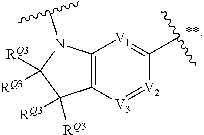

In some embodiments, Q is

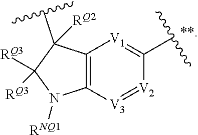

In some embodiments, Q is

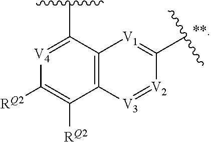

In some embodiments, Q is

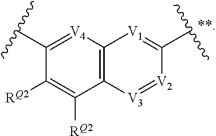

In some embodiments, Q is

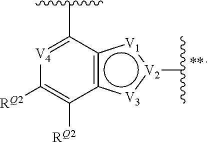

In some embodiments, Q is

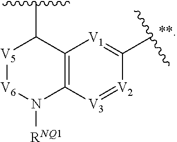

In some embodiments, Q is

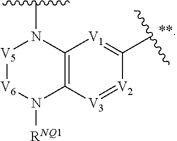

In some embodiments, Q is

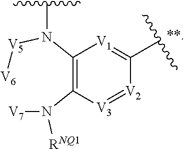

In some embodiments, Q is selected from the group consisting of:

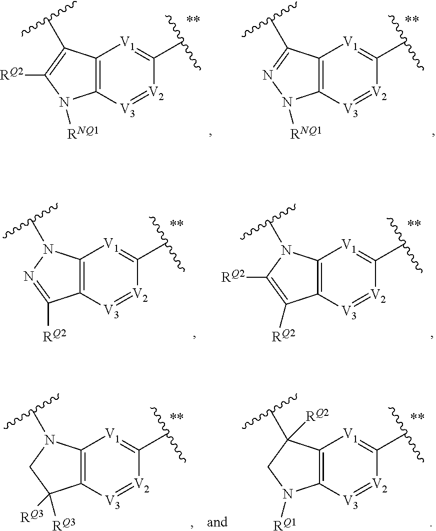

In some embodiments, Q is

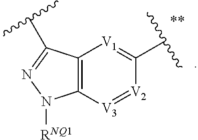

In some embodiments, Q is

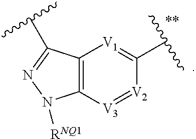

In some embodiments, Q is

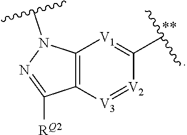

In some embodiments, Q is

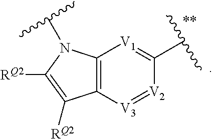

In some embodiments, Q is

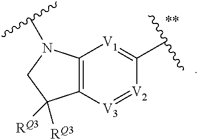

In some embodiments, Q is

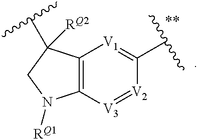

In some embodiments, the compound has the structure of formula (Id):

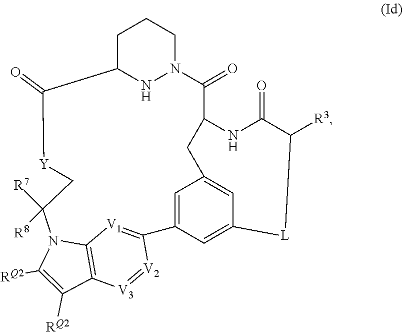

or a pharmaceutically acceptable salt, an enantiomer, a stereoisomer, or a tautomer thereof.

In some embodiments, the compound has the structure of formula (Ie):

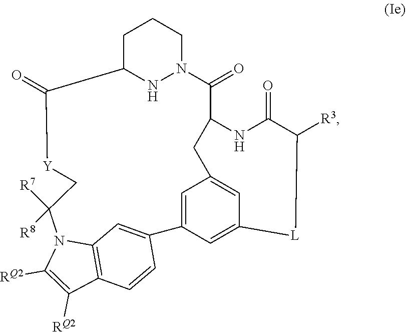

or a pharmaceutically acceptable salt, an enantiomer, a stereoisomer, or a tautomer thereof.

In some embodiments, the compound has the structure of formula (Ig):

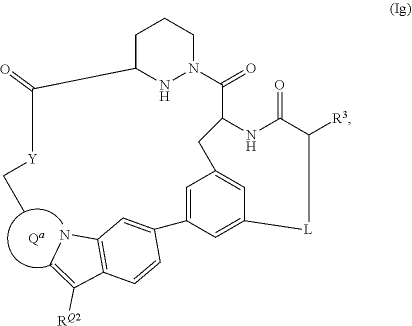

or a pharmaceutically acceptable salt, an enantiomer, a stereoisomer, or a tautomer thereof, where Q^{a} is a 4- to 9-membered saturated heterocyclyl.

In some embodiments, the compound has the structure of formula (Ij):

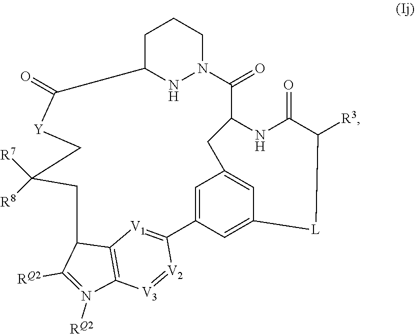

or a pharmaceutically acceptable salt, an enantiomer, a stereoisomer, or a tautomer thereof.

In some embodiments, the compound has the structure of formula (Ik):

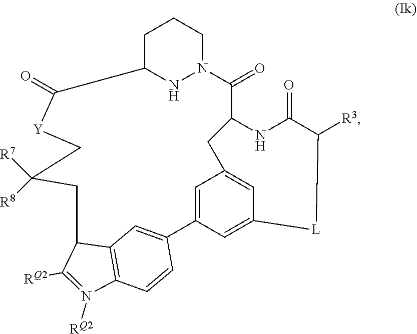

or a pharmaceutically acceptable salt, an enantiomer, a stereoisomer, or a tautomer thereof.

In some embodiments, the compound has the structure of formula (Ik′):

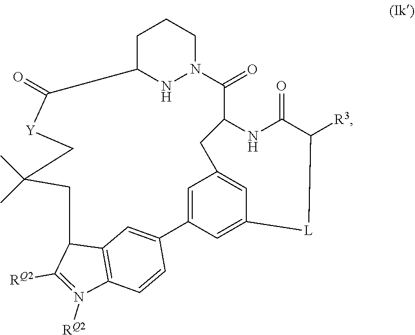

or a pharmaceutically acceptable salt, an enantiomer, a stereoisomer, or a tautomer thereof.

In some embodiments, Q is selected from the group consisting of:

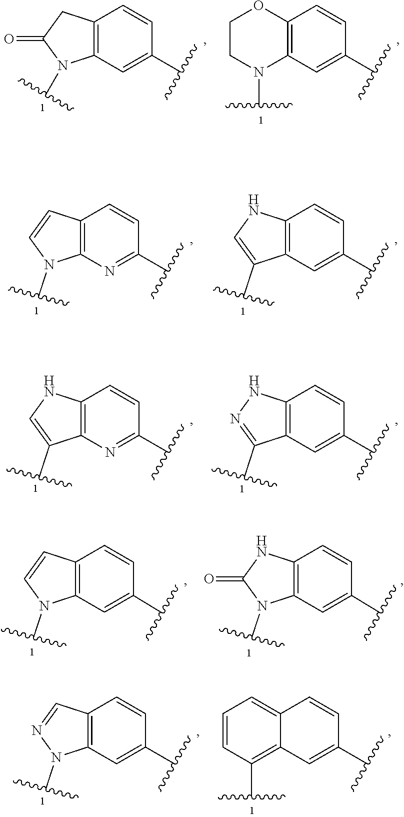

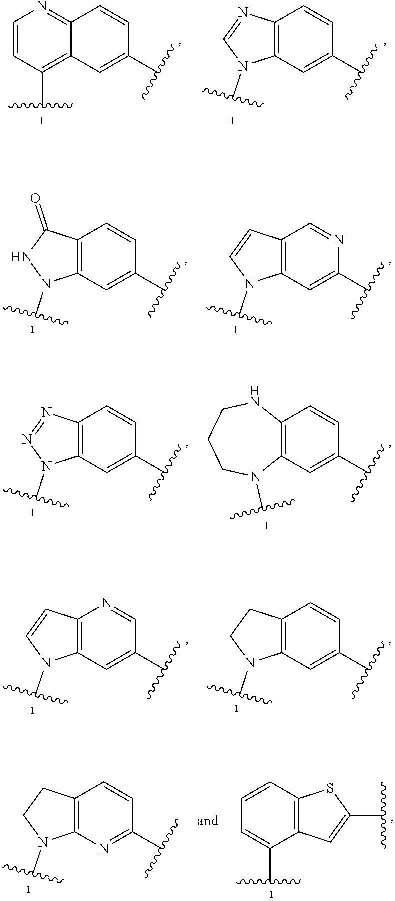

wherein:
“1” indicates a portion of Q bound to X; and Q is further optionally substituted. In some embodiments, Q is

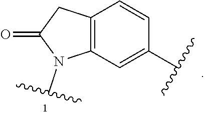 [SMILES: *c1ccc2c(c1)N(*)C(=O)C2]

In some embodiments, Q is

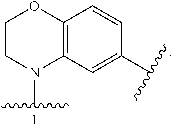 [SMILES: *c1ccc2c(c1)N(*)CCO2]

In some embodiments, Q is

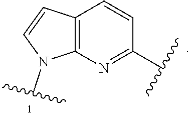 [SMILES: *c1ccc2ccn(*)c2n1]

In some embodiments, Q is

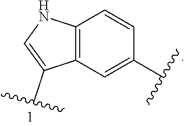 [SMILES: *c1ccc2[nH]cc(*)c2c1]

In some embodiments, Q is

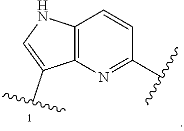 [SMILES: *c1ccc2[nH]cc(*)c2n1]

In some embodiments, Q is

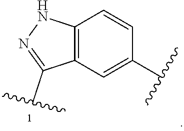 [SMILES: *c1ccc2[nH]nc(*)c2c1]

In some embodiments, Q is

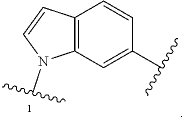 [SMILES: *c1ccc2ccn(*)c2c1]

In some embodiments, Q is

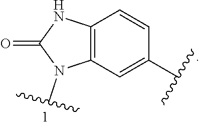 [SMILES: *c1ccc2[nH]c(=O)n(*)c2c1]

In some embodiments, Q is

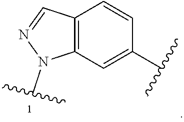 [SMILES: *c1ccc2cnn(*)c2c1]

In some embodiments, Q is

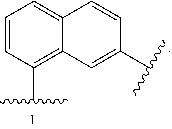 [SMILES: *c1ccc2cccc(*)c2c1]

In some embodiments, Q is

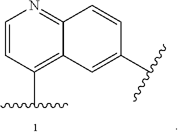 [SMILES: *c1ccc2nccc(*)c2c1]

In some embodiments, Q is

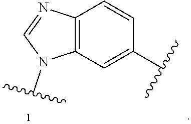 [SMILES: *c1ccc2ncn(*)c2c1]

In some embodiments, Q is. In some embodiments, Q is

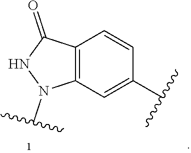 [SMILES: *c1ccc2c(=O)[nH]n(*)c2c1]

In some embodiments, Q is

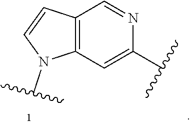 [SMILES: *c1cc2c(ccn2*)cn1]

In some embodiments, Q is

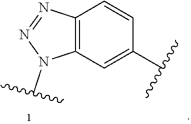 [SMILES: *c1ccc2nnn(*)c2c1]

In some embodiments, Q is

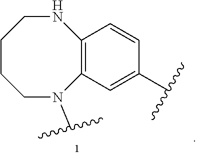 [SMILES: *c1ccc2c(c1)N(*)CCCCN2]

In some embodiments, Q is

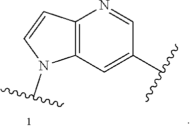 [SMILES: *c1cnc2ccn(*)c2c1]

In some embodiments, Q is

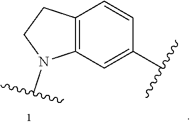 [SMILES: *c1ccc2c(c1)N(*)CC2]

In some embodiments, Q is

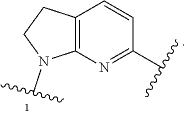 [SMILES: *c1ccc2c(n1)N(*)CC2]

In some embodiments, Q is

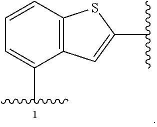 [SMILES: *c1cc2c(*)cccc2s1]

In some embodiments, Q is selected from the group consisting of:

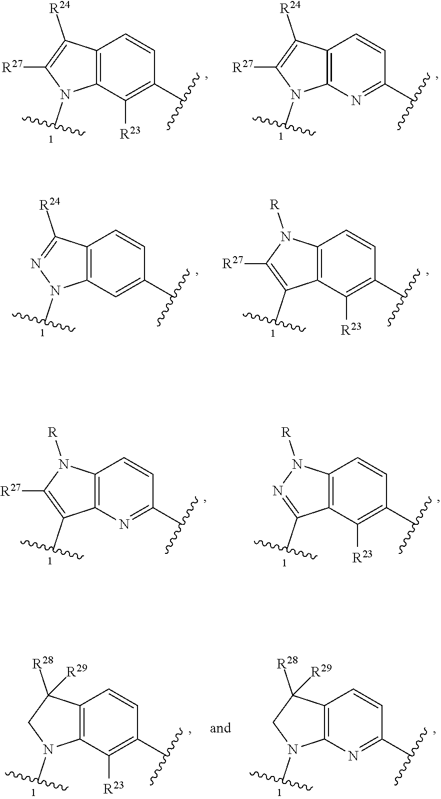

wherein:
R is —CH₂CH₃, —CH₂CH—OCH₃, —CH₂CHF₂, —CH₂—CN, CH₂(CH₃)₂—CN, —C(CH₃)₂—CH₂CN, —CH₂CH₂—CN, cyclohexyl, cyclobutyl, cyclopropyl, pyridin-4-yl, tetrahydropyran-4-yl, tetrahydropyran-4-ylmethyl, oxetan-3-ylmethyl, 2-cyano-5-methoxyphenyl, 2-cyano-5-methoxymethylphenyl, 2-cyano-6-(methoxymethyl)phenyl, 2-cyano-6-bromophenyl, 2-methoxyethan-1-yl, 2-cyanopropan-2-yl, 2-tetrahydropyran-4-ylethan-1-yl, 3-cyanopentan-3-yl, 2-cyano-4-methoxybutan-2-yl, or R is

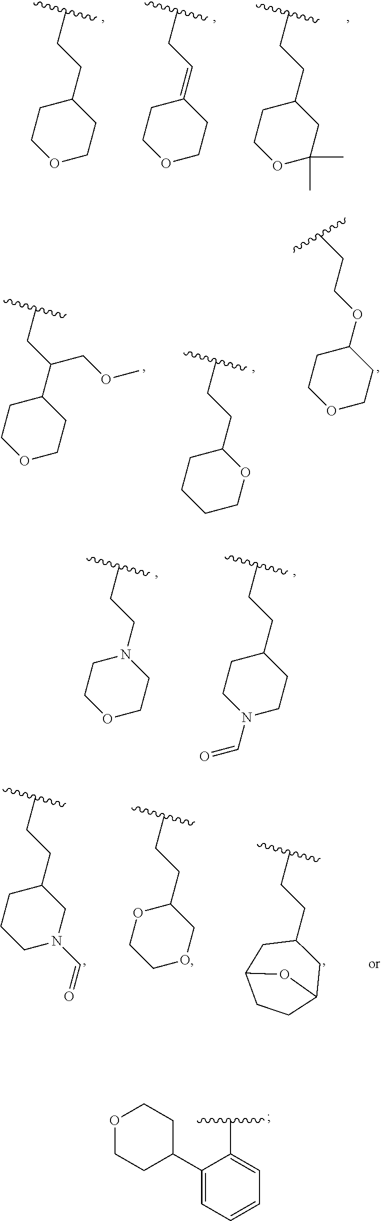

R²³ is hydrogen or halogen, such as fluoro;
R²⁴ is hydrogen, chloro, —CN, —CH₃, —CH₂CH₃, —CHF₂, —CF₃, —CH₂—CN, —CH(CN)—CH₃, —C(CH₃)₂—CN, —C(CH₂CH₃)₂—CN, —CH₂— CH₂—CN, —C(CH₃)═NO—CH(CH₃)₂, —C(CH₃)═N—O—CH₃, —C(O)—N(CH₃)₂, —C(O)—NH—CH₃, —OCH₃, —CH₂—O—CH₃, —CCH, —C═C—CH₃, —S(O)₂CH₃, 1-(cyclopentyl)-1-cyanoethan-1-yl, 1-(tetrahydropyran-4-yl)-1-cyanoethan-1-yl, 1-(tetrahydrofuran-3-yl)-1-cyanoethan-1-yl, 1,3-dimethoxy-2-cyanopropan-2-yl, 1,4-dimethylpyrazol-5-yl, 1-cyanocyclobutyl, 1-cyanocyclopropyl, 1-cyanocylopentyl, 1-methyl-1,2,3,6-tetrahydropyridin-4-yl, 1-methylpyrazol-3-yl, 1-methylpyrazol-4-ylcyanomethyl, 1-methylpiperidin-4-yl, 1-methylpyrazol-5-yl, 1-oxoindolin-5-yl, 1-oxoisoindolin-4-yl, 1-oxoisoindolin-6-yl, 2-(2-methoxyethan-1-yl)phenyl, 2-(methoxymethyl)phenyl, 2-(tetrahydropyran-4-yloxy)phenyl, 2,2-difluoro-benzo[d][1,3]dioxol-4-yl, 2,3-dicyanopropan-2-yl, 2-chlorophenyl, 2-cyano-3-(tetrahydropyran-4-yl)propan-2-yl, 2-cyano-3-chlorophenyl, 2-cyano-3-fluorophenyl, 2-cyano-3-methoxyphenyl, 2-cyano-4-fluorophenyl, 2-cyano-4-chlorophenyl, 2-cyano-5-chlorophenyl, 2-cyano-5-fluorophenyl, 2-cyano-5-methoxyphenyl, 2-cyano-6-chlorophenyl, 2-cyano-6-fluorophenyl, 2-cyano-6-(tetrahydropyran-4-yloxy)phenyl, 2-cyanomethylphenyl, 2-cyanophenyl, 2-cyanopropan-2-yl, 2-cyclopentylphenyl, 2-difluoromethoxyphenyl, 2-fluorophenyl, 2-methoxy-6-cyanophenyl, 2-methoxyphenyl, 2-methoxycarbonylphenyl, 2-nitrophenyl, 2-oxopyrrolidin-1-yl, 2-phenoxyphenyl, 3-(1,1-dioxothiomorpholin-4-ylmethyl)phenyl, 3-(2-methoxyethan-1-yl)phenyl, 3,5-difluoro-4-(pyrrolidin-1-ylcarbonyl)phenyl, 3-cyano-2-methylpropan-2-yl, 3-cyanomethylphenyl, 3-cyanopentan-3-yl , 3-cyanophenyl, 3-hydroxy-2-methylbutan-2-yl, 3-hydroxy-3-methyl-but-1-yne-1-yl, 3-methoxy-2-methylbutan-2-yl, 3-methoxymethyl-5-methylisoxazol-4-yl, 3-methoxyphenyl, 3-methoxycarbonylphenyl, 3-oxo-2-methylbutan-2-yl, 4-cyanophenyl, 4-cyanotetrahydropyran-4-yl, 4-methoxyphenyl, benzo[d][1,3]dioxol-4-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, cyclobutyl, cyclopropyl, cyclopropylcyanomethyl, N-methoxycyclopropanecarbimidoyl, phenyl, pyridin-2-ylmethyl, pyridin-3-yl, pyridin-3-ylmethyl, pyridin-4-ylmethyl, tetrahydrofuran-3-ylmethyl, tetrahydrofuran-3-ylcyanomethyl, tetrahydropyran-4-yl, or tetrahydropyran-4-ylcyanomethyl;
R²⁷ is hydrogen, —CH₃, —CHF₂, —CH₂CH₃, —CH₂—O—CH₃, —CH₂CN, —CN, —CH₂—O—CH₂—CN, —C(O)—N(CH₃)₂, —C(O)—NH—CH₃, —CH₂—O—CH₂—C═CH, 2-methoxyphenyl, 3-methoxyphenyl, 2,2-difluorobenzo[d][1,3]dioxol-4-yl, 2-cyanophenyl, 3-cyanophenyl, phenyl, 2-benzyl methyl ether, 2-(2-methoxyethyl)benzene, 2-(2-difluoromethoxyethyl)benzene, 2-(2-dimethylmethoxyethyl)benzene, pyridin-3-yl, pyridin-2-yl, pyridin-3-ylmethyl, or tetrahydropyridin-4-yl, or
R²⁴ and R²⁷ are taken together to form 4-cyanobenzene-1,2-diyl, 3-cyanobenzene-1,2-diyl, 5-methyl-5-cyanotetrahydropyran-3,4-diyl, 3-cyanocyclohexan-1,2-diyl, 3-methoxybenzene-1,2-diyl, benzene-1,2-diyl, 3-oxocyclohexyl-1,2-diyl, 3-cyanocyclopentan-1,2-diyl, or pyridin-3,4-diyl;
R²⁸ is hydrogen, —CH₃, or —CH₂—O—CH₃; and
R²⁹ is hydrogen, acetyl,
   CN, —CH₂—CN, —CH₂—CH₂—CN, —CH₂—O—CH₃, —CH═CH—CN, —CH₂—O—C(O)—N(CH₃)₂, morpholin-4-ylmethyl, pyrazol-1-ylmethyl, pyridin-3-yl, pyridin-3-ylethynyl, pyridin-2-yloxymethyl, or 2-cyanopropan-2-yl, or R²⁸ and R²⁹ are taken together to form 2,3-dihydrobenzofuran-3,3-diyl, 2,3-dihydrofuro[2,3-b]pyridin-3,3-diyl, tetrahydropyran-3,3-diyl, 6,7-dihydro-5H-cyclopenta[c]pyridin-6-yl, tetrahydropyran-4,4-diyl, or 4-methoxycyclohexane. In some embodiments, Q is

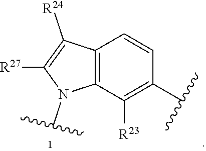

In some embodiments, Q is

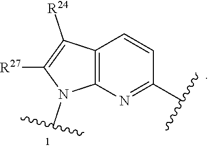

In some embodiments, Q is

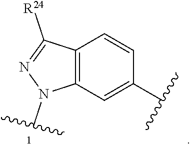

In some embodiments, Q is

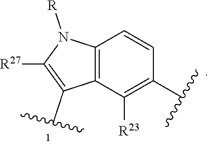

In some embodiments, Q is

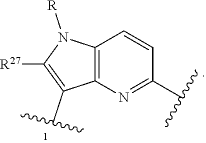

some embodiments, Q is

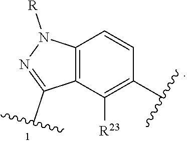

In some embodiments, Q is

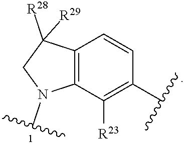

In some embodiments, Q is

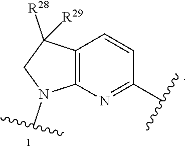

In some embodiments, R³ is —CH₃, —CH₂CH₃, —(CH₂)₂CH₃, —CH(CH₃)₂, —CH(CH₃)CH₂CH₃, cyclopropylmethyl, cyclobutylmethyl, cyclopentylmethyl, cyclohexylmethyl, cyclopropyl, cyclobutyl, cyclopentyl, cyclohexyl, phenyl, 4-methoxybenzyl, or tetrahydropyran-4-yl.

In an aspect, the invention features a compound having the structure of Formula IIa or Formula IIb:

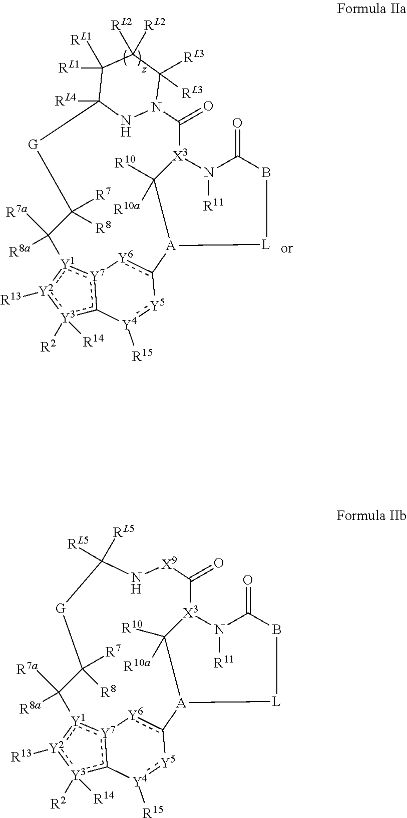

or pharmaceutically acceptable salt, an enantiomer, a stereoisomer, or a tautomer thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CR^{x}, CH, CH₂, or N;
Y⁶ is CR^{z}, C(O), CH, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{z} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R⁷ is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl;
R²¹ is hydrogen or optionally substituted C₁-C₃ alkyl;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In an aspect, the disclosure features a compound of structural Formula IIa-1:

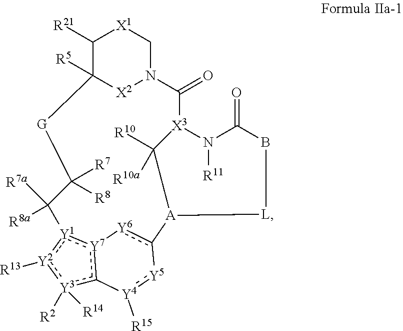

or pharmaceutically acceptable salt, an enantiomer, a stereoisomer, or a tautomer thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CR^{x}, CH, CH₂, or N;
Y⁶ is CR^{z}, C(O), CH, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{z} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl; and
R²¹ is hydrogen or optionally substituted C₁-C₃ alkyl.

In some embodiments, the disclosure features a compound of structural Formula IIa-2:

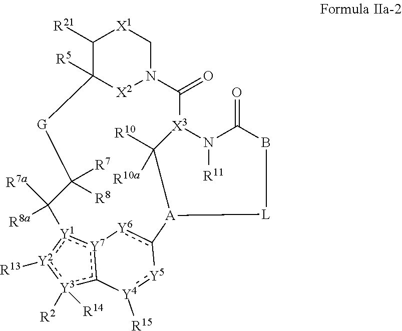

or pharmaceutically acceptable salt, an enantiomer, a stereoisomer, or a tautomer thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CH, CH₂, CF, CHF, CF₂ or N;
Y⁶ is C(O), CH, CH₂, CF, CHF, CF₂ or N;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, halogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl, or optionally substituted C₁-C₃ acyl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3- to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl; and
R²¹ is hydrogen or optionally substituted C₁-C₃ alkyl.

In some embodiments, provided herein is a compound having the structure of Formula IIa-3:

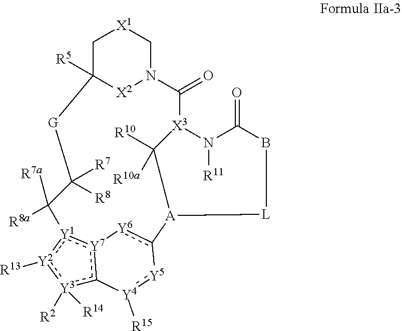

or pharmaceutically acceptable salt, an enantiomer, a stereoisomer, or a tautomer thereof, wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is —CH(R⁹)— or >C═CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, CF, CHF, CF₂ or N;
Y⁶ is C(O), CH, CH₂, CF, CHF, CF₂ or N;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, halogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl, or optionally substituted C₁-C₃ acyl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3- to 6-membered cycloalkyl or optionally substituted 3- to 7-membered heterocycloalkyl;
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl, or
R⁹ and L combine with the atoms to which they are attached to form an optionally substituted 3- to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound of structural Formula IIa-4:

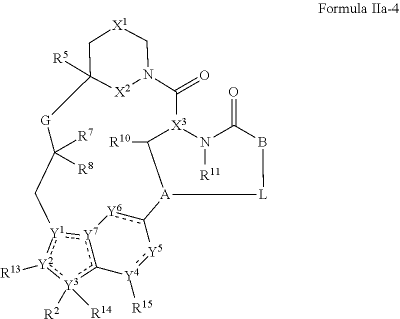

or pharmaceutically acceptable salt, an enantiomer, a stereoisomer, or a tautomer thereof, wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3- to 6-membered heterocycloalkylene, optionally substituted 6-membered arylene, or optionally substituted 5- to 6-membered heteroarylene;
B is —CH(R⁹)— where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH, CF or N;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6 to 10-membered aryl, or optionally substituted 5- to 10-membered heteroaryl;
R² is hydrogen, halogen, optionally substituted C₁-C₆ alkyl, optionally substituted C₂-C₆ alkenyl, optionally substituted 3 to 6-membered cycloalkyl, optionally substituted 3- to 7-membered heterocycloalkyl, optionally substituted 6-membered aryl, optionally substituted 5- or 6-membered heteroaryl, or optionally substituted C₁-C₃ acyl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3- to 6-membered cycloalkyl or optionally substituted 3- to 7-membered heterocycloalkyl;
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of compounds of the present invention, G is optionally substituted C₁-C₄ heteroalkylene.

In some embodiments, a compound having the structure of Formula IIa-5 is provided:

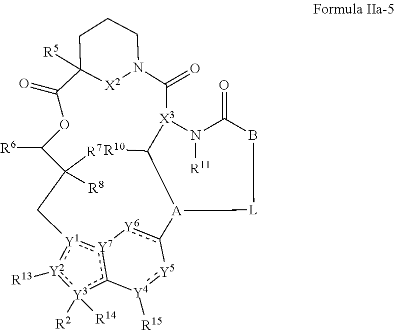

or pharmaceutically acceptable salt, an enantiomer, a stereoisomer, or a tautomer thereof, wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
B is —CH(R⁹)— where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is a cross-linking group comprising a vinyl ketone, a vinyl sulfone, an ynone, or an alkynyl sulfone;
X² is O or NH;
X³ is N or CH;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH, CF or N;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl;
R² is hydrogen, halogen, optionally substituted C₁-C₆ alkyl, optionally substituted C₂-C₆ alkenyl, optionally substituted 3- to 6-membered cycloalkyl, optionally substituted 3- to 7-membered heterocycloalkyl, optionally substituted 6-membered aryl, optionally substituted 5- or 6-membered heteroaryl, or optionally substituted C₁-C₃ acyl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or R^{7′} and R^{8′} combine with the carbon atom to which they are attached to form optionally substituted 3- to 6-membered cycloalkyl or optionally substituted 3- to 7-membered heterocycloalkyl;
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of compounds of the present invention, X² is NH. In some embodiments, X³ is CH. In some embodiments, R¹¹ is hydrogen. In some embodiments, R¹¹ is C₁-C₃ alkyl. In some embodiments, R¹¹ is methyl.

In some embodiments, a compound of the present invention has the structure of Formula IIa-6:

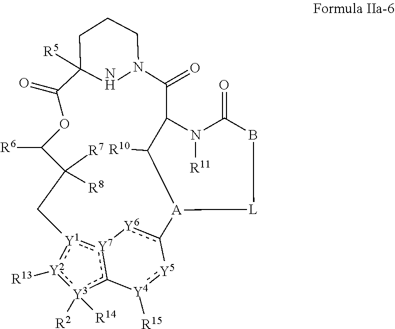

or pharmaceutically acceptable salt, an enantiomer, a stereoisomer, or a tautomer thereof, wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is a cross-linking group comprising a vinyl ketone, a vinyl sulfone, an ynone, or an alkynyl sulfone;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl;
R² is hydrogen, halogen, optionally substituted C₁-C₆ alkyl, optionally substituted C₂-C₆ alkenyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or R^{7′} and R^{8′} combine with the carbon atom to which they are attached to form optionally substituted 3- to 6-membered cycloalkyl or optionally substituted 3- to 7-membered heterocycloalkyl;
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; and
R¹⁰ is hydrogen, hydroxy, C₁-C₃ alkoxy, or C₁-C₃ alkyl.

In some embodiments of a compound of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH(Br)—. In some embodiments, X¹ is —CH(CH₃)—. In some embodiments, R⁵ is hydrogen. In some embodiments, R⁵ is C₁-C₄ alkyl optionally substituted with halogen. In some embodiments, R⁵ is methyl. In some embodiments, Y⁴ is C. In some embodiments, R⁴ is hydrogen. In some embodiments, Y⁵ is CH. In some embodiments, Y⁵ is CF. In some embodiments, Y⁶ is CH. In some embodiments, Y⁶ is CF. In some embodiments, Y¹ is C. In some embodiments, Y² is C. In some embodiments, Y³ is N. In some embodiments, R³ is absent. In some embodiments, Y⁷ is C. In some embodiments, Y⁴ is C and R¹⁵ is F.

In some embodiments, a compound of the present invention has the structure of Formula IIa-7:

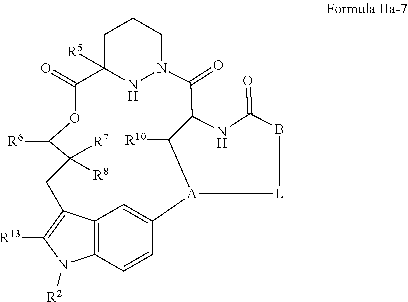

or pharmaceutically acceptable salt, an enantiomer, a stereoisomer, or a tautomer thereof, wherein A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is a cross-linking group comprising a vinyl ketone, a vinyl sulfone, an ynone, or an alkynyl sulfone;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or R^{7′} and R^{8′} combine with the carbon atom to which they are attached to form optionally substituted 3- to 6-membered cycloalkyl or optionally substituted 3- to 7-membered heterocycloalkyl;
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; and
R¹⁰ is hydrogen, hydroxy, C₁-C₃ alkoxy, or C₁-C₃ alkyl.

In some embodiments of a compound of the present invention, R⁶ is hydrogen. In some embodiments, R² is hydrogen, cyano, optionally substituted C₁-C₆ alkyl, optionally substituted 3- to 6-membered cycloalkyl, or optionally substituted 3- to 6-membered heterocycloalkyl. In some embodiments, R² is optionally substituted C₁-C₆ alkyl. In some embodiments, R² is fluoroalkyl. In some embodiments, R² is ethyl. In some embodiments, R₂ is —CH₂CF₃. In some embodiments, R₂ is C₂-C₆ alkynyl. In some embodiments, R₂ is —CHC≡CH. In some embodiments, R₂ is —CH₂C≡CCH₃. In some embodiments, R⁷ is optionally substituted C₁-C₃ alkyl. In some embodiments, R⁷ is C₁-C₃ alkyl. In some embodiments, R⁸ is optionally substituted C₁-C₃ alkyl. In some embodiments, R⁸ is C₁-C₃ alkyl.

In some embodiments, a compound of the present invention has the structure of Formula IIa-8:

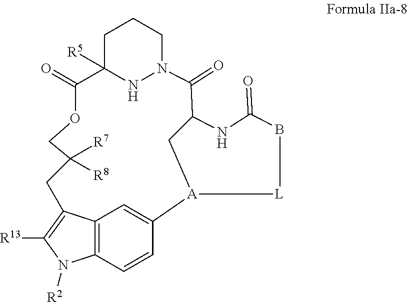

or pharmaceutically acceptable salt, an enantiomer, a stereoisomer, or a tautomer thereof, wherein A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is a cross-linking group comprising a vinyl ketone, a vinyl sulfone, an ynone, or an alkynyl sulfone;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl.

In some embodiments of a compound of the present invention, R¹³ is optionally substituted 6- to 10-membered aryl, optionally substituted 3- to 6-membered cycloalkenyl, or optionally substituted 5- to 10-membered heteroaryl. In some embodiments, R¹³ is optionally substituted 6-membered aryl, optionally substituted 6-membered cycloalkenyl, or optionally substituted 6-membered heteroaryl.

In some embodiments of a compound of the present invention, R¹³ is

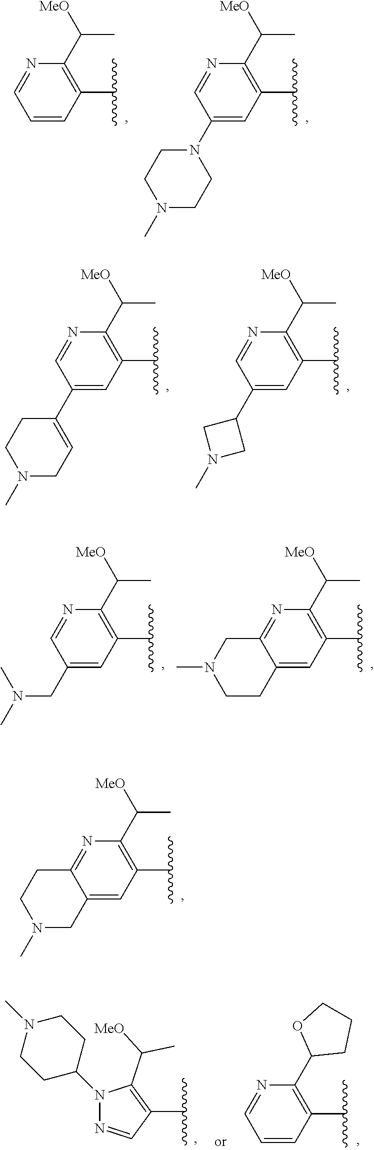

or a stereoisomer (e.g., atropisomer) thereof.

In some embodiments of a compound of the present invention, R¹³ is

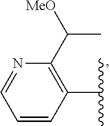 [SMILES: *c1cccnc1C(C)OC]

or a stereoisomer (e.g., atropisomer) thereof. In some embodiments of a compound of the present invention, R¹³ is

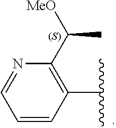

In some embodiments, R¹³ is

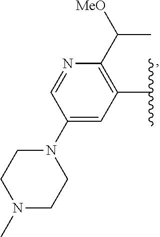 [SMILES: *c1cc(N2CCN(C)CC2)cnc1C(C)OC]

or a stereoisomer thereof. In some embodiments, R¹³ is

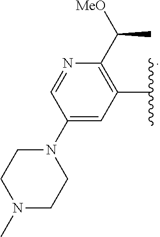 [SMILES: *c1cc(N2CCN(C)CC2)cnc1[C@H](C)OC]

In some embodiments, a compound of the present invention has the structure of Formula IIa-9:

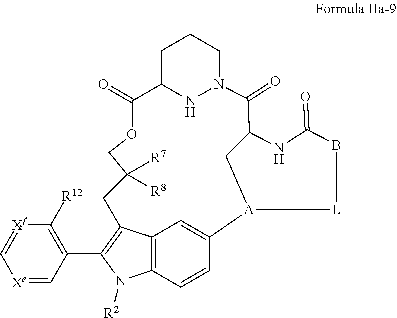

or pharmaceutically acceptable salt, an enantiomer, a stereoisomer, or a tautomer thereof, wherein A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is a cross-linking group comprising a vinyl ketone, a vinyl sulfone, an ynone, or an alkynyl sulfone;
R² is C₁-C₆ alkyl, C₁-C₆ fluoroalkyl, or 3- to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl
X^{e} and X^{f} are, independently, N, CH or CR¹⁷; and
R¹² is optionally substituted C₁-C₆ alkyl, optionally substituted C₁-C₆ heteroalkyl, or optionally substituted 3- to 6-membered heterocycloalkylene.

R¹⁷ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl.

In some embodiments of a compound of the present invention, X^{e} is N and X^{f} is CH. In some embodiments, X^{e} is CH and X^{f} is N.

In some embodiments of compounds of the present invention, R¹² is optionally substituted C₁-C₆ heteroalkyl. In some embodiments, R¹² is

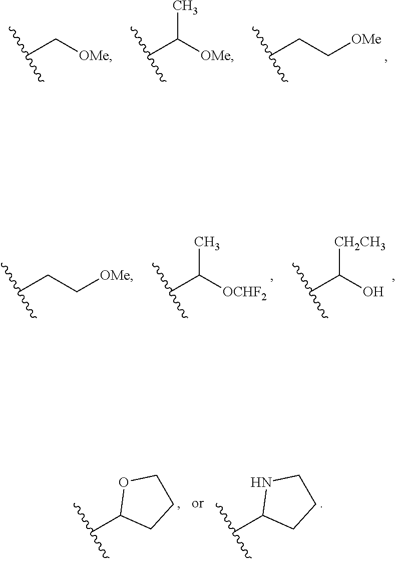

In some embodiments of a compound of the present invention, R¹² is optionally substituted C₁-C₆ heteroalkyl. In some embodiments, R¹² is

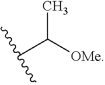 [SMILES: *C(C)OC]

In some embodiments, a compound of the present invention has the structure of Formula IIa-10:

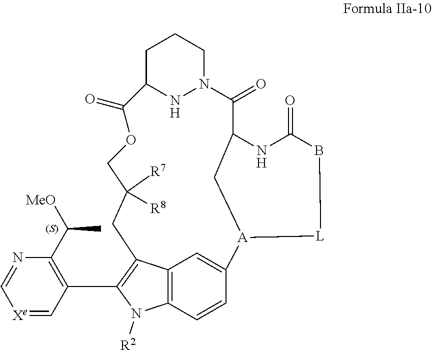

or pharmaceutically acceptable salt, an enantiomer, a stereoisomer, or a tautomer thereof, wherein A is optionally substituted 3- to 6-membered cycloalkylene, optionally substituted 3- to 6-membered heterocycloalkylene, optionally substituted 6-membered arylene, or optionally substituted 5- to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3- to 11-membered heterocycloalkyl, optionally substituted 3- to 8-membered cycloalkyl, or optionally substituted 3- to 8-membered heteroaryl;
R² is C₁-C₆ alkyl or 3- to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl;
X^{e} is CH, or CR¹⁷; and
R¹⁷ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl.

In some embodiments, a compound of the present invention has the structure of Formula IIa-11:

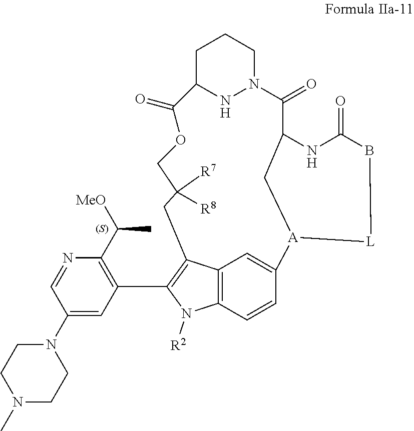

or pharmaceutically acceptable salt, an enantiomer, a stereoisomer, or a tautomer thereof, wherein A is optionally substituted 3- to 6-membered cycloalkylene, optionally substituted 3- to 6-membered heterocycloalkylene, optionally substituted 6-membered arylene, or optionally substituted 5- to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3- to 11-membered heterocycloalkyl, optionally substituted 3- to 8-membered cycloalkyl, or optionally substituted 3- to 8-membered heteroaryl;
R² is C₁-C₆ alkyl or 3- to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl.

In some embodiments, a compound of the present invention has the structure of Formula II-VI:

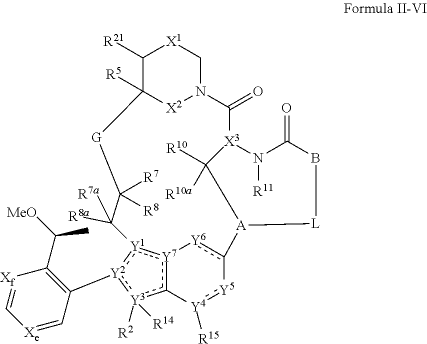

or pharmaceutically acceptable salt, an enantiomer, a stereoisomer, or a tautomer thereof, wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene (e.g., phenyl or phenol), or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂; each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, CF, CHF, CF₂ or N;
Y⁶ is C(O), CH, CH₂, CF, CHF, CF₂ or N;
R² is absent, hydrogen, halogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl, or optionally substituted C₁-C₃ acyl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3- to 6-membered cycloalkyl or optionally substituted 3- to 7-membered heterocycloalkyl;
R⁹ is H, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R²¹ is hydrogen or C₁-C₃ alkyl (e.g., methyl); and
X^{e} and X^{f} are, independently, N or CH.

In some embodiments, a compound of the present invention has the structure of Formula II-VIa:

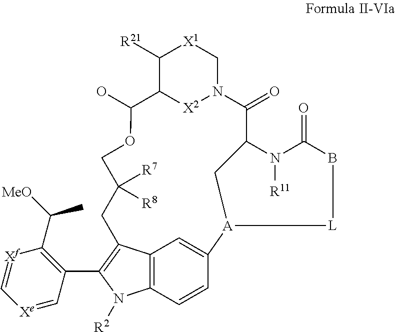

or pharmaceutically acceptable salt, an enantiomer, a stereoisomer, or a tautomer thereof, wherein A optionally substituted 3- to 6-membered cycloalkylene, optionally substituted 3- to 6-membered heterocycloalkylene, optionally substituted 6-membered arylene (e.g., phenyl or phenol), or optionally substituted 5- to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)_{q};
X² is O or NH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3- to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of a compound of the present invention, X^{e} is N and X^{f} is CH. In some embodiments, X^{e} is CH and X^{f} is N.

In some embodiments, a compound of the present invention has the structure of Formula II-VIb:

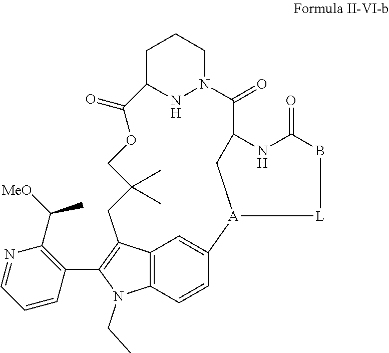

or pharmaceutically acceptable salt, an enantiomer, a stereoisomer, or a tautomer thereof, wherein A optionally substituted 3- to 6-membered cycloalkylene, optionally substituted 3- to 6-membered heterocycloalkylene, optionally substituted 6-membered arylene (e.g., phenyl or phenol), or optionally substituted 5- to 6-membered heteroarylene;
B is —CH(R⁹)— where the carbon is bound to the carbonyl carbon of —NHC(O)—, optionally substituted 3- to 6-membered cycloalkylene, optionally substituted 3- to 6-membered heterocycloalkylene, optionally substituted 6-membered arylene, or 5- to 6-membered heteroarylene;
R⁹ is optionally substituted C₁-C₆ alkyl, optionally substituted C₁-C₆ heteroalkyl, optionally substituted 3- to 6-membered cycloalkyl, or optionally substituted 3- to 7-membered heterocycloalkyl;
L is absent or a linker; and
W is a cross-linking group comprising a vinyl ketone, a vinyl sulfone, an ynone, or an alkynyl sulfone. In some embodiments of a compound of the present invention, A is optionally substituted 6-membered arylene.

In some embodiments, a compound of the present invention has the structure of Formula II-VIc:

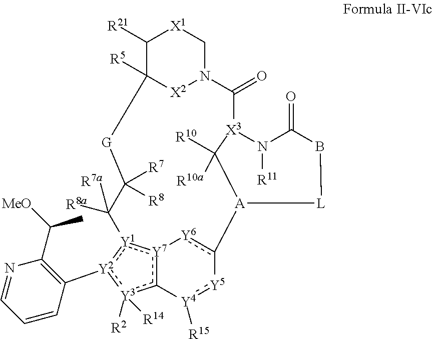

or pharmaceutically acceptable salt, an enantiomer, a stereoisomer, or a tautomer thereof, wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3- to 6-membered cycloalkylene, optionally substituted 3- to 6-membered heterocycloalkylene, optionally substituted 6-membered arylene (e.g., phenyl or phenol), or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is absent or a linker;
W is a cross-linking group comprising a vinyl ketone, a vinyl sulfone, an ynone, a haloacetal, or an alkynyl sulfone;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
   each R′ is, independently, H or optionally substituted C₁-C₄ alkyl;

### Y¹ is C, CH, or N;

Y², Y³, Y⁴, and Y⁷ are, independently, C or N;
Y⁵ is CH, CH₂, CF, CHF, CF₂ or N;
Y⁶ is C(O), CH, CH₂, CF, CHF, CF₂ or N;
R² is absent, hydrogen, halogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl, or optionally substituted C₁-C₃ acyl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3- to 6-membered cycloalkyl or optionally substituted 3- to 7-membered heterocycloalkyl;
R⁹ is H, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments, Z is —C(O)—.

In some embodiments, A is optionally substituted C₂-C₄ alkylene. In some embodiments, A is optionally substituted C₃ alkylene. In some embodiments, A is:

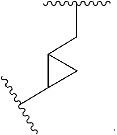 [SMILES: *CC1CC1*]

In some embodiments, A is optionally substituted C₂-C₄ alkenylene. In some embodiments, A is optionally substituted C₃ alkenylene. In some embodiments, A is optionally substituted C₁-C₄ heteroalkylene. In some embodiments, A is optionally substituted C₂ heteroalkylene. In some embodiments, A is

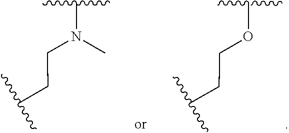

In some embodiments, A has the structure:

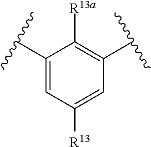

wherein R¹³ is hydrogen, halo, hydroxy, amino, optionally substituted C₁-C₆ alkyl, or optionally substituted C₁-C₆ heteroalkyl; and R^{13a} is hydrogen or halogen. In some embodiments, R¹³ is hydrogen. In some embodiments, R¹³ and R^{13a} are each hydrogen. In some embodiments, R¹³ is hydroxy, methyl, fluoro, or difluoromethyl.

In some embodiments, A is an optionally substituted 5- to 10-membered heteroarylene. In some embodiments, A is:

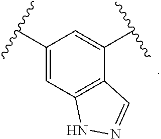 [SMILES: *c1cc(*)c2cn[nH]c2c1]

In some embodiments, A is optionally substituted 5- to 6-membered heteroarylene. In some embodiments, A is:

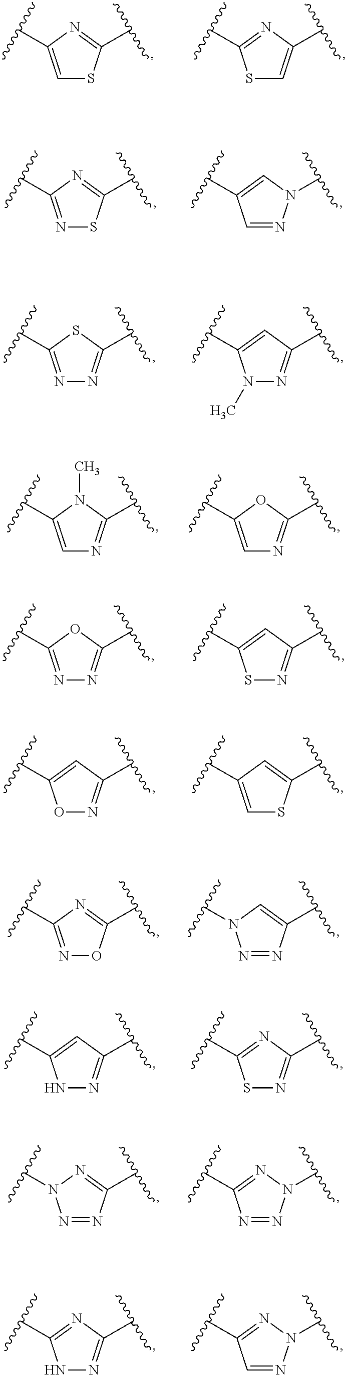

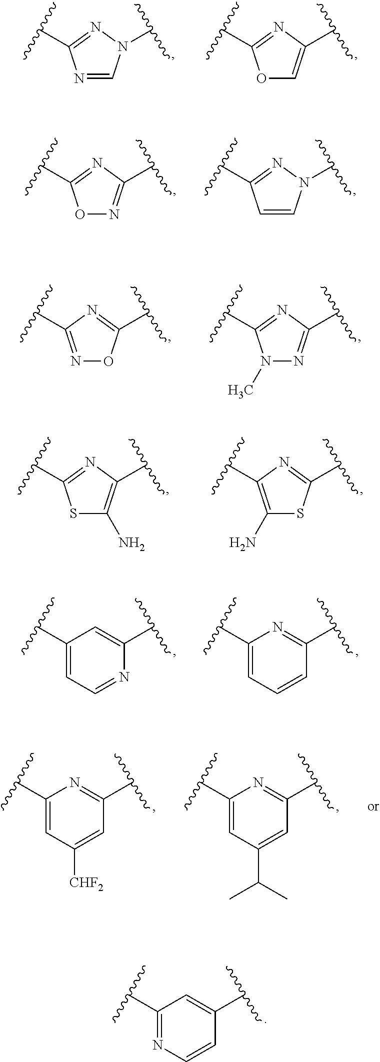

In some embodiments, A is optionally substituted C₁-C₄ heteroalkylene. In some embodiments, A is:

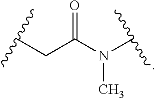 [SMILES: *CC(=O)N(*)C]

In some embodiments, A is optionally substituted 3- to 6-membered heterocycloalkylene. In some embodiments, A is:

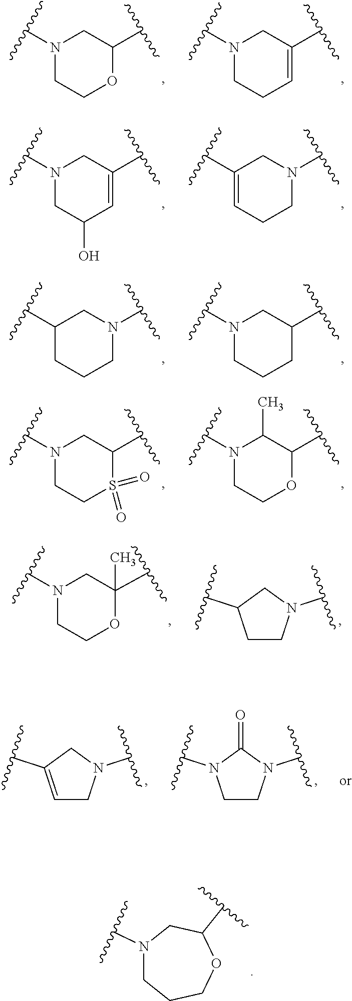

In some embodiments, A is

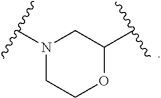 [SMILES: *C1CN(*)CCO1]

In some embodiments, R⁹ is H, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl. In some embodiments, R⁹ is:

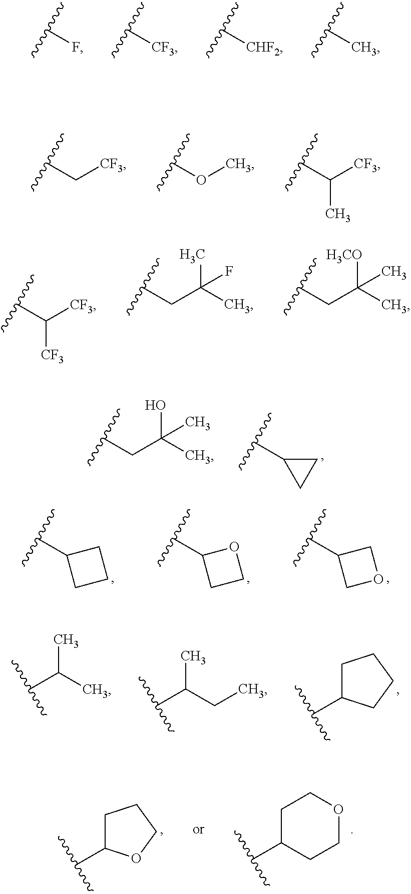

In some embodiments, R⁹ is:

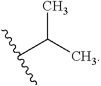 [SMILES: *C(C)C]

In some embodiments, R⁹ is H, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl.

In some embodiments of a compound of the present invention, B is optionally substituted 6-membered arylene. In some embodiments, B is 6-membered arylene. In some embodiments, B is:

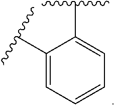 [SMILES: *c1ccccc1*]

In some embodiments, R¹³

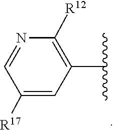

In some embodiments, R¹³ is

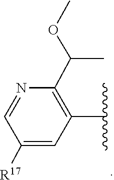

In some embodiments, R¹³ is

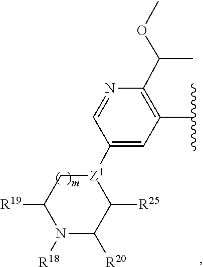

wherein Z¹ is N or CH;
m is 1 or 2;
R¹⁸, R¹⁹, R²⁰, and R²⁵ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R¹⁸ and R²⁰ combine with the atoms to which they are attached to form an optionally substituted 3- to 8-membered cycloalkyl or an optionally substituted 3- to 8-membered heterocycloalkyl; or
R²⁰ and R²⁵ combine with the atoms to which they are attached to form an optionally substituted 3- to 8-membered heterocycloalkyl; or
R¹⁹ and R²⁰ combine with the atoms to which they are attached to form an optionally substituted 4- to 8-membered heterocycloalkyl.

In some embodiments, R¹³

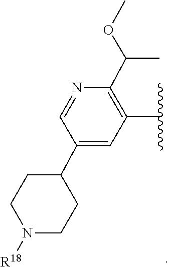

In some embodiments, R¹³ is

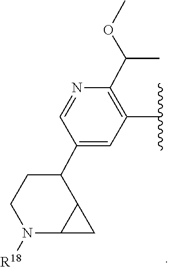

In some embodiments, R¹⁸ is methyl.

In some embodiments, R¹³ is

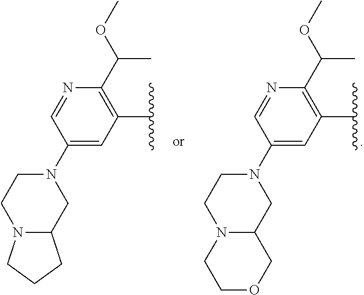 [SMILES: *c1cc(N2CCN3CCCC3C2)cnc1C(C)OC ; *c1cc(N2CCN3CCOCC3C2)cnc1C(C)OC]

In some embodiments of a compound of the present invention, R⁷ is methyl.

In some embodiments of a compound of the present invention, R⁸ is methyl.

In some embodiments, R²¹ is hydrogen.

In some embodiments of a compound of the present invention, B is —CHR⁹—. In some embodiments, R⁹ is optionally substituted C₁-C₆ alkyl or optionally substituted 3- to 6-membered cycloalkyl. In some embodiments, B is optionally substituted 6-membered arylene. In some embodiments, B is absent.

In some embodiments, L has the structure of Formula L₀:

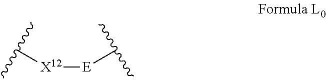

wherein X¹² is O, S, SO₂, NH, CH₂, C₁-C₄ alkylene, optionally substituted C₁-C₄ heteroalkylene, optionally substituted C₂-C₄ alkenylene, or optionally substituted C₂-C₄ alkynylene, and is attached to ring A; and
E is a bond, optionally substituted C₁-C₆ alkylene, optionally substituted C₁-C₆ heteroalkylene, optionally substituted C₂-C₆ alkenylene, optionally substituted 3- to 8-membered cycloalkylene, optionally substituted 3- to 8-membered heterocycloalkylene, optionally substituted 5- to 12-membered arylene, or an optionally substituted 5- to 12-membered heteroarylene.

In some embodiments of a compound of the present invention, the linker is the structure of Formula II-II:

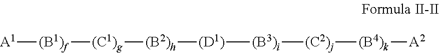

where A¹ is a bond between the linker and B; A² is a bond between A and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂-C₄ alkenyl, optionally substituted C₂-C₄ alkynyl, optionally substituted 3- to 14-membered heterocycloalkyl, optionally substituted 6- to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3- to 14-membered heterocycloalkylene, optionally substituted 5- to 10-membered heteroarylene, optionally substituted 3- to 8-membered cycloalkylene, optionally substituted 6- to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments, the linker is acyclic. In some embodiments, linker has the structure of Formula II-IIa:

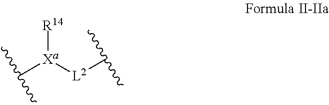

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

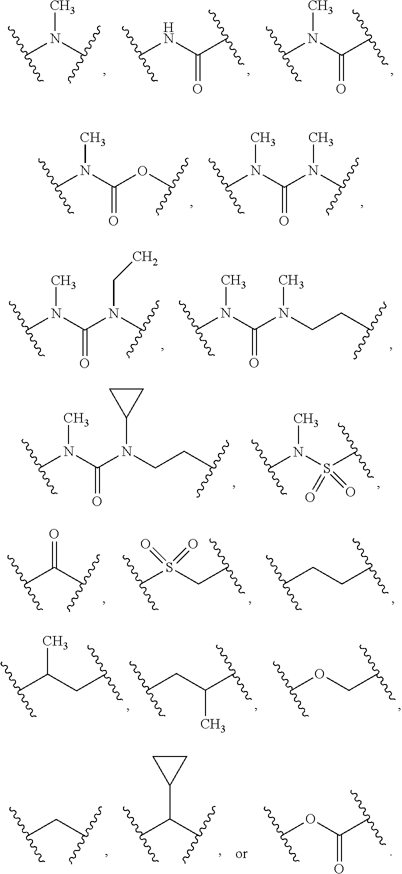

In some embodiments, the linker has the structure:

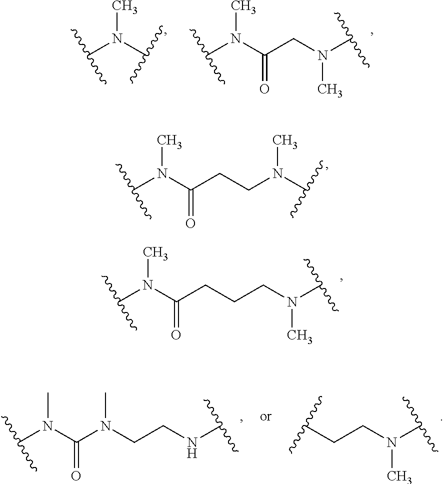

In some embodiments, L is

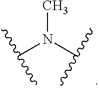 [SMILES: *N(*)C]

In some embodiments, L is

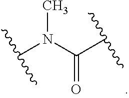 [SMILES: *C(=O)N(*)C]

In some embodiments, the linker is or comprises a cyclic moiety. In some embodiments, the linker has the structure of Formula II-IIb:

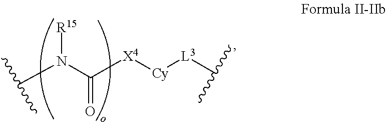

wherein o is 0 or 1;
R¹⁵ is hydrogen or optionally substituted C₁-C₆ alkyl, optionally substituted 3- to 8-membered cycloalkylene, or optionally substituted 3- to 8-membered heterocycloalkylene;
X⁴ is absent, optionally substituted C₁-C₄ alkylene, O, NCH₃, or optionally substituted C₁-C₄ heteroalkylene;
Cy is optionally substituted 3- to 8-membered cycloalkylene, optionally substituted 3- to 8-membered heterocycloalkylene, optionally substituted 6- to 10-membered arylene, or optionally substituted 5- to 10-membered heteroarylene; and
L³ is absent, —SO₂—, optionally substituted C₁-C₄ alkylene or optionally substituted C₁-C₄ heteroalkylene.

In some embodiments, the linker has the structure of Formula II-IIb-1:

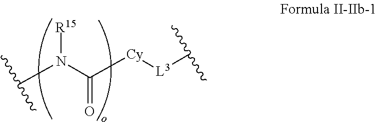

wherein o is 0 or 1;
R¹⁵ is hydrogen or optionally substituted C₁-C₆ alkyl, optionally substituted 3- to 8-membered cycloalkylene, or optionally substituted 3- to 8-membered heterocycloalkylene;
Cy is optionally substituted 3- to 8-membered cycloalkylene, optionally substituted 3- to 8-membered heterocycloalkylene, optionally substituted 6- to 10-membered arylene, or optionally substituted 5- to 10-membered heteroarylene; and
L³ is absent, —SO₂—, optionally substituted C₁-C₄ alkylene or optionally substituted C₁-C₄ heteroalkylene.

In some embodiments, the linker has the structure:

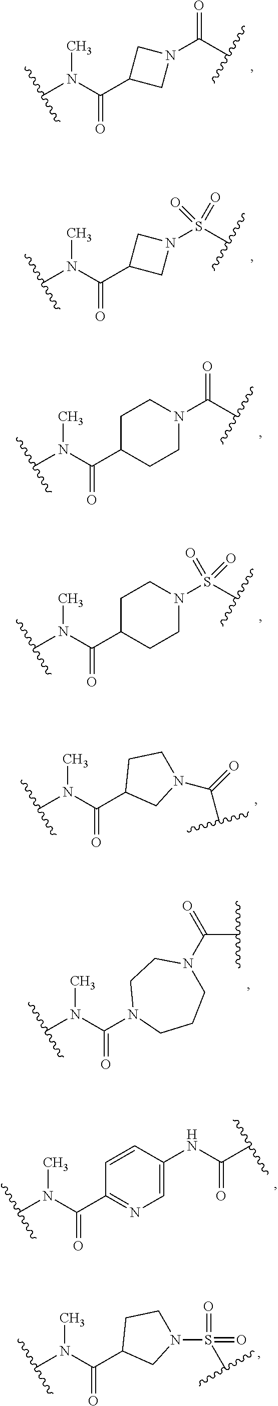

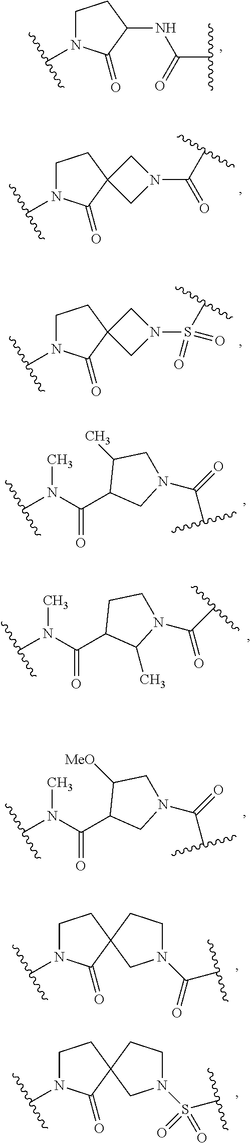

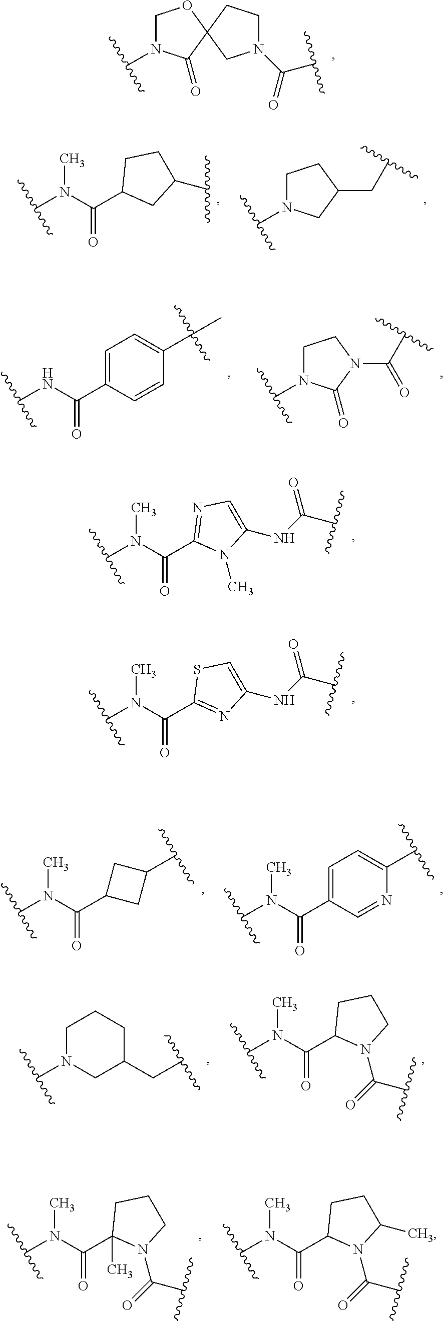

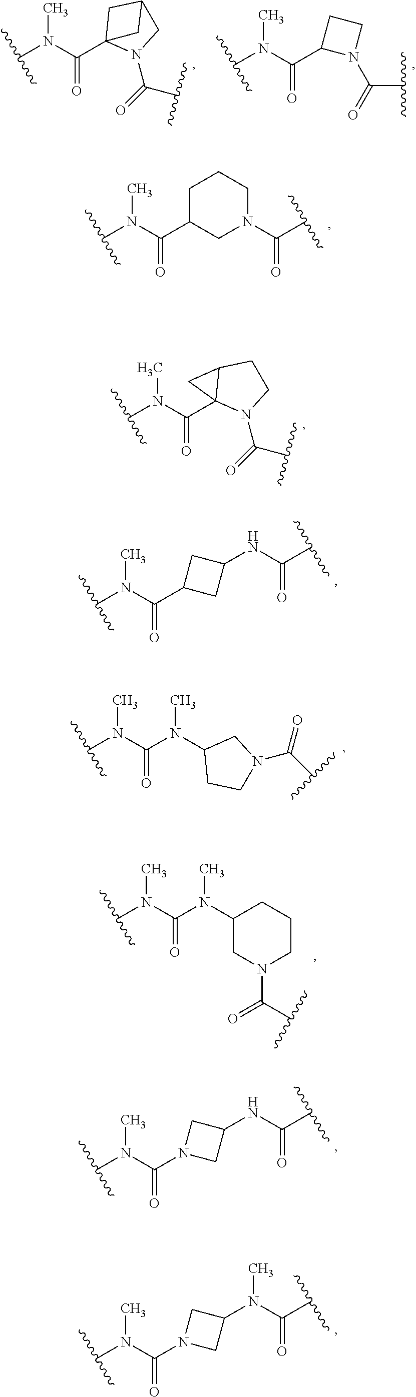

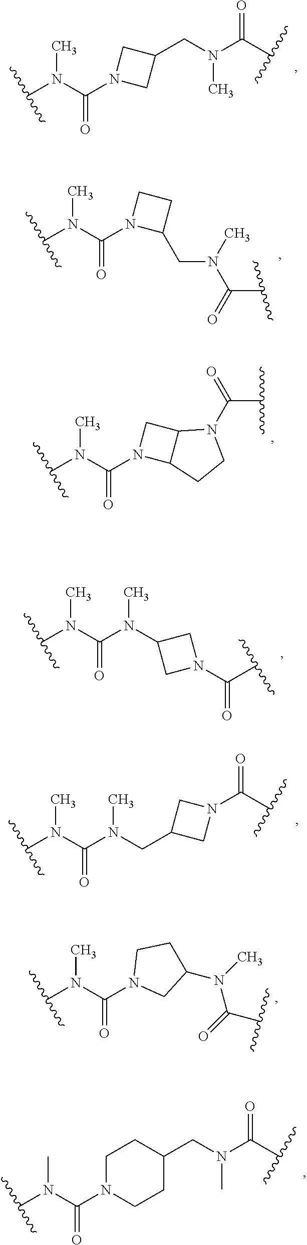

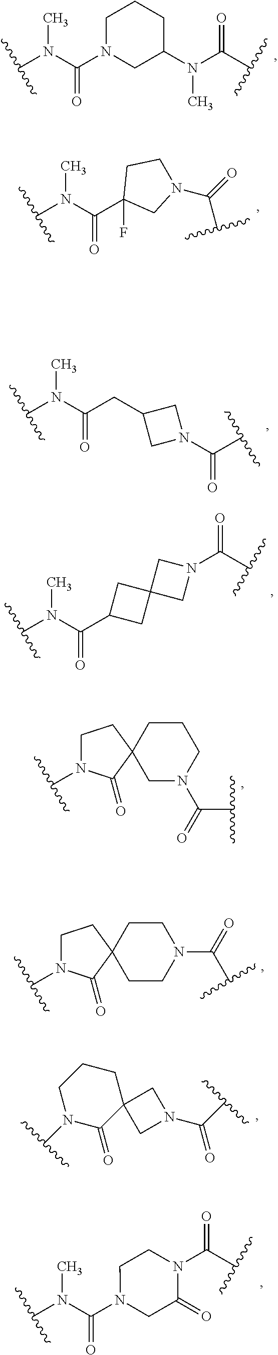

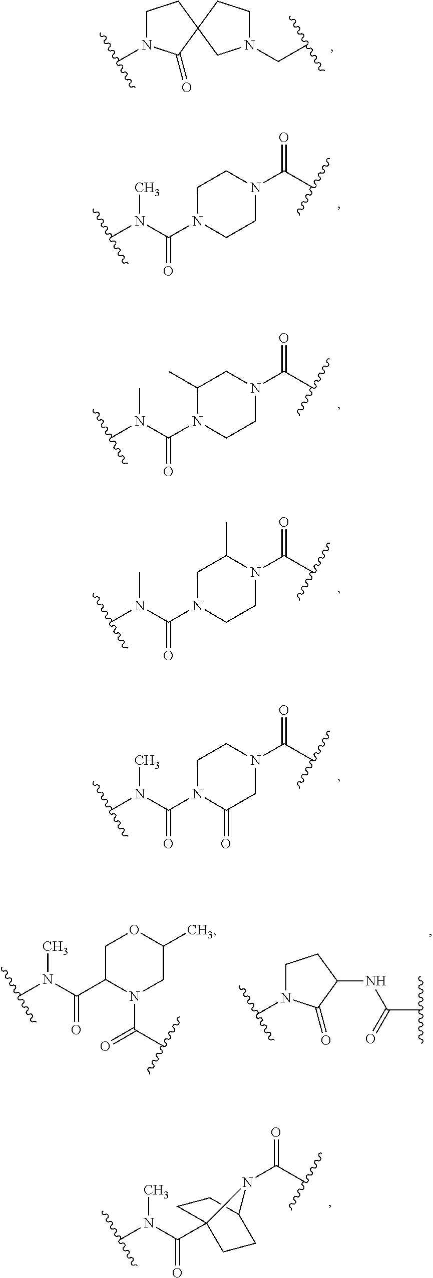

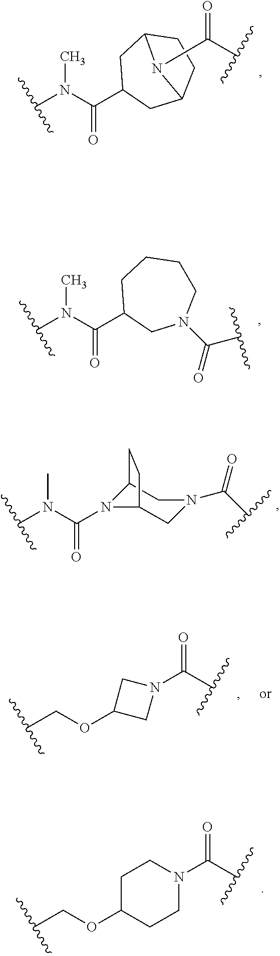 [SMILES: *C(=O)N1C2CCC1CC(C(=O)N(*)C)C2 ; *C(=O)N1CCCCC(C(=O)N(*)C)C1 ; *C(=O)N1CC2CCC(C1)N2C(=O)N(*)C ; *COC1CN(C(*)=O)C1 ; *COC1CCN(C(*)=O)CC1]

In some embodiments, the linker has the structure of Formula II-IIc:

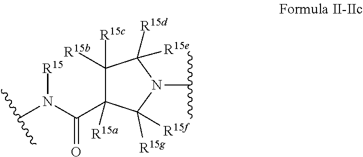

wherein R¹⁵ is hydrogen, optionally substituted C₁-C₆ alkyl, optionally substituted 3- to 8-membered cycloalkylene, or optionally substituted 3- to 8-membered heterocycloalkylene; and
R^{15a}, R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3- to 8-membered cycloalkylene, or optionally substituted 3- to 8-membered heterocycloalkylene.

In some embodiments, the linker has the structure:

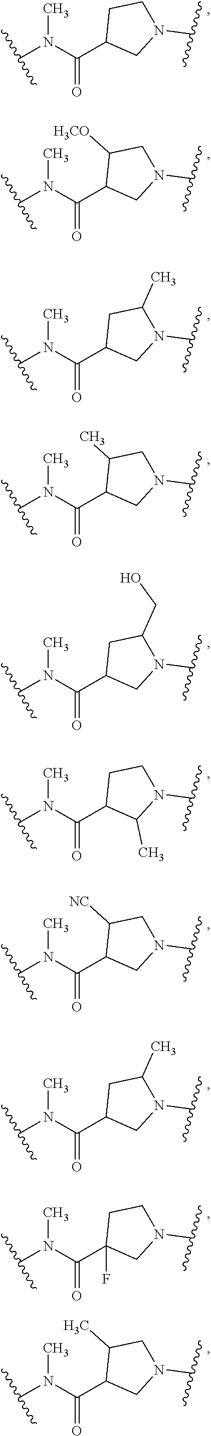

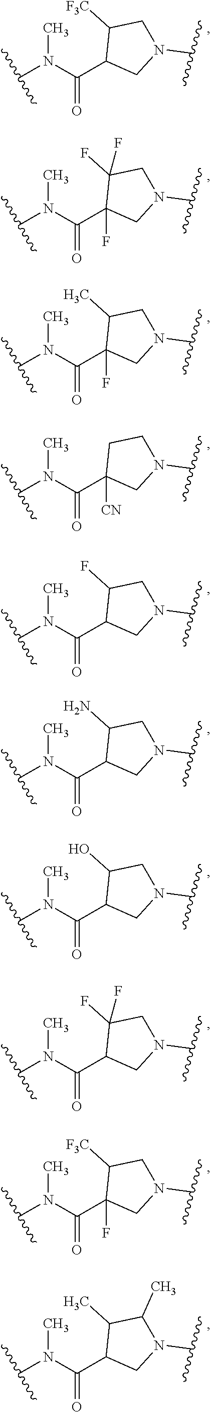

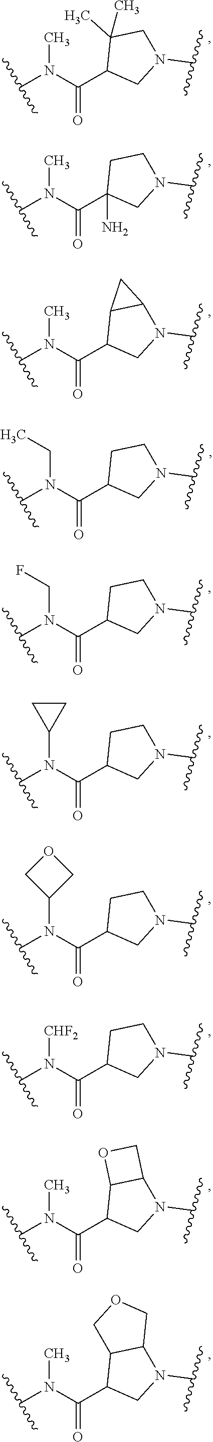

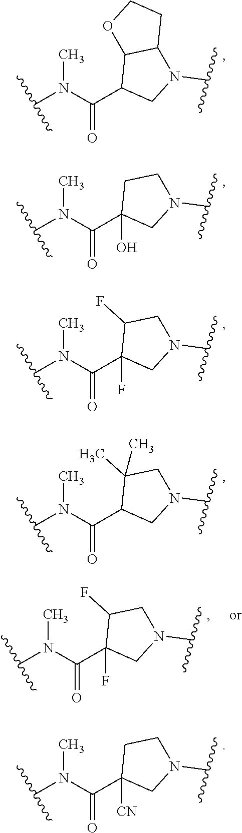

In some embodiments, the linker has the structure:

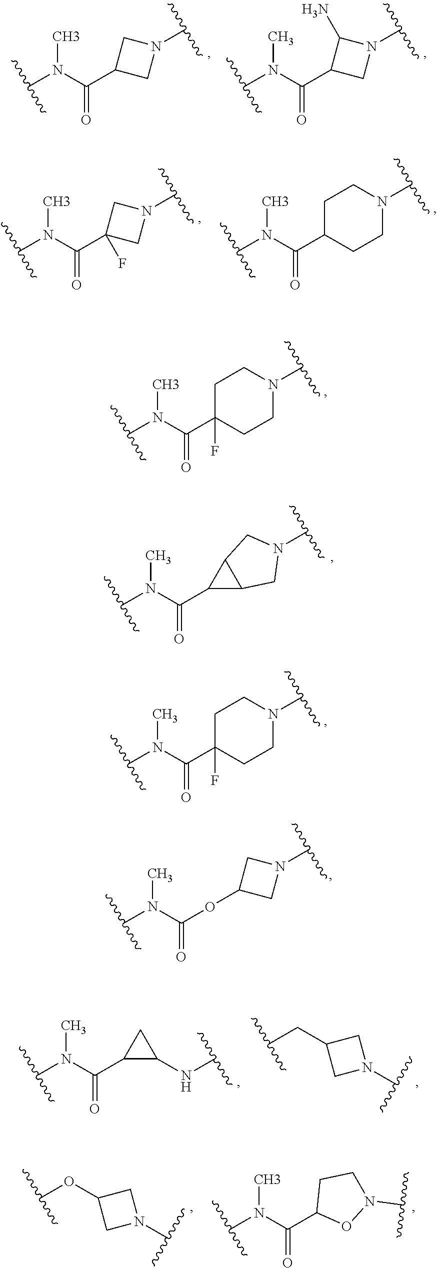

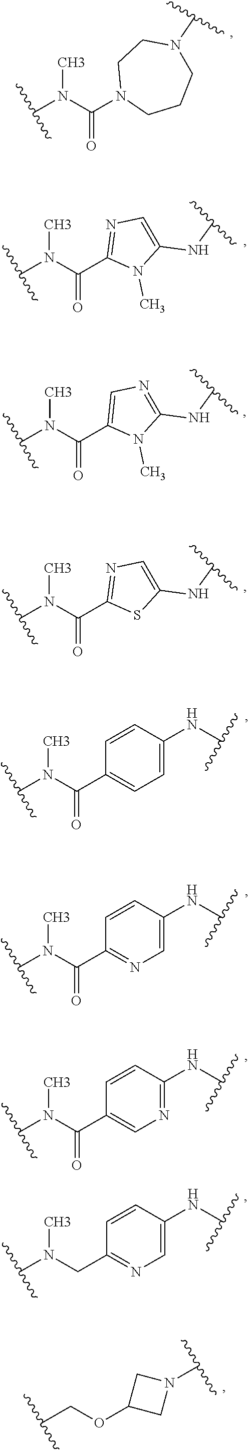

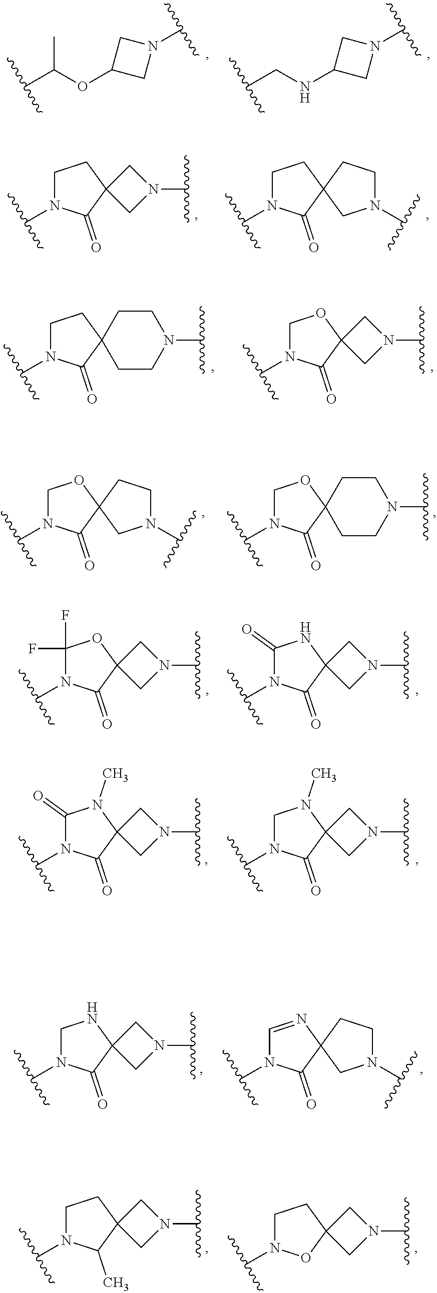

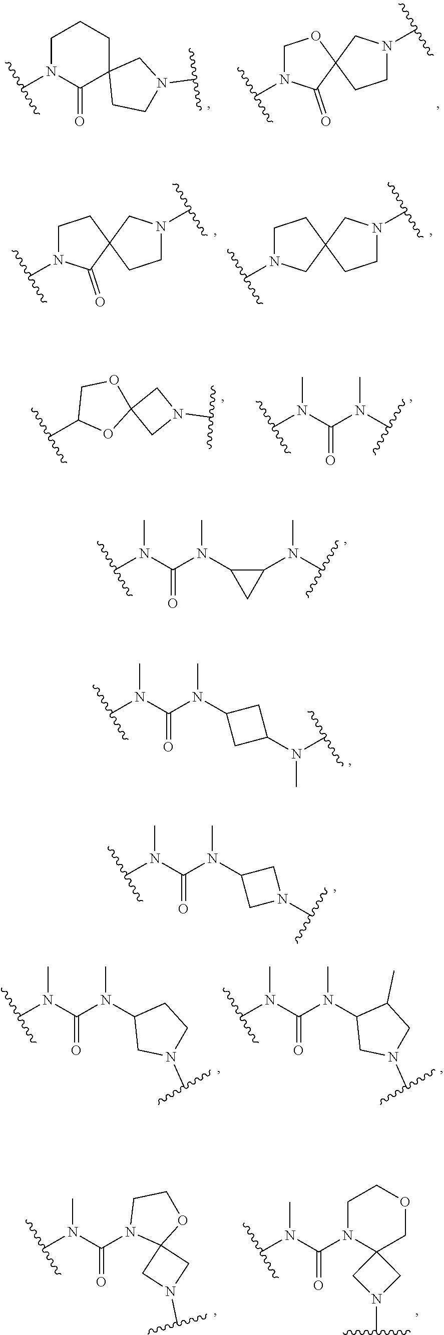

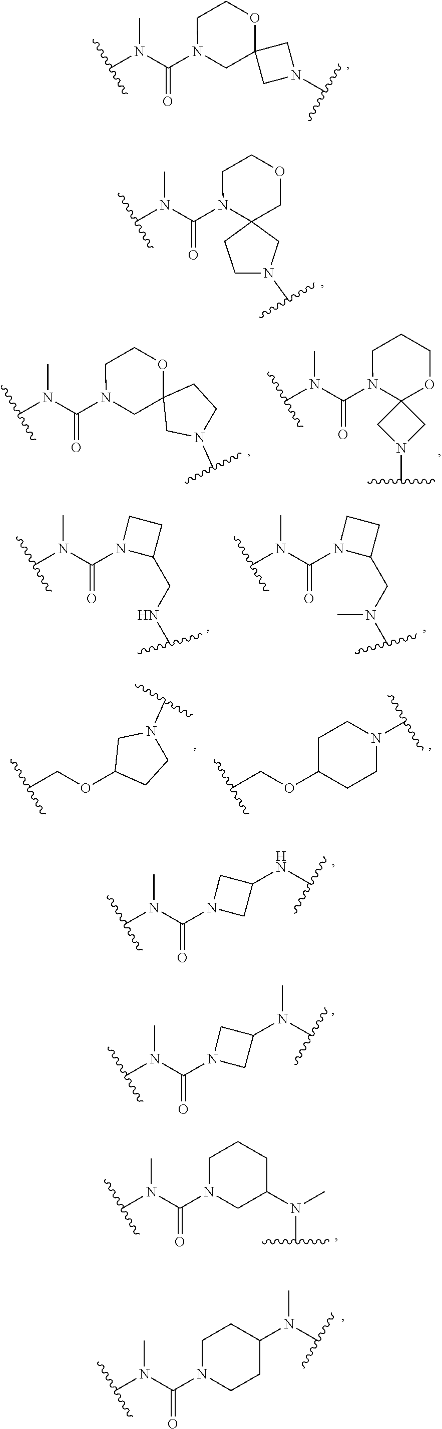

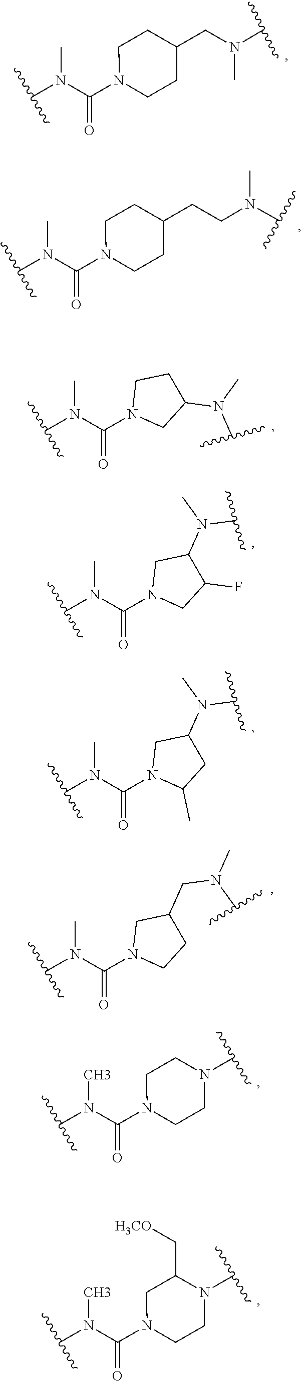

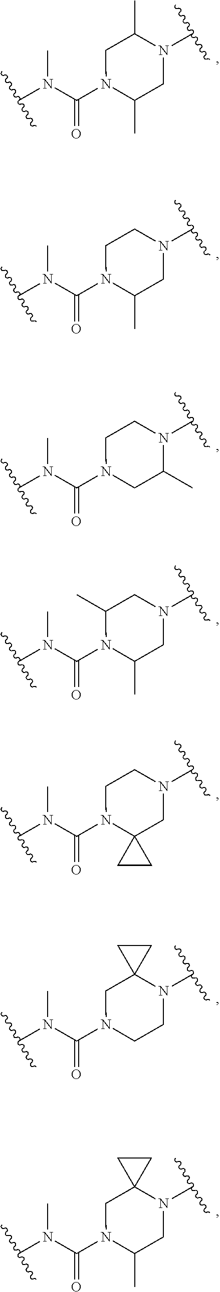

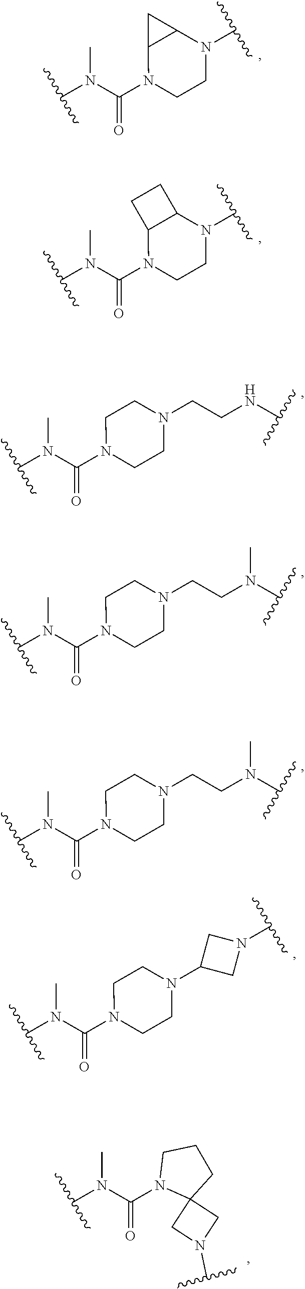

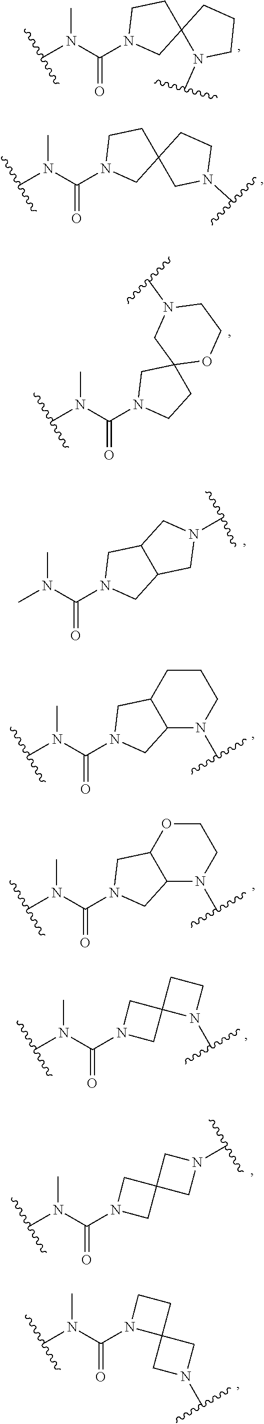

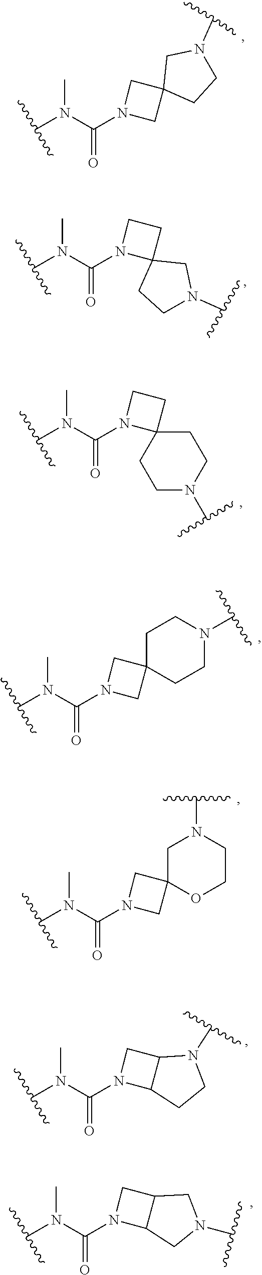

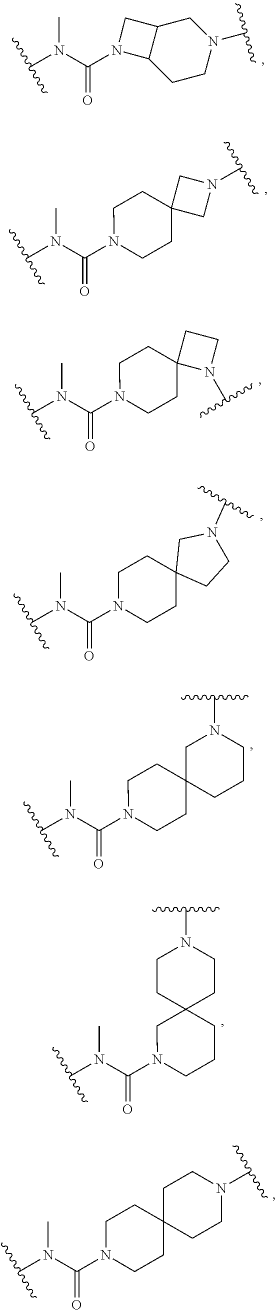

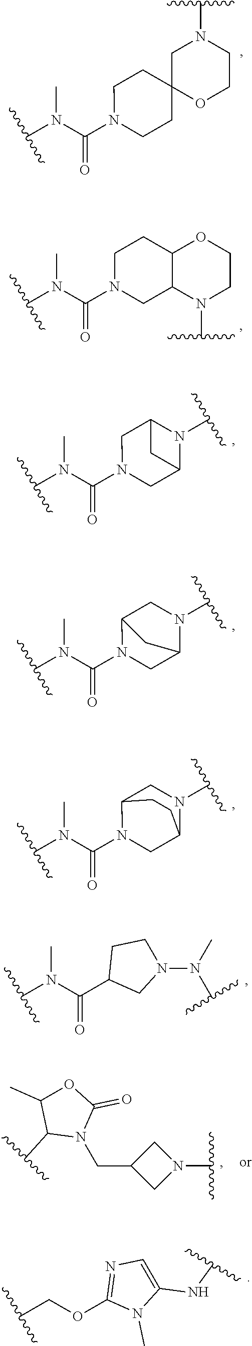

In some embodiments, the linker has the structure

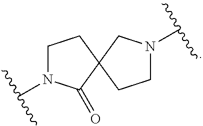 [SMILES: *N1CCC2(CCN(*)C2=O)C1]

In some embodiments, the linker has the structure

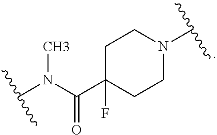 [SMILES: *N1CCC(F)(C(=O)N(*)C)CC1]

In some embodiments, a linker of Formula II is selected from the group consisting of

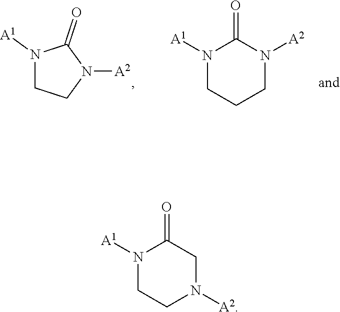

In an aspect, the invention features a compound having the structure of Formula IIIa or Formula IIIb:

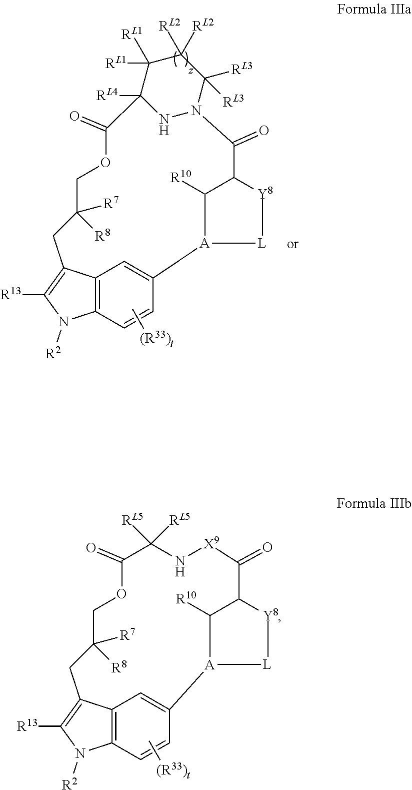

or pharmaceutically acceptable salt, an enantiomer, a stereoisomer, or a tautomer thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is

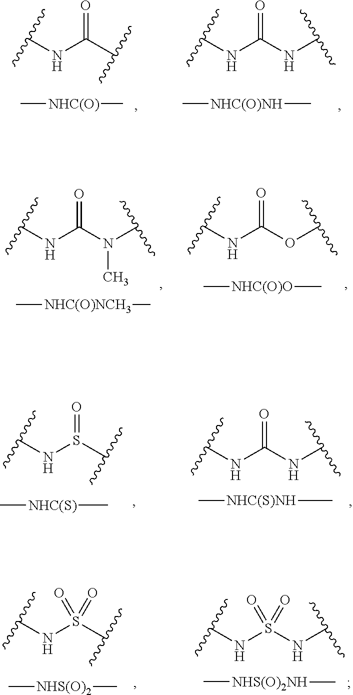

L is a linker;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl;
R⁷ and R⁸ are each, independently, selected from F or CH₃, or R⁷ and R⁸ combine with the atoms to which they are attached to make a 3-membered cycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
t is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In an aspect, the invention features a compound having the structure of Formula IIIa-1:

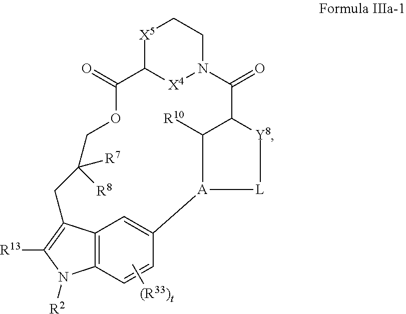

or pharmaceutically acceptable salt, an enantiomer, a stereoisomer, or a tautomer thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is

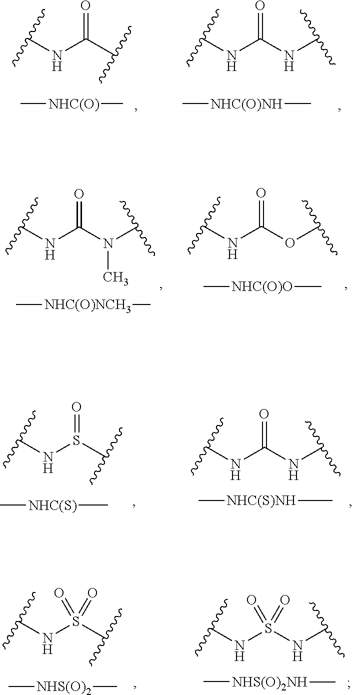

L is a linker;
X⁴ and X⁵ are each, independently, CH₂, CH(CH₃) or NH;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl;
R⁷ and R⁸ are each, independently, selected from F or CH₃, or R⁷ and R⁸ combine with the atoms to which they are attached to make a 3-membered cycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the disclosure features a compound of structural Formula IIIa-2:

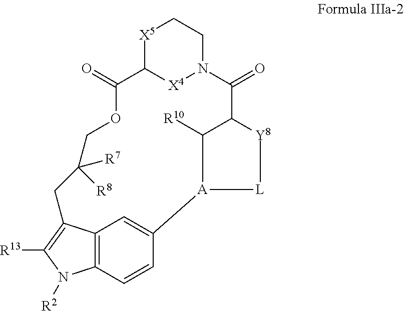

or pharmaceutically acceptable salt, an enantiomer, a stereoisomer, or a tautomer thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is

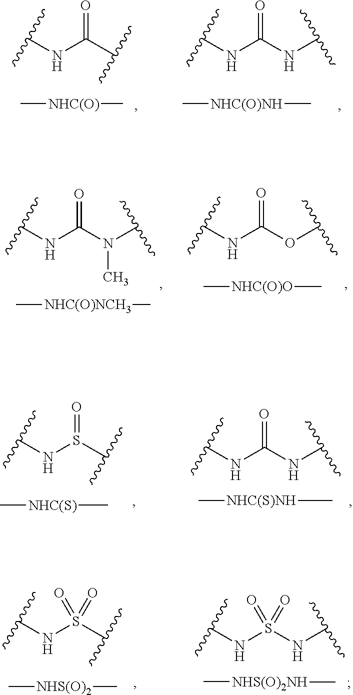

L is a linker;
X⁴ and X⁵ are each, independently, CH₂, CH(CH₃) or NH;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl; and
R⁷ and R⁸ are each, independently, selected from F or CH₃, or R⁷ and R⁸ combine with the atoms to which they are attached to make a 3-membered cycloalkyl.

In some embodiments, the compound has the structure of Formula IIIa-3:

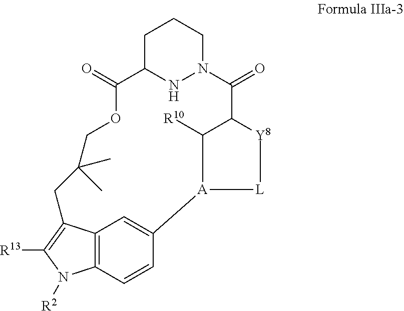

or pharmaceutically acceptable salt, an enantiomer, a stereoisomer, or a tautomer thereof, wherein A is optionally substituted 3- to 6-membered cycloalkylene, optionally substituted 3- to 6-membered heterocycloalkylene, optionally substituted 6-membered arylene, or optionally substituted 5- to 6-membered heteroarylene;
Y⁸ is

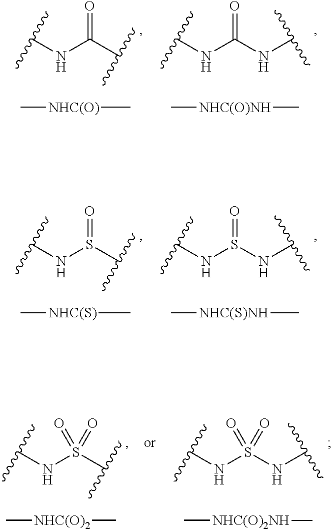

R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl; and
R¹⁰ is hydrogen or optionally substituted C₁-C₆ heteroalkyl. In some embodiments, R¹⁰ is hydrogen.

In some embodiments, R¹³ is optionally substituted 6- to 10-membered aryl or optionally substituted 5- to 10-membered heteroaryl. In some embodiments, R¹³ is optionally substituted phenyl or optionally substituted pyridine.

In some embodiments, A is optionally substituted thiazole, optionally substituted triazole, optionally substituted morpholino, optionally substituted piperidinyl, optionally substituted pyridine, or optionally substituted phenyl. In some embodiments, A is optionally substituted thiazole, optionally substituted triazole, optionally substituted morpholino, or phenyl. In some embodiments, A is not an optionally substituted phenyl or benzimidazole. In some embodiments, A is not hydroxyphenyl.

In some embodiments, Y⁸ is —NHC(O)— or —NHC(O)NH—.

In some embodiments, the compound has the structure of Formula IIIa-4:

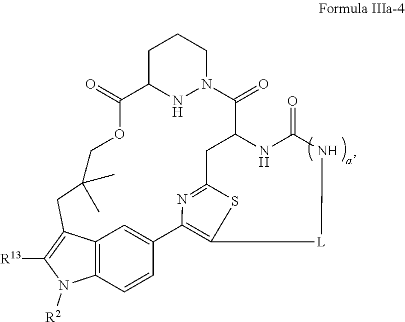

or pharmaceutically acceptable salt, an enantiomer, a stereoisomer, or a tautomer thereof, wherein a is 0 or 1.

In some embodiments, the compound has the structure of Formula IIIa-5:

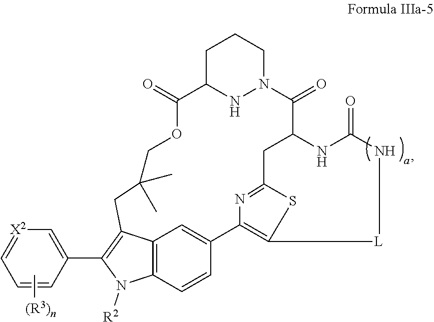

or pharmaceutically acceptable salt, an enantiomer, a stereoisomer, or a tautomer thereof, wherein X² is N or OH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula IIIa-6:

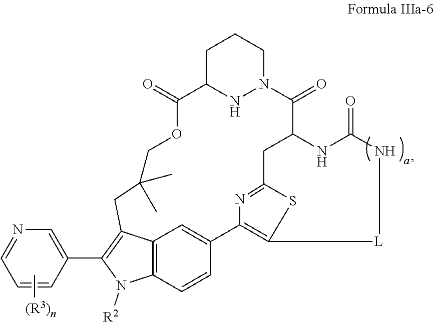

or pharmaceutically acceptable salt, an enantiomer, a stereoisomer, or a tautomer thereof.

In some embodiments, the compound has the structure of Formula IIIa-7:

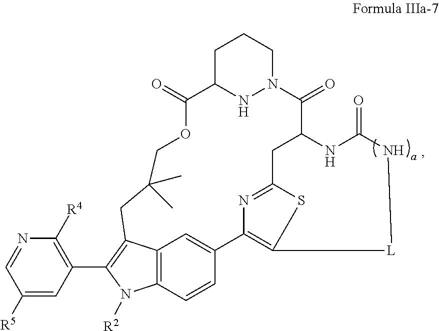

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula IIIa-8:

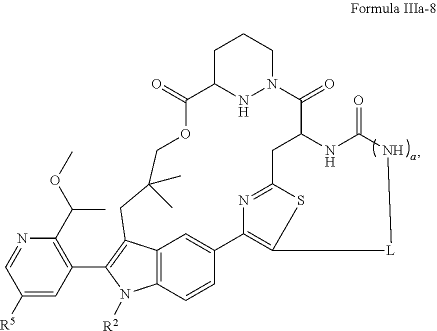

or pharmaceutically acceptable salt, an enantiomer, a stereoisomer, or a tautomer thereof.

In some embodiments, the compound has the structure of Formula IIIa-9:

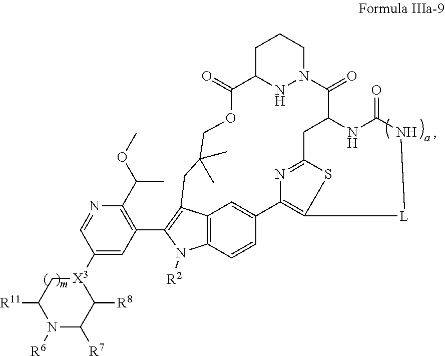

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is H. In some embodiments, X³ is N, m is 1, and R¹¹ is H.

In some embodiments, the compound has the structure of Formula IIIa-10:

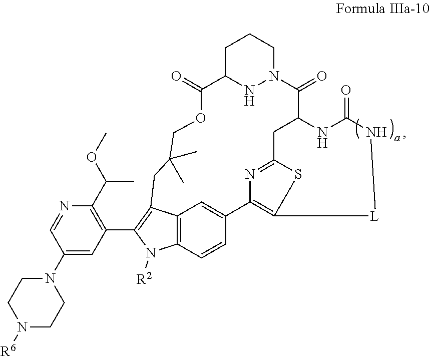

or pharmaceutically acceptable salt, an enantiomer, a stereoisomer, or a tautomer thereof.

In some embodiments, the compound has the structure of Formula IIIa-11:

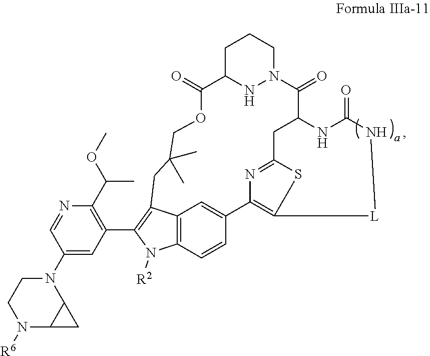

or pharmaceutically acceptable salt, an enantiomer, a stereoisomer, or a tautomer thereof.

In some embodiments (e.g., of any one of Formulae IIIa-10 or IIIa-11), R⁶ is methyl.

In some embodiments, the compound has the structure of Formula IIIa-12 or Formula IIIa-13:

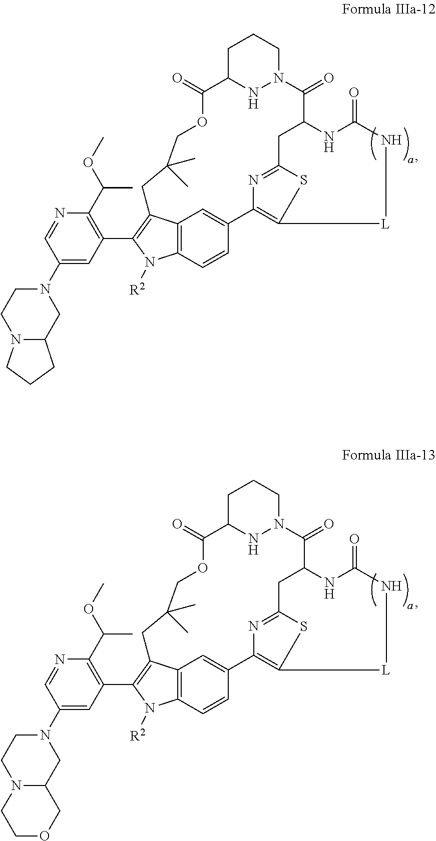

or pharmaceutically acceptable salt, an enantiomer, a stereoisomer, or a tautomer thereof.

In some embodiments, the compound has the structure of Formula IIIa-a:

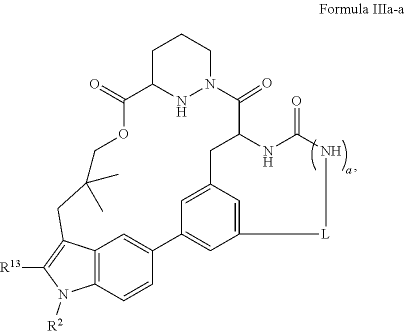

or pharmaceutically acceptable salt, an enantiomer, a stereoisomer, or a tautomer thereof, wherein a is 0 or 1.

In some embodiments, the compound has the structure of Formula IIIa-a1:

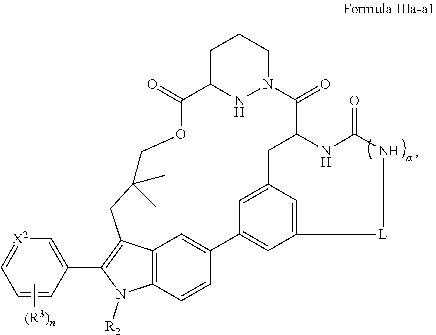

or pharmaceutically acceptable salt, an enantiomer, a stereoisomer, or a tautomer thereof, wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula IIIa-a2:

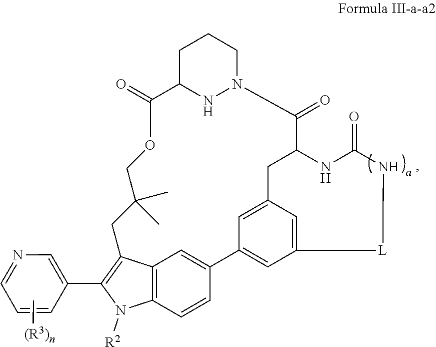

or pharmaceutically acceptable salt, an enantiomer, a stereoisomer, or a tautomer thereof.

In some embodiments, the compound has the structure of Formula IIIa-a3:

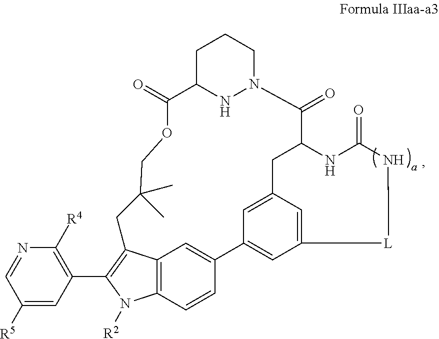

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula IIIa-a4:

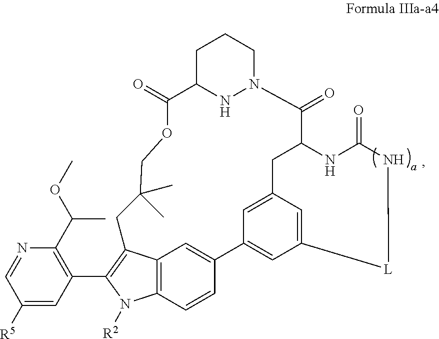

or pharmaceutically acceptable salt, an enantiomer, a stereoisomer, or a tautomer thereof.

In some embodiments, the compound has the structure of Formula IIIa-a5:

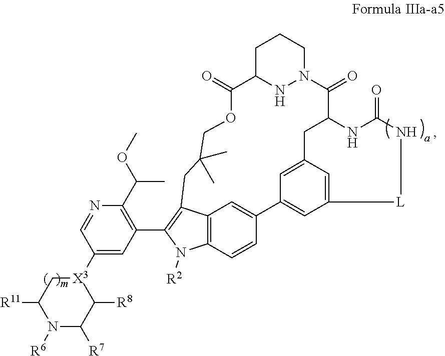

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound has the structure of Formula IIIa-a6:

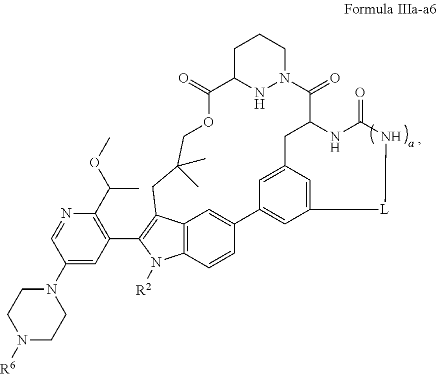

or pharmaceutically acceptable salt, an enantiomer, a stereoisomer, or a tautomer thereof.

In some embodiments, the compound has the structure of Formula IIIa-a7:

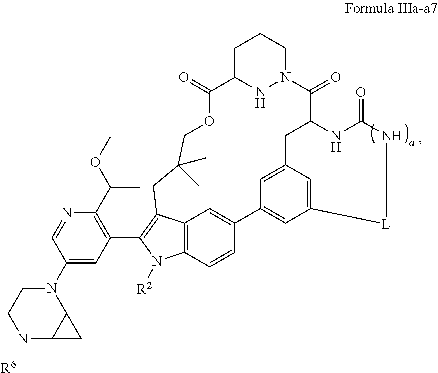

or pharmaceutically acceptable salt, an enantiomer, a stereoisomer, or a tautomer thereof.

In some embodiments (e.g., of any one of Formulae IIIa-a6 or IIIa-a7), R⁶ is methyl.

In some embodiments, the compound has the structure of Formula IIIa-a8 or Formula IIIa-a9:

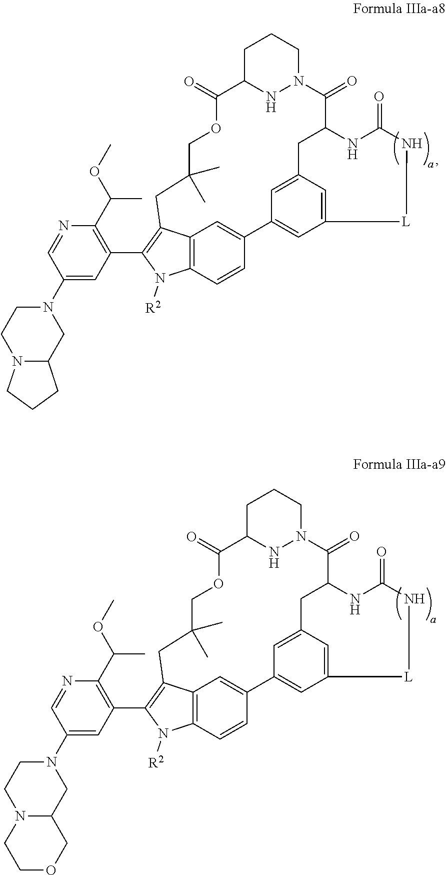

or pharmaceutically acceptable salt, an enantiomer, a stereoisomer, or a tautomer thereof.

In some embodiments, the compound has the structure of Formula III-IVa:

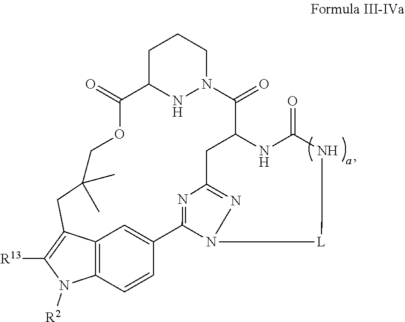

or pharmaceutically acceptable salt, an enantiomer, a stereoisomer, or a tautomer thereof, wherein a is 0 or 1.

In some embodiments, the compound has the structure of Formula III-IVa-1:

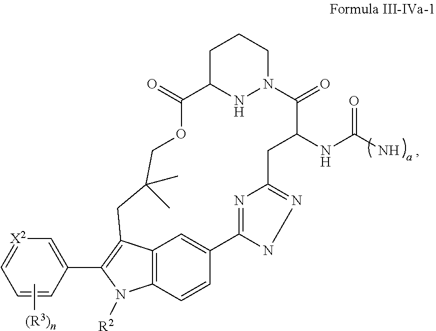

or pharmaceutically acceptable salt, an enantiomer, a stereoisomer, or a tautomer thereof, wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3 to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula III-IVa-2:

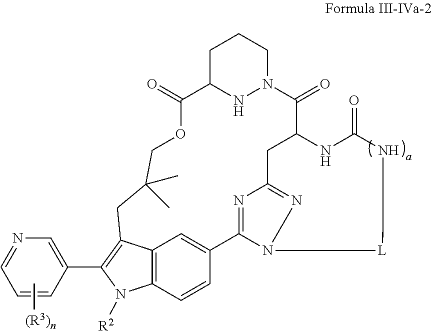

or pharmaceutically acceptable salt, an enantiomer, a stereoisomer, or a tautomer thereof.

In some embodiments, the compound has the structure of Formula III-Iva-3:

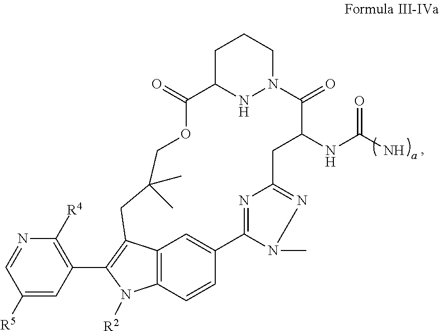

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula III-IVa-4:

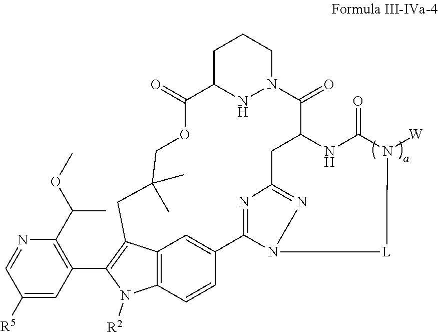

or pharmaceutically acceptable salt, an enantiomer, a stereoisomer, or a tautomer thereof.

In some embodiments, the compound has the structure of Formula III-IVa-5:

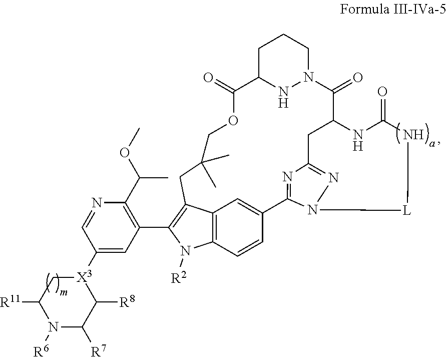

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound has the structure of Formula III-IVa-6:

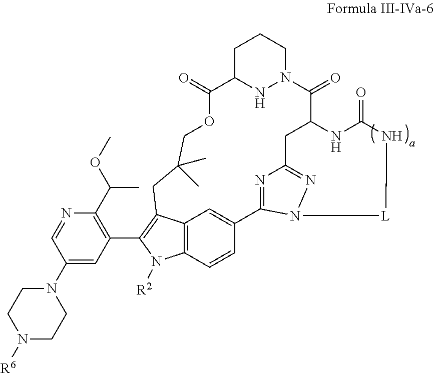

or pharmaceutically acceptable salt, an enantiomer, a stereoisomer, or a tautomer thereof.

In some embodiments, the compound has the structure of Formula III-IVa-7:

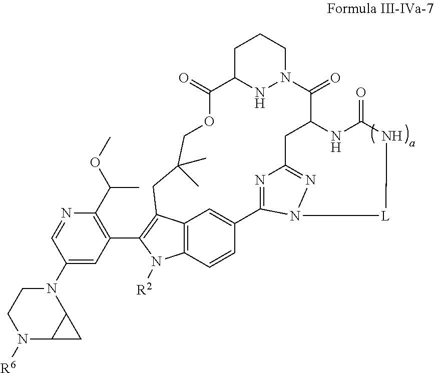

or pharmaceutically acceptable salt, an enantiomer, a stereoisomer, or a tautomer thereof.

In some embodiments (e.g., of any one of Formulae III-IVa-6 or III-IVa-7), R⁶ is methyl.

In some embodiments, the compound has the structure of Formula III-IVa-8 or Formula III-IVa-9:

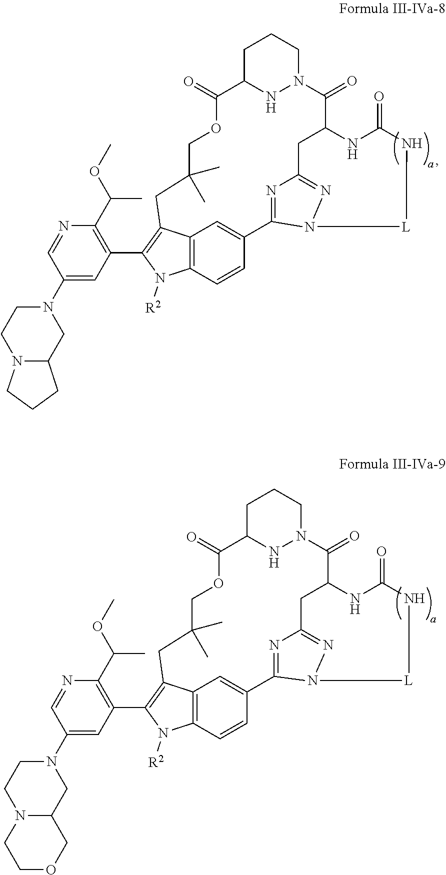

or pharmaceutically acceptable salt, an enantiomer, a stereoisomer, or a tautomer thereof.

In some embodiments, Y⁸ is —NHS(O)₂— or —NHS(O)₂NH—.

In some embodiments, the compound has the structure of Formula III-Va:

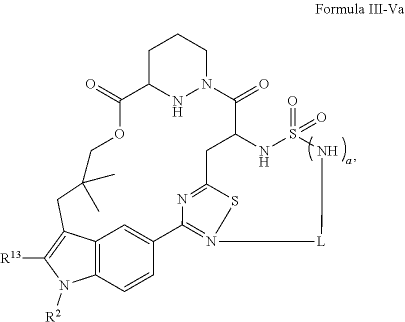

or pharmaceutically acceptable salt, an enantiomer, a stereoisomer, or a tautomer thereof, wherein a is 0 or 1.

In some embodiments, the compound has the structure of Formula III-Va-1:

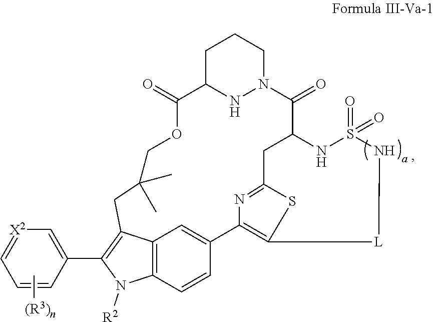

or pharmaceutically acceptable salt, an enantiomer, a stereoisomer, or a tautomer thereof, wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula III-Va-2:

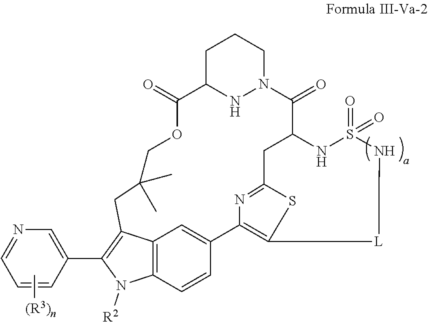

or pharmaceutically acceptable salt, an enantiomer, a stereoisomer, or a tautomer thereof.

In some embodiments, the compound has the structure of Formula III-Va-3:

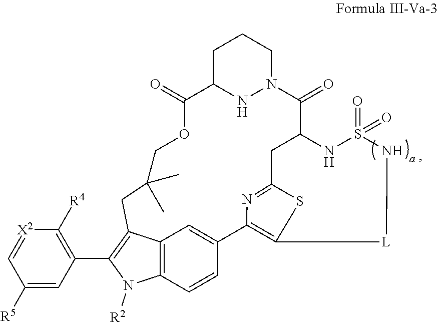

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula III-Va-4:

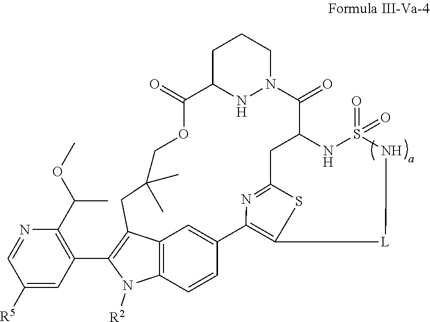

or pharmaceutically acceptable salt, an enantiomer, a stereoisomer, or a tautomer thereof.

In some embodiments, the compound has the structure of Formula III-Va-5:

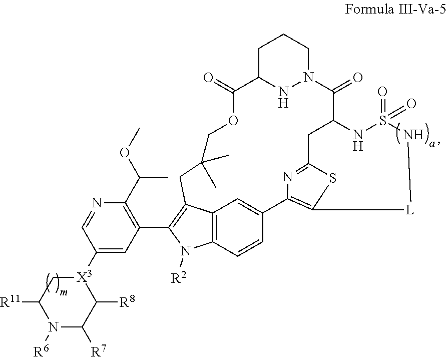

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound has the structure of Formula III-Via:

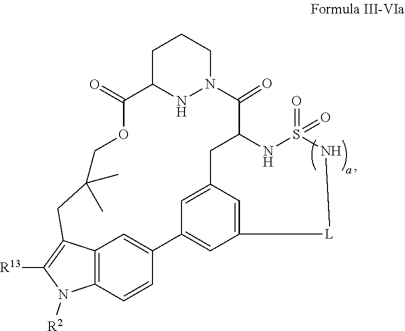

or pharmaceutically acceptable salt, an enantiomer, a stereoisomer, or a tautomer thereof, wherein a is 0 or 1.

In some embodiments, the compound has the structure of Formula III-Via-1:

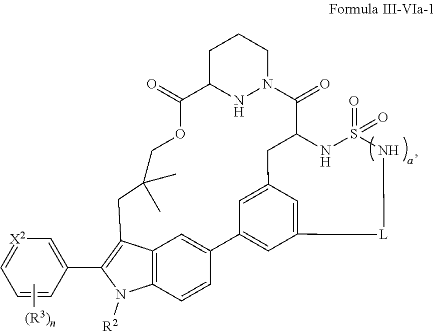

or pharmaceutically acceptable salt, an enantiomer, a stereoisomer, or a tautomer thereof, wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula III-VIa-2:

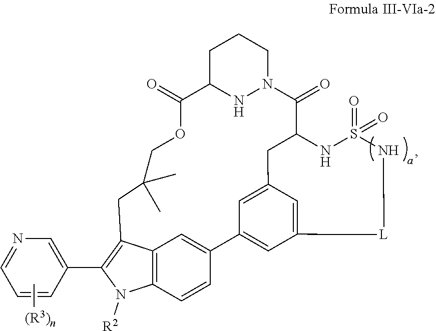

or pharmaceutically acceptable salt, an enantiomer, a stereoisomer, or a tautomer thereof.

In some embodiments, the compound has the structure of Formula III-VIa-3:

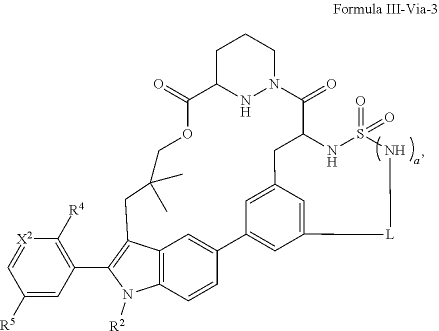

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula III-Via-4:

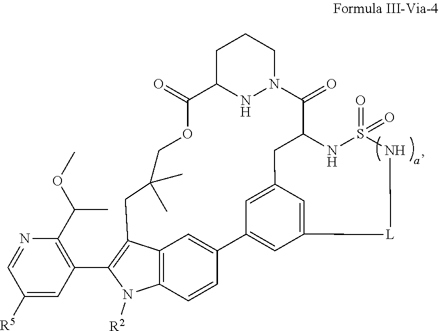

or pharmaceutically acceptable salt, an enantiomer, a stereoisomer, or a tautomer thereof.

In some embodiments, the compound has the structure of Formula III-VIa-5:

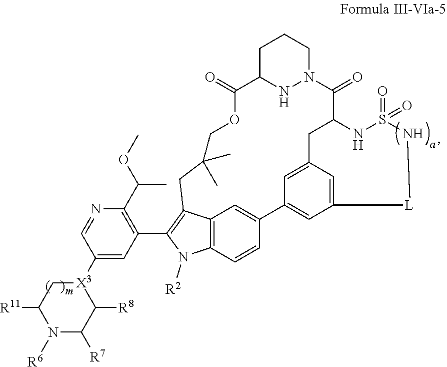

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound has the structure of Formula III-VIIa:

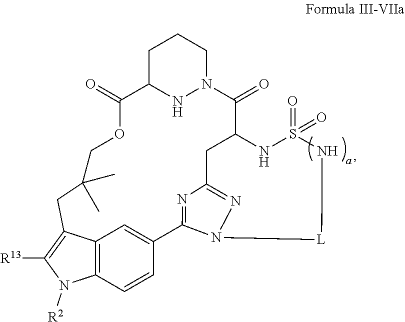

or pharmaceutically acceptable salt, an enantiomer, a stereoisomer, or a tautomer thereof, wherein R⁹ is H or C₁-C₆ alkyl; and
a is 0 or 1.

In some embodiments, the compound has the structure of Formula III-VIIa-1:

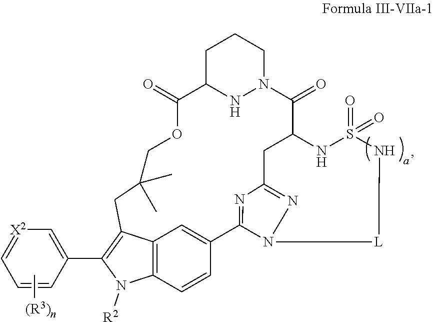

or pharmaceutically acceptable salt, an enantiomer, a stereoisomer, or a tautomer thereof, wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula III-VIIa-2:

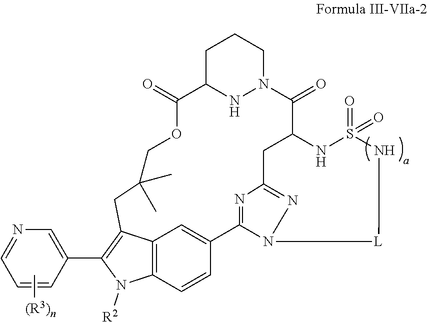

or pharmaceutically acceptable salt, an enantiomer, a stereoisomer, or a tautomer thereof.

In some embodiments, the compound has the structure of Formula III-VIIa-3:

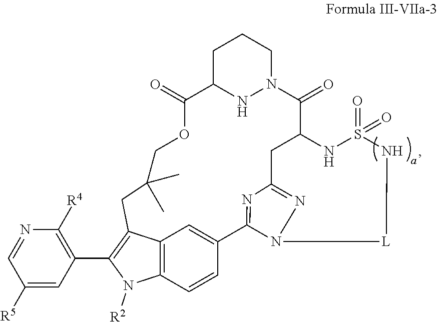

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula III-VIIa-4:

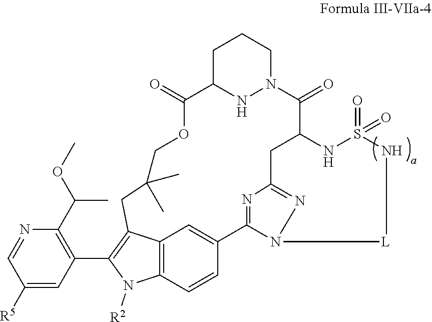

or pharmaceutically acceptable salt, an enantiomer, a stereoisomer, or a tautomer thereof.

In some embodiments, the compound has the structure of Formula III-VIIa-5:

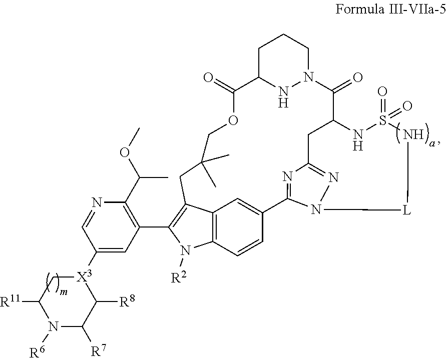

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae VIIa, VIIa-1, VIIa-2, VIIa-3, VIIa-4, or VIIa-5), R⁹ is methyl.

In some embodiments, Y is —NHS(O)— or —NHS(O)NH—.

In some embodiments, the compound has the structure of Formula III-VIIIa:

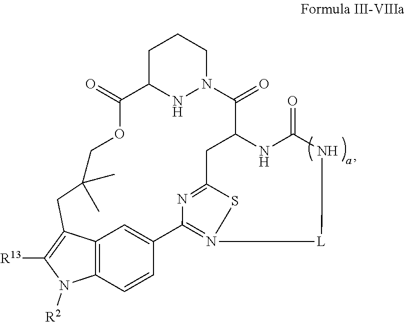

or pharmaceutically acceptable salt, an enantiomer, a stereoisomer, or a tautomer thereof, wherein a is 0 or 1.

In some embodiments, the compound has the structure of Formula III-VIIIa-1:

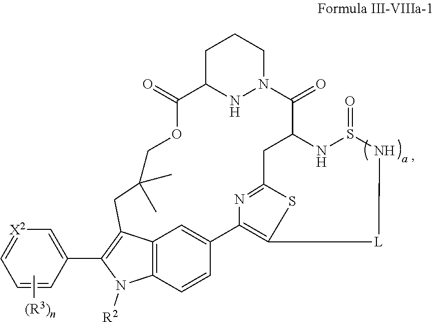

or pharmaceutically acceptable salt, an enantiomer, a stereoisomer, or a tautomer thereof, wherein X² is N or OH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula III-VIIIa-2:

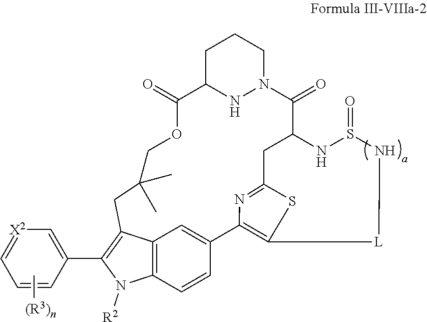

or pharmaceutically acceptable salt, an enantiomer, a stereoisomer, or a tautomer thereof.

In some embodiments, the compound has the structure of Formula III-VIIIa-3:

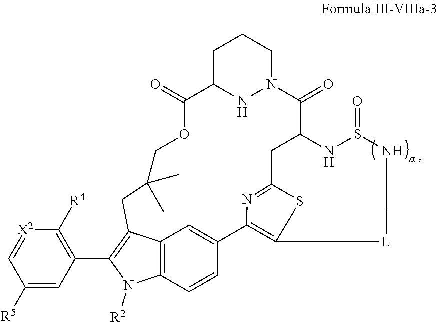

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula III-VIIIa-4:

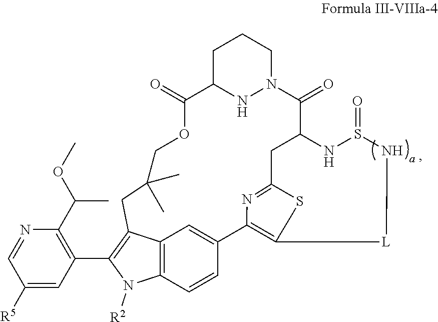

or pharmaceutically acceptable salt, an enantiomer, a stereoisomer, or a tautomer thereof.

In some embodiments, the compound has the structure of Formula III-VIIIa-5:

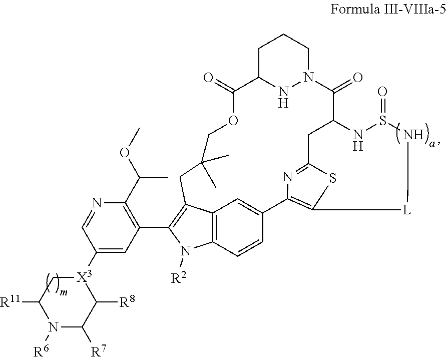

or pharmaceutically acceptable salt, an enantiomer, a stereoisomer, or a tautomer thereof, wherein X³ is N or OH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound has the structure of Formula III-IXa:

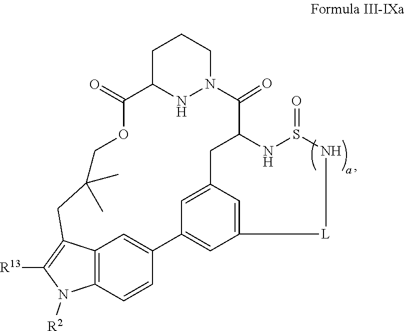

or pharmaceutically acceptable salt, an enantiomer, a stereoisomer, or a tautomer thereof, wherein a is 0 or 1.

In some embodiments, the compound has the structure of Formula III-IXa-1:

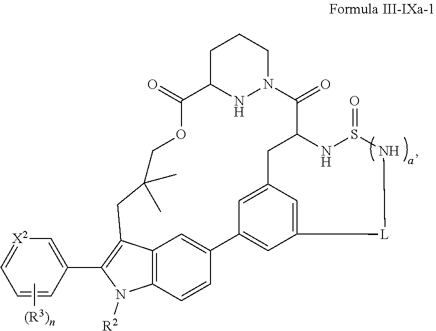

or pharmaceutically acceptable salt, an enantiomer, a stereoisomer, or a tautomer thereof, wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula III-IXa-2:

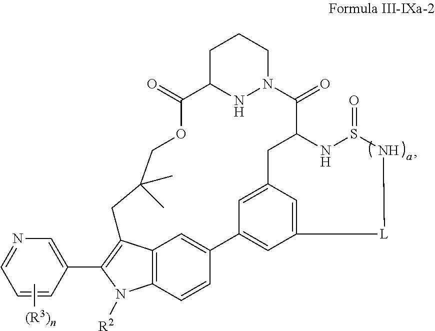

or pharmaceutically acceptable salt, an enantiomer, a stereoisomer, or a tautomer thereof.

In some embodiments, the compound has the structure of Formula III-IXa-3:

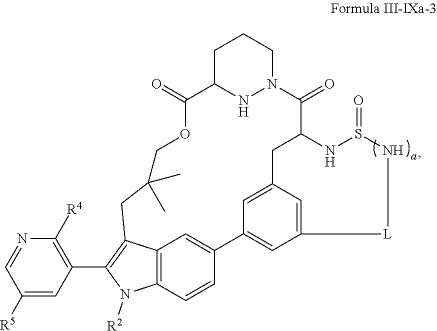

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula III-IXa-4:

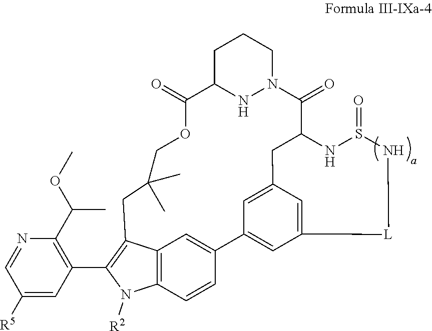

or pharmaceutically acceptable salt, an enantiomer, a stereoisomer, or a tautomer thereof.

In some embodiments, the compound has the structure of Formula III-IXa-5:

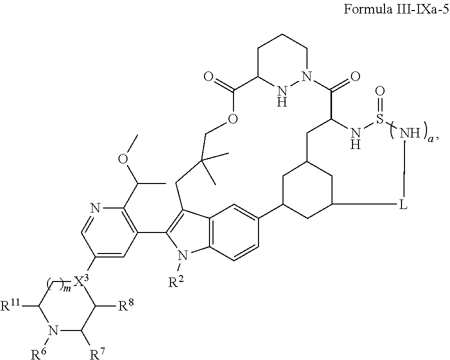

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
   R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the compound has the structure of Formula III-Xa:

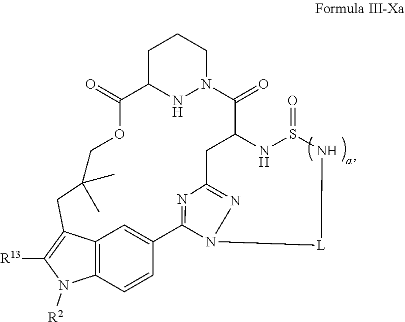

or pharmaceutically acceptable salt, an enantiomer, a stereoisomer, or a tautomer thereof, wherein a is 0 or 1.

In some embodiments, the compound has the structure of Formula III-Xa-1:

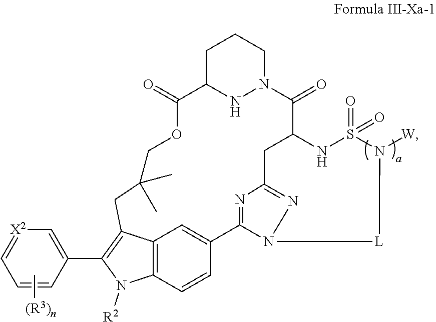

or pharmaceutically acceptable salt, an enantiomer, a stereoisomer, or a tautomer thereof, wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl; and
n is an integer from 1 to 4.

In some embodiments, the compound has the structure of Formula III-Xa-2:

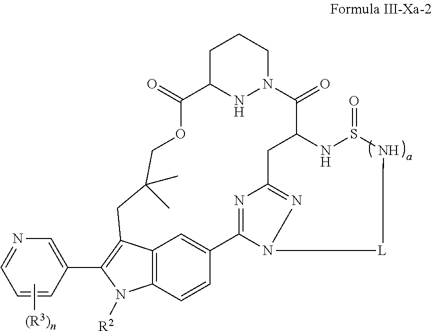

or pharmaceutically acceptable salt, an enantiomer, a stereoisomer, or a tautomer thereof.

In some embodiments, the compound has the structure of Formula III-Xa-3:

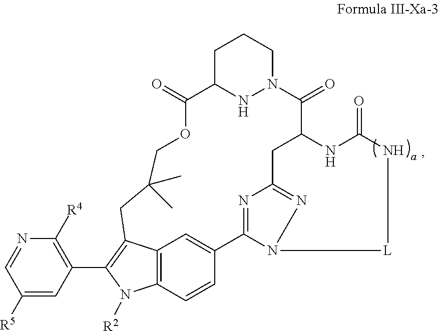

or pharmaceutically acceptable salt, an enantiomer, a stereoisomer, or a tautomer thereof, wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 11-membered heterocycloalkyl (e.g., optionally substituted 3- to 6-membered heterocycloalkyl), optionally substituted 6- to 10-membered aryl, or optionally substituted 5- to 10-membered heteroaryl.

In some embodiments, the compound has the structure of Formula III-Xa-4:

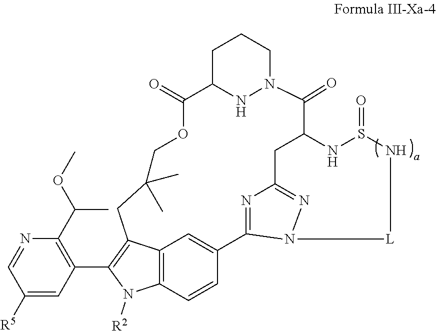

or pharmaceutically acceptable salt, an enantiomer, a stereoisomer, or a tautomer thereof.

In some embodiments, the compound has the structure of Formula III-Xa-5:

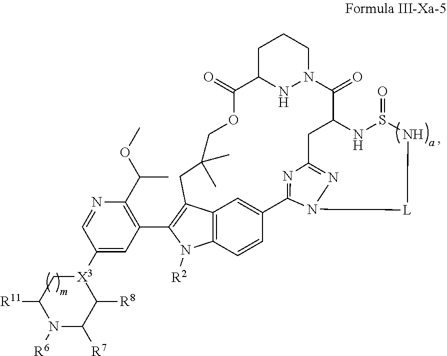

or pharmaceutically acceptable salt, an enantiomer, a stereoisomer, or a tautomer thereof, wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl; or
R⁶ and R⁷ combine with the atoms to which they are attached to form an optionally substituted 3- to 8-membered cycloalkyl or an optionally substituted 3- to 8-membered heterocycloalkyl; or
R⁷ and R⁸ combine with the atoms to which they are attached to form an optionally substituted 3- to 8-membered heterocycloalkyl; or
R⁷ and R¹¹ combine with the atoms to which they are attached to form an optionally substituted 4- to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments of any aspect described herein, a is 0. In some embodiments of any of the above, a is 0.

In some embodiments of any aspect described herein, R² is optionally substituted C₁-C₆ alkyl. In some embodiments, R² is selected from —CH₂CH₃ or —CH₂CF₃.

In an aspect, the invention features a compound having the structure of Formula IVa or Formula IVb:

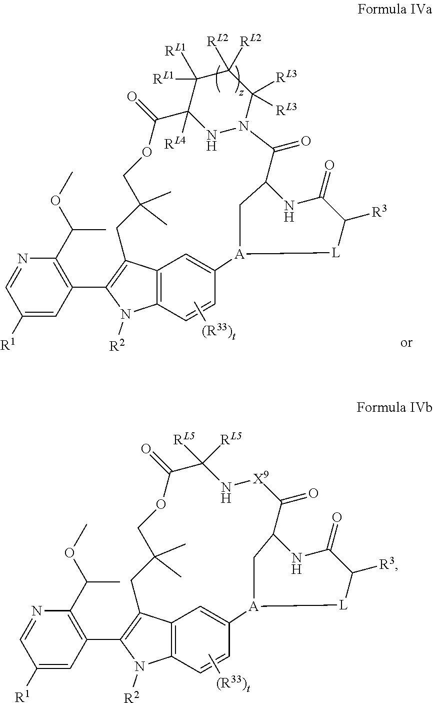

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the compound has the structure of Formula IVa-1 or Formula IVb-1:

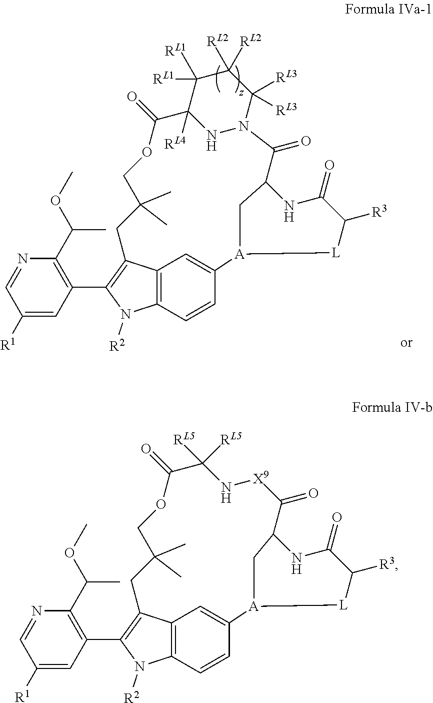

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In some embodiments, the compound has the structure of Formula IVa-2:

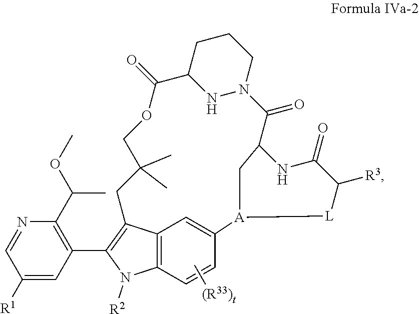

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the disclosure features a compound of structural Formula IVa-3:

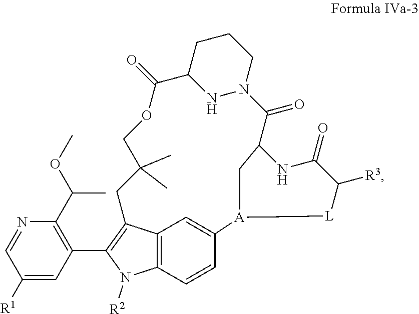

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

In some embodiments, provided herein is a compound having the structure of Formula IVa-4:

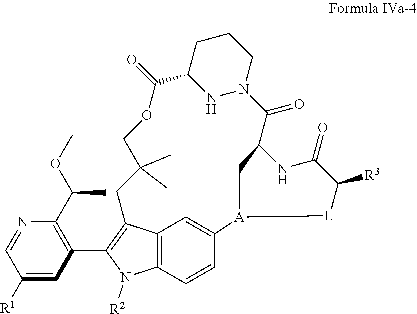

or pharmaceutically acceptable salt, an enantiomer, a stereoisomer, or a tautomer thereof.

In some embodiments of compounds of the present invention, A is optionally substituted thiazole, optionally substituted oxazole, optionally substituted morpholino, optionally substituted pyrrolidinyl, optionally substituted pyridyl, optionally substituted azetidinyl, optionally substituted pyrazinyl, optionally substituted pyrimidine, optionally substituted piperidinyl, optionally substituted oxadiazole, optionally substituted thiadiazole, optionally substituted triazole, optionally substituted thiomorpholino, or optionally substituted phenyl.

In some embodiments, the disclosure features a compound of structural Formula IVa5:

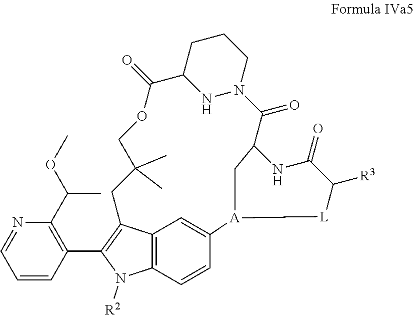

or pharmaceutically acceptable salt, an enantiomer, a stereoisomer, or a tautomer thereof.

In some embodiments, a compound having the structure of Formula IVa6 is provided:

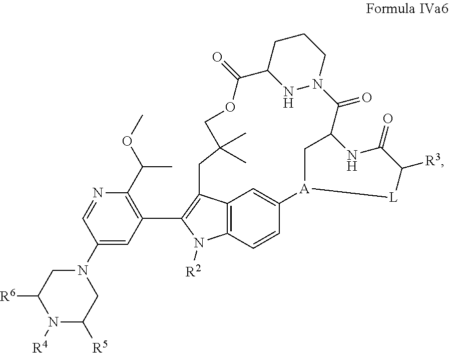

or pharmaceutically acceptable salt, an enantiomer, a stereoisomer, or a tautomer thereof, wherein R⁴, R⁵, and R⁶ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered heterocycloalkyl; or
R⁴ and R⁵ combine with the atoms to which they are attached to form an optionally substituted 3- to 8-membered cycloalkyl or an optionally substituted 3- to 8-membered heterocycloalkyl; or
R⁴ and R⁶ combine with the atoms to which they are attached to form an optionally substituted 3- to 8-membered cycloalkyl or an optionally substituted 3- to 8-membered heterocycloalkyl.

In some embodiments, a compound of the present invention has the structure of Formula IVa7:

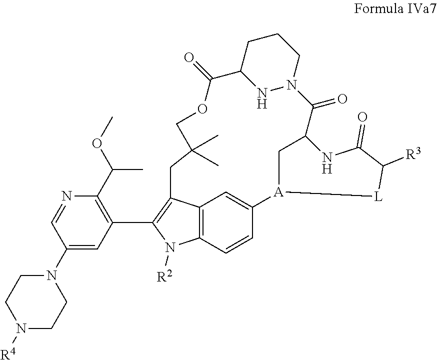

or pharmaceutically acceptable salt, an enantiomer, a stereoisomer, or a tautomer thereof.

In some embodiments, a compound of the present invention has the structure of Formula IVa8:

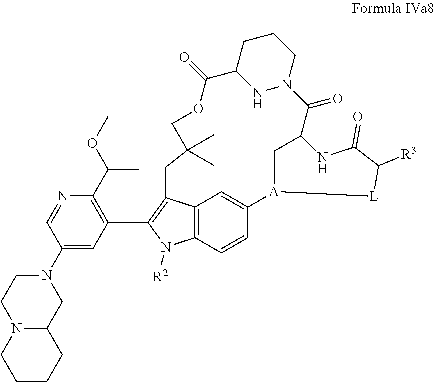

or pharmaceutically acceptable salt, an enantiomer, a stereoisomer, or a tautomer thereof.

In some embodiments, a compound of the present invention has the structure of Formula IVa9f:

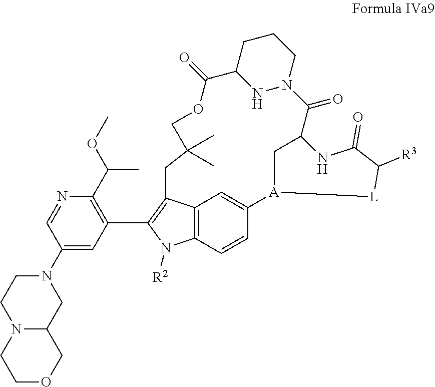

or pharmaceutically acceptable salt, an enantiomer, a stereoisomer, or a tautomer thereof.

In some embodiments of a compound of the present invention, R² is:

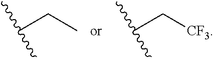

In some embodiments of a compound of the present invention, R³ is optionally substituted C₁-C₃ alkyl. In some embodiments, R³ is:

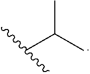 [SMILES: *C(C)C]

In some embodiments of a compound of the present invention, R³ is optionally substituted C₁-C₃ heteroalkyl. In some embodiments, R³ is:

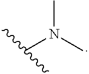 [SMILES: *N(C)C]

In some embodiments of a compound of the present invention, A is optionally substituted 5- to 10-membered heteroarylene. In some embodiments, A is:

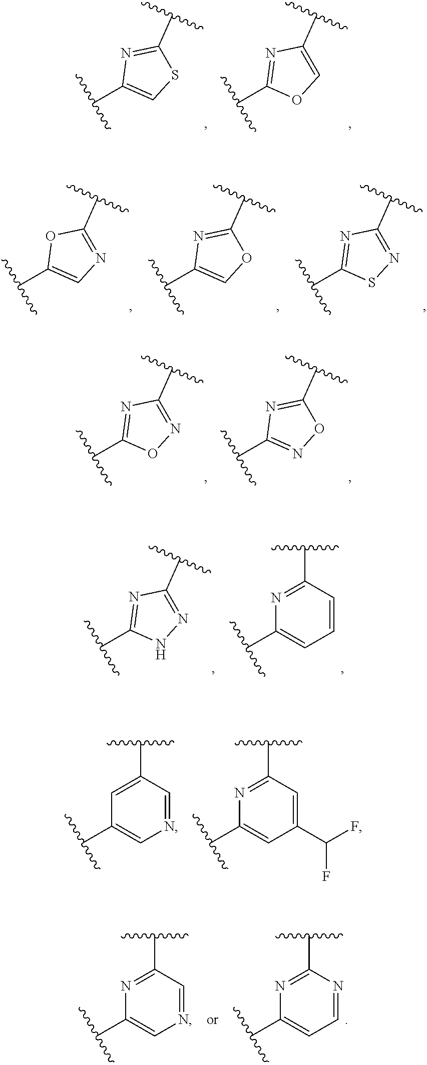

In some embodiments of a compound of the present invention, A is optionally substituted phenyl. In some embodiments, A is:

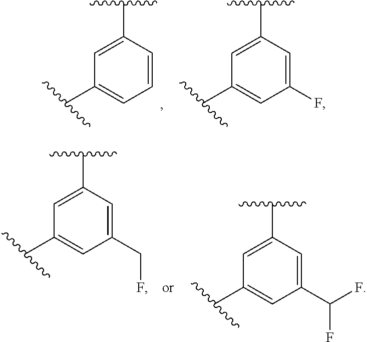 [SMILES: *c1cccc(*)c1 ; *c1cc(*)cc(F)c1 ; *c1cc(*)cc(CF)c1 ; *c1cc(*)cc(C(F)F)c1]

In some embodiments of a compound of the present invention, A is optionally substituted 3- to 6-membered heterocycloalkylene. In some embodiments, A is selected from the following, or a stereoisomer thereof:

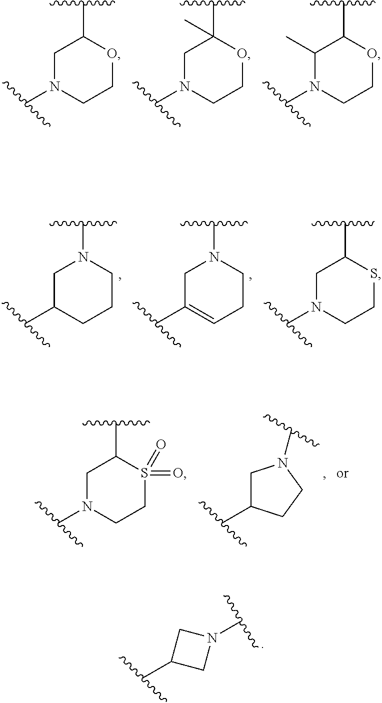

In some embodiments of a compound of the present invention, the linker is the structure of Formula IV-III:

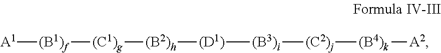

wherein A¹ is a bond between the linker and CH(R³); A² is a bond between A and the linker; B¹, B², B³, and B⁴ each, independently, is selected from optionally substituted C₁-C₂ alkylene, optionally substituted C₁-C₃ heteroalkylene, O, S, and NR^{N}; each R^{N} is, independently, hydrogen, optionally substituted C₁-C₄ alkyl, optionally substituted C₂-C₄ alkenyl, optionally substituted C₂-C₄ alkynyl, optionally substituted 3- to 14-membered heterocycloalkyl, optionally substituted 6- to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3- to 14-membered heterocycloalkylene, optionally substituted 5- to 10-membered heteroarylene, optionally substituted 3- to 8-membered cycloalkylene, optionally substituted 6- to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A².

In some embodiments of a compound of the present invention, the linker is or comprises a cyclic moiety. In some embodiments, the linker has the structure of Formula IV-IIIa:

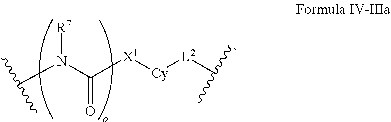

wherein o is 0 or 1;
R⁷ is hydrogen, optionally substituted C₁-C₆ alkyl, optionally substituted 3- to 8-membered cycloalkylene, or optionally substituted 3- to 8-membered heterocycloalkylene;
X¹ is absent, optionally substituted C₁-C₄ alkylene, O, NCH₃, or optionally substituted C₁-C₄ heteroalkylene;
Cy is optionally substituted 3- to 8-membered cycloalkylene, optionally substituted 3- to 12-membered heterocycloalkylene, optionally substituted 6- to 10-membered arylene, or optionally substituted 5- to 10-membered heteroarylene; and
L² is absent, —SO₂—, —NH—, optionally substituted C₁-C₄ alkylene, optionally substituted C₁-C₄ heteroalkylene, or optionally substituted 3- to 6-membered heterocycloalkylene.

In some embodiments, the linker is selected from, or a stereoisomer thereof:

[chemical structure]

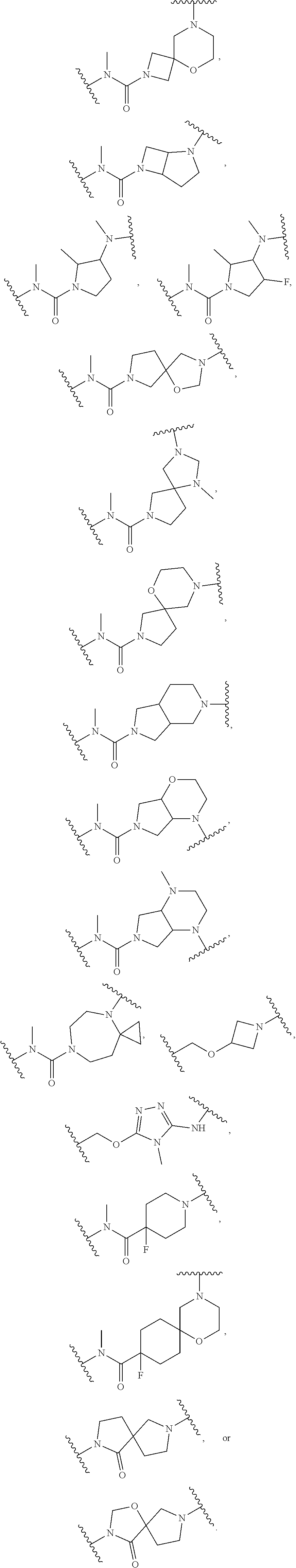

In some embodiments, the linker is selected from, or a stereoisomer thereof:

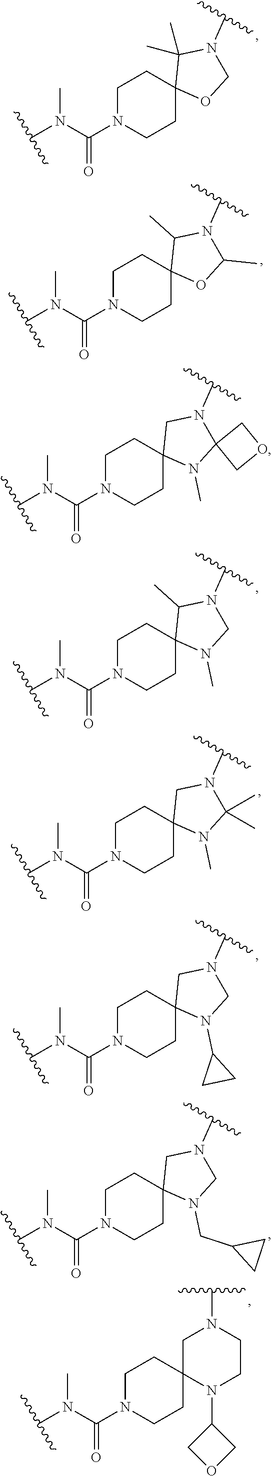

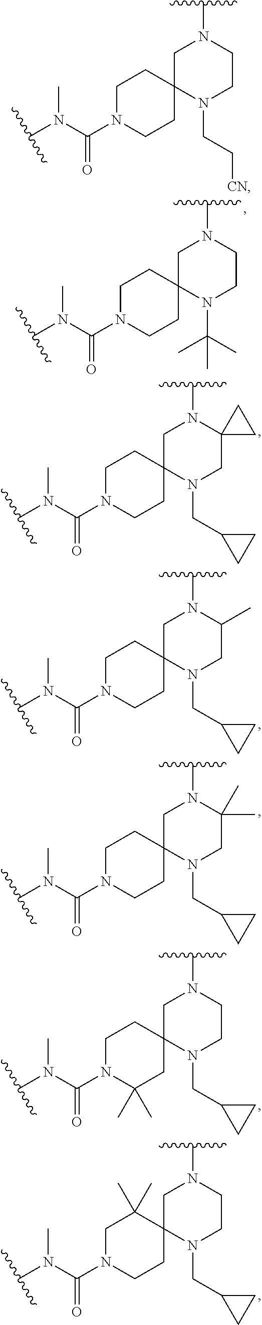

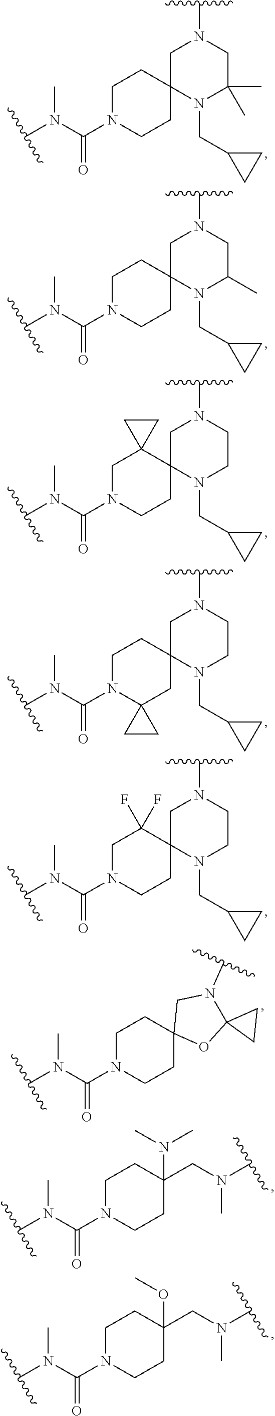

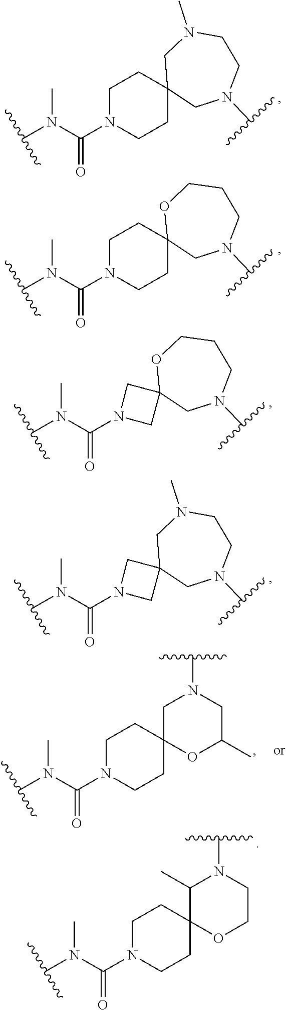 [SMILES: *N1CCN(C)CC2(CCN(C(=O)N(*)C)CC2)C1 ; *N1CCCOC2(CCN(C(=O)N(*)C)CC2)C1 ; *N1CCCOC2(C1)CN(C(=O)N(*)C)C2 ; *N1CCN(C)CC2(C1)CN(C(=O)N(*)C)C2 ; *N1CC(C)OC2(CCN(C(=O)N(*)C)CC2)C1 ; *N(C)C(=O)N1CCC2(CC1)OCCN(*)C2C]

In some embodiments, a compound of the present invention has the structure of Formula IVa9:

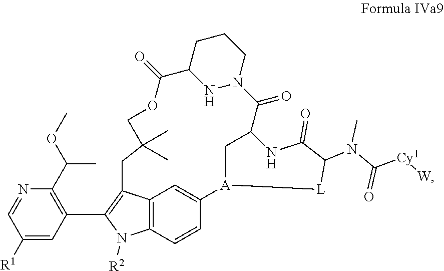

or pharmaceutically acceptable salt, an enantiomer, a stereoisomer, or a tautomer thereof, wherein Cy¹ is optionally substituted spirocyclic 8- to 11-membered heterocycloalkylene or optionally substituted bicyclic 7- to 9-membered heterocycloalkylene; and wherein W comprises a vinyl ketone or a vinyl sulfone.

In some embodiments, Cy¹ is optionally substituted spirocyclic 10- to 11-membered heterocycloalkylene.

In some embodiments, a compound of the present invention has the structure of Formula IVa10:

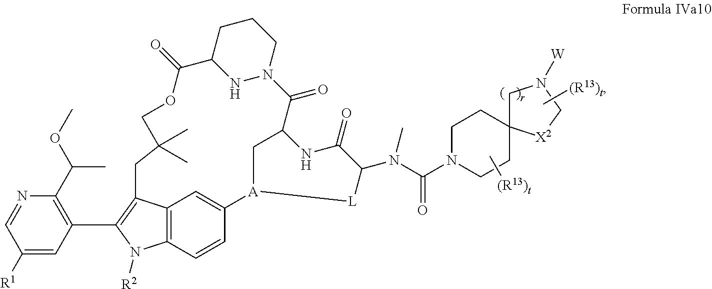

or pharmaceutically acceptable salt, an enantiomer, a stereoisomer, or a tautomer thereof, wherein X² is O, C(R¹¹)₂, NR¹², S, or SO₂;
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, or optionally substituted 3- to 5-membered cycloalkyl; and
each R¹³ is, independently, —CH₃.

In some embodiments, a compound of the present invention has the structure of Formula IVa11:

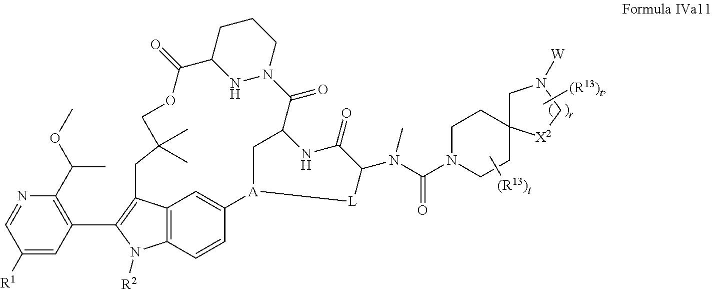

or pharmaceutically acceptable salt, an enantiomer, a stereoisomer, or a tautomer thereof, wherein X² is O, C(R¹¹)₂, NR¹², S, or SO₂;
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, optionally substituted 3- to 6-membered heterocycloalkyl, or optionally substituted 3- to 5-membered cycloalkyl; and
each R¹³ is, independently, —CH₃, F, or two R¹³ attached to the same atom combine with the atom to which they are attached to form an optionally substituted C₃-C₆ cycloalkyl, or two R¹³ attached to the same atom combine with the atom to which they are attached to form an optionally substituted 3- to 6-membered heterocycloalkyl.

In some embodiments, a compound of the present invention has the structure of Formula IVa12:

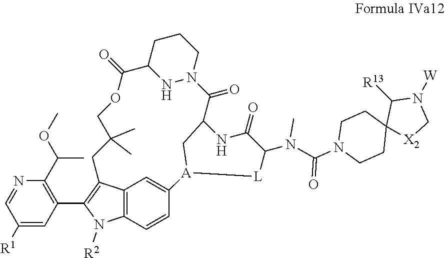

or pharmaceutically acceptable salt, an enantiomer, a stereoisomer, or a tautomer thereof.

In some embodiments, a compound of the present invention has the structure of Formula IVa13:

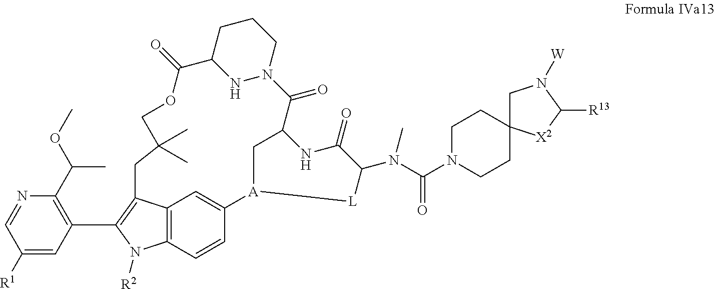

or pharmaceutically acceptable salt, an enantiomer, a stereoisomer, or a tautomer thereof.

In some embodiments, a compound of the present invention has the structure of Formula IVa14:

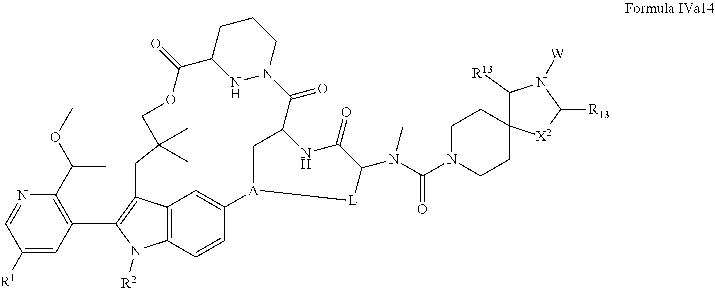

or pharmaceutically acceptable salt, an enantiomer, a stereoisomer, or a tautomer thereof.

In some embodiments, r is 1. In some embodiments, r is 2. In some embodiments, X² is 0. In some embodiments, X² is S. In some embodiments, X² is SO₂.

In some embodiments, X² is NR¹². In some embodiments, R¹² is selected from, or a stereoisomer thereof:

### —CH₃,

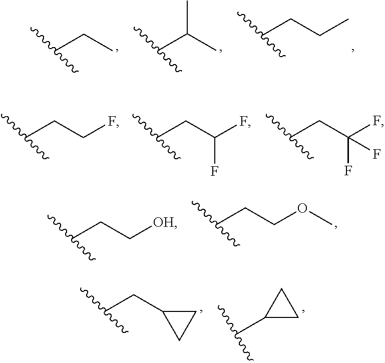

### or —H.

In some embodiments, X² is C(R¹¹)₂. In some embodiments, each R¹¹ is hydrogen.

In some embodiments, a compound of the present invention has the structure of Formula IVa15:

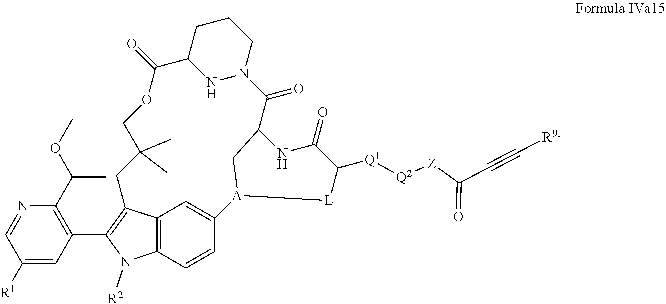

or pharmaceutically acceptable salt, an enantiomer, a stereoisomer, or a tautomer thereof, wherein Q¹ is CH₂, NR^{N}, or O;
Q² is CO, NR^{N}, or O; and
Z is optionally substituted 3- to 6-membered heterocycloalkylene or optionally substituted 5- to 10-membered heteroarylene; or
wherein Q¹-Q²-Z is an optionally substituted 9- to 10-membered spirocyclic heterocycloalkylene.

In some embodiments, a compound of the present invention has the structure of Formula IVa16:

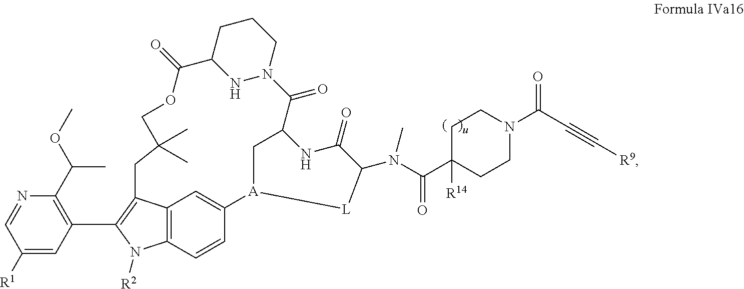

or pharmaceutically acceptable salt, an enantiomer, a stereoisomer, or a tautomer thereof, wherein R¹⁴ is fluoro, hydrogen, or C₁-C₃ alkyl; and
u is 0 or 1.

In some embodiments, R¹⁴ is fluoro and u is 1. In some embodiments, R¹⁴ is hydrogen and u is 0.

In some embodiments, a compound of the present invention has the structure of Formula IVa17:

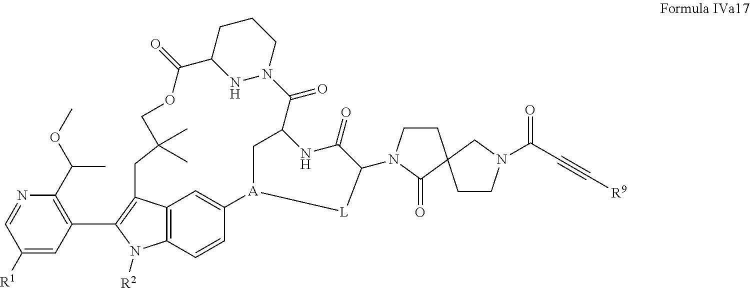

or pharmaceutically acceptable salt, an enantiomer, a stereoisomer, or a tautomer thereof.

In some embodiments, a compound of the present invention has the structure of Formula IVa18:

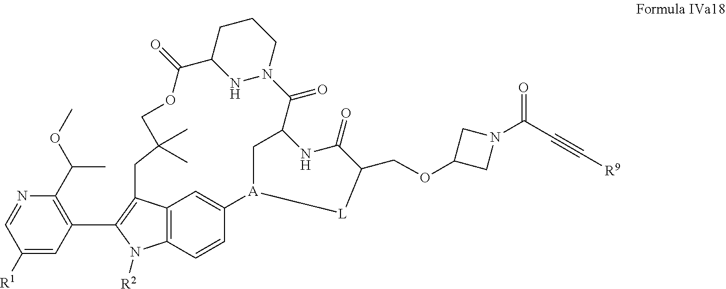

or pharmaceutically acceptable salt, an enantiomer, a stereoisomer, or a tautomer thereof.

In an aspect, the disclosure features a compound of structural Formula VIa-1:

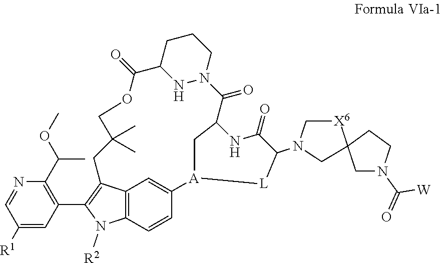

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X⁶ is CH₂ or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, or optionally substituted 3- to 10-membered heterocycloalkyl; and
R² is optionally substituted C₁-C₆ alkyl.

In an aspect, the invention features a compound having the structure of Formula Va or Formula Vb:

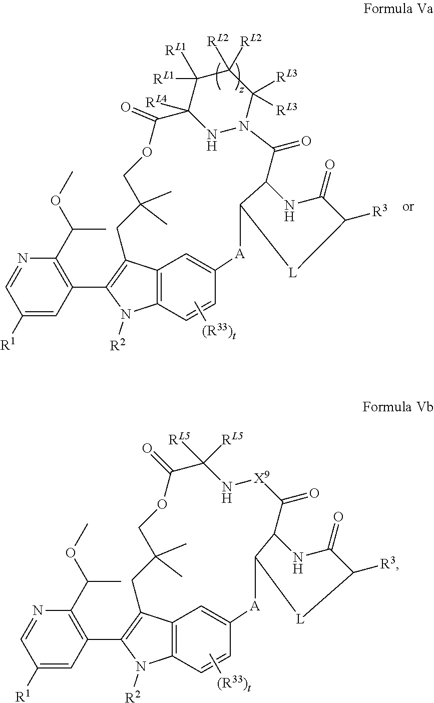

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the compound has the structure of Formula Va-1:

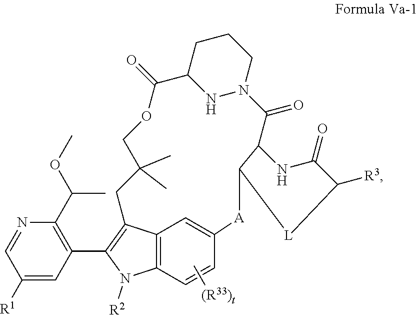

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the compound has the structure of Formula Va-2:

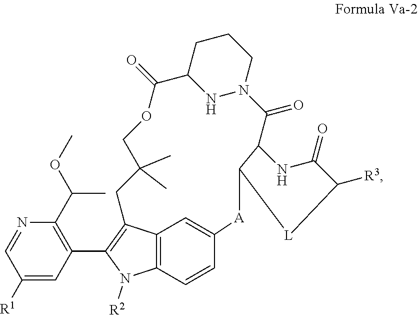

or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

In some embodiments, a compound of the present invention has the structure of Formula V-Ia:

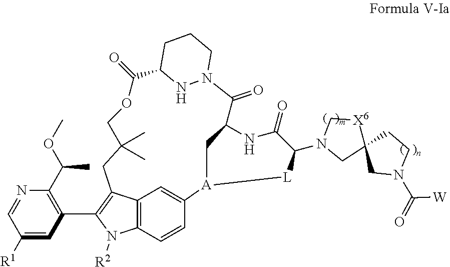

or pharmaceutically acceptable salt, an enantiomer, a stereoisomer, or a tautomer thereof.

In some embodiments, a compound of the present invention has the structure of Formula V-II-1:

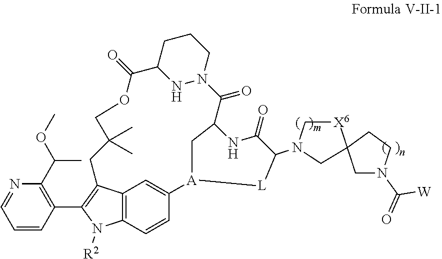

or pharmaceutically acceptable salt, an enantiomer, a stereoisomer, or a tautomer thereof.

In some embodiments, a compound of the present invention has the structure of Formula V-II-2:

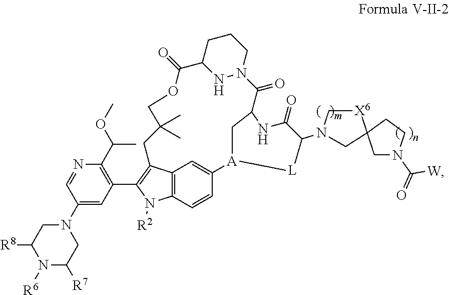

or pharmaceutically acceptable salt, an enantiomer, a stereoisomer, or a tautomer thereof, wherein R⁶, R⁷, and R⁸ are each independently selected from hydrogen, optionally substituted C₁-C₆ alkyl, optionally substituted C₁-C₆ heteroalkyl, optionally substituted 3- to 6-membered cycloalkyl, optionally substituted 3- to 6-membered heterocycloalkyl; or
R⁶ and R⁷ combine with the atoms to which they are attached to form an optionally substituted 3- to 8-membered cycloalkyl or an optionally substituted 3- to 8-membered heterocycloalkyl; or
R⁶ and R⁸ combine with the atoms to which they are attached to form an optionally substituted 3- to 8-membered cycloalkyl or an optionally substituted 3- to 8-membered heterocycloalkyl.

In some embodiments, a compound of the present invention has the structure of Formula V-II-3:

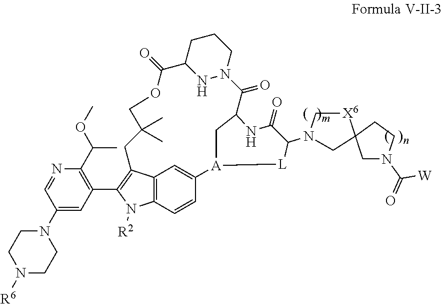

or pharmaceutically acceptable salt, an enantiomer, a stereoisomer, or a tautomer thereof.

In some embodiments, a compound of the present invention has the structure of Formula V-II-4:

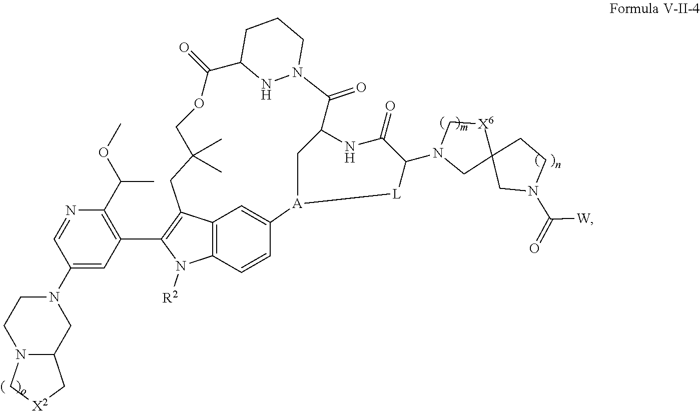

or pharmaceutically acceptable salt, an enantiomer, a stereoisomer, or a tautomer thereof, wherein X² is CH₂ or O; and
o is 1 or 2.

In some embodiments, the compound has the structure of Formula VIa or Formula VIb:

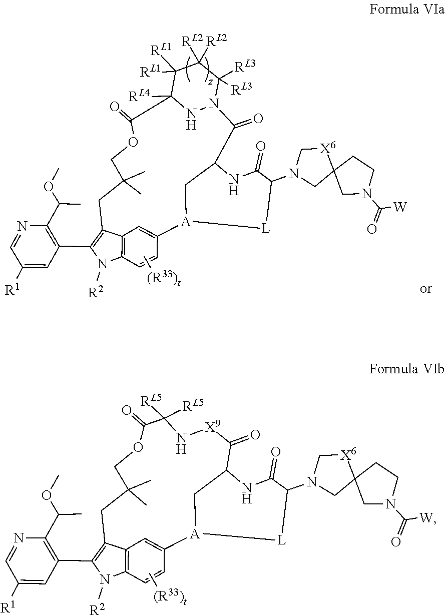

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X⁶ is CH₂ or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, or optionally substituted 3- to 10-membered heterocycloalkyl;
R² is optionally substituted C₁-C₆ alkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the compound has the structure of Formula VIa-1:

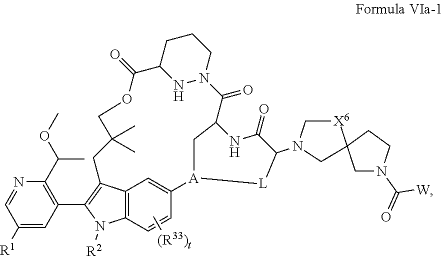

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X⁶ is CH₂ or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, or optionally substituted 3- to 10-membered heterocycloalkyl;
R² is optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the compound has the structure of Formula VIa-2:

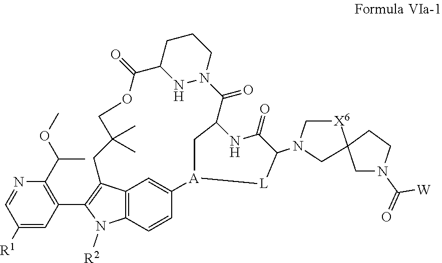

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X⁶ is CH₂ or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, or optionally substituted 3- to 10-membered heterocycloalkyl; and
R² is optionally substituted C₁-C₆ alkyl.

In some embodiments of a compound of the present invention, X² is CH₂. In some embodiments, o is 1. In some embodiments, o is 2.

In some embodiments of a compound of the present invention, X⁶ is O. In some embodiments, o is 1. In some embodiments, o is 2.

In some embodiments of a compound of the present invention, R² is:

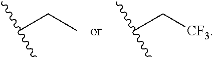

In some embodiments of a compound of the present invention, R³ is optionally substituted C₁-C₆ alkyl. In some embodiments, R³ is:

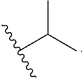 [SMILES: *C(C)C]

In some embodiments of a compound of the present invention, R³ is or optionally substituted 3- to 6-membered cycloalkyl. In some embodiments, R³ is:

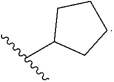 [SMILES: *C1CCCC1]

In some embodiments of a compound of the present invention, A is optionally substituted 5- to 10-membered heteroarylene. In some embodiments, A is:

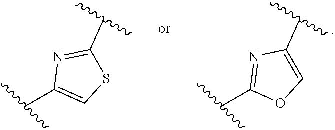 [SMILES: *c1csc(*)n1 ; *c1coc(*)n1]

In some embodiments of a compound of the present invention, A is optionally substituted phenyl. In some embodiments, A is:

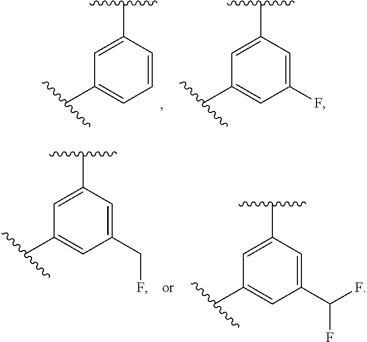 [SMILES: *c1cccc(*)c1 ; *c1cc(*)cc(F)c1 ; *c1cc(*)cc(CF)c1 ; *c1cc(*)cc(C(F)F)c1]

In some embodiments of a compound of the present invention, A is optionally substituted 3- to 6-membered heterocycloalkylene. In some embodiments, A is selected from the following, or a stereoisomer thereof:

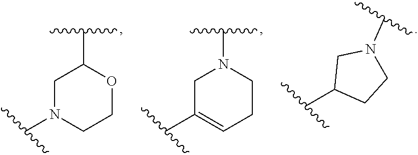 [SMILES: *C1CN(*)CCO1 ; *C1=CCCN(*)C1 ; *C1CCN(*)C1]

In some embodiments of a compound of the present invention, m is 1. In some embodiments, n is 1. In some embodiments, X¹ is CH₂. In some embodiments, X⁶ is O. In some embodiments, m is 1, n is 1, and X⁶ is CH₂. In some embodiments, m is 1, n is 1, and X⁶ is O.

In some embodiments of a compound of the present invention, m is 2. In some embodiments, X⁶ is CH₂. In some embodiments, n is 1. In some embodiments, n is 0. In some embodiments, m is 2, X⁶ is CH₂, and n is 1. In some embodiments, m is 2 and X⁶ is O. In some embodiments, m is 2, X⁶ is O, and n is 1. In some embodiments, m is 2, X⁶ is O, and n is 0.

In some embodiments of a compound of the present invention, W comprises an aziridine. In some embodiments, W comprises an optionally substituted cyclopropyl-aziridinyl moiety. In some embodiments, W is selected from the following, or a stereoisomer thereof:

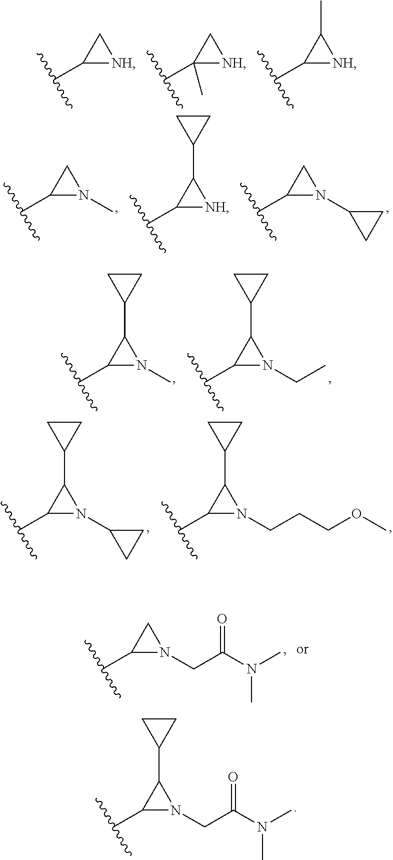

In some embodiments of a compound of the present invention, W comprises an epoxide. In some embodiments, W is selected from the following, or a stereoisomer thereof:

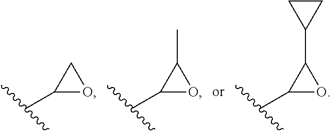

In an aspect, the invention features a compound having the structure of Formula VIIa or Formula VIIb:

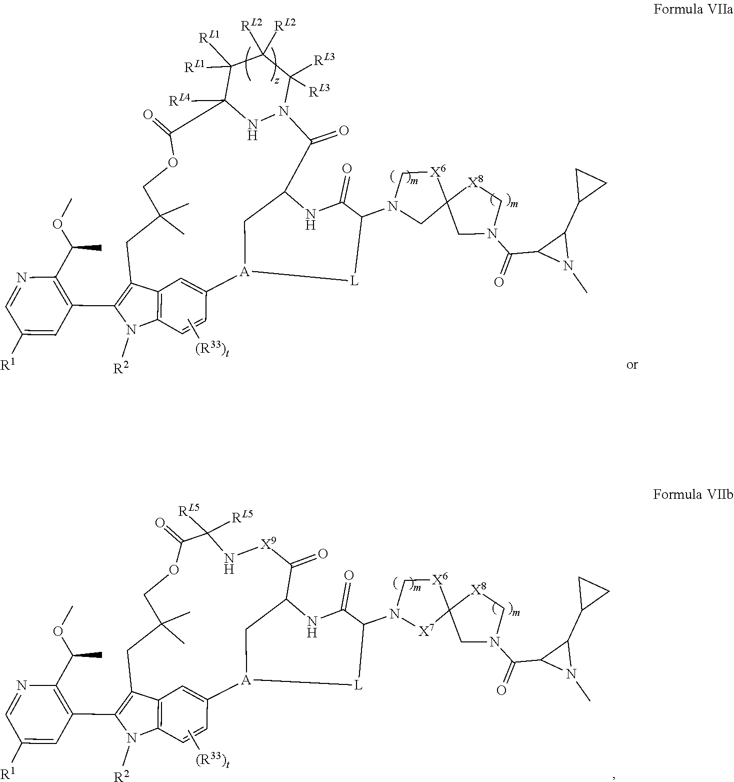

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
X⁶, X⁷, and X⁸ are each independently selected from CH₂, CHF, CF₂, C═O, or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, optionally substituted C₁-C₆ heteroalkyl, or optionally substituted 3- to 10-membered heterocycloalkyl;
R² is optionally substituted C₁-C₆ alkyl;
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the compound has the structure of Formula VIIa-1:

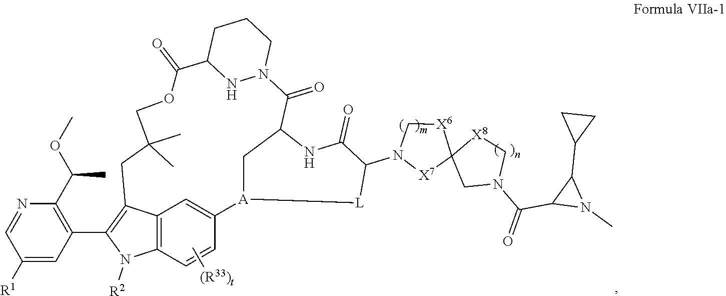

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
X⁶, X⁷, and X⁸ are each independently selected from CH₂, CHF, CF₂, C═O, or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, optionally substituted C₁-C₆ heteroalkyl, or optionally substituted 3- to 10-membered heterocycloalkyl;
R² is optionally substituted C₁-C₆ alkyl;
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, the disclosure features a compound of structural Formula VIIa-2:

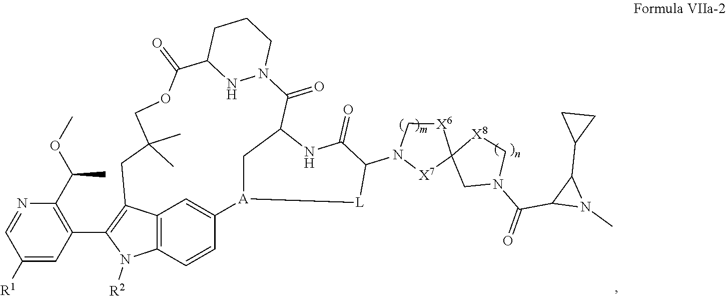

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
X⁶, X⁷, and X⁸ are each independently selected from CH₂, CHF, CF₂, C═O, or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, optionally substituted C₁-C₆ heteroalkyl, or optionally substituted 3- to 10-membered heterocycloalkyl;
R² is optionally substituted C₁-C₆ alkyl; and
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium.

In some embodiments, a compound of the present invention has the structure of Formula VI-Ia, Formula VI-Ib, Formula VI-Ic:

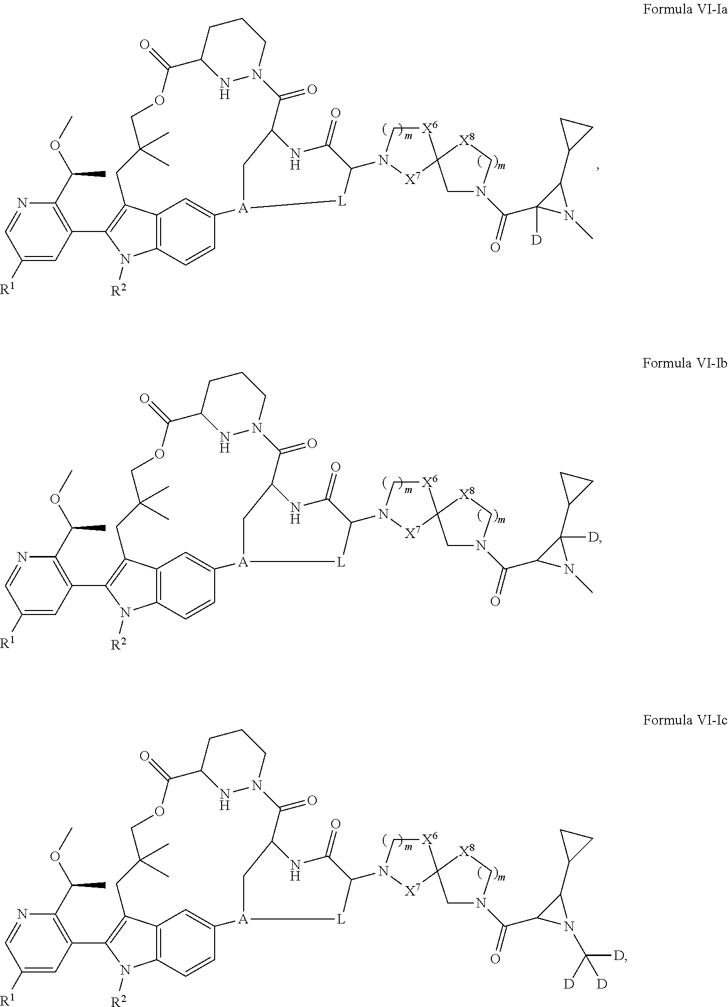

or pharmaceutically acceptable salt, an enantiomer, a stereoisomer, or a tautomer thereof, wherein each D indicates a hydrogen having an isotopic enrichment factor for deuterium of at least 5.

In some embodiments, a compound of the present invention has the structure of Formula VI-II:

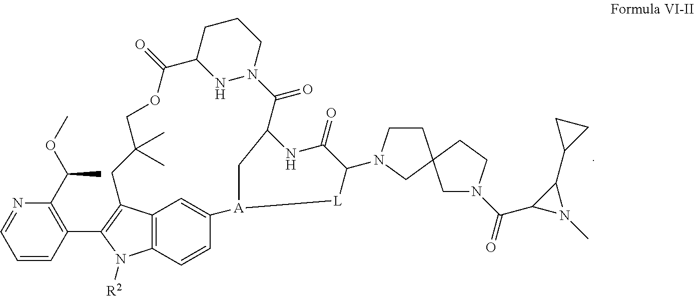

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
R² is optionally substituted C₁-C₆ alkyl; and
R³ is optionally substituted C₁-C₆ alkyl, optionally substituted 3- to 6-membered cycloalkyl, or optionally substituted heterocycloalkyl,
and wherein each hydrogen is independently, optionally, isotopically enriched for deuterium.

In some embodiments, a compound of the present invention has the structure of Formula VI-V:

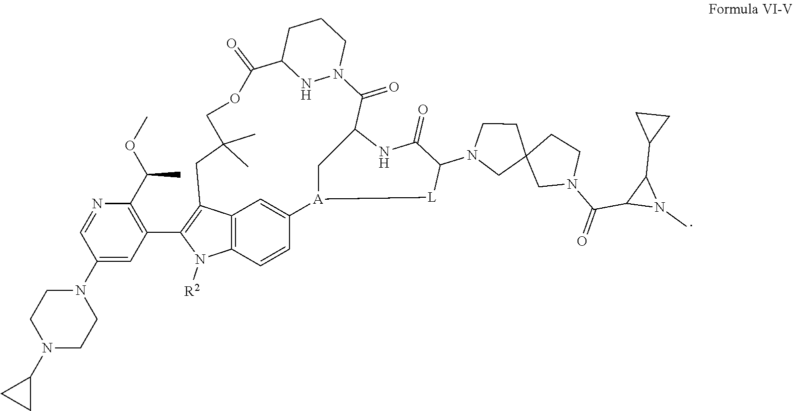

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
R² is optionally substituted C₁-C₆ alkyl; and
R³ is optionally substituted C₁-C₆ alkyl, optionally substituted 3- to 6-membered cycloalkyl, or optionally substituted heterocycloalkyl,
and wherein each hydrogen is independently, optionally, isotopically enriched for deuterium.

In some embodiments, a compound of the present invention has the structure of Formula VI-VI:

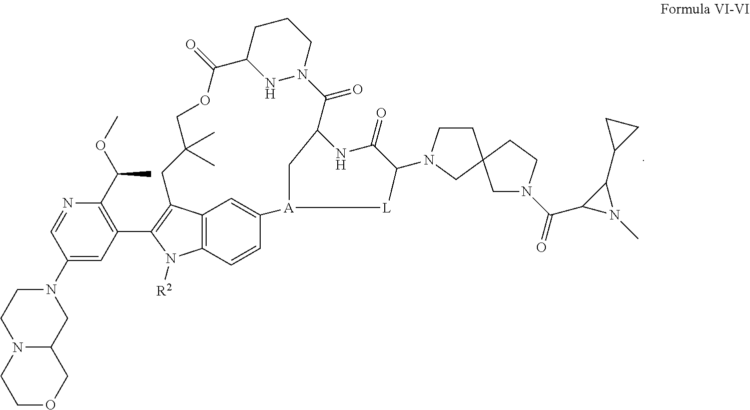

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
R² is optionally substituted C₁-C₆ alkyl; and
R³ is optionally substituted C₁-C₆ alkyl, optionally substituted 3- to 6-membered cycloalkyl, or optionally substituted heterocycloalkyl,
and wherein each hydrogen is independently, optionally, isotopically enriched for deuterium.

In some embodiments, a compound of the present invention has the structure of Formula VI-VII:

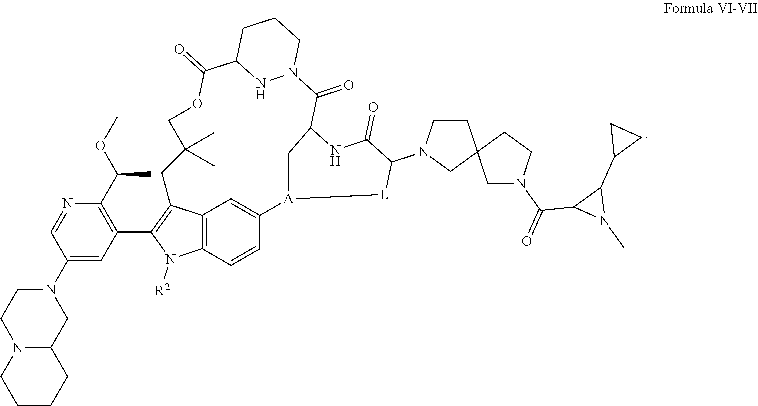

or pharmaceutically acceptable salt, an enantiomer, a stereoisomer, or a tautomer thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
R² is optionally substituted C₁-C₆ alkyl; and
R³ is optionally substituted C₁-C₆ alkyl, optionally substituted 3- to 6-membered cycloalkyl, or optionally substituted heterocycloalkyl,
and wherein each hydrogen is independently, optionally, isotopically enriched for deuterium.

In some embodiments, a compound of the present invention has the structure of Formula VI-Va, Formula VI-Vb, Formula VI-Vc:

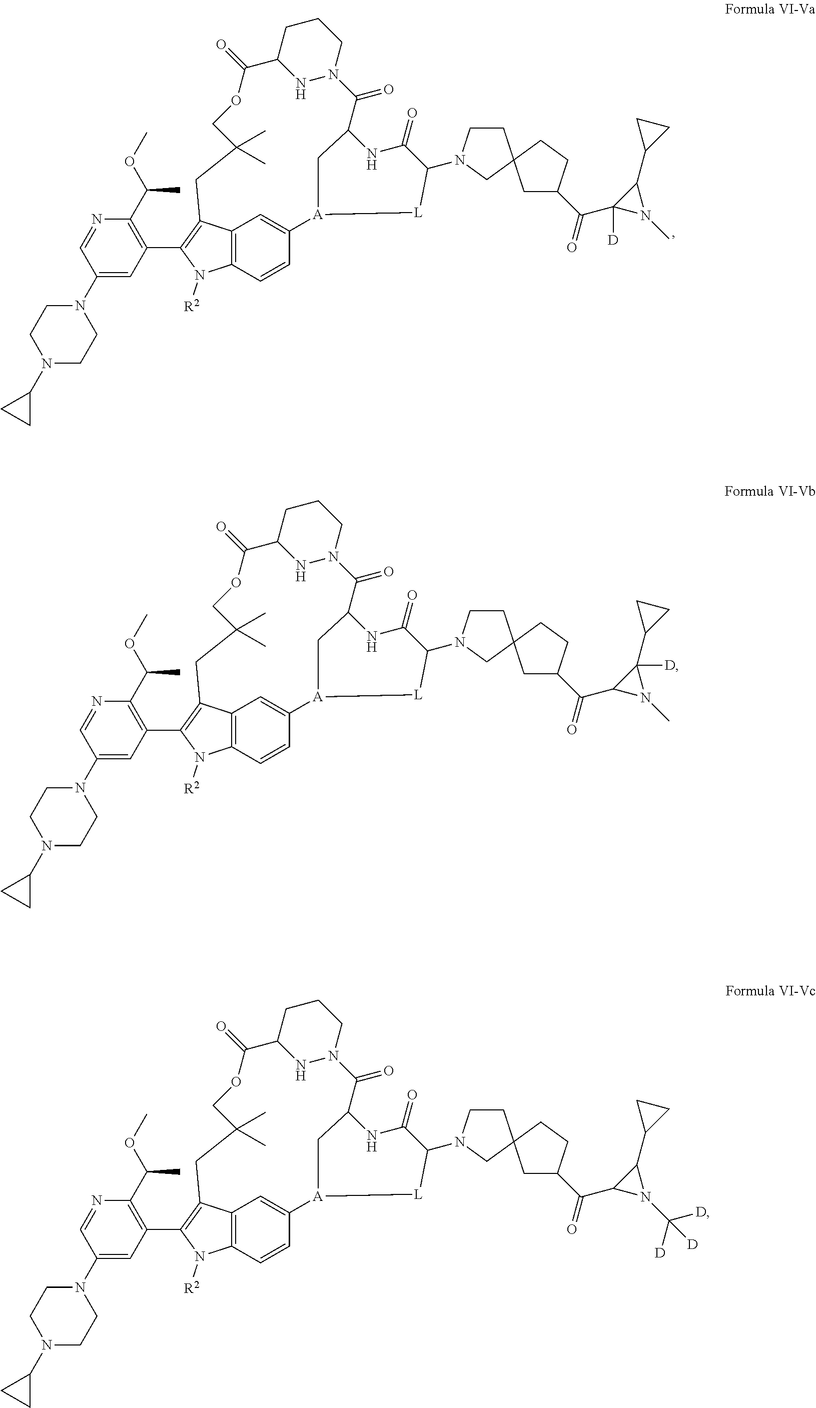

or pharmaceutically acceptable salt, an enantiomer, a stereoisomer, or a tautomer thereof, wherein each D indicates a hydrogen having an isotopic enrichment factor for deuterium of at least 5.

In some embodiments, a compound of the present invention has the structure of Formula VI-Vd, Formula VI-Ve, Formula VI-Vf:

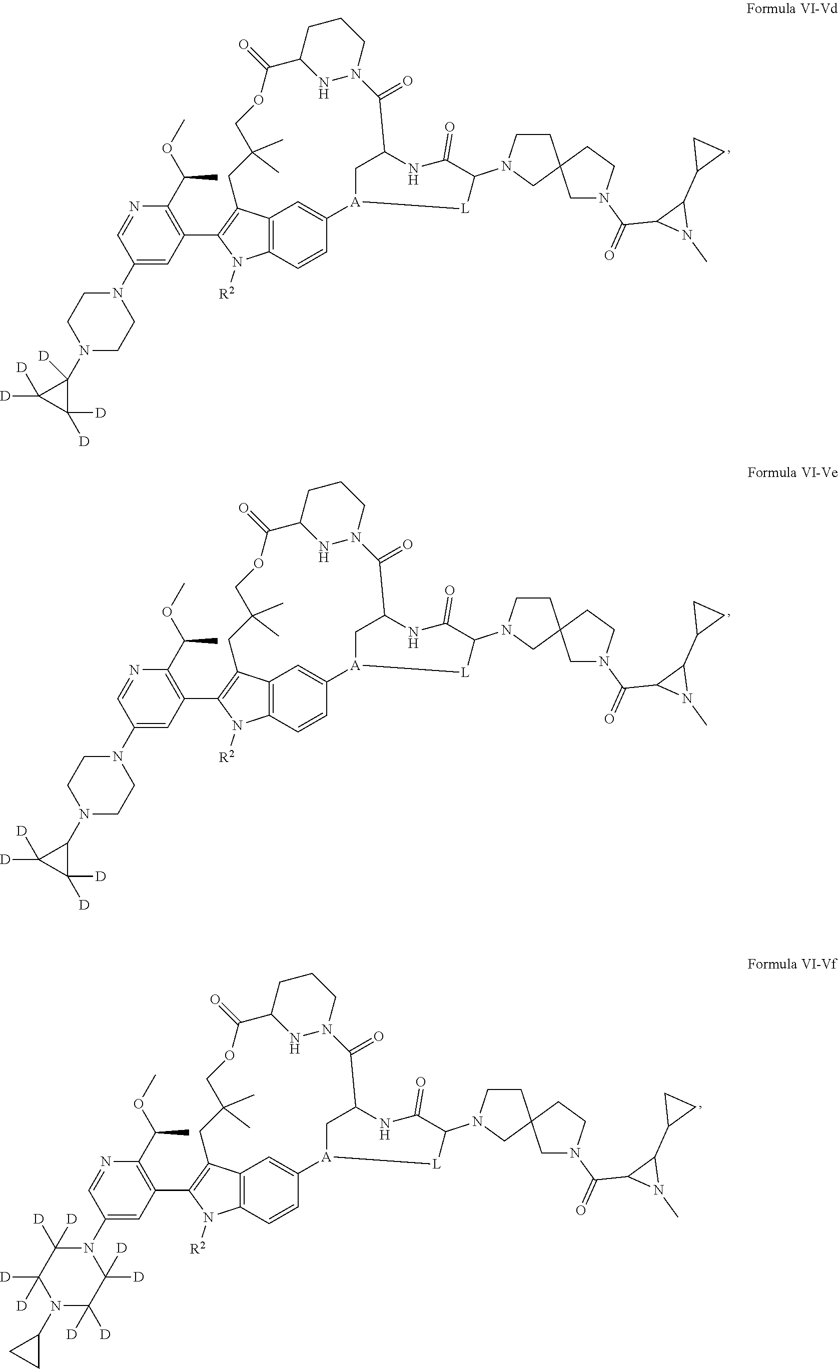

or pharmaceutically acceptable salt, an enantiomer, a stereoisomer, or a tautomer thereof, wherein each D indicates a hydrogen having an isotopic enrichment factor for deuterium of at least 5.

In an aspect, the invention features a compound having the structure of Formula XI:

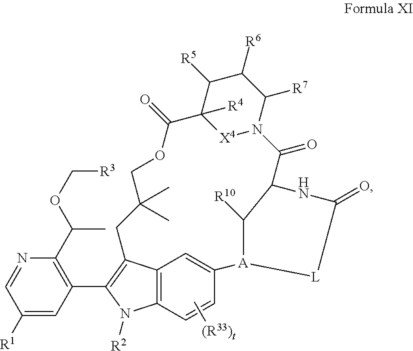

or pharmaceutically acceptable salt, an enantiomer, a stereoisomer, or a tautomer thereof, wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ alkenyl, optionally substituted C₁-C₆ alkynyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is —OR¹¹ or —NR¹²R¹³;
R¹¹, R¹², and R¹³ are each, independently, optionally substituted C₁-C₆ alkyl, optionally substituted C₁-C₆ heteroalkyl, or R¹² and R¹³ combine to form an optionally substituted 3- to 10-membered heterocycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

In some embodiments, A is optionally substituted thiazole-diyl, optionally substituted oxazole-diyl, optionally substituted morpholine-diyl, optionally substituted pyrrolidine-diyl, optionally substituted piperidine-diyl, or optionally substituted phenylene. In some embodiments, A is optionally substituted thiazole-diyl or optionally substituted morpholine-diyl. In some embodiments of a compound of the present invention, A is optionally substituted 5- to 10-membered heteroarylene. In some embodiments, A is:

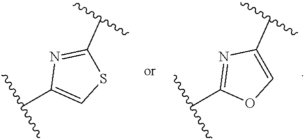 [SMILES: *c1csc(*)n1 ; *c1coc(*)n1]

In some embodiments, A is

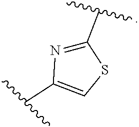 [SMILES: *c1csc(*)n1]

In some embodiments of a compound of the present invention, A is optionally substituted phenylene. In some embodiments, A is:

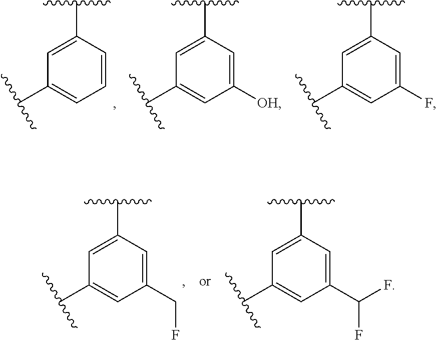

In some embodiments, A is

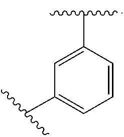 [SMILES: *c1cccc(*)c1]

In some embodiments of a compound of the present invention, A is optionally substituted 3- to 6-membered heterocycloalkylene. In some embodiments, A is optionally substituted 6-membered heterocycloalkylene. In some embodiments, A is selected from the following, or a stereoisomer thereof:

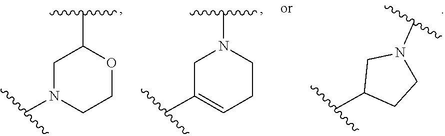 [SMILES: "or" ; *C1CN(*)CCO1 ; *C1=CCCN(*)C1 ; *C1CCN(*)C1]

In some embodiments, A is selected from the following, or a stereoisomer thereof:

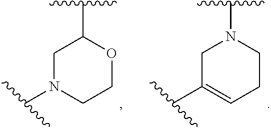 [SMILES: *C1CN(*)CCO1 ; *C1=CCCN(*)C1]

In some embodiments of a compound of the present invention, R¹ is hydrogen, optionally substituted 3- to 10-membered heterocycloalkyl, or optionally substituted C₁-C₆ heteroalkyl. In some embodiments of a compound of the present invention, R¹ is hydrogen or optionally substituted 3- to 10-membered heterocycloalkyl. In some embodiments of a compound of the present invention, R¹ is optionally substituted 3- to 10-membered heterocycloalkyl. In some embodiments of a compound of the present invention, R¹ is:

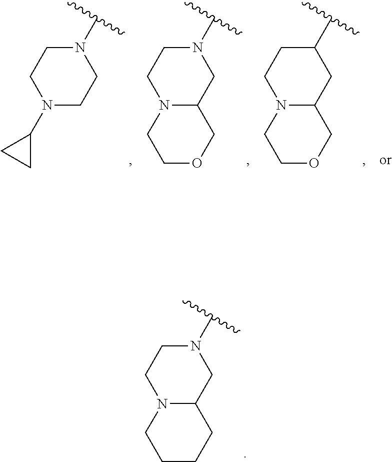 [SMILES: *N1CCN(C2CC2)CC1 ; *N1CCN2CCOCC2C1 ; *C1CCN2CCOCC2C1 ; *N1CCN2CCCCC2C1]

In some embodiments of a compound of the present invention, R¹ is:

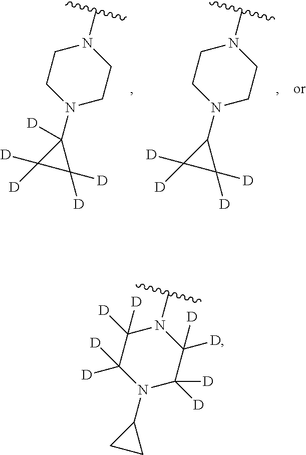

wherein each D indicates a hydrogen having an isotopic enrichment factor for deuterium of at least 5.

In some embodiments of a compound of the present invention, R² is:

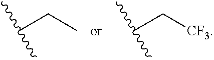

In some embodiments of a compound of the present invention, R² is:

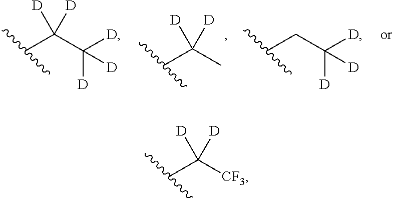

and wherein each D indicates a hydrogen having an isotopic enrichment factor for deuterium of at least 5.

In some embodiments of a compound of the present invention, R³ is optionally substituted C₁-C₆ alkyl or optionally substituted 3- to 6-membered cycloalkyl. In some embodiments of a compound of the present invention, R³ is optionally substituted C₁-C₆ alkyl. In some embodiments, R³ is:

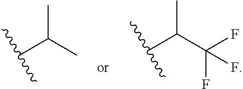

In some embodiments, R³ is:

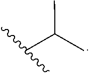 [SMILES: *C(C)C]

In some embodiments, R³ is:

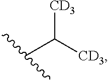 [SMILES: *C(C([2H])([2H])[2H])C([2H])([2H])[2H]]

and wherein each D indicates a hydrogen having an isotopic enrichment factor for deuterium of at least 5.

In some embodiments of a compound of the present invention, R³ is or optionally substituted 3- to 6-membered cycloalkyl. In some embodiments, R³ is:

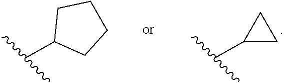

In some embodiments, R³ is:

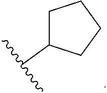 [SMILES: *C1CCCC1]

In some embodiments of a compound of the present invention, R² is

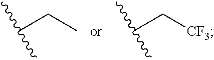

### R³ is

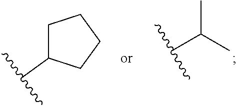

### and A is

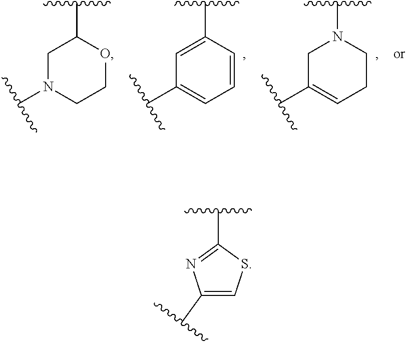

In some embodiments, R² is

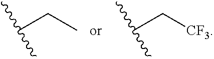

### R³ is

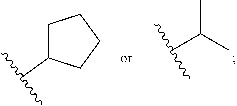

### and A is

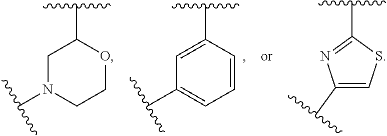

In some embodiments of a compound of the present invention, m is 1. In some embodiments, n is 1. In some embodiments, X¹ is CH₂. In some embodiments, X² is CH₂. In some embodiments, X³ is CH₂. In some embodiments, m is 1, n is 1, and each of X¹, X², and X³ is CH₂.

In an aspect, the invention features a compound having the structure of Formula Ic:

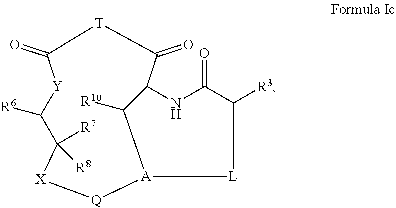

or pharmaceutically acceptable salt, an enantiomer, a stereoisomer, or a tautomer thereof, wherein:
Q is an optionally substituted 7- to 12-membered bicyclic arylene, an optionally substituted 7- to 12-membered bicyclic heteroarylene, an optionally substituted 7- to 12-membered bicyclic heterocyclylene, wherein a first ring in Q is bonded to X, and a second ring in Q is bonded to A;
X is a bond; a straight chain C₁-C₃ alkylene optionally substituted with 1 to 3 substituents independently selected from fluoro, —CN, —C₁-C₃ alkyl, and —O—C₁-C₃ alkyl; —O—; —S(O)₀₋₂—; *—CH₂—O—; *—CH₂—S(O)₀₋₂—; *—O—CH₂—; or *—CH₂—S(O)₀₋₂—, wherein “*” represents a portion of X bound to —C(R⁷)(R⁸)—;
Y is —O—, —NH— or —N(C₁-C₃ alkyl)-;
A is optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
L is a linker;
T is a second linker;
R³ is optionally substituted C₁-C₆ alkyl, optionally substituted C₃-C₆ cycloalkyl, optionally substituted C₆ aryl, or optionally substituted 3- to 7-membered heterocyclyl;
R¹⁰ is hydrogen, halogen, optionally substituted C₁-C₃ alkyl, or C₁-C₃ optionally substituted heteroalkyl;
R⁷ is hydrogen, halogen, or optionally substituted C₁-C₃ alkyl;
R⁸ is hydrogen, halogen, —OH, —CN, —O-(optionally substituted C₁-C₃ alkyl), optionally substituted C₁-C₃ alkyl, optionally substituted C₂-C₆ alkenyl, optionally substituted C₂-C₆ alkynyl, optionally substituted C₆-C₁₀ aryl, optionally substituted 4- to 8-membered heteroaryl, optionally substituted C₃-C₆ cycloalkyl, or optionally substituted 3- to 7-membered heterocyclyl; or
R⁷ and R⁸ together form ═CH₂, an optionally substituted C₃-C₆ cycloalkyl, or a 3- to 7-membered saturated heterocyclyl; or
R⁸ and a ring atom in Q, the carbon atom to which R⁷ is bound, and X to form a 4- to 9-membered saturated or unsaturated heterocyclyl that is fused to Q;
R⁶ is hydrogen or —CH₃;
each R⁵ is independently halogen, optionally substituted C₁-C₃ alkyl, or optionally substituted
C₁-C₃ haloalkyl;
p is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In some embodiments of any of the compounds described herein, T has the structure of Formula XV:

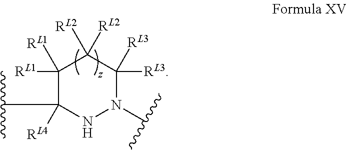

In some embodiments of Formula XV, z is 0.

In some embodiments of any of the compounds described herein, T has the structure of Formula XVa:

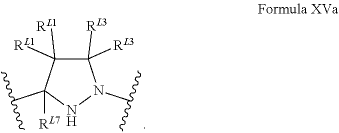

In some embodiments, T has the structure of

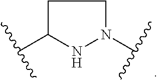 [SMILES: *C1CCN(*)N1]

In some embodiments, of Formula XV, z is 1.

In some embodiments of any of the compounds described herein, T has the structure of Formula XVb:

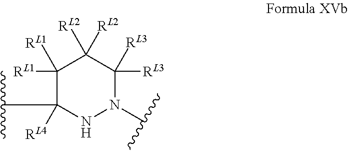

In some embodiments of any of the compounds described herein, T has the structure of Formula XVc:

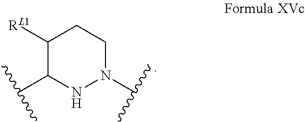

In some embodiments of any of the compounds described herein, T has the structure of Formula XVd:

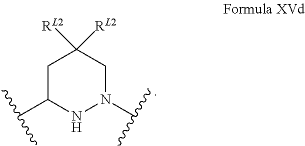

In some embodiments of any of the compounds described herein, T has the structure of Formula XVe:

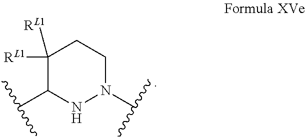

In some embodiments of Formula XV, z is 2.

In some embodiments of any of the compounds described herein, T has the structure of Formula XVf:

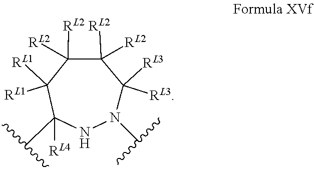

In some embodiments, wherein R^{L1} is hydrogen. In some embodiments, R^{L1} is optionally substituted C₁-C₆ alkyl. In some embodiments, R^{L1} is methyl, ethyl, or trifluoromethyl. In some embodiments, R^{L1} is optionally substituted C₁-C₆ heteroalkyl. In some embodiments, R^{L1} is methoxy or ethoxy. In some embodiments, R^{L1} is optionally substituted C₂-C₆ alkynyl. In some embodiments, R^{L1} is ethynyl.

In some embodiments, R^{L2} is hydrogen. In some embodiments, R^{L2} is halogen. In some embodiments, R^{L2} is fluoro.

In some embodiments, R^{L3} is hydrogen. In some embodiments, R^{L3} is optionally substituted C₁-C₆ alkyl. In some embodiments, R^{L3} is methyl.

In some embodiments, R^{L4} is hydrogen.

In some embodiments, R^{L1} and R^{L4} combine to form an optionally substituted C₄ cycloalkyl.

In some embodiments, R^{L1} and R^{L3} combine to form an optionally substituted C₄ cycloalkyl. In some embodiments, R^{L1} and R^{L3} combine to form an optionally substituted C₅ cycloalkyl.

In some embodiments, two R^{L1} combine to form an optionally substituted C₃-C₆ cycloalkyl.

In some embodiments, R^{L1} and R^{L2} combine to form an optionally substituted C₃-C₆ cycloalkyl.

In some embodiments, T is:

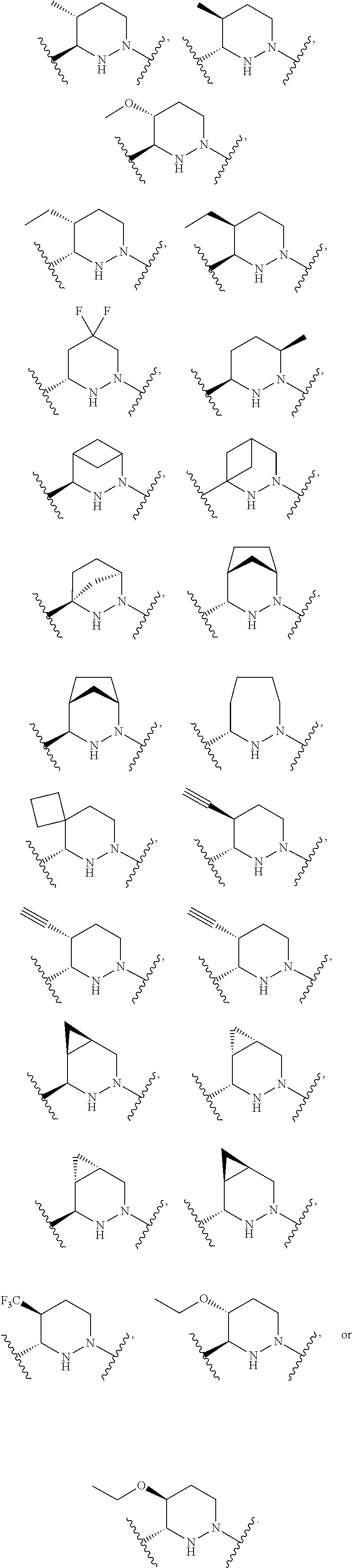

In some embodiments, T is:

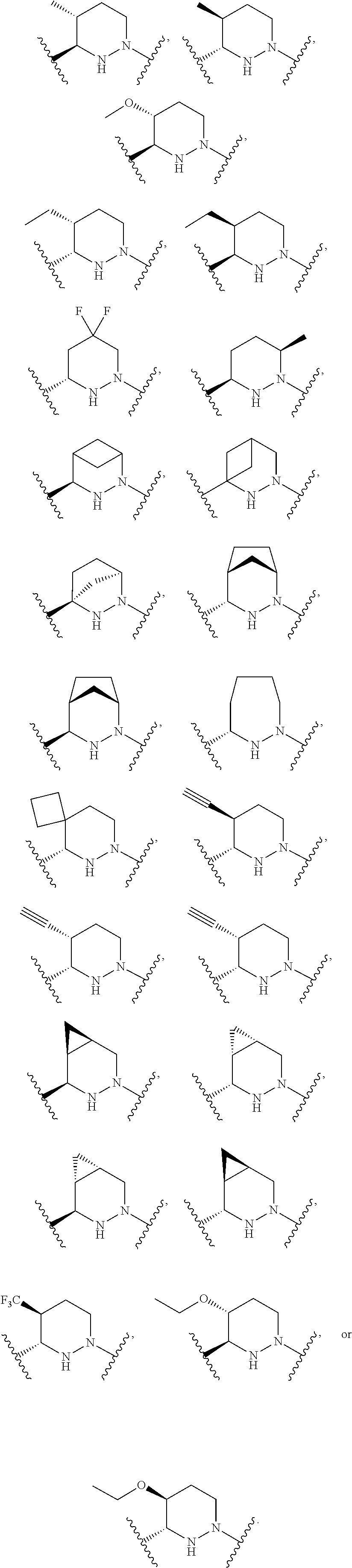

In some embodiments of any of the compounds described herein, T has the structure of Formula XVI:

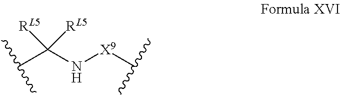

In some embodiments, X⁹ is —NR^{L6}—.

In some embodiments of any of the compounds described herein, T has the structure of Formula XVIa:

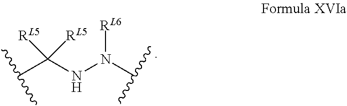

In some embodiments of any of the compounds described herein, T has the structure of Formula XVIb:

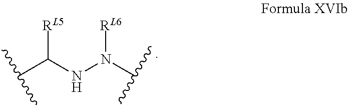

In some embodiments, R^{L6} is optionally substituted C₁-C₆ alkyl. In some embodiments, R^{L6} is methyl.

In some embodiments, X⁹ is —C(O)—. In some embodiments, X⁹ is —S(O)₂—.

In some embodiments, R^{L5} is hydrogen. In some embodiments, R^{L5} is optionally substituted C₁-C₆ alkyl. In some embodiments, R^{L5} is optionally substituted C₃-C₈ cycloalkyl. In some embodiments, two R^{L5} combine to form an optionally substituted C₃-C₈ cycloalkyl.

In some embodiments of any of the compounds described herein, T is:

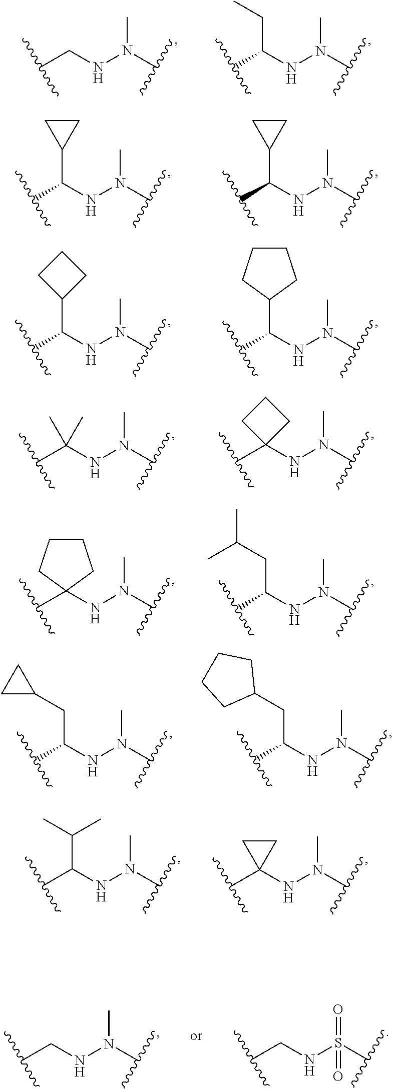

In some embodiments of any of the compounds described herein, T is:

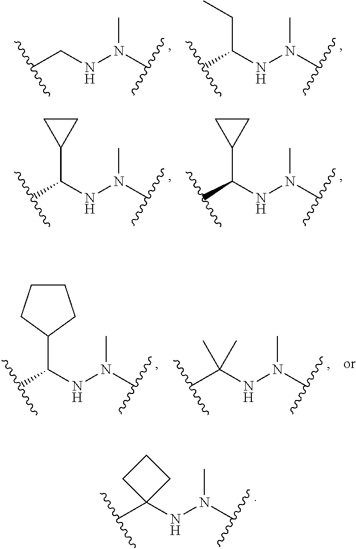

In some embodiments of any of the compounds described herein, T does not have the structure of:

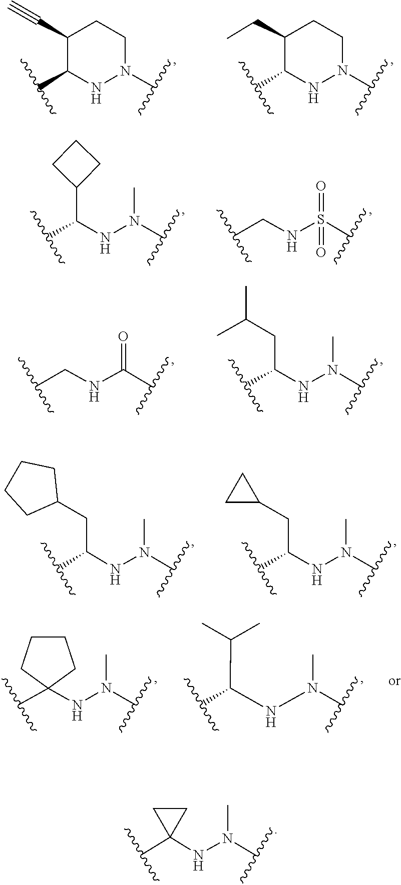

In some embodiments, L has the structure of Formula XIII:

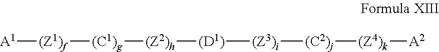

wherein A¹ is a bond between the linker and the rest of the macrocycle; A² is a bond between A and the linker; Z¹, Z², Z³, and Z⁴ are each, independently, optionally substituted C₁-C₃ alkylene, optionally substituted C₁-C₃ heteroalkylene, optionally substituted C₁-C₂ alkenylene, optionally substituted 3- to 8-membered heterocycloalkylene, optionally substituted 3- to 8-membered cycloalkylene, O, NR^{N} or a cross-linking group comprising a vinyl ketone, an ynone, a vinyl sulfone, an alkynyl sulfone, a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal; R^{N} is hydrogen, optionally substituted C₁-C₄ alkyl, or optionally substituted 6-membered arylene; C¹ and C² are each, independently, carbonyl or O; f, g, h, i, j, and k are each, independently, 0 or 1; and D¹ is optionally substituted C₁-C₂ alkylene, optionally substituted C₂-C₆ alkenylene, optionally substituted C₂-C₆ alkynylene, optionally substituted 3- to 8-membered heterocycloalkylene, optionally substituted 3- to 8-membered cycloalkylene, or optionally substituted C₁-C₃ heteroalkylene, optionally substituted 6-membered arylene, or optionally substituted 5- to 10-membered heteroarylene, or a chemical bond linking A¹-(Z¹)_{f}—(C¹)_{g}—(Z²)ₕ— to —(Z³)ᵢ—(C²)ⱼ—(Z⁴)ₖ-A².

In some embodiments of linkers of Formula XIII, f is 0. In some embodiments, f is 1. In some embodiments, g is 0. In some embodiments, g is 1. In some embodiments, h is 0. In some embodiments, h is 1. In some embodiments, i is 0. In some embodiments, j is 1. In some embodiments, k is 0. In some embodiments, k is 1.

In some embodiments of linkers of Formula XIII, Z¹ is NR^{N}. In some embodiments, R^{N} is optionally substituted C₁-C₄ alkyl. In some embodiments, R^{N} is methyl.

In some embodiments of linkers of Formula XIII, C¹ is carbonyl.

In some embodiments of linkers of Formula XIII, D¹ is 3- to 8-membered cycloalkylene. In some embodiments, D¹ is optionally substituted C₁-C₂ alkylene, optionally substituted C₂-C₆ alkenylene, optionally substituted C₂-C₆ alkynylene, or optionally substituted C₁-C₃ heteroalkylene. In some embodiments, D¹ is optionally substituted 3- to 8-membered heterocycloalkylene.

In some embodiments of linkers of Formula XIII, Z⁴ is O. In some embodiments, Z⁴ is optionally substituted C₁-C₃ alkylene. In some embodiments, Z³ is optionally substituted C₁-C₃ alkylene.

In some embodiments, L has the structure of Formula VIII:

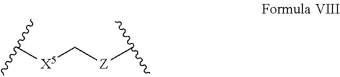

wherein X⁵ is O or CH₂ and is attached to ring A; and
Z is optionally substituted 3- to 6-membered heterocycloalkylene, optionally substituted C₁-C₆ alkylene, or optionally substituted C₁-C₆ heteroalkylene.

In some embodiments of linkers of Formula VIII, X⁵ is O.

In some embodiments of linkers of Formula VIII, Z is optionally substituted 3- to 6-membered heterocycloalkylene. In some embodiments, Z is optionally substituted 5-membered heterocycloalkylene. In some embodiments, Z is optionally substituted pyrollidine-diyl.

In some embodiments, L has the structure of Formula VIIIa:

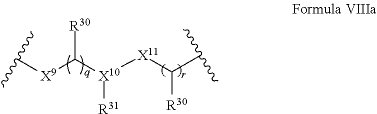

wherein X⁹ is NR, O, or CH₂ and is attached to ring A;
X¹⁰ is CH or N;
X¹¹ is NR″, O, C(O), C(O)N(R″′)₂, or CH₂;
R″ is hydrogen, cyano, optionally substituted C₁-C₄ alkyl, optionally substituted C₂-C₄ alkenyl, optionally substituted C₂-C₄ alkynyl, C(O)R″′, C(O)OR″′, C(O)N(R″′)₂, S(O)R″′, S(O)₂R″′, or S(O)₂N(R″′)₂;
each R″′ is, independently, hydrogen, optionally substituted C₁-C₄ alkyl, or optionally substituted 3- to 6-membered heterocycloalkylene;
R³⁰ and R³² are, independently, hydrogen, optionally substituted C₆-C₁₀ aryl, or optionally substituted C₁-C₆ alkylene;
R³¹ is hydrogen, optionally substituted C₆-C₁₀ aryl, optionally substituted 4- to 8-membered heteroaryl, optionally substituted 3- to 6-membered cycloalkylene, optionally substituted 3- to 6-membered heterocycloalkylene, optionally substituted C₁-C₆ alkylene, or optionally substituted C₁-C₆ heteroalkylene; and
q and r are, independently, 0, 1, 2, or 3.

In some embodiments, L has the structure of Formula VIIIb:

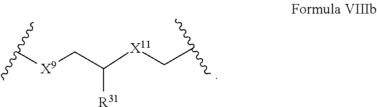

In some embodiments, L has the structure of Formula VIIIc:

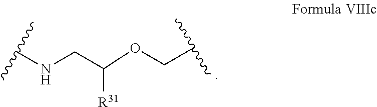

In some embodiments, L has the structure of Formula VIIId:

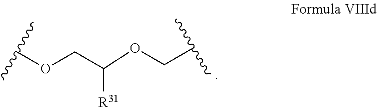

In some embodiments, L has the structure of Formula VIIIe:

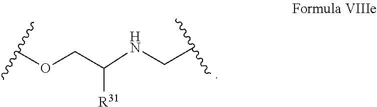

In some embodiments, L has the structure of Formula VIIIf:

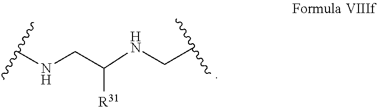

In some embodiments, L has the structure of Formula VIIIg:

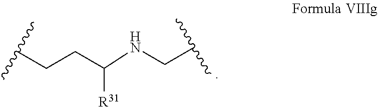

In some embodiments of linkers of Formula VIIIa, the linker is:

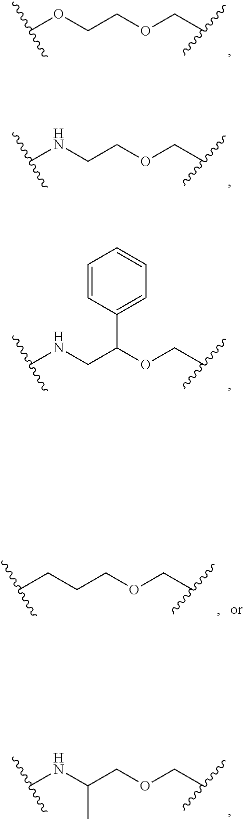

In some embodiments, the linker has the following structure:

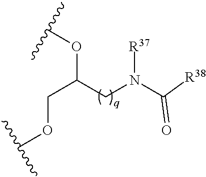

wherein R³⁷ is hydrogen or substituted C₁-C₄ alkyl;
R³⁸ is hydrogen, optionally substituted C₁-C₄ alkyl, optionally substituted 3- to 7-membered heterocycloalkyl, optionally substituted C₁-C₆ heteroalkyl, optionally substituted 3- to 6-membered cycloalkyl, or optionally substituted spirocyclic 8- to 11-membered heterocycloalkylene; and
q is, 0, 1, 2, or 3.

In some embodiments, the linker has the following structure:

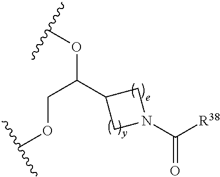

wherein R³⁸ is hydrogen, optionally substituted C₁-C₄ alkyl, optionally substituted 3- to 7-membered heterocycloalkyl, optionally substituted C₁-C₆ heteroalkyl, optionally substituted 3- to 6-membered cycloalkyl, or optionally substituted spirocyclic 8- to 11-membered heterocycloalkylene; and
y and e are, independently, 1, 2, or 3.

In some embodiments, the linker has a structure of Formula XII:

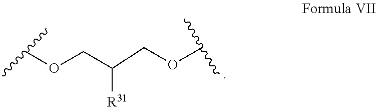

wherein the left O atom is attached to ring A;
R³⁵ is NR³⁶C(O)CH₂N(R³⁶)₂ or optionally substituted 3- to 6-membered heterocycloalkylene; and
each R³⁶ is optionally substituted C₁-C₄ alkyl.

In some embodiments, the linker has a structure of Formula XIV:

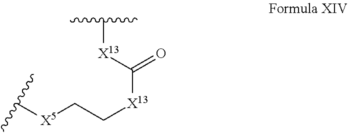

wherein
X⁵ is O and is attached to ring A;
each X¹³ is, independently, O or NR³⁴; and
each R³⁴ is, independently, hydrogen or optionally substituted C₁-C₆ alkyl.

In some embodiments, L has the structure of Formula IX:

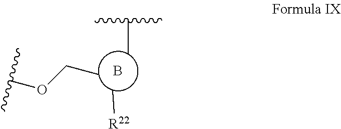

wherein
B is an optionally substituted 3- to 6-membered heterocycloalkylene;
R²² is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

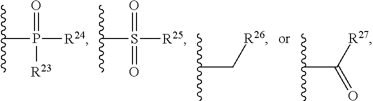

R²³ and R²⁴ are each, independently, hydrogen or optionally substituted C₁-C₆ alkyl;
R²⁵ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R²⁶ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R²⁷ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂— C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

In some embodiments, the linker of Formula IX has the structure of Formula X:

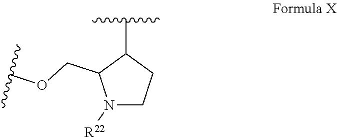

In some embodiments of linkers of Formula IX or X, R²² is

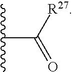

In some embodiments, R²⁷ is optionally substituted C₁-C₆ alkyl. In some embodiments, R²⁷ is optionally substituted C₂-C₆ alkenyl. In some embodiments, R²⁷ is optionally substituted C₂-C₆ alkynyl. In some embodiments, R²⁷ is optionally substituted C₁-C₆ heteroalkyl. In some embodiments, R²⁷ is optionally substituted C₂-C₆ heteroalkenyl. In some embodiments, R²⁷ is optionally substituted C₂-C₆ heteroalkynyl. In some embodiments, R²⁷ is optionally substituted C₃-C₁₀ cycloalkenyl. In some embodiments, R²⁷ is hydrogen. In some embodiments, R²⁷ is optionally substituted C₃-C₁₀ cycloalkyl. In some embodiments, R²⁷ is optionally substituted 3- to 10-membered heterocyclyl.

In some embodiments of linkers of Formula IX or X, R²² is

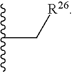

In some embodiments, R²⁶ is optionally substituted 5- to 10-membered heteroaryl. In some embodiments, R²⁶ is optionally substituted 3- to 10-membered heterocyclyl.

In some embodiments of linkers of Formula IX or X, R²² is optionally substituted 3- to 6-membered heterocyclyl.

In some embodiments of compounds of Formula I, Formula II, Formula III, Formula IV, Formula IVa, Formula IVb, Formula IVc, Formula V, or Formula VI, A is optionally substituted 3- to 6-membered heterocycloalkylene, optionally substituted 6-membered arylene, or optionally substituted 5- to 10-membered heteroarylene. In some embodiments, A is optionally substituted 6-membered arylene. In some embodiments, A is:

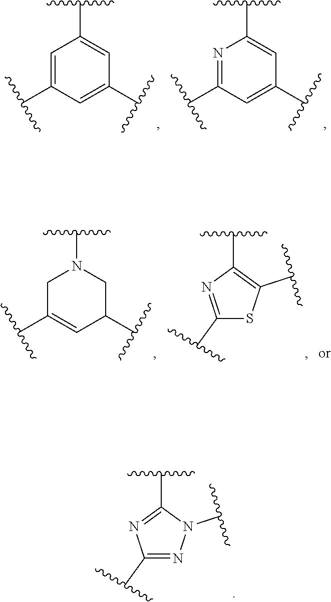 [SMILES: *c1cc(*)cc(*)c1 ; *c1cc(*)nc(*)c1 ; *C1=CC(*)CN(*)C1 ; *c1nc(*)c(*)s1 ; *c1nc(*)n(*)n1]

In some embodiments, A is:

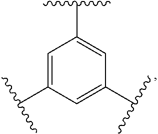 [SMILES: *c1cc(*)cc(*)c1]

In any embodiment herein, a compound of the present invention may be modified with a substituent as found in any one or more of the following applications, incorporated herein by reference in their entireties: WO 2024/060966, WO 2024/017859, WO 2024/008834, WO 2024/008610, WO 2023/232776, WO 2023/208005, WO 2023/086341, WO 2023/025832, WO 2023/015559, CN 117720556, CN 117720555, CN 117720554, CN 117534687, CN 117534685 and CN 117534684.

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex# | Structure |
|---|---|
| A1 | 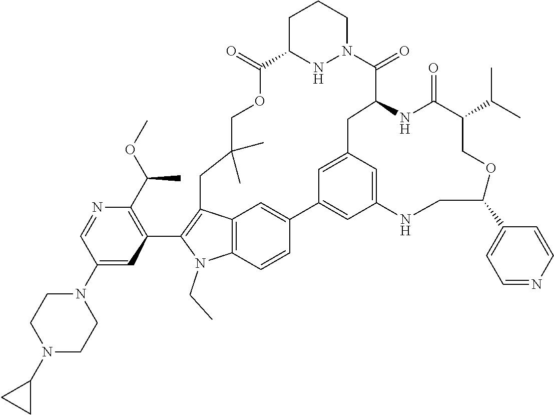 |
| A2 | 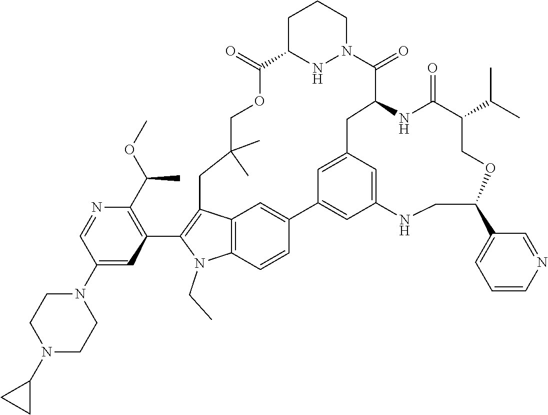 |
| A3 | 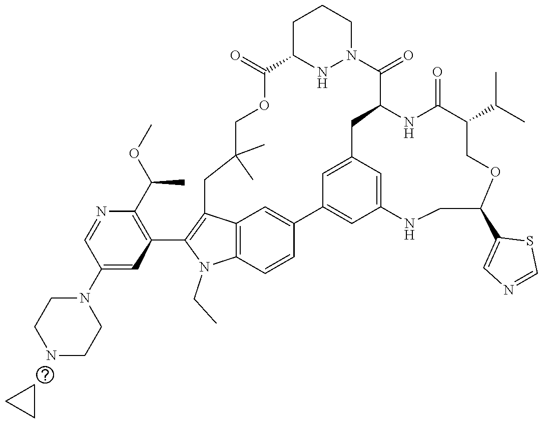 |
| A7 | 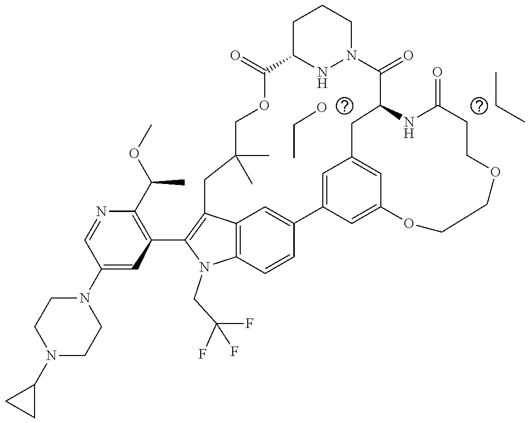 |
| A8 | 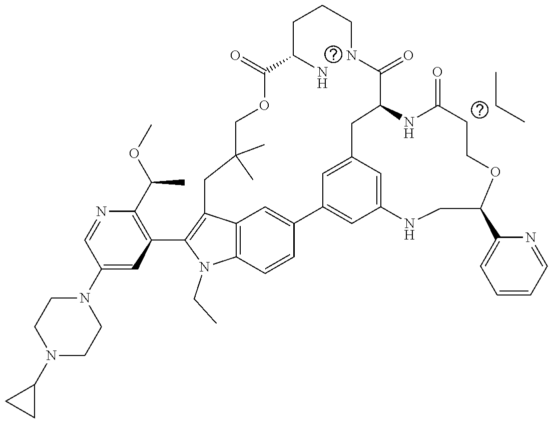 |
| A9 | 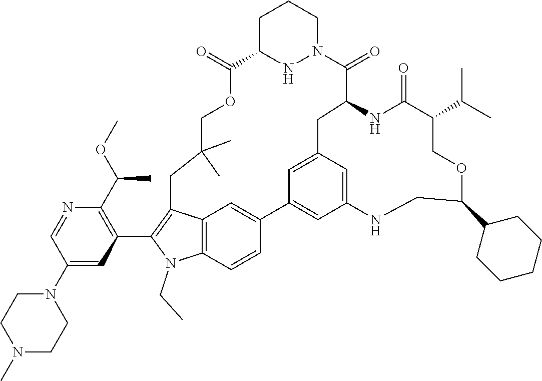 |
| A10 | 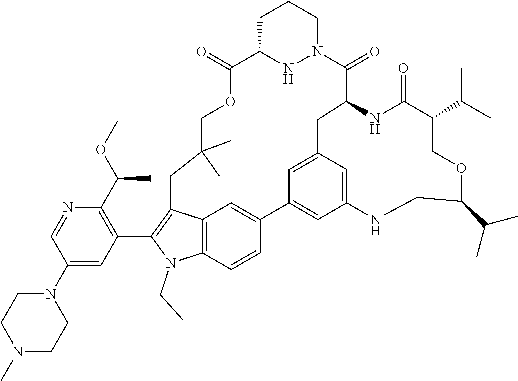 [SMILES: CCn1c(-c2cc(N3CCN(C)CC3)cnc2[C@H](C)OC)c2c3cc(ccc31)-c1cc3cc(c1)NC[C@H](C(C)C)OC[C@@H](C(C)C)C(=O)N[C@@H](C3)C(=O)N1CCC[C@H](N1)C(=O)OCC(C)(C)C2] |
| A11 | 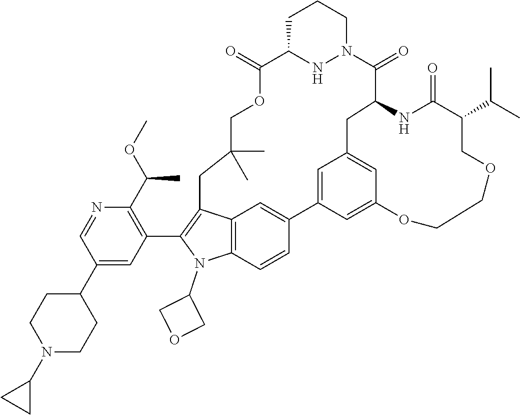 [SMILES: CO[C@@H](C)c1ncc(C2CCN(C3CC3)CC2)cc1-c1c2c3cc(ccc3n1C1COC1)-c1cc3cc(c1)OCCOC[C@@H](C(C)C)C(=O)N[C@@H](C3)C(=O)N1CCC[C@H](N1)C(=O)OCC(C)(C)C2] |
| A12 | 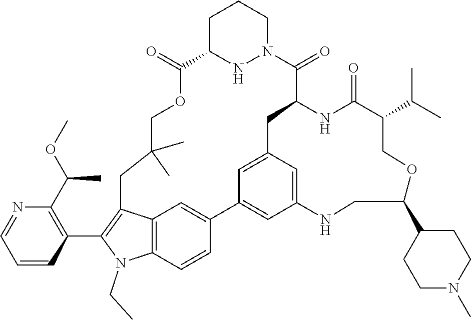 |
| A13 | 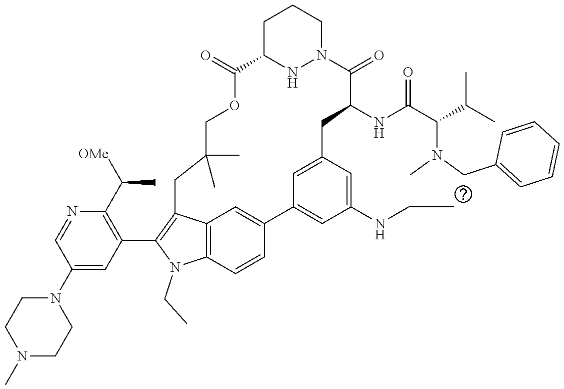 [SMILES: CCNc1cc2cc(c1)-c1ccc3c(c1)c(c(-c1cc(N4CCN(C)CC4)cnc1[C@H](C)OC)n3CC)CC(C)(C)COC(=O)[C@@H]1CCCN(N1)C(=O)[C@@H](NC(=O)[C@H](C(C)C)N(C)Cc1ccccc1)C2] |
| A14 | 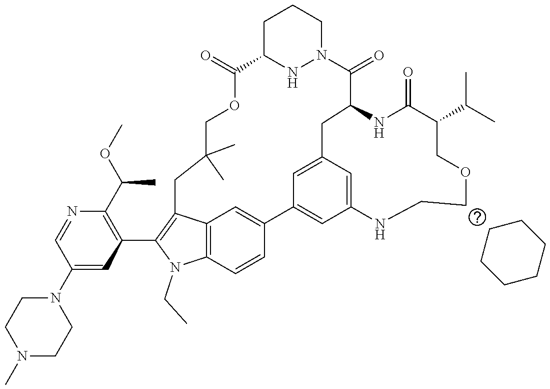 |
| A15 | 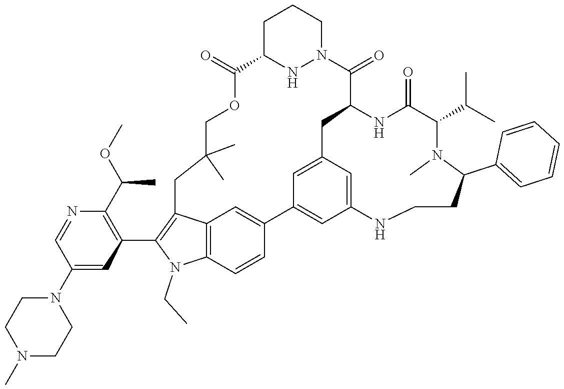 |
| A16 | 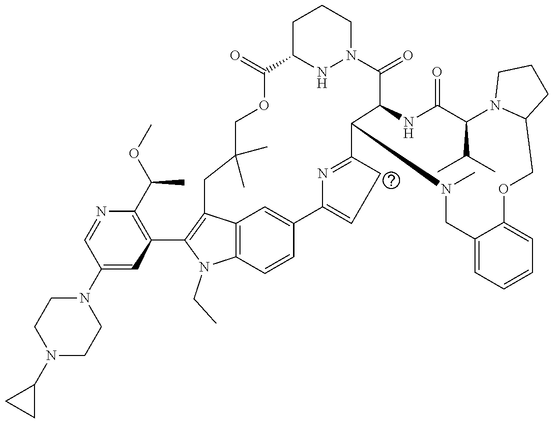 |
| A17 | 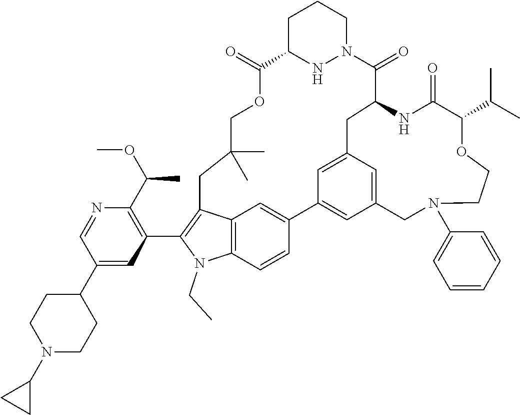 |
| A18 | 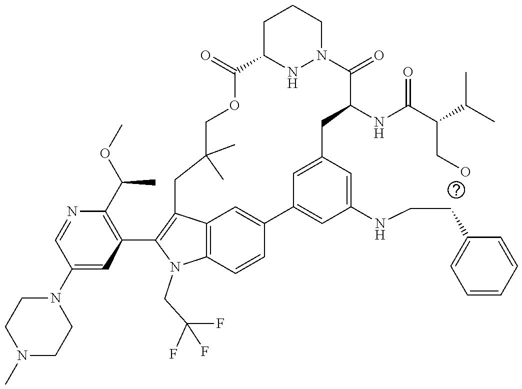 |
| A19 | 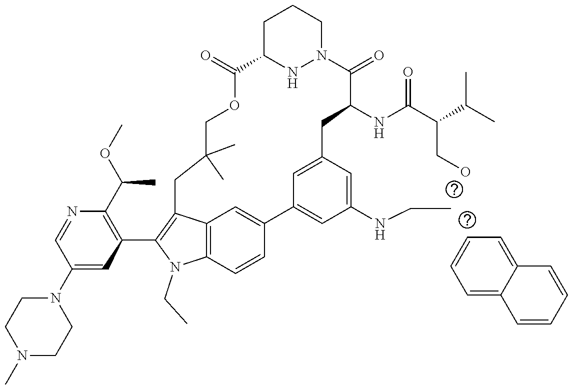 |
| A20 | 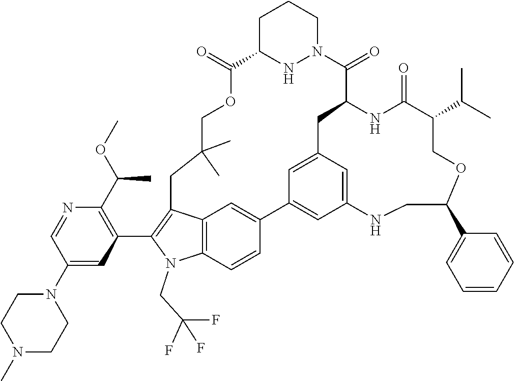 |
| A21 | 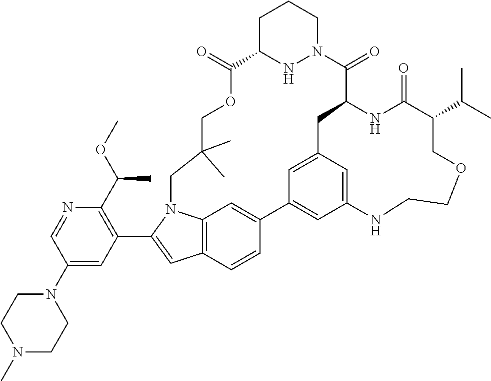 [SMILES: CO[C@@H](C)c1ncc(N2CCN(C)CC2)cc1-c1cc2ccc3cc2n1CC(C)(C)COC(=O)[C@@H]1CCCN(N1)C(=O)[C@@H]1Cc2cc(cc-3c2)NCCOC[C@@H](C(C)C)C(=O)N1] |
| A22 | 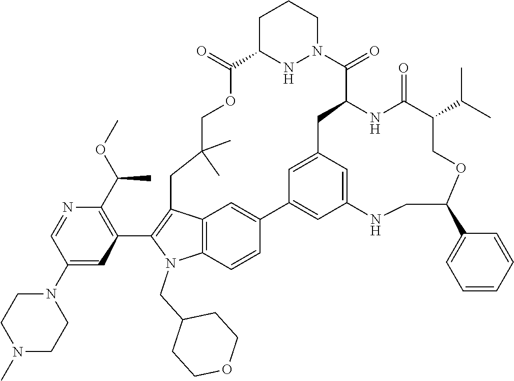 |
| A23 | 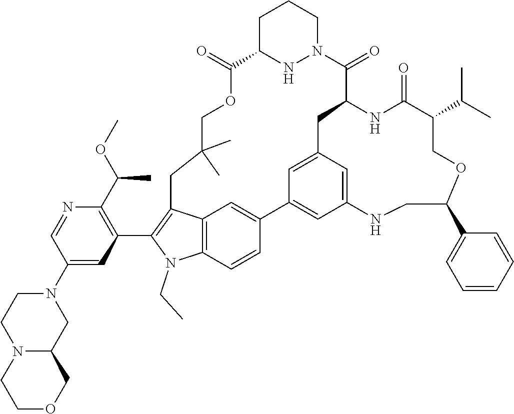 |
| A24 | 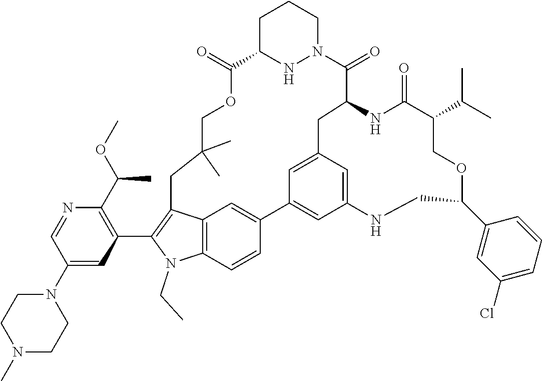 |
| A25 | 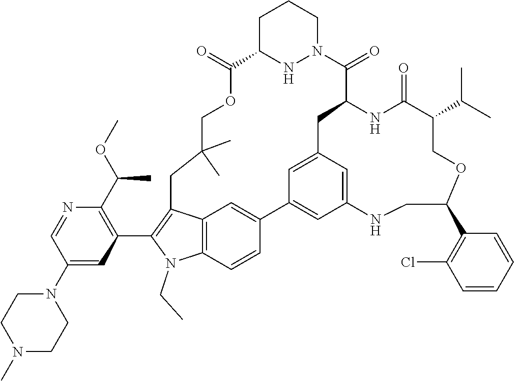 |
| A26 | 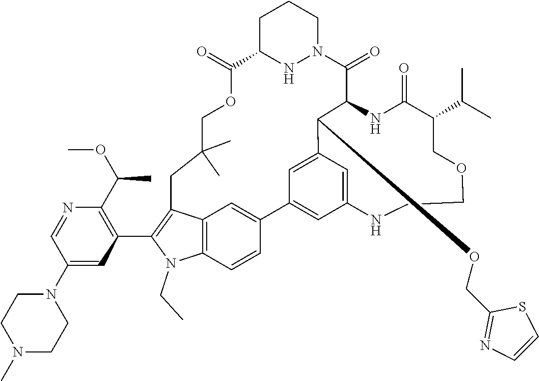 |
| A27 | 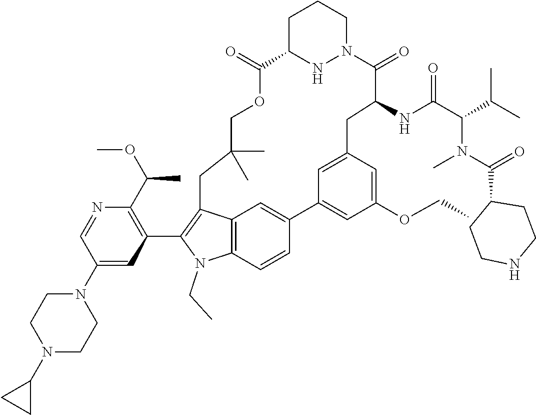 |
| A28 | 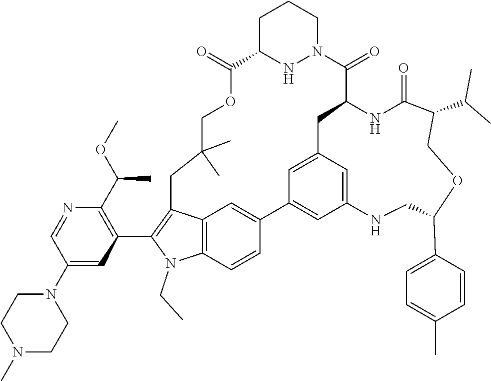 |
| A29 | 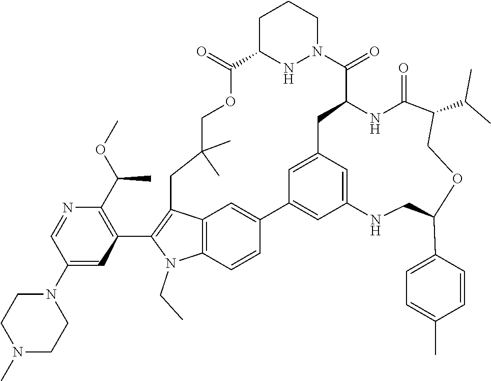 |
| A30 | 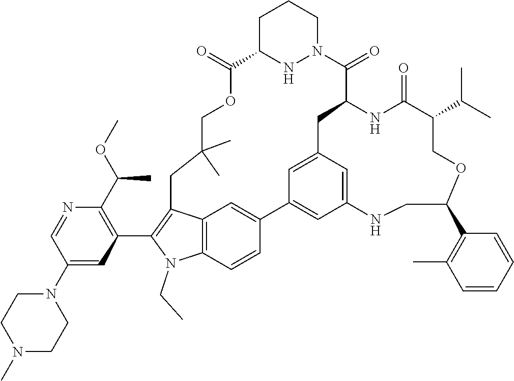 |
| A31 | 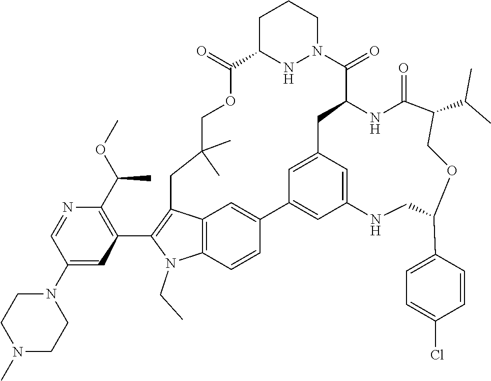 |
| A32 | 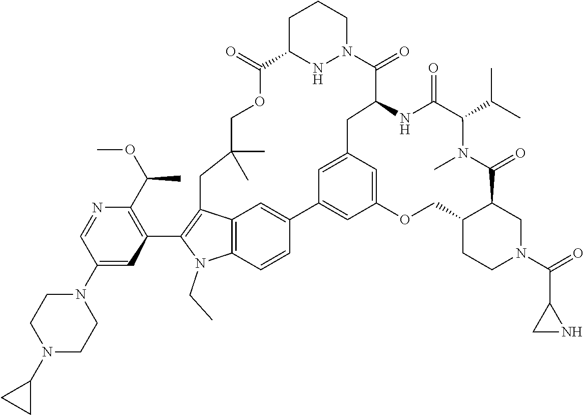 |
| | [symbol] |
| A33 | 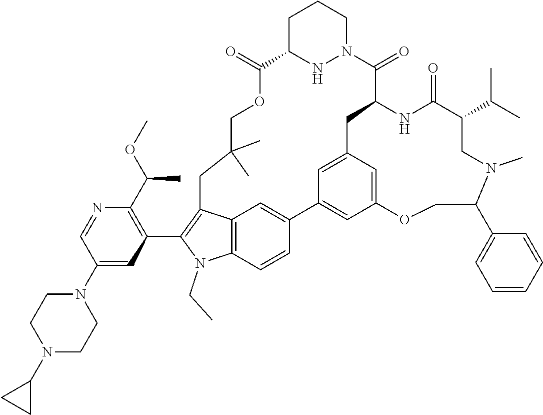 |
| A34 | 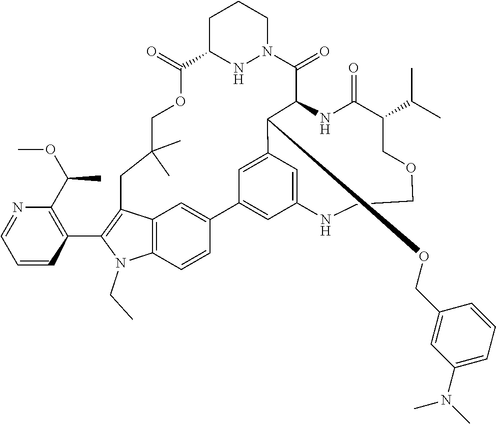 |
| A35 | 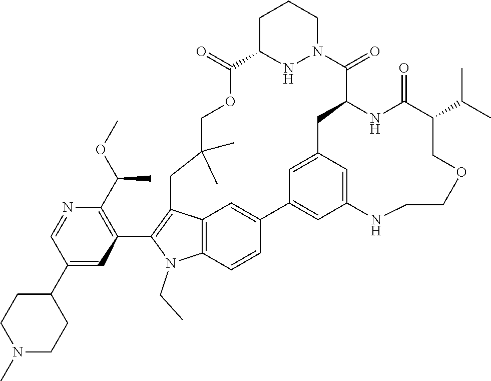 |
| A36 | 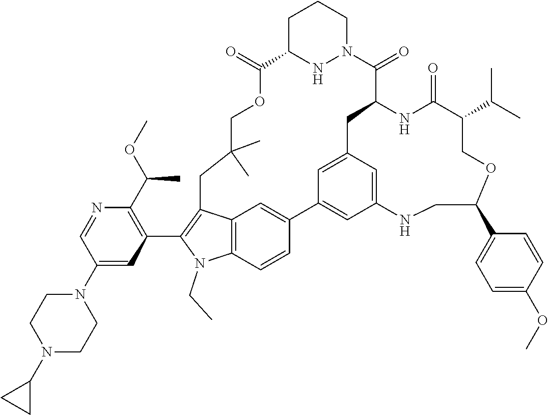 |
| | assumed |
| A37 | 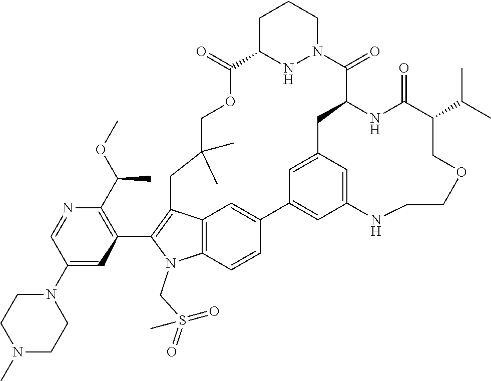 |
| A38 | 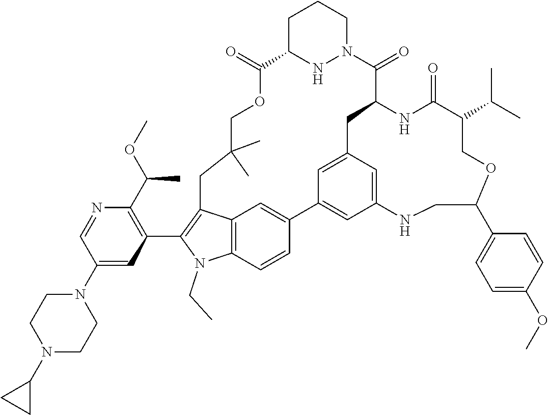 |
| A39 | 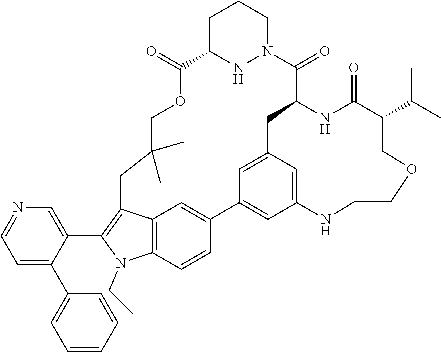 [SMILES: CCn1c(-c2cnccc2-c2ccccc2)c2c3cc(ccc31)-c1cc3cc(c1)NCCOC[C@@H](C(C)C)C(=O)N[C@@H](C3)C(=O)N1CCC[C@H](N1)C(=O)OCC(C)(C)C2] |
| A40 | 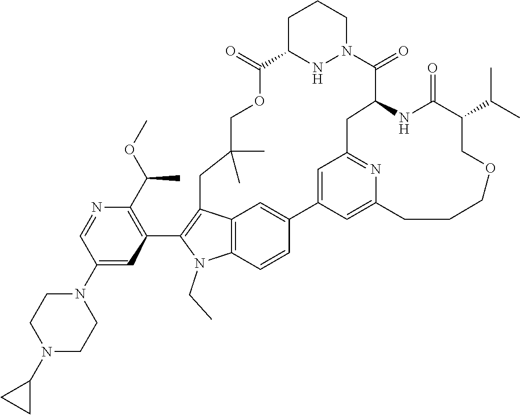 |
| A41 | 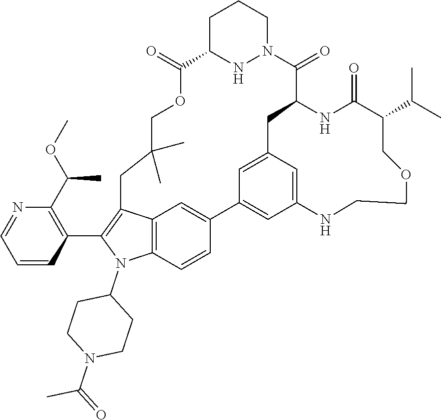 |
| A42 | 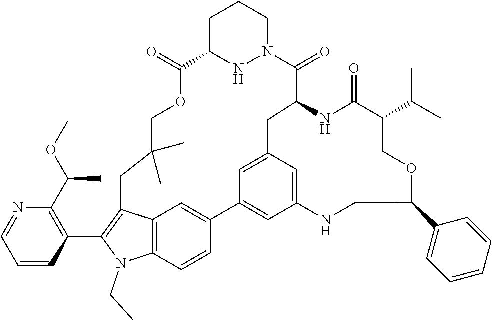 |
| A43 | 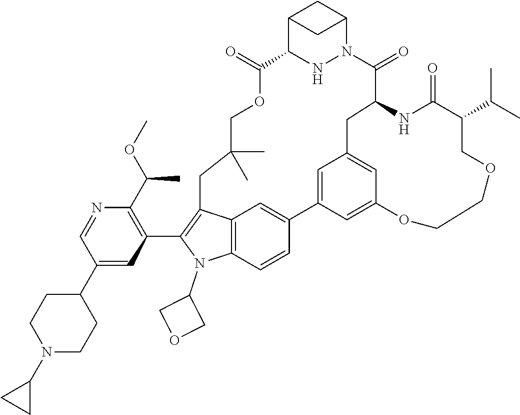 |
| A44 | 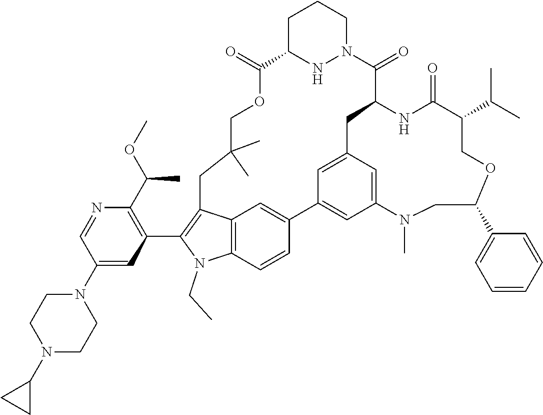 |
| A45 | 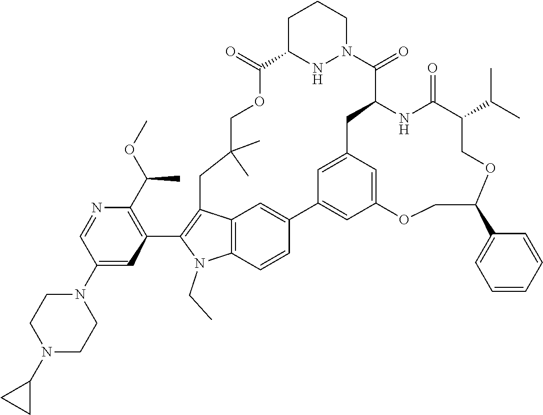 |
| A46 | 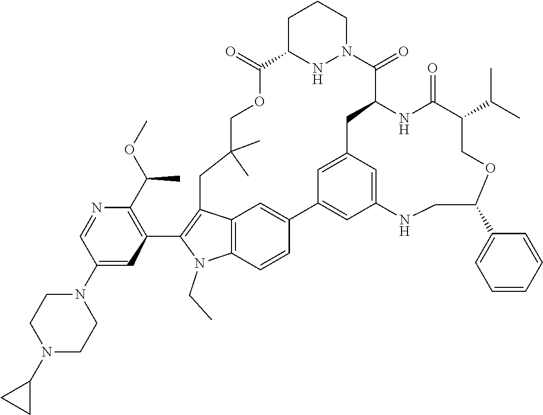 |
| A47 | 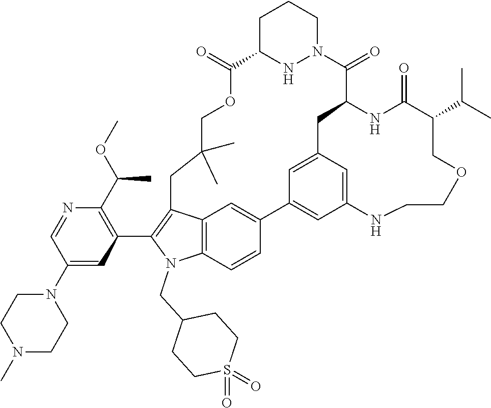 |
| A48 | 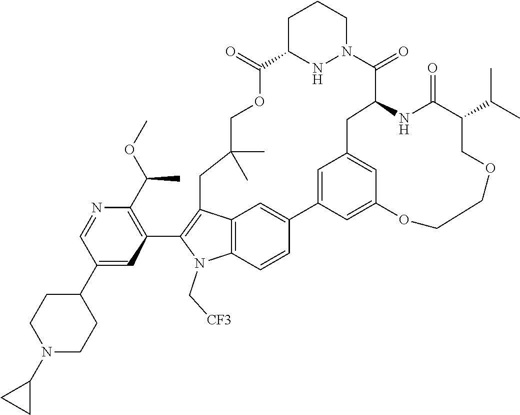 |
| A49 | 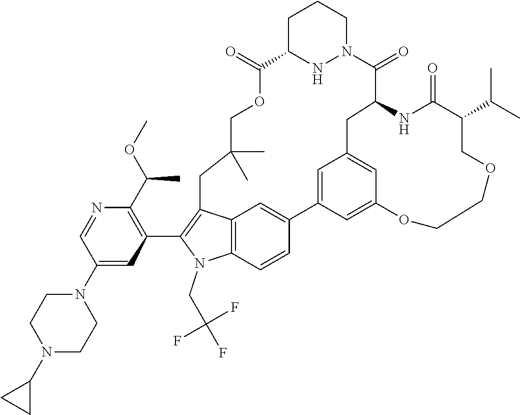 |
| A50 | 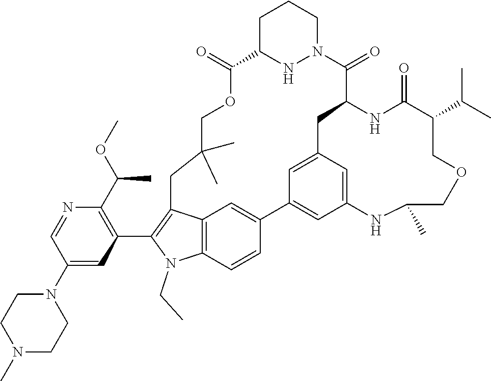 |
| A51 | 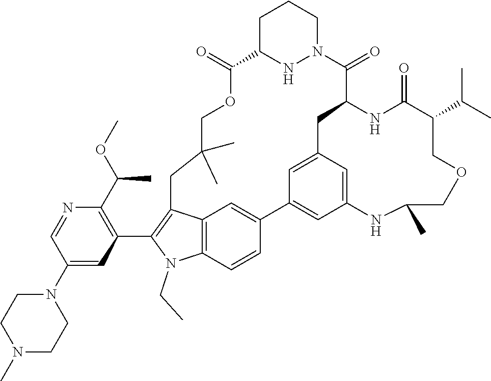 |
| A52 | 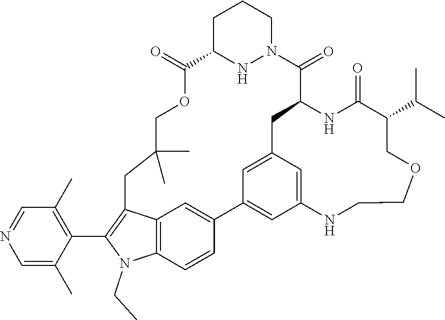 [SMILES: CCn1c(-c2c(C)cncc2C)c2c3cc(ccc31)-c1cc3cc(c1)NCCOC[C@@H](C(C)C)C(=O)N[C@@H](C3)C(=O)N1CCC[C@H](N1)C(=O)OCC(C)(C)C2] |
| A53 | 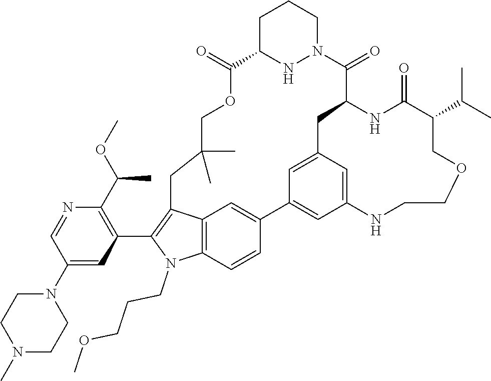 |
| A54 | 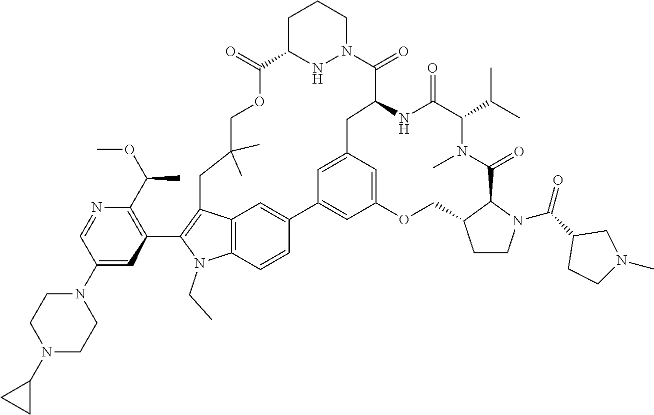 |
| A55 | 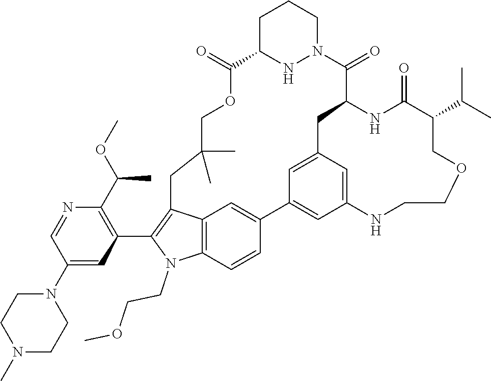 |
| A56 | 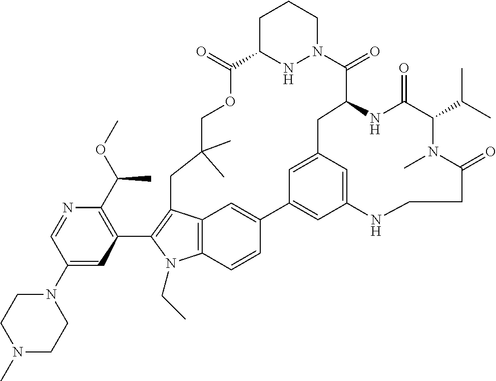 |
| A57 | 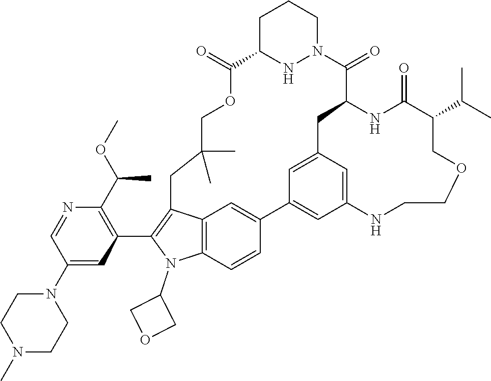 |
| A58 | 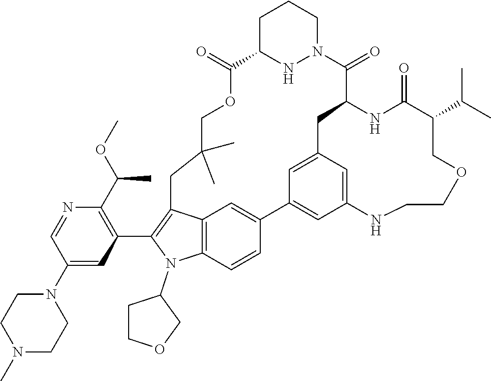 |
| A59 | 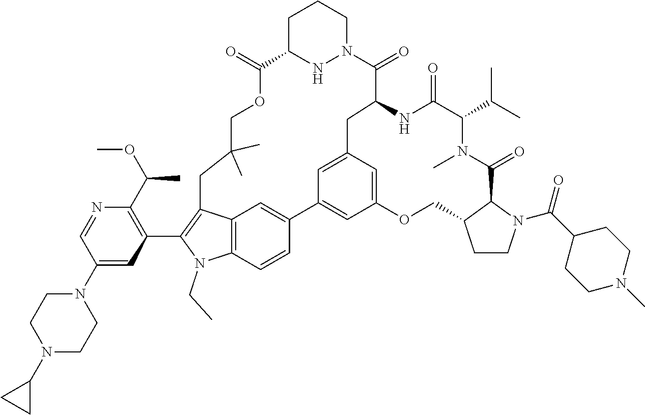 |
| A60 | 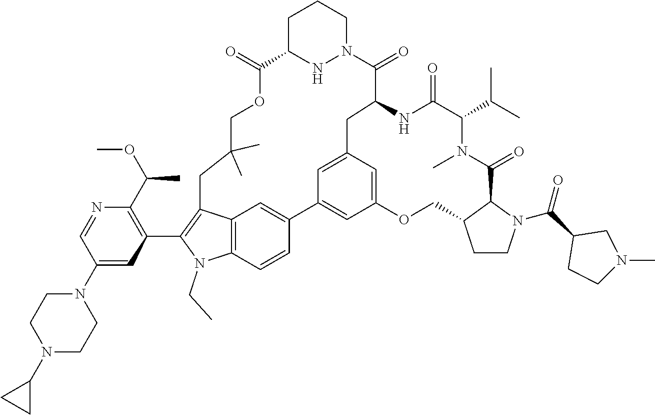 |
| A61 | 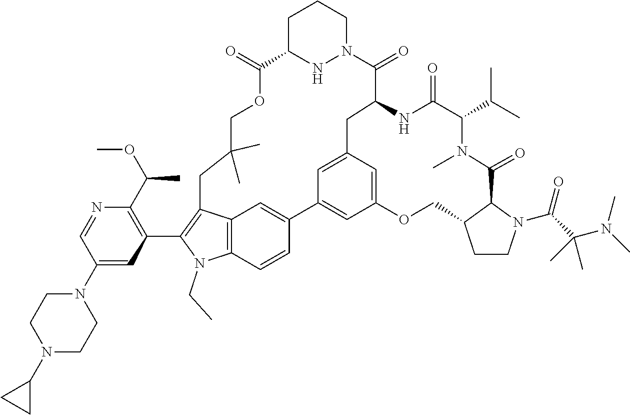 |
| A62 | 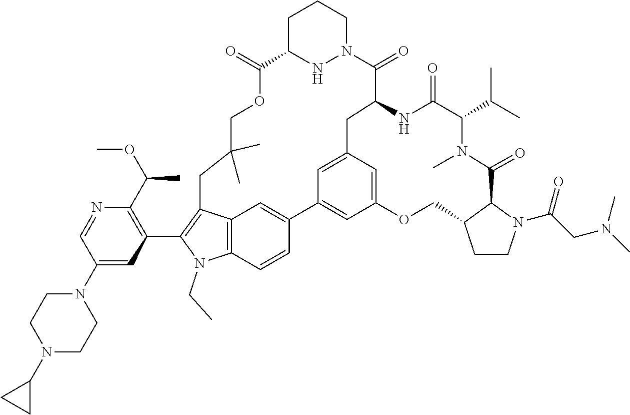 |
| A63 | 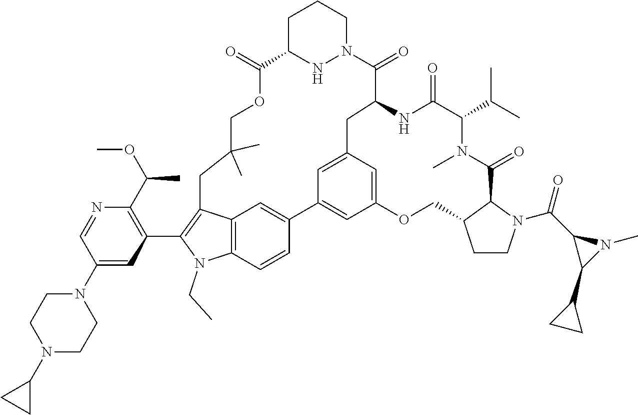 |
| A64 | 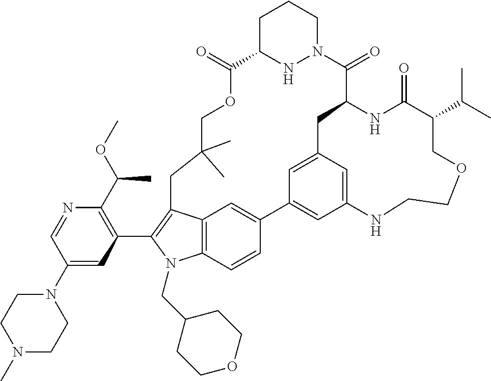 |
| A65 | 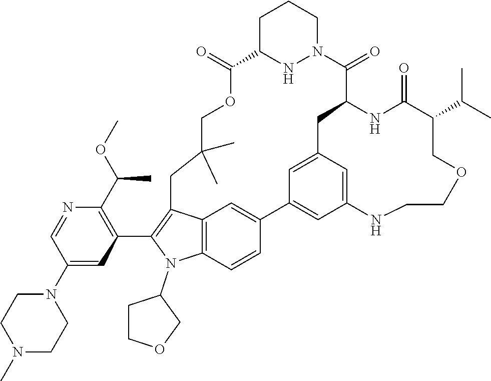 |
| A66 | 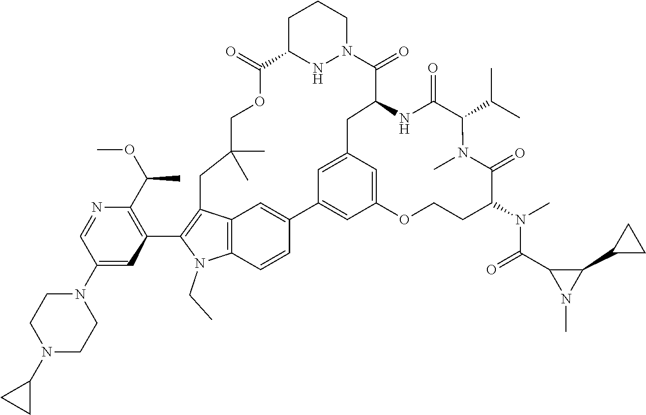 |
| A67 | 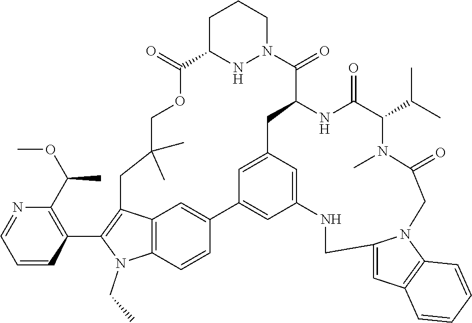 |
| A68 | 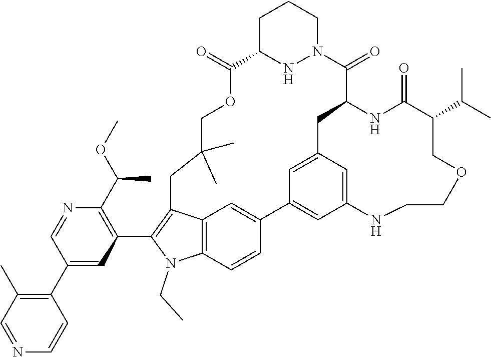 |
| A69 | 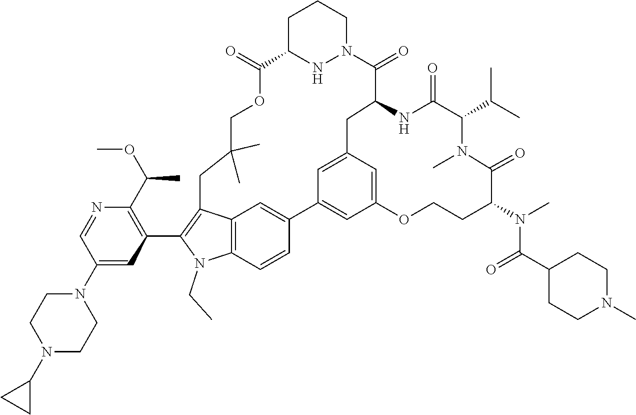 |
| A70 | 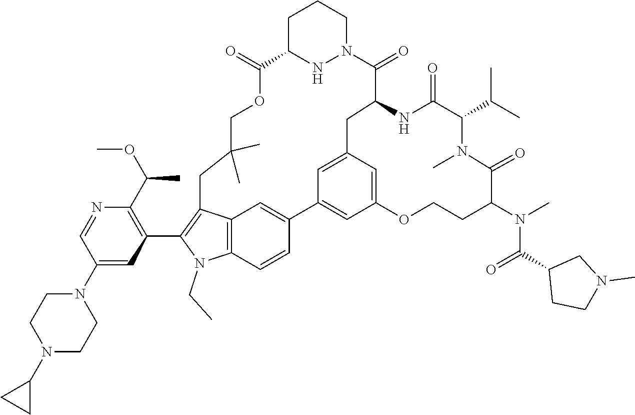 |
| A71 | 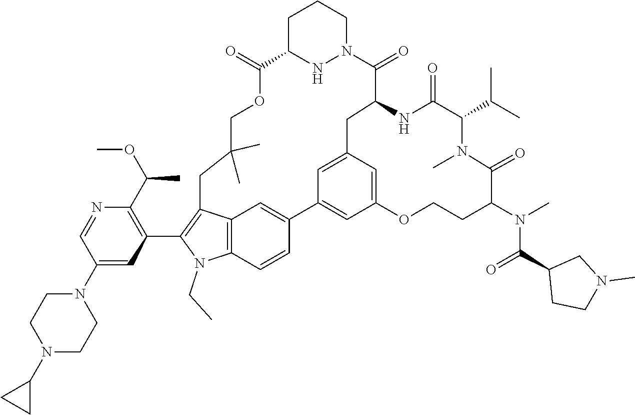 |
| A72 | 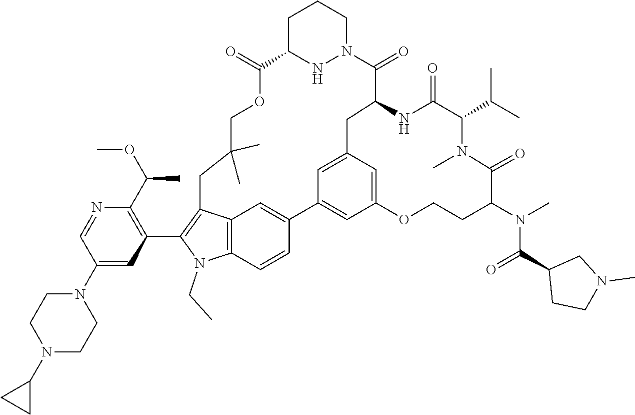 |
| A73 | 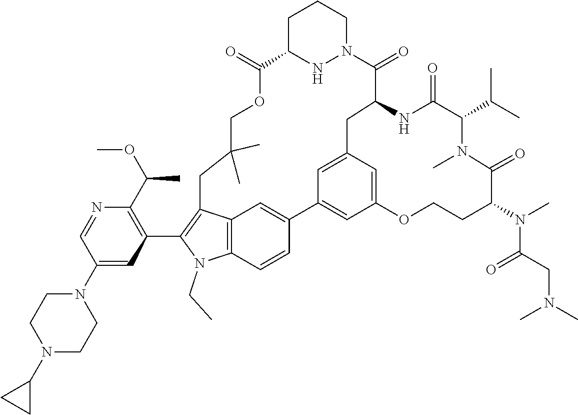 |
| A75 | 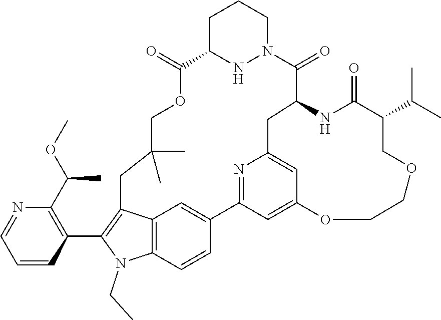 |
| A76 | 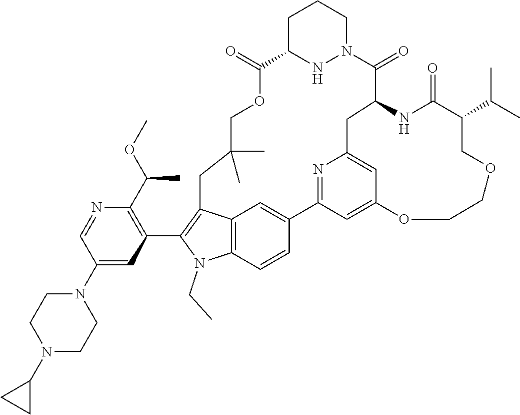 |
| A77 | 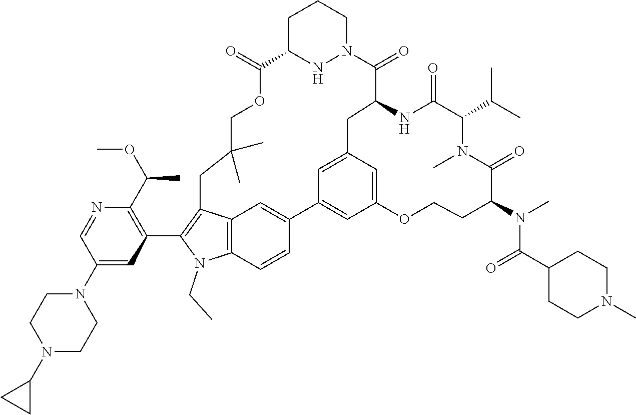 |
| A78 | 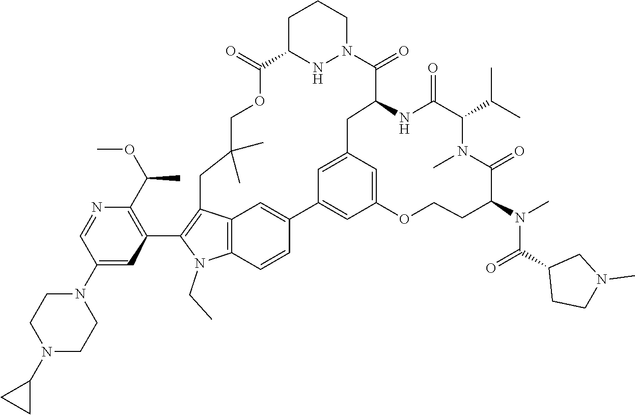 |
| A79 | 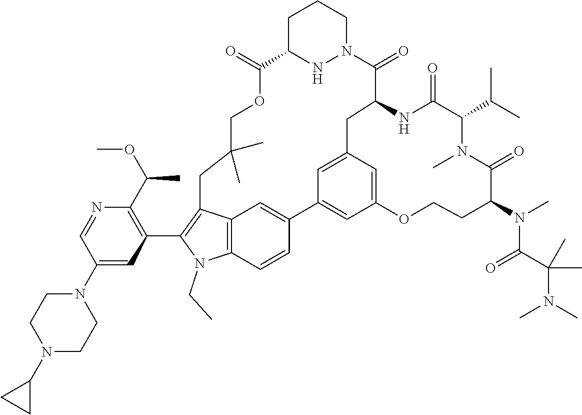 |
| A80 | 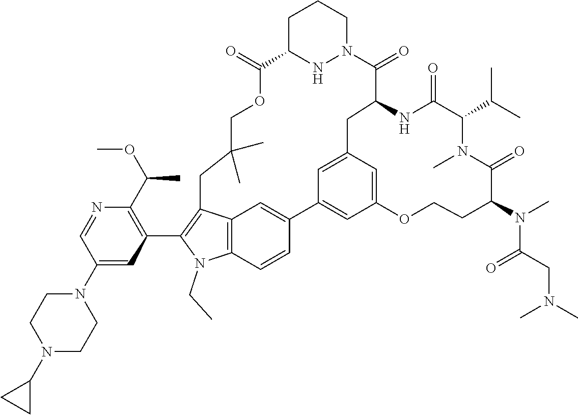 |
| A81 | 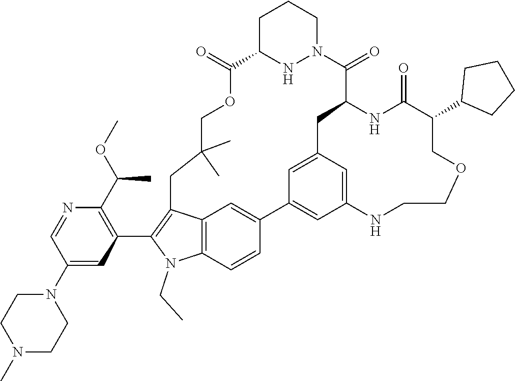 |
| A82 | 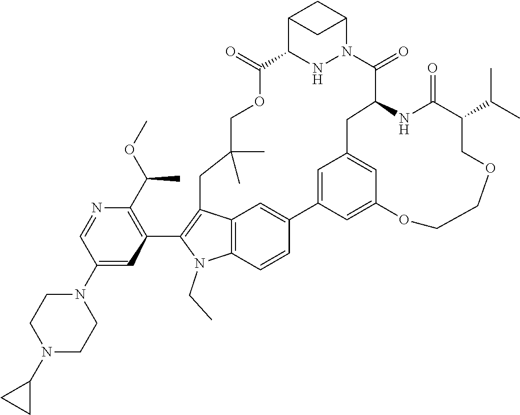 |
| A83 | 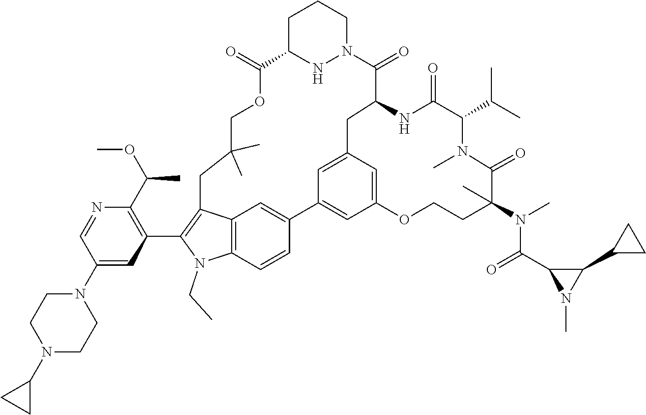 |
| A84 | 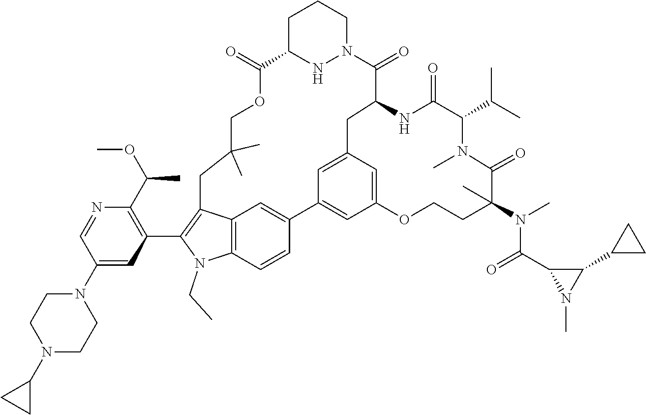 |
| A85 | 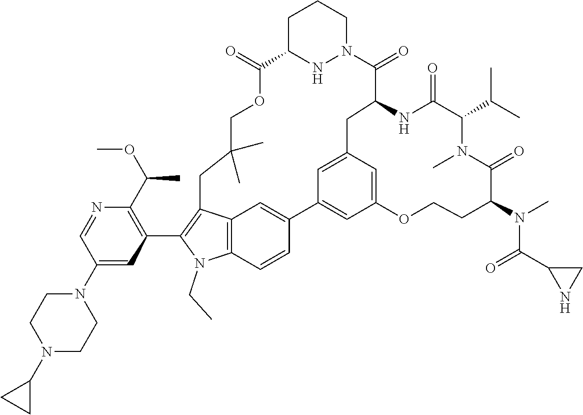 |
| A86 | 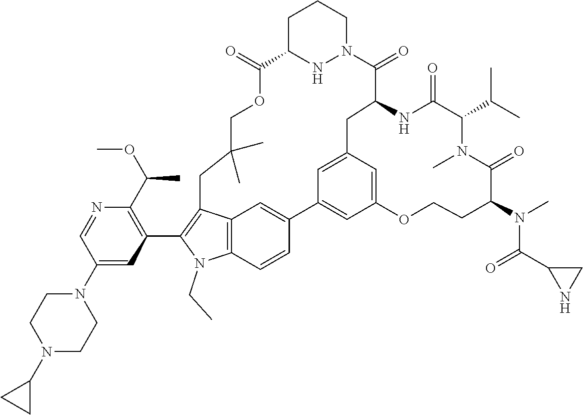 |
| A87 | 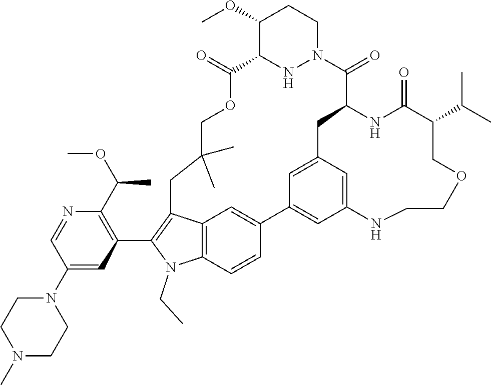 |
| A88 | 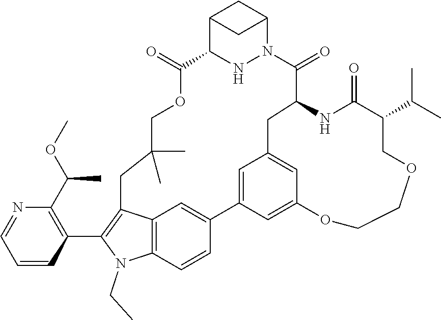 |
| A89 | 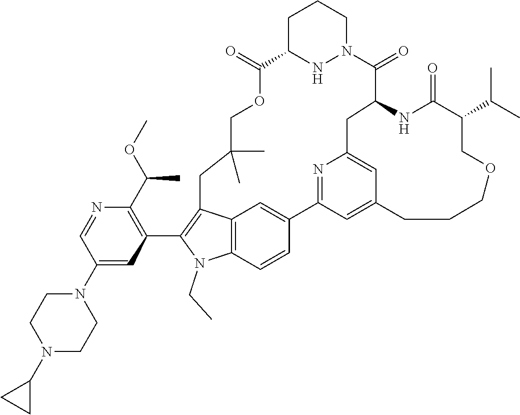 |
| A90 | 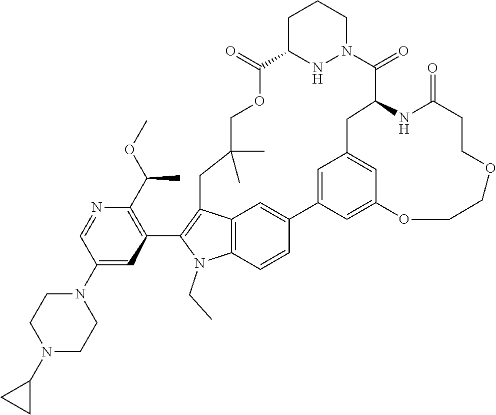 |
| A91 | 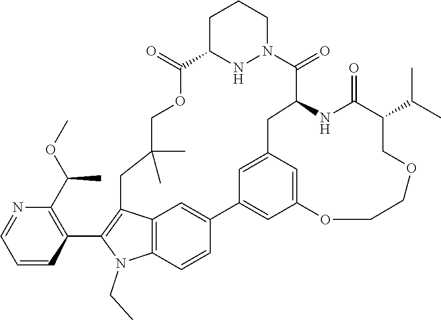 |
| A92 | 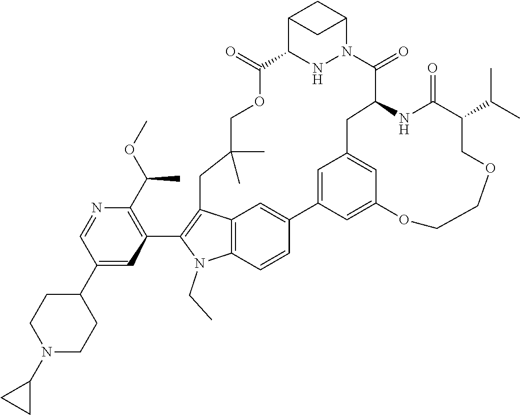 |
| A93 | 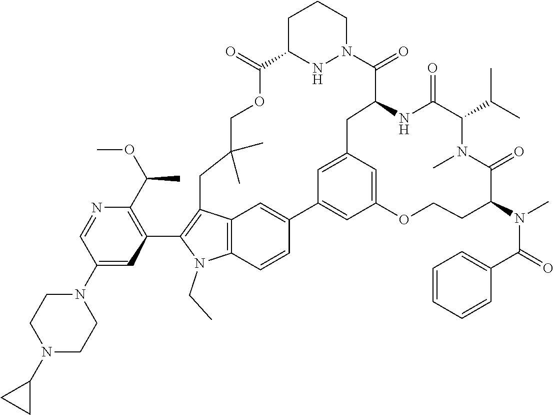 |
| A94 | 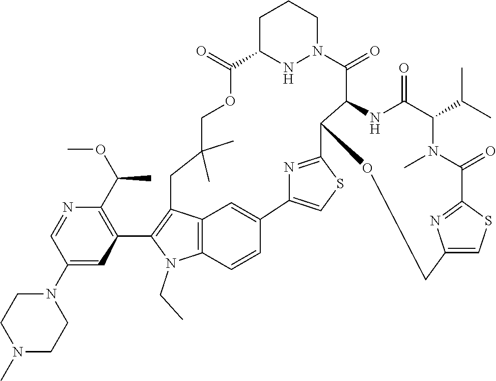 |
| A95 | 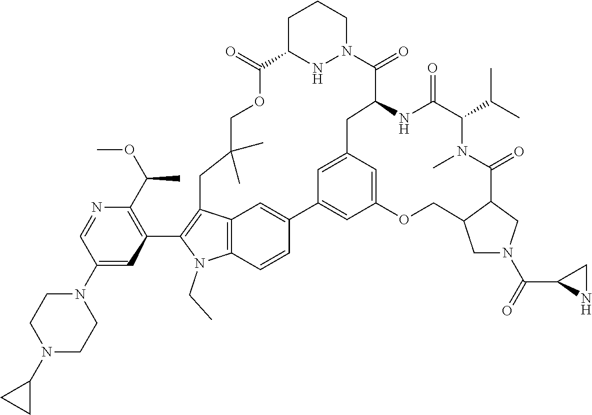 |
| A96 | 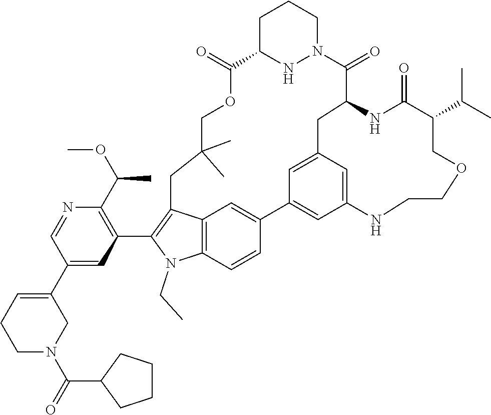 |
| A97 | 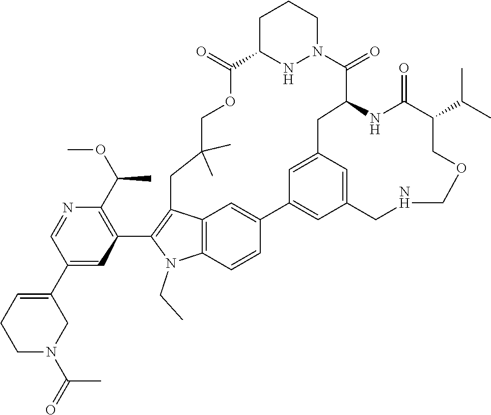 |
| A98 | 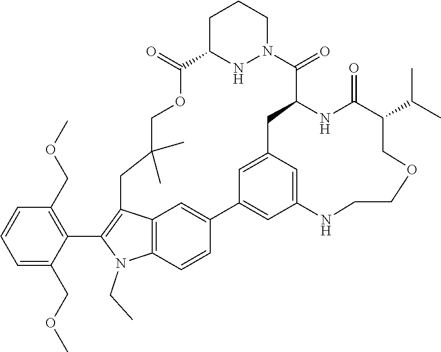 [SMILES: CCn1c(-c2c(COC)cccc2COC)c2c3cc(ccc31)-c1cc3cc(c1)NCCOC[C@@H](C(C)C)C(=O)N[C@@H](C3)C(=O)N1CCC[C@H](N1)C(=O)OCC(C)(C)C2] |
| A99 | 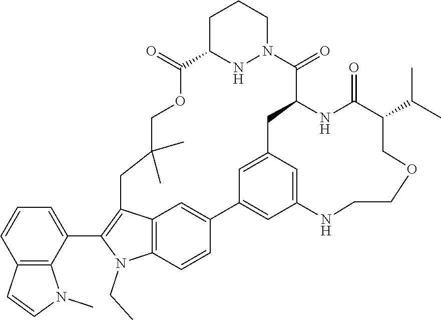 [SMILES: CCn1c(-c2cccc3ccn(C)c23)c2c3cc(ccc31)-c1cc3cc(c1)NCCOC[C@@H](C(C)C)C(=O)N[C@@H](C3)C(=O)N1CCC[C@H](N1)C(=O)OCC(C)(C)C2] |
| A100 | 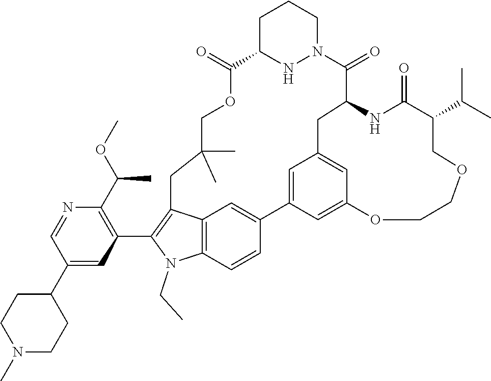 |
| A101 | 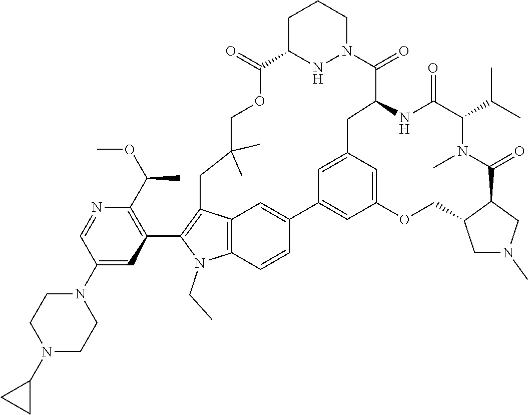 |
| A102 | 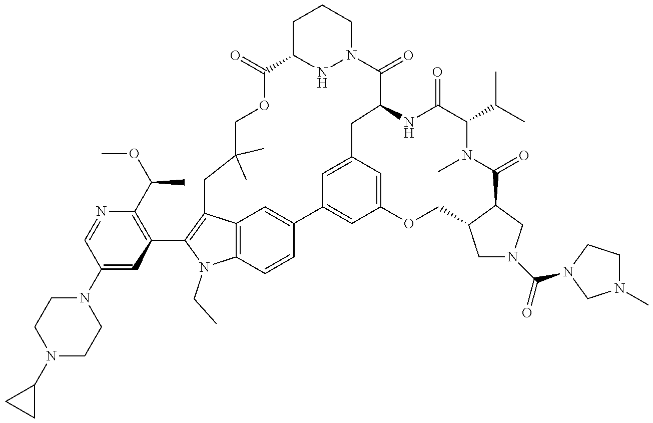 |
| A103 | 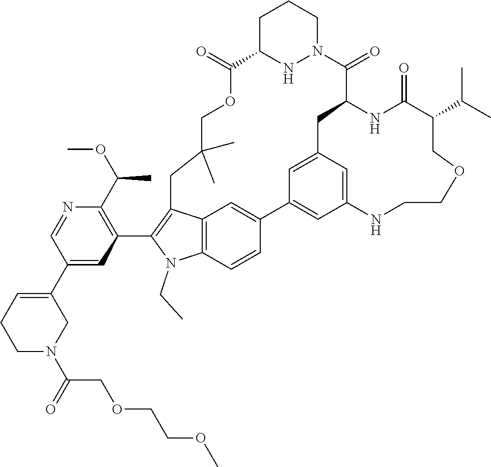 |
| A104 | 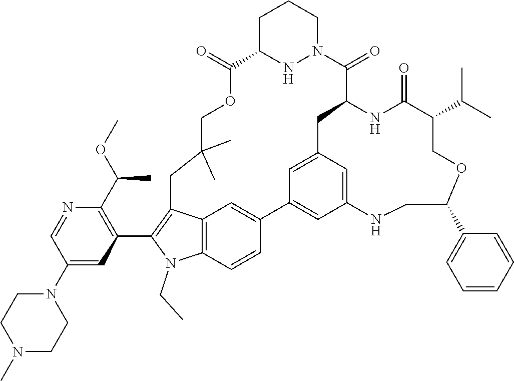 |
| A105 | 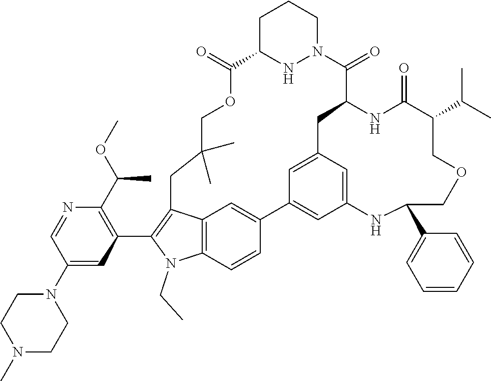 |
| A106 | 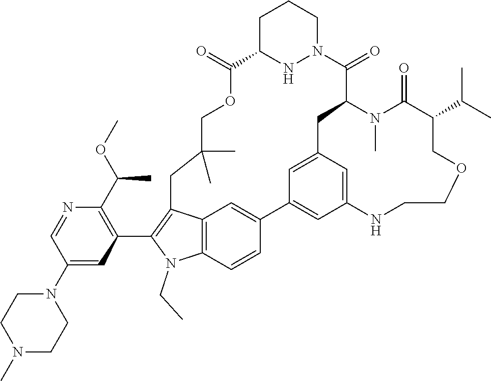 |
| A107 | 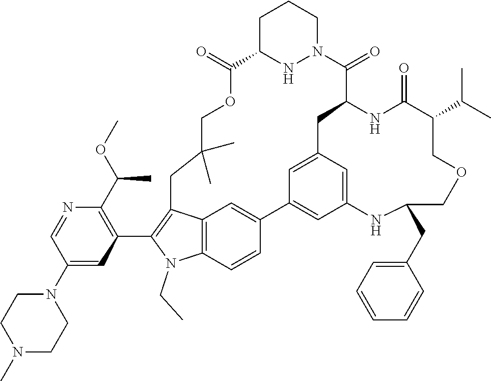 |
| A108 | 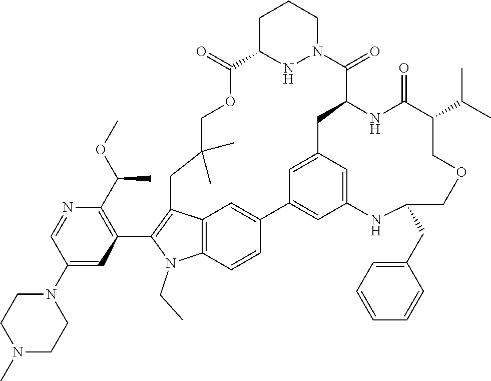 |
| A109 | 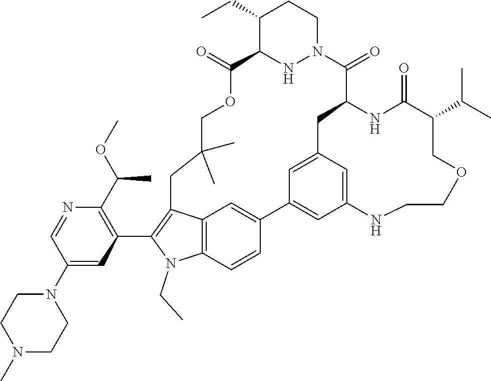 |
| A110 | 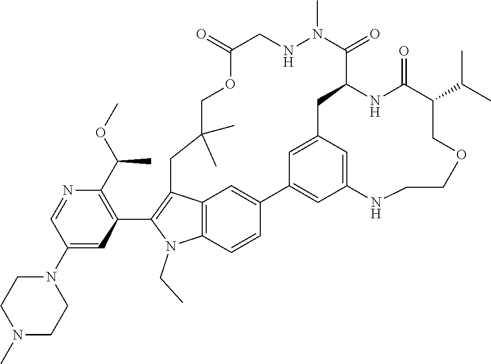 |
| A111 | 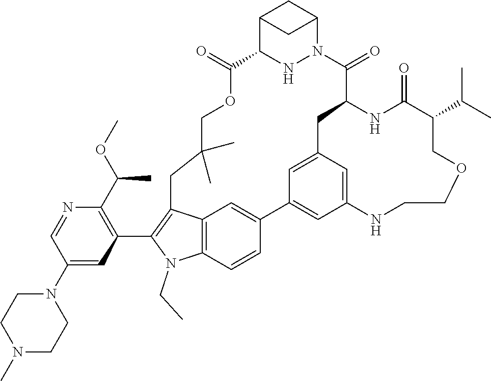 |
| A112 | 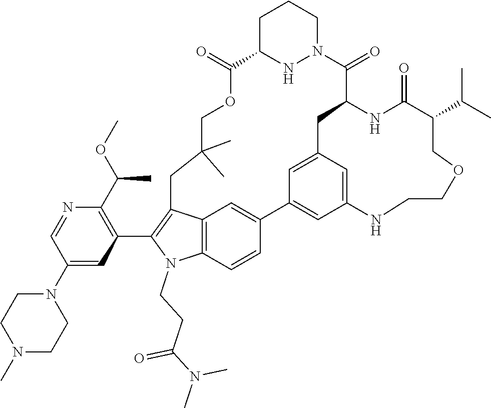 |
| A113 | 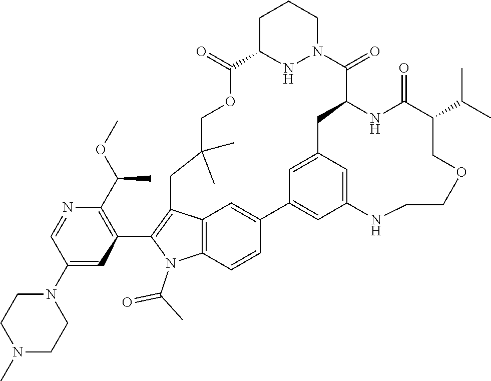 |
| A114 | 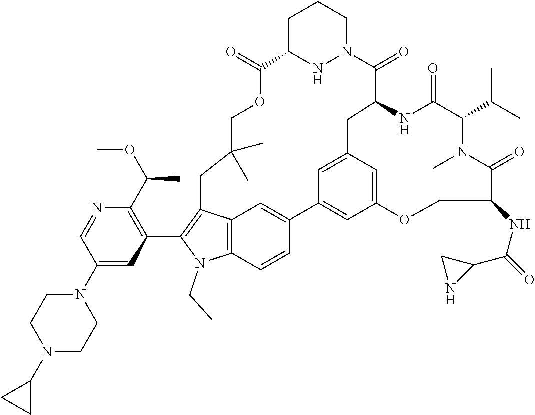 |
| A115 | 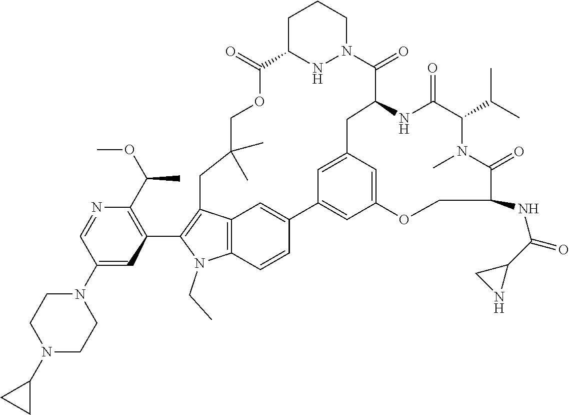 |
| A116 | 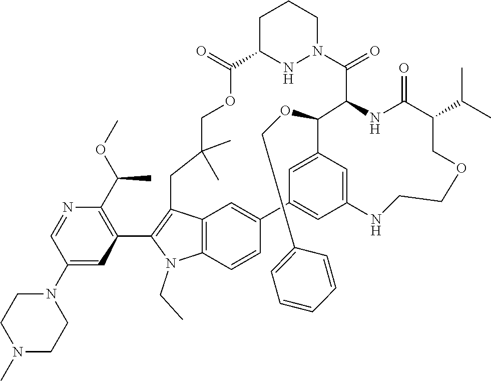 |
| A117 | 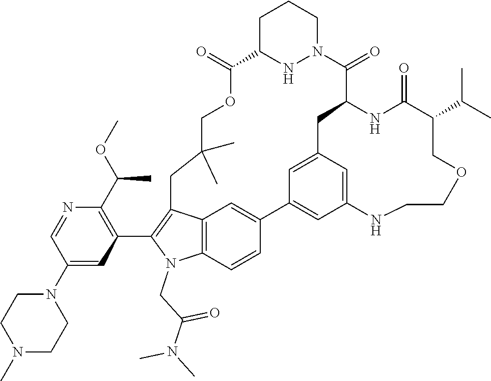 |
| A118 | 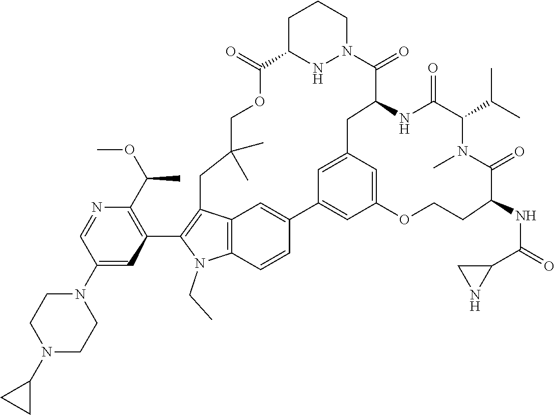 |
| A119 | 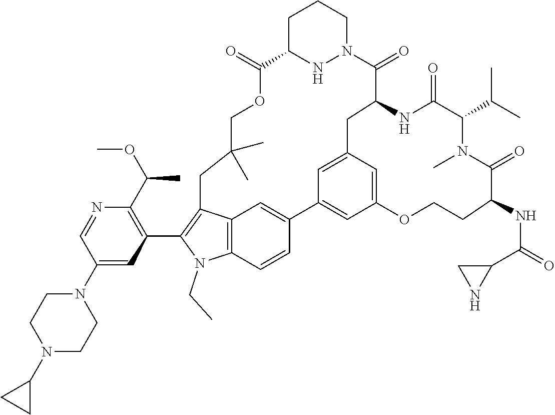 |
| A120 | 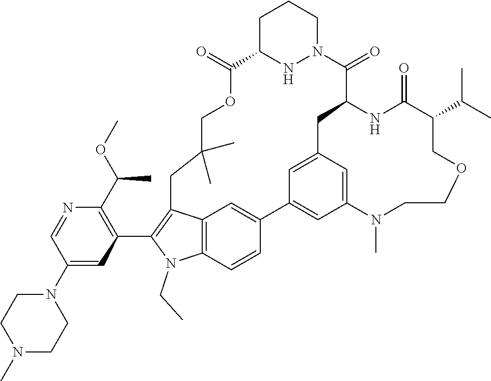 |
| A121 | 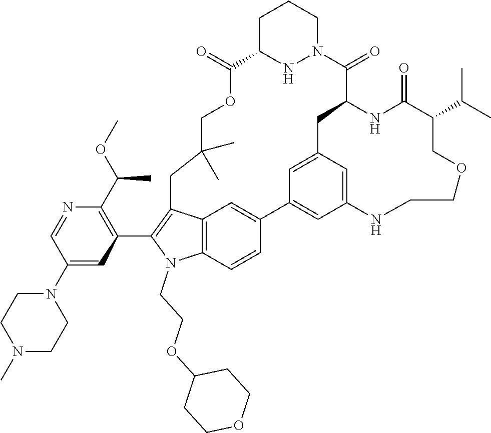 |
| A122 | 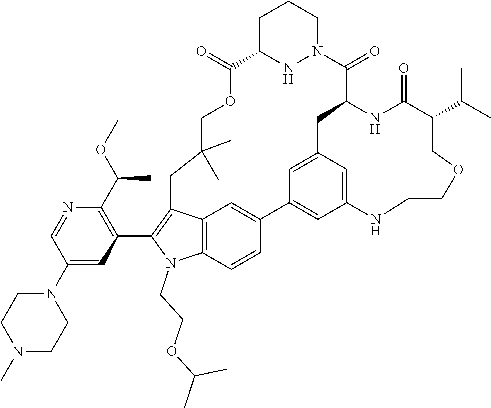 |
| A123 | 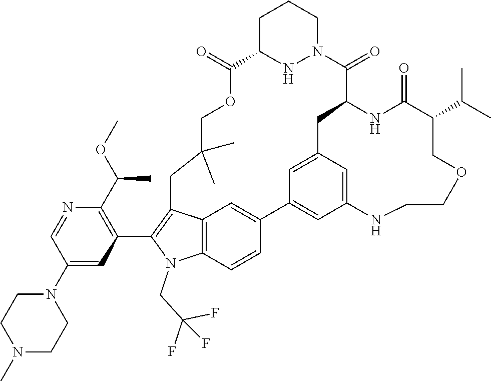 |
| A124 | 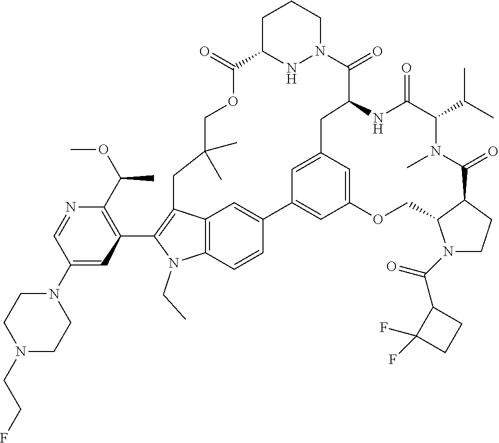 |
| A125 | 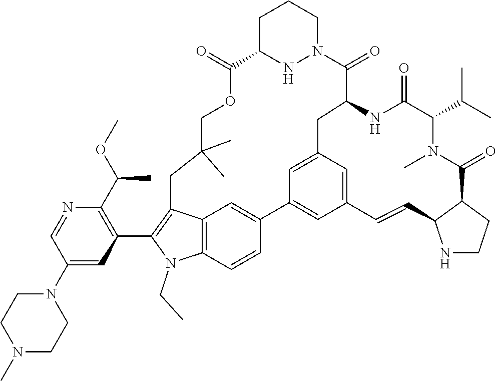 |
| A126 | 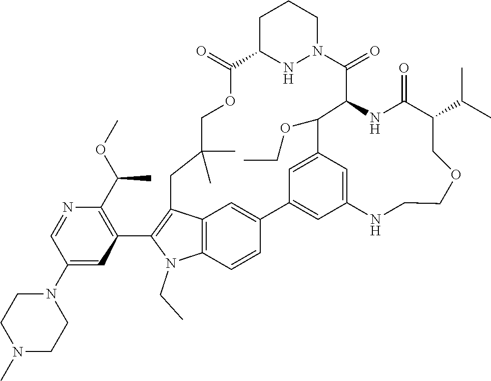 |
| A127 | 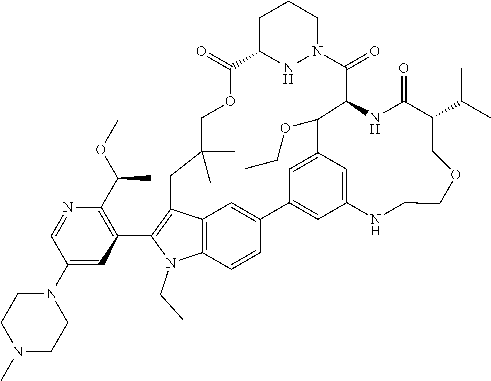 |
| A128 | 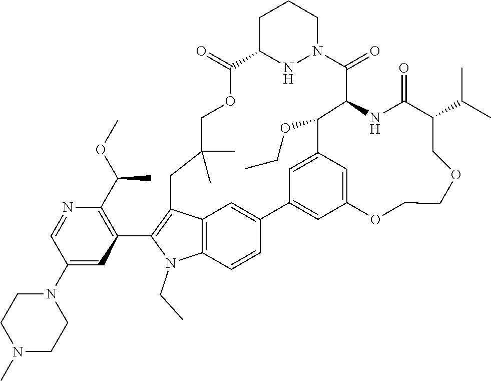 |
| A129 | 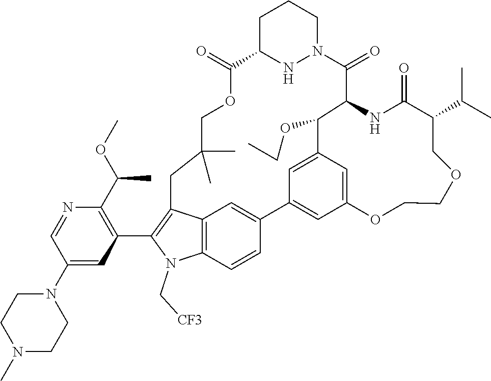 |
| A130 | 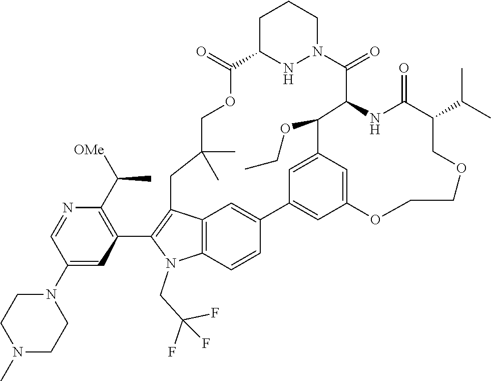 |
| A131 | 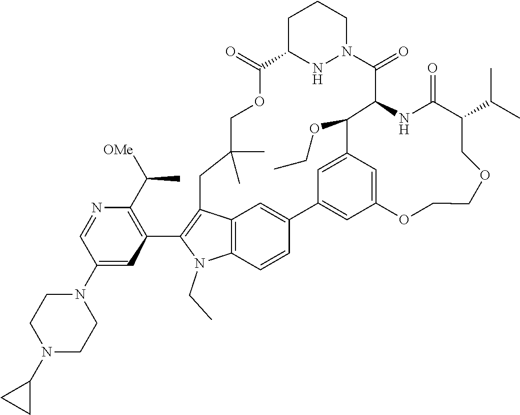 |
| A132 | 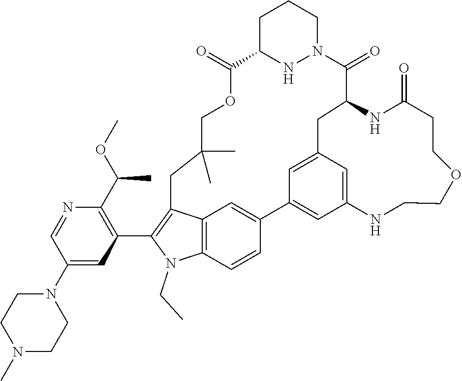 |
| A133 | 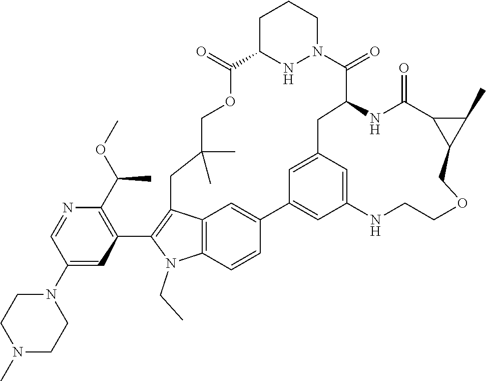 |
| A134 | 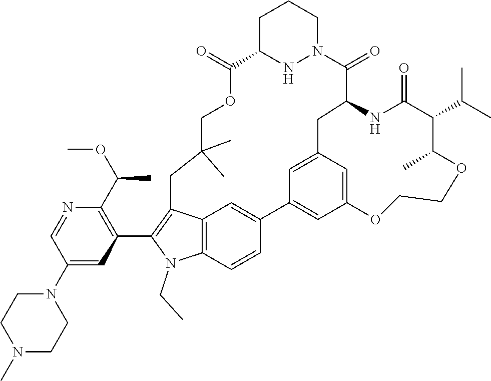 |
| A135 | 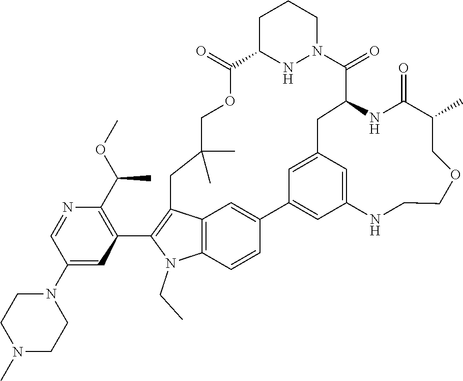 |
| A136 | 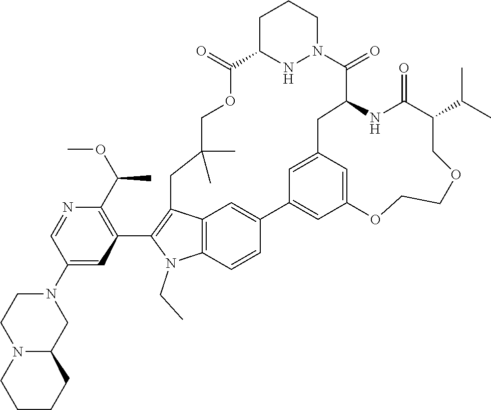 |
| A137 | 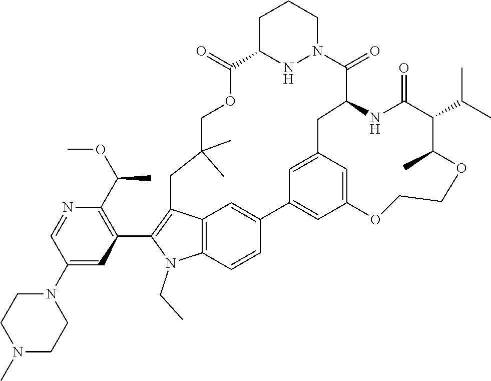 |
| A138 | 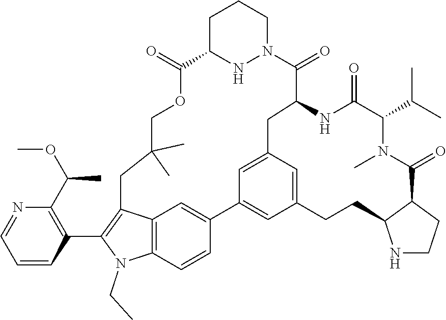 |
| A139 | 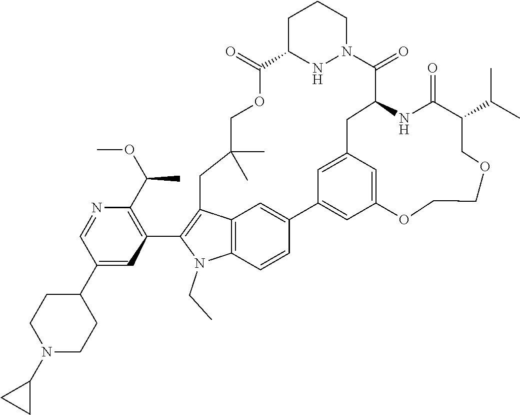 |
| A140 | 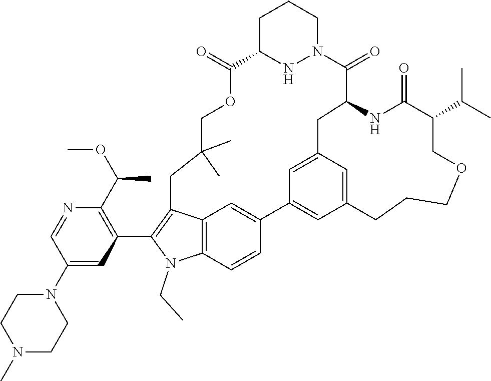 |
| A141 | 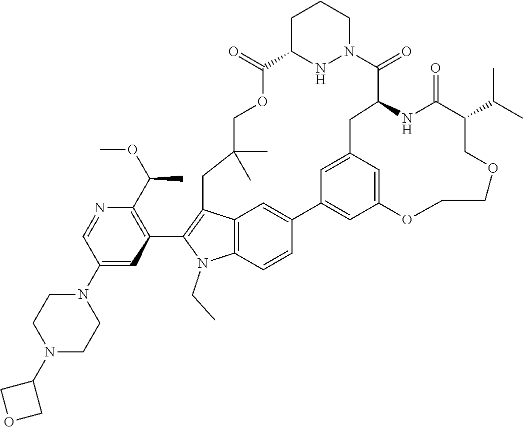 |
| A142 | 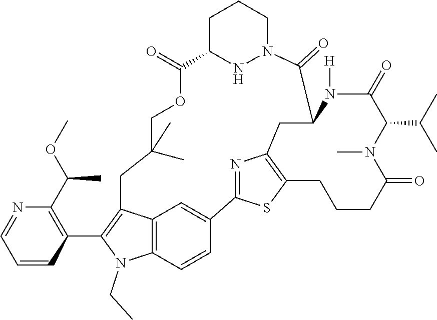 |
| A143 | 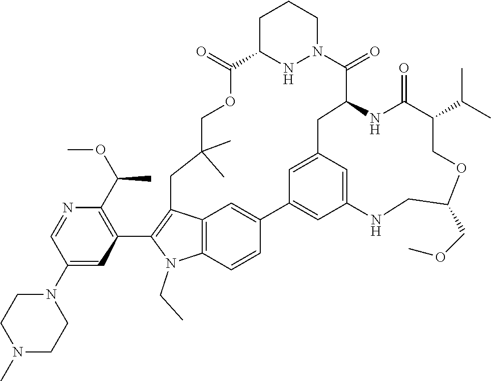 |
| A144 | 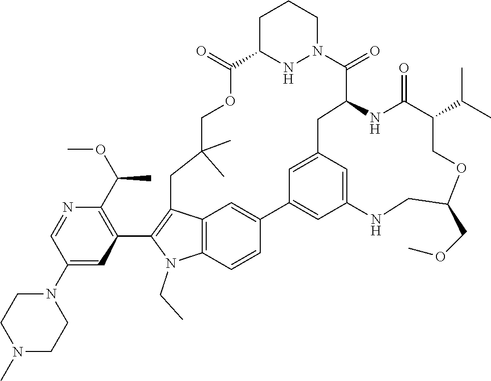 |
| A145 | 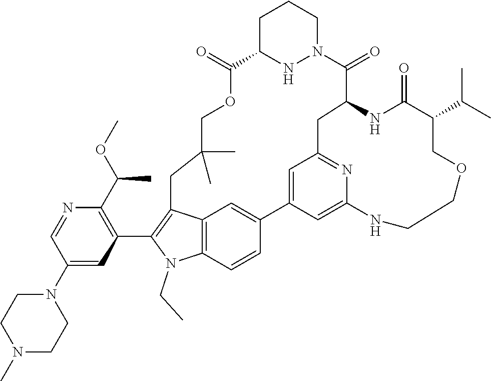 |
| A146 | 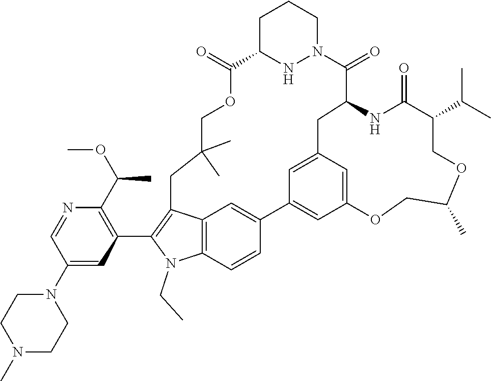 |
| A148 | 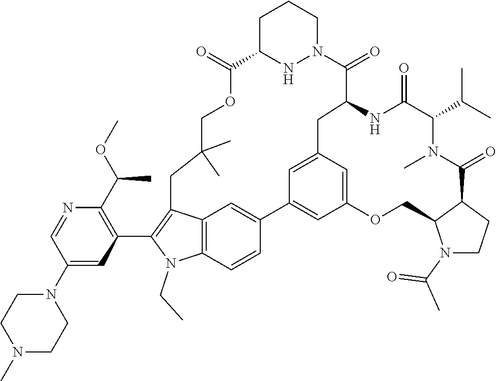 |
| A149 | 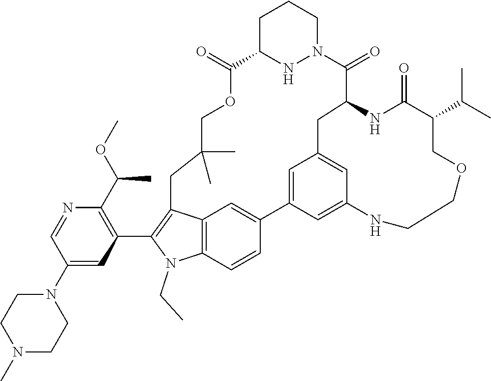 |
| A150 | 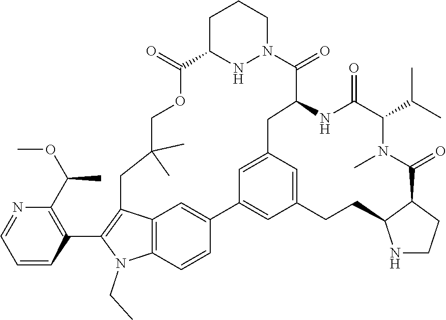 |
| A151 | 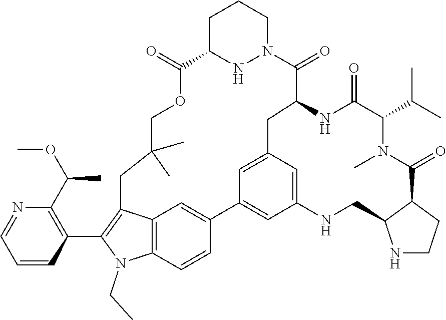 |
| A152 | 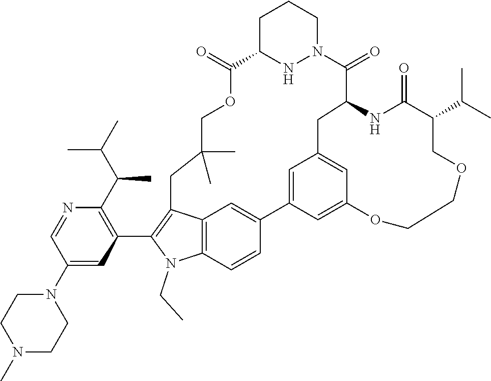 |
| A153 | 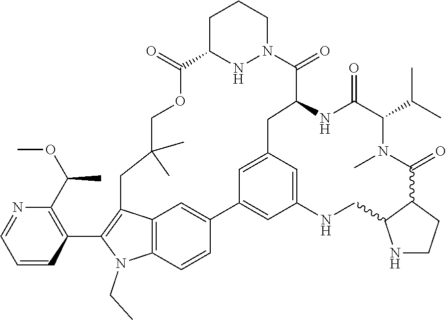 |
| A154 | 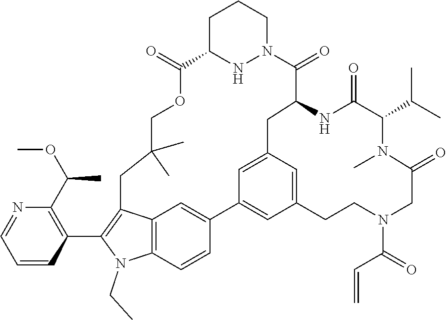 |
| A155 | 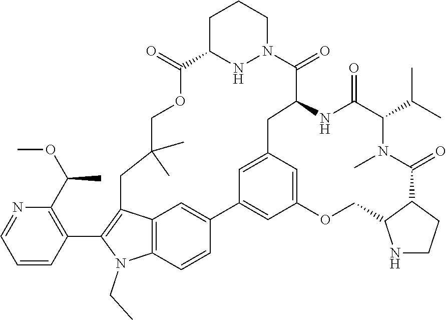 |
| A156 | 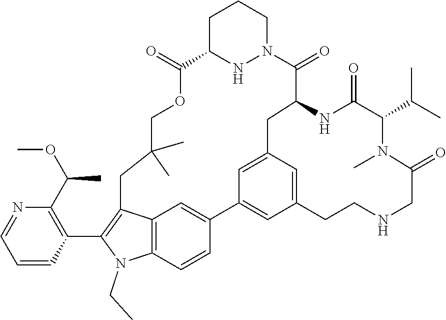 |
| A157 | 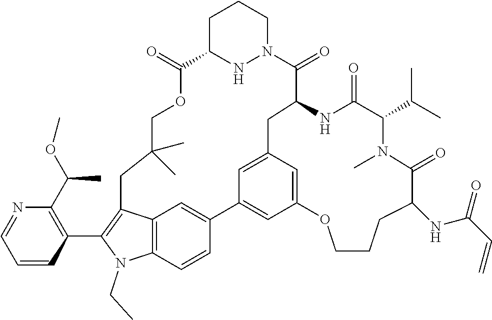 |
| A158 | 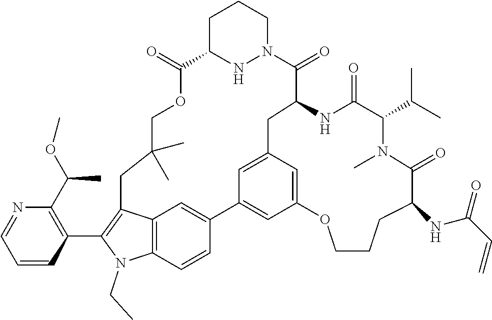 |
| A159 | 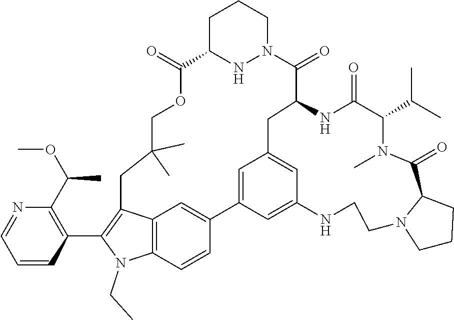 |
| A160 | 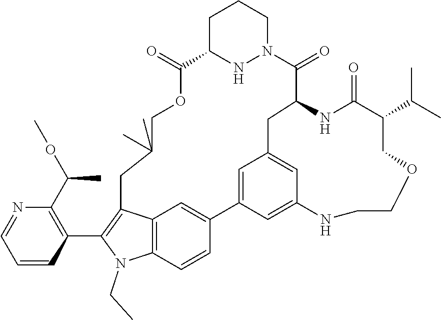 |
| A161 | 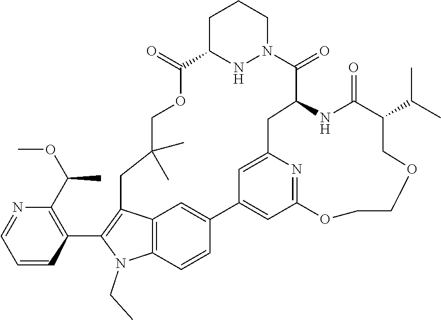 |
| A162 | 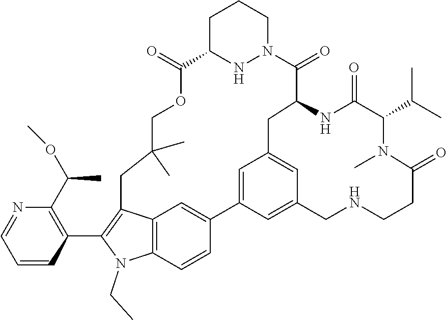 |
| A163 | 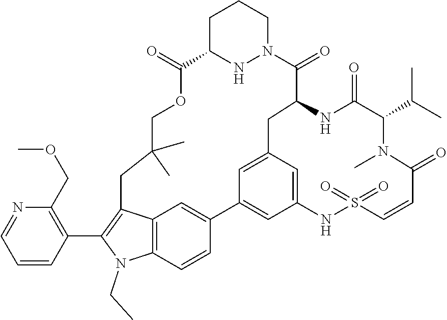 [SMILES: CCn1c(-c2cccnc2COC)c2c3cc(ccc31)-c1cc3cc(c1)NS(=O)(=O)/C=C\C(=O)N(C)[C@@H](C(C)C)C(=O)N[C@@H](C3)C(=O)N1CCC[C@H](N1)C(=O)OCC(C)(C)C2] |
| A164 | 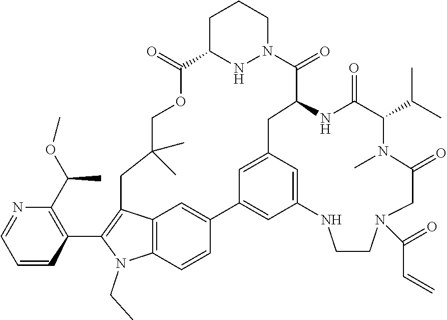 |
| A165 | 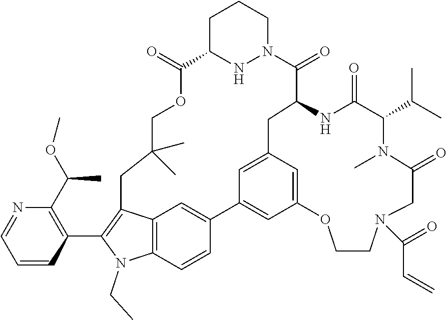 |
| A166 | 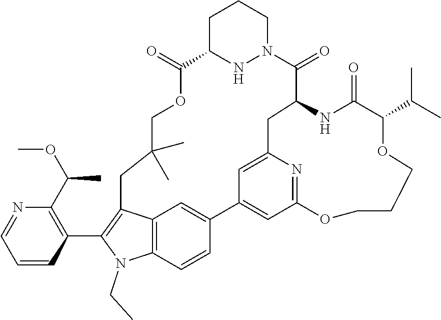 |
| A167 | 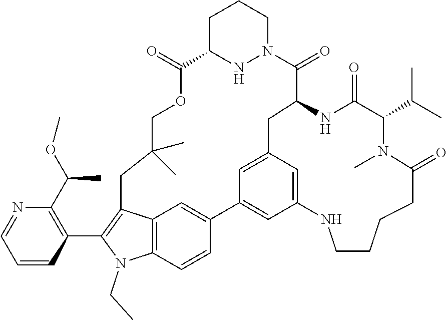 |
| A168 | 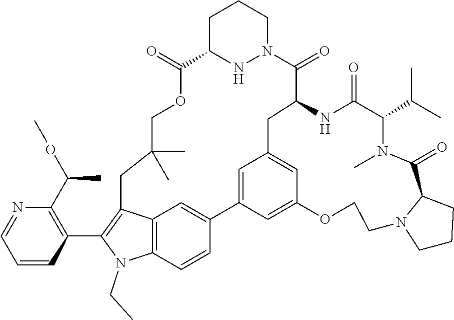 |
| A169 | 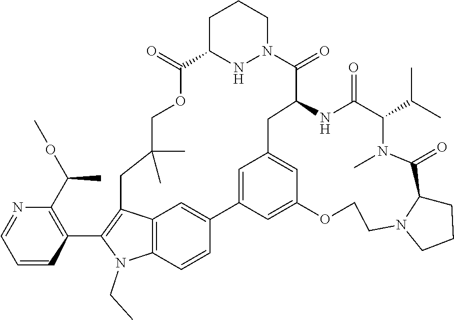 |
| A170 | 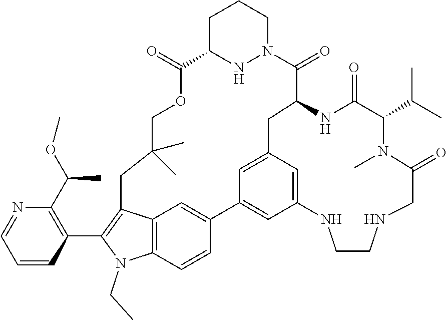 |
| A171 | 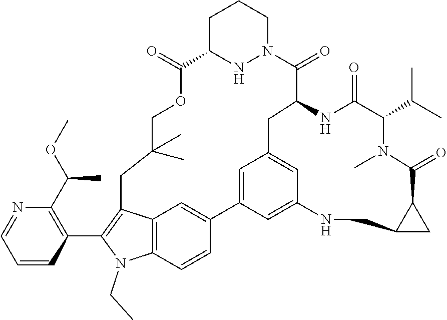 |
| A172 | 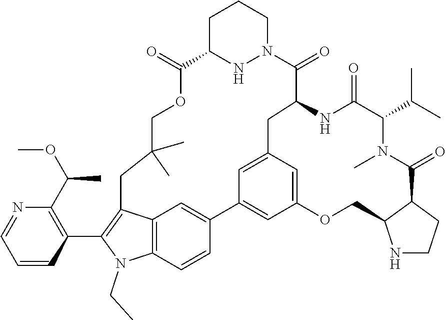 |
| A173 | 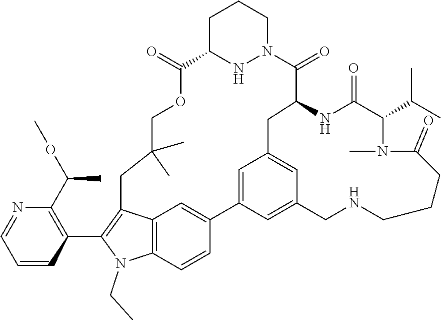 |
| A174 | 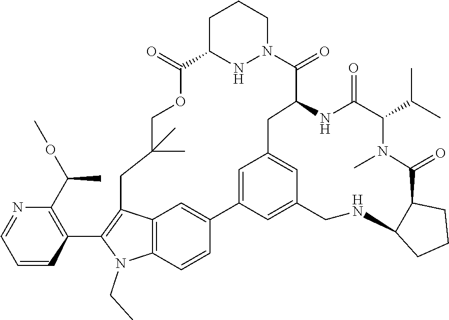 |
| A175 | 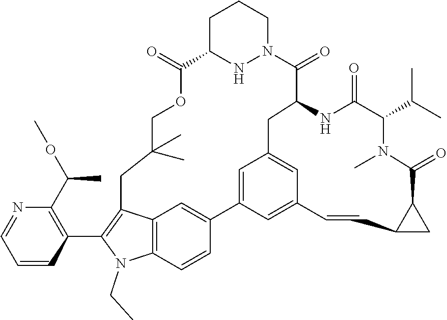 |
| A176 | 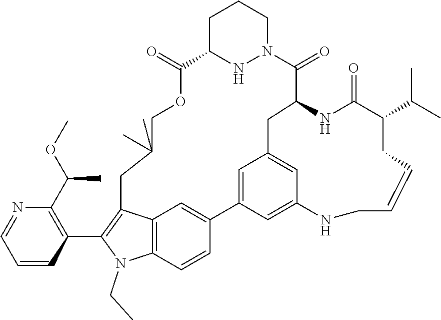 |
| A177 | 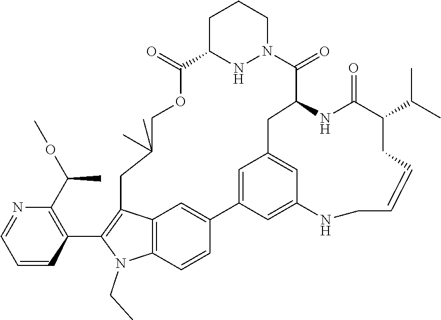 |
| A178 | 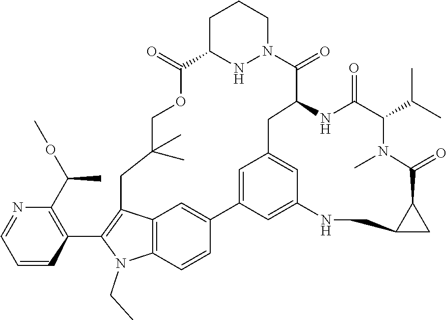 |
| A179 | 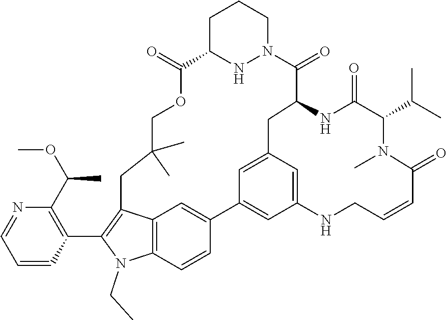 |
| A180 | 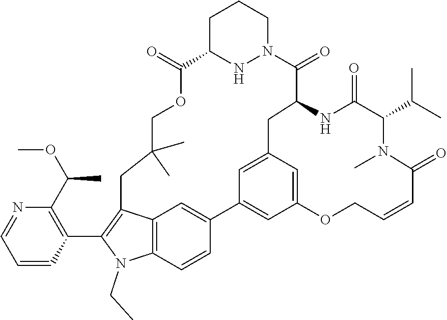 |
| A181 | 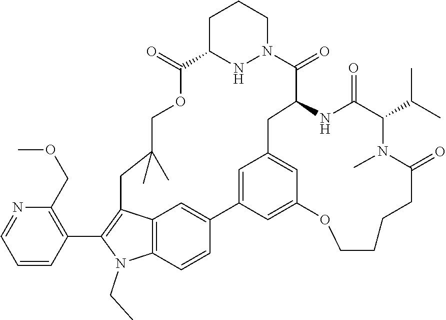 [SMILES: CCn1c(-c2cccnc2COC)c2c3cc(ccc31)-c1cc3cc(c1)OCCCCC(=O)N(C)[C@@H](C(C)C)C(=O)N[C@@H](C3)C(=O)N1CCC[C@H](N1)C(=O)OCC(C)(C)C2] |
| A182 | 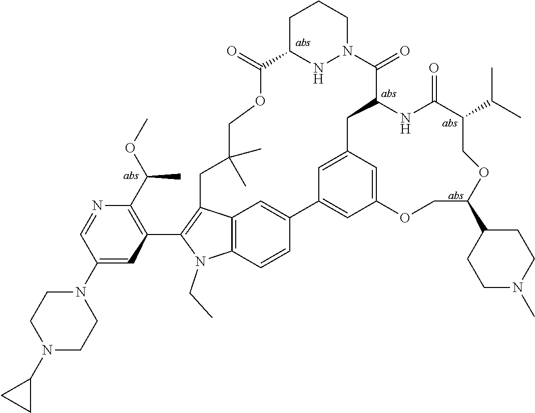 |
| A183 | 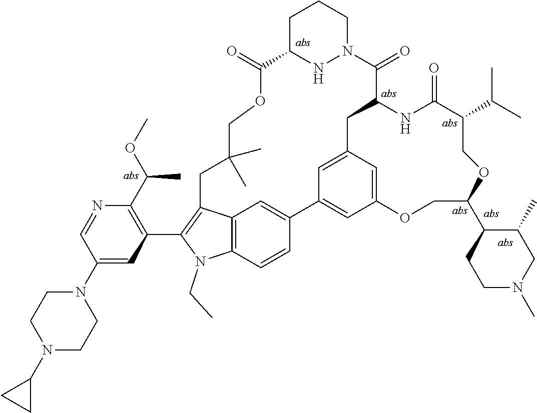 |
| A184 | 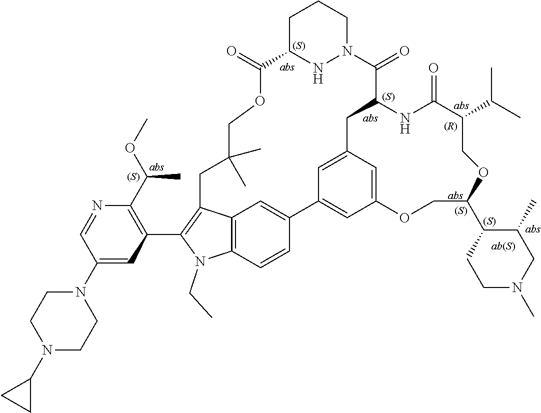 |
| A185 | 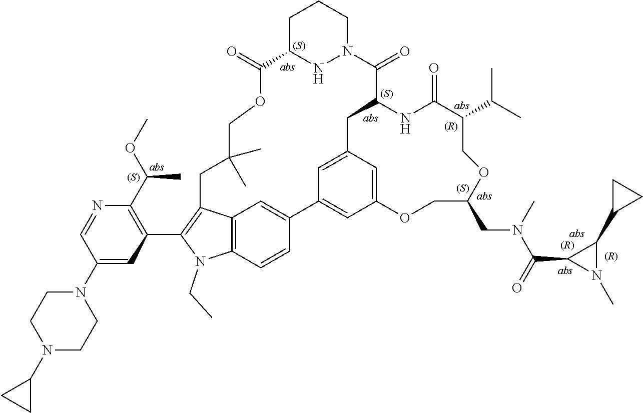 |
| A186 | 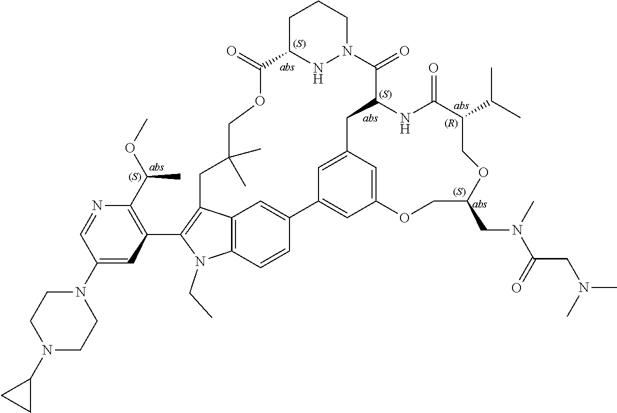 |
| A187 | 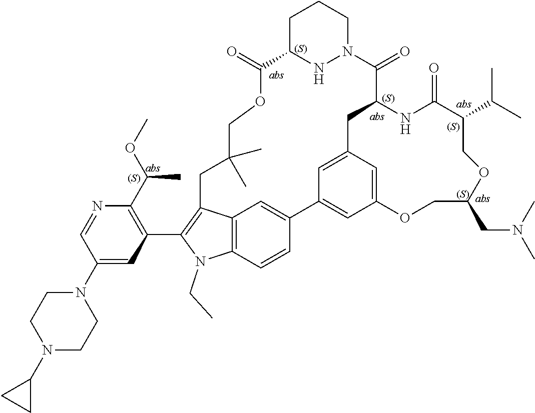 |
| A188 | 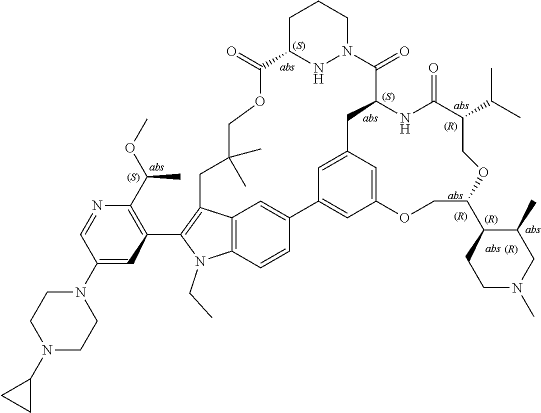 |
| A189 | 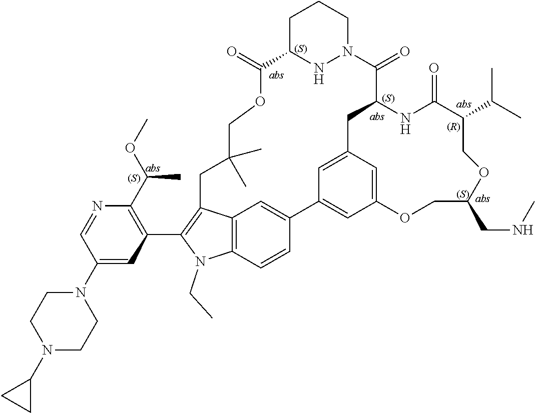 |
| A190 | 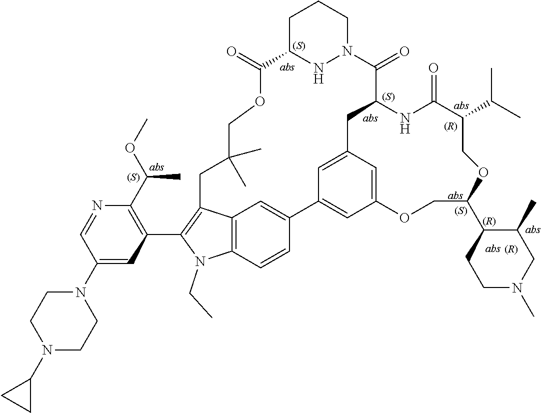 |
| A191 | 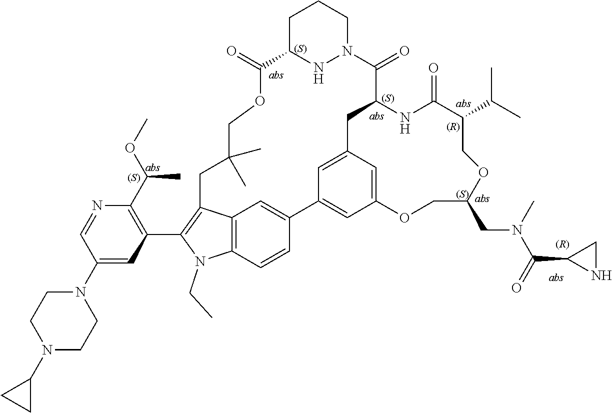 |
| A192 | 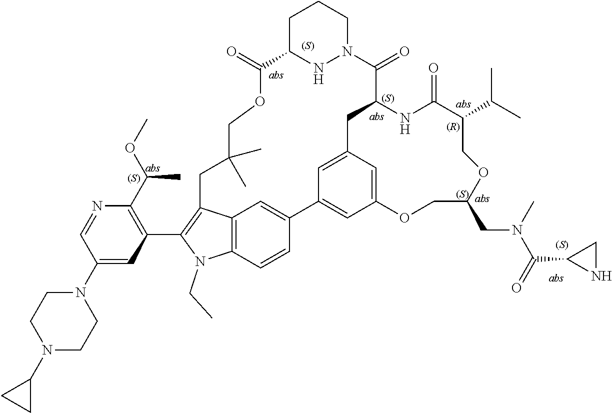 |
| A193 | 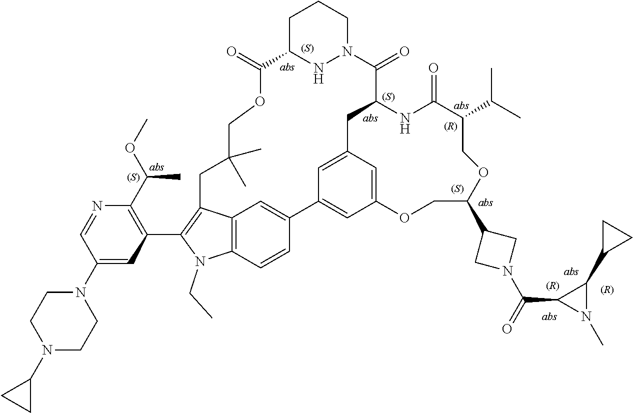 |
| A194 | 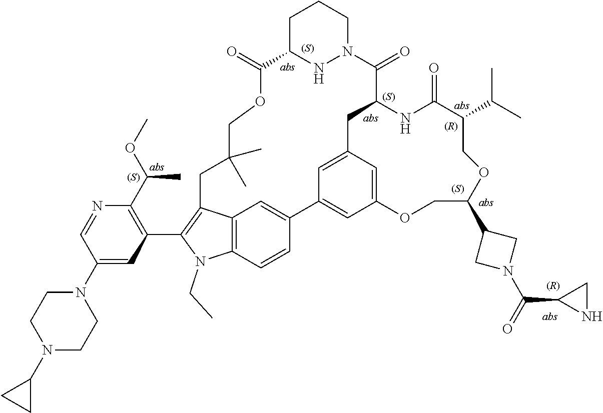 |
| A195 | 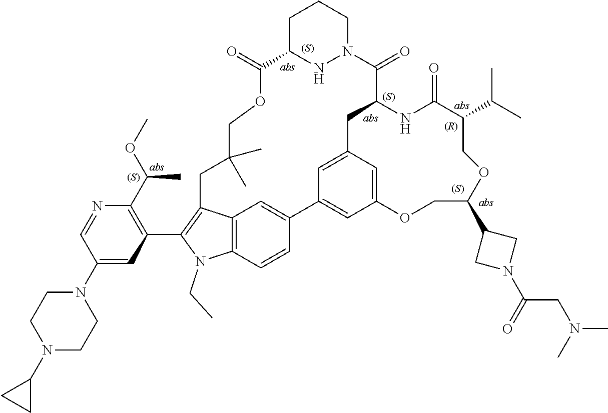 |
| A196 | 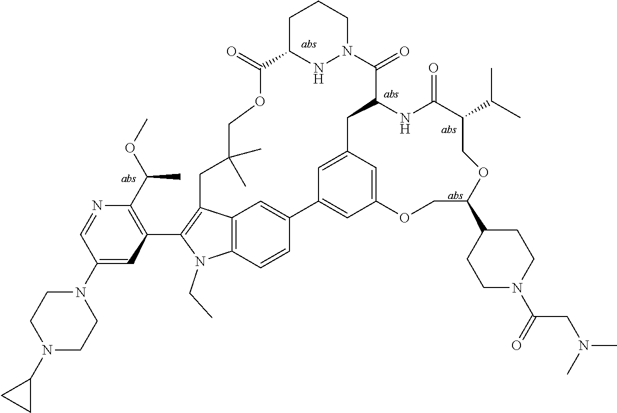 |
| A197 | 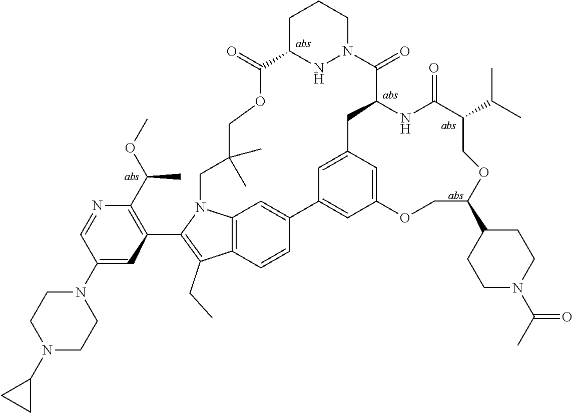 |
| A198 | 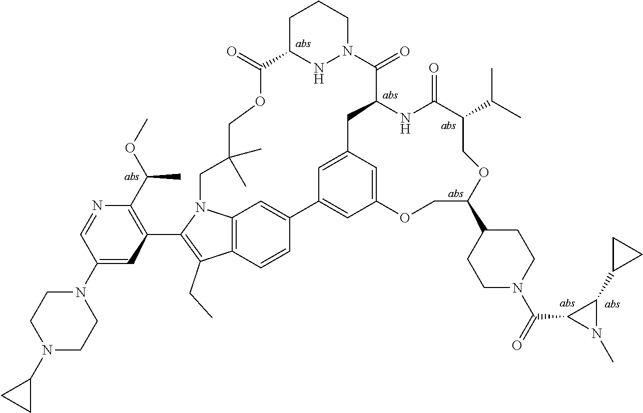 |
| A199 | 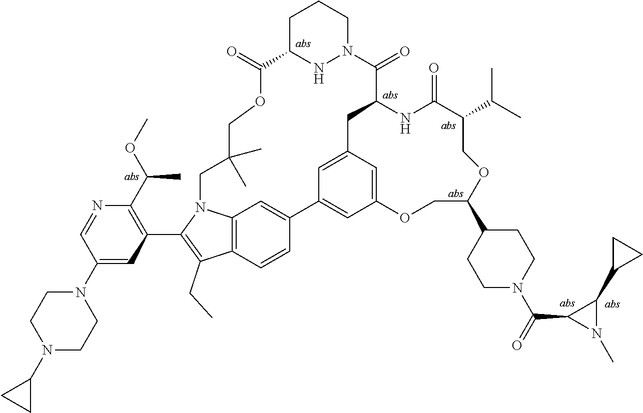 |
| A200 | 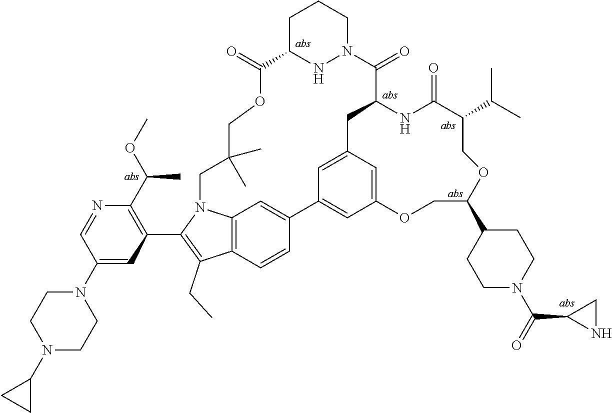 |
| A201 | 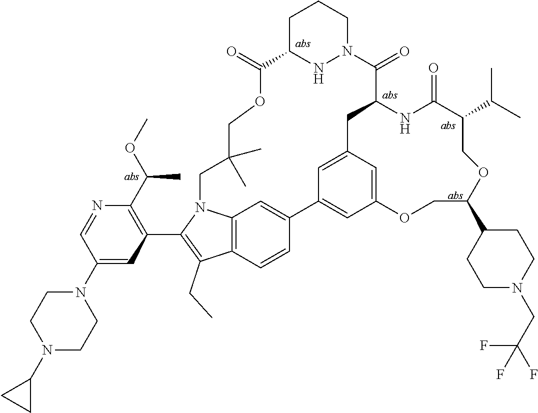 |
| A202 | 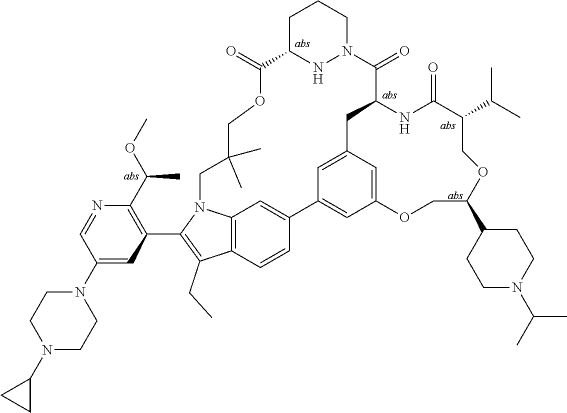 |
| A203 | 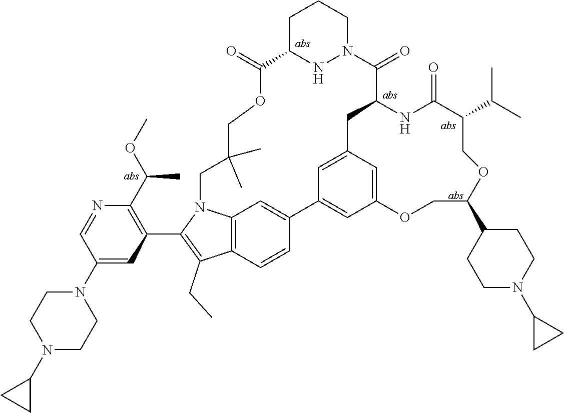 |
| A204 | 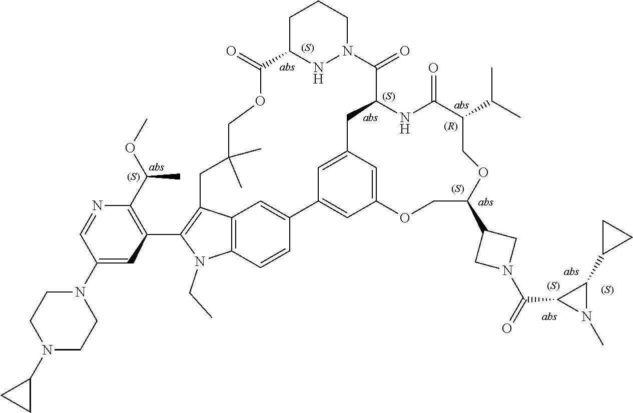 |
| A205 | 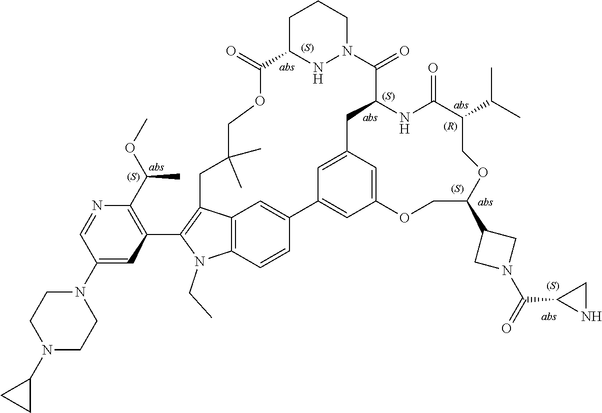 |
| A206 | 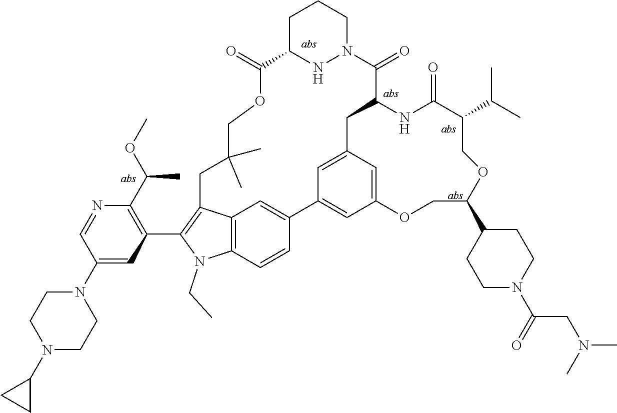 |
| A207 | 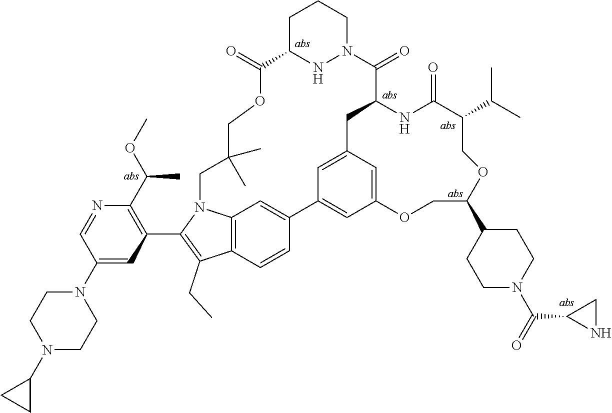 |
| A208 | 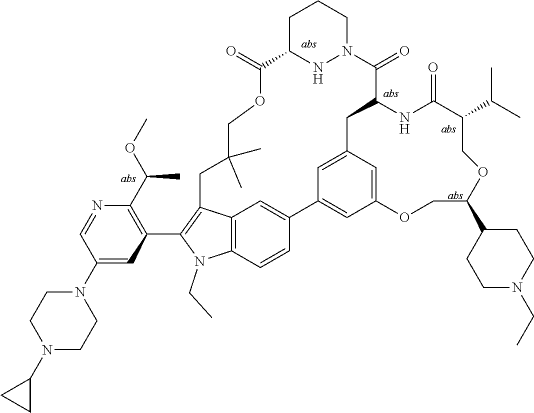 |
| A209 | 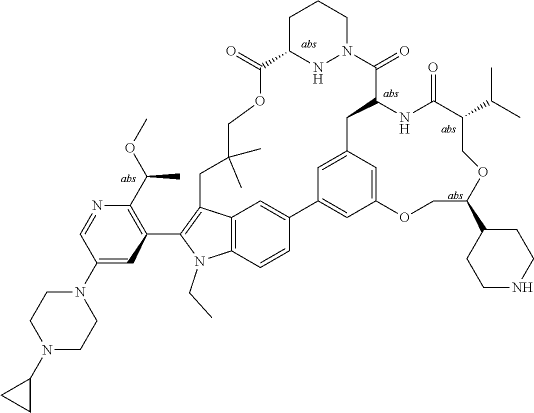 |
| A210 | 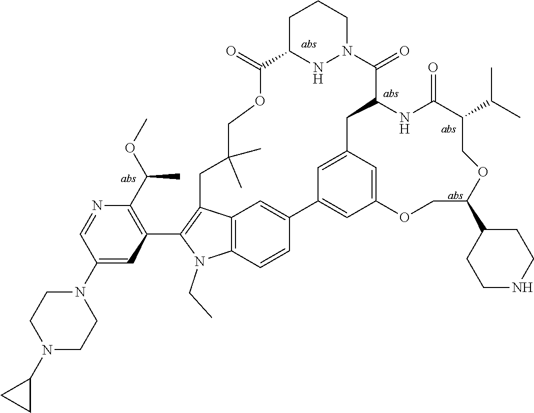 |
| A211 | 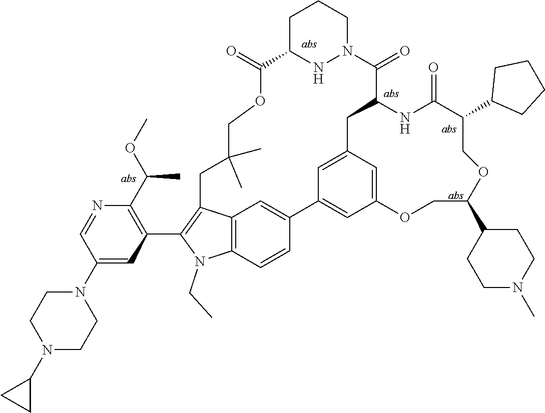 |
| A212 | 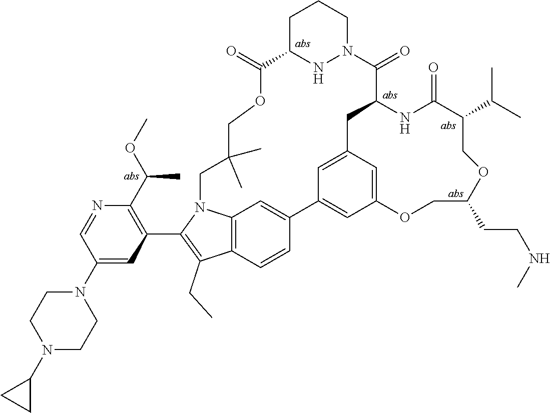 |
| A213 | 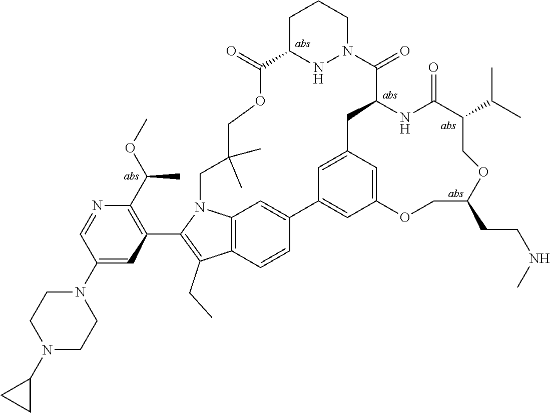 |
| A214 | 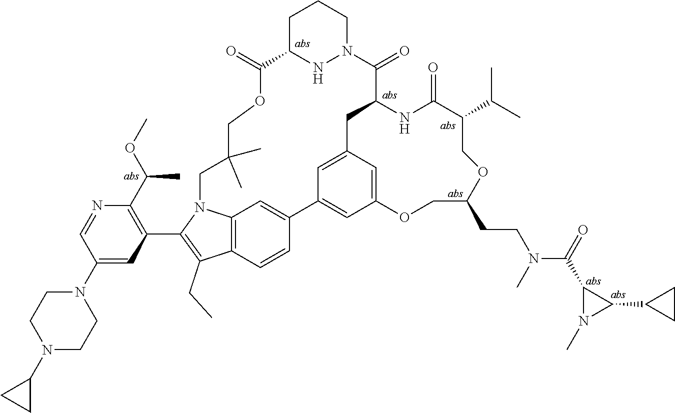 |
| A215 | 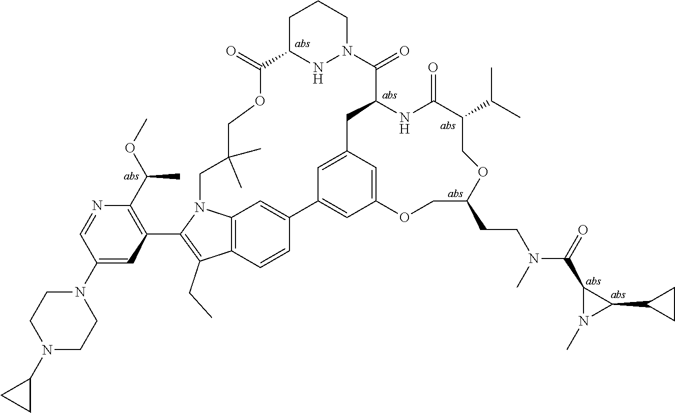 |
| A216 | 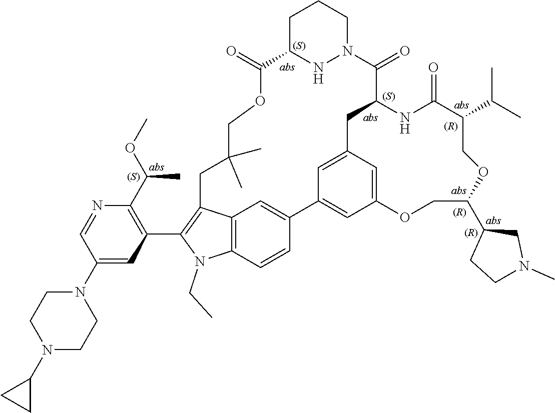 |
| A217 | 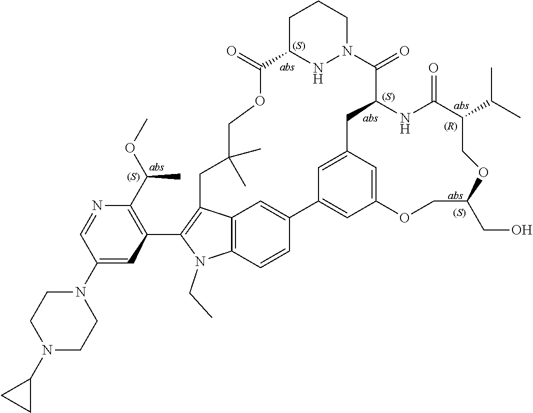 |
| A218 | 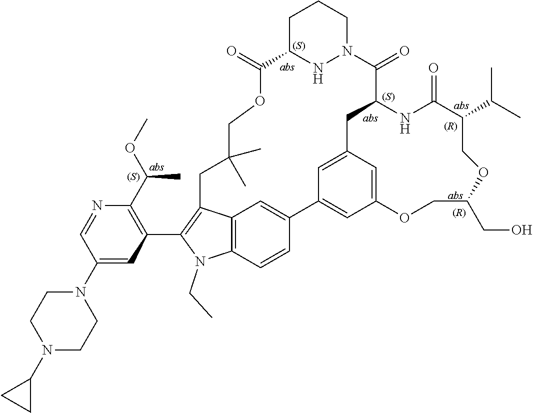 |
| A219 | 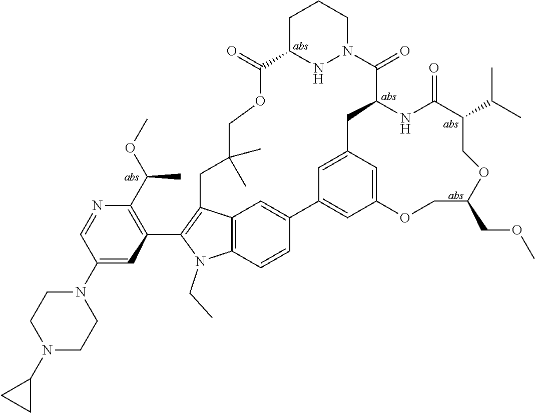 |
| A220 | 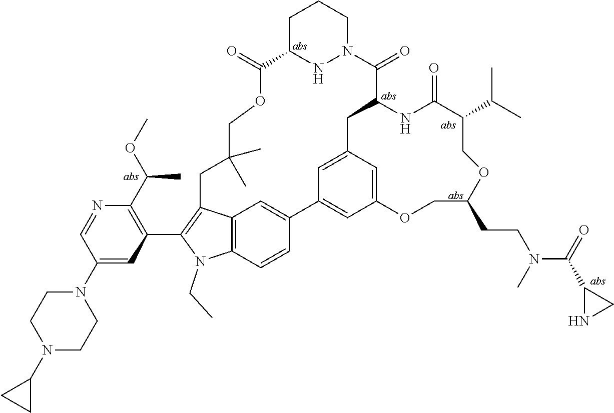 |
| A221 | 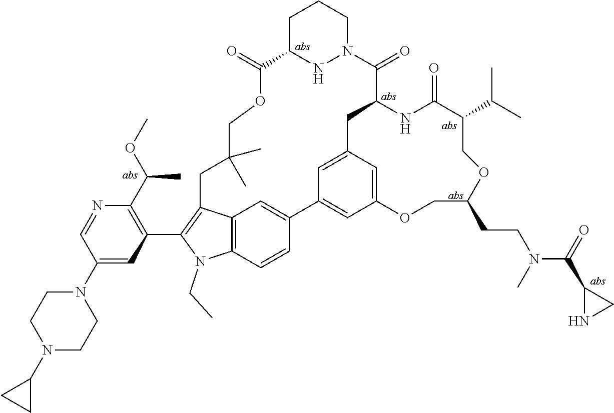 |
| A222 | 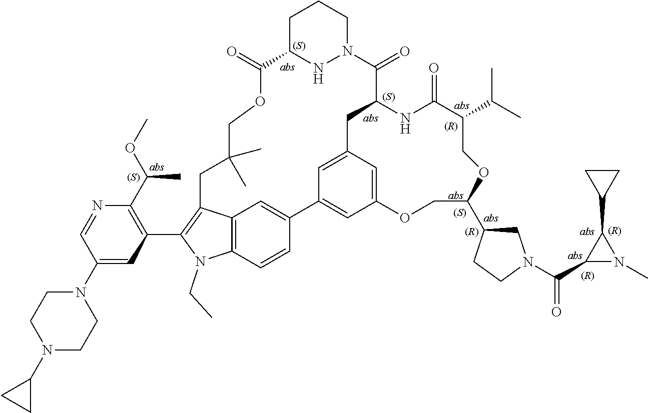 |
| A223 | 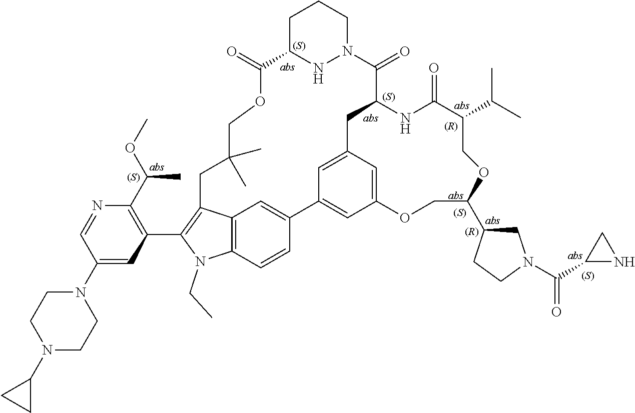 |
| A224 | 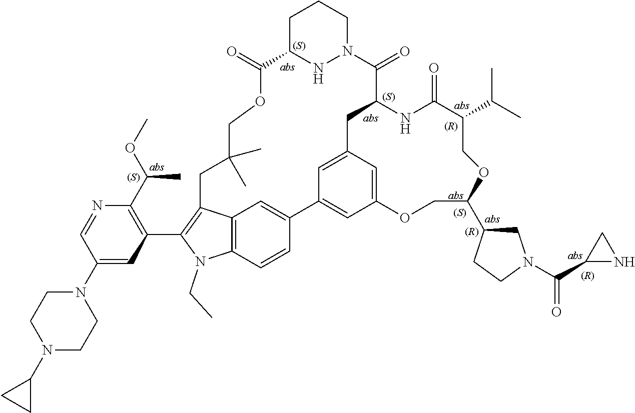 |
| A225 | 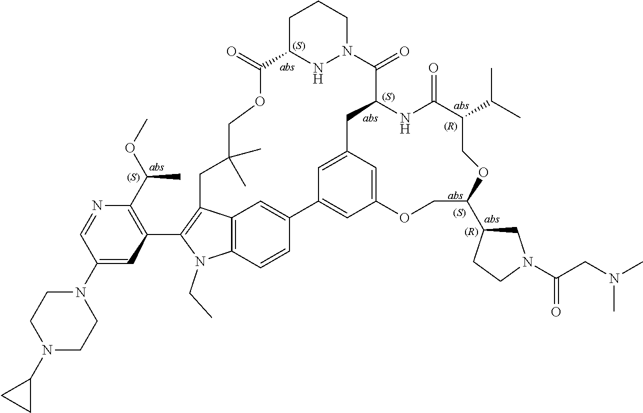 |
| A226 | 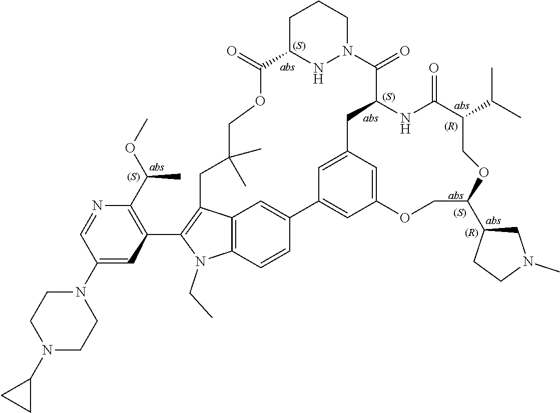 |
| A227 | 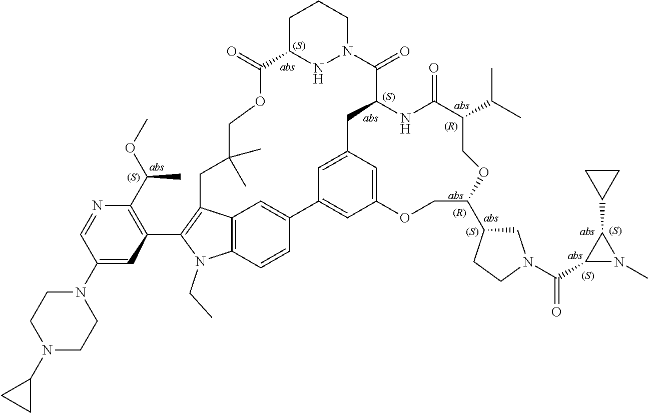 |
| A228 | 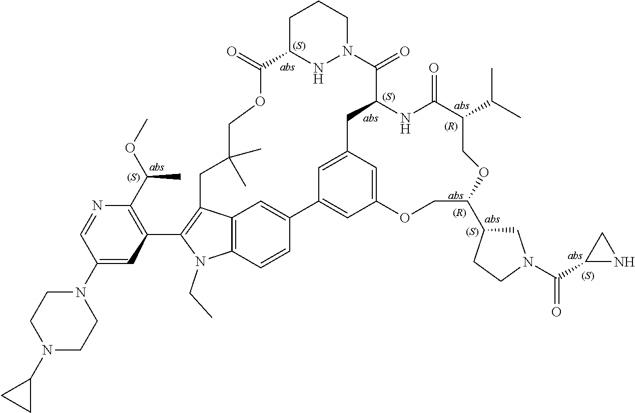 |
| A229 | 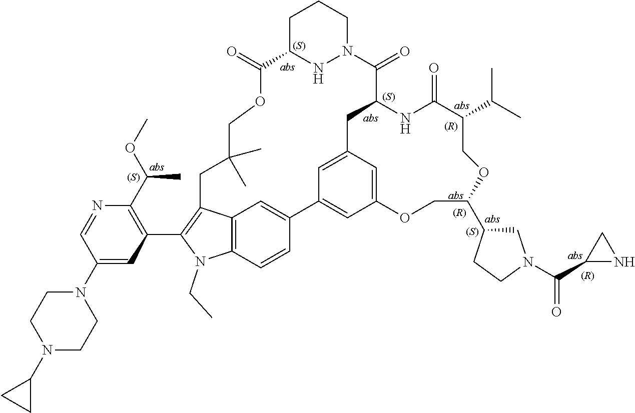 |
| A230 | 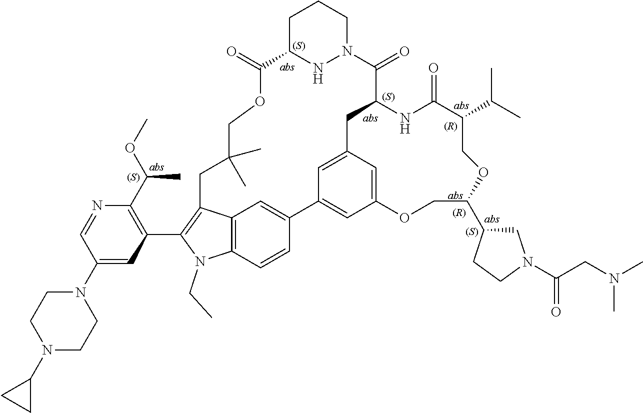 |
| A231 | 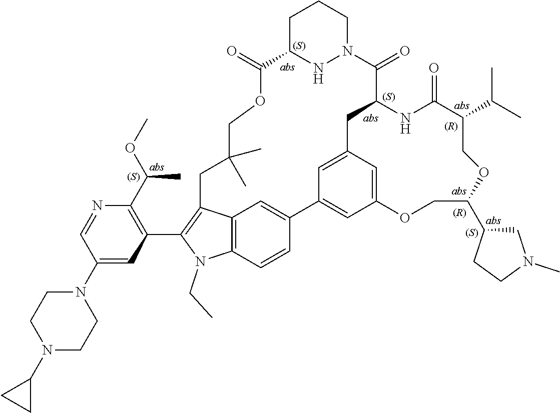 |
| A232 | 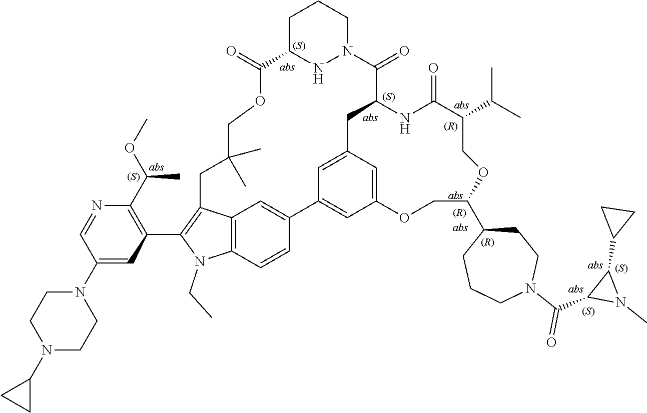 |
| A233 | 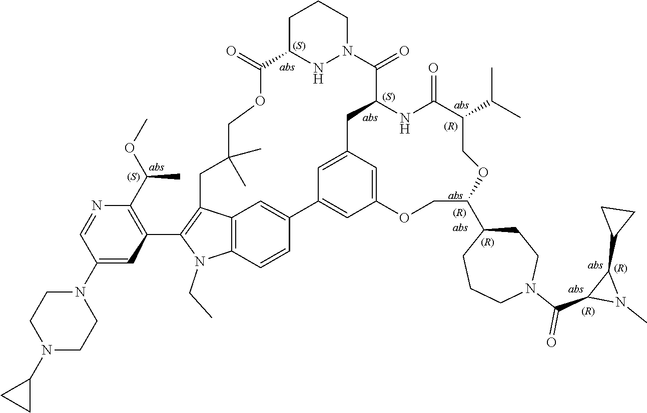 |
| A234 | 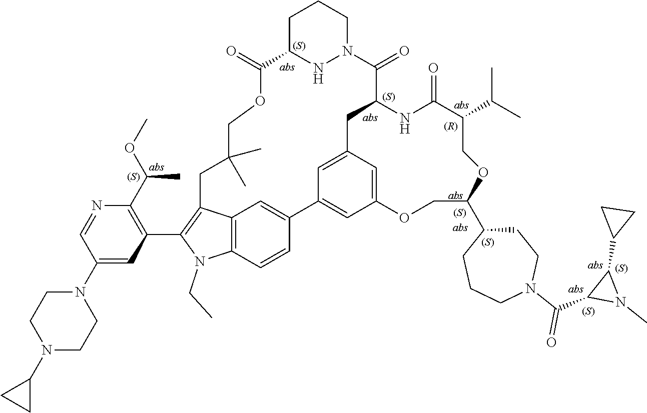 |
| A235 | 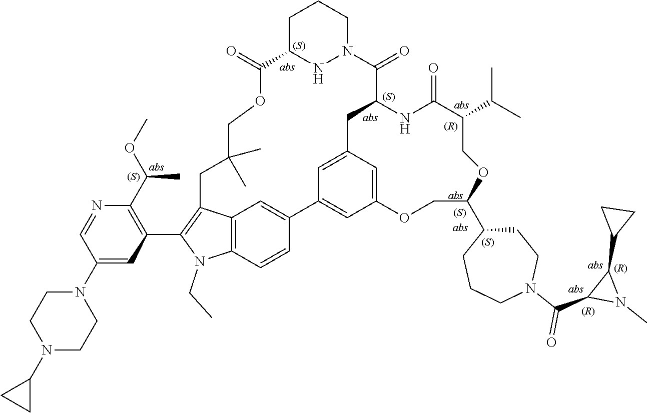 |
| A236 | 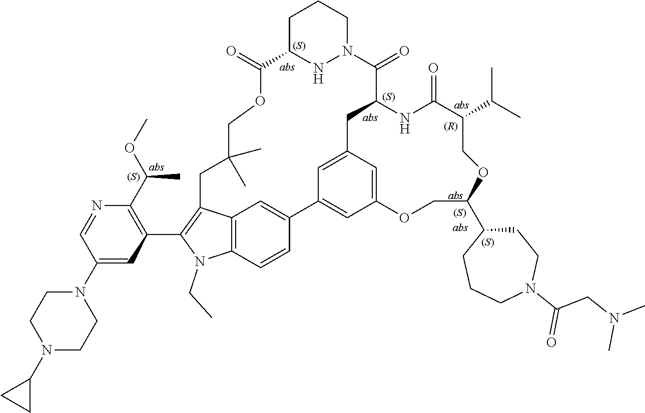 |
| A237 | 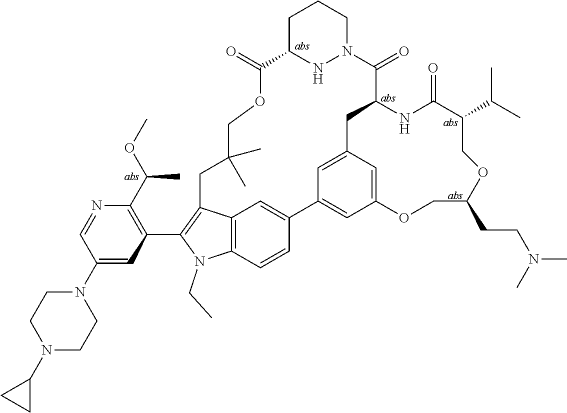 |
| A238 | 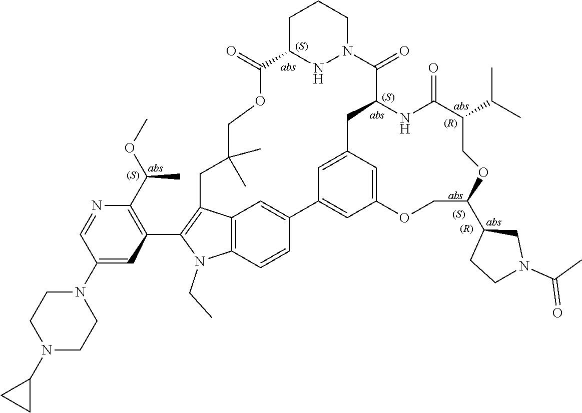 |
| A239 | 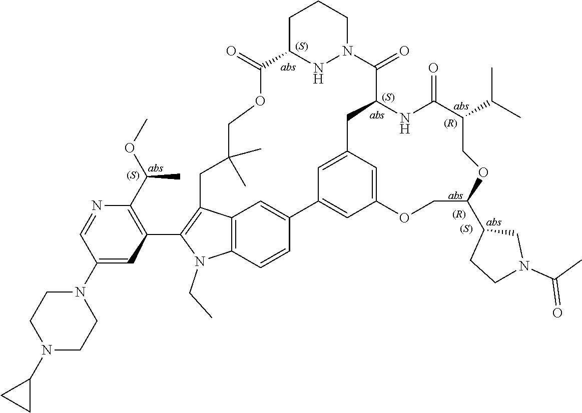 |
| A240 | 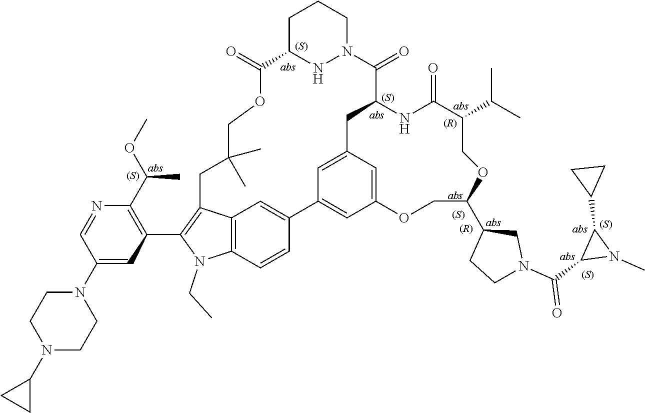 |
| A241 | 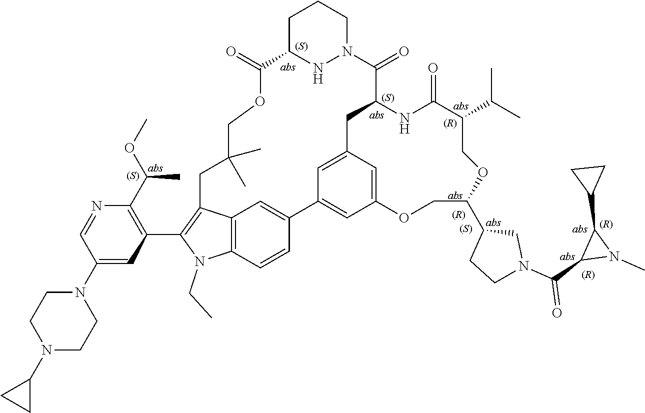 |
| A242 | 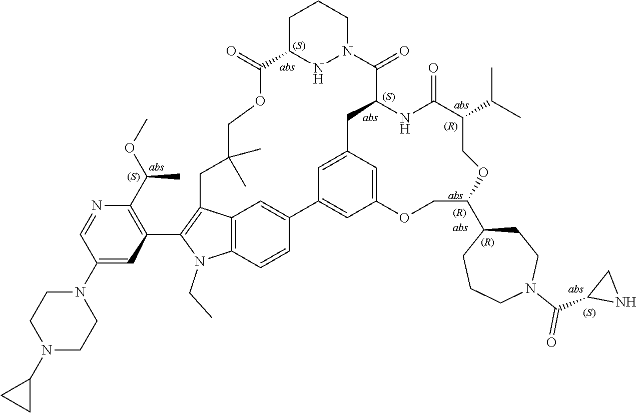 |
| A243 | 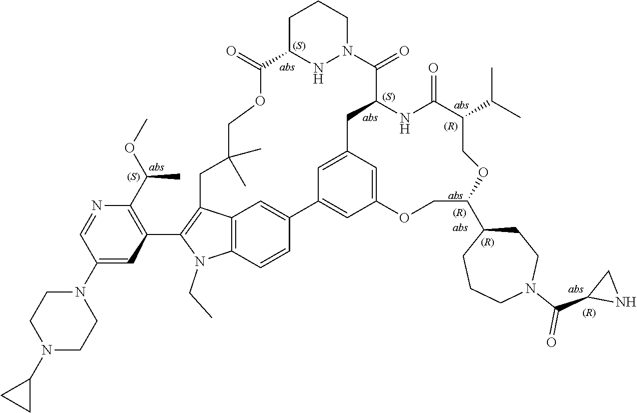 |
| A244 | 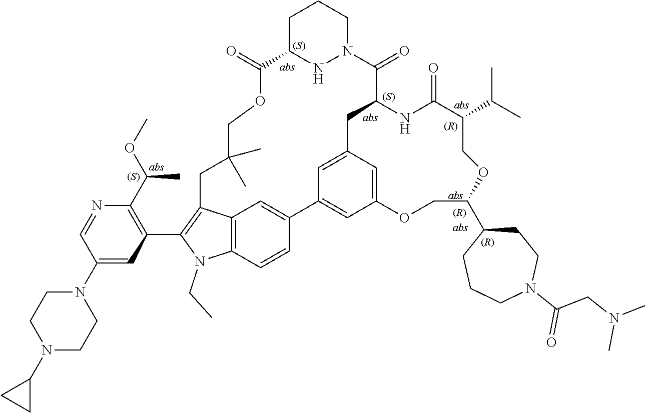 |
| A245 | 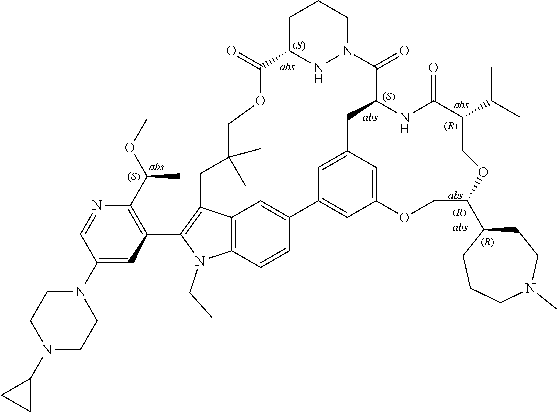 |
| A246 | 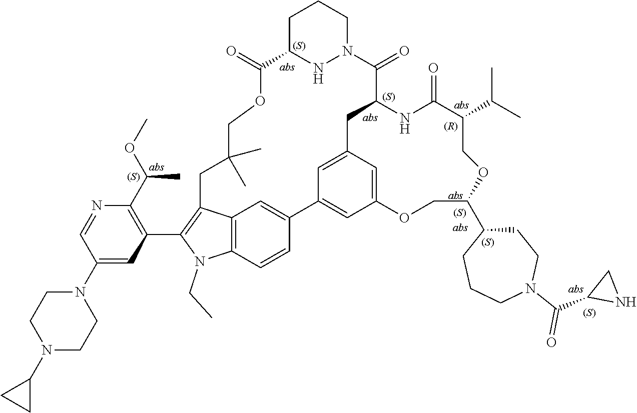 |
| A247 | 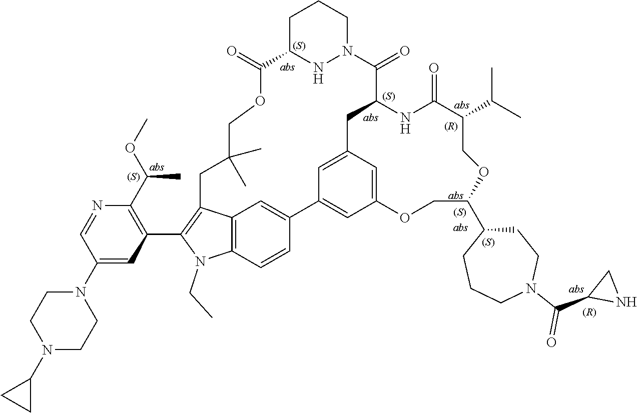 |
| A248 | 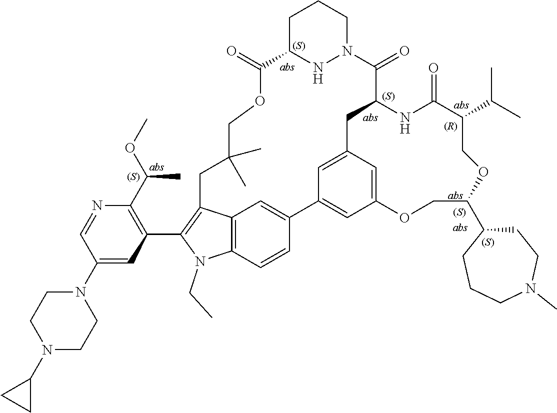 |
| A249 | 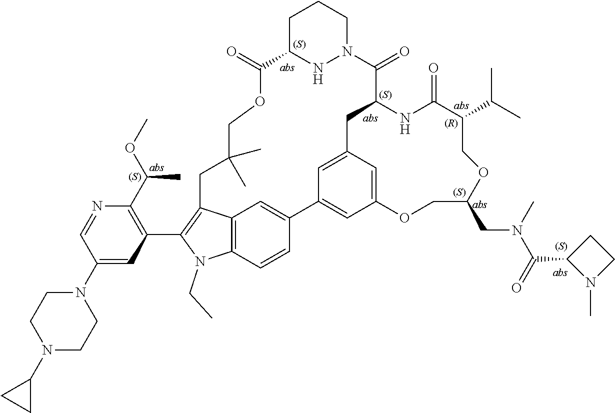 |
| A250 | 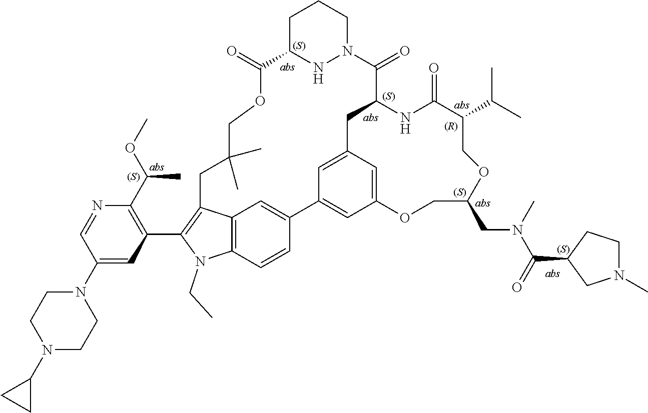 |
| A251 | 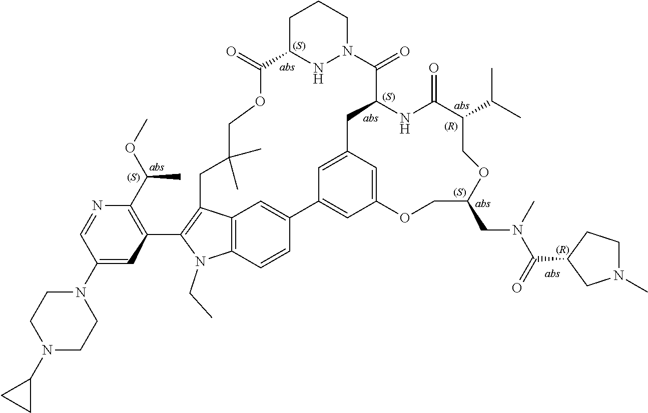 |
| A252 | 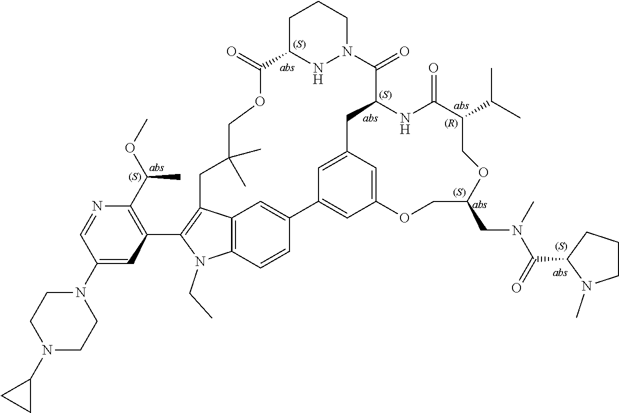 |
| A253 | 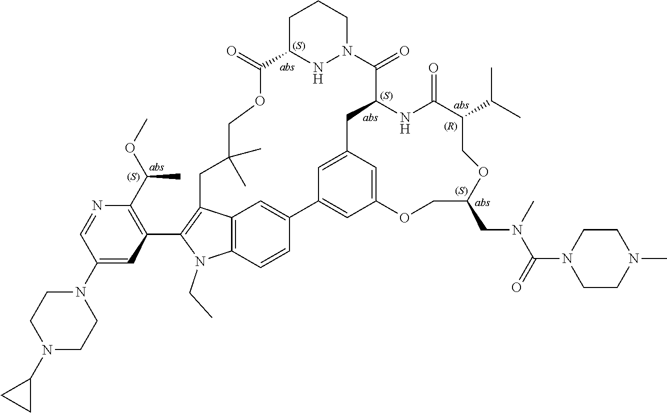 |
| A254 | 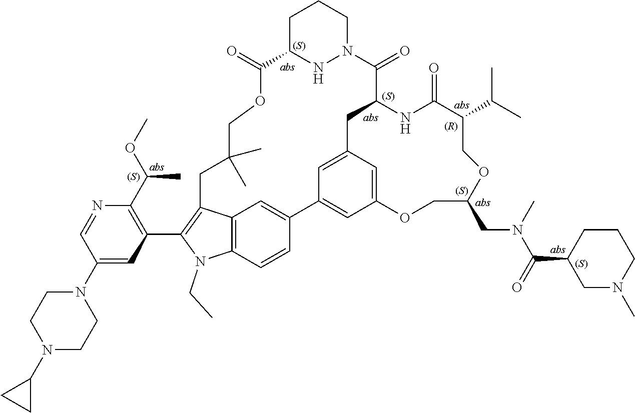 |
| A255 | 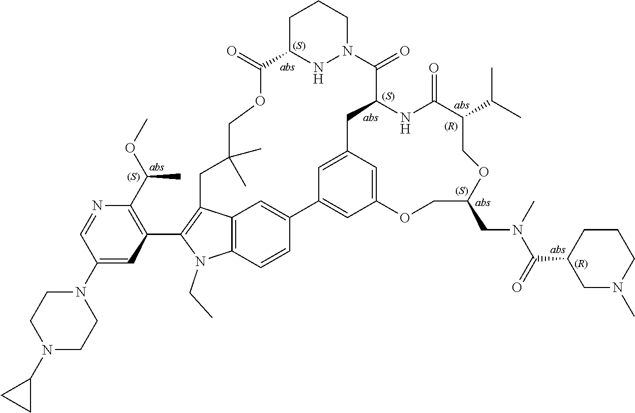 |
| A256 | 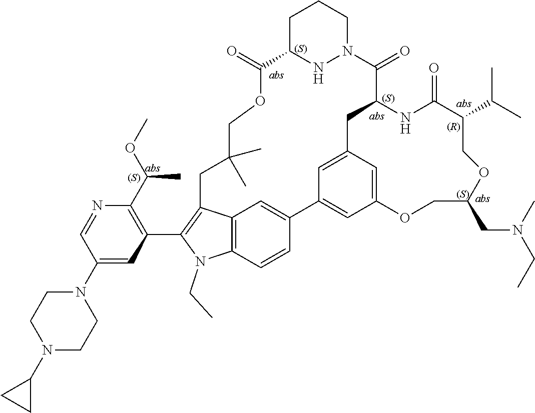 |
| A257 | 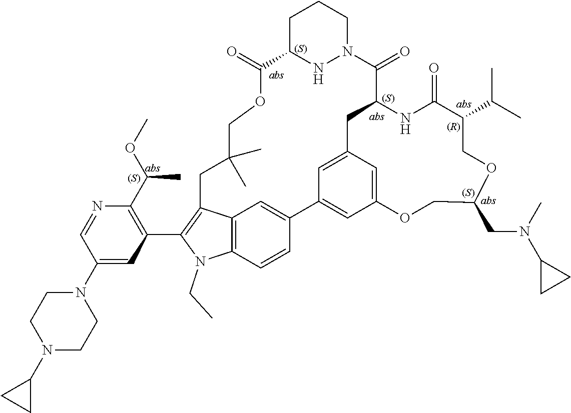 |
| A258 | 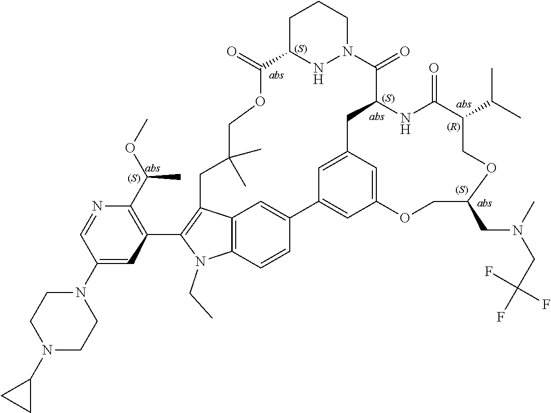 |
| A259 | 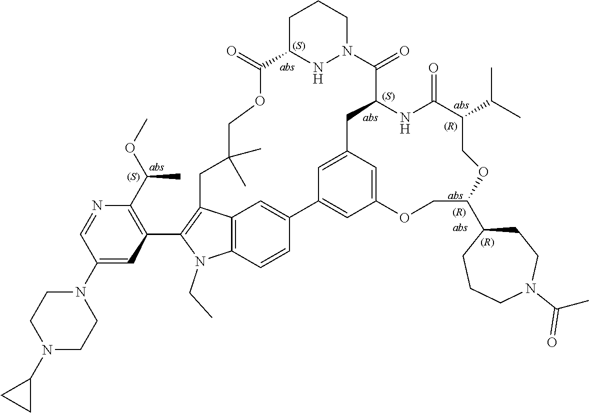 |
| A260 | 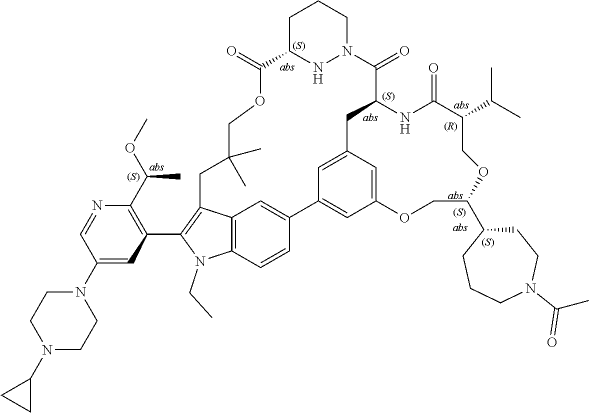 |
| A261 | 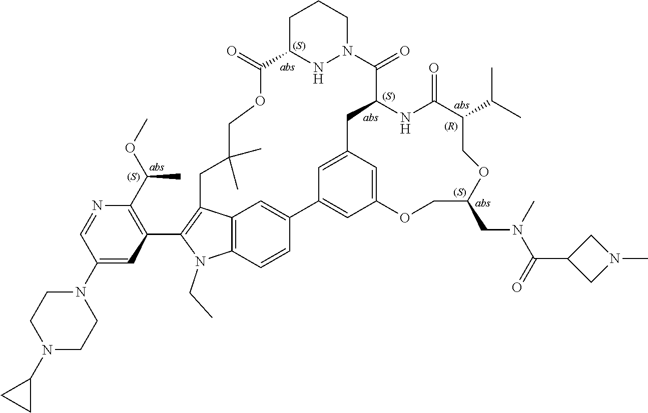 |
| A262 | 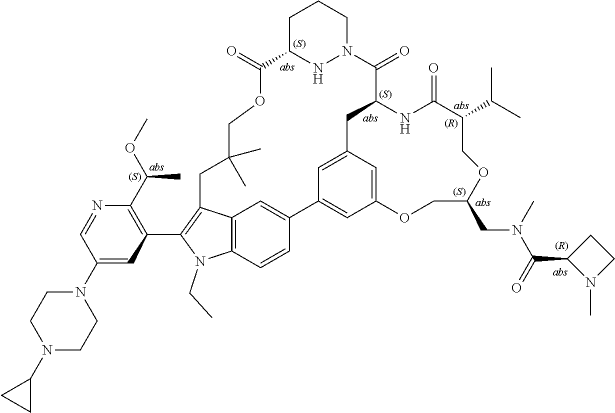 |
| A263 | 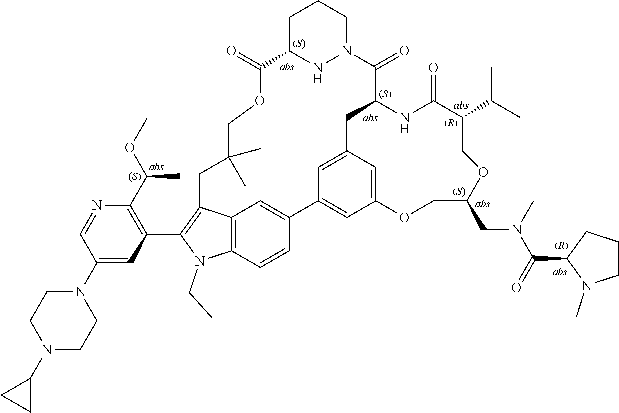 |
| A264 | 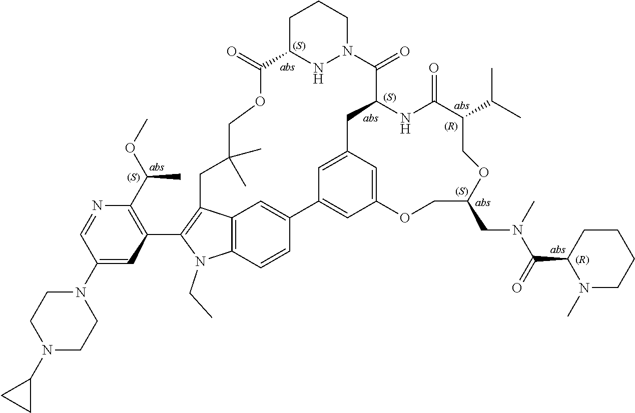 |
| A265 | 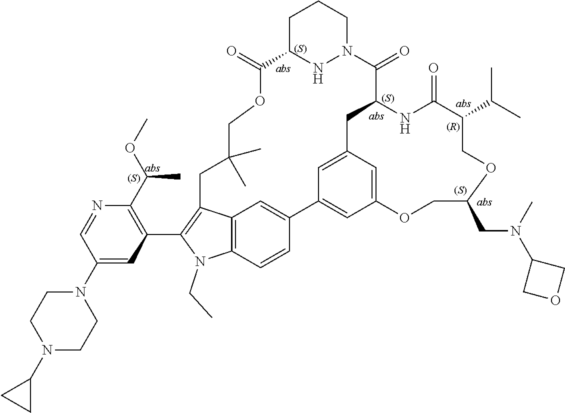 |
| A266 | 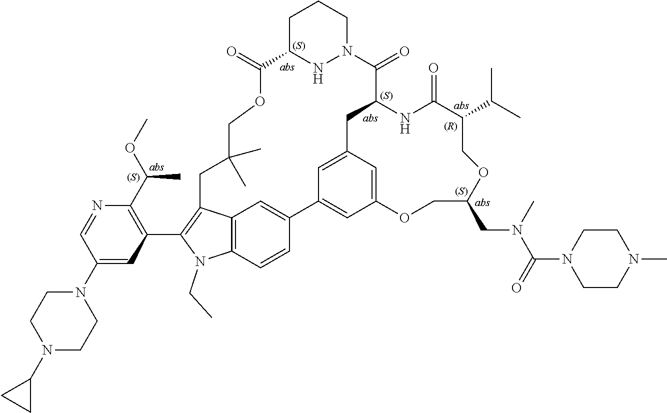 |
| A267 | 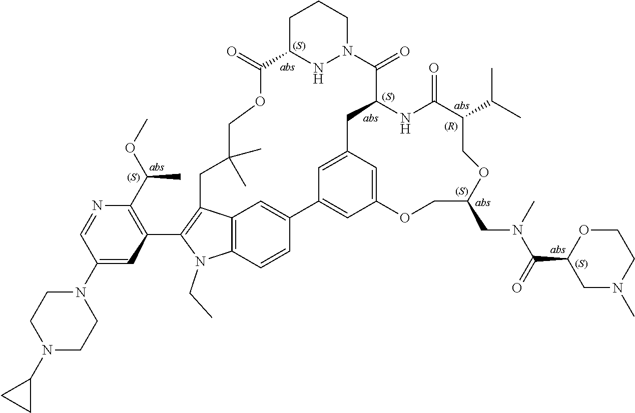 |
| A268 | 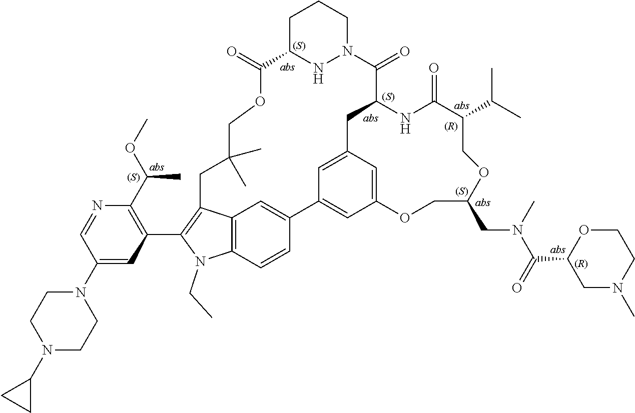 |
| A269 | 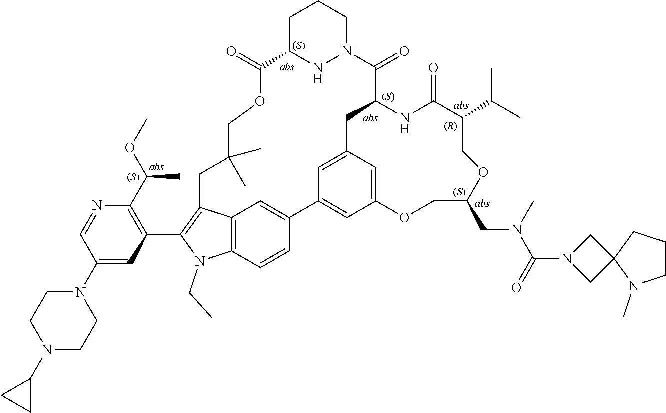 |
| A270 | 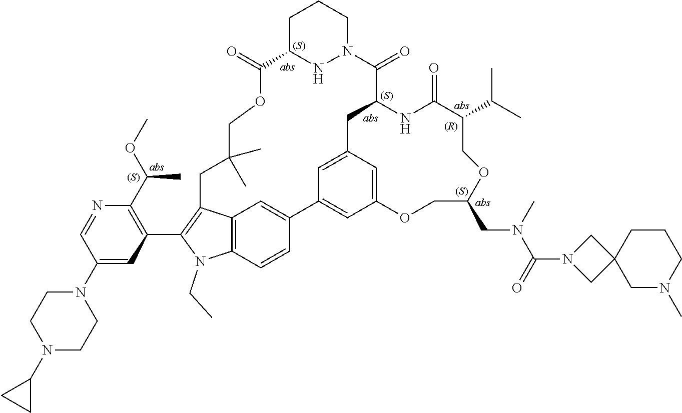 |
| A271 | 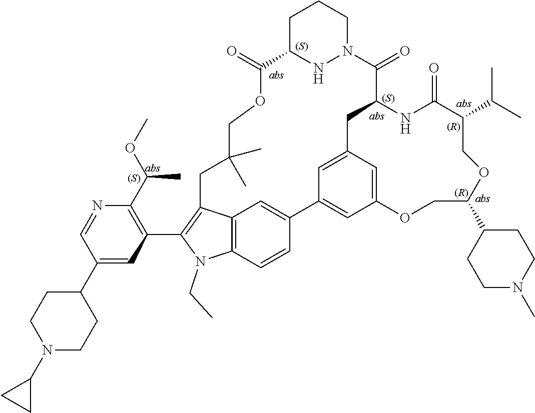 |
| A272 | 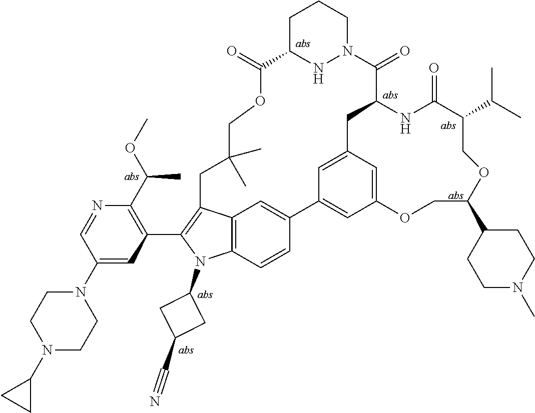 |
| A273 | 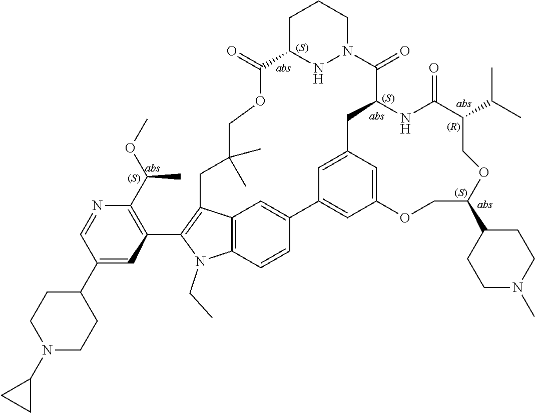 |
| A274 | 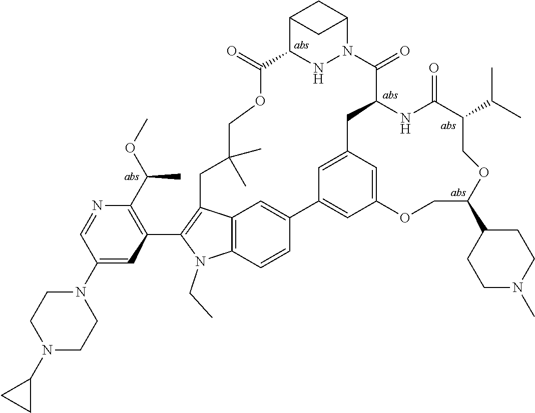 |
| A275 | 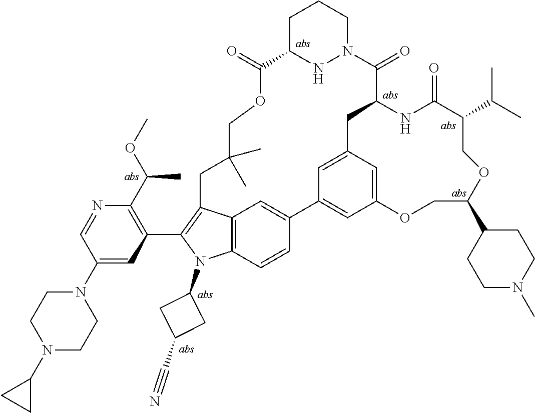 |
| A276 | 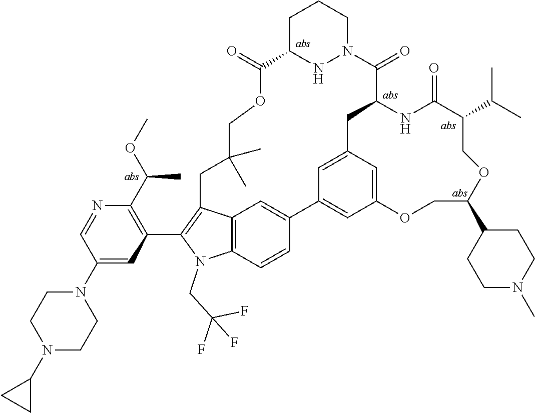 |
| A277 | 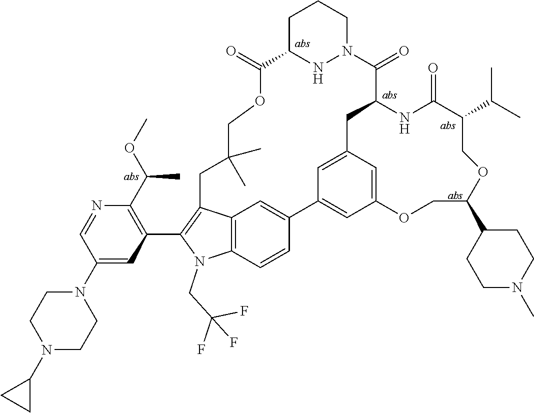 |
| A278 | 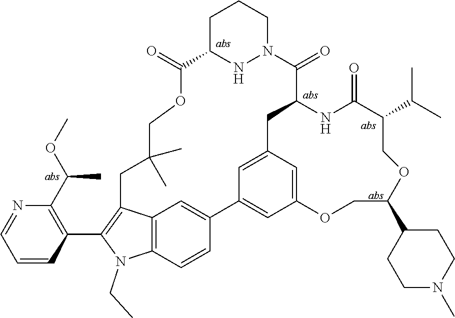 |
| A279 | 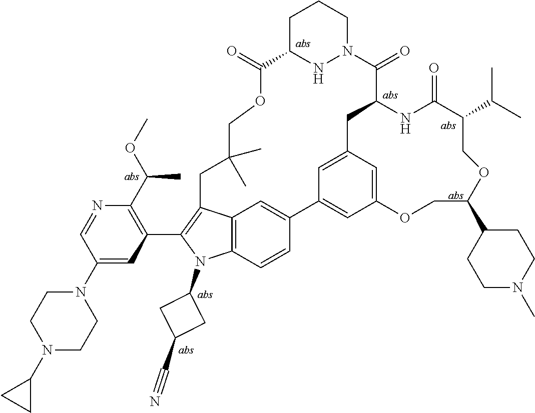 |
| A280 | 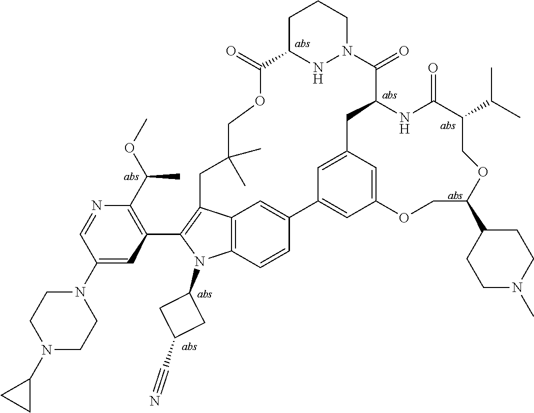 |
| A281 | 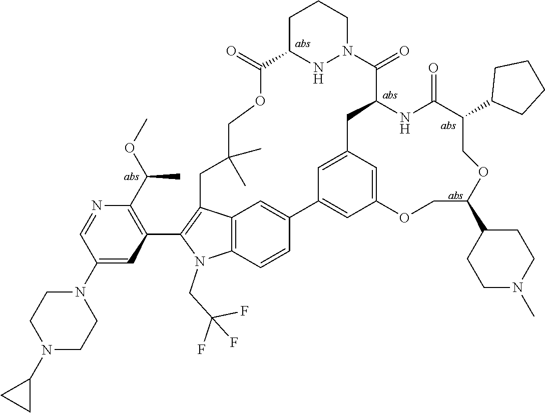 |
| A282 | 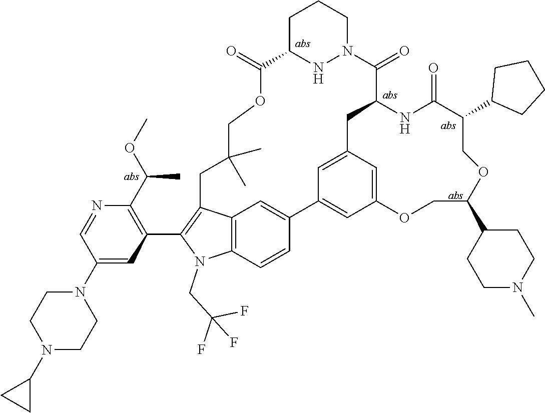 |
| A283 | 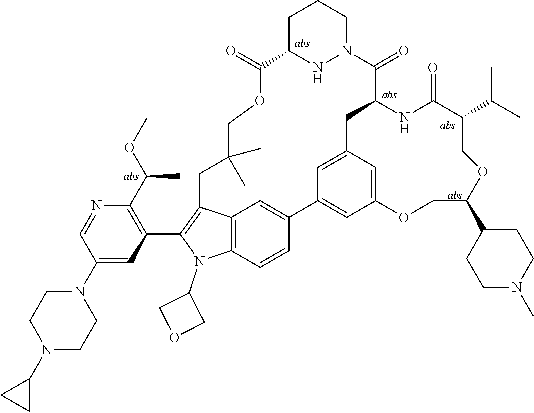 |
| A284 | 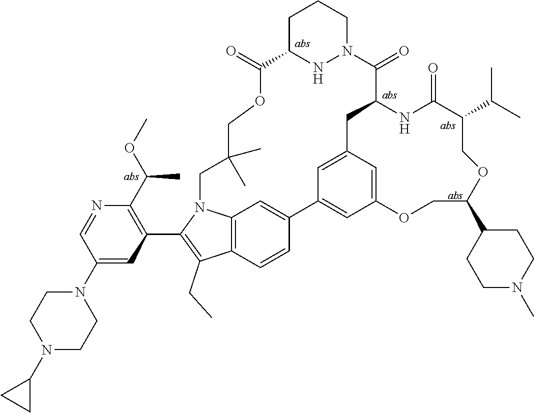 |
| A285 | 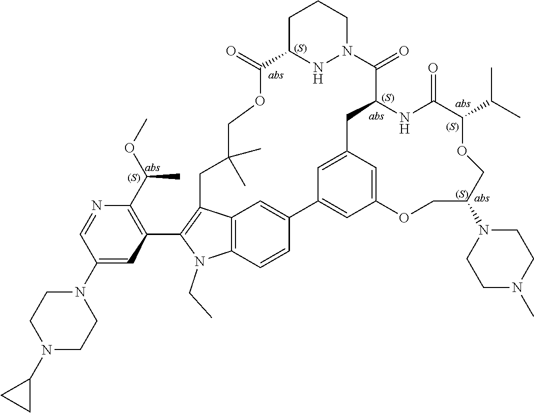 |
| A286 | 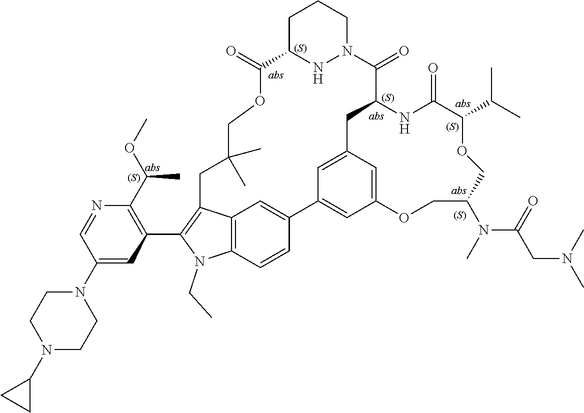 |
| A287 | 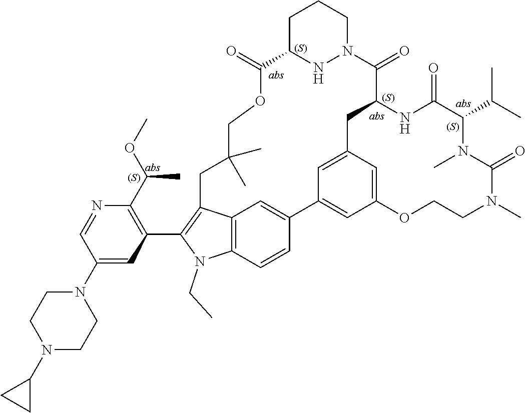 |
| A288 | 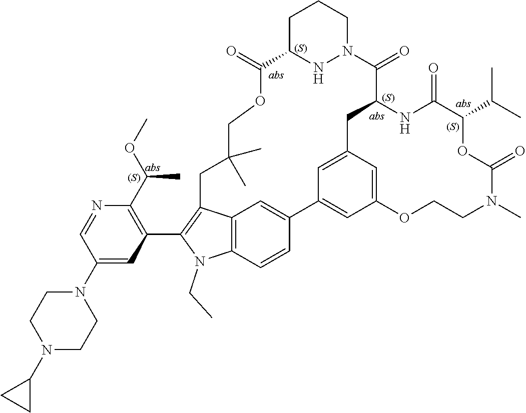 |
| A289 | 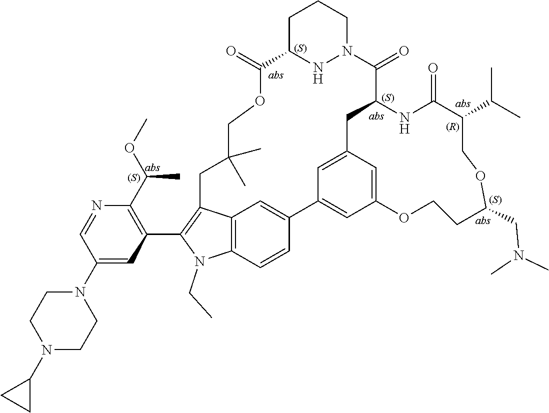 |
| A290 | 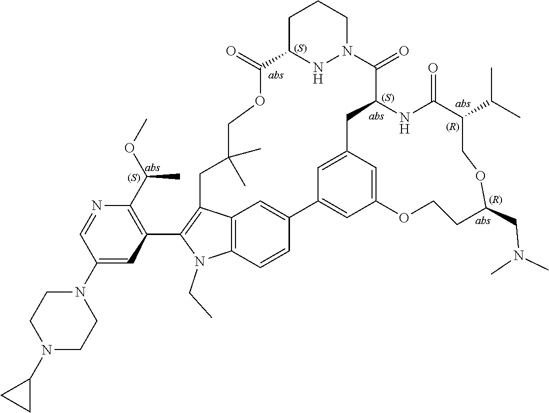 |
| A291 | 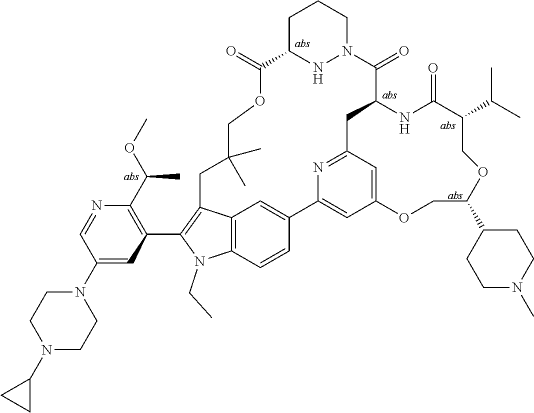 |

[symbol]

indicates data missing or illegible when filed

Also provided is a pharmaceutical composition comprising a compound of the present invention, or a pharmaceutically acceptable salt thereof, and a pharmaceutically acceptable excipient.

Further provided is a conjugate, or salt thereof, of a compound of the present invention, wherein the compound of the present invention has a covalent warhead, bound to a monovalent organic moiety.

In some embodiments of conjugates of the present invention, the monovalent organic moiety is a protein. In some embodiments, the protein is a Ras protein. In some embodiments, the Ras protein is K-Ras G12C, K-Ras G13C, H-Ras G12C, H-Ras G13C, N-Ras G12C, N-Ras G13C, K-Ras Q61H, H-Ras Q61H, N-Ras Q61H, N-Ras Q61K or N-Ras Q61R.

Compounds of the present invention are also adaptable for uses in antibody-drug conjugates as well as degrader applications.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer is pancreatic cancer, colorectal cancer, non-small cell lung cancer, acute myeloid leukemia, or multiple myeloma. In some embodiments, the cancer comprises a Ras mutation, such as K-Ras Q61H, H-Ras Q61H, or N-Ras Q61H. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras Q61H, H-Ras Q61H, or N-Ras Q61H. Other Ras proteins are described herein. The cell may be a cancer cell, such as a pancreatic cancer cell, a colorectal cancer cell, a non-small cell lung cancer cell, an acute myeloid leukemia cell, a multiple myeloma cell, a thyroid gland adenocarcinoma cell, a myelodysplastic syndrome cell, or a squamous cell lung carcinoma cell. In some embodiments, the cell is a pancreatic cancer cell, a colorectal cancer cell, a non-small cell lung cancer cell, an acute myeloid leukemia cell, or a multiple myeloma cell, Other cancer types are described herein. The cell may be in vivo or in vitro.

With respect to compounds of the present invention, one stereoisomer may exhibit better inhibition than another stereoisomer. For example, one atropisomer may exhibit inhibition, whereas the other atropisomer may exhibit little or no inhibition.

In some embodiments, a method or use described herein further comprises administering an additional anti-cancer therapy. In some embodiments, the additional anti-cancer therapy is a HER2 inhibitor, an EGFR inhibitor, a second Ras inhibitor, a SHP2 inhibitor, an SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, or a combination thereof. In some embodiments, the additional anticancer therapy is a SHP2 inhibitor. Other additional anti-cancer therapies are described herein.

### Methods of Synthesis

The compounds described herein may be made from commercially available starting materials or synthesized using known organic, inorganic, or enzymatic processes.

The compounds of the present invention (see, e.g., compounds of Table 1) can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. As a further example, synthetic methods described in WO 2020/132597, WO 2021/091982, WO 2021/091967, WO 2021/091956, WO 2022/060836, WO 2022/235864, WO 2022/235870, WO 2023/060253, and WO 2023/133543, the disclosure of each of which is incorporated herein by reference, may be useful in preparing compounds of the invention. These methods include but are not limited to those methods described in the Schemes below.

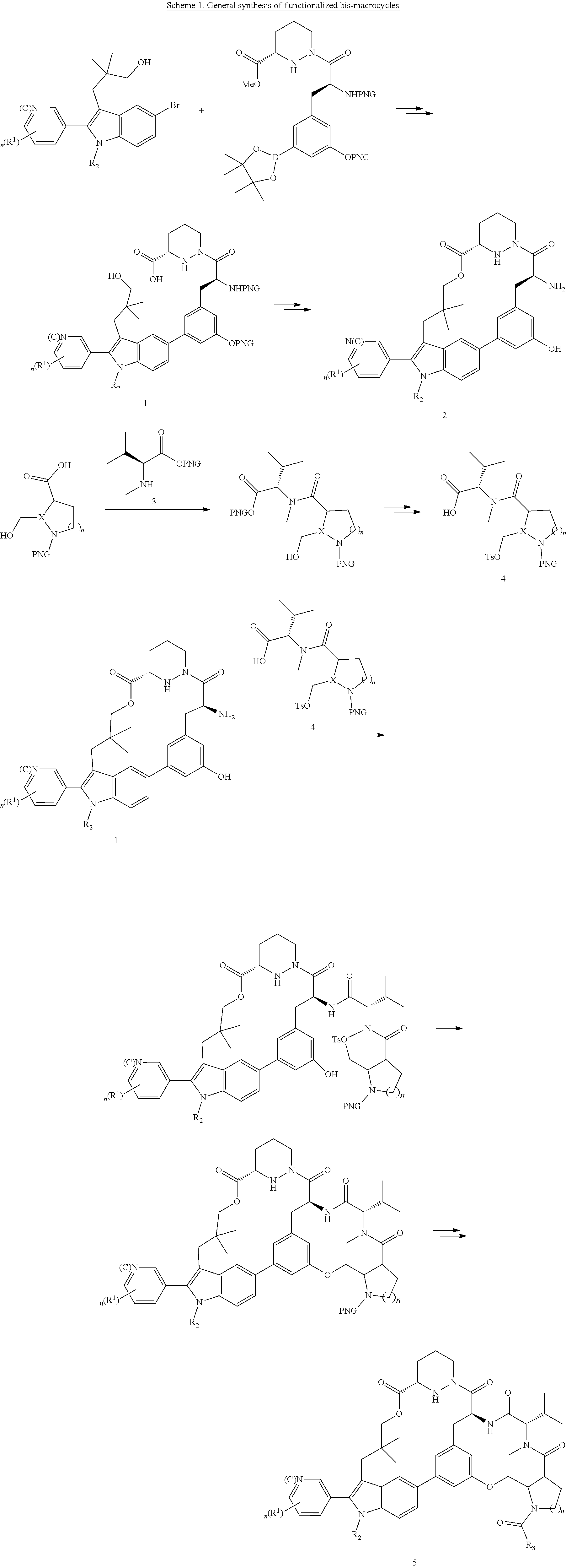

A general synthesis of functionalized bis-macrocycles is outlined in Scheme 1. An appropriately substituted biaryl intermediate (1) can be prepared in one step from an appropriately substituted 3-(5-bromo-2-iodo-1H-indol-3-yl)-2,2-dimethylpropan-1-ol intermediate and an appropriately substituted methyl piperazic ester-containing aryl boronic ester by a palladium mediated coupling followed by ester hydrolysis. Macrolactonization followed by amine and phenol deprotection can yield macrocyclic ester (2).

An appropriately substituted 2-(tosyloxymethyl)-3-(amido) cyclic amine (3) can be prepared by the coupling of an O-protected N-methyl-L-valine (4) with an appropriately substituted 2-(hydroxymethyl)-3-carboxylate cyclic amine using a peptide coupling reagent, followed by tosylation of the alcohol and carboxylic acid deprotection.

The final functionalized bis-macrocycles can then be made by the peptide coupling of macrocyclic ester (1) with intermediate (3) followed by macrocyclic ether formation in the presence of base. Deprotection and coupling of the amine with an appropriately substituted carboxylic acid (or other coupling partner) results in a bis-macrocyclic product (5).

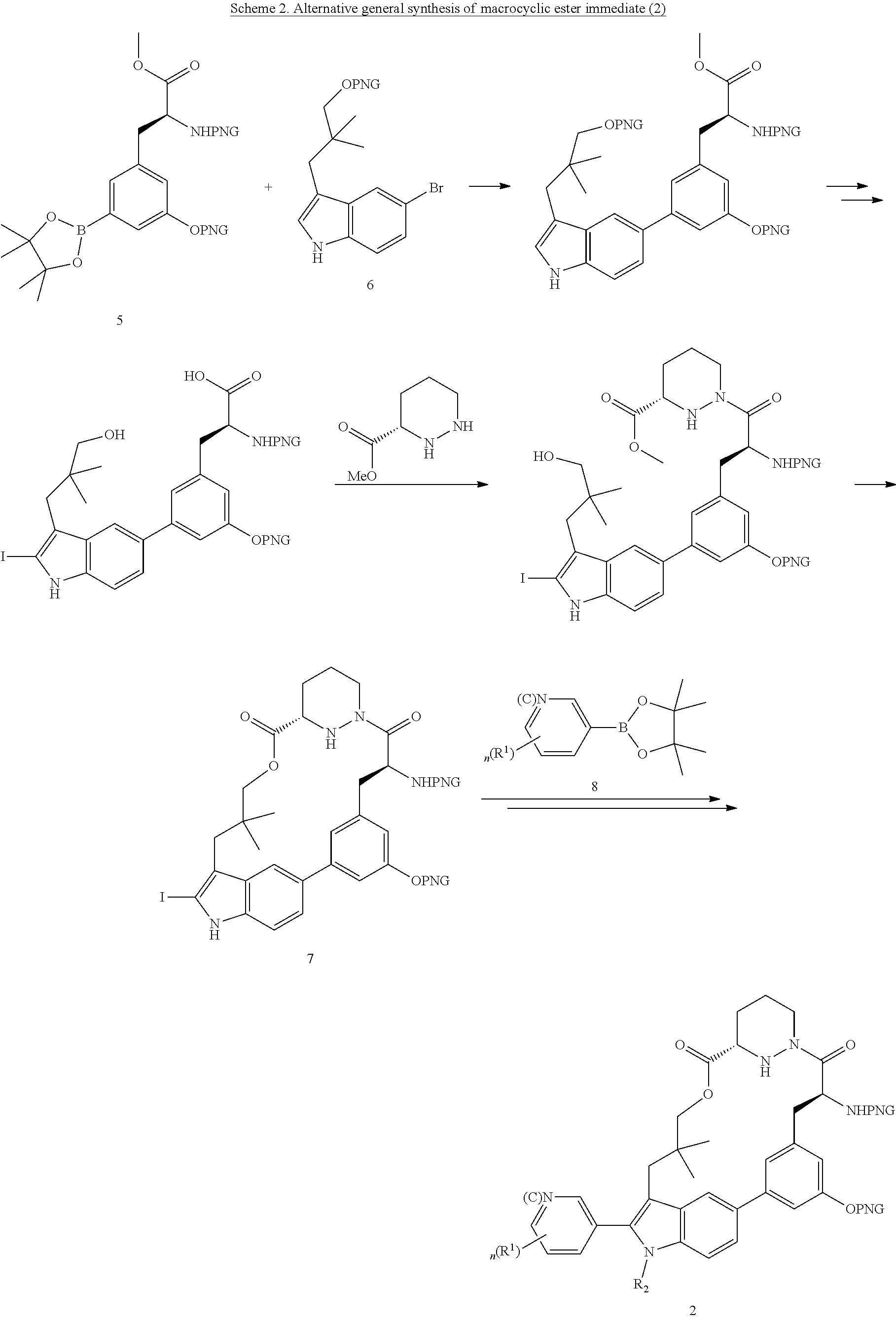

Alternatively, macrocyclic ester intermediate (2) can be prepared as described in Scheme 2. An appropriately substituted aryl boronic ester (5) and be coupled with an appropriately protected 3-(5-bromo-indol-3-yl)-2,2-dimethylpropan-1-ol (6) in the presence of a palladium catalyst. This can be followed by indole iodination, alcohol deprotection, and ester hydrolysis. Subsequent coupling with methyl (S)-piperazic ester, ester hydrolysis and macrolactonization can result in iodinated macrocyclic intermediate (7). Coupling in the presence of palladium catalyst with an appropriately substituted aryl boronic ester (8) and indole N-alkylation, followed by subsequent amine and phenol deprotection result in intermediate (2).

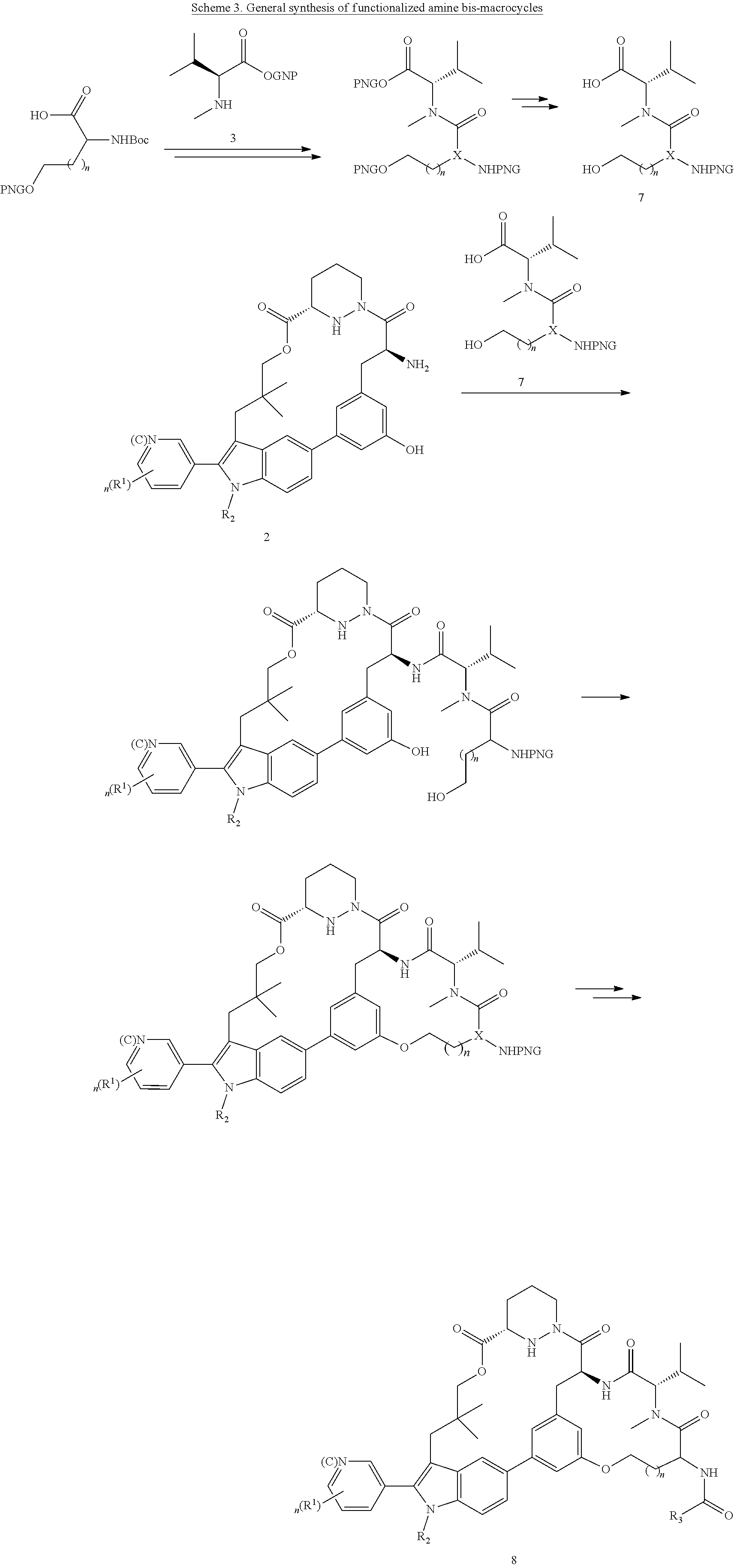

A general synthesis of functionalized bis-macrocycles is outlined in Scheme 3. An appropriately protected hydroxyalkyl amino acid can be coupled with O-protected N-methyl-L-valine (3) by a peptide coupling reagent. Subsequent alcohol and carboxylic acid deprotection can produce appropriately substituted intermediate (7).

A protected amine bis-macrocyclic intermediate can be made by peptide coupling of macrocyclic ester intermediate (2) with carboxylic acid (7) followed by bis-macrocyclic ether formation in the presence of triphenylphosphine and an azodicarboxylate. Deprotection and coupling of the amine with an appropriately substituted carboxylic acid (or other coupling partner) results in final bis-macrocyclic product (8).

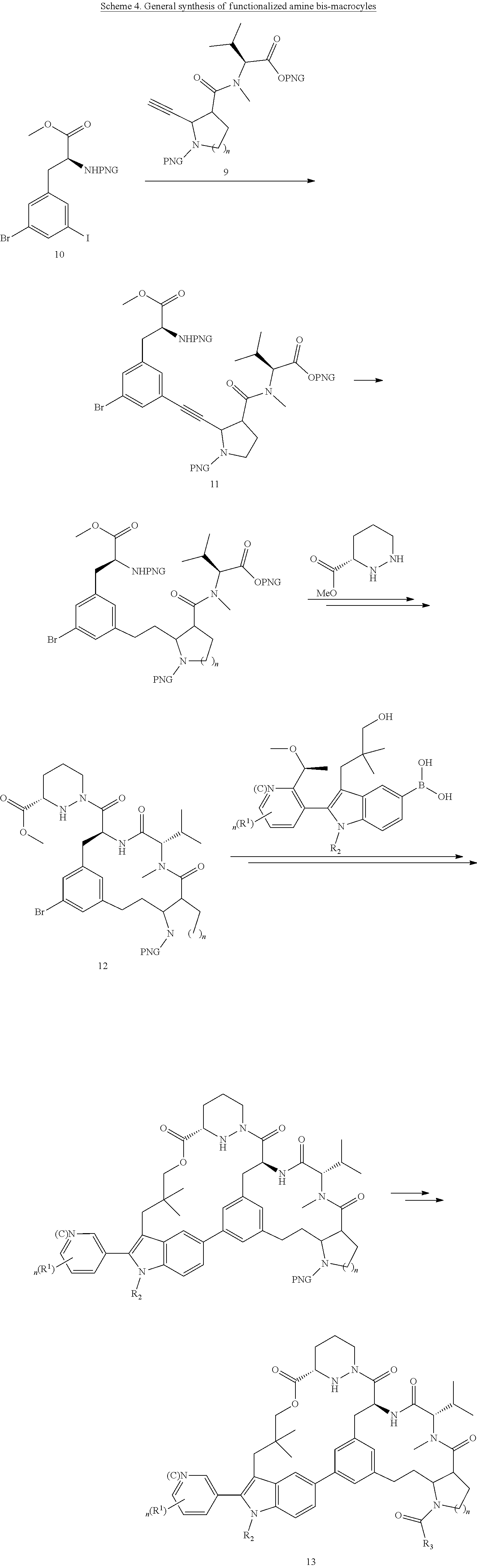

A general synthesis of functionalized bis-macrocycles is outlined in Scheme 4. An appropriately substituted terminal alkyne (9) can be coupled with an appropriately substituted iodinated bromoarene (10) in the presence of a palladium catalyst. Subsequent reduction of aryl alkyne intermediate (11) followed by amino acid N-deprotection, carboxylic acid deprotection, macrocyclization in the presence of a peptide coupling reagent, ester hydrolysis, and peptide coupling with methyl (S)-piperazic ester can yield macrocyclic intermediate (12). Functionalized bis-macrocycle (13) can then be obtained through a palladium mediated coupling with an appropriately substituted 3-(5-boronate-indol-3-yl)-2,2-dimethylpropan-1-ol, macrolactonization, amine deprotection, and coupling of the amine with an appropriately substituted carboxylic acid (or other coupling partner).

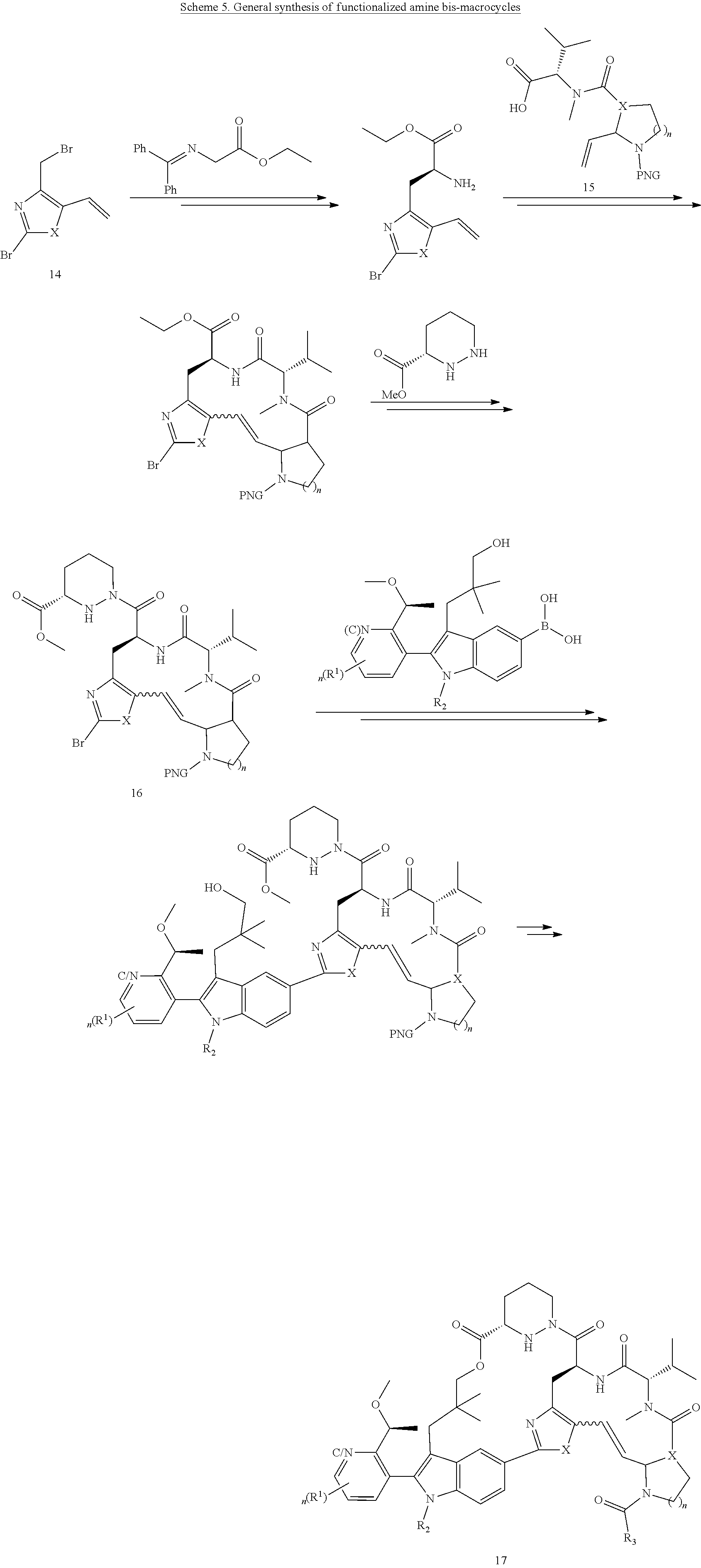

A general synthesis of functionalized bis-macrocycles is outlined in Scheme 5. An appropriately substituted 2-bromo-4-bromomethyl-5-ethenyl 5-membered heteroarene (14) can react with ethyl 2-((diphenylethylene)amino) acetate in the presence of base and a chiral auxiliary. Subsequent amide coupling with an appropriately substituted 2-(ethenyl)-3-(amido) cyclic amine (15) followed by an olefin metathesis reaction, ester hydrolysis, and an amide coupling reaction with methyl (S)-piperazic ester can yield macrocycle (16).

Functionalized amine bis-macrocycle (17) can then be obtained through a palladium mediated coupling with an appropriately substituted 3-(5-boronate-indol-3-yl)-2,2-dimethylpropan-1-ol, methyl ester hydrolysis, macrolactonization, amine deprotection, and coupling of the amine with an appropriately substituted carboxylic acid (or other coupling partner).

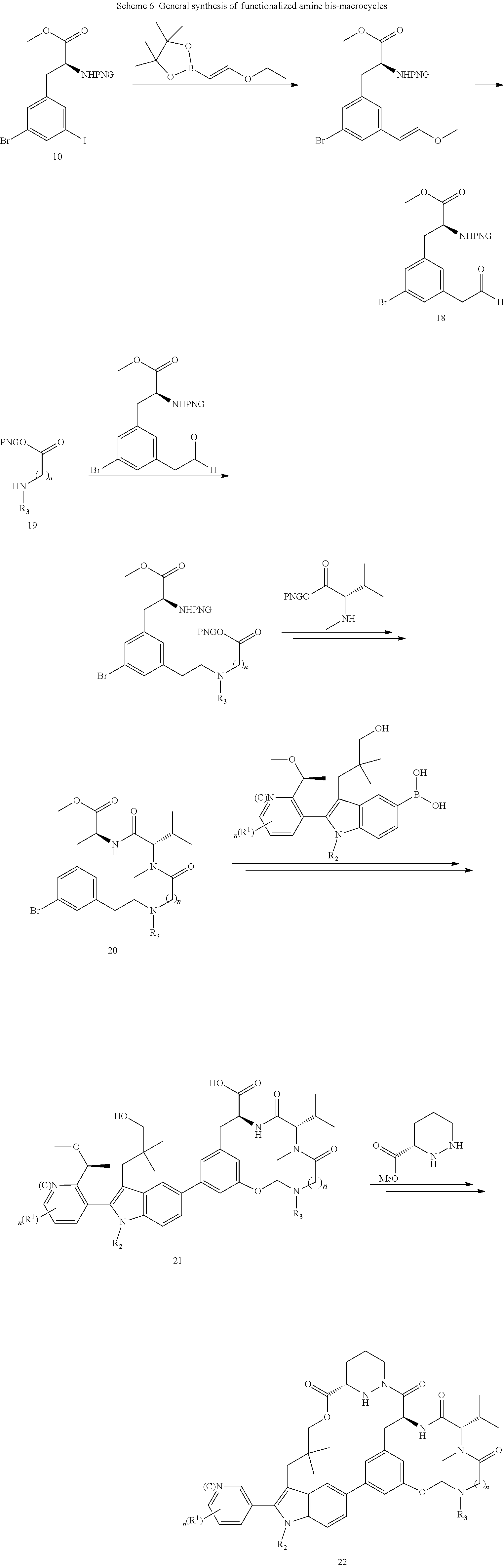

A general synthesis of functionalized bis-macrocycles is outlined in Scheme 6. An appropriately substituted iodinated bromoarene (10) can be coupled with a vinyl boronate ester in the presence of a palladium catalyst. Hydrolysis of the vinyl ether in the presence of acid can yield aldehyde (18).

An appropriately N-functionalized O-protected amino acid (19) can be coupled with an aldehyde (18) in the presence of acid and a reducing agent. This can be followed by carboxylic acid deprotection and coupling of an O-protected N-methyl-L-valine (3) in the presence of an amide coupling reagent. Subsequent carboxylate and amine deprotections followed by cyclization in the presence of a peptide coupling reagent hydrolysis can yield macrocyclic intermediate (20).

An appropriately substituted biaryl intermediate (21) can then be prepared in two steps through coupling of intermediate (20) with an appropriately substituted 3-(5-boronate-indol-3-yl)-2,2-dimethylpropan-1-ol by a palladium mediated coupling followed by ester hydrolysis. Subsequent coupling with methyl (S)-piperazic ester by a peptide coupling reagent, ester hydrolysis, and macrolactonization can yield functionalized amine bis-macrocycle (22).

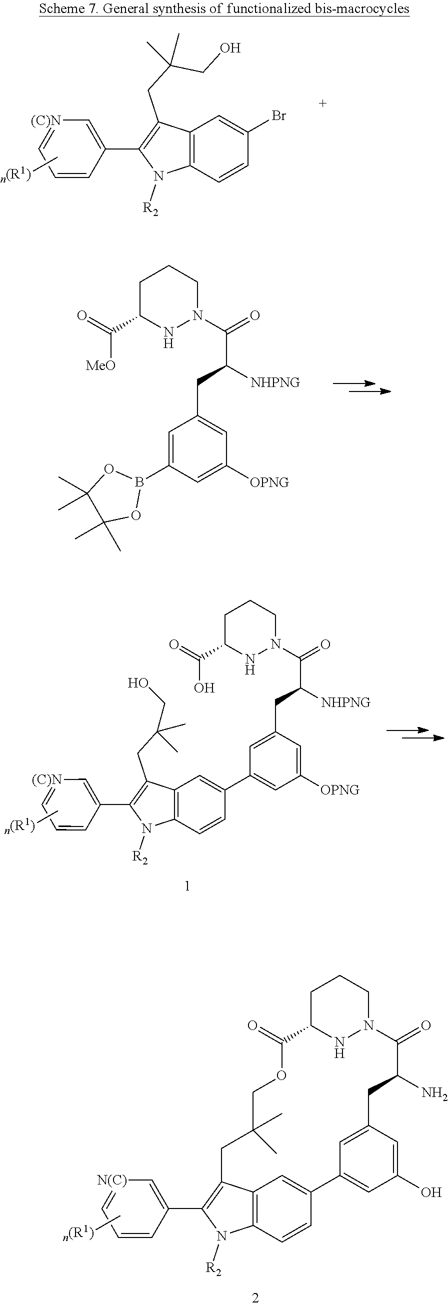

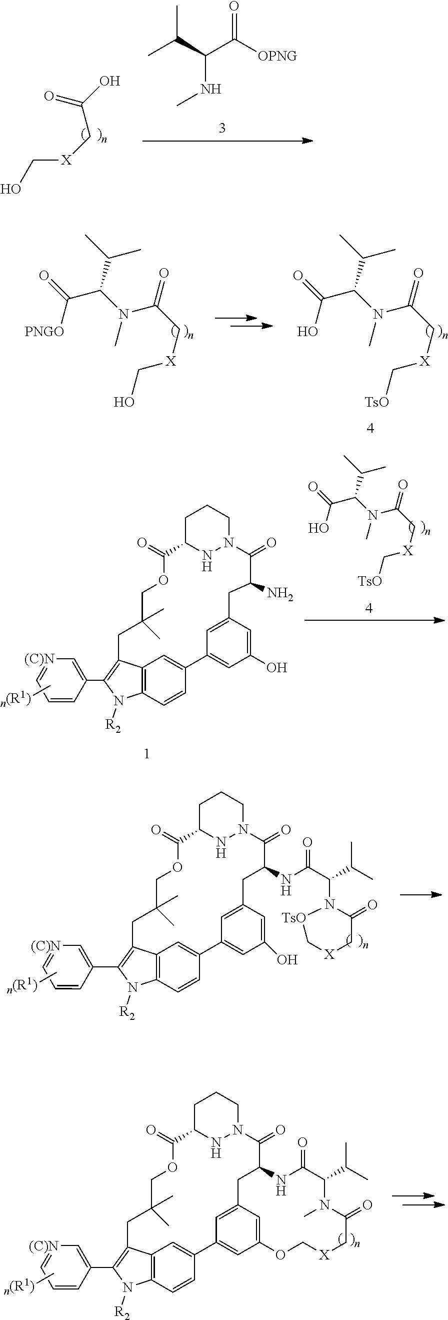

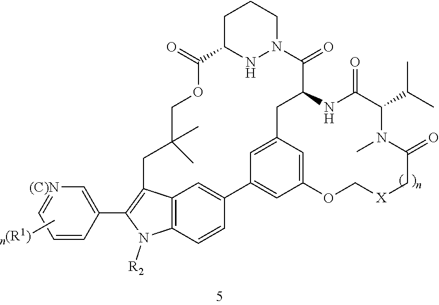

A general synthesis of functionalized bis-macrocycles is outlined in Scheme 7. An appropriately substituted biaryl intermediate (1) can be prepared in one step from an appropriately substituted 3-(5-bromo-2-iodo-1H-indol-3-yl)-2,2-dimethylpropan-1-ol intermediate and an appropriately substituted methyl piperazic ester-containing aryl boronic ester by a palladium mediated coupling followed by ester hydrolysis. Macrolactonization followed by amine and phenol deprotection can yield macrocyclic ester (2).

An appropriately substituted tosylated alcohol (4) can be prepared by the coupling of an 0-protected N-methyl-L-valine (3) with an appropriately substituted hydroxyl carboxylate using a peptide coupling reagent, followed by tosylation of the alcohol and carboxylic acid deprotection.

The final functionalized bis-macrocycles can then be made by the peptide coupling of macrocyclic ester (1) with intermediate (3) followed by macrocyclic ether formation in the presence of base. Deprotection and coupling of the amine with an appropriately substituted carboxylic acid (or other coupling partner) results in a bis-macrocyclic product (5).

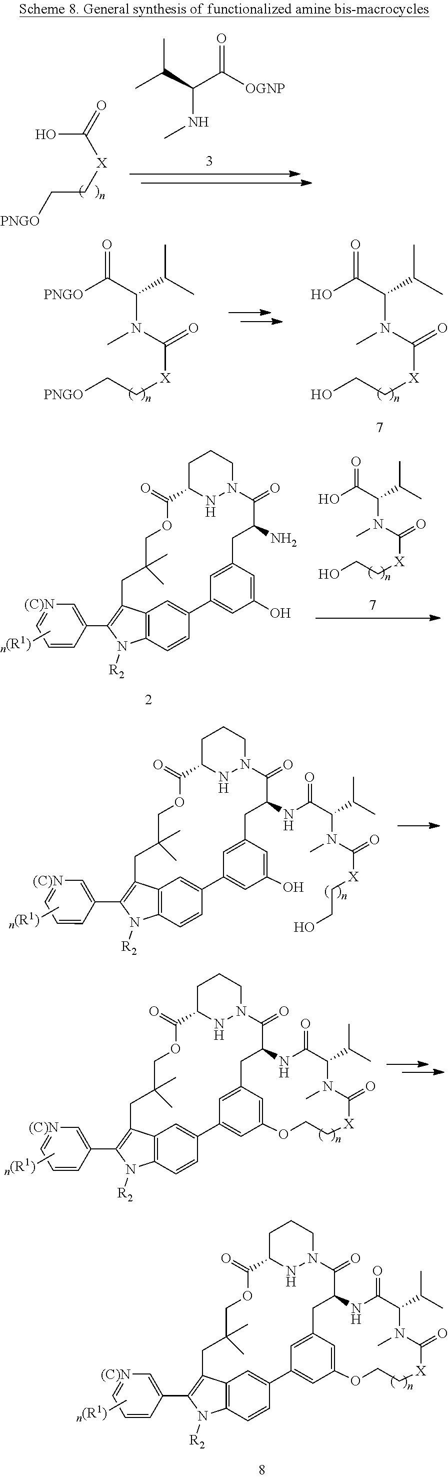

A general synthesis of functionalized bis-macrocycles is outlined in Scheme 8. An appropriately protected hydroxyalkyl carboxylic acid can be coupled with O-protected N-methyl-L-valine (3) by a peptide coupling reagent. Subsequent alcohol and carboxylic acid deprotection can produce appropriately substituted intermediate (7).

A protected amine bis-macrocyclic intermediate can be made by peptide coupling of macrocyclic ester intermediate (2) with carboxylic acid (7) followed by bis-macrocyclic ether formation in the presence of triphenylphosphine and an azodicarboxylate. Deprotection and coupling of the amine with an appropriately substituted carboxylic acid (or other coupling partner) results in final bis-macrocyclic product (8).

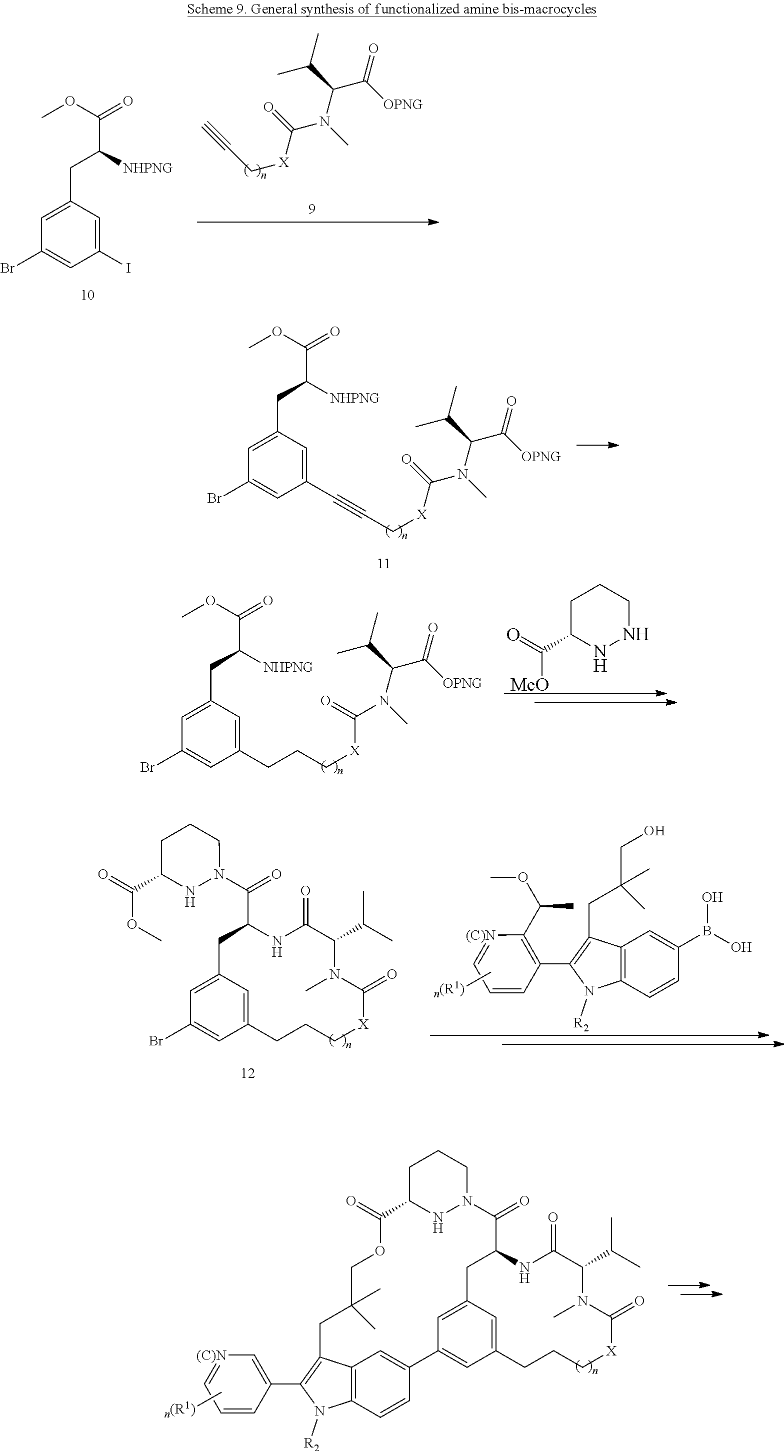

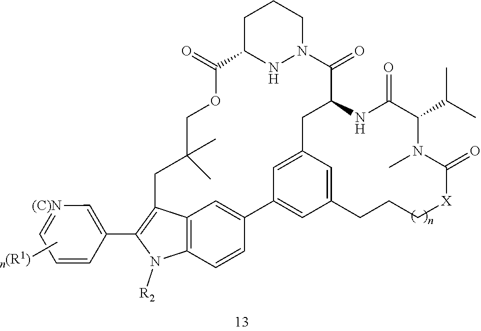

A general synthesis of functionalized bis-macrocycles is outlined in Scheme 9. An appropriately substituted terminal alkyne (9) can be coupled with an appropriately substituted iodinated bromoarene (10) in the presence of a palladium catalyst. Subsequent reduction of aryl alkyne intermediate (11) followed by amino acid N-deprotection, carboxylic acid deprotection, macrocyclization in the presence of a peptide coupling reagent, ester hydrolysis, and peptide coupling with methyl (S)-piperazic ester can yield macrocyclic intermediate (12). Functionalized bis-macrocycle (13) can then be obtained through a palladium mediated coupling with an appropriately substituted 3-(5-boronate-indol-3-yl)-2,2-dimethylpropan-1-ol, macrolactonization, amine deprotection, and coupling of the amine with an appropriately substituted carboxylic acid (or other coupling partner).

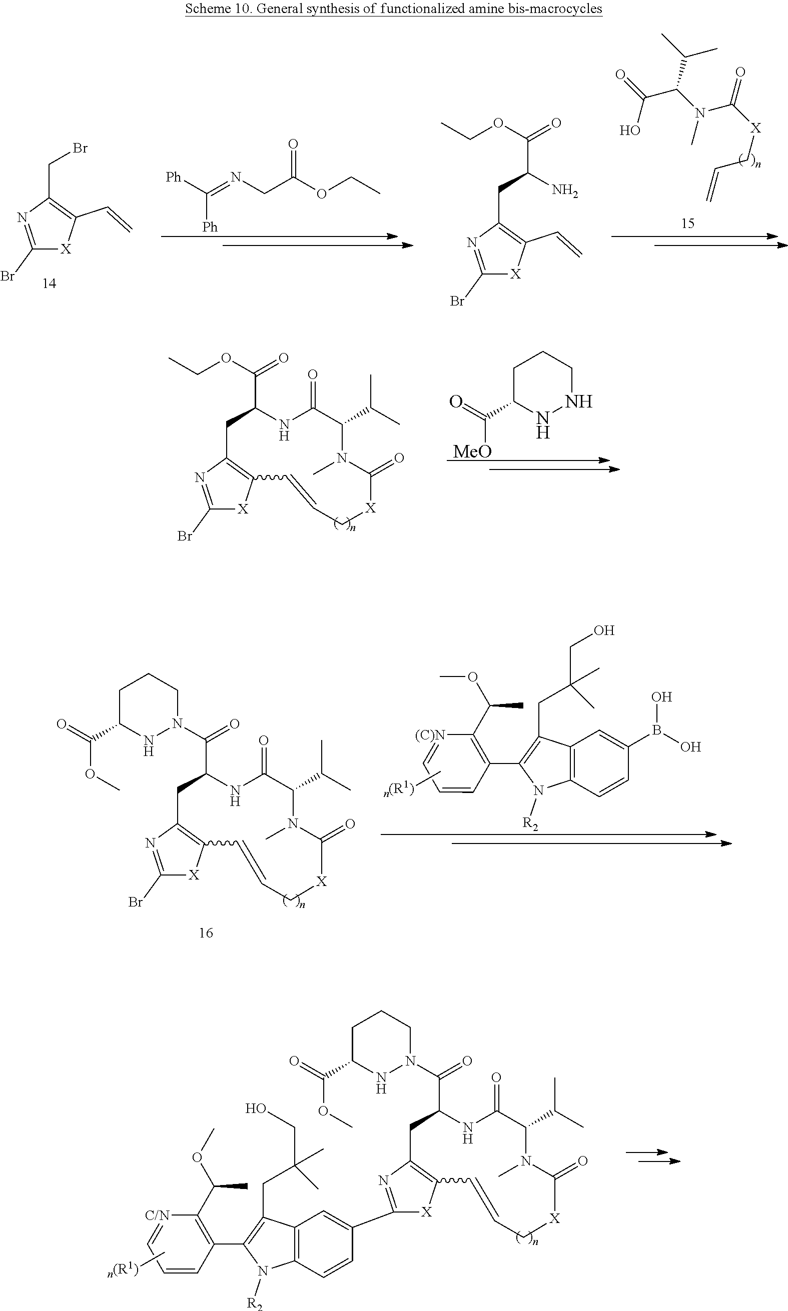

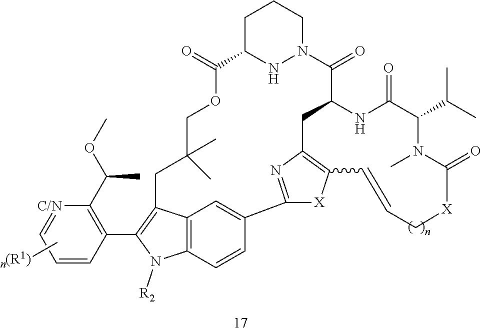

A general synthesis of functionalized bis-macrocycles is outlined in Scheme 10. An appropriately substituted 2-bromo-4-bromomethyl-5-ethenyl 5-membered heteroarene (14) can react with ethyl 2-((diphenylethylene)amino) acetate in the presence of base and a chiral auxiliary. Subsequent amide coupling with an appropriately substituted alkenyl valine (15) followed by an olefin metathesis reaction, ester hydrolysis, and an amide coupling reaction with methyl (S)-piperazic ester can yield macrocycle (16).

Functionalized amine bis-macrocycle (17) can then be obtained through a palladium mediated coupling with an appropriately substituted 3-(5-boronate-indol-3-yl)-2,2-dimethylpropan-1-ol, methyl ester hydrolysis, macrolactonization, amine deprotection, and coupling of the amine with an appropriately substituted carboxylic acid (or other coupling partner).

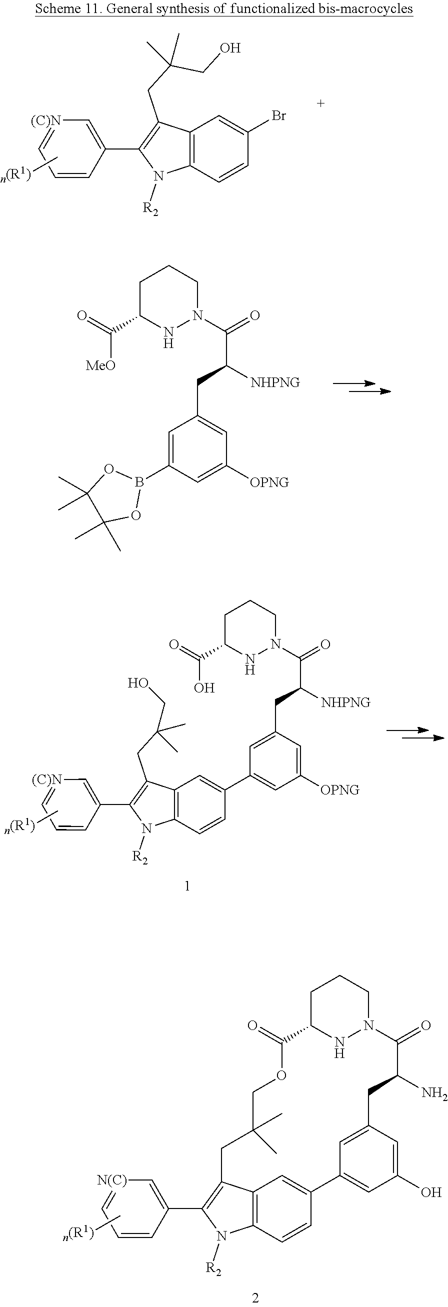

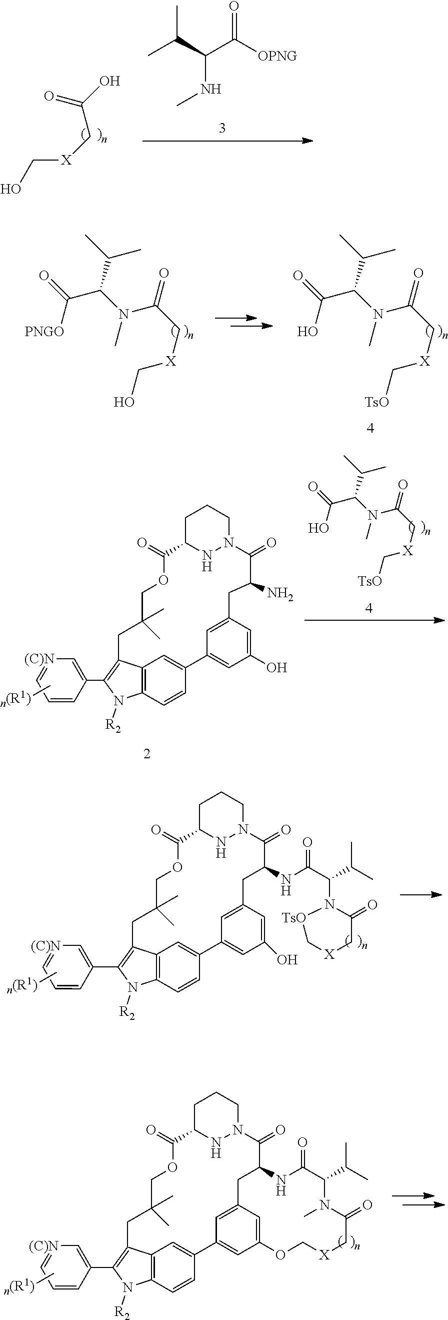

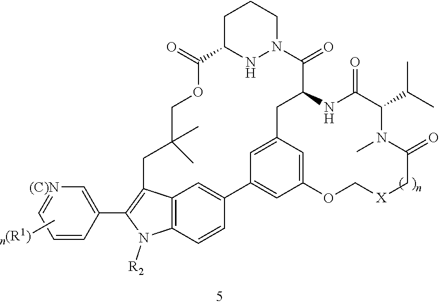

A general synthesis of functionalized bis-macrocycles is outlined in Scheme 1. An appropriately substituted biaryl intermediate (1) can be prepared in one step from an appropriately substituted 3-(5-bromo-2-iodo-1H-indol-3-yl)-2,2-dimethylpropan-1-ol intermediate and an appropriately substituted methyl piperazic ester-containing aryl boronic ester by a palladium mediated coupling followed by ester hydrolysis. Macrolactonization followed by amine and phenol deprotection can yield macrocyclic ester (2).

An appropriately substituted tosylated alcohol (4) can be prepared by the coupling of an 0-protected N-methyl-L-valine (3) with an appropriately substituted hydroxyl carboxylate using a peptide coupling reagent, followed by tosylation of the alcohol and carboxylic acid deprotection.

The final functionalized bis-macrocycles can then be made by the peptide coupling of macrocyclic ester (2) with intermediate (4) followed by macrocyclic ether formation in the presence of base. Deprotection and coupling of the amine with an appropriately substituted carboxylic acid (or other coupling partner) results in a bis-macrocyclic product (5).

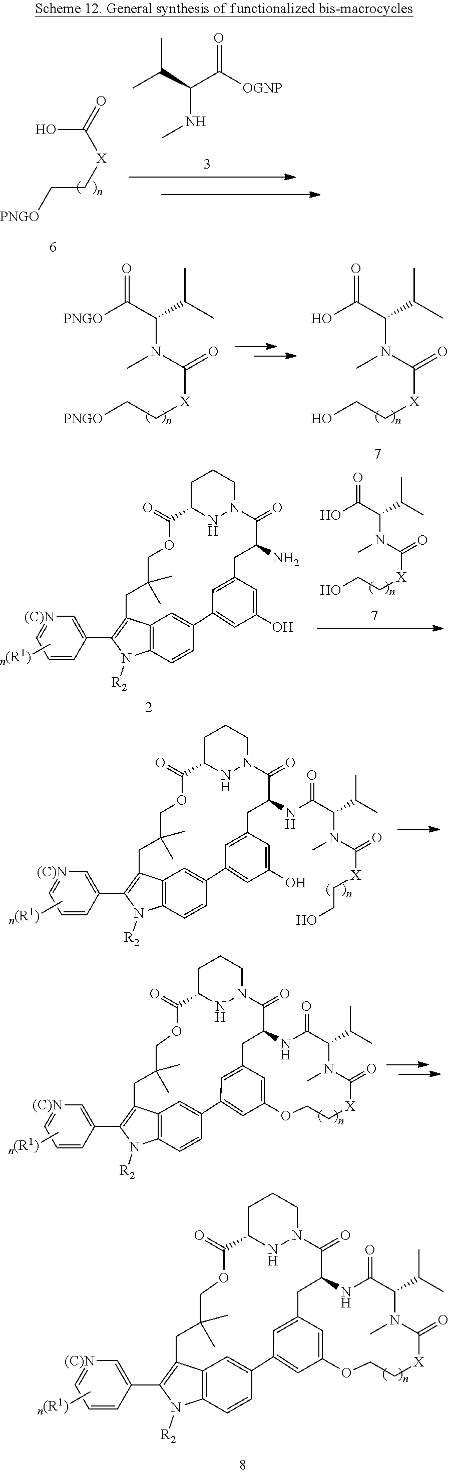

A general synthesis of functionalized bis-macrocycles is outlined in Scheme 2. An appropriately protected hydroxyalkyl carboxylic acid (6) can be coupled with O-protected N-methyl-L-valine (3) by a peptide coupling reagent. Subsequent alcohol and carboxylic acid deprotection can produce appropriately substituted intermediate (7).

A bis-macrocyclic intermediate can be made by peptide coupling of macrocyclic ester intermediate (2) with carboxylic acid intermediate (7) followed by bis-macrocyclic ether formation in the presence of triphenylphosphine and an azodicarboxylate. Deprotection and coupling of the amine with an appropriately substituted carboxylic acid (or other coupling partner) results in final bis-macrocyclic product (8).

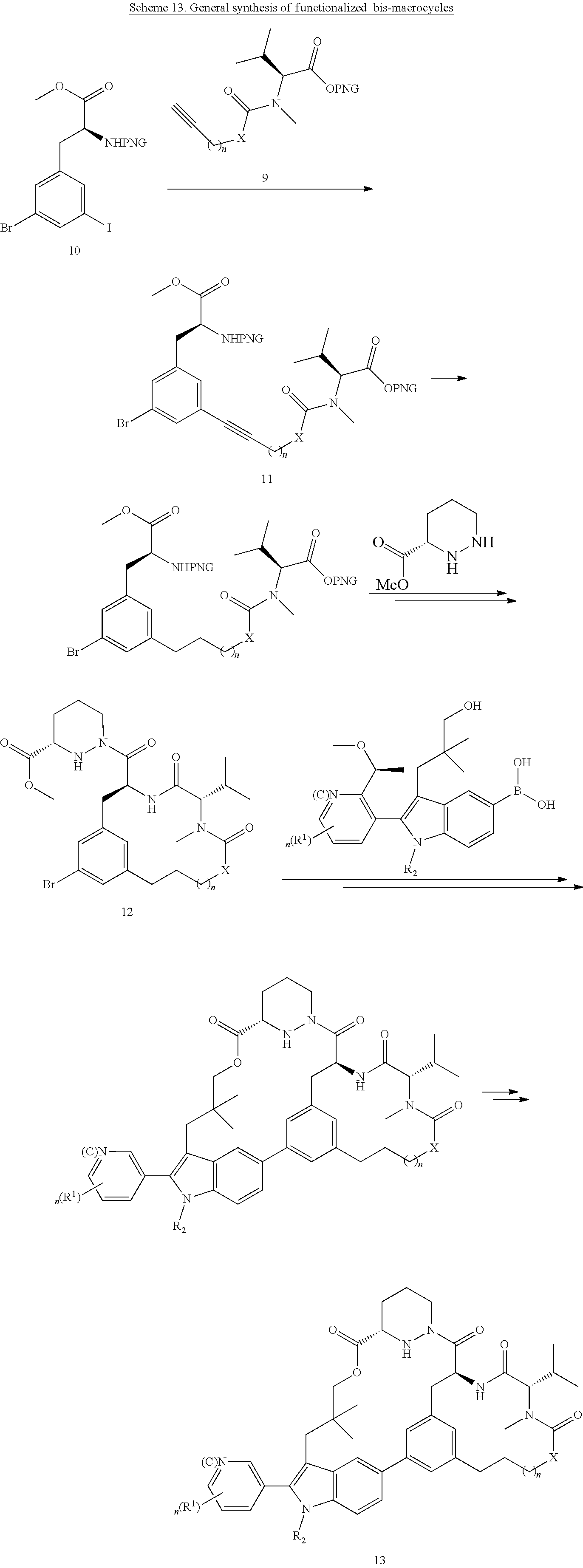

A general synthesis of functionalized bis-macrocycles is outlined in Scheme 3. An appropriately substituted terminal alkyne (9) can be coupled with an appropriately substituted iodinated bromoarene (10) in the presence of a palladium catalyst. Subsequent reduction of aryl alkyne intermediate (11) followed by amino acid N-deprotection, carboxylic acid deprotection, macrocyclization in the presence of a peptide coupling reagent, ester hydrolysis, and peptide coupling with methyl (S)-piperazic ester can yield macrocyclic intermediate (12). Functionalized bis-macrocycle (13) can then be obtained through a palladium mediated coupling with an appropriately substituted 3-(5-boronate-indol-3-yl)-2,2-dimethylpropan-1-ol, macrolactonization, amine deprotection, and coupling of the amine with an appropriately substituted carboxylic acid (or other coupling partner).

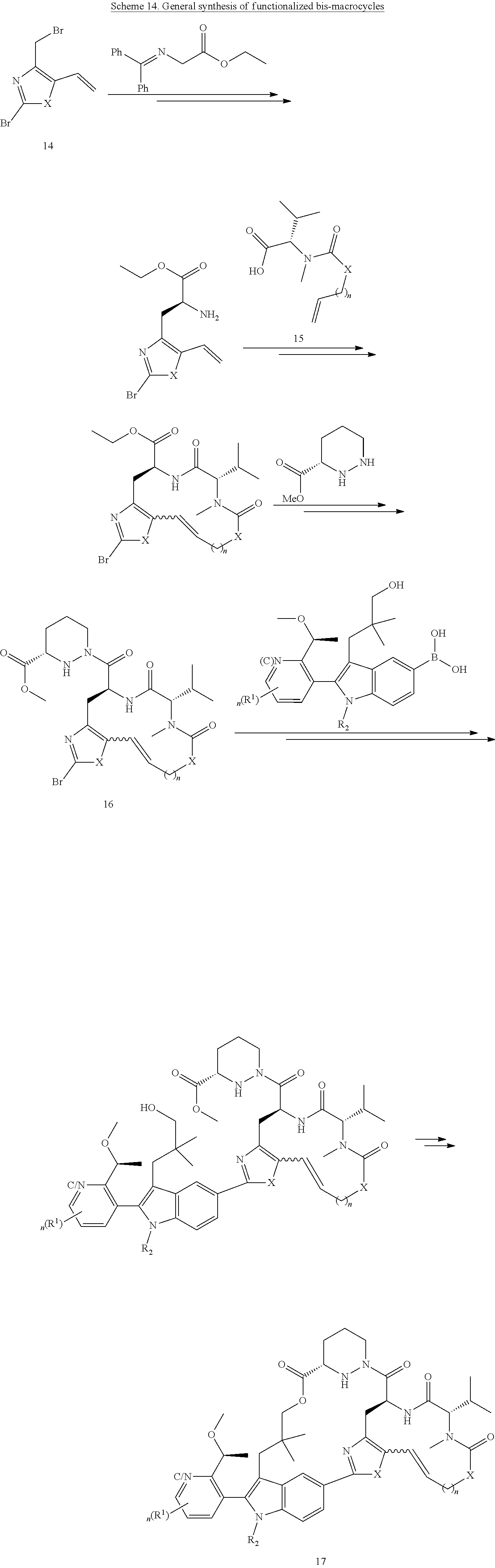

A general synthesis of functionalized bis-macrocycles is outlined in Scheme 4. An appropriately substituted 2-bromo-4-bromomethyl-5-ethenyl 5-membered heteroarene (14) can react with ethyl 2-((diphenylethylene)amino) acetate in the presence of base and a chiral auxiliary. Subsequent amide coupling with an appropriately substituted alkenyl valine (15) followed by an olefin metathesis reaction, ester hydrolysis, and an amide coupling reaction with methyl (S)-piperazic ester can yield macrocycle (16).

Functionalized bis-macrocycle (17) can then be obtained through a palladium mediated coupling with an appropriately substituted 3-(5-boronate-indol-3-yl)-2,2-dimethylpropan-1-ol, methyl ester hydrolysis, macrolactonization, amine deprotection, and coupling of the amine with an appropriately substituted carboxylic acid (or other coupling partner).

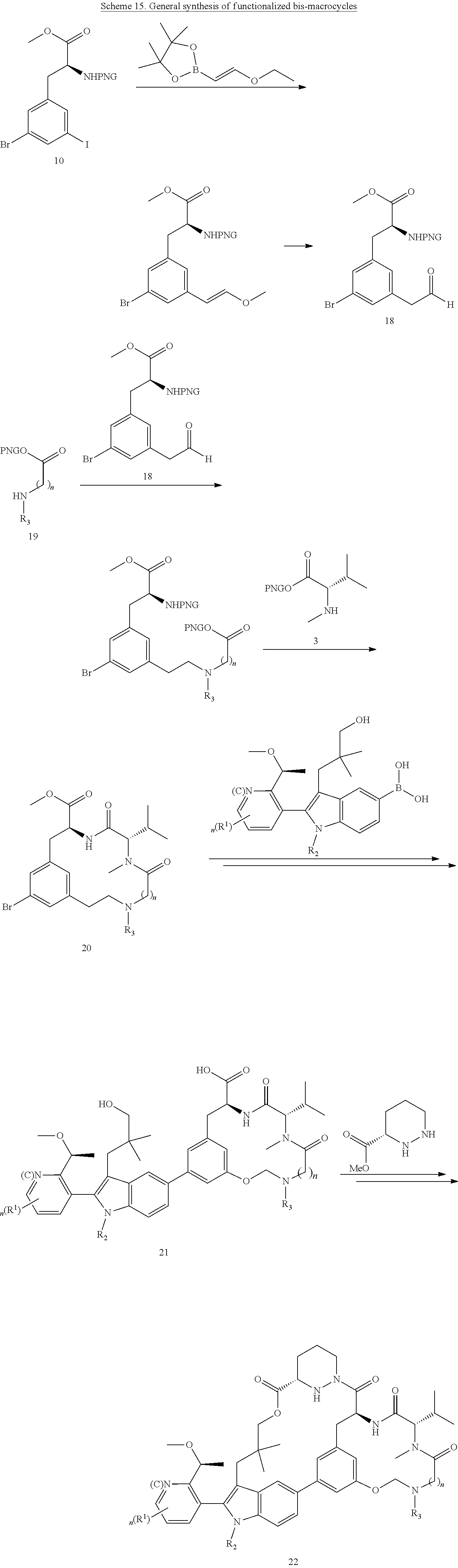

A general synthesis of functionalized bis-macrocycles is outlined in Scheme 5. An appropriately substituted iodinated bromoarene (10) can be coupled with a vinyl boronate ester in the presence of a palladium catalyst. Hydrolysis of the vinyl ether in the presence of acid can yield aldehyde (18).

An appropriately N-functionalized O-protected amino acid (19) can be coupled with aldehyde (18) in the presence of acid and a reducing agent. This can be followed by carboxylic acid deprotection and coupling of an O-protected N-methyl-L-valine (3) in the presence of an amide coupling reagent. Subsequent carboxylate and amine deprotections followed by cyclization in the presence of a peptide coupling reagent can yield macrocyclic intermediate (20).

An appropriately substituted biaryl intermediate (21) can then be prepared in two steps through coupling of intermediate (20) with an appropriately substituted 3-(5-boronate-indol-3-yl)-2,2-dimethylpropan-1-ol by a palladium mediated coupling followed by ester hydrolysis. Subsequent coupling with methyl (S)-piperazic ester by a peptide coupling reagent, ester hydrolysis, and macrolactonization can yield functionalized amine bis-macrocycle (22).

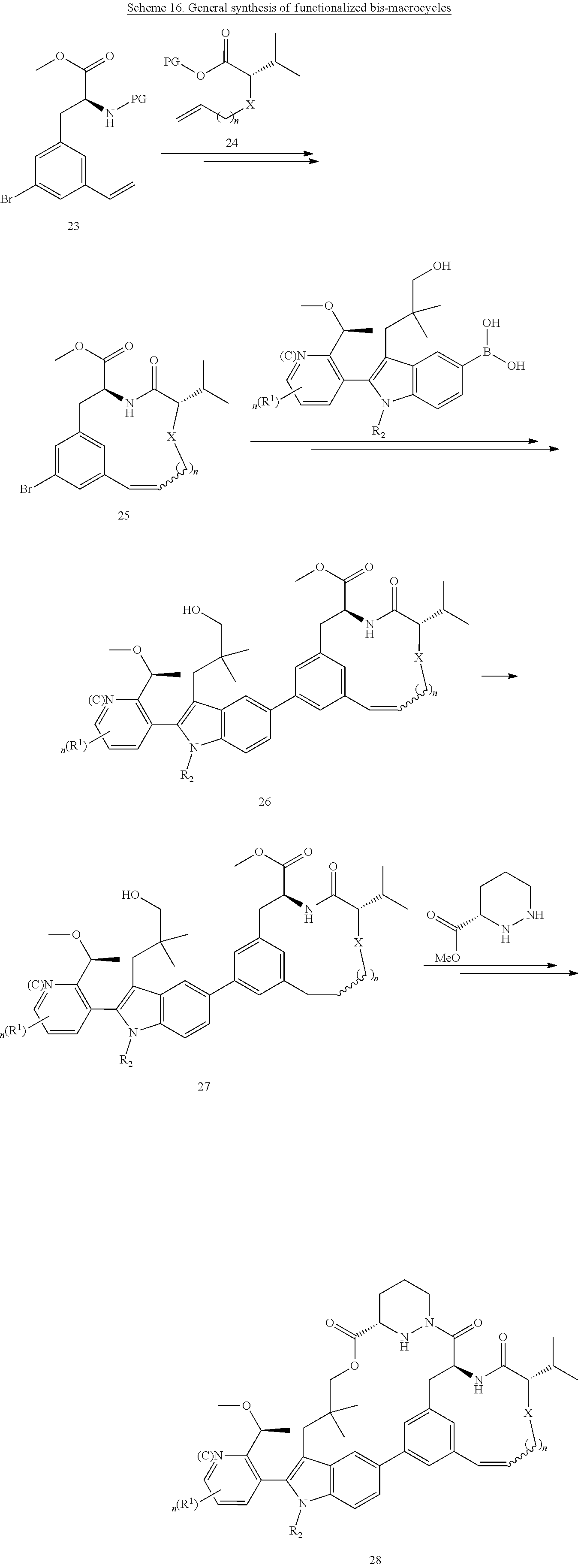

A general synthesis of functionalized bis-macrocycles is outlined in Scheme 6. An appropriately substituted vinyl bromoarene (23) can be coupled with an appropriately functionalized O-protected carboxylic acid containing a terminal alkene (24) in the presence of an olefin metathesis catalyst. Subsequent carboxylic acid deprotection and amine deprotection followed by intramolecular coupling in the presence of a peptide coupling reagent can yield mono-macrocyclic intermediate (25).

An appropriately substituted biaryl intermediate (26) can then be prepared in two steps through coupling of intermediate (25) with an appropriately substituted 3-(5-boronate-indol-3-yl)-2,2-dimethylpropan-1-ol by a palladium mediated coupling followed by ester hydrolysis. Alkene reduction in the presence of H₂ and an appropriate transition metal hydrogenation catalyst followed by ester hydrolysis can yield intermediate (27). Subsequent coupling with methyl (S)-piperazic ester by a peptide coupling reagent, ester hydrolysis, and macrolactonization can yield functionalized bis-macrocycle (28).

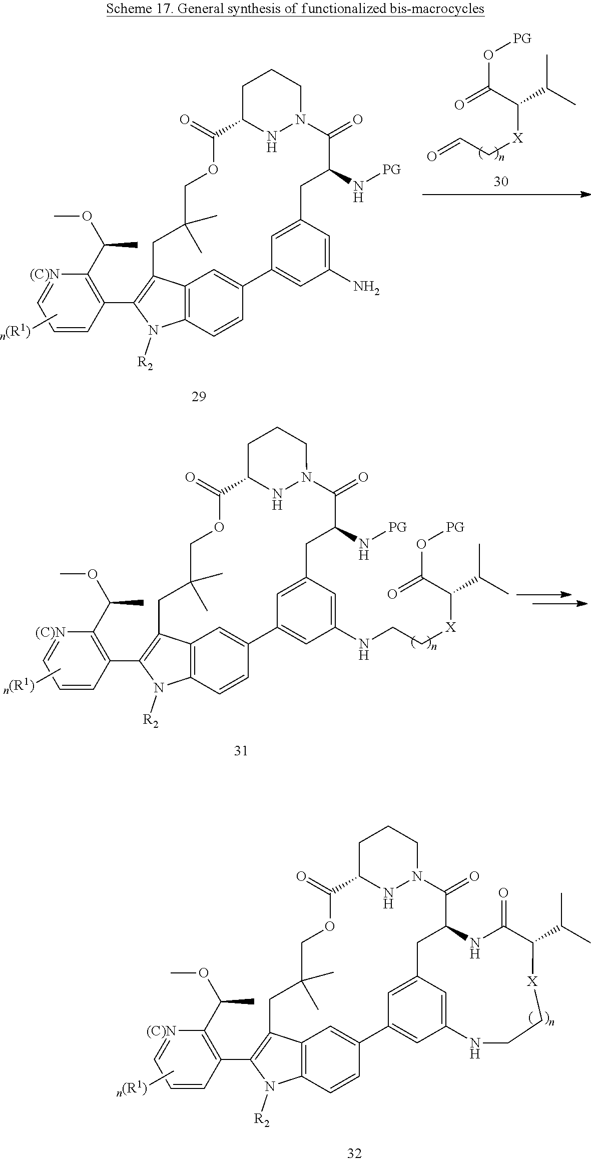

A general synthesis of functionalized bis-macrocycles is outlined in Scheme 7. An appropriately substituted aniline macrocycle (29) can be coupled with an appropriately functionalized O-protected carboxylic acid containing an aldehyde (30) by a reductive amination in the presence of acid and a reducing agent to form intermediate (31). Subsequent carboxylate and amine deprotections followed by macrocyclization in the presence of a peptide coupling reagent can yield functionalized aniline bis-macrocycle (32).

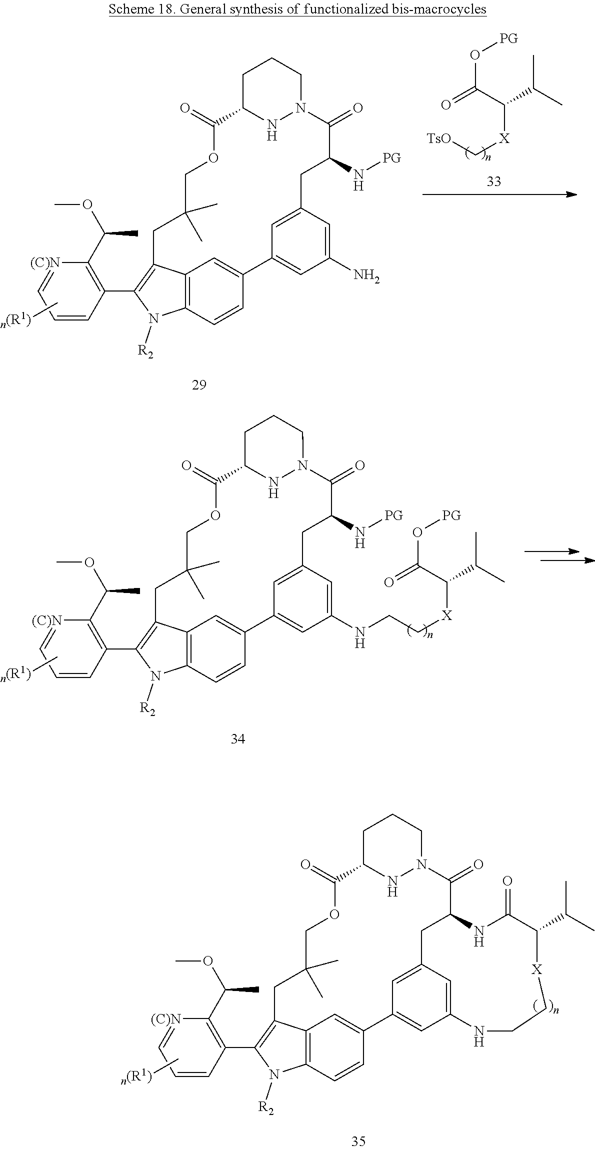

A general synthesis of functionalized bis-macrocycles is outlined in Scheme 8. An appropriately substituted aniline macrocycle (29) can be coupled with an appropriately functionalized O-protected carboxylic acid containing a tosylate (33) in the presence of base to form intermediate (34). Subsequent carboxylate and amine deprotections followed by macrocyclization in the presence of a peptide coupling reagent can yield aniline bis-macrocycle (35).

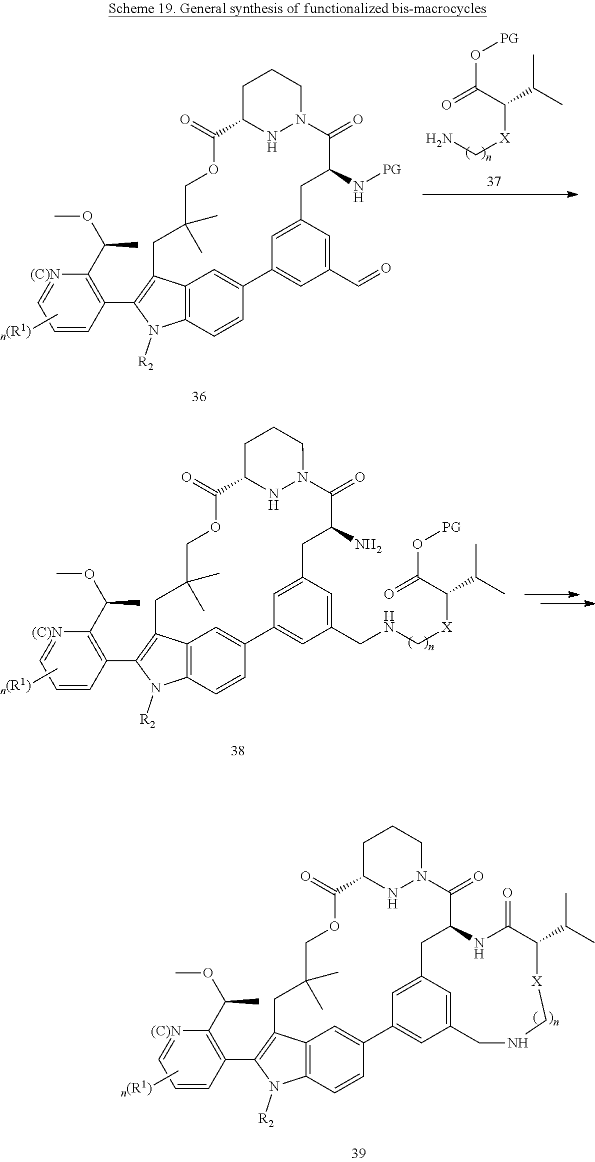

A general synthesis of functionalized bis-macrocycles is outlined in Scheme 9. An appropriately substituted benzaldehyde macrocycle (36) can be coupled with an appropriately functionalized 0-protected carboxylic acid containing an amine (37) through a reductive amination in the presence of acid and a reducing agent to form intermediate (38). Subsequent carboxylate and amine deprotections followed by macrocyclization in the presence of a peptide coupling reagent can yield functionalized benzylic amine bis-macrocycle (39).

In any embodiment herein, a compound of the present invention may be modified with a substituent as found in any one or more of the following applications using methodologies described in these applications in combination with methods provided herein and know to those of skill in the art: WO 2024/060966, WO 2024/017859, WO 2024/008834, WO 2024/008610, WO 2023/232776, WO 2023/208005, WO 2023/086341, WO 2023/025832, WO 2023/015559, CN 117720556, CN 117720555, CN 117720554, CN 117534687, CN 117534685 and CN 117534684, each incorporated herein by reference in their entireties.

### Pharmaceutical Compositions and Methods of Use

The compounds with which the invention is concerned are Ras inhibitors and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-V C H, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder, or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, di-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

In some embodiments, the invention discloses a method of treating a disease or disorder that is characterized by aberrant Ras activity due to a Ras mutant. In some embodiments, the disease or disorder is a cancer.

Accordingly, also provided is a method of treating cancer in a subject in need thereof, the method comprising administering to the subject a therapeutically effective amount of a compound of the present invention, or a pharmaceutically acceptable salt thereof, or a pharmaceutical composition comprising such a compound or salt. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a Ras protein-related disorder in a subject in need thereof, the method comprising administering to the subject a therapeutically effective amount of a compound of the present invention, or a pharmaceutically acceptable salt thereof, or a pharmaceutical composition comprising such a compound or salt.

In some embodiments, the compounds of the present invention or pharmaceutically acceptable salts thereof, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate, and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma, and teratoma; Lung, for example: bronchogenic carcinoma (squamous cell, undifferentiated small cell,
undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the Ras protein is wild type (Ras^{WT}). Accordingly, in some embodiments, a compound of the present invention is employed in a method of treating a patient having a cancer comprising a Ras^{WT} (e.g., K-Ras^{WT}, H-Ras^{WT} or N-Ras^{WT}). In some embodiments, the Ras protein is Ras amplification (e.g., K-Ras^{amp}). Accordingly, in some embodiments, a compound of the present invention is employed in a method of treating a patient having a cancer comprising a Ras^{amp} (K-Ras^{amp}, H-Ras^{amp} or N-Ras^{amp}). In some embodiments, the cancer comprises a Ras mutation, such as a Ras mutation described herein. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof;
   or a combination of any of the foregoing. In some embodiments, the cancer comprises a K-Ras mutation selected from the group consisting of G12C, G12D, G13C, G12V, G13D, G12R, G12S, Q61H, Q61K, Q61R and Q61 L. In some embodiments, the cancer comprises a K-ras mutation that is Q61H. In some embodiments, the cancer comprises an N-Ras mutation selected from the group consisting of G12C, Q61H, Q61K, Q61L, Q61P and Q61R. In some embodiments, the cancer comprises an H-Ras mutation selected from the group consisting of Q61H and Q61L. In some embodiments, the cancer comprises a K-ras mutation that is Q61H. In some embodiments, a compound of the present invention inhibits more than one Ras mutant. In some embodiments, a compound of the present invention inhibits Ras^{WT} in addition to one or more additional Ras mutations (e.g., K-, H- or N-Ras^{WT} and K-Ras G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or N-Ras^{WT} and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{WT} and N-Ras Q61R, Q61K, G12D, Q61 L, Q61H, G13R, G13D, G12S, G12C, G12V, G12A, G13V, G12R, P185S, G13C, A146T, G60E, Q61P, A59D, E132K, E49K, T50I, A146V, or A59T). In some embodiments, a compound of the present invention inhibits Ras^{amp} in addition to one or more additional Ras mutations (e.g., K-, H- or N-Ras^{amp} and K-Ras G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or N-Ras^{amp} and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{amp} and N-Ras Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T).

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

In some embodiments, a cancer comprises a Ras Q61H mutation and a TP53, an STK11^{LOF}, a CDKN2A, a KEAP1, a CDKN2B, an MTAP, an RBM10, a SMARCA4, an ATM, a MYC, an APC, a SMAD4, a PIK3CA, an SOX9, an FBXW7, a PTEN, a FLT3, an AMER1, a CDK8, a AKT2, an RNF43, a GATA6, an SF381, an IGH, a CDKN2C, a DNMT3A, an RB1, a TRAF3, an N-Ras, a TET2, an FAF1, a BRAF, a KMT2A, an RUNX1, a PTPN11, a ETV6, an NPM1 or an MYH11 mutation. In some embodiments, the cancer is non-small cell lung cancer and comprises a K-Ras Q61H mutation and a TP53, an STK11^{LOF}, a CDKN2A, a KEAP1, a CDKN2B, an MTAP, an RBM10, a SMARCA4, an ATM, or a MYC mutation. In some embodiments, the cancer is colorectal cancer and comprises a K-Ras G61H mutation and an APC, a TP53, an SMAD4, a PIK3CA, an SOX9, an FBXW7, a PTEN, a FLT3, an AMER1 or a CDK8 mutation. In some embodiments, the cancer is pancreatic cancer and comprises a K-Ras Q61H mutation and a TP53, a CDKN2A, a CDKN2B, an MTAP, an SMAD4, an ATM, an AKT2, an RNF43, a GATA6 or an SF381 mutation. In some embodiments, the cancer is multiple myeloma and comprises a K-Ras Q61H mutation and an IGH, a TP53, a CDKN2C, a DNMT3A, an RB1, a TRAF3, an N-Ras, a TET2, an FAF1 or a BRAF mutation. In some embodiments, the cancer is acute myeloid leukemia and comprises a K-Ras Q61H mutation and an N-Ras, a KMT2A, a FLT3, a DNMT3A, a RUNX1, a PTPN11, a TP53, an ETV6, an NPM1 or an MYH11 mutation. In some embodiments, the cancer is melanoma, and the Ras mutation comprises an N-Ras mutation, such as N-Ras Q61R or N-Ras Q61K. In any of the foregoing, a compound may inhibit Ras^{WT} (e.g., K-, H- or N-Ras^{WT}) or Ras^{amp} (e.g., K-, H- or N-Ras^{amp}) as well.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof, is also provided. The cell may be a cancer cell. The cancer cell may be of any type of cancer described herein. The cell may be in vivo or in vitro.

### Combination Therapy

The methods of the invention may include a compound of the invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65: S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this Combination Therapy section, all references are incorporated by reference for the agents described, whether explicitly stated as such or not.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith. A compound of the present invention may be combined with a second, third, or fourth therapeutic agent, or more. A compound of the present invention may be combined with one or more therapeutic agents along with one or more non-drug therapies.

For example, a therapeutic agent may be a steroid. Steroids are known in the art. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. Biologics are known in the art. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. Such checkpoint inhibitors are known in the art. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab). Other anti-TIGIT antibodies are known in the art.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents. Such agents are known in the art.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, Ianiquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, belinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. HER2 inhibitors are known in the art. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. ALK inhibitors are known in the art. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, ERAS-601, SH3809, PF-07284892, or BBP-398), or a pharmaceutically acceptable salt, solvate, isomer (e.g., stereoisomer), prodrug, or tautomer thereof), an SOS1 inhibitor (e.g., BI-1701963, BI-3406, SDR5, BAY-293, MRTX-0902, or RMC-5845, or a pharmaceutically acceptable salt, solvate, isomer (e.g., stereoisomer), prodrug, or tautomer thereof), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, an anti-cancer agent is a SOS1 inhibitor. SOS1 inhibitors are known in the art. In some embodiments, the SOS1 inhibitor is selected from those disclosed in WO 2022219035, WO 2022214594, WO 2022199670, WO 2022146698, WO 2022081912, WO 2022058344, WO 2022026465, WO 2022017519, WO 2021173524, WO 2021130731, WO 2021127429, WO 2021092115, WO 2021105960, WO 2021074227, WO 2020180768, WO 2020180770, WO 2020173935, WO 2020146470, WO 2019201848, WO 2019122129, WO 2018172250, and WO 2018115380, or a pharmaceutically acceptable salt, solvate, isomer (e.g., stereoisomer), prodrug, or tautomer thereof.

In some embodiments, an anti-cancer agent is an additional Ras inhibitor or a Ras vaccine, or another therapeutic modality designed to directly or indirectly decrease the oncogenic activity of Ras. Such agents are known in the art. In some embodiments, an anti-cancer agent is an additional Ras inhibitor. In some embodiments, the Ras inhibitor targets Ras in its active, or GTP-bound state. In some embodiments, the Ras inhibitor targets Ras in its inactive, or GDP-bound state. In some embodiments, the Ras inhibitor is, such as an inhibitor of K-Ras G12C, such as AMG 510, MRTX1257, MRTX849, JNJ-74699157, LY3499446, ARS-1620, ARS-853, BPI-421286, LY3537982, JDQ443, JAB-3312, JAB-21822, JAB-21000, 1B1351, ERAS-3490, RMC-6291, BI 1823911, D-1553, D3S-001, HBI-2438, HS-10370, MK-1084, YL-15293, BBO-8520 (ON/OFF inhibitor), FMC-376 (ON/OFF inhibitor), GEC255, or GDC-6036. In some embodiments, the Ras inhibitor is an inhibitor of K-Ras G12D, such as MRTX1133, JAB-22000, MRTX282, ERAS-4, ERAS-5024, HRS-4642, BI-2852, ASP3082, TH-Z827, TH-7835, RMC-9805, GFH375 (VS-7375), INCB161734 and KD-8. In some embodiments, the Ras inhibitor is a K-Ras G12V inhibitor, such as JAB-23000. In some embodiments, the KRAS(OFF) inhibitor is a pan-KRAS(OFF) inhibitor. In specific embodiments, the pan-KRAS(OFF) inhibitor is JAB-23400, JAB-23425, BI-2493, BI-2865, QTX-3034 (G12D preferring), QTX3544 (G12V preferring), ZG2001, BBO-a, BBO-B, or Pan KRas-IN-1. In some embodiments, the Ras inhibitor is JAB-23400. In some embodiments, the Ras inhibitor is RMC-6236. In some embodiments, the Ras inhibitor is LUNA18. In some embodiments, the Ras inhibitor is BI-2493. In some embodiments, the Ras inhibitor is selected from a Ras(ON) inhibitor (that is, Ras in its GTP-bound state) disclosed in the following, incorporated herein by reference in their entireties, or a pharmaceutically acceptable salt, solvate, isomer (e.g., stereoisomer), prodrug, or tautomer thereof: WO 2022/235870, WO 2022/235864, WO 2022/060836, WO 2021091982, WO 2021091967, WO 2021091956, and WO 2020132597. Other examples of Ras inhibitors are known in the art, such as in the following, incorporated herein by reference in their entireties: WO 2023287896, WO 2023287730, WO 2023284881, WO 2023284730, WO 2023284537, WO 2023283933, WO 2023283213, WO 2023280960, WO 2023280280, WO2023278600, WO 2023280136, WO 2023280026, WO 2023278600, WO 2023274383, WO 2023274324, WO 2023034290, WO 2023020523, WO 2023020521, WO 2023020519, WO 2023020518, WO 2023018812, WO 2023018810, WO 2023018809, WO 2023018699, WO 2023015559, WO 2023014979, WO 2023014006, WO 2023010121, WO 2023009716, WO 2023009572, WO 2023004102, WO 2023003417, WO 2023001141, WO 2023001123, WO 2022271923, WO 2022271823, WO 2022271810, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223.

In some embodiments, a therapeutic agent that may be combined with a compound of the present invention is an inhibitor of the MAP kinase (MAPK) pathway (or “MAPK inhibitor”). Such agents are known in the art. MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. Such agents are known in the art. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist. Such agents are known in the art.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, additional therapeutic agents include FGFR inhibitors, PARP inhibitors, BET inhibitors, PRMT5i inhibitors, MAT2A inhibitors, VEGF inhibitors, and HDAC inhibitors. In some embodiments, a therapeutic agent may be a pan-RTK inhibitor, such as afatinib.

IGF-1R inhibitors are known in the art and include linsitinib, or a pharmaceutically acceptable salt thereof.

EGFR inhibitors are known in the art and include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67: 247-253; Teramoto et al., Cancer 1996, 77: 639-645; Goldstein et al., Clin. Cancer Res. 1995, 1: 1311-1318; Huang et al., 1999, Cancer Res. 15:59(8): 1935-40; and Yang et al., Cancer Res. 1999, 59: 1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics in Oncology Therapeutic Antibody Development, BioTechniques 2005, 39 (4): 565-8; and Paez et al., EGFR Mutations in Lung Cancer Correlation with Clinical Response to Gefitinib Therapy, Science 2004, 304(5676): 1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

MEK inhibitors are known in the art and include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

PI3K inhibitors are known in the art and include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-I-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors are known in the art and include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl): 3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13: 787-97); and triciribine (TCN or API-2 or NCI identifier: NSC 154020; Yang et al., Cancer Res. 2004, 64:4394-9).

mTOR inhibitors are known in the art and include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552, having the structure

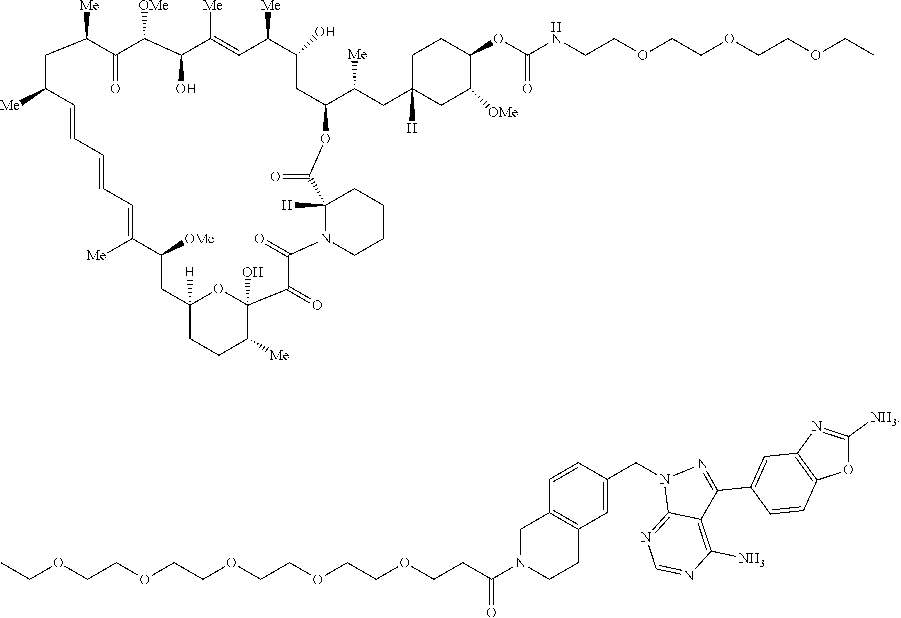

BRAF inhibitors that may be used in combination with compounds of the invention are known in the art and include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E.

MCL-1 inhibitors are known in the art and include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 inhibitors are known in the art. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung, and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2021176072, WO 2021171261, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Ser. No. 11/179,397, U.S. Ser. No. 11/044,675, U.S. Ser. No. 11/034,705, U.S. Ser. No. 11/033,547, U.S. Ser. No. 11/001,561, U.S. Ser. No. 10/988,466, U.S. Ser. No. 10/954,243, U.S. Ser. No. 10/934,302, or U.S. Ser. No. 10/858,359, or a pharmaceutically acceptable salt, solvate, isomer (e.g., stereoisomer), prodrug, or tautomer thereof, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155, having the structure:

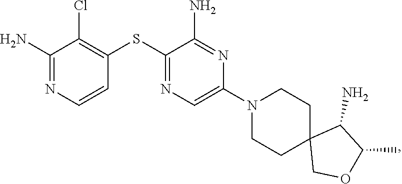

or a pharmaceutically acceptable salt, solvate, isomer (e.g., stereoisomer), prodrug, or tautomer thereof. In some embodiments, the SHP2 inhibitor is RMC-4550. In some embodiments, the SHP2 inhibitor is RMC-4630, having the structure:

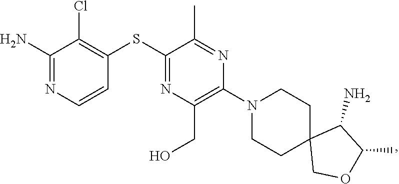 [SMILES: Cc1nc(N2CCC3(CC2)CO[C@@H](C)[C@H]3N)c(CO)nc1Sc1ccnc(N)c1Cl ; ","]

or a pharmaceutically acceptable salt, solvate, isomer (e.g., stereoisomer), prodrug, or tautomer thereof. In some embodiments, the SHP2 inhibitor is JAB-3068, having the structure

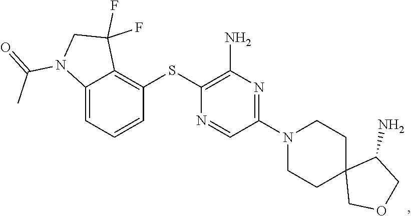 [SMILES: CC(=O)N1CC(F)(F)c2c(Sc3ncc(N4CCC5(CC4)COC[C@H]5N)nc3N)cccc21]

or a pharmaceutically acceptable salt, solvate, isomer (e.g., stereoisomer), prodrug, or tautomer thereof. In some embodiments, the SHP2 inhibitor is JAB-3312. In some embodiments, the SHP2 inhibitor is the following compound,

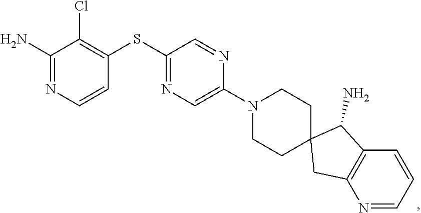 [SMILES: Nc1nccc(Sc2cnc(N3CCC4(CC3)Cc3ncccc3[C@H]4N)cn2)c1Cl]

or a pharmaceutically acceptable salt, solvate, isomer (e.g., stereoisomer), prodrug, or tautomer thereof. In some embodiments, the SHP2 inhibitor is RLY-1971, having the structure

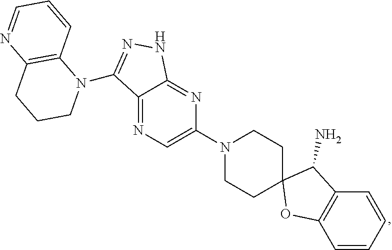 [SMILES: N[C@@H]1c2ccccc2OC12CCN(c1cnc3c(N4CCCc5ncccc54)n[nH]c3n1)CC2]

or a pharmaceutically acceptable salt, solvate, isomer (e.g., stereoisomer), prodrug, or tautomer thereof. In some embodiments, the SHP2 inhibitor is ERAS-601. In some embodiments, the SHP2 inhibitor is BBP-398.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a Ras inhibitor of the present invention is used in combination with a MEK inhibitor and a SOS1 inhibitor. In some embodiments, a Ras inhibitor of the present invention is used in combination with a PD-L1 inhibitor and a SOS1 inhibitor. In some embodiments, a Ras inhibitor of the present invention is used in combination with a PD-L1 inhibitor and a SHP2 inhibitor. In some embodiments, a Ras inhibitor of the present invention is used in combination with a MEK inhibitor and a SHP2 inhibitor. In some embodiments, a Ras inhibitor of the present invention is used in combination with a SHP2 inhibitor and a Ras inhibitor that inhibits multiple Ras isoforms and/or mutants (e.g., RMC-6236). In some embodiments, the cancer is lung cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, the cancer is colorectal cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, a Ras inhibitor of the present invention is used in combination with an immunotherapy, optionally in combination with a chemotherapeutic agent.

Proteasome inhibitors are known in the art and include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents). Other immune therapies are known in the art.

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110 (1): 186-192; Thompson et al., Clin. Cancer Res. 2007, 13 (6): 1757-1761; and WO06/121168 A1), as well as described elsewhere herein.

FGFR inhibitors are known in the art, such as pemigatinib and erdafitinib, including FGFR2 inhibitors and FGFR4 inhibitors. See, e.g., Cancers (Basel), 2021 June; 13(12) 2968.

BET inhibitors are known in the art, such as romidepsin, panobinostat and belinostat. See, e.g., British J. Cancer 124: 1478 (2021).

PRMT5i inhibitors are known in the art, such as PF-0693999, PJ-68 and MRTX1719. See, e.g., Biomed. Pharmacotherapy 144:112252 (2021).

MAT2A inhibitors are known in the art, such as AG-270 and IDE397. See, e.g., Exp Opin Ther Patents (2022) DOI: 10.1080/13543776.2022.2119127.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are known in the art and are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), VEGF inhibitors, EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-Ialfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met. Such agents are known in the art.

Another example of a therapeutic agent that may be used in combination with compounds of the invention is an autophagy inhibitor. Autophagy inhibitors are known in the art and include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent, which are known in the art. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-Ia, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Synthesis of Compound A178—(9S,15S,18S,20aS,21aR)-2-ethyl-18-isopropyl-3-(2-((S)-1-methoxyethyl)pyridin-3-yl)-5,5,19-trimethyl-2,4,5,6,9,10,11,12,15,16,18,19,21,21a,22,23-hexadecahydro-8H-9,13-epimino-1,29-etheno-15,26-methano-24,28-(metheno)cyclopropa[o]pyrrolo[3,4-z][1]oxa[7,10,13,18]tetraazacyclotriacontine-8,14,17,20(20aH)-tetraone

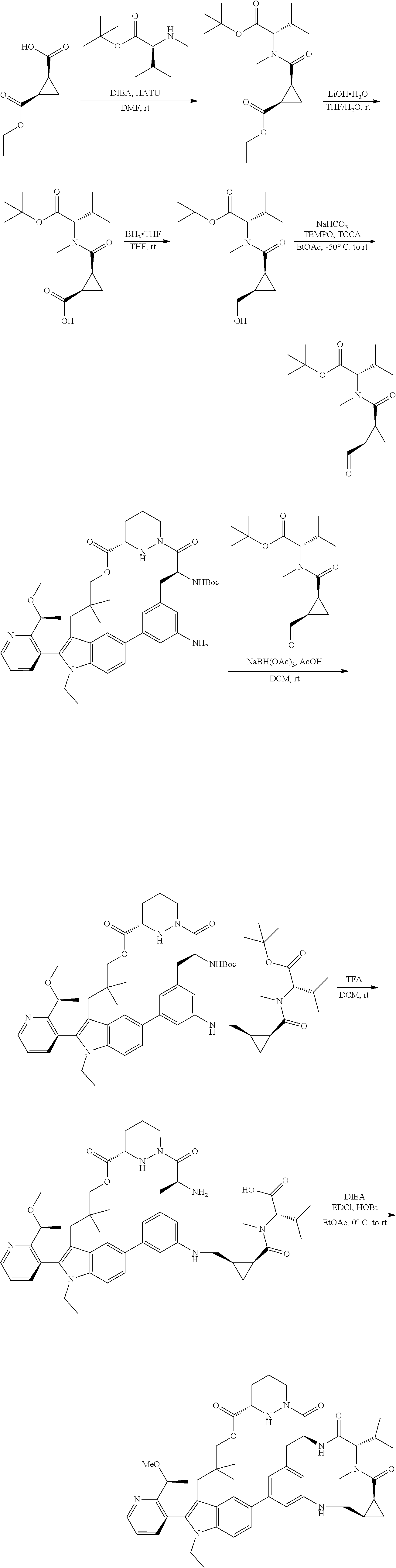

Step 1. To a stirred mixture of (1 S,2R)-2-(ethoxycarbonyl)cyclopropane-1-carboxylic acid (2.70 g, 17.1 mmol, assumed absolute configuration), tert-butyl (2S)-3-methyl-2-(methylamino)butanoate (3.20 g, 17.1 mmol), and DIPEA (6.6 g, 51.2 mmol) in DMF (30 mL) was added HATU (9.70 g, 25.6 mmol) at 0° C. The resulting mixture was stirred for 5 hours at room temperature. The reaction mixture was then diluted with H₂O (40 mL), extracted with DCM (3×30 mL), washed with brine (3×30 mL), dried over anhydrous Na₂SO₄, filtered, concentrated under reduced pressure, and purified by normal phase flash column chromatography to give ethyl (1R,2S)-2-{[(2S)-1-(tert-butoxy)-3-methyl-1-oxobutan-2-yl](methyl)carbamoyl}cyclopropane-1-carboxylate (4.80 g, 77% yield) as a white solid. LCMS (ESI) m/z: [M+H] calcd for C₁₇H₂₉NO₅: 328.2; found 328.2.

Step 2. To a stirred mixture of ethyl (1R,2S)-2-{[(2S)-1-(tert-butoxy)-3-methyl-1-oxobutan-2-yl](methyl)carbamoyl}cyclopropane-1-carboxylate (4.60 g, 14.1 mmol) in THF (50 mL) and H₂O (10 mL) was added LiOH·H₂O (1.20 g, 28.1 mmol) at 0° C. The resulting mixture was stirred for 5 hours at room temperature. The reaction mixture was then concentrated under reduced pressure, diluted with H₂O (20 mL), adjusted to pH 6 by the addition of aqueous HCl, extracted with EtOAc (2×20 mL), and concentrated under reduced pressure to give (1R,2S)-2-{[(2S)-1-(tert-butoxy)-3-methyl-1-oxobutan-2-yl](methyl)carbamoyl}cyclopropane-1-carboxylic acid (3.40 g, crude) as a dark oil. This material was taken directly to the next reaction without further purification. LCMS (ESI) m/z: [M+H] calcd for C₁₅H₂₅NO₅: 300.2; found 300.2.

Step 3. To a stirred mixture of (1R,2S)-2-{[(2S)-1-(tert-butoxy)-3-methyl-1-oxobutan-2-yl](methyl)carbamoyl}cyclopropane-1-carboxylic acid (3.00 g, crude) in THF (30 mL) was added BH₃·THF (5 mL) at 0° C. The resulting mixture was stirred for 16 hours at room temperature. The reaction mixture was then quenched by the addition of sat. aq. NH₄Cl (10 mL), extracted with EtOAc (2×20 mL), washed with brine (2×20 mL), dried over anhydrous Na₂SO₄, filtered, and concentrated under reduced pressure to give tert-butyl (2S)-2-{1-[(1 S,2R)-2-(hydroxymethyl) cyclopropyl]-N-methylformamido}-3-methylbutanoate (1.70 g, crude) as a dark oil. This material was taken directly to the next reaction without further purification. LCMS (ESI) m/z: [M+H] calcd for C₁₅H₂₇NO₄: 286.2; found 286.3.

Step 4. To a stirred mixture of tert-butyl (2S)-2-{1-[(1 S,2R)-2-(hydroxymethyl)cyclopropyl]-N-methylformamido}-3-methylbutanoate (850 mg, crude), NaHCO₃ (751 mg, 8.9 mmol) and TEMPO (9.3 mg, 0.06 mmol) in EtOAc (20 mL) was added a solution of TCCA (727 mg, 3.1 mmol) in EtOAc (10 mL) dropwise at −50° C. The resulting mixture was stirred at 0° C. for 2 hours and was then stirred at room temperature for 13 hours. The reaction mixture was quenched by the addition of sat. aq. Na₂S₂O₃ (10 mL), filtered, concentrated under reduced pressure, and purified by normal phase flash column chromatography to afford tert-butyl (2S)-2-{1-[(1 S,2R)-2-formylcyclopropyl]-N-methylformamido}-3-methylbutanoate (400 mg, 23% yield over 3 steps) as a brown oil. LCMS (ESI) m/z: [M+H] calcd for C₁₅H₂₅NO₄: 284.2; found 284.2.

Step 5. To a stirred mixture of tert-butyl ((6³S,4S)-2⁵-amino-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (300 mg, 0.40 mmol) and tert-butyl (2S)-2-{1-[(1S,2R)-2-formylcyclopropyl]-N-methylformamido}-3-methylbutanoate (139 mg, 0.50 mmol) in DCM (10 mL) were added NaBH(OAc)₃ (174 mg, 0.80 mmol) and AcOH (12.0 mg, 0.20 mmol) at room temperature. The resulting mixture was stirred for 5 hours at room temperature. The reaction mixture was then quenched by the addition of H₂O (10 mL), extracted with DCM (2×20 mL), washed with brine (2×20 mL), dried over anhydrous Na₂SO₄, filtered, concentrated under reduced pressure, and purified by normal phase flash column chromatography to afford tert-butyl N-((1 S,2R)-2-((((6³S,4S)-4-((tert-butoxycarbonyl)amino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)methyl)cyclopropane-1-carbonyl)-N-methyl-L-valinate (240 mg, 52% yield) as a white solid. LCMS (ESI) m/z: [M+H] calcd for C₅₇H₇₉N₇O₉: 1006.6; found 1006.3.

Step 6. To a stirred mixture tert-butyl N-((1 S,2R)-2-((((6³S,4S)-4-((tert-butoxycarbonyl)amino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)methyl)cyclopropane-1-carbonyl)-N-methyl-L-valinate (200 mg, 0.20 mmol) in DCM (5 mL) was added TFA (1.0 mL). The resulting mixture was stirred for 2 hours at room temperature and was then concentrated under reduced pressure to give N-((1 S,2R)-2-((((6³S,4S)-4-amino-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)methyl)cyclopropane-1-carbonyl)-N-methyl-L-valine (200 mg, crude) as a solid. This material was taken directly to the next reaction without further purification. LCMS (ESI) m/z: [M+H] calcd for C₄₈H₆₃N₇O₇: 850.5; found 850.4.

Step 7. To a stirred mixture of N-((1 S,2R)-2-((((6³S,4S)-4-amino-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)methyl)cyclopropane-1-carbonyl)-N-methyl-L-valine (230 mg, crude), DIPEA (349 mg, 2.70 mmol) and EDCI (259 mg, 1.40 mmol) in DCM (20 mL) was added HOBt (182 mg, 1.4 mmol) at 0° C. The resulting mixture was stirred for 6 hours at room temperature, concentrated under reduced pressure, and purified by reversed-phase prep-HPLC to afford (9S,15S,18S,20aS,21aR)-2-ethyl-18-isopropyl-3-(2-((S)-1-methoxyethyl)pyridin-3-yl)-5,5,19-trimethyl-2,4,5,6,9,10,11,12,15,16,18,19,21,21a,22,23-hexadecahydro-8H-9,13-epimino-1,29-etheno-15,26-methano-24,28-(metheno)cyclopropa[o]pyrrolo[3,4-z][1]oxa[7,10,13,18]tetraazacyclotriacontine-8,14,17,20(20aH)-tetraone (2.8 mg, 1.5% yield over 2 steps) as a white solid. LCMS (ESI) m/z: [M+H] calcd for C₄₈H₆₁N₇O₆: 832.5; found 832.5; ¹H NMR (400 MHz, CD₃OD) δ 8.74-8.72 (m, 1H), 8.11 (s, 1H), 7.87-7.85 (m, 1H), 7.69-7.67 (m, 2H), 7.55-7.52 (m, 1H), 7.49-7.47 (m, 1H), 7.11 (s, 1H), 6.93 (s, 1H), 6.62 (s, 1H), 5.62-5.60 (m, 1H), 5.06-5.03 (m, 1H), 4.62-4.60 (m, 1H), 4.45-4.42 (m, 1H), 4.34-4.32 (m, 1H), 4.18-4.11 (m, 1H), 4.08-4.02 (m, 2H), 3.87-3.85 (m, 1H), 3.78-3.76 (m, 1H), 3.48-3.40 (m, 2H), 3.27 (s, 3H), 3.13-3.11 (m, 1H), 3.08 (s, 3H), 2.87-2.82 (m, 3H), 2.73-2.70 (m, 2H), 2.19-2.16 (m, 2H), 2.03-1.94 (m, 2H), 1.69-1.67 (m, 2H), 1.47-1.45 (m, 3H), 1.30 (s, 3H), 1.01-0.99 (m, 3H), 0.97-0.96 (m, 2H), 0.89-0.87 (m, 2H), 0.86 (s, 1H), 0.58 (s, 3H).

### Synthesis of Compound A175—(9S,15S,18S,20aS,21aS,E)-2-ethyl-18-isopropyl-3-(2-((S)-1-methoxyethyl)pyridin-3-yl)-5,5,19-trimethyl-2,4,5,6,9,10,11,12,15,16,18,19,21,21a-tetradecahydro-8H-9,13-epimino-1,29-etheno-15,26-methano-24,28-(metheno)cyclopropa[o]pyrrolo[3,4-z][1]oxa[7,10,13]triazacyclotriacontine-8,14,17,20(20aH)-tetraone

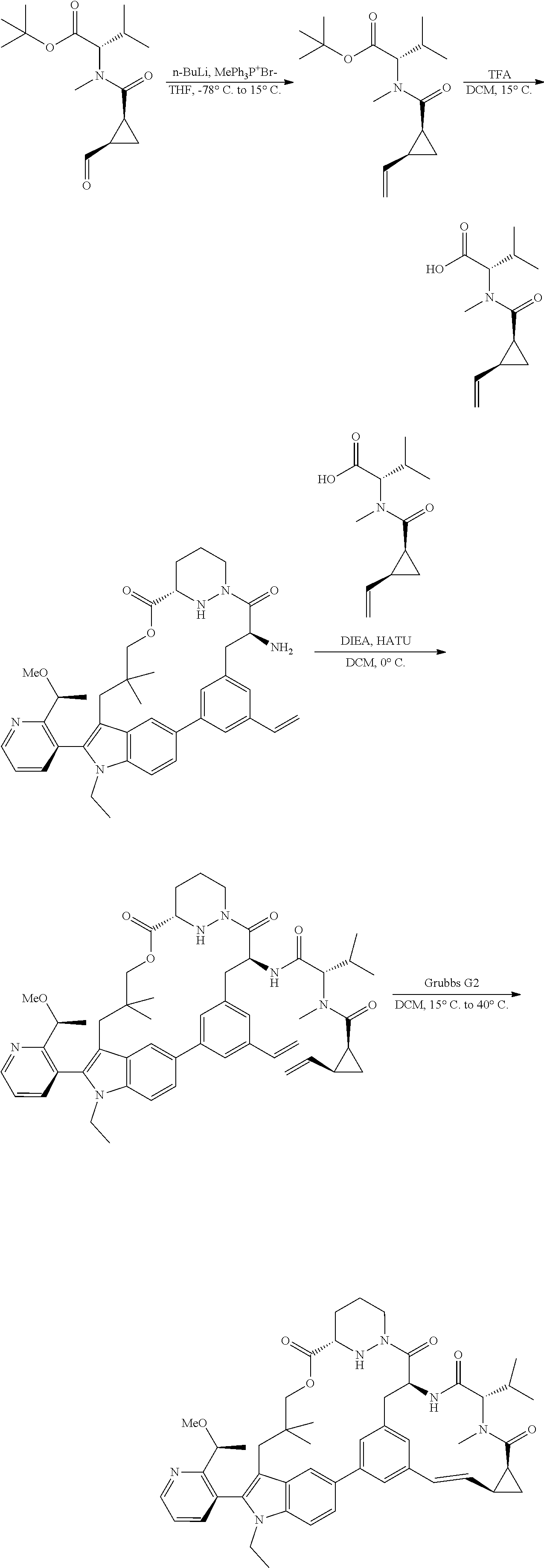

Step 1. To a stirred solution of methyltriphenylphosphonium bromide (3.80 g, 10.6 mmol) in dry THF (70 mL) was added n-BuLi (2.4 M in hexane, 4.4 mL, 10.6 mmol) dropwise at −78° C. This mixture was stirred at −78° C. for 30 minutes and was then stirred at 10° C. for 1 hour. After this time, a solution of tert-butyl N-((1 S,2R)-2-formylcyclopropane-1-carbonyl)-N-methyl-L-valinate (600 mg, 2.12 mmol, assumed absolute configuration) in dry THF (10 mL) was added dropwise at 15° C., and the resulting mixture was stirred at 15° C. for 16 hours. The reaction mixture was quenched with 10% aq. NH₄Cl (20 mL), extracted with EtOAc, (2×100 mL), concentrated under reduced pressure, and purified by normal phase flash column chromatograph to afford tert-butyl N-methyl-N-((1S,2S)-2-vinylcyclopropane-1-carbonyl)-L-valinate (500 mg, 75% yield) as an oil. LCMS (ESI) m/z: [M+H] calcd for C₁₆H₂₇NO₃: 282.2; found 282.3.

Step 2. To a solution of tert-butyl N-methyl-N-((1 S,2S)-2-vinylcyclopropane-1-carbonyl)-L-valinate (120 mg, 0.43 mmol) in DCM (5 mL) was added TFA (2.5 mL) at 15° C. The resulting mixture was stirred at 15° C. for 1 hour and was then concentrated under reduced pressure to afford N-methyl-N-((1 S,2S)-2-vinylcyclopropane-1-carbonyl)-L-valine (100 mg, crude) as an oil. This material was taken directly to the next reaction without further purification. LCMS (ESI) m/z: [M+H] calcd for C₁₂H₁₉NO₃: 226.1; found 226.2.

Step 3. To a stirred solution of (6³S,4S)-4-amino-1¹-ethyl-1²-(2-((S)-1-methoxyethyl)pyridin-3-yl)-10,10-dimethyl-2⁵-vinyl-6¹,6²,6³,6⁴,6⁵,6⁶-hexahydro-1¹H-8-oxa-1(5,3)-indola-6(1,3)-pyridazina-2(1,3)- benzenacycloundecaphane-5,7-dione (120 mg, 0.18 mmol) in DMF (5 mL) were added N-methyl-N-((1 S,2S)-2-vinylcyclopropane-1-carbonyl)-L-valine (81.0 mg, crude), DIEA (232 mg, 1.80 mmol), and HATU (102 mg, 0.27 mmol) in portions at 0° C. The resulting mixture was stirred at 0° C. for 1 hour. The reaction mixture was then poured into cold H₂O (30 mL), extracted with EtOAc (2×30 mL), washed with H₂O (20 mL), treated with brine (20 mL), concentrated under reduced pressure, and purified by normal phase flash column chromatography to afford (1 S,2S)—N-((2S)-1-(((6³S,4S)-1¹-ethyl-1²-(2-((S)-1-methoxyethyl)pyridin-3-yl)-10,10-dimethyl-5,7-dioxo-2⁵-vinyl-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-2-vinylcyclopropane-1-carboxamide (110 mg, 71% yield) as white solid. LCMS (ESI) m/z: [M+H] calcd for C₅₁H₆₄N₆O₆: 857.5; found 857.3.

Step 4. To a solution of (1 S,2S)—N-((2S)-1-(((6³S,4S)-1¹-ethyl-1²-(2-((S)-1-methoxyethyl)pyridin-3-yl)-10,10-dimethyl-5,7-dioxo-2⁵-vinyl-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-2-vinylcyclopropane-1-carboxamide (100 mg, 0.12 mmol) stirred in DCM (10 mL) was added 2^{nd} generation Grubbs catalyst (51 mg, 0.060 mmol) at 15° C. under an atmosphere of N₂. This resulting mixture was stirred at 40° C. for 16 hours under an atmosphere of N₂. The reaction mixture was then concentrated under reduced pressure and was purified by normal phase prep-TLC to afford (9S,15S,18S,20aS,21aS,E)-2-ethyl-18-isopropyl-3-(2-((S)-1-methoxyethyl)pyridin-3-yl)-5,5,19-trimethyl-2,4,5,6,9,10,11,12,15,16,18,19,21,21a-tetradecahydro-8H-9,13-epimino-1,29-etheno-15,26-methano-24,28-(metheno)cyclopropa[o]pyrrolo[3,4-z][1]oxa[7,10,13]triazacyclotriacontine-8,14,17,20(20aH)-tetraone (12.0 mg, 11% yield) as a white solid. LCMS (ESI) m/z: [M+H] calcd for C₄₉H₆₀N₆O₆: 829.4; found 829.3; ¹H NMR (400 MHz, MeOD) δ 8.78-8.69 (m, 1H), 8.51 (s, 1H), 8.13 (s, 1H), 7.82 (dd, J=7.7, 1.4 Hz, 1H), 7.68 (d, J=7.8 Hz, 1H), 7.61 (s, 1H), 7.57-7.49 (m, 2H), 7.46 (s, 1H), 7.28 (s, 1H), 6.73 (d, J=15.7 Hz, 1H), 5.69 (dd, J=15.7, 10.6 Hz, 1H), 5.51 (d, J=9.7 Hz, 1H), 4.92 (d, J=11.2 Hz, 1H), 4.82 (d, J=12.5 Hz, 1H), 4.43 (d, J=11.5 Hz, 1H), 4.37 (dd, J=12.3, 6.2 Hz, 1H), 4.32-4.27 (m, 1H), 4.24 (d, J=12.2 Hz, 1H), 4.19-4.08 (m, 1H), 3.94 (d, J=10.9 Hz, 1H), 3.76 (d, J=11.0 Hz, 1H), 3.37 (s, 2H), 3.21 (d, J=14.6 Hz, 1H), 3.11 (s, 3H), 3.07-2.98 (m, 1H), 2.82-2.71 (m, 2H), 2.48 (d, J=14.6 Hz, 1H), 2.37 (dd, J=14.8, 7.8 Hz, 1H), 2.30-2.12 (m, 3H), 1.96 (d, J=13.0 Hz, 1H), 1.85 (d, J=12.6 Hz, 1H), 1.63 (dd, J=16.6, 8.2 Hz, 1H), 1.53 (dd, J=11.1, 5.5 Hz, 1H), 1.44 (d, J=6.1 Hz, 3H), 1.31 (dd, J=12.7, 7.5 Hz, 1H), 0.92 (dt, J=6.4, 4.8 Hz, 12H), 0.44 (s, 3H).

### Synthesis of Compound A152—(9S,15S,18R)-2-ethyl-18-isopropyl-3-(2-((S)-1-methoxyethyl)-5-(4-methylpiperazin-1-yl)pyridin-3-yl)-5,5-dimethyl-2,4,5,6,9,10,11,12,15,16,18,19,21,22-tetradecahydro-8H-9,13-epimino-1,29-etheno-15,26-methano-24,28-(metheno)pyrrolo[3,4-u][1,4,17]trioxa[8,11]diazacyclononacosine-8,14,17-trione

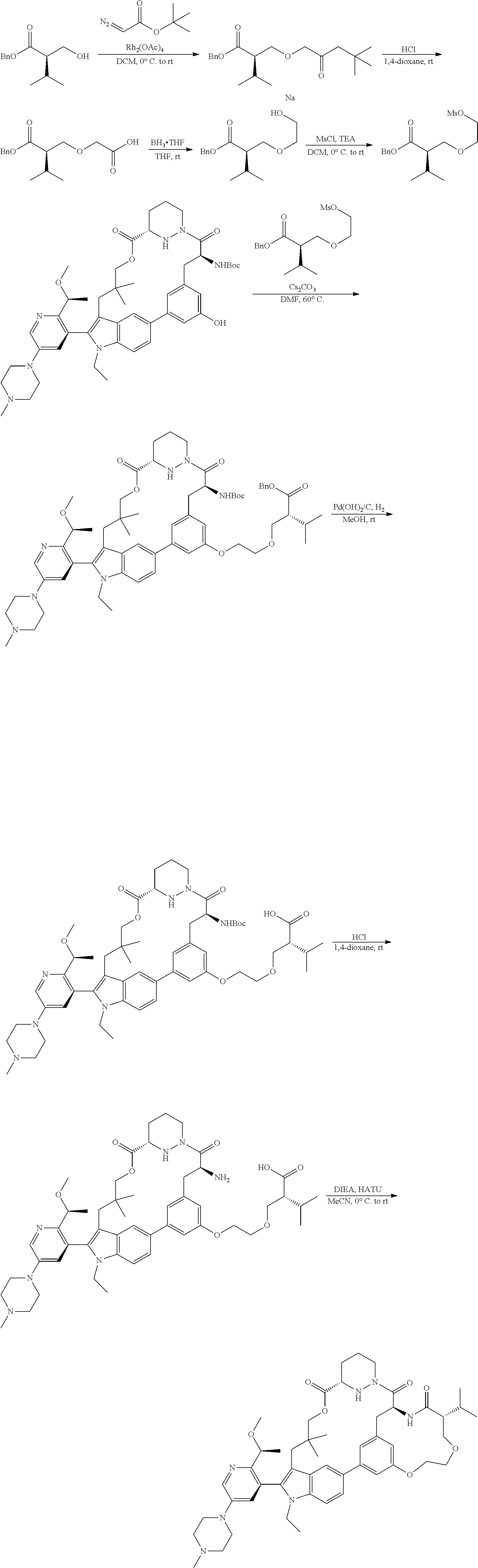

Step 1. To a stirred solution of benzyl (2R)-2-(hydroxymethyl)-3-methylbutanoate (10.0 g, 45.0 mmol) and tert-butyl 2-diazoacetate (12.8 g, 90.0 mmol) in DCM (100 mL) was added Rh₂(OAc)₄ (994 mg, 2.25 mmol) in portions at 0° C. The resulting mixture was stirred for 16 hours at room temperature. The reaction mixture was filtered through Celite®, the filter cake washed with DCM (3×20 mL), the filtrate concentrated under reduced pressure, and the residue purified by normal phase flash column chromatography to afford benzyl (2R)-2-[[2-(tert-butoxy)-2-oxoethoxy]methyl]-3-methylbutanoate (6.00 g, 40% yield) as a colorless oil. LCMS (ESI) m/z: [M+Na] calcd for C₁₉H₂₈O₅: 359.2; found 359.2.

Step 2. Benzyl (2R)-2-[[2-(tert-butoxy)-2-oxoethoxy]methyl]-3-methylbutanoate (6.00 g, 17.8 mmol) was added to a stirred solution of HCl in 1,4-dioxane (4.0 M, 15.0 mL, 60.0 mmol) at 0° C. The resulting mixture was stirred for 2 hours at room temperature. The reaction mixture was then concentrated under reduced pressure, neutralized to pH 7 by the addition of sat. aq. NaHCO₃, extracted with EtOAc (3×20 mL), washed with brine (3×5 mL), dried over anhydrous Na₂SO₄, filtered, and concentrated under reduced pressure to afford [(2R)-3-(benzyloxy)-2-isopropyl-3-oxopropoxy]acetic acid (3.00 g, crude) as a light yellow oil. This material was taken directly to the next reaction without further purification. LCMS (ESI) m/z: [M+Na] calcd for C₁₅H₂₀O₅: 303.1; found 303.1.

Step 3. [(2R)-3-(benzyloxy)-2-isopropyl-3-oxopropoxy]acetic acid (3.00 g, crude) was added to a stirred solution of BH₃·THF complex (1.0 M in THF, 32.1 mL, 32.1 mmol) at 0° C. The resulting mixture was stirred for 2 hours at room temperature and was then concentrated under reduced pressure to afford benzyl (2R)-2-[(2-hydroxyethoxy) methyl]-3-methylbutanoate (1.50 g, crude) as a light-yellow oil. This material was taken directly to the next reaction without further purification. LCMS (ESI) m/z: [M+H] calcd for C₁₅H₂₂O₄: 267.2; found 267.2.

Step 4. To a stirred solution of benzyl (2R)-2-[(2-hydroxyethoxy)methyl]-3-methylbutanoate (1.00 g, crude) and TEA (1.14 g, 11.3 mmol) in DCM (10 mL) was added MsCl (860 mg, 7.51 mmol) in portions at 0° C. The resulting mixture was stirred for 1 hour at room temperature. The reaction mixture was then concentrated under reduced pressure and was purified by normal phase flash column chromatography to afford benzyl (2R)-2-[[2-(methanesulfonyloxy)ethoxy]methyl]-3-methylbutanoate (687 mg, 17% yield over 3 steps) as a colorless oil. LCMS (ESI) m/z: [M+H] calcd for C₁₆H₂₄O₆S: 345.1; found 345.2.

Step 5. To a stirred solution of tert-butyl ((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.10 g, 1.31 mmol) and Cs₂CO₃ (1.28 g, 3.94 mmol) in DMF (10 mL) was added benzyl (2R)-2-[[2-(methanesulfonyloxy)ethoxy]methyl]-3-methylbutanoate (678 mg, 1.97 mmol) in portions at room temperature under an atmosphere of N₂. The resulting mixture was stirred for 2 hours at 60° C. The reaction mixture was then diluted with EtOAc, washed with H₂O, treated with brine, concentrated under reduced pressure, and purified by reversed-phase flash column chromatography to afford benzyl (2R)-2-((2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)ethoxy)methyl)-3-methylbutanoate (750 mg, 53% yield) as a white solid. LCMS (ESI) m/z: [M+H] calcd for C₆₂H₈₃N₇O₁₀: 1086.6; found 1087.3.

Step 6. To a stirred solution of benzyl (2R)-2-((2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)ethoxy)methyl)-3-methylbutanoate (840 mg, 0.773 mmol) in MeOH (5.0 mL) was added Pd(OH)₂/C (724 mg, 20% wt.) under at atmosphere of N₂. The resulting mixture was stirred at room temperature for 2 hours under an atmosphere of H₂. The reaction mixture was then filtered through Celite® and the filtrate was concentrated under reduced pressure to afford (2R)-2-((2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)ethoxy)methyl)-3-methylbutanoic acid (670 mg, crude) as a white solid. This material was taken directly to the next reaction without further purification. LCMS (ESI) m/z: [M+H] calcd for C₅₅H₇₇N₇O₁₀: 996.6; found 996.4.

Step 7. (2R)-2-((2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)ethoxy)methyl)-3-methylbutanoic acid (670 mg, crude) was added to a solution of HCl in 1,4-dioxane (4.0 M, 5.0 mL, 20 mmol) at 0° C. The resulting mixture was stirred for 1 h at room temperature. The reaction mixture was then concentrated under reduced pressure to afford product (2R)-2-((2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)ethoxy)methyl)-3-methylbutanoic acid (810 mg, crude) as a yellow solid. This material was taken directly to the next reaction without further purification. LCMS (ESI) m/z: [M+H] calcd for C₅₀H₆₉N₇O₈: 896.5; found 896.9.

Step 8. To a stirred solution of (2R)-2-((2-(((6³S,4S)-4-amino-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)ethoxy)methyl)-3-methylbutanoic acid (810 mg, crude) and DIEA (3.50 g, 27.1 mmol) in MeCN (800 mL) was added HATU (1.03 g, 2.71 mmol) in portions at 0° C. The resulting mixture was stirred for 2 hours at room temperature. The reaction mixture was then concentrated under reduced pressure and the resulting residue was purified by reversed-phase prep-HPLC to afford (9S,15S,18R)-2-ethyl-18-isopropyl-3-(2-((S)-1-methoxyethyl)-5-(4-methylpiperazin-1-yl)pyridin-3-yl)-5,5-dimethyl-2,4,5,6,9,10,11,12,15,16,18,19,21,22-tetradecahydro-8H-9,13-epimino-1,29-etheno-15,26-methano-24,28-(metheno)pyrrolo[3,4-u][1,4,17]trioxa[8,11]diazacyclononacosine-8,14,17-trione (76.0 mg, 11% yield over 3 steps) as a white solid. LCMS (ESI) m/z: [M+H] calcd for C₅₀H₆₇N₇O₇: 878.5; found 878.6; ¹H NMR (400 MHz, DMSO-d₆) δ 8.46 (d, J=2.8 Hz, 1H), 8.37 (d, J=9.8 Hz, 1H), 8.05 (s, 1H), 7.67 (d, J=8.5 Hz, 1H), 7.57 (d, J=8.5 Hz, 1H), 7.48 (s, 1H), 7.26 (s, 1H), 7.21-6.96 (m, 2H), 5.54 (t, J=9.4 Hz, 1H), 5.16 (d, J=12.3 Hz, 1H), 4.31-4.22 (m, 4H), 4.21-3.91 (m, 3H), 3.77 (d, J=11.0 Hz, 2H), 3.65-3.39 (m, 4H), 3.26 (s, 4H), 3.21 (s, 3H), 3.03 (d, J=14.5 Hz, 1H), 2.86-2.63 (m, 3H), 2.48-2.40 (m, 7H), 2.22 (s, 3H), 2.07 (br s, 1H), 1.90-1.72 (m, 2H), 1.69-1.45 (m, 2H), 1.33 (d, J=6.1 Hz, 3H), 1.10-0.93 (m, 6H), 0.89 (d, J=6.5 Hz, 3H) 0.80 (d, J=6.5 Hz, 3H), 0.38 (s, 3H).

### Synthesis of Compound A285—(9S,15S,18S,21S)-3-(5-(4-cyclopropylpiperazin-1-yl)-2-((S)-1-methoxyethyl)pyridin-3-yl)-2-ethyl-18-isopropyl-5,5-dimethyl-21-(4-methylpiperazin-1-yl)-2,4,5,6,9,10,11,12,15,16,21,22-dodecahydro-8H,14H,20H-9,13-epimino-1,29-etheno-15,26-methano-24,28-(metheno)pyrrolo[3,4-q][1,13,26]trioxa[4,7]diazacyclononacosine-8,14,17(18H)-trione

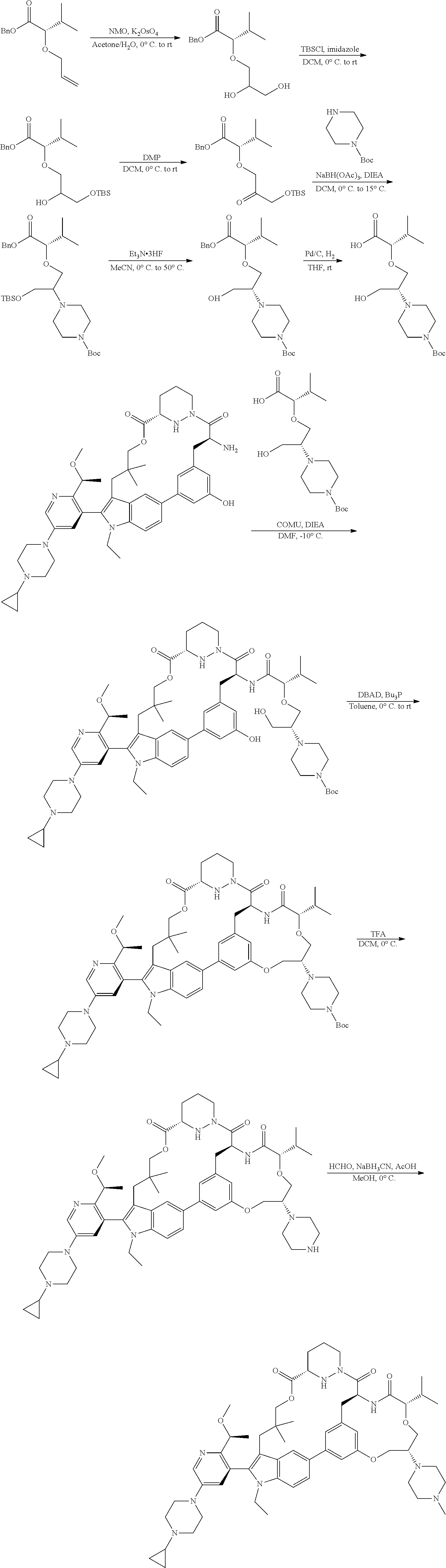

Step 1. To a stirred solution of benzyl (2S)-3-methyl-2-(prop-2-en-1-yloxy)butanoate (110 g, 443 mmol) and 4-methylmorpholine N-oxide (104 g, 886 mmol) in acetone (500 mL) and H₂O (500 mL) was added potassium osmate (VI) dihydrate (4.90 g, 13.3 mmol) in portions at 0° C. under an atmosphere of argon. The resulting mixture was stirred at room temperature for 16 hours. The reaction mixture was then extracted with EtOAc (3×1 L), washed with brine (3×1 L), dried over anhydrous Na₂SO₄, filtered, concentrated under reduced pressure, and purified by normal phase flash column chromatography to afford benzyl (2S)-2-(2,3-dihydroxypropoxy)-3-methylbutanoate (95.0 g, 76% yield) as a clear oil. LCMS (ESI) m/z: [M+H] calcd for C₁₅H₂₂O₅: 283.2; found 283.1.

Step 2. To a stirred solution of benzyl (2S)-2-(2,3-dihydroxypropoxy)-3-methylbutanoate (4.00 g, 14.2 mmol) and imidazole (1.93 g, 28.3 mmol) in DCM (40 mL) was added TBSCI (2.03 g, 13.5 mmol) dropwise at 0° C. under an atmosphere of argon. The resulting mixture was stirred at room temperature for 16 hours. The reaction mixture was then diluted with H₂O (50 mL), extracted with EtOAc (3×60 mL), washed with brine (3×80 mL), dried over anhydrous Na₂SO₄, filtered, concentrated under reduced pressure, and purified by reversed-phase flash column chromatography to afford benzyl (2S)-2-{3-[(tert-butyldimethylsilyl)oxy]-2-hydroxypropoxy}-3-methylbutanoate (3.70 g, 66% yield) as a yellow oil. LCMS (ESI) m/z: [M+H] calcd for C₂₁H₃₆O₅Si: 397.2; found 397.1.

Step 3. To a stirred solution of benzyl (2S)-2-{3-[(tert-butyldimethylsilyl)oxy]-2-hydroxypropoxy}-3-methylbutanoate (2.00 g, 5.04 mmol) in DCM (20 mL) was added Dess-Martin periodinane (4.28 g, 10.1 mmol) dropwise at 0° C. under an atmosphere of argon. The resulting mixture was stirred at room temperature for 5 hours. The reaction mixture was then diluted with sat. aq. NaHCO₃ (20 mL), extracted with EtOAc (3×30 mL), washed with brine (3×50 mL), dried over anhydrous Na₂SO₄, filtered, and concentrated under reduced pressure to give benzyl (S)-2-(3-((tert-butyldimethylsilyl)oxy)-2-oxopropoxy)-3-methylbutanoate (1.95 g, crude). This material was taken directly to the next reaction without further purification. LCMS (ESI) m/z: [M+NH₄] calcd for C₂₁H₃₄O₅Si: 412.3; found 412.2.

Step 4. To a stirred solution of benzyl (S)-2-(3-((tert-butyldimethylsilyl)oxy)-2-oxopropoxy)-3-methylbutanoate (1.95 g, crude) and tert-butyl piperazine-1-carboxylate (920 mg, 4.94 mmol) in DCM (20 mL) were added DIEA (640 mg, 4.94 mmol) and NaBH(OAc)₃ (1.05 g, 4.94 mmol) in portions at 0° C. under an atmosphere of argon. The resulting mixture was stirred at 15° C. for 5 hours. The reaction was quenched with sat. aq. NH₄Cl at 0° C., extracted with EtOAc (3×60 mL), washed with brine (3×70 mL), dried over anhydrous Na₂SO₄, filtered, concentrated under reduced pressure, and purified by reversed-phase flash column chromatography to afford tert-butyl 4-((4S)-4-isopropyl-10,10,11,11-tetramethyl-3-oxo-1-phenyl-2,5,9-trioxa-10-siladodecan-7-yl)piperazine-1-carboxylate (1.00 g, 35% yield over 2 steps) as a yellow oil. LCMS (ESI) m/z: [M+H] calcd for C₃₀H₅₂N₂O₆Si: 565.4; found 565.3.

Step 5. To a stirred solution tert-butyl 4-((4S)-4-isopropyl-10,10,11,11-tetramethyl-3-oxo-1-phenyl-2,5,9-trioxa-10-siladodecan-7-yl)piperazine-1-carboxylate (1.00 g, 1.77 mmol) in MeCN (5.0 mL) was added Et₃N·3HF (5.00 mL, 30.7 mmol) dropwise at 0° C. under an atmosphere of argon. The resulting mixture was stirred at 50° C. for 2 hours. The reaction mixture was then basified to pH 7 by the addition of sat. aq. NaHCO₃, extracted with EtOAc (3×30 mL), washed with brine (3×70 mL), dried over anhydrous Na₂SO₄, filtered, concentrated under reduced pressure, and purified by reversed-phase flash column chromatography to afford tert-butyl 4-(1-(((S)-1-(benzyloxy)-3-methyl-1-oxobutan-2-yl)oxy)-3-hydroxypropan-2-yl)piperazine-1-carboxylate (600 mg) as a yellow oil. This mixture of epimers was then resolved by chiral prep-SFC to afford tert-butyl 4-((R)-1-(((S)-1-(benzyloxy)-3-methyl-1-oxobutan-2-yl)oxy)-3-hydroxypropan-2-yl)piperazine-1-carboxylate (110 mg, 18% yield, assumed absolute configuration) as a yellow oil and tert-butyl 4-((S)-1-(((S)-1-(benzyloxy)-3-methyl-1-oxobutan-2-yl)oxy)-3-hydroxypropan-2-yl)piperazine-1-carboxylate (380 mg, 64% yield, assumed absolute configuration) as a yellow oil. LCMS (ESI) m/z: [M+H] calcd for C₂₄H₃₈N₂O₆: 451.3; found 451.3.

Step 6. A solution of tert-butyl 4-((R)-1-(((S)-1-(benzyloxy)-3-methyl-1-oxobutan-2-yl)oxy)-3-hydroxypropan-2-yl)piperazine-1-carboxylate (110 mg, 0.244 mmol) and Pd/C (100 mg) in THF (20 mL) was stirred at room temperature for 5 hours under an atmosphere of H₂. The resulting mixture was then filtered, the filter cake washed with MeOH (5×10 mL), and the filtrate concentrated under reduced pressure to give (S)-2-((R)-2-(4-(tert-butoxycarbonyl)piperazin-1-yl)-3-hydroxypropoxy)-3-methylbutanoic acid (70 mg, crude). This material was taken directly to the next reaction without further purification. LCMS (ESI) m/z: [M+H] calcd for C₁₇H₃₂N₂O₆: 361.2; found 361.1.

Step 7. To a stirred solution of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (130 mg, 0.170 mmol) and (S)-2-((R)-2-(4-(tert-butoxycarbonyl)piperazin-1-yl)-3-hydroxypropoxy)-3-methylbutanoic acid (67.5 mg, crude) in DMF (1.5 mL) were added DIEA (220 mg, 1.70 mmol) and a solution of COMU (78.7 mg, 0.184 mmol) in DMF (0.2 mL) dropwise at −15° C. under an atmosphere of argon. The resulting mixture was stirred at −10° C. for 1 hour. The reaction mixture was then diluted with H₂O (30 mL), extracted with EtOAc (3×20 mL), washed with brine (3×50 mL), dried over anhydrous Na₂SO₄, filtered, concentrated under reduced pressure, and purified by normal phase flash column chromatography to afford tert-butyl 4-((2R)-1-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)oxy)-3-hydroxypropan-2-yl)piperazine-1-carboxylate (150 mg, 80% yield) as a white solid. LCMS (ESI) m/z: [M+H] calcd for C₆₁H₈₇N₉O₁₀: 1106.7; found 1106.7.

Step 8. To a stirred solution of tert-butyl 4-((2R)-1-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)oxy)-3-hydroxypropan-2-yl)piperazine-1-carboxylate (150 mg, 0.137 mmol) and DBAD (158 mg, 0.685 mmol) in toluene (1.5 mL) was added tributylphosphine (133 mg, 0.685 mmol) dropwise at 0° C. under an atmosphere of argon. The resulting mixture was stirred at room temperature for 16 hours. The reaction mixture was then diluted with H₂O (10 mL), extracted with EtOAc (3×30 mL), washed with brine (3×70 mL), dried over anhydrous Na₂SO₄, filtered, concentrated under reduced pressure, and purified by reversed-phase flash column chromatography to afford tert-butyl 4-((9S,15S,18S,21 S)-3-(5-(4-cyclopropylpiperazin-1-yl)-2-((S)-1-methoxyethyl)pyridin-3-yl)-2-ethyl-18-isopropyl-5,5-dimethyl-8,14,17-trioxo-2,4,5,6,9,10,11,12,15,16,17,18,21,22-tetradecahydro-8H,14H,20H-9,13-epimino-1,29-etheno-15,26-methano-24,28-(metheno)pyrrolo[3,4-q][1,13,26]trioxa[4,7]diazacyclononacosin-21-yl)piperazine-1-carboxylate (60 mg, 40% yield) as a white solid. LCMS (ESI) m/z: [M+H] calcd for C₆₁H₈₅N₉O₉: 1088.7; found 1088.6.

Step 9. To a stirred solution of tert-butyl 4-((9S,15S,18S,21 S)-3-(5-(4-cyclopropylpiperazin-1-yl)-2-((S)-1-methoxyethyl)pyridin-3-yl)-2-ethyl-18-isopropyl-5,5-dimethyl-8,14,17-trioxo-2,4,5,6,9,10,11,12,15,16,17,18,21,22-tetradecahydro-8H,14H,20H-9,13-epimino-1,29-etheno-15,26-methano-24,28-(metheno)pyrrolo[3,4-q][1,13,26]trioxa[4,7]diazacyclononacosin-21-yl)piperazine-1-carboxylate (60 mg, 0.055 mmol) in DCM (0.6 mL) was added trifluoroacetic acid (0.2 mL) dropwise at 0° C. under an atmosphere of argon. The resulting mixture was stirred at 0° C. for 1 hour. The reaction mixture was basified to pH 8 by the addition of sat. aq. NaHCO₃, extracted with DCM (3×10 mL), washed with brine (3×20 mL), dried over anhydrous Na₂SO₄, filtered, and concentrated under reduced pressure to give (9S,15S,18S,21 S)-3-(5-(4-cyclopropylpiperazin-1-yl)-2-((S)-1-methoxyethyl)pyridin-3-yl)-2-ethyl-18-isopropyl-5,5-dimethyl-21-(piperazin-1-yl)-2,4,5,6,9,10,11,12,15,16,21,22-dodecahydro-8H,14H,20H-9,13-epimino-1,29-etheno-15,26-methano-24,28-(metheno)pyrrolo[3,4-q][1,13,26]trioxa[4,7]diazacyclononacosine-8,14,17(18H)-trione (65 mg, crude). This material was taken directly to the next reaction without further purification. LCMS (ESI) m/z: [M+H] calcd for C₅₆H₇₇N₉O₇: 988.6; found 988.6.

Step 10. To a stirred solution of (9S,15S,18S,21S)-3-(5-(4-cyclopropylpiperazin-1-yl)-2-((S)-1-methoxyethyl)pyridin-3-yl)-2-ethyl-18-isopropyl-5,5-dimethyl-21-(piperazin-1-yl)-2,4,5,6,9,10,11,12,15,16,21,22-dodecahydro-8H,14H,20H-9,13-epimino-1,29-etheno-15,26-methano-24,28-(metheno)pyrrolo[3,4-q][1,13,26]trioxa[4,7]diazacyclononacosine-8,14,17(18H)-trione (65 mg, crude) and formaldehyde (19.8 mg, 0.660 mmol) in methanol (0.7 mL) were added AcOH (11.9 mg, 0.198 mmol) and NaBH₃CN (12.4 mg, 0.197 mmol) in portions at 0° C. under an atmosphere of argon. The resulting mixture was stirred at 0° C. for 1 hour. The reaction mixture was then quenched with sat. aq. K₃PO₄ at 0° C., extracted with EtOAc (3×20 mL), washed with brine (3×30 mL), dried over anhydrous Na₂SO₄, filtered, concentrated under reduced pressure, and purified by reversed-phase prep-HPLC to afford (9S,15S,18S,21S)-3-(5-(4-cyclopropylpiperazin-1-yl)-2-((S)-1-methoxyethyl)pyridin-3-yl)-2-ethyl-18-isopropyl-5,5-dimethyl-21-(4-methylpiperazin-1-yl)-2,4,5,6,9,10,11,12,15,16,21,22-dodecahydro-8H,14H,20H-9,13-epimino-1,29-etheno-15,26-methano-24,28-(metheno)pyrrolo[3,4-q][1,13,26]trioxa[4,7]diazacyclononacosine-8,14,17(18H)-trione (15.1 mg, 27% yield over 2 steps) as a white solid. LCMS (ESI) m/z: [M+H] calcd for C₅₇H₇₉N₉O₇: 1002.6; found 1002.7; ¹H NMR (400 MHz, Acetonitrile-d₃) δ 8.41 (d, J=5.2 Hz, 1H), 7.70 (d, J=7.9 Hz, 1H), 7.60-7.43 (m, 1H), 7.40-7.36 (m, 1H), 7.20 (d, J=3.4 Hz, 1H), 7.12-6.96 (m, 2H), 6.82-6.63 (m, 2H), 5.58 (d, J=10.3 Hz, 1H), 5.08 (s, 1H), 4.58 (d, J=13.4 Hz, 1H), 4.49-4.30 (m, 1H), 4.29-3.95 (m, 4H), 3.91-3.81 (m, 1H), 3.75 (t, J=9.7 Hz, 1H), 3.62 (d, J=10.5 Hz, 1H), 3.53-3.48 (m, 4H), 3.34 (t, J=9.4 Hz, OH), 3.20 (d, J=5.5 Hz, 5H), 3.13 (s, 2H), 3.10-2.97 (m, 1H), 2.88 (d, J=13.5 Hz, 2H), 2.77 (s, 3H), 2.72 (t, J=5.0 Hz, 5H), 2.66 (s, 2H), 2.33 (s, 3H), 2.19 (s, 1H), 2.01 (s, 1H), 1.77 (d, J=2.5 Hz, 1H), 1.66 (s, 1H), 1.40 (d, J=6.3 Hz, 2H), 1.36 (d, J=6.1 Hz, 3H), 1.30 (q, J=9.1, 8.1 Hz, 3H), 1.14 (s, 6H), 1.04 (d, J=6.8 Hz, 1H), 0.96 (t, J=7.5 Hz, 3H), 0.93-0.82 (m, 5H), 0.75 (d, J=6.5 Hz, 1H), 0.50 (d, J=9.8 Hz, 3H), 0.45 (d, J=5.8 Hz, 2H), 0.36 (s, 2H).

### Synthesis of Compound A17—(9S,15S,18S)-3-(5-(4-cyclopropylpiperazin-1-yl)-2-((S)-1-methoxyethyl)pyridin-3-yl)-2-ethyl-18-isopropyl-5,5-dimethyl-22-phenyl-2,4,5,6,9,10,11,12,15,16,20,21,22,23-tetradecahydro-8H,14H-9,13-epimino-1,29-etheno-15,26-methano-24,28-(metheno)pyrrolo[3,4-q][1,13]dioxa[4,7,27]triazacyclononacosine-8,14,17(18H)— trione

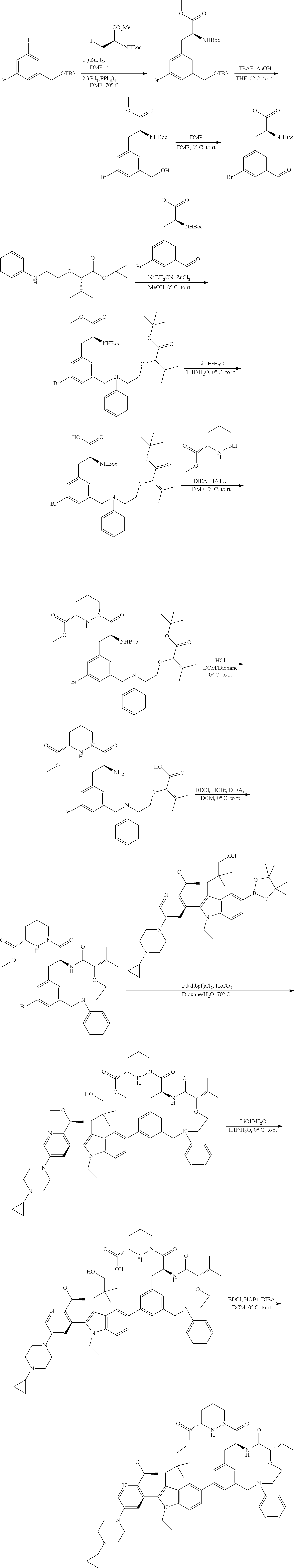

Step 1. To a stirred mixture of zinc (18.4 g, 281 mmol) and methyl (2R)-2-[(tert-butoxycarbonyl)amino]-3-iodopropanoate (15.4 g, 46.8 mmol) in DMF (60 mL) was added a solution of 12 (8.91 g, 35.1 mmol) in DMF (130 mL). The resulting mixture was stirred for 15 minutes at room temperature under an atmosphere of argon, after which time methyl (2R)-2-[(tert-butoxycarbonyl)amino]-3-iodopropanoate (30.8 g, 93.6 mmol) was added. The resulting mixture was stirred for 1 hour at room temperature. This mixture was filtered and the filtrate was added to a stirred mixture of ((3-bromo-5-iodobenzyl)oxy)(tert-butyl)dimethylsilane (10.0 g, 23.4 mmol) and Pd(PPh₃)₄ (1.60 g, 1.40 mmol) in DMF (18 mL). The resulting mixture was stirred for 2 hours at 70° C. under an atmosphere of argon. The reaction mixture was then quenched by the addition of ice water (100 mL) at 0° C., extracted with EtOAc (4×150 mL), treated with brine (3×50 mL), dried over anhydrous Na₂SO₄, concentrated under reduced pressure, and purified by reversed-phase flash column chromatography to give methyl (S)-3-(3-bromo-5-(((tert-butyldimethylsilyl)oxy)methyl)phenyl)-2-((tert-butoxycarbonyl)amino)propanoate (5.50 g, 47% yield) as a yellow oil. LCMS (ESI) m/z: [M+NH₄] calcd for C₂₂H₃₆BrNO₅Si: 521.2; found 521.0.

Step 2. To a stirred mixture of methyl (S)-3-(3-bromo-5-(((tert-butyldimethylsilyl)oxy)methyl)phenyl)-2-((tert-butoxycarbonyl)amino)propanoate (6.00 g, 11.9 mmol) in THF (60 mL) were added TBAF (6.24 g, 23.9 mmol) and AcOH (1.51 g, 25.1 mmol) at 0° C. The resulting mixture was stirred for 4 hours at room temperature. The reaction mixture was then quenched by the addition of ice water (100 mL) at 0° C., dried over anhydrous Na₂SO₄, filtered, concentrated under reduced pressure, and purified by normal phase flash column chromatography to give methyl (S)-3-(3-bromo-5-(hydroxymethyl)phenyl)-2-((tert-butoxycarbonyl)amino)propanoate (4.00 g, 86% yield) as a white solid. LCMS (ESI) m/z: [M+NH₄] calcd for C₁₆H₂₂BrNO₅: 407.1; found 406.9.

Step 3. To a stirred solution of methyl (S)-3-(3-bromo-5-(hydroxymethyl)phenyl)-2-((tert-butoxycarbonyl)amino)propanoate (4.30 g, 11.1 mmol) in DCM (43 mL) was added Dess-Martin periodinane (9.39 g, 22.2 mmol) at 0° C. The resulting mixture was stirred for 2 hours at room temperature. The reaction mixture was then quenched by the addition of sat. aq. Na₂S₂O₃ at 0° C., extracted with DCM (3×100 mL), washed with sat. aq. NaHCO₃, dried over anhydrous Na₂SO₄, filtered, concentrated under reduced pressure, and purified by normal phase flash column chromatography to afford methyl (S)-3-(3-bromo-5-formylphenyl)-2-((tert-butoxycarbonyl)amino)propanoate (4.00 g, 93% yield) as a white solid. LCMS (ESI) m/z: [M+NH₄] calcd for C₁₆H₂₀BrNO₅: 405.1; found 405.0.

Step 4. To a stirred mixture of methyl (S)-3-(3-bromo-5-formylphenyl)-2-((tert-butoxycarbonyl)amino)propanoate (3.00 g, 7.77 mmol) and tert-butyl (2S)-3-methyl-2-[2-(phenylamino)ethoxy]butanoate (910 mg, 3.11 mmol) in MeOH (30 mL) was added ZnCl₂ (422 mg, 6.32 mmol). The resulting mixture was stirred for 40 minutes at 0° C. NaBH₃CN (586 mg, 9.32 mmol) was then added at 0° C. and the resulting mixture was stirred for 2 hours at room temperature. The reaction mixture was then quenched by the addition of ice water (30 mL) at 0° C., extracted with EtOAc (3×50 mL), dried over anhydrous Na₂SO₄, filtered, concentrated under reduced pressure, and purified by normal phase flash column chromatography to give methyl (S)-1-((S)-3-(3-bromo-5-(((2-(((S)-1-(tert-butoxy)-3-methyl-1-oxobutan-2-yl)oxy)ethyl)(phenyl)amino)methyl)phenyl)-2-((tert-butoxycarbonyl)amino)propanoyl)hexahydropyridazine-3-carboxylate (1.70 g, 84% yield) as a yellow oil. LCMS (ESI) m/z: [M+H] calcd for C₃₃H₄₇BrN₂O₇: 665.3; found 665.0.

Step 5. To a stirred mixture of tert-butyl (S)-2-(2-((3-bromo-5-((S)-2-((tert-butoxycarbonyl)amino)-3-methoxy-3-oxopropyl)benzyl)(phenyl)amino)ethoxy)-3-methylbutanoate (2.95 g, 4.44 mmol) in THF (23 mL) and H₂O (7 mL) was added LiOH·H₂O (560 mg, 13.3 mmol) at 0° C. The resulting mixture was stirred for 2 hours at room temperature. The reaction mixture was quenched by the addition of ice water (20 mL) at 0° C., acidified to pH 6 with 1 M aq. HCl, extracted with EtOAc (3×50 mL), dried over anhydrous Na₂SO₄, filtered, and concentrated under reduced pressure to afford (2S)-3-(3-bromo-5-{[(2-{[(2S)-1-(tert-butoxy)-3-methyl-1-oxobutan-2-yl]oxy}ethyl)(phenyl)amino]methyl}phenyl)-2-[(tert-butoxycarbonyl)amino]propanoic acid (2.8 g, crude) as a white solid. This material was taken directly to the next reaction without further purification. LCMS (ESI) m/z: [M+H] calcd for C₃₂H₄₅BrN₂O₇: 649.2; found 649.5.

Step 6. To a stirred mixture of (2S)-3-(3-bromo-5-{[(2-{[(2S)-1-(tert-butoxy)-3-methyl-1-oxobutan-2-yl]oxy}ethyl)(phenyl)amino]methyl}phenyl)-2-[(tert-butoxycarbonyl)amino]propanoic acid (3.0 g, crude), methyl (3S)-1,2-diazinane-3-carboxylate (666 mg, 4.62 mmol, TFA salt), and DIEA (2.98 g, 23.1 mmol) in DMF (30 mL) was added HATU (2.11 g, 5.54 mmol) at 0° C. The resulting mixture was stirred for 1 hour at room temperature. The reaction mixture was then quenched by the addition of ice water (30 mL) at 0° C., extracted with EtOAc (3×100 mL), treated with brine (3×30 mL), dried over anhydrous Na₂SO₄, filtered, concentrated under reduced pressure, and purified by normal phase flash column chromatography to afford methyl (3S)-1-[(2S)-3-(3-bromo-5-{[(2-{[(2S)-1-(tert-butoxy)-3-methyl-1-oxobutan-2-yl]oxy}ethyl)(phenyl)amino]methyl}phenyl)-2-[(tert-butoxycarbonyl)amino]propanoyl]-1,2-diazinane-3-carboxylate (2.20 g, 60% yield over 2 steps) as a yellow solid. LCMS (ESI) m/z: [M+H] calcd for C₃₈H₅₅BrN₄O₈: 775.3; found 775.4.

Step 7. To a stirred solution of ethyl (3S)-1-[(2S)-3-(3-bromo-5-{[(2-{[(2S)-1-(tert-butoxy)-3-methyl-1-oxobutan-2-yl]oxy}ethyl)(phenyl)amino]methyl}phenyl)-2-[(tert-butoxycarbonyl)amino]propanoyl]-1,2-diazinane-3-carboxylate (2.50 g, 3.22 mmol) in DCM (25 mL) was added a solution of HCl in 1,4-dioxane (4.0 M, 25 mL, 25 mmol) at 0° C. The resulting mixture was stirred for 7 hours at room temperature and was then concentrated under reduced pressure to afford (S)-2-(2-((3-((S)-2-amino-3-((S)-3-(methoxycarbonyl)tetrahydropyridazin-1(2H)-yl)-3-oxopropyl)-5-bromobenzyl)(phenyl)amino)ethoxy)-3-methylbutanoic acid hydrochloride (3.0 g, crude) as a yellow solid. This material was taken directly to the next reaction without further purification. LCMS (ESI) m/z: [M+H] calcd for C₂₉H₃₉BrN₄O₆: 619.2; found 619.1.

Step 8. To a stirred mixture of (S)-2-(2-((3-((S)-2-amino-3-((S)-3-(methoxycarbonyl)tetrahydropyridazin-1(2H)-yl)-3-oxopropyl)-5-bromobenzyl)(phenyl)amino)ethoxy)-3-methylbutanoic acid hydrochloride (3.0 g, crude), EDCI (2.79 g, 18.0 mmol) and HOBt (3.27 g, 24.2 mmol) in DCM (30 mL) was added DIEA (2.50 g, 19.4 mmol) at 0° C. The resulting mixture was stirred for 16 hours at room temperature. The reaction mixture was quenched by the addition of ice water (30 mL) at 0° C., extracted with EtOAc (3×100 mL), washed with sat. aq. NH₄Cl (3×30 mL), dried over anhydrous Na₂SO₄, filtered, concentrated under reduced pressure, and purified by normal phase flash column chromatography to afford methyl (S)-1-((7S,10S)-15-bromo-7-isopropyl-8-oxo-3-phenyl-6-oxa-3,9-diaza-1(1,3)-benzenacycloundecaphane-10-carbonyl)hexahydropyridazine-3-carboxylate (1.00 g, 34% yield over 2 steps) as a yellow solid. LCMS (ESI) m/z: [M+H] calcd for C₂₉H₃₇BrN₄O₅: 600.2; found 600.9.

Step 9. A mixture of 3-[(2M)-2-[5-(4-cyclopropylpiperazin-1-yl)-2-[(1 S)-1-methoxyethyl]pyridin-3-yl]-1-ethyl-5-(4,4,5,5-tetramethyl-1,3,2-dioxaborolan-2-yl)indol-3-yl]-2,2-dimethylpropan-1-ol (493 mg, 0.799 mmol), methyl (S)-1-((7S,10S)-15-bromo-7-isopropyl-8-oxo-3-phenyl-6-oxa-3,9-diaza-1(1,3)-benzenacycloundecaphane-10-carbonyl)hexahydropyridazine-3-carboxylate (401 mg, 0.666 mmol), K₂CO₃ (230 mg, 1.67 mmol) and Pd(dppf)Cl₂ (48.8 mg, 0.067 mmol) in 1,4-dioxane (5 mL) and H₂O (1 mL) was stirred for 3 hours at 70° C. under at atmosphere of argon. The reaction mixture was then quenched by the addition of ice water (5 mL) at 0° C., extracted with EtOAc (3×50 mL), dried over anhydrous Na₂SO₄, filtered, concentrated under reduced pressure, and purified by normal phase flash column chromatography to afford methyl (S)-1-((7S,10S)-15-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-7-isopropyl-8-oxo-3-phenyl-6-oxa-3,9-diaza-1(1,3)-benzenacycloundecaphane-10-carbonyl)hexahydropyridazine-3-carboxylate (400 mg, 59% yield) as a white solid. LCMS (ESI) m/z: [M+H] calcd for C₅₉H₇₈N₈O₇: 1011.6; found 1011.6.

Step 10. To a stirred mixture of methyl (S)-1-((7S,10S)-15-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-7-isopropyl-8-oxo-3-phenyl-6-oxa-3,9-diaza-1(1,3)-benzenacycloundecaphane-10-carbonyl)hexahydropyridazine-3-carboxylate (400 mg, 0.40 mmol) in THF (4 mL) and H₂O (1 mL) was added LiOH·H₂O (49.8 mg, 1.19 mmol) at 0° C. The resulting mixture was stirred for 2 hours at room temperature. The reaction mixture was quenched by the addition (4 mL) at 0° C., acidified to pH 6 with 1 M aq. HCl, extracted with EtOAc (3×20 mL), dried over anhydrous Na₂SO₄, filtered, and concentrated under reduced pressure to afford (S)-1-((7S,10S)-15-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-7-isopropyl-8-oxo-3-phenyl-6-oxa-3,9-diaza-1(1,3)-benzenacycloundecaphane-10-carbonyl)hexahydropyridazine-3-carboxylic acid (380 mg, crude) as a white solid. This material was taken directly to the next reaction without further purification. LCMS (ESI) m/z: [M+H] calcd for C₅₈H₇₆N₈O₇: 997.6; found 997.6.

Step 11. To a stirred mixture of (S)-1-((7S,10S)-15-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-7-isopropyl-8-oxo-3-phenyl-6-oxa-3,9-diaza-1 (1,3)-benzenacycloundecaphane-10-carbonyl)hexahydropyridazine-3-carboxylic acid (400 mg, crude), EDCI (1.49 mg, 1.49 mmol) and HOBt (271 mg, 2.01 mmol) in DCM (40 mL) was added DIEA (2.07 g, 16.0 mmol) at 0° C. The resulting mixture was stirred for 16 hours at room temperature. The reaction mixture was quenched by the addition of ice water (40 mL) at 0° C., extracted with EtOAc (4×50 mL), washed with sat. aq. NH₄Cl (3×30 mL), dried over anhydrous Na₂SO₄, filtered, concentrated under reduced pressure, and purified by reversed phased prep-HPLC to afford (9S,15S,18S)-3-(5-(4-cyclopropylpiperazin-1-yl)-2-((S)-1-methoxyethyl)pyridin-3-yl)-2-ethyl-18-isopropyl-5,5-dimethyl-22-phenyl-2,4,5,6,9,10,11,12,15,16,20,21,22,23-tetradecahydro-8H,14H-9,13-epimino-1,29-etheno-15,26-methano-24,28-(metheno)pyrrolo[3,4-q][1,13]dioxa[4,7,27]triazacyclononacosine-8,14,17(18H)-trione (92.0 mg, 23% over 2 steps) as a white solid. LCMS (ESI) m/z: [M+H] calcd for C₅₈H₇₄N₈O₆: 979.6; found 979.6; ¹H NMR (400 MHz, DMSO-d₆) δ 8.51-8.42 (m, 2H), 8.11-8.05 (m, 1H), 7.78 (d, J=8.5 Hz, 1H), 7.67 (s, 2H), 7.60 (d, J=8.7 Hz, 1H), 7.28-7.19 (m, 2H), 7.14-7.09 (m, 2H), 6.76 (d, J=8.3 Hz, 2H), 6.59 (t, J=7.2 Hz, 1H), 5.48 (t, J=10.0 Hz, 1H), 5.34 (d, J=12.3 Hz, 1H), 4.62-4.44 (m, 2H), 4.26 (t, J=9.7 Hz, 2H), 4.12 (dd, J=13.8, 7.4 Hz, 3H), 3.89-3.73 (m, 2H), 3.66 (d, J=10.9 Hz, 1H), 3.62-3.44 (m, 3H), 3.28-3.14 (m, 5H), 3.11 (s, 3H), 2.92-2.73 (m, 3H), 2.73-2.56 (m, 6H), 2.06 (d, J=14.3 Hz, 1H), 1.94 (dt, J=13.6, 7.3 Hz, 1H), 1.88-1.62 (m, 3H), 1.61-1.48 (m, 1H), 1.35 (d, J=6.1 Hz, 3H), 1.25 (d, J=14.9 Hz, 1H), 1.05-0.89 (m, 6H), 0.81 (s, 4H), 0.70 (d, J=6.6 Hz, 3H), 0.50 (s, 3H), 0.44 (dd, J=6.5, 2.2 Hz, 2H), 0.34 (q, J=3.2, 2.8 Hz, 2H).

### Synthesis of Compound A1—(9S,15S,18R,21R)-3-(5-(4-cyclopropylpiperazin-1-yl)-2-((S)-1-methoxyethyl)pyridin-3-yl)-2-ethyl-18-isopropyl-5,5-dimethyl-21-(pyridin-4-yl)-2,4,5,6,9,10,11,12,15,16,18,19,22,23-tetradecahydro-8H,21H-9,13-epimino-1,29-etheno-15,26-methano-24,28-(metheno)pyrrolo[3,4-1][1,17]dioxa[4,23,26]triazacyclononacosine-8,14,17-trione

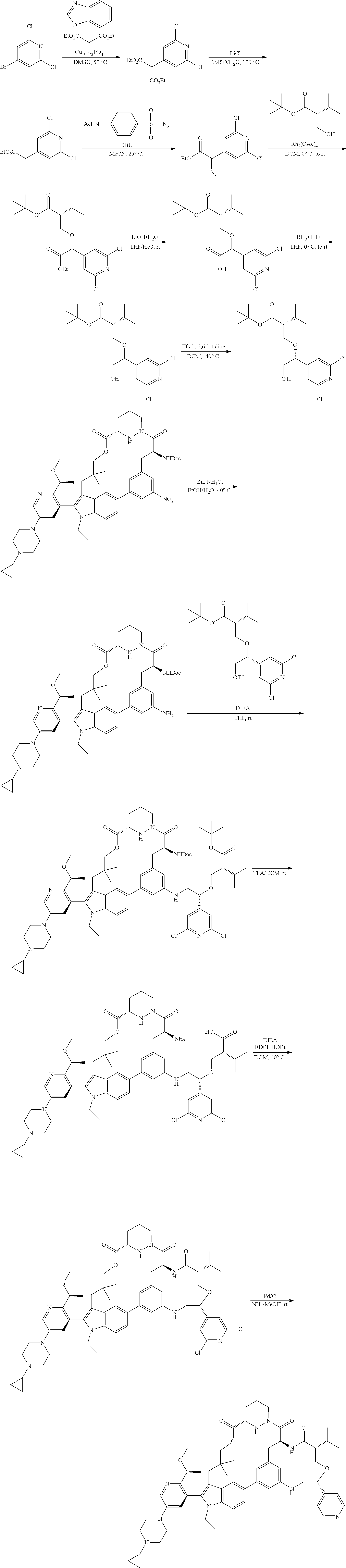

Step 1. To a mixture of 4-bromo-2,6-dichloropyridine (10.0 g, 44.1 mmol), CuI (839 mg, 4.41 mmol), K₃PO₄ (23.4 g, 110 mmol), and 1,3-benzoxazole (1.05 g, 8.82 mmol) in DMSO (100 mL) was added diethyl malonate (9.97 mL, 65.7 mmol) at room temperature. The resulting mixture was stirred for 3 hours at 50° C. under an atmosphere of argon. The reaction was quenched by the addition of H₂O (200 mL) and the resulting mixture was extracted with EtOAc (3×100 mL), treated with brine (100 mL), dried over anhydrous Na₂SO₄, filtered, concentrated under reduced pressured, and purified by normal phase flash column chromatography to afford diethyl 2-(2,6-dichloropyridin-4-yl)malonate (8.29 g, 61% yield) as a yellow oil. LCMS (ESI) m/z: [M+H] calcd for C₁₂H₁₃Cl₂NO₄: 306.0; found 306.0.

Step 2. A mixture of diethyl 2-(2,6-dichloropyridin-4-yl)malonate (8.29 g, 27.1 mmol) and LiCl (2.87 g, 67.7 mmol) in H₂O (50 mL) in DMSO (50 mL) was stirred for 16 hours at 120° C. The reaction was quenched by the addition of H₂O (100 mL) and the resulting mixture was extracted with EtOAc (3×100 mL), treated with brine (100 mL), dried over anhydrous Na₂SO₄, filtered, concentrated under reduced pressure and purified by normal phase column chromatography to give ethyl 2-(2,6-dichloropyridin-4-yl)acetate (3.77 g, 59% yield) as a yellow oil. LCMS (ESI) m/z: [M+H] calcd for C₉H₉Cl₂NO₂: 234.0; found 234.0.

Step 3. To a stirred solution of ethyl 2-(2,6-dichloropyridin-4-yl)acetate (3.70 g, 15.8 mmol) and 4-acetamidobenzenesulfonyl azide (3.80 g, 15.8 mmol) in MeCN (100 mL) was added DBU (7.08 mL, 47.4 mmol). The resulting mixture was stirred for 1 hour at room temperature. The reaction mixture was quenched by the addition of H₂O (50 mL) and the resulting mixture was extracted with EtOAc (3×50 mL), dried over anhydrous Na₂SO₄, filtered, concentrated under reduced pressure, and purified by normal phase flash column chromatography to give ethyl 2-diazo-2-(2,6-dichloropyridin-4-yl)acetate (1.30 g, 32% yield) as a yellow solid. LCMS (ESI) m/z: [M+H] calcd for C₉H₇Cl₂N₃O₂: 260.0; found 260.0.

Step 4. To a stirred solution of ethyl 2-diazo-2-(2,6-dichloropyridin-4-yl)acetate (1.30 g, 5.00 mmol) and Rh₂(OAc)₄ (44.2 mg, 0.100 mmol) in DCM (10 mL) was added a solution tert-butyl (R)-2-(hydroxymethyl)-3-methylbutanoate (1.41 g, 7.50 mmol) in DCM (10 mL) dropwise at 0° C. The resulting mixture was stirred for 1 hour at room temperature under an atmosphere of N₂. The resulting mixture was concentrated under reduced pressure and purified by normal phase flash column chromatography to give tert-butyl (2R)-2-((1-(2,6-dichloropyridin-4-yl)-2-ethoxy-2-oxoethoxy)methyl)-3-methylbutanoate (1.67 g, 79% yield) as a colorless oil. LCMS (ESI) m/z: [M+H] calcd for C₁₉H₂₇Cl₂NO₅: 420.1; found 420.1.

Step 5. A mixture of tert-butyl (2R)-2-((1-(2,6-dichloropyridin-4-yl)-2-ethoxy-2-oxoethoxy)methyl)-3-methylbutanoate (1.40 g, 3.33 mmol) and LiOH·H₂O (559 mg, 13.3 mmol) in THF (10 mL) and H₂O (10 mL) was stirred for 30 minutes at room temperature. The resulting mixture was acidified to pH 5 by the addition of citric acid and was then extracted with DCM (3×50 mL), treated with brine (30 mL), dried over anhydrous Na₂SO₄, filtered, and concentrated under reduced pressure to give 2-((R)-2-(tert-butoxycarbonyl)-3-methylbutoxy)-2-(2,6-dichloropyridin-4-yl)acetic acid (1.20 g, crude) as a colorless oil. This material was taken directly to the next reaction without further purification.

Step 6. To a stirred solution of 2-((R)-2-(tert-butoxycarbonyl)-3-methylbutoxy)-2-(2,6-dichloropyridin-4-yl)acetic acid (1.20 g, crude) in THF (15 mL) was added BH₃·THF (12.2 mL, 12.2 mmol) dropwise at 0° C. The reaction mixture was stirred for 16 hours at room temperature under an atmosphere of N₂. The mixture was then quenched by the addition of sat. aq. NaHCO₃ (30 mL), extracted with EtOAc (2×30 mL), treated with brine (20 mL), dried over anhydrous Na₂SO₄, filtered, concentrated under reduced pressure, and purified by reversed-phase flash column chromatography to give tert-butyl (2R)-2-((1-(2,6-dichloropyridin-4-yl)-2-hydroxyethoxy)methyl)-3-methylbutanoate as a mixture of epimers. This material was then subjected to chiral prep-SFC purification to yield tert-butyl (R)-2-(((S)-1-(2,6-dichloropyridin-4-yl)-2-hydroxyethoxy)methyl)-3-methylbutanoate (420 mg, 33% yield over 2 steps, assumed absolute configuration) as a colorless oil and tert-butyl (R)-2-(((R)-1-(2,6-dichloropyridin-4-yl)-2-hydroxyethoxy)methyl)-3-methylbutanoate (240 mg, 19% yield over 2 steps, assumed absolute configuration) as a colorless oil. LCMS (ESI) m/z: [M+H] calcd for C₁₇H₂₅Cl₂NO₄: 378.1; found 378.3.

Step 7. To a stirred solution of tert-butyl (R)-2-(((R)-1-(2,6-dichloropyridin-4-yl)-2-hydroxyethoxy)methyl)-3-methylbutanoate (200 mg, 0.529 mmol) and 2,6-lutidine (246 μL, 2.12 mmol) in DCM (5 mL) was added Tf₂O (2.00 mL, 0.800 mmol) dropwise at −40° C. under an atmosphere of argon. The resulting mixture was stirred for 10 minutes at −40° C. under an atmosphere of argon. The reaction mixture was then quenched by the addition of sat. aq. NH₄Cl (10 mL) at 0° C., extracted with DCM (2×10 mL), treated with brine (10 mL), dried over anhydrous Na₂SO₄, filtered, and concentrated under reduced pressure to give tert-butyl (R)-2-(((R)-1-(2,6-dichloropyridin-4-yl)-2-(((trifluoromethyl)sulfonyl)oxy)ethoxy)methyl)-3-methylbutanoate (650 mg, crude) as a yellow oil. This material was taken directly to the next reaction without further purification. LCMS (ESI) m/z: [M+H] calcd for C₁₈H₂₄Cl₂F₃NO₆S: 510.1; found 510.1.

Step 8. To a stirred mixture of tert-butyl ((6³S,4S)-1²-(5-(4-cyclopropylpiperazin-1-yl)-2-((S)-1-methoxyethyl)pyridin-3-yl)-1¹-ethyl-10,10-dimethyl-2⁵-nitro-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (500 mg, 0.560 mmol) and NH₄Cl (240 mg, 4.48 mmol) in EtOH (15 mL) and H₂O (5 mL) was added zinc powder (146 mg, 2.24 mmol) at room temperature. The reaction mixture was stirred for 16 h at 40° C. under an atmosphere of argon. The resulting mixture was then filtered, concentrated under reduced pressure, and purified by reversed-phase flash chromatography to give tert-butyl ((6³S,4S)-2⁵-amino-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (390 mg, 81% yield) as a yellow solid. LCMS (ESI) m/z: [M+H] calcd for C₄₉H₆₆N₈O₆: 863.5; found 863.5.

Step 9. A solution of DIEA (0.61 mL, 3.480 mmol), tert-butyl ((6³S,4S)-2⁵-amino-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (300 mg, 0.348 mmol) and tert-butyl (R)-2-(((R)-1-(2,6-dichloropyridin-4-yl)-2-(((trifluoromethyl)sulfonyl)oxy)ethoxy)methyl)-3-methylbutanoate (600 mg, crude) in THF (5 mL) was stirred for 16 hours at room temperature. The resulting mixture was concentrated under reduced pressure and was then purified by reversed-phase flash chromatography to yield tert-butyl (2R)-2-(((1R)-2-(((6³S,4S)-4-((tert-butoxycarbonyl)amino)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)amino)-1-(2,6-dichloropyridin-4-yl)ethoxy)methyl)-3-methylbutanoate (350 mg, 82% yield) as a yellow solid. LCMS (ESI) m/z: [M+H] calcd for C₆₆H₈₉Cl₂N₉O₉: 1222.6; found 1222.5.

Step 10. To a stirred solution of tert-butyl (2R)-2-(((1R)-2-(((6³S,4S)-4-((tert-butoxycarbonyl)amino)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-25-yl)amino)-1-(2,6-dichloropyridin-4-yl)ethoxy)methyl)-3-methylbutanoate (310 mg, 0.253 mmol) in DCM (2 mL) was added TFA (2 mL) dropwise. The reaction mixture was stirred for 1 hour at room temperature. The resulting mixture was concentrated under reduced pressure, neutralized to pH 7 by the addition of sat. aq. NaHCO₃, extracted with DCM (3×10 mL), treated with brine (10 mL), dried over anhydrous Na₂SO₄, filtered, and concentrated under reduced pressure to give (2R)-2-(((1R)-2-(((6³S,4S)-4-amino-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)amino)-1-(2,6-dichloropyridin-4-yl)ethoxy)methyl)-3-methylbutanoic acid (230 mg, crude) as a yellow solid. This material was taken directly to the next reaction without further purification.

Step 11. To a stirred solution of DIEA (1.30 mL, 7.45 mmol), EDCI (952 mg, 4.96 mmol) and HOBt (336 mg, 2.48 mmol) in DCM (150 mL) was added dropwise a solution of (2R)-2-(((1R)-2-(((6³S,4S)-4-amino-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)amino)-1-(2,6-dichloropyridin-4-yl)ethoxy)methyl)-3-methylbutanoic acid (265 mg, crude) in DCM (150 mL) dropwise at 40° C. The reaction mixture was stirred for 1 hour at 40° C. The resulting mixture was concentrated to around 30 mL under reduced pressure and was then treated with brine (2×100 mL), dried over anhydrous Na₂SO₄, filtered, concentrated under reduced pressure, and purified by reversed-phase flash chromatography to yield (9S,15S,18R,21R)-3-(5-(4-cyclopropylpiperazin-1-yl)-2-((S)-1-methoxyethyl)pyridin-3-yl)-21-(2,6-dichloropyridin-4-yl)-2-ethyl-18-isopropyl-5,5-dimethyl-2,4,5,6,9,10,11,12,15,16,18,19,22,23-tetradecahydro-8H,21H-9,13-epimino-1,29-etheno-15,26-methano-24,28-(metheno)pyrrolo[3,4-l][1,17]dioxa[4,23,26]triazacyclononacosine-8,14,17-trione (200 mg, 66% yield over 2 steps) as a yellow solid. LCMS (ESI) m/z: [M+H] calcd for C₅₇H₇₁C₁₂N₉O₆: 1048.5; found 1048.5.

Step 12. A mixture of (9S,15S,18R,21R)-3-(5-(4-cyclopropylpiperazin-1-yl)-2-((S)-1-methoxyethyl)pyridin-3-yl)-21-(2,6-dichloropyridin-4-yl)-2-ethyl-18-isopropyl-5,5-dimethyl-2,4,5,6,9,10,11,12,15,16,18,19,22,23-tetradecahydro-8H,21H-9,13-epimino-1,29-etheno-15,26-methano-24,28-(metheno)pyrrolo[3,4-l][1,17]dioxa[4,23,26]triazacyclononacosine-8,14,17-trione (190 mg, 0.181 mmol) and 10% Pd/C (95 mg) was stirred in a solution of NH₃ in MeOH (7 M, 10 mL) for 1 hour at room temperature under an atmosphere of H₂ gas. The resulting mixture was filtered, and the filter cake was washed with MeOH (2×5 mL). The filtrate was concentrated under reduced pressure and purified by reversed-phase flash column chromatography to give (9S,15S,18R,21R)-3-(5-(4-cyclopropylpiperazin-1-yl)-2-((S)-1-methoxyethyl)pyridin-3-yl)-2-ethyl-18-isopropyl-5,5-dimethyl-21-(pyridin-4-yl)-2,4,5,6,9,10,11,12,15,16,18,19,22,23-tetradecahydro-8H,21H-9,13-epimino-1,29-etheno-15,26-methano-24,28-(metheno)pyrrolo[3,4-l][1,17]dioxa[4,23,26]triazacyclononacosine-8,14,17-trione (41.4 mg, 22% yield) as a white solid. LCMS (ESI) m/z: [M+H] calcd for C₅₇H₇₃N₉O₆: 980.6; found 980.6; ¹H NMR (400 MHz, DMSO-d₆) δ 8.42-8.34 (m, 2H), 8.25-8.16 (m, 2H), 7.83 (s, 1H), 7.40-7.31 (m, 2H), 7.18 (d, J=6.1 Hz, 2H), 6.96 (d, J=2.9 Hz, 1H), 6.83 (s, 1H), 6.74 (d, J=3.5 Hz, 2H), 5.77 (t, J=6.4 Hz, 1H), 5.40 (t, J=9.8 Hz, 1H), 4.95 (d, J=12.3 Hz, 1H), 4.19-4.13 (m, 1H), 4.12-4.00 (m, 2H), 3.99-3.88 (m, 3H), 3.57 (d, J=10.8 Hz, 1H), 3.39 (d, J=10.8 Hz, 1H), 3.16-3.03 (m, 4H), 3.03-2.94 (m, 7H), 2.90-2.78 (m, 2H), 2.62-2.50 (m, 2H), 2.50-2.38 (m, 5H), 2.33-2.15 (m, 2H), 1.92-1.82 (m, 1H), 1.66-1.48 (m, 2H), 1.47-1.39 (m, 1H), 1.38-1.24 (m, 2H), 1.12 (d, J=6.1 Hz, 3H), 0.71-0.62 (m, 6H), 0.52 (t, J=7.6 Hz, 6H), 0.25-0.08 (m, 6H).

### Synthesis of Compound A13—(9S,15S,18S,20S)-2-ethyl-18-isopropyl-3-(2-((S)-1-methoxyethyl)-5-(4-methylpiperazin-1-yl)pyridin-3-yl)-5,5,19-trimethyl-20-phenyl-2,4,5,6,9,10,11,12,15,16,18,19,20,21,22,23-hexadecahydro-8H-9,13-epimino-1,29-etheno-15,26-methano-24,28-(metheno)pyrrolo[3,4-y][1]oxa[7,10,13,17]tetraazacyclononacosine-8,14,17-trione

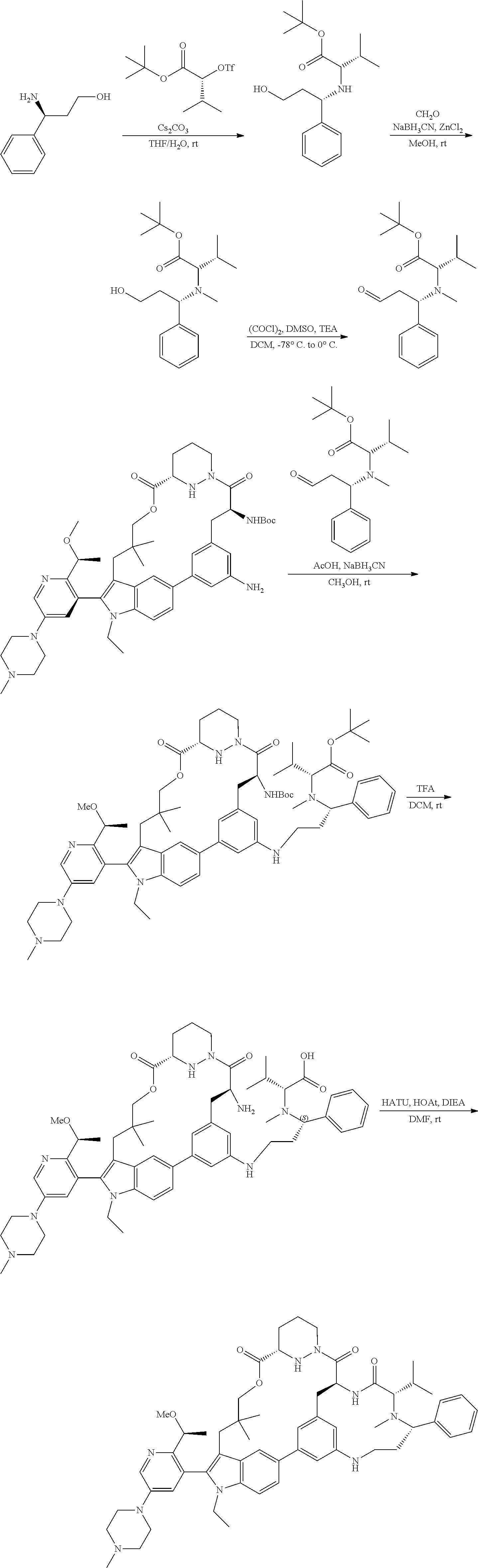

Step 1. To a stirred solution of (S)-3-amino-3-phenylpropan-1-ol (2.50 g, 16.5 mmol) and tert-butyl (R)-3-methyl-2-(((trifluoromethyl)sulfonyl)oxy)butanoate (9.12 g, 29.8 mmol) in THF (25 mL) and H₂O (25 mL) was added Cs₂CO₃ (8.08 g, 24.8 mmol) in portions at room temperature. The resulting mixture was stirred for 18 hours at room temperature. The reaction mixture was then extracted with EtOAc (3×50 mL), dried over anhydrous Na₂SO₄, filtered, concentrated under reduced pressure, and purified by normal phase flash column chromatography to afford tert-butyl ((S)-3-hydroxy-1-phenylpropyl)-L-valinate (3.34 g, 59% yield) as a clear oil. LCMS (ESI) m/z: [M+H] calcd for C₁₈H₂₉NO₃: 308.2; found 308.1.

Step 2. To a stirred solution of tert-butyl ((S)-3-hydroxy-1-phenylpropyl)-L-valinate (3.34 g, 10.9 mmol) and formaldehyde (9.21 g, 107 mmol, 35% aq. solution) in CH₃OH (34 mL) were added ZnCl₂ (14.8 g, 109 mmol) and NaBH₃CN (6.83 g, 109 mmol) in portions at 0° C. under an atmosphere of N₂. The resulting mixture was stirred for 2 hours at room temperature. The reaction mixture was then quenched by the addition of sat. aq. NaHCO₃ at 0° C., extracted with a 10:1 vol. mixture of DCM:CH₃OH (3×90 mL), dried over anhydrous Na₂SO₄, filtered, concentrated under reduced pressure, and purified by normal phase flash column chromatography to afford tert-butyl N—((S)-3-hydroxy-1-phenylpropyl)-N-methyl-L-valinate (3.23 g, 83% yield) as a colorless oil. LCMS (ESI) m/z: [M+H] calcd for C₁₉H₃₁NO₃: 322.2; found 322.1.

Step 3. To a stirred solution of oxalyl chloride (355 mg, 2.80 mmol) in DCM (18 mL) was added DMSO (437 mg, 5.60 mmol) dropwise over 30 min at −78° C. under an atmosphere of N₂. Subsequently, tert-butyl N—((S)-3-hydroxy-1-phenylpropyl)-N-methyl-L-valinate (600 mg, 1.87 mmol) was added in portions at −78° C. The resulting mixture was stirred for 1 hour at −78° C., after which time triethylamine (1.50 g, 14.9 mmol) was added in portions at −78° C. The resulting mixture was stirred for 10 minutes at 0° C. under an atmosphere of N₂. The reaction mixture was then quenched by the addition of H₂O at −30° C. and the aqueous phase was extracted with DCM (3×20 mL), treated with brine (3×30 mL), and concentrated under reduced pressure to give tert-butyl N-methyl-N—((S)-3-oxo-1-phenylpropyl)-L-valinate (572 mg, crude). This material was taken directly to the next reaction without further purification. LCMS (ESI) m/z: [M+H] calcd for C₁₉H₂₉NO: 320.2; found 320.1.

Step 4. To a stirred solution of tert-butyl ((6³S,4S)-2⁵-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (500 mg, 0.597 mmol) and tert-butyl N-methyl-N—((S)-3-oxo-1-phenylpropyl)-L-valinate (572 mg, crude) in DCM (63 mL) and CH₃OH (63 mL) was added AcOH (359 mg, 5.97 mmol) dropwise over 10 minutes at 0° C. followed by NaBH₃CN (375 mg, 5.97 mmol) in portions at 0° C. under an atmosphere of N₂. The resulting mixture was stirred for 2 hours at 0° C. The reaction mixture was then quenched by the addition of sat. aq. NaHCO₃ (150 mL) at 0° C., extracted with a 10:1 vol. mixture of DCM/CH₃OH (3×150 mL), dried over anhydrous Na₂SO₄, filtered, concentrated under reduced pressure, and purified by reversed-phase flash column chromatography to afford tert-butyl N-((1 S)-3-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)amino)-1-phenylpropyl)-N-methyl-D-valinate (588 mg, 78% yield) as a light yellow solid. LCMS (ESI) m/z: [M+H] calcd for C₆₆H₉₃N₉O₈: 1140.7; found 1140.7.

Step 5. To a stirred solution of tert-butyl N-((1 S)-3-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)amino)-1-phenylpropyl)-N-methyl-D-valinate (588 mg, 0.516 mmol) in DCM (6 mL) was added TFA (6 mL) in portions at 0° C. The resulting mixture was stirred for 16 hours at room temperature and was then concentrated under reduced pressure to afford N-((1 S)-3-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-1-phenylpropyl)-N-methyl-D-valine (970 mg, crude) as a light yellow powder. This material was taken directly to the next reaction without further purification. LCMS (ESI) m/z: [M+H] calcd for C₅₇H₇₇N₉O₆: 984.6; found 984.5.

Step 6. To a stirred solution of N-((1S)-3-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-1-phenylpropyl)-N-methyl-D-valine (970 mg, crude) and DIEA (1.29 g, 10.0 mmol) in DMF (97 mL) were added HATU (380 mg, 1.00 mmol) and HOAt (136 mg, 1.00 mmol) in portions at 0° C. The resulting mixture was stirred for 2 hours at room temperature. The reaction mixture was quenched by the addition of H₂O at 0° C., extracted with EtOAc (3×100 mL), treated with brine (3×150 mL), dried over anhydrous Na₂SO₄, filtered, concentrated under reduced pressure, and purified by reversed-phase prep-HPLC to afford (9S,15S,18S,20S)-2-ethyl-18-isopropyl-3-(2-((S)-1-methoxyethyl)-5-(4-methylpiperazin-1-yl)pyridin-3-yl)-5,5,19-trimethyl-20-phenyl-2,4,5,6,9,10,11,12,15,16,18,19,20,21,22,23-hexadecahydro-8H-9,13-epimino-1,29-etheno-15,26-methano-24,28-(metheno)pyrrolo[3,4-y][1]oxa[7,10,13,17]tetraazacyclononacosine-8,14,17-trione (40.0 mg, 8% yield over 2 steps) as a white solid. LCMS (ESI) m/z: [M+H] calcd for C₅₇H₇₅N₉O₅: 966.6; found 966.6; ¹H NMR (400 MHz, DMSO-d₆) δ 8.56-8.33 (m, 2H), 8.07 (s, 1H), 7.57 (s, 2H), 7.39-7.33 (m, 4H), 7.29-7.14 (m, 2H), 6.96 (s, 2H), 6.59 (s, 1H), 5.81 (s, 1H), 5.41 (t, J=9.7 Hz, 1H), 5.18-4.95 (m, 1H), 4.29-4.22 (m, 2H), 4.21-4.01 (m, 4H), 3.95-3.75 (m, 2H), 3.61 (d, J=10.6 Hz, 1H), 3.27 (br s, 5H), 3.23 (s, 3H), 3.18-2.97 (m, 2H), 2.96-2.80 (m, 3H), 2.66-2.60 (m, 1H), 2.47 (s, 3H), 2.42-2.31 (m, 1H), 2.22 (br s, 4H), 2.11-2.01 (m, 4H), 1.78-1.70 (m, 3H), 1.59-1.51 (m 1H), 1.33 (d, J=6.1 Hz, 3H), 0.90 (t, J=6.7 Hz, 6H), 0.87-0.72 (m, 6H), 0.37 (s, 3H).

### Synthesis of Compound A33—(9S,15S,18R)-3-(5-(4-cyclopropylpiperazin-1-yl)-2-((S)-1-methoxyethyl)pyridin-3-yl)-2-ethyl-18-isopropyl-5,5,20-trimethyl-21-phenyl-2,4,5,6,9,10,11,12,15,16,19,20,21,22-tetradecahydro-8H,14H-9,13-epimino-1,29-etheno-15,26-methano-24,28-(metheno)pyrrolo[3,4-u][1,17]dioxa[4,8,11]triazacyclononacosine-8,14,17(18H)-trione

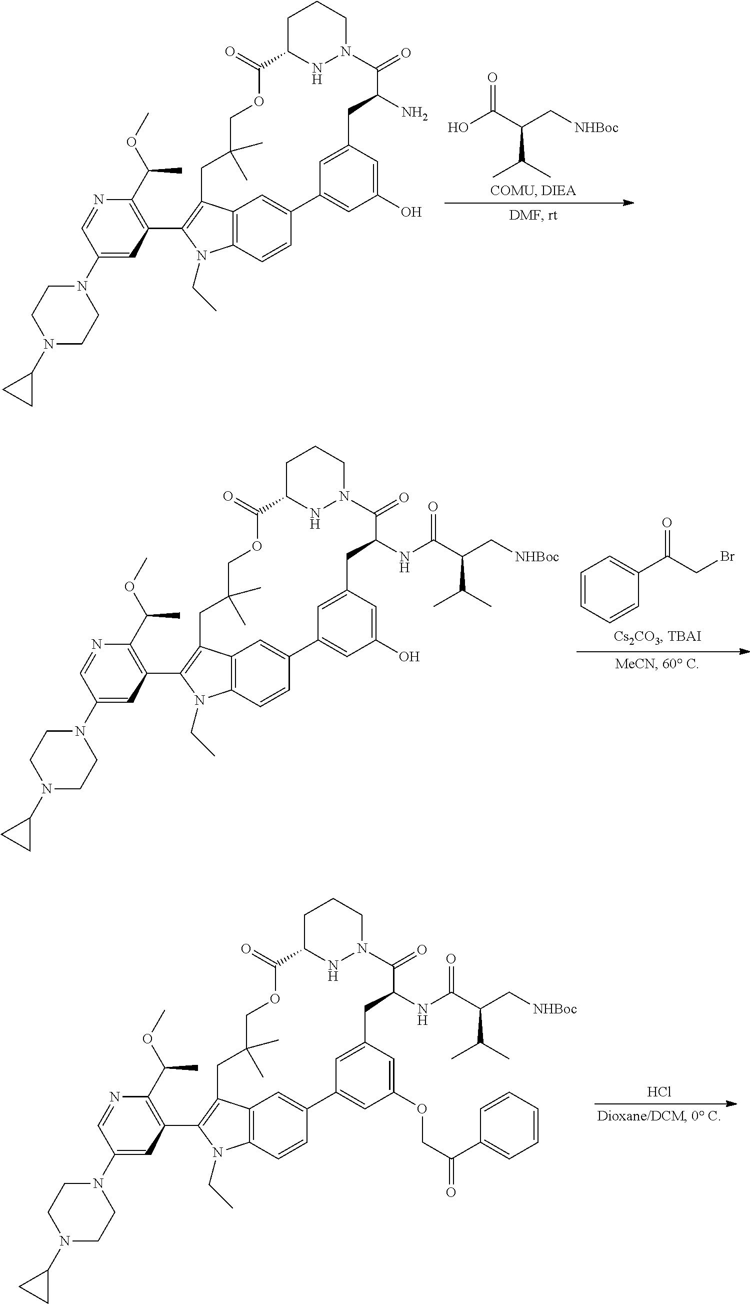

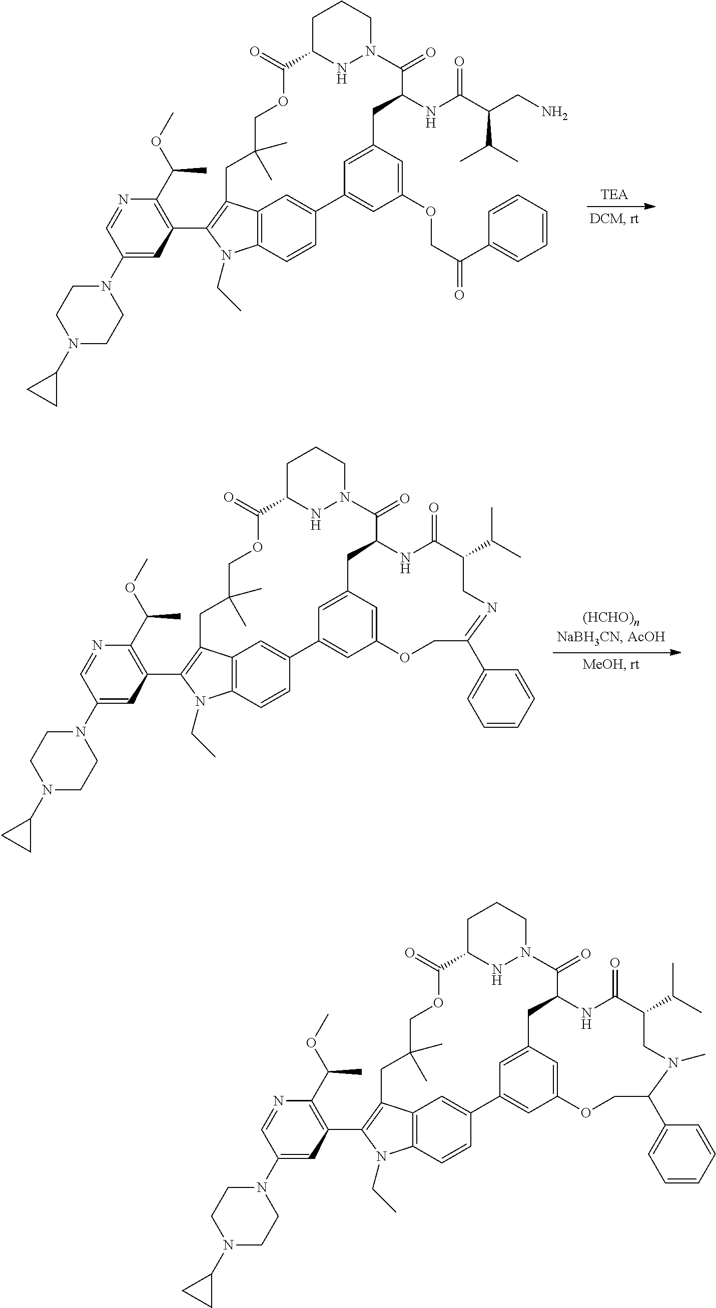

Step 1. To a stirred solution of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.00 g, 1.31 mmol) in DMF (10 mL) was added DIEA (1.69 g, 13.1 mmol) followed by (2R)-2-{[(tert-butoxycarbonyl)amino]methyl}-3-methylbutanoic acid (454 mg, 1.96 mmol) and COMU (673 mg, 1.57 mmol) dropwise at 0° C. under an atmosphere of N₂. The resulting mixture was stirred for 1 hour at room temperature. The reaction mixture was then extracted with EtOAc (3×5 mL), treated with brine (3×5 mL), dried over anhydrous Na₂SO₄, filtered, concentrated under reduced pressure, and purified by normal phase flash column chromatography to give tert-butyl ((2R)-2-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamoyl)-3-methylbutyl)carbamate (750 mg, 53% yield) as a yellow oil. LCMS (ESI) m/z: [M+H] calcd for C₅₅H₇₆N₈O₈: 977.6; found 978.1.

Step 2. To a stirred solution of tert-butyl ((2R)-2-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamoyl)-3-methylbutyl)carbamate (500 mg, 0.512 mmol) in MeCN (5 mL) were added Cs₂CO₃ (417 mg, 1.28 mmol) and TBAI (227 mg, 0.614 mmol), followed by 2-bromoacetophenone (122 mg, 0.614 mmol) in portions at 0° C. under an atmosphere of N₂. The resulting mixture was stirred for 2 hours at room temperature. The reaction mixture was extracted with EtOAc (3×20 mL), treated with brine (3×30 mL), dried over anhydrous Na₂SO₄, filtered, concentrated under reduced pressure, and purified by reversed-phase flash column chromatography to give tert-butyl ((2R)-2-(((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2⁵-(2-oxo-2-phenylethoxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamoyl)-3-methylbutyl)carbamate (110 mg, 18% yield) as a light yellow oil. LCMS (ESI) m/z: [M+H] calcd for C₆₃H₈₂N₈O₉: 1095.6; found 1095.6.

Step 3. tert-butyl ((2R)-2-(((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2⁵-(2-oxo-2-phenylethoxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamoyl)-3-methylbutyl)carbamate (90.0 mg, 0.082 mmol) was added to a solution of HCl in 1,4-dioxane (1 mL) and DCM (1 mL). The resulting mixture was stirred for 2 hours at 0° C. under an atmosphere of N₂, and was then concentrated under reduced pressure to give (2R)-2-(aminomethyl)-N-((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2⁵-(2-oxo-2-phenylethoxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (95.0 mg, crude) as a light yellow oil. This material was taken directly to the next reaction without further purification. LCMS (ESI) m/z: [M/2+H] calcd for C₅₈H₇₄N₈O₇: 498.3; found 498.6.

Step 4. To a stirred solution of (2R)-2-(aminomethyl)-N-((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2⁵-(2-oxo-2-phenylethoxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (100 mg, crude) in DCM (1 mL) was added trimethylamine (1.0 mL) dropwise over 10 minutes at 0° C. under an atmosphere of N₂. The resulting mixture was stirred for 2 hours at room temperature. The reaction mixture was then quenched by the addition of ice water at 0° C., extracted with EtOAc (3×10 mL), treated with brine (3×10 mL), dried over anhydrous Na₂SO₄, filtered, concentrated under reduced pressure, and purified by normal phase prep-TLC to afford (9S,15S,18R)-3-(5-(4-cyclopropylpiperazin-1-yl)-2-((S)-1-methoxyethyl)pyridin-3-yl)-2-ethyl-18-isopropyl-5,5-dimethyl-21-phenyl-2,4,5,6,9,10,11,12,15,16,19,22-dodecahydro-8H,14H-9,13-epimino-1,29-etheno-15,26-methano-24,28-(metheno)pyrrolo[3,4-u][1,17]dioxa[4,8,11]triazacyclononacosine-8,14,17(18H)-trione (55.0 mg, 62% yield over 2 steps) as a light yellow oil. LCMS (ESI) m/z: [M+H] calcd for C₅₈H₇₂N₈O₆: 977.6; found 977.9.

Step 5. To a stirred solution of (9S,15S,18R)-3-(5-(4-cyclopropylpiperazin-1-yl)-2-((S)-1-methoxyethyl)pyridin-3-yl)-2-ethyl-18-isopropyl-5,5-dimethyl-21-phenyl-2,4,5,6,9,10,11,12,15,16,19,22-dodecahydro-8H,14H-9,13-epimino-1,29-etheno-15,26-methano-24,28-(metheno)pyrrolo[3,4-u][1,17]dioxa[4,8,11]triazacyclononacosine-8,14,17(18H)-trione (50.0 mg, 0.051 mmol) in MeOH (0.5 mL) was added AcOH (6.13 mg, 0.102 mmol) followed by paraformaldehyde (16.1 mg, 0.153 mmol) and NaBH₃CN (6.42 mg, 0.102 mmol) in portions at 0° C. under an atmosphere of N₂. The resulting mixture was stirred for 2 hours at room temperature. The reaction mixture was then quenched by the addition of ice water, extracted with EtOAc (3×20 mL), treated with brine (3×20 mL), dried over anhydrous Na₂SO₄, filtered, concentrated under reduced pressure, and purified by reversed-phase prep-HPLC to afford (9S,15S,18R)-3-(5-(4-cyclopropylpiperazin-1-yl)-2-((S)-1-methoxyethyl)pyridin-3-yl)-2-ethyl-18-isopropyl-5,5,20-trimethyl-21-phenyl-2,4,5,6,9,10,11,12,15,16,19,20,21,22-tetradecahydro-8H,14H-9,13-epimino-1,29-etheno-15,26-methano-24,28-(metheno)pyrrolo[3,4-u][1,17]dioxa[4,8,11]triazacyclononacosine-8,14,17(18H)-trione (17.1 mg, 33% yield) as an off-white solid. LCMS (ESI) m/z: [M+H] calcd for C₅₉H₇₆N₈O₆: 993.6; found 993.6; ¹H NMR (400 MHz, DMSO-d₆) δ 8.67 (d, J=4.7 Hz, 1H), 8.65 (s, 1H), 8.35 (s, 1H), 7.99 (s, 1H), 7.84-7.75 (m, 2H), 7.33-7.63 (m, 7H), 7.22 (s, 1H), 7.07 (s, 1H), 5.79 (t, J=9.5 Hz, 1H), 5.17-5.11 (m, 1H), 4.51-4.43 (m, 2H), 4.36-4.05 (m, 5H), 3.78 (d, J=11.0 Hz, 2H), 3.64-3.59 (m, 1H), 3.29-3.20 (m, 6H), 3.16-3.05 (m, 2H), 2.94-2.80 (m, 1H), 2.75-2.63 (m, 7H), 2.44-2.20 (m, 4H), 2.18-1.95 (m, 2H), 1.93-1.45 (m, 6H), 1.33 (s, 3H), 1.31 (s, 3H), 0.92 (s, 6H), 0.86-0.69 (m, 7H), 0.48-0.42 (m, 2H), 0.36-0.21 (m, 5H).

### Synthesis of Compound A105—(9S,15S,18R,22S)-2-ethyl-18-isopropyl-3-(2-((S)-1-methoxyethyl)-5-(4-methylpiperazin-1-yl)pyridin-3-yl)-5,5-dimethyl-22-phenyl-2,4,5,6,9,10,11,12,15,16,18,19,22,23-tetradecahydro-8H,21H-9,13-epimino-1,29-etheno-15,26-methano-24,28-(metheno)pyrrolo[3,4-l][1,17]dioxa[4,23,26]triazacyclononacosine-8,14,17-trione

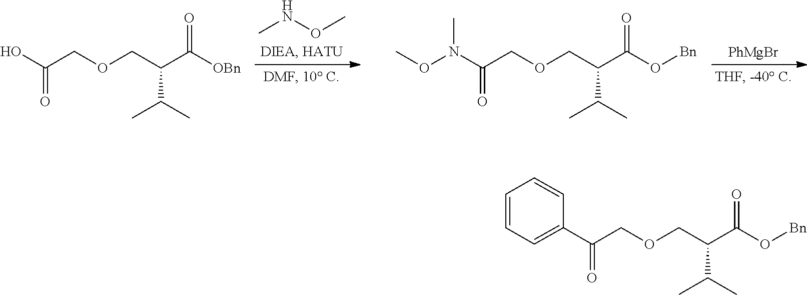

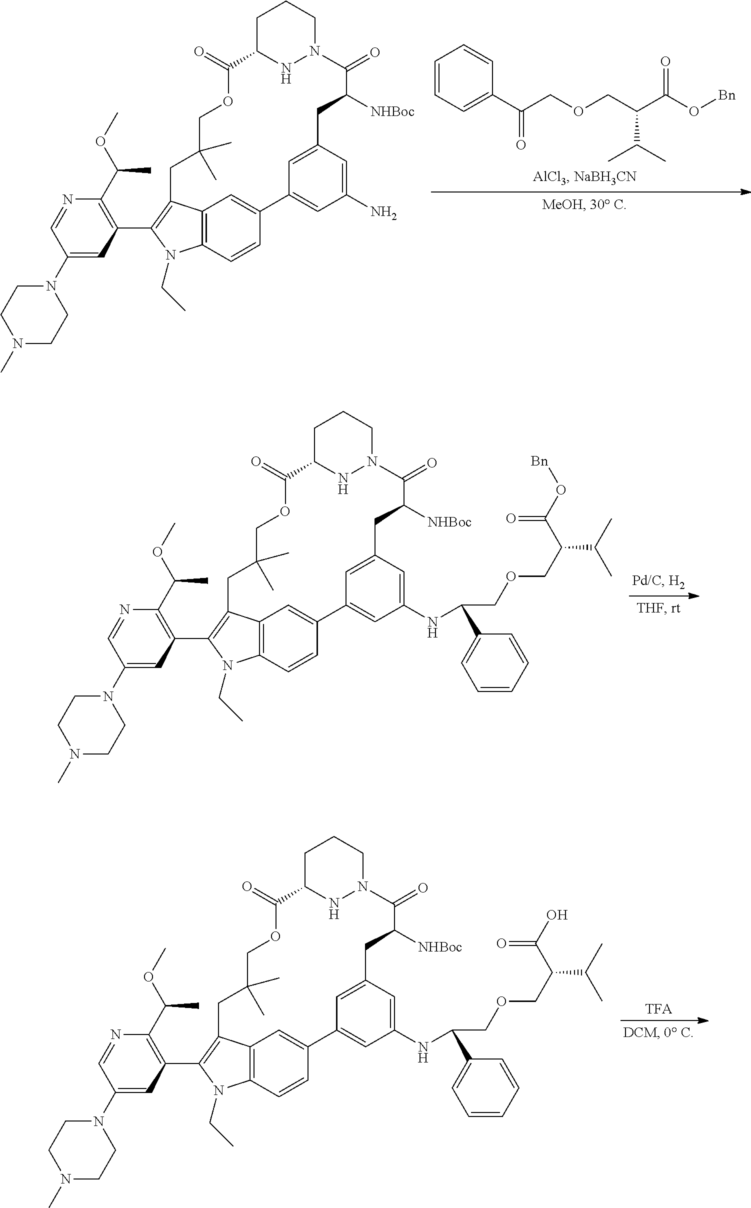

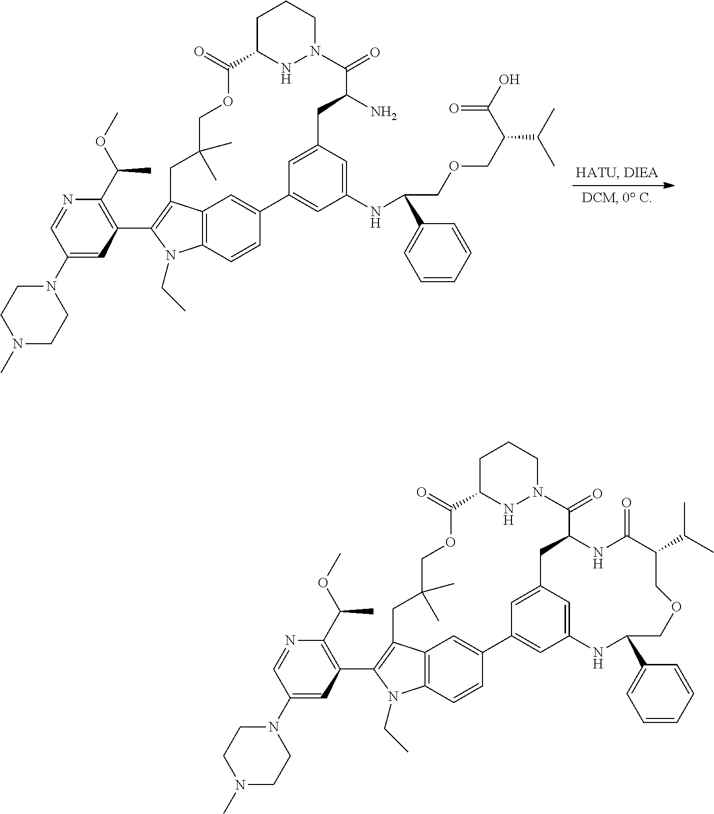

Step 1. To a stirred mixture of N,O-dimethylhydroxylamine (520 mg, 8.51 mmol) in DMF (20 mL), DIEA (14.7 g, 113 mmol), (R)-2-(2-((benzyloxy)carbonyl)-3-methylbutoxy)acetic acid (1.59 g, 5.67 mmol), and HATU (2.16 g, 5.67 mmol) were added at 0° C. The reaction mixture was stirred for 2 hours at 10° C. The resulting mixture was quenched by the addition of sat. aq. NH₄Cl at 0° C., extracted with EtOAc (3×50 mL), treated with brine (3×50 mL), dried over anhydrous Na₂SO₄, filtered, and concentrated under reduced pressure. The residue was purified by normal phase flash column chromatography to afford benzyl (R)-2-((2-(methoxy(methyl)amino)-2-oxoethoxy)methyl)-3-methylbutanoate (1.50 g, 82% yield) as a yellow oil. LCMS (ESI) m/z: [M+H] calcd for C₁₇H₂₅NO₅: 324.2; found 324.2.

Step 2. To a stirred mixture of benzyl (R)-2-((2-(methoxy(methyl)amino)-2-oxoethoxy)methyl)-3-methylbutanoate (500 mg, 1.55 mmol) in THF (5 mL) was added phenylmagnesium bromide (1 M in THF, 2.32 mL, 2.32 mmol) dropwise at −40° C. under an atmosphere of argon. The resulting mixture was stirred for 2 hours at −40° C. The reaction mixture was then quenched by the addition of sat. aq. NH₄Cl at 0° C., extracted with EtOAc (3×25 mL), treated with brine (3×15 mL), dried over anhydrous Na₂SO₄, filtered, concentrated under reduced pressure, and purified by normal phase flash column chromatography to give benzyl (R)-3-methyl-2-((2-oxo-2-phenylethoxy)methyl)butanoate (400 mg, 76% yield) as a dark oil. LCMS (ESI) m/z: [M+H] calcd for C₂₁H₂₄O₄: 341.2; found 341.2.

Step 3. To a stirred mixture of tert-butyl ((63S,4S)-25-amino-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11H-8-oxa-1(5,3)-indola-6(1,3)-pyridazina-2(1,3)-benzenacycloundecaphane-4-yl)carbamate in MeOH (2 mL) were added benzyl (R)-3-methyl-2-((2-oxo-2-phenylethoxy)methyl)butanoate (163 mg, 0.478 mmol) and AlCl₃ (6.37 mg, 0.048 mmol) at 0° C. The resulting mixture was stirred for 1 hour at 0° C., after which time NaBH₃CN (90.1 mg, 1.43 mmol) was added at 0° C. The reaction mixture was stirred for 16 hours at 30° C., after which time it was quenched by the addition of H₂O, extracted with EtOAc (3×30 mL), treated with brine (3×15 mL), dried over anhydrous Na₂SO₄, filtered, concentrated under reduced pressure, and purified by chiral prep-HPLC to yield benzyl (2R)-2-(((2S)-2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-2-phenylethoxy)methyl)-3-methylbutanoate (100 mg, 18% yield, assumed absolute configuration) as a brown-yellow solid (LCMS (ESI) m/z: [M+H] calcd for C₆₃H₈₀N₈O₇: 1161.6; found 1161.6) and benzyl (2R)-2-(((2R)-2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-2-phenylethoxy)methyl)-3-methylbutanoate (100 mg, 18% yield, assumed absolute configuration) as a brown-yellow solid. LCMS (ESI) m/z: [M+H] calcd for C₆₃H₈ON₈O₇: 1161.6; found 1161.6).

Step 4. To a stirred mixture of benzyl (2R)-2-(((2S)-2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)amino)-2-phenylethoxy)methyl)-3-methylbutanoate (100 mg, 0.086 mmol) in THF (1.5 mL) was added 10% Pd/C (45.8 mg) at 0° C. The reaction mixture was stirred for 2 days at room temperature under an atmosphere of H₂. The resulting mixture was then filtered, the filter cake was washed with MeOH (3×5 mL), and the filtrate was concentrated under reduced pressure to give (2R)-2-(((2S)-2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-2-phenylethoxy)methyl)-3-methylbutanoic acid (80.0 mg, 87% yield) as a brown solid. LCMS (ESI) m/z: [M+H] calcd for C₆₁H₈₂N₈O₉: 1171.6; found 1171.6.

Step 5. To a stirred mixture of (2R)-2-(((2S)-2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-2-phenylethoxy)methyl)-3-methylbutanoic acid (80.0 mg, 0.075 mmol) in DCM (2 mL) was added TFA (1 mL) dropwise at 0° C. The reaction mixture was stirred for 2 hour at 0° C. The resulting mixture was diluted with toluene (10 mL) and concentrated under reduced pressure to yield (2R)-2-(((2S)-2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-2-phenylethoxy)methyl)-3-methylbutanoic acid (160 mg, crude) as a brown yellow oil. This material was taken directly to the next reaction without further purification. LCMS (ESI) m/z: [M+H] calcd for C₅₆H₇₄N₈O₇: 971.6; found 971.4.

Step 6. To a stirred mixture of (2R)-2-(((2S)-2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)-2-phenylethoxy)methyl)-3-methylbutanoic acid (70 mg, crude) in DCM (0.5 mL) were added DIEA (186 mg, 1.44 mmol) and HATU (27.4 mg, 0.072 mmol) at 0° C. The resulting mixture was stirred for 2 hours at 0° C. The reaction mixture was quenched by the addition of cold H₂O at 0° C. and was then extracted with EtOAc (3×30 mL), treated with brine (3×10 mL), dried over anhydrous Na₂SO₄, filtered, concentrated under reduced pressure, and purified by reversed-phase flash column chromatography to afford (9S,15S,18R,22S)-2-ethyl-18-isopropyl-3-(2-((S)-1-methoxyethyl)-5-(4-methylpiperazin-1-y)pyridin-3-yl)-5,5-dimethyl-22-phenyl-2,4,5,6,9,10,11,12,15,16,18,19,22,23-tetradecahydro-8H,21H-9,13-epimino-1,29-etheno-15,26-methano-24,28-(metheno)pyrrolo[3,4-l][1,17]dioxa[4,23,26]triazacyclononacosine-8,14,17-trione (2.0 mg, 6.4% yield over 2 steps) as a white solid. LCMS (ESI) m/z: [M+H] calcd for C₅₆H₇₂N₈O₆: 953.6; found 953.6; ¹H NMR (400 MHz, DMSO-d₆) δ 8.45 (d, J=3.0 Hz, 1H), 8.27 (d, J=9.9 Hz, 1H), 7.96 (s, 1H), 7.55-7.48 (m, 2H), 7.44 (d, J=7.7 Hz, 2H), 7.35 (t, J=7.5 Hz, 2H), 7.25 (t, J=7.5 Hz, 1H), 7.21-7.15 (m, 2H), 7.08 (s, 1H), 6.93 (s, 1H), 5.67-5.44 (m, 1H), 5.15 (d, J=11.9 Hz, 1H), 4.38-4.20 (m, 3H), 4.18-3.94 (m, 4H), 3.87-3.69 (m, 2H), 3.68-3.51 (m, 3H), 3.30-3.18 (m, 6H), 3.16-3.08 (m, 3H), 3.04-2.94 (m, 2H), 2.81-2.66 (m, 2H), 2.46 (d, J=6.8 Hz, 4H), 2.34-2.18 (m, 3H), 2.14-2.01 (m, 2H), 1.98-1.41 (m, 5H), 1.42-1.29 (m, 3H), 1.24 (d, J=5.8 Hz, 3H), 1.02-0.63 (m, 13H), 0.43 (d, J=12.8 Hz, 3H).

### Synthesis of Compound A159—(9S,15S,18S,20aR)-2-ethyl-18-isopropyl-3-(2-((S)-1-methoxyethyl)pyridin-3-yl)-5,5,19-trimethyl-2,4,5,6,9,10,11,12,15,16,18,19,20a,21,22,23,26,27-octadecahydro-8H,14H,25H-9,13-epimino-1,33-etheno-15,30-methano-28,32-(metheno)dipyrrolo[3,4-a₁:2′,1′-o][1]oxa[7,10,13,16,19]pentaazacyclohentriacontine-8,14,17,20-tetraone

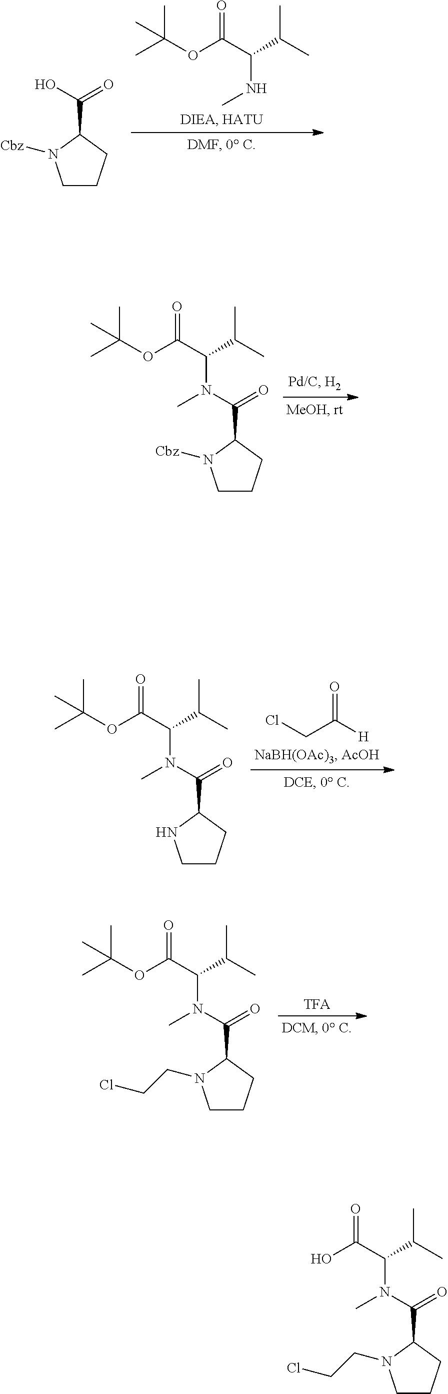

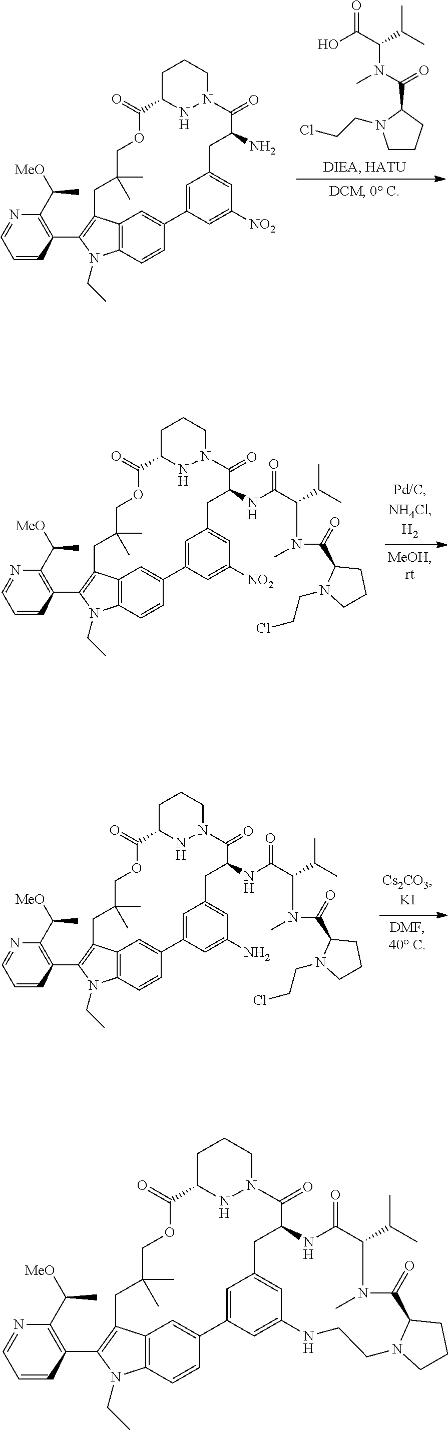

Step 1. To a stirred solution of N-(benzyloxycarbonyl)-D-proline (5.00 g, 20.0 mmol) and tert-butyl methyl-L-valinate (5.60 g, 30.0 mmol) in DMF (90 mL) was added DIEA (7.80 g, 60.0 mmol) followed by HATU (11.4 g, 30.0 mol) at 0° C. The resulting mixture was stirred at 0° C. for 1 hour. The reaction mixture was quenched by the addition of H₂O (100 mL), extracted with EtOAc (3×200 mL), washed with H₂O (200 mL), treated with brine (100 mL), concentrated under reduced pressure, and purified by reversed-phrase flash column chromatography to afford benzyl (R)-2-(((S)-1-(tert-butoxy)-3-methyl-1-oxobutan-2-yl)(methyl)carbamoyl)pyrrolidine-1-carboxylate (10.0 g, 60% yield) as a clear oil. LCMS (ESI): m/z [M+Na] calcd for C₂₃H₃₄N₂O₅ 441.3; found 441.2.

Step 2. To a stirred solution of benzyl (R)-2-(((S)-1-(tert-butoxy)-3-methyl-1-oxobutan-2-yl)(methyl)carbamoyl)pyrrolidine-1-carboxylate (1.00 g, 2.40 mmol) in MeOH (20 mL) was added 10% Pd/C (300 mg) at room temperature under an atmosphere of H₂. The resulting mixture was stirred for 1 hour at room temperature. The reaction mixture was filtered through Celite® and the filtrate was concentrated under reduced pressure, re-dissolved in DCM (20 mL), washed with H₂O (2×5 mL), and again concentrated under reduced pressure to afford tert-butyl N-(D-prolyl)-N-methyl-L-valinate (700 mg, 85% yield) as a clear oil. LCMS (ESI) m/z: [M+H] calcd for C₁₅H₂₈N₂O₃ 285.2; found 285.3.

Step 3. To a stirred solution of tert-butyl N-(D-prolyl)-N-methyl-L-valinate (750 mg, 2.64 mmol) and 2-chloroacetaldehyde (414 mg, 5.28 mmol) in 1,2-DCE (10 mL) was added NaBH(OAc)₃ (2.24 g, 10.6 mmol) followed by AcOH (634 mg, 10.6 mmol) at room temperature. The resulting mixture was stirred for 2 h at room temperature. The reaction mixture was quenched by the addition of H₂O (20 mL), extracted with EtOAc (3×40 mL), washed with H₂O (20 mL), treated with brine (20 mL), and concentrated under reduced pressure to afford tert-butyl N-((2-chloroethyl)-D-prolyl)-N-methyl-L-valinate (900 mg, crude). This material was taken directly to the next reaction without further purification. LCMS (ESI) m/z: [M+H] calcd for C₁₇H₃₁ClN₂O₃ 347.2; found 347.2.

Step 4. A mixture of tert-butyl N-((2-chloroethyl)-D-prolyl)-N-methyl-L-valinate (270 mg, crude) in DCM (3 mL) and TFA (3 mL) was stirred at room temperature for 2 hours. The reaction mixture was then concentrated under reduced pressure to give (2S)-2-{1-[(2R)-1-(2-chloroethyl)pyrrolidin-2-yl]-N-methylformamido}-3-methylbutanoic acid (250 mg) as an oil. This material was taken directly to the next reaction without further purification.

Step 5. To a stirred mixture (6³S,4S)-4-amino-1¹-ethyl-1²-(2-((S)-1-methoxyethyl)pyridin-3-yl)-10,10-dimethyl-2⁵-nitro-6¹,6²,6³,6⁴,6⁵,6⁶-hexahydro-1¹H-8-oxa-1(5,3)-indola-6(1,3)-pyridazina-2(1,3)- benzenacycloundecaphane-5,7-dione (500 mg, 0.7 mmol) and (2S)-2-{1-[(2R)-1-(2-chloroethyl)pyrrolidin-2-yl]-N-methylformamido}-3-methylbutanoic acid (326 mg, 1.1 mmol) in DMF (5 mL) at 0° C. were added DIEA (966 mg, 7.40 mmol) and HATU (426 mg, 1.10 mmol). This mixture was stirred at 0° C. for 2 hours. The reaction mixture was then quenched by the addition of H₂O (10 mL), extracted with EtOAc (3×10 mL), washed with H₂O (10 mL), treated with brine (10 mL), concentrated under reduced pressure, and purified by normal phase prep-TLC to give (2R)-1-(2-chloroethyl)-N-((2S)-1-(((6³S,4S)-1¹-ethyl-1²-(2-((S)-1-methoxyethyl)pyridin-3-yl)-10,10-dimethyl-2⁵-nitro-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-2-carboxamide (500 mg, 63% yield) as a solid. LCMS (ESI) m/z: [M+H] calcd for C₅₀H₆₅ClN₈O₈ 941.5; found 941.4.

Step 6. A mixture of (2R)-1-(2-chloroethyl)-N-((2S)-1-(((6³S,4S)-1¹-ethyl-1²-(2-((S)-1-methoxyethyl)pyridin-3-yl)-10,10-dimethyl-2⁵-nitro-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-2-carboxamide (200 mg, 0.20 mmol), 10% Pd/C (113 mg, 1.10 mmol) and NH₄Cl (227 mg, 4.30 mmol) in MeOH (3 mL) was evacuated and backfilled three times with H₂ gas. This mixture was stirred under an atmosphere of H₂ at room temperature for 24 hours. The resulting mixture was filtered through Celite® and the filtrate was concentrated under reduced pressure to afford (2R)—N-((2S)-1-(((6³S,4S)-2⁵-amino-1¹-ethyl-1²-(2-((S)-1-methoxyethyl)pyridin-3-yl)-10,10-dimethyl-5,7-dioxo-6¹,6²,6³,6⁴,6⁵,6⁶6-hexahydro-1¹H-8-oxa-1(5,3)-indola-6(1,3)-pyridazina-2(1,3)-benzenacycloundecaphane-4-yl)amino)-3-methyl-1-oxobutan-2-yl)-1-(2-chloroethyl)-N-methylpyrrolidine-2-carboxamide (25 mg, 49% yield) as a solid. This material was taken directly to the next reaction without further purification. LCMS (ESI) m/z: [M+H] calcd for C₅₀H₆₇ClN₈O₆ 911.5; found 911.4.

Step 7. To a stirred solution of (2R)—N-((2S)-1-(((6³S,4S)-2⁵-amino-1¹-ethyl-1²-(2-((S)-1-methoxyethyl)pyridin-3-yl)-10,10-dimethyl-5,7-dioxo-6¹,6²,6³,6⁴,6⁵,6⁶6-hexahydro-1¹H-8-oxa-1(5,3)-indola-6(1,3)-pyridazina-2(1,3)-benzenacycloundecaphane-4-yl)amino)-3-methyl-1-oxobutan-2-yl)-1-(2-chloroethyl)-N-methylpyrrolidine-2-carboxamide (200 mg, 0.20 mmol) in DMF (6 mL) at 40° C. were added Cs₂CO₃ (143 mg, 0.40 mmol) and KI (73 mg, 0.40 mmol). The resulting mixture was stirred at 40° C. for 16 hours. The reaction mixture was then diluted with EtOAc (10 mL), washed with H₂O (3×10 mL), treated with brine (10 mL), dried over anhydrous Na₂SO₄, filtered, concentrated under reduced pressure, and purified by reversed-phase prep-HPLC to afford (9S,15S,18S,20aR)-2-ethyl-18-isopropyl-3-(2-((S)-1-methoxyethyl)pyridin-3-yl)-5,5,19-trimethyl-2,4,5,6,9,10,11,12,15,16,18,19,20a,21,22,23,26,27-octadecahydro-8H,14H,25H-9,13-epimino-1,33-etheno-15,30-methano-28,32-(metheno)dipyrrolo[3,4-a₁:2′,1′-o][1]oxa[7,10,13,16,19]pentaazacyclohentriacontine-8,14,17,20-tetraone (20 mg, 10% yield) as a white solid. LCMS (ESI) m/z: [M+H] calcd for C₅₀H₆₆N₈O₆ 875.5; found 875.4; ¹H NMR (400 MHz, CD₃OD) δ 8.72 (dd, J=4.8, 1.7 Hz, 1H), 8.03 (d, J=25.2 Hz, 1H), 7.85 (dd, J=7.7, 1.7 Hz, 1H), 7.67 (dd, J=8.8, 1.4 Hz, 1H), 7.57-7.43 (m, 2H), 7.20 (s, 1H), 7.04 (s, 1H), 6.55 (s, 1H), 5.44-5.30 (m, 1H), 4.94 (d, J=12.5 Hz, 1H), 4.83 (s, 1H), 4.46 (d, J=12.3 Hz, 1H), 4.36-4.31 (m, 1H), 4.23 (dd, J=14.5, 7.3 Hz, 1H), 4.20-4.01 (m, 2H), 3.84 (d, J=11.0 Hz, 1H), 3.79-3.63 (m, 2H), 3.44-3.39 (m, 1H), 3.29-3.22 (m, 4H), 3.07 (s, 1H), 2.96 (s, 3H), 2.85 (d, J=9.4 Hz, 2H), 2.82-2.61 (m, 4H), 2.49 (s, 1H), 2.22-2.14 (m, 3H), 1.95 (d, J=14.6 Hz, 3H), 1.82 (d, J=13.2 Hz, 1H), 1.77-1.60 (m, 2H), 1.45 (t, J=6.5 Hz, 3H), 1.08-0.92 (m, 6H), 0.85-0.71 (m, 6H), 0.56 (s 3H).

### Synthesis of Compound A133—(9S,15S,17aR,18S,18aR)-2-ethyl-3-(2-((S)-1-methoxyethyl)-5-(4-methylpiperazin-1-yl)pyridin-3-yl)-5,5,18-trimethyl-2,4,5,6,9,10,11,12,15,16,17a,18,18a,19,22,23-hexadecahydro-8H,21H-9,13-epimino-1,29-etheno-15,26-methano-24,28-(metheno)cyclopropa[b₁]pyrrolo[3,4-l][1,17]dioxa[4,23,26]triazacyclotriacontine-8,14,17-trione

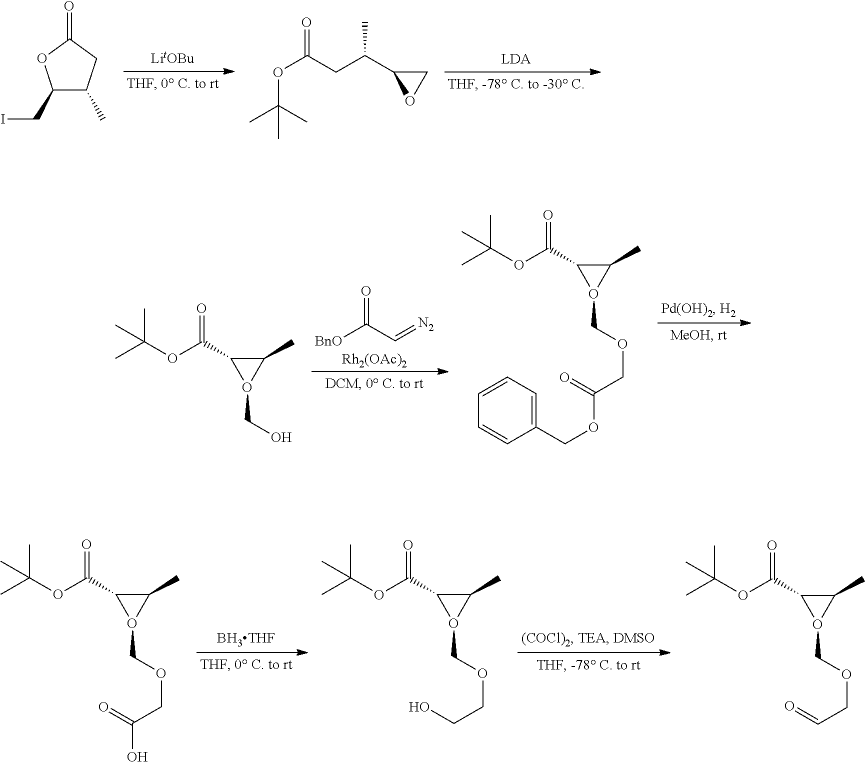

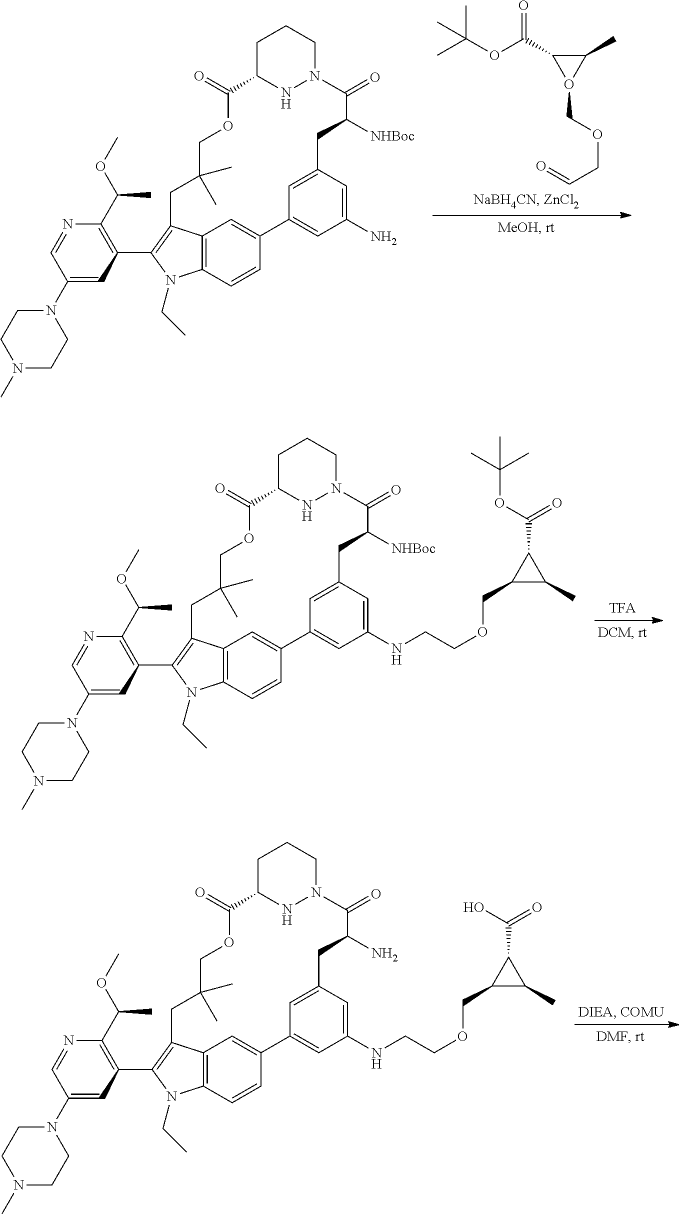

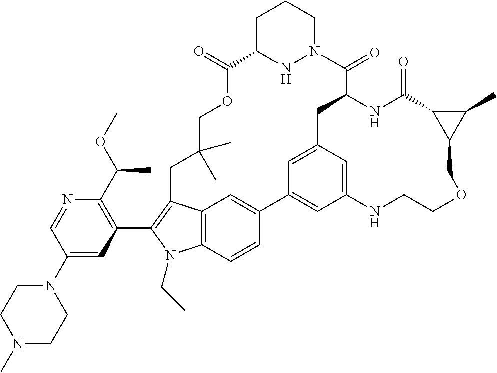

Step 1. A solution of trans-5-(iodomethyl)-4-methyloxolan-2-one (480 g, 2.0 mol, racemic mixture) and lithium tert-butoxide (240 g, 3.0 mol) in THF (5.0 L) was stirred for 1 hour at 0° C. under an atmosphere of N₂ and was then stirred for 18 hours at room temperature. The reaction mixture was quenched by the addition of sat. aq. NH₄Cl (5 L) at 0° C., extracted with EtOAc (2×5 L), washed with H₂O (5 L), dried over anhydrous Na₂SO₄, filtered, and concentrated under reduced pressure to give tert-butyl 3-(oxiran-2-yl)butanoate (340 g, crude) as a yellow oil. This material was taken directly to the next reaction without further purification. ¹H NMR (400 MHz, DMSO-d₆) δ 2.81-2.78 (m, 1H), 2.69-2.64 (m, 1H), 2.53-2.47 (m, 1H), 2.36-2.33 (m, 1H), 2.17-2.08 (m, 1H), 1.71-1.69 (m, 1H), 1.40 (s, 9H), 0.95-0.93 (m, 3H).

Step 2. To a stirred solution of tert-butyl 3-(oxiran-2-yl)butanoate (340 g, crude) in THF (5 L) was added LDA (2.0 M in THF, 1.0 L, 2.00 mol) dropwise at −78° C. under an atmosphere of N₂. The resulting mixture was stirred for 1 hour at −78° C. and was then stirred at −30° C. for 3 hours under an atmosphere of N₂. The reaction mixture was quenched by the addition of sat. aq. NH₄Cl (3 L) at 0° C., extracted with EtOAc (3×3 L), washed with H₂O (1 L), dried over anhydrous Na₂SO₄, filtered, concentrated under reduced pressure, and purified by normal phase flash column chromatography to afford tert-butyl 2-(hydroxymethyl)-3-methylcyclopropane-1-carboxylate (160 g, racemic mixture) as a yellow oil. This racemic mixture was resolved by prep-SFC-HPLC to afford tert-butyl (1R,2R,3S)-2-(hydroxymethyl)-3-methylcyclopropane-1-carboxylate (72.0 g, 19% yield over 2 steps, assumed absolute configuration). ¹H NMR (400 MHz, Chloroform-d) δ 3.78-3.75 (m, 1H), 3.59-3.57 (m, 1H), 1.89-1.71 (m, 1H), 1.70-1.68 (m, 1H), 1.52-1.50 (m, 1H), 1.43 (s, 9H), 1.18-1.15 (m, 3H).

Step 3. To a stirred mixture of Rh₂(OAc)₄ (59.3 mg, 0.134 mmol) in DCM (10 mL) was added tert-butyl (1R,2R,3S)-2-(hydroxymethyl)-3-methylcyclopropane-1-carboxylate (500 mg, 2.69 mmol) in portions over 5 minutes at room temperature under an atmosphere of N₂. Subsequently, benzyl 2-diazoacetate (946 mg, 5.37 mmol) was added dropwise at 0° C. and the resulting mixture was stirred for 18 hours at room temperature under an atmosphere of N₂. The reaction mixture was filtered, the filter cake was washed with DCM (3×10 mL), the filtrate was concentrated under reduced pressure, and the resulting residue was purified by normal phase flash column chromatography to afford tert-butyl (1R,2R,3S)-2-((2-(benzyloxy)-2-oxoethoxy)methyl)-3-methylcyclopropane-1-carboxylate (430 mg, 48% yield) as yellow oil. ¹H NMR (400 MHz, Chloroform-d) δ 7.36-7.24 (m, 5H), 5.19 (s, 2H), 4.14 (m, 2H), 3.75-3.42 (m, 2H), 1.73-1.70 (m, 1H), 1.58-1.51 (m, 1H), 1.43 (s, 9H), 1.20-1.15 (m, 1H), 1.13-1.10 (m, 3H).

Step 4. A solution of tert-butyl (1R,2R,3S)-2-((2-(benzyloxy)-2-oxoethoxy)methyl)-3-methylcyclopropane-1-carboxylate (430 mg, 1.29 mmol) and Pd(OH)₂/C (430 mg, 3.06 mmol) in MeOH (5 mL) was stirred for 1.5 hours at room temperature under an atmosphere of H₂. The reaction mixture was filtered, the filter cake was washed with MeOH (3×10 mL), and the filtrate was concentrated under reduced pressure to give 2-(((1R,2R,3S)-2-(tert-butoxycarbonyl)-3-methylcyclopropyl)methoxy)acetic acid (270 mg, crude) as a dark solid. This material was taken directly to the next reaction without further purification. ¹H NMR (400 MHz, Chloroform-d) δ 4.14-5.10 (m, 2H), 3.78-3.65 (m, 1H), 3.55-3.51 (m, 1H), 1.73-1.70 (m, 1H), 1.59-1.50 (m, 1H), 1.44 (s, 9H), 1.20-1.15 (m, 1H), 1.16-1.24 (m, 3H).

Step 5. 2-(((1R,2R,3S)-2-(tert-butoxycarbonyl)-3-methylcyclopropyl)methoxy)acetic acid (250 mg, crude) was added to BH₃·THF (0.29 mL, 3.1 mmol) at 0° C. The resulting mixture was stirred for 18 hours at room temperature under an atmosphere of N₂. The reaction mixture was quenched by the addition of MeOH (20 mL) at 0° C., concentrated under reduced pressure, and purified by normal phase flash column chromatography to afford tert-butyl (1R,2R,3S)-2-((2-hydroxyethoxy)methyl)-3-methylcyclopropane-1-carboxylate (140 mg, 51% yield over 2 steps) as light yellow oil. ¹H NMR (300 MHz, Chloroform-d) δ 3.79-3.73 (m, 2H), 3.68-3.65 (m, 1H), 3.63-3.52 (m, 2H), 3.44-3.42 (m, 1H), 1.72-1.70 (m, 1H), 1.60-1.49 (m, 1H), 1.46 (s, 9H), 1.22-1.18 (m, 1H), 1.17-1.12 (m, 3H).

Step 6. To a stirred solution of oxalyl chloride (386 mg, 3.04 mmol) in DCM (5 mL) was added DMSO (475 mg, 6.08 mmol) dropwise at −78° C. under an atmosphere of N₂. To this mixture was added tert-butyl (1R,2R,3S)-2-[(2-hydroxyethoxy)methyl]-3-methylcyclopropane-1-carboxylate (350 mg, 1.52 mmol) dropwise over 30 minutes at −78° C. Subsequently, TEA (923 mg, 9.12 mmol) was added dropwise at −78° C. and the resulting mixture was stirred for 1 hour at room temperature. The reaction was quenched by the addition H₂O (50 mL), extracted with EtOAc (3×50 mL), dried over anhydrous Na₂SO₄, filtered, and concentrated under reduced pressure to give tert-butyl (1R,2S,3R)-2-methyl-3-[(2-oxoethoxy)methyl]cyclopropane-1-carboxylate (280 mg, crude) as a yellow oil. This material was taken directly to the next reaction without further purification.

Step 7. To a stirred solution of (6³S,4S)-25,4-diamino-1¹-ethyl-1²-(2-((S)-1-methoxyethyl)-5-(4-methylpiperazin-1-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (380 mg, 0.516 mmol) in MeOH (5 mL) were added tert-butyl (1R,2S,3R)-2-methyl-3-[(2-oxoethoxy)methyl]cyclopropane-1-carboxylate (235 mg, crude) and ZnCl₂ (70.3 mg, 0.516 mmol) at room temperature. Subsequently, NaBH₃CN (162 mg, 2.58 mmol) was added at 0° C. and the resulting mixture was stirred for 1 hour at room temperature. The reaction mixture was quenched with H₂O (30 mL) at room temperature, extracted with EtOAc (3×30 mL), dried over anhydrous Na₂SO₄, filtered, concentrated under reduced pressure, and purified by normal phase flash column chromatography to afford tert-butyl (1R,2R,3S)-2-((2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethoxy)methyl)-3-methylcyclopropane-1-carboxylate (306 mg, 57% yield) as a yellow oil. LCMS (ESI) m/z: [M+H] calcd for C₅₉H₈₄N₈O₉: 1049.6; found 1049.5.

Step 8. To a stirred solution of tert-butyl (1R,2R,3S)-2-((2-(((6³S,4S)-4-((tert-butoxycarbonyl)amino)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethoxy)methyl)-3-methylcyclopropane-1-carboxylate (300 mg, 0.286 mmol) in DCM (10 mL) was added TFA (5.0 mL). The resulting mixture was stirred for 1 hour at room temperature and was then concentrated under reduced pressure to give (1R,2R,3S)-2-((2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethoxy)methyl)-3-methylcyclopropane-1-carboxylic acid (320 mg, crude) as a yellow solid. This material was taken directly to the next reaction without further purification. LCMS (ESI) m/z: [M+H] calcd for C₅₀H₆₈N₈O₇: 893.5; found 893.6.

Step 9. To a stirred solution of (1R,2R,3S)-2-((2-(((6³S,4S)-4-amino-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethoxy)methyl)-3-methylcyclopropane-1-carboxylic acid (320 mg, crude) in DMF (30 mL) were added DIEA (740 mg, 5.73 mmol) and COMU (135 mg, 0.315 mmol) at room temperature. The resulting mixture was stirred for 1 hour at room temperature. The reaction mixture was then diluted with H₂O (100 mL), extracted with EtOAc (3×20 mL), filtered, concentrated under reduced pressure, and purified by reversed-phase prep-HPLC to afford (9S,15S,17aR,18S,18aR)-2-ethyl-3-(2-((S)-1-methoxyethyl)-5-(4-methylpiperazin-1-yl)pyridin-3-yl)-5,5,18-trimethyl-2,4,5,6,9,10,11,12,15,16,17a,18,18a,19,22,23-hexadecahydro-8H,21H-9,13-epimino-1,29-etheno-15,26-methano-24,28-(metheno)cyclopropa[b₁]pyrrolo[3,4-l][1,17]dioxa[4,23,26]triazacyclotriacontine-8,14,17-trione (35.1 mg, 14% yield over 2 steps) as a white solid. LCMS (ESI) m/z: [M+H] calcd for C₅₀H₆₆N₈O₆: 875.5; found 875.8; ¹H NMR (400 MHz, DMSO-d₆) δ 8.44 (d, J=8.8 Hz, 1H), 8.38 (s, 1H), 7.95 (s, 1H), 7.51-7.48 (m, 2H), 7.13 (d, J=2.8 Hz, 1H), 6.90-6.86 (m, 2H), 6.45 (br s, 1H), 5.77-5.68 (m, 1H), 5.33-5.29 (m, 1H), 5.07 (d, J=12.4 Hz, 1H), 4.24-4.13 (m, 2H), 4.13-3.98 (m, 3H), 3.79-3.63 (m, 2H), 3.56-3.45 (m, 3H), 3.19 (s, 3H), 3.12 (s, 3H), 3.10-3.02 (m, 1H), 2.95-2.84 (m, 1H), 2.76-2.57 (m, 3H), 2.41-2.33 (m, 5H), 2.15 (s, 3H), 2.03-1.92 (m, 1H), 1.82-1.64 (m, 2H), 1.56-1.41 (m, 2H), 1.36-1.12 (m, 6H), 1.11-0.95 (m, 4H), 0.88-0.74 (m, 7H), 0.32 (s, 3H).

### Synthesis of Compound A163—(9S,15S,18S,Z)-2-ethyl-18-isopropyl-3-(2-(methoxymethyl)pyridin-3-yl)-5,5,19-trimethyl-2,4,5,6,9,10,11,12,15,16,18,19-dodecahydro-8H,14H,24H-9,13-epimino-1,30-etheno-15,27-methano-25,29-(metheno)pyrrolo[3,4-z][1]oxa[17]thia[7,10,13,18]tetraazacyclotriacontine-8,14,17,20-tetraone 23,23-dioxide

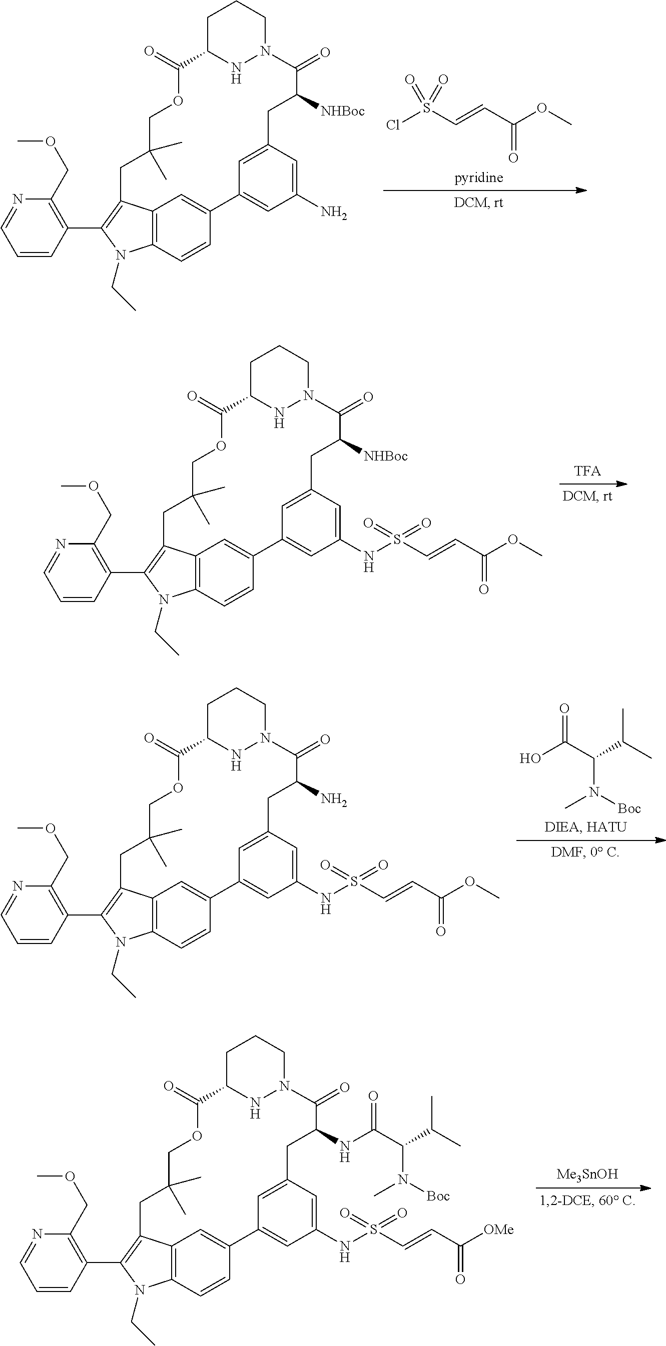

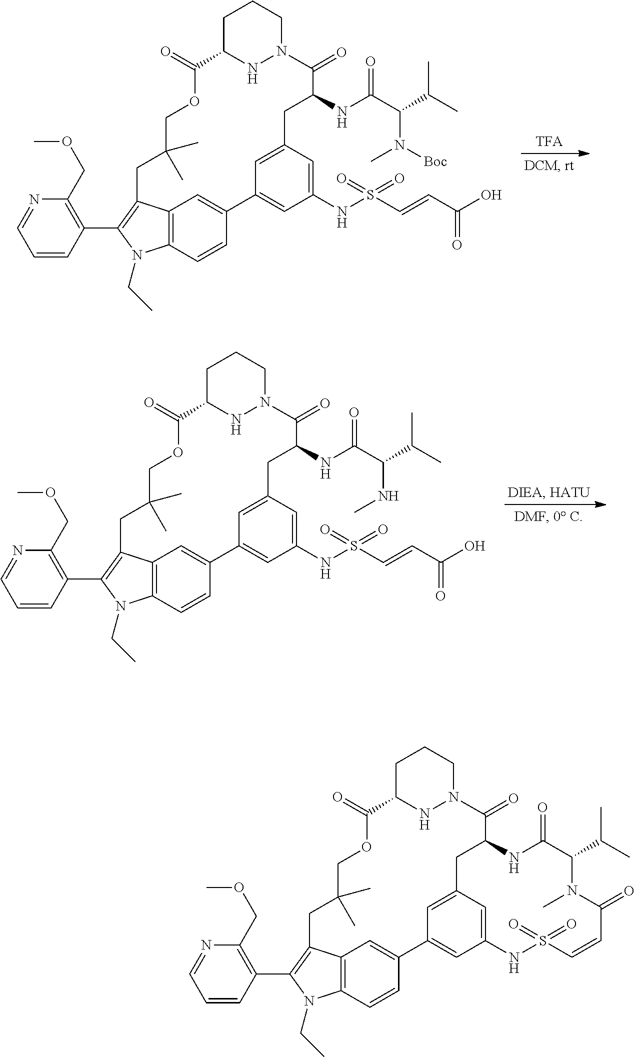 [SMILES: CCn1c(-c2cccnc2COC)c2c3cc(ccc31)-c1cc(cc(NS(=O)(=O)/C=C/C(=O)O)c1)C[C@H](NC(=O)[C@H](C(C)C)N(C)C(=O)OC(C)(C)C)C(=O)N1CCC[C@H](N1)C(=O)OCC(C)(C)C2 ; CCn1c(-c2cccnc2COC)c2c3cc(ccc31)-c1cc(cc(NS(=O)(=O)/C=C/C(=O)O)c1)C[C@H](NC(=O)[C@@H](NC)C(C)C)C(=O)N1CCC[C@H](N1)C(=O)OCC(C)(C)C2 ; "DIEA, HATU" ; "TFA" ; CCn1c(-c2cccnc2COC)c2c3cc(ccc31)-c1cc3cc(c1)NS(=O)(=O)/C=C\C(=O)N(C)[C@@H](C(C)C)C(=O)N[C@@H](C3)C(=O)N1CCC[C@H](N1)C(=O)OCC(C)(C)C2]

Step 1. To a solution of tert-butyl ((6³S,4S)-2⁵-amino-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)carbamate (280 mg, 0.39 mmol) and methyl (2E)-3-(chlorosulfonyl)prop-2-enoate (107 mg, 0.58 mmol) in DCM (5 mL) stirred at room temperature was added pyridine (91.7 mg, 1.16 mmol). The resulting solution was stirred for 16 hours at room temperature. The reaction mixture was diluted with DCM (20 mL), washed with water (4×10 mL), treated with brine (10 mL), dried over anhydrous Na₂SO₄, filtered, concentrated under reduced pressure, and purified by normal phase flash column chromatography to methyl (E)-3-(N-((6³S,4S)-4-((tert-butoxycarbonyl)amino)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylate (150 mg, 40% yield) as yellow solid. LCMS (ESI) m/z: [M+H] calcd for C₄₅H₅₆N₆O₁₀S: 873.4; found 873.3.

Step 2. Methyl (E)-3-(N-((6³S,4S)-4-((tert-butoxycarbonyl)amino)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylate (180 mg, 0.18 mmol) was dissolved in a mixture of DCM (4 mL) and TFA (0.3 mL). The resulting mixture was stirred for 2 hours at room temperature and was then concentrated under reduced pressure to afford methyl (E)-3-(N-((6³S,4S)-4-amino-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-2⁵-yl)sulfamoyl)acrylate (150 mg, crude) as white solid. This material was taken directly to the next reaction without further purification. LCMS (ESI) m/z: [M+H] calcd for C₄₀H₄₈N₆O₈S: 773.3; found 773.4.

Step 3. To a stirred solution of N-(tert-butoxycarbonyl)-N-methyl-L-valine (67.4 mg, 0.29 mmol), methyl (E)-3-(N-((6³S,4S)-4-amino-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylate (150 mg, crude), and DIEA (250 mg, 1.00 mmol) in DMF (3 mL) was added HATU (88 mg, 0.23 mmol) at 0° C. The resulting mixture was stirred at 0° C. for 1 hour. The reaction mixture was then poured into H₂O (150 mL), extracted with EtOAc (2×150 mL), washed with H₂O (110 mL), treated with brine (50 mL), dried over anhydrous Na₂SO₄, filtered, concentrated under reduced pressure, and purified by normal-phase prep-TLC to afford methyl (E)-3-(N-((6³S,4S)-4-((S)-2-((tert-butoxycarbonyl)(methyl)amino)-3-methylbutanamido)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylate (145 mg, 82% yield over 2 steps) as white solid. LCMS (ESI) m/z: [M+H] calcd for C₅₁H₆₇N₇O₁₁S: 986.5; found 986.5.

Step 4. To a stirred solution of methyl (E)-3-(N-((6³S,4S)-4-((S)-2-((tert-butoxycarbonyl)(methyl)amino)-3-methylbutanamido)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylate (145 mg, 0.15 mmol) in 1,2-DCE (4 mL) was added trimethyltin hydroxide (213 mg, 1.20 mmol). The resulting mixture was stirred at 60° C. for 8 hours and was then filtered and concentrated to afford (E)-3-(N-((6³S,4S)-4-((S)-2-((tert-butoxycarbonyl)(methyl)amino)-3-methylbutanamido)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylic acid (140 mg, crude) as white solid. This material was taken directly to the next reaction without further purification. LCMS (ESI) m/z: [M+H] calcd for C₅₀H₆₅N₇O₁₁S: 972.5; found 972.4.

Step 5. (E)-3-(N-((6³S,4S)-4-((S)-2-((tert-butoxycarbonyl)(methyl)amino)-3-methylbutanamido)-1¹-ethyl-1²-(2-(methoxymethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylic acid (140 mg, crude) was dissolved in a mixture of DCM (4 mL) and TFA (0.3 mL). The resulting mixture was then stirred at room temperature for 2 hours and concentrated under reduced pressure to afford (E)-3-(N-((6³S,4S)-1¹-ethyl-1²-(2-(methoxymethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylic acid (100 mg, crude) as yellow solid. This material was taken directly to the next reaction without further purification. LCMS (ESI) m/z: [M+H] calcd for C₄₅H₅₇N₇O₉S: 872.4; found 872.3.

Step 6. To a stirred solution of (E)-3-(N-((6³S,4S)-1¹-ethyl-1²-(2-(methoxymethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)sulfamoyl)acrylic acid (100 mg, crude) and DIEA (129 mg, 1.00 mmol) in DMF (3 mL) was added HATU (57.0 mg, 0.15 mmol) at 0° C. The resulting mixture was stirred at 0° C. for 1 hour. The reaction mixture was then poured into H₂O (150 mL), extracted with EtOAc (2×150 mL), washed with H₂O (110 mL), treated with brine (50 mL), dried over anhydrous Na₂SO₄, filtered, concentrated under reduced pressure, and purified by normal phase prep-TLC to afford (9S,15S,18S,Z)-2-ethyl-18-isopropyl-3-(2-(methoxymethyl)pyridin-3-yl)-5,5,19-trimethyl-2,4,5,6,9,10,11,12,15,16,18,19-dodecahydro-8H,14H,24H-9,13-epimino-1,30-etheno-15,27-methano-25,29-(metheno)pyrrolo[3,4-z][1]oxa[17]thia[7,10,13,18]tetraazacyclotriacontine-8,14,17,20-tetraone 23,23-dioxide (60.0 mg, 47% yield over 3 steps) as white solid. LCMS (ESI) m/z: [M+H] calcd for C₄₅H₅₅N₇O₈S: 854.4; found 854.4. ¹H NMR (400 MHz, MeOD): δ 8.72-8.66 (m, 2H), 8.07 (d, J=6.5 Hz, 1H), 7.96-7.85 (m, 1H), 7.84-7.63 (m, 1H), 7.58-7.42 (m, 3H), 7.41-7.32 (m, 1H), 7.28-7.24 (m, 1H), 7.20-7.14 (m, 1H), 5.31-5.21 (m, 1H), 4.45-4.28 (m, 2H), 4.26-4.16 (m, 2H), 4.15-4.08 (m, 1H), 3.94-3.86 (m, 1H), 3.83-3.76 (m, 1H), 3.74-3.66 (m, 1H), 3.30 (s, 1H), 3.15 (s, 2H), 3.14-3.07 (m, 3H), 3.05-2.93 (m, 2H), 2.83-2.61 (m, 3H), 2.31-2.16 (m, 3H), 2.01-1.47 (m, 4H), 1.28 (s, 1H), 1.22-1.16 (m, 4H), 1.06-0.98 (m, 4H), 0.9-0.78 (m, 3H), 0.47-0.45 (m, 3H).

### Synthesis of Compound A89—(9S,15S,18R)-3-(5-(4-cyclopropylpiperazin-1-yl)-2-((S)-1-methoxyethyl)pyridin-3-yl)-2-ethyl-18-isopropyl-5,5-dimethyl-2,4,5,6,9,10,11,12,15,16,18,19,22,23-tetradecahydro-8H,21H-9,13-epimino-1,29-etheno-15,26-methano-24,28-(metheno)pyrrolo[3,4-r][1,14]dioxa[5,8,23]triazacyclononacosine-8,14,17-trione

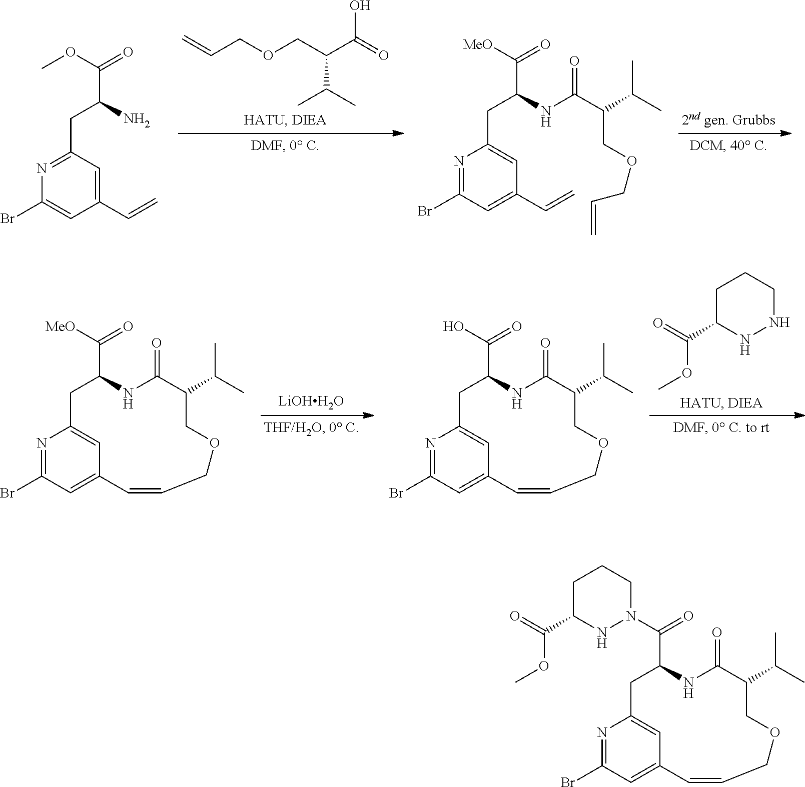

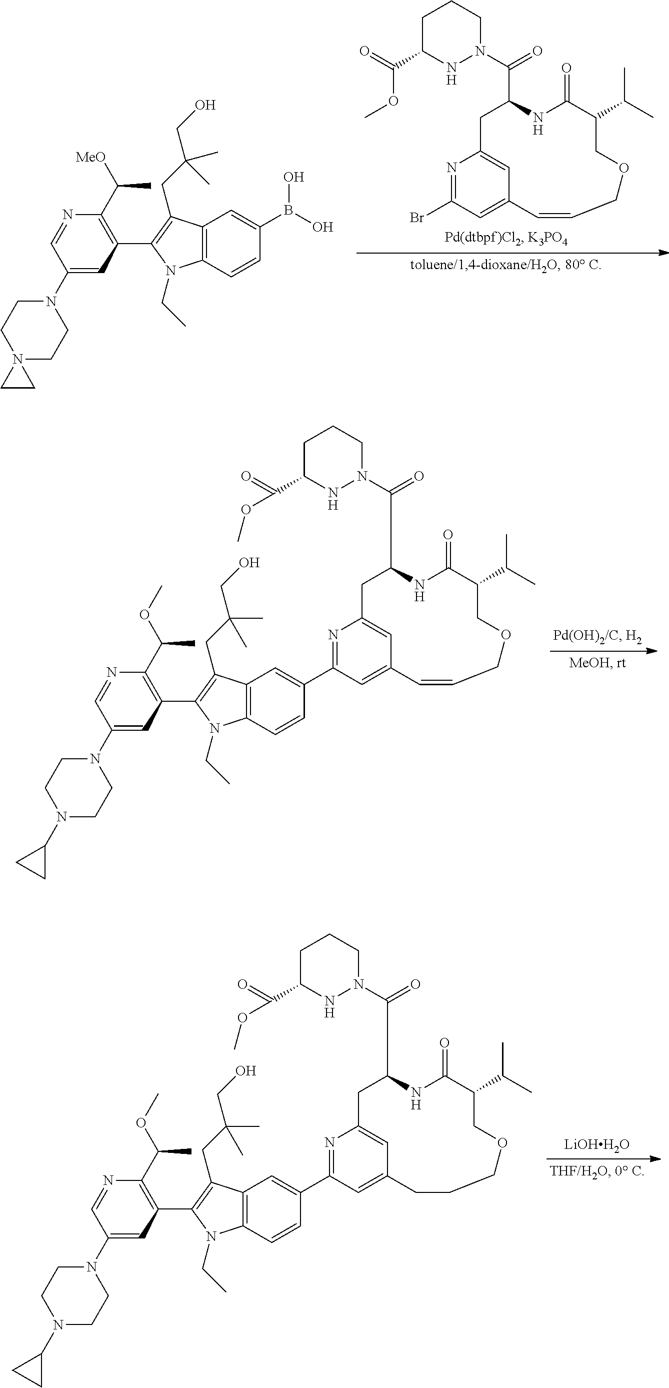

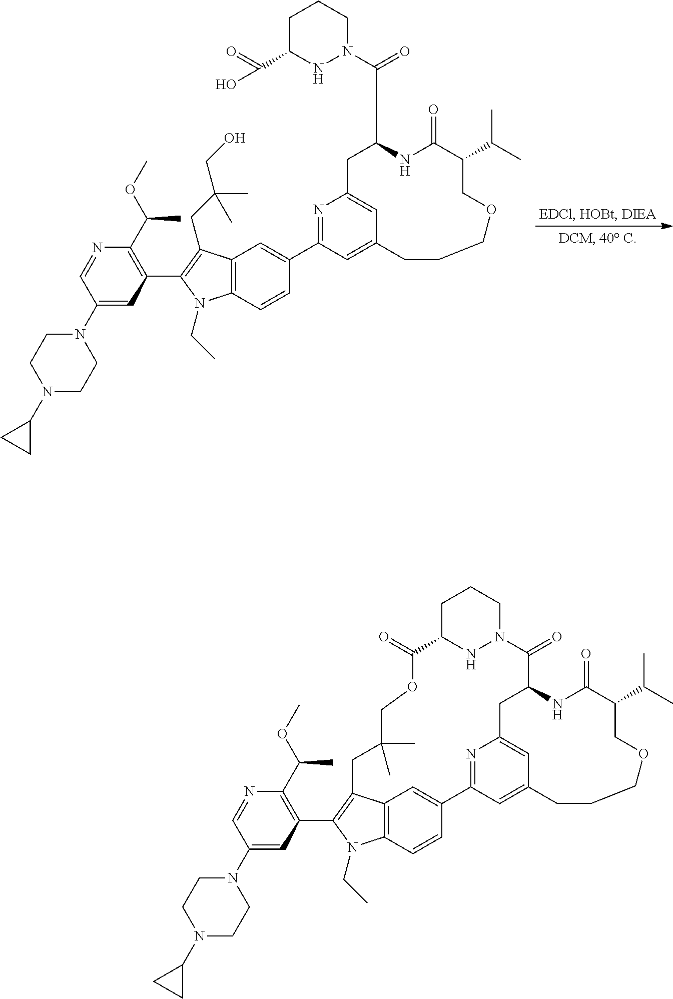

Step 1. To a stirred solution of methyl (S)-2-amino-3-(6-bromo-4-vinylpyridin-2-yl)propanoate (510 mg, 1.79 mmol), (2R)-3-methyl-2-[(prop-2-en-1-yloxy)methyl]butanoic acid (400 mg, 2.33 mmol) and DIEA (9.25 g, 71.6 mmol) in DMF (5 mL) was added HATU (1.02 g, 2.68 mmol) dropwise at 0° C. The resulting mixture was stirred for 2 h at 0° C. The reaction mixture was then quenched by the addition of H₂O (20 mL) at 0° C., extracted with EtOAc (3×20 mL), treated with brine (3×10 mL), dried over anhydrous Na₂SO₄, filtered, concentrated under reduced pressure, and purified by normal phase flash column chromatography to afford methyl (S)-2-((R)-2-((allyloxy)methyl)-3-methylbutanamido)-3-(6-bromo-4-vinylpyridin-2-yl)propanoate (500 mg, 64% yield) as a white solid. LCMS (ESI) m/z: [M+H] calcd for C₂₀H₂₇BrN₂O₄: 439.1; found 439.1.

Step 2. A mixture of methyl (S)-2-((R)-2-((allyloxy)methyl)-3-methylbutanamido)-3-(6-bromo-4-vinylpyridin-2-yl)propanoate (300 mg, 0.683 mmol) and 2^{nd} generation Grubbs catalyst (58.0 mg, 0.068 mmol) was stirred in DCM (300 mL) under reflux for 3 h in an atmosphere of N₂. The resulting mixture was concentrated under reduced pressure and purified by normal phase flash column chromatography to afford methyl (3S,6R,Z)-1⁶-bromo-6-isopropyl-5-oxo-8-oxa-4-aza-1(2,4)-pyridinacycloundecaphan-10-ene-3-carboxylate (220 mg, 81% yield) as a white solid. LCMS (ESI) m/z: [M+H] calcd for C₁₈H₂₃BrN₂O₄: 411.1; found 411.1.

Step 3. To a stirred solution of methyl (3S,6R,Z)-1⁶-bromo-6-isopropyl-5-oxo-8-oxa-4-aza-1(2,4)-pyridinacycloundecaphan-10-ene-3-carboxylate (220 mg, 0.535 mmol) in THF (5 mL) was added a solution of LiOH·H₂O (44.9 mg, 1.07 mmol) in H₂O (5 mL) dropwise at 0° C. The resulting mixture was stirred for 2 h at 0° C. The reaction mixture was then acidified to pH 4 with 1 M aq. HCl, extracted with DCM (3×5 mL), dried over anhydrous Na₂SO₄, filtered, and concentrated under reduced pressure to give (3S,6R,Z)-1⁶-bromo-6-isopropyl-5-oxo-8-oxa-4-aza-1(2,4)-pyridinacycloundecaphan-10-ene-3-carboxylic acid (220 mg, crude) as a white solid. This material was taken directly to the next reaction without further purification. LCMS (ESI) m/z: [M+H] calcd for C₁₇H₂₁BrN₂O₄: 397.1; found 396.9.

Step 4. To a stirred solution of (3S,6R,Z)-1⁶-bromo-6-isopropyl-5-oxo-8-oxa-4-aza-1(2,4)-pyridinacycloundecaphan-10-ene-3-carboxylic acid (220 mg, crude), methyl (S)-hexahydropyridazine-3-carboxylate (160 mg, 1.11 mmol, bis-TFA salt), and DIEA (716 mg, 5.54 mmol) in DMF (4 mL) was added a solution of HATU (379 mg, 0.997 mmol) in DMF (1 mL) dropwise at 0° C. The resulting mixture was stirred for 2 h at room temperature. The reaction mixture was then quenched by the addition of H₂O (10 mL) at 0° C., extracted with EtOAc (3×10 mL), treated with brine (2×10 mL), dried over anhydrous Na₂SO₄, filtered, concentrated under reduced pressure, and purified by normal phase flash column chromatography to afford methyl (S)-1-((3S,6R,Z)-1⁶-bromo-6-isopropyl-5-oxo-8-oxa-4-aza-1(2,4)-pyridinacycloundecaphan-10-ene-3-carbonyl)hexahydropyridazine-3-carboxylate (257 mg, 92% yield over 2 steps) as a white solid. LCMS (ESI) m/z: [M+H] calcd for C₂₃H₃₁BrN₄O₅: 523.2; found 523.2.

Step 5. To a stirred mixture of methyl (S)-1-((3S,6R,Z)-1⁶-bromo-6-isopropyl-5-oxo-8-oxa-4-aza-1(2,4)-pyridinacycloundecaphan-10-ene-3-carbonyl)hexahydropyridazine-3-carboxylate (240 mg, 0.459 mmol), Pd(dtbpf)Cl₂ (29.9 mg, 0.046 mmol) and K₃PO₄ (292 mg, 1.38 mmol) in 1,4-dioxane (1 mL), toluene (3 mL) and H₂O (1 mL) was added (S)-(2-(5-(4-cyclopropylpiperazin-1-yl)-2-(1-methoxyethyl)pyridin-3-yl)-1-ethyl-3-(3-hydroxy-2,2-dimethylpropyl)-1H-indol-5-yl)boronic acid (319 mg, 0.597 mmol) at room temperature under an atmosphere of N₂. The resulting mixture was stirred for 2 h at 70° C. under an atmosphere of N₂. The reaction mixture was extracted with DCM (3×5 mL), dried over anhydrous Na₂SO₄, filtered, and concentrated under reduced pressure to give methyl (S)-1-((3S,6R,Z)-1⁶-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-oxo-8-oxa-4-aza-1(2,4)-pyridinacycloundecaphan-10-ene-3-carbonyl)hexahydropyridazine-3-carboxylate (776 mg, crude) as a red oil. This material was taken directly to the next reaction without further purification. LCMS (ESI) m/z: [M+H] calcd for C₅₃H₇₂N₈O₇: 933.6; found 933.6.

Step 6. A mixture of methyl (S)-1-((3S,6R,Z)-1⁶-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-oxo-8-oxa-4-aza-1(2,4)-pyridinacycloundecaphan-10-ene-3-carbonyl)hexahydropyridazine-3-carboxylate (350 mg, crude) and Pd(OH)₂ (350 mg, 0.498 mmol, 20% on carbon) in MeOH (5 mL) was stirred for 1 hat room temperature under an atmosphere of H₂. The resulting mixture was filtered through Celite®, the filter cake washed with MeOH (3×10 mL), and the filtrate concentrated under reduced pressure to give methyl (S)-1-((3S,6R)-1⁶-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-oxo-8-oxa-4-aza-1(2,4)-pyridinacycloundecaphane-3-carbonyl)hexahydropyridazine-3-carboxylate (216 mg, crude) as a yellow oil. This material was taken directly to the next reaction without further purification. LCMS (ESI) m/z: [M+H] calcd for C₅₃H₇₄N₈O₇: 935.6; found 935.5.

Step 7. To a stirred solution of methyl (S)-1-((3S,6R)-1⁶-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-oxo-8-oxa-4-aza-1(2,4)-pyridinacycloundecaphane-3-carbonyl)hexahydropyridazine-3-carboxylate (200 mg, crude) in THF (1 mL) was added a solution of LiOH·H₂O (18.0 mg, 0.428 mmol) in H₂O (1 mL) dropwise at 0° C. The resulting mixture was stirred for 2 h at 0° C. The reaction mixture was then acidified to pH 4 by the addition of 1 M aq. HCl, extracted with EtOAc (3×5 mL), dried over anhydrous Na₂SO₄, filtered, and concentrated under reduced pressure to give (S)-1-((3S,6R)-1⁶-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-oxo-8-oxa-4-aza-1(2,4)-pyridinacycloundecaphane-3-carbonyl)hexahydropyridazine-3-carboxylic acid (129 mg, crude) as a brown solid. This material was taken directly to the next reaction without further purification. LCMS (ESI) m/z: [M+H] calcd for C₅₂H₇₂N₈O₇: 921.6; found 921.4.

Step 8. To a stirred solution of (S)-1-((3S,6R)-1⁶-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-oxo-8-oxa-4-aza-1(2,4)-pyridinacycloundecaphane-3-carbonyl)hexahydropyridazine-3-carboxylic acid (120 mg, crude) and DIEA (505 mg, 3.90 mmol) in DCM (15 mL) were added HOBt (176 mg, 1.30 mmol) and EDCI (499 mg, 2.60 mmol) in portions at room temperature. The resulting mixture was stirred for 2 h at 40° C. The reaction mixture was then acidified to pH 4 by the addition of 1 M aq. HCl, extracted with DCM (3×10 mL), dried over anhydrous Na₂SO₄, filtered, concentrated under reduced pressure, and purified by reversed-phase prep-HPLC to afford (9S,15S,18R)-3-(5-(4-cyclopropylpiperazin-1-yl)-2-((S)-1-methoxyethyl)pyridin-3-yl)-2-ethyl-18-isopropyl-5,5-dimethyl-2,4,5,6,9,10,11,12,15,16,18,19,22,23-tetradecahydro-8H,21H-9,13-epimino-1,29-etheno-15,26-methano-24,28-(metheno)pyrrolo[3,4-r][1,14]dioxa[5,8,23]triazacyclononacosine-8,14,17-trione (15.2 mg, 9% yield over 4 steps) as a white solid. LCMS (ESI) m/z: [M+H] calcd for C₅₂H₇₀N₈O₆: 903.5; found 903.5; ¹H NMR (400 MHz, DMSO-d₆) δ 8.78 (s, 1H), 8.45 (d, J=2.6 Hz, 1H), 8.33 (d, J=10.6 Hz, 1H), 7.91 (d, J=9.5 Hz, 1H), 7.70 (s, 1H), 7.56 (d, J=8.6 Hz, 1H), 7.31-7.17 (m, 2H), 5.70-5.60 (m, 1H), 5.20 (d, J=12.4 Hz, 1H), 4.38-4.22 (m, 2H), 4.23-3.95 (m, 4H), 3.79-3.70 (m, 1H), 3.69-3.61 (m, 1H), 3.61-3.53 (m, 1H), 3.51-3.44 (m, 1H), 3.33-3.09 (m, 7H), 3.09-3.00 (m, 1H), 3.00-2.82 (m, 3H), 2.82-2.51 (m, 8H), 2.51-2.36 (m, 1H), 2.35-2.12 (m, 2H), 2.12-1.93 (m, 2H), 1.93-1.70 (m, 3H), 1.70-1.59 (m, 2H), 1.59-1.37 (m, 3H), 1.37-1.36 (m, 2H), 1.36-0.68 (m, 12H), 0.68-0.57 (m, 1H), 0.56-0.20 (m, 6H).

### Synthesis of Compound A158—N-((9S,15S,18S,22R)-2-ethyl-18-isopropyl-3-(2-((S)-1-methoxyethyl)pyridin-3-yl)-5,5,19-trimethyl-8,14,17,20-tetraoxo-2,4,5,6,9,10,11,12,15,16,17,18,19,20,21,22,23,24-octadecahydro-8H,14H-9,13-epimino-1,31-etheno-15,28-methano-26,30-(metheno)pyrrolo[3,4-a₁][1,19]dioxa[7,10,13]triazacyclohentriacontin-22-yl)acrylamide

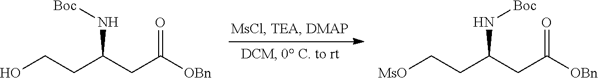 [SMILES: CC(C)(C)OC(=O)N[C@H](CCO)CC(=O)OCc1ccccc1 ; CC(C)(C)OC(=O)N[C@H](CCOS(C)(=O)=O)CC(=O)OCc1ccccc1]

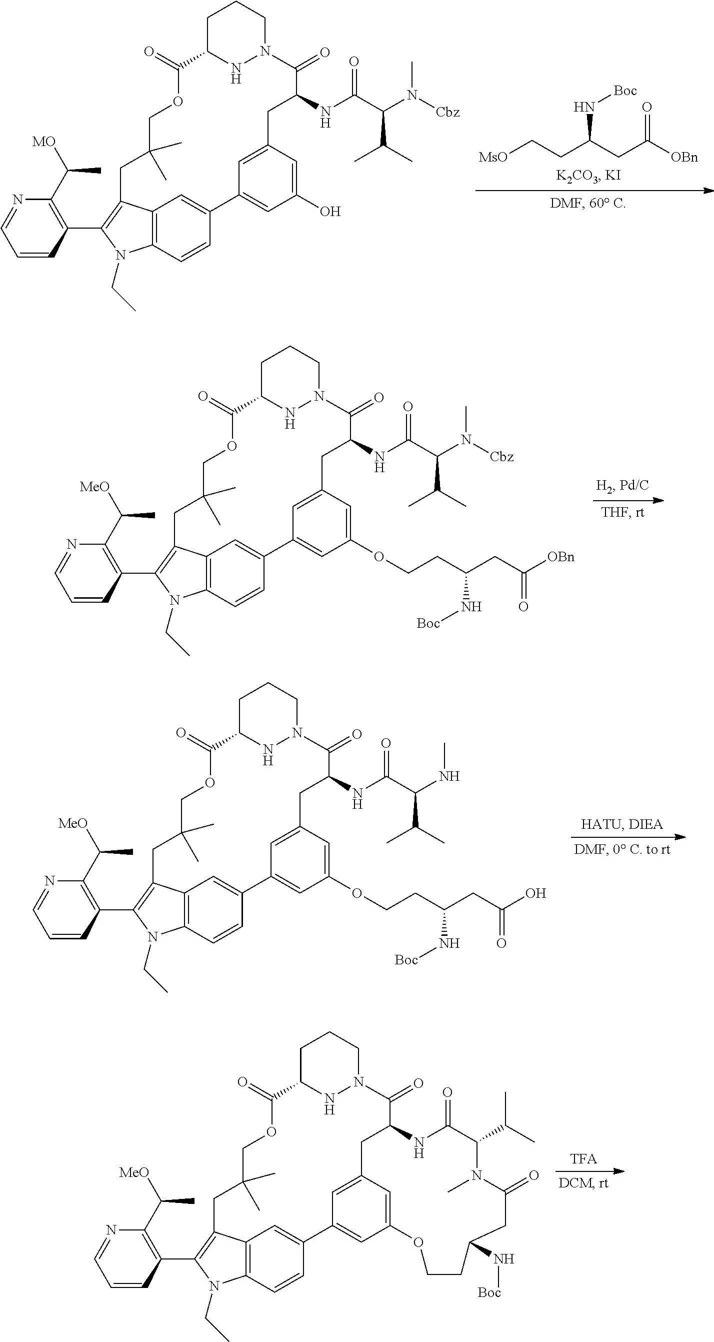

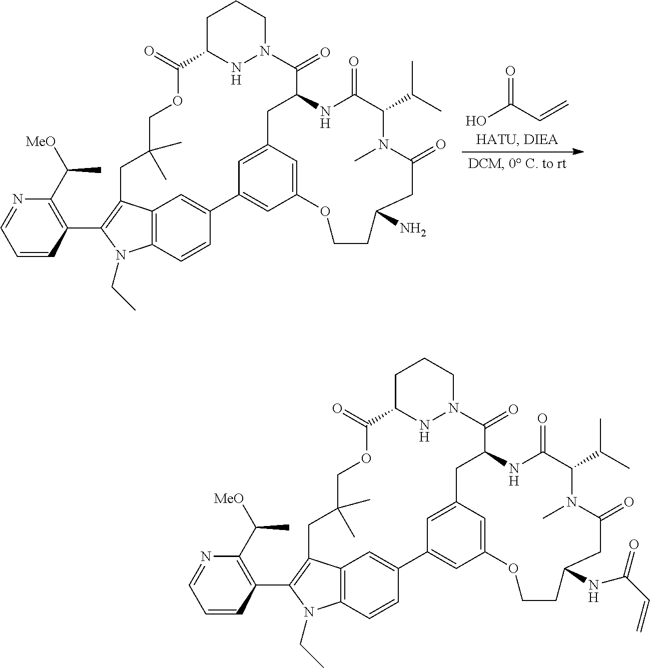

Step 1. To a mixture of benzyl (R)-3-((tert-butoxycarbonyl)amino)-5-hydroxypentanoate (3.00 g, 9.30 mmol) and TEA (1.90 g, 18.8 mmol) in DCM (30 mL) stirred at 0° C. under an atmosphere of N₂ were added DMAP (120 mg, 1.00 mmol) and MsCl (2.15 g, 18.8 mmol) in portions. The resulting mixture was stirred for 18 hours at room temperature. The reaction mixture was then diluted with H₂O (50 mL), extracted with EtOAc (3×50 mL), washed with brine (3×50 mL), dried over anhydrous Na₂SO₄, filtered, concentrated under reduced pressure, and purified by normal phase flash column chromatography to give benzyl (R)-3-((tert-butoxycarbonyl)amino)-5-((methylsulfonyl)oxy) pentanoate (1.0 g, 23% yield) as a solid. LCMS (ESI) m/z: [M+H] calcd for C₁₈H₂₇NO₇S: 402.2; found 401.8.

Step 2. To a mixture of benzyl ((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (500 mg, 0.56 mmol) and benzyl (R)-3-((tert-butoxycarbonyl)amino)-5-((methylsulfonyl)oxy)pentanoate (570 mg, 1.42 mmol) stirred in DMF (5 mL) at room temperature under an atmosphere of N₂ were added KI (30 mg, 0.30 mmol) and K₂CO₃ (200 mg, 1.50 mmol) in portions. The resulting mixture was stirred for 18 hours at 70° C. The reaction mixture was then diluted with H₂O (50 mL), extracted with EtOAc (3×50 mL), washed with brine (3×50 mL), dried over anhydrous Na₂SO₄, filtered, and concentrated under reduced pressure to give benzyl (3R)-5-(((6³S,4S)-4-((S)-2-(((benzyloxy)carbonyl)(methyl)amino)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-25-yl)oxy)-3-((tert-butoxycarbonyl)amino)pentanoate (290 mg, crude) as an oil. This material was taken directly to the next reaction without further purification. LCMS (ESI) m/z: [M+H] calcd for C₆₈H₈₅N₇O₁₂: 1192.6; found 1192.5.

Step 3. A mixture of benzyl (3R)-5-(((6³S,4S)-4-((S)-2-(((benzyloxy)carbonyl)(methyl)amino)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)oxy)-3-((tert-butoxycarbonyl)amino)pentanoate (280 mg, crude) and Pd/C (30 mg) in THF (3 mL) was stirred for 18 hours at room temperature under an atmosphere of H₂. The reaction mixture was then filtered, the filter cake was washed with EtOAc (3×50 mL) and the filtrate was concentrated under reduced pressure to give (3R)-3-((tert-butoxycarbonyl)amino)-5-(((6³S,4S)-1¹-ethyl-1²-(2-((S)-1-methoxyethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)oxy)pentanoic acid (300 mg, crude) as a solid. This material was taken directly to the next reaction without further purification. LCMS (ESI) m/z: [M+H] calcd for C₅₃H₇₃N₇O₁₀: 968.5; found 968.6.

Step 4. To a mixture (3R)-3-((tert-butoxycarbonyl)amino)-5-(((6³S,4S)-1¹-ethyl-1²-(2-((S)-1-methoxyethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl)oxy)pentanoic acid (300 mg, crude) in DMF (2 mL) stirred at 0° C. under an atmosphere of N₂ were added DIPEA (400 mg, 3.10 mmol) and HATU (415 mg, 1.10 mmol). The resulting mixture was stirred for 2 hours at room temperature. The reaction mixture was then diluted with H₂O (50 mL), extracted with EtOAc (3×50 mL), washed with brine (3×50 mL), dried over anhydrous Na₂SO₄, filtered, concentrated under reduced pressure, and purified by normal phase flash column chromatography to give tert-butyl ((9S,15S,18S,22R)-2-ethyl-18-isopropyl-3-(2-((S)-1-methoxyethyl)pyridin-3-yl)-5,5,19-trimethyl-8,14,17,20-tetraoxo-2,4,5,6,9,10,11,12,15,16,17,18,19,20,21,22,23,24-octadecahydro-8H,14H-9,13-epimino-1,31-etheno-15,28-methano-26,30-(metheno)pyrrolo[3,4-a₁][1,19]dioxa[7,10,13]triazacyclohentriacontin-22-yl)carbamate (140 mg, 27% yield over 3 steps) as a solid. LCMS (ESI) m/z: [M+H] calcd for C₅₃H₇₁N₇O₉: 950.5; found 950.6.

Step 5. To a mixture of tert-butyl ((9S,15S,18S,22R)-2-ethyl-18-isopropyl-3-(2-((S)-1-methoxyethyl)pyridin-3-yl)-5,5,19-trimethyl-8,14,17,20-tetraoxo-2,4,5,6,9,10,11,12,15,16,17,18,19,20,21,22,23,24-octadecahydro-8H,14H-9,13-epimino-1,31-etheno-15,28-methano-26,30-(metheno)pyrrolo[3,4-a₁][1,19]dioxa[7,10,13]triazacyclohentriacontin-22-yl)carbamate (130 mg, 0.12 mmol) in DCM (2 mL) stirred at 0° C. under an atmosphere of N₂ was added TFA (0.5 mL). The reaction mixture was stirred for 2 hours at room temperature and was then concentrated under reduced pressure to give (9S,15S,18S,22R)-22-amino-2-ethyl-18-isopropyl-3-(2-((S)-1-methoxyethyl)pyridin-3-yl)-5,5,19-trimethyl-2,4,5,6,9,10,11,12,15,16,18,19,21,22,23,24-hexadecahydro-8H,14H-9,13-epimino-1,31-etheno-15,28-methano-26,30-(metheno)pyrrolo[3,4-a₁][1,19]dioxa[7,10,13]triazacyclohentriacontine-8,14,17,20-tetraone (200 mg, crude) as an oil. This material was taken directly to the next reaction without further purification. LCMS (ESI) m/z: [M+H] calcd for C₄₈H₆₃N₇O₇: 950.5; found 950.6.

Step 6. To a stirred mixture of (9S,15S,18S,22R)-22-amino-2-ethyl-18-isopropyl-3-(2-((S)-1-methoxyethyl)pyridin-3-yl)-5,5,19-trimethyl-2,4,5,6,9,10,11,12,15,16,18,19,21,22,23,24-hexadecahydro-8H,14H-9,13-epimino-1,31-etheno-15,28-methano-26,30-(metheno)pyrrolo[3,4-a₁][1,19]dioxa[7,10,13]triazacyclohentriacontine-8,14,17,20-tetraone (100 mg, crude) in DCM (5 mL) at 0° C. were added DIPEA (152 mg, 1.18 mmol), acrylic acid (20 mg, 0.28 mmol) and HATU (60 mg, 0.16 mmol). The resulting mixture was stirred at room temperature for 2 hours. The reaction mixture then concentrated under concentrated under reduced pressure, diluted with H₂O (20 mL), extracted with EtOAc (3×20 mL), washed with H₂O (3×10 mL), dried over anhydrous Na₂SO₄, filtered, concentrated under reduced pressure, and purified by reversed-phase prep-HPLC to give N-((9S,15S,18S,22R)-2-ethyl-18-isopropyl-3-(2-((S)-1-methoxyethyl)pyridin-3-yl)-5,5,19-trimethyl-8,14,17,20-tetraoxo-2,4,5,6,9,10,11,12,15,16,17,18,19,20,21,22,23,24-octadecahydro-8H,14H-9,13-epimino-1,31-etheno-15,28-methano-26,30-(metheno)pyrrolo[3,4-a₁][1,19]dioxa[7,10,13]triazacyclohentriacontin-22-yl)acrylamide (22 mg, 41% yield over 2 steps) as a solid. LCMS (ESI) m/z: [M+H] calcd for C₅₁H₆₆N₇O₈: 950.5; found 950.6; ¹H NMR (300 MHz, DMSO-d₆) δ 8.81-8.68 (m, 2H), 8.26 (d, J=8.2 Hz, 1H), 7.99 (s, 1H), 7.79 (dd, J=7.7, 1.8 Hz, 1H), 7.68 (d, J=8.8 Hz, 1H), 7.64-7.49 (m, 2H), 7.32 (s, 1H), 7.18 (s, 1H), 6.81 (s, 1H), 6.31-6.09 (m, 2H), 5.63 (dd, J=9.7, 2.6 Hz, 1H), 5.18 (t, J=8.9 Hz, 1H), 5.03 (d, J=12.3 Hz, 1H), 4.94 (d, J=11.2 Hz, 1H), 4.50 (s, 1H), 4.36-4.23 (m, 4H), 4.21-4.04 (m, 2H), 3.97-3.87 (m, 1H), 3.80-3.57 (m, 3H), 3.25 (s, 3H), 3.04 (d, J=14.4 Hz, 2H), 2.95 (s, 3H), 2.72 (q, J=13.2 Hz, 3H), 2.46-2.32 (m, 2H), 2.15-1.67 (m, 7H), 1.56 (d, J=11.1 Hz, 1H), 1.37 (d, J=6.1 Hz, 3H), 1.01-0.72 (m, 13H), 0.36 (s, 3H).

### Synthesis of Compound A164—(9S,15S,18S)-22-acryloyl-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,16,19]pentaazacyclohentriacontine-8,14,17,20(21H)-tetraone

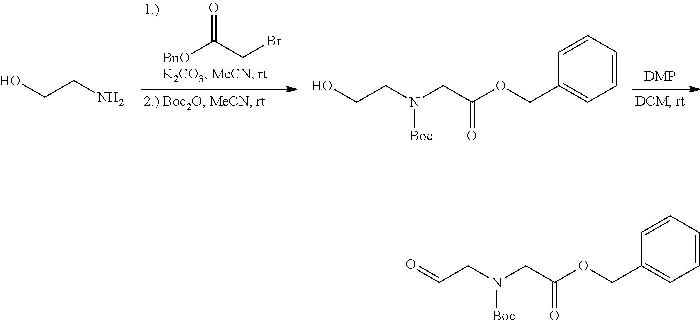

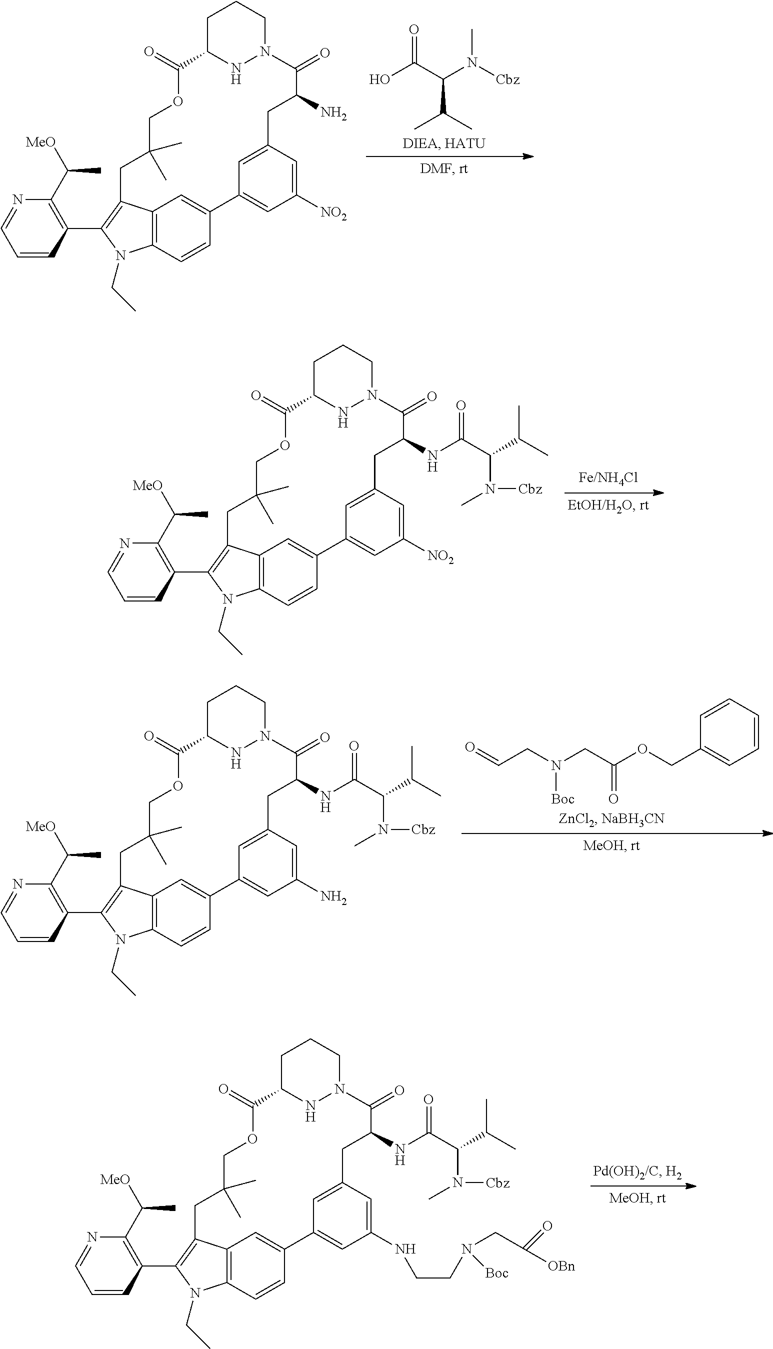

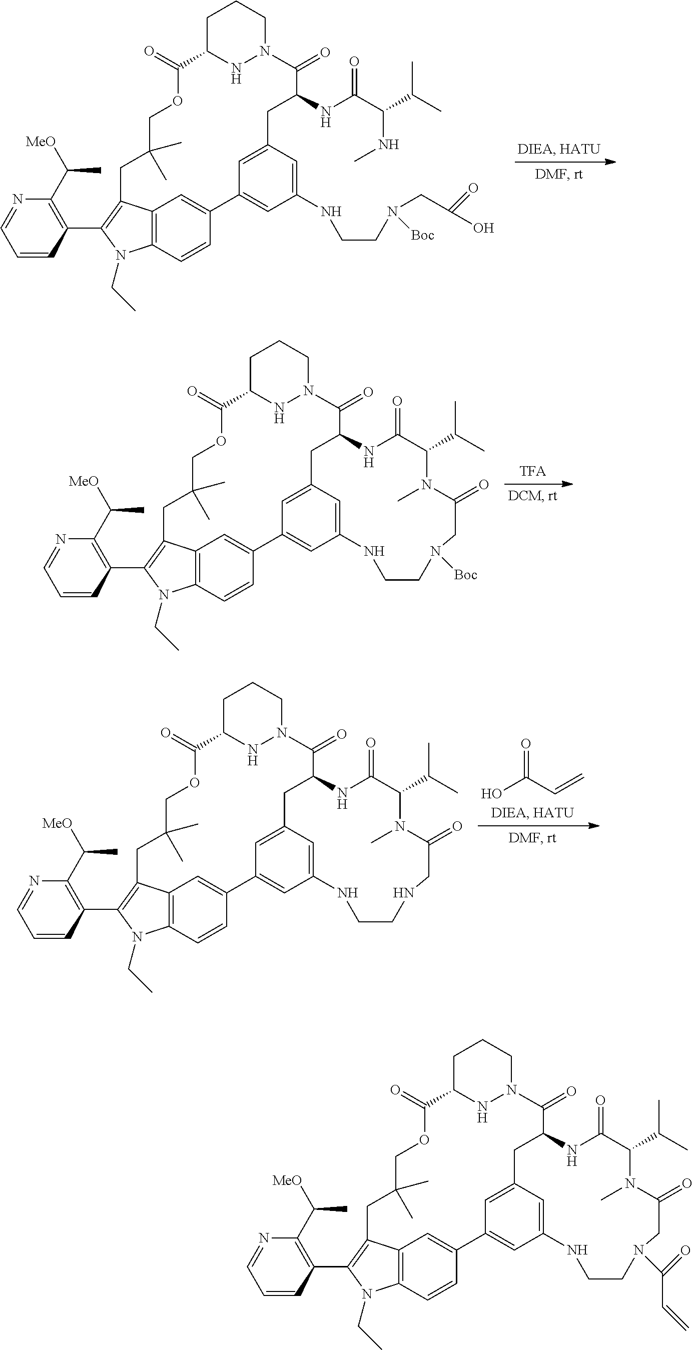

Step 1. To a stirred solution of 2-aminoethan-1-ol (990 mg, 16.2 mmol) and K₂CO₃ (9.35 g, 67.7 mmol) in MeCN (10 mL) was added benzyl 2-bromoacetate (3.10 g, 13.5 mmol) dropwise at 0° C. The resulting mixture was stirred for 45 minutes at room temperature. Boc₂O (8.86 g, 40.6 mmol) was subsequently added and the resulting mixture was stirred for 1 hour at room temperature. The reaction mixture was treated with brine (30 mL), extracted with DCM (3×30 mL), concentrated under reduced pressure, and purified by normal phase flash column chromatography to afford benzyl N-(tert-butoxycarbonyl)-N-(2-hydroxyethyl)glycinate (1.36 g, 26% yield, 80% purity) as a light yellow oil. LCMS (ESI) m/z: [M+Na] calcd for C₁₉H₂₈O₅: 332.2; found 332.2.

Step 2. To a stirred solution of benzyl N-(tert-butoxycarbonyl)-N-(2-hydroxyethyl)glycinate (500 mg, 1.62 mmol) in DCM (5 mL) was added DMP (1.37 g, 3.23 mmol) in portions at 0° C. The resulting mixture was stirred for 2 hours at room temperature. The reaction mixture was then quenched by the addition of sat. aq. NaHCO₃ at 0° C., extracted with DCM (3×20 mL), and concentrated under reduced pressure to afford benzyl N-(tert-butoxycarbonyl)-N-(2-oxoethyl)glycinate (650 mg, crude) as a light yellow oil. This material was taken directly to the next reaction without further purification. LCMS (ESI) m/z: [M−C₄H₈+H] calcd for C₁₆H₂₁NO₅: 252.1; found 252.2.

Step 3. To a stirred solution of (6³S,4S)-4-amino-1¹-ethyl-1²-(2-((S)-1-methoxyethyl)pyridin-3-yl)-10,10-dimethyl-2⁵-nitro-6¹,6²,6³,6⁴,6⁵,6⁶-hexahydro-1¹H-8-oxa-1(5,3)-indola-6(1,3)-pyridazina-2(1,3)- benzenacycloundecaphane-5,7-dione (905 mg, 1.35 mmol) and N-((benzyloxy)carbonyl)-N-methyl-L-valine (718 mg, 2.71 mmol) in DMF (30 mL) were added DIEA (874 mg, 6.77 mmol) and HATU (926 mg, 2.44 mmol) in portions at room temperature. The resulting mixture was stirred for 2 hours at room temperature. The reaction mixture was then diluted with H₂O (100 mL), extracted with EtOAc (3×50 mL), washed with H₂O (3×100 mL), dried over anhydrous Na₂SO₄, filtered, concentrated under reduced pressure, and purified by reversed-phase flash column chromatography to afford benzyl ((2S)-1-(((6³S,4S)-1¹-ethyl-1²-(2-((S)-1-methoxyethyl)pyridin-3-yl)-10,10-dimethyl-2⁵-nitro-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (940 mg, 72% yield) as a yellow oil. LCMS (ESI) m/z: [M+H] calcd for C₅₁H₆₁N₇O₉: 916.5; found 916.5.

Step 4. To a stirred mixture of benzyl ((2S)-1-(((6³S,4S)-1¹-ethyl-1²-(2-((S)-1-methoxyethyl)pyridin-3-yl)-10,10-dimethyl-2⁵-nitro-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (600 mg, 0.66 mmol) in EtOH (10 mL) and H₂O (10 mL) were added NH₄Cl (175 mg, 3.3 mmol) and Fe powder (183 mg, 3.3 mmol) in portions. The resulting mixture was stirred at room temperature for 3 hours. The reaction mixture was then filtered, the filter cake was washed with MeOH (3×20 mL), the filtrate was concentrated under reduced pressure, and the residue was purified by normal phase flash column chromatography to afford benzyl ((2S)-1-(((6³S,4S)-2⁵-amino-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (340 mg, 53% yield) as a clear oil. LCMS (ESI) m/z: [M+H] calcd for C₅₁H₆₃N₇O₇: 886.5; found 886.3.

Step 5. To a stirred mixture of benzyl ((2S)-1-(((6³S,4S)-2⁵-amino-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (200 mg, 0.230 mmol) and benzyl N-(tert-butoxycarbonyl)-N-(2-oxoethyl)glycinate (208 mg, 0.68 mmol) in MeOH (15 mL) were added NaBH₃CN (71.0 mg, 1.13 mmol) and ZnCl₂ (170 mg, 1.3 mmol) in portions at room temperature. The resulting mixture was stirred at room temperature for 3 hours. The reaction mixture was then diluted with H₂O (20 mL) concentrated under reduced pressure, extracted with EtOAc (3×20 mL), concentrated under reduced pressure, and purified by reversed-phase prep-HPLC to afford benzyl N-(2-(((6³S,4S)-4-((S)-2-(((benzyloxy)carbonyl)(methyl)amino)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethyl)-N-(tert-butoxycarbonyl)glycinate (290 mg, 98% yield) as a a clear oil. LCMS (ESI) m/z: [M+H] calcd for C₆₇H₈₄N₈O₁₁: 1177.6; found 1177.4.

Step 6. A mixture of benzyl N-(2-(((6³S,4S)-4-((S)-2-(((benzyloxy)carbonyl)(methyl)amino)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethyl)-N-(tert-butoxycarbonyl)glycinate (290 mg, 0.25 mmol) and Pd(OH)₂/C (296 mg, 20% wt.) in MeOH (15 mL) was stirred at room temperature for 1 hour under an atmosphere of H₂. The reaction mixture was then filtered, the filter cake was washed with MeOH (5×20 mL), and the filtrate was concentrated under reduced pressure to give N-(tert-butoxycarbonyl)-N-(2-(((6³S,4S)-1¹-ethyl-1²-(2-((S)-1-methoxyethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethyl)glycine (105 mg, crude) as a clear oil oil. This material was taken directly to the next reaction without further purification. LCMS (ESI) m/z: [M+H] calcd for C₅₂H₇₂N₈O₉: 953.5; found 953.6.

Step 7. To a stirred mixture of N-(tert-butoxycarbonyl)-N-(2-(((6³S,4S)-1¹-ethyl-1²-(2-((S)-1-methoxyethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)ethyl)glycine (100 mg, crude) in DMF (5 mL) were added DIPEA (68.0 mg, 0.530 mmol) and HATU (80.0 mg, 0.210 mmol) in portions at room temperature. The resulting mixture was stirred at room temperature for 1 hour. The reaction mixture was diluted with H₂O, extracted with EtOAc, washed with brine, concentrated under reduced pressure, and purified by reversed-phase flash column chromatography to give tert-butyl (9S,15S,18S)-2-ethyl-18-isopropyl-3-(2-((S)-1-methoxyethyl)pyridin-3-yl)-5,5,19-trimethyl-8,14,17,20-tetraoxo-2,4,5,6,9,10,11,12,14,15,16,17,18,19,20,21,24,25-octadecahydro-8H-9,13-epimino-1,31-etheno-15,28-methano-26,30-(metheno)pyrrolo[3,4-a₁][1]oxa[7,10,13,16,19]pentaazacyclohentriacontine-22(23H)-carboxylate (26.0 mg, 12% yield over two steps) as an off-white solid. LCMS (ESI) m/z: [M+H] calcd for C₅₂H₇₀N₈O₈: 935.5; found 935.2.

Step 8. A mixture of tert-butyl (9S,15S,18S)-2-ethyl-18-isopropyl-3-(2-((S)-1-methoxyethyl)pyridin-3-yl)-5,5,19-trimethyl-8,14,17,20-tetraoxo-2,4,5,6,9,10,11,12,14,15,16,17,18,19,20,21,24,25-octadecahydro-8H-9,13-epimino-1,31-etheno-15,28-methano-26,30-(metheno)pyrrolo[3,4-a₁][1]oxa[7,10,13,16,19]pentaazacyclohentriacontine-22(23H)-carboxylate (21.0 mg, 0.020 mmol) in DCM (10 mL) and TFA (5 mL) was stirred at room temperature for 1 h. The reaction mixture was then concentrated under reduced pressure, adjusted to pH 8 by the addition of sat. aq. NaHCO₃, extracted with EtOAc (2×30 mL), and concentrated under reduced pressure to give (9S,15S,18S)-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,16,19]pentaazacyclohentriacontine-8,14,17,20(21H)-tetraone (14.0 mg, crude) as a solid. This material was taken directly to the next reaction without further purification. LCMS (ESI) m/z: [M+H] calcd for C₄₇H₆₂N₈O₆: 835.5; found 835.2.

Step 9. To a stirred mixture of (9S,15S,18S)-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,16,19]pentaazacyclohentriacontine-8,14,17,20(21H)-tetraone (44.0 mg, crude) and acrylic acid (11.0 mg, 0.16 mmol) in DMF were added DIPEA (34.0 mg, 0.270 mmol) and HATU (30.1 mg, 0.080 mmol) in portions at room temperature. The reaction mixture was stirred at room temperature for 2 hours, after which time it was concentrated under reduced pressure and was purified by reversed-phase prep-HPLC to give (9S,15S,18S)-22-acryloyl-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,16,19]pentaazacyclohentriacontine-8,14,17,20(21H)-tetraone (5.5 mg, 13% yield over two steps) as a white solid. LCMS (ESI) m/z: [M+H] calcd for C₅₀H₆₄N₈O₇: 888.5; found 888.3; ¹H NMR (400 MHz, CD₃OD) δ 8.77-8.68 (m, 1H), 8.10 (s, 1H), 7.86 (s, 1H), 7.69 (s, 1H), 7.52 (s, 2H), 7.35-7.14 (m, 1H), 7.02 (s, 1H), 6.88 (s, 1H), 6.56-6.42 (m, 1H), 6.38-6.19 (m, 1H), 5.90-5.65 (m, 1H), 5.51-5.27 (m, 1H), 5.09-4.93 (m, 1H), 4.80-4.63 (m, 1H), 4.46 (s, 1H), 4.35 (s, 1H), 4.31-4.15 (m, 2H), 4.14-3.92 (m, 3H), 3.91-3.76 (m, 3H), 3.74 (s, 1H), 3.69-3.36 (m, 3H), 3.27 (s, 1H), 3.21 (s, 1H), 3.09-2.99 (m, 1H), 2.94 (s, 2H), 2.79 (s, 3H), 2.70 (s, 1H), 2.64 (s, 1H), 2.37-2.08 (m, 2H), 2.05-1.74 (m, 2H), 1.74-1.57 (m, 1H), 1.46 (s, 3H), 1.30 (s, 1H), 1.14 (s, 2H), 1.08-0.92 (m, 6H), 0.92-0.77 (m, 4H), 0.72-0.47 (m, 3H).

### Synthesis of Compound A167—(9S,15S,18S)-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,19]tetraazacyclohentriacontine-8,14,17,20(21H)-tetraone

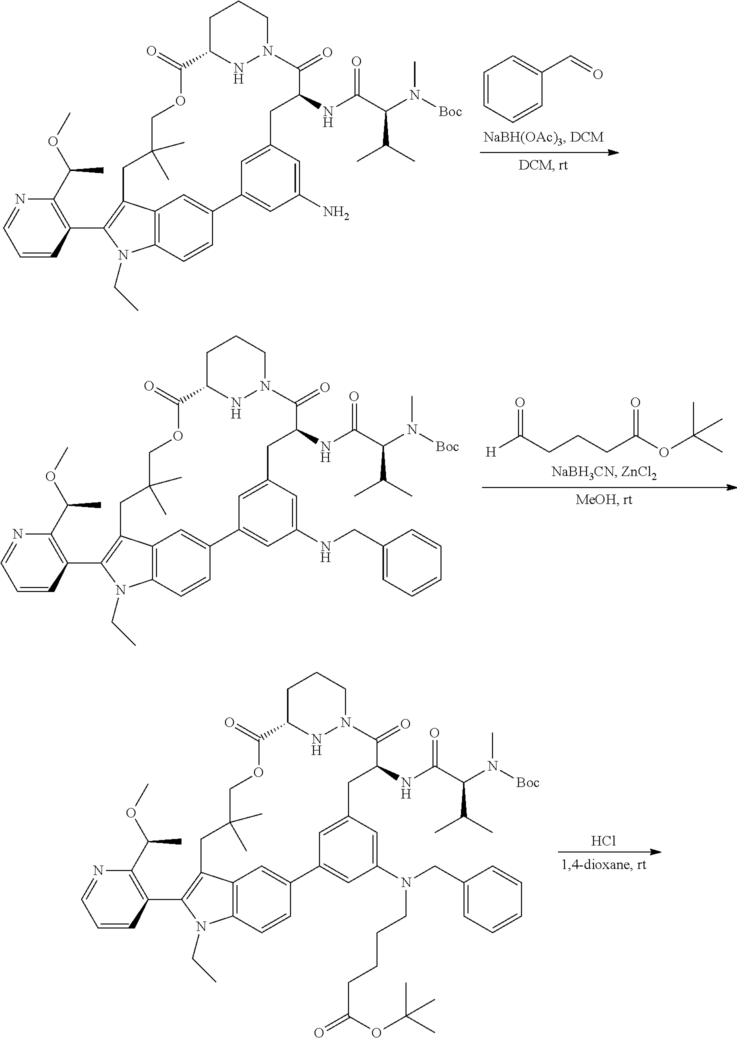

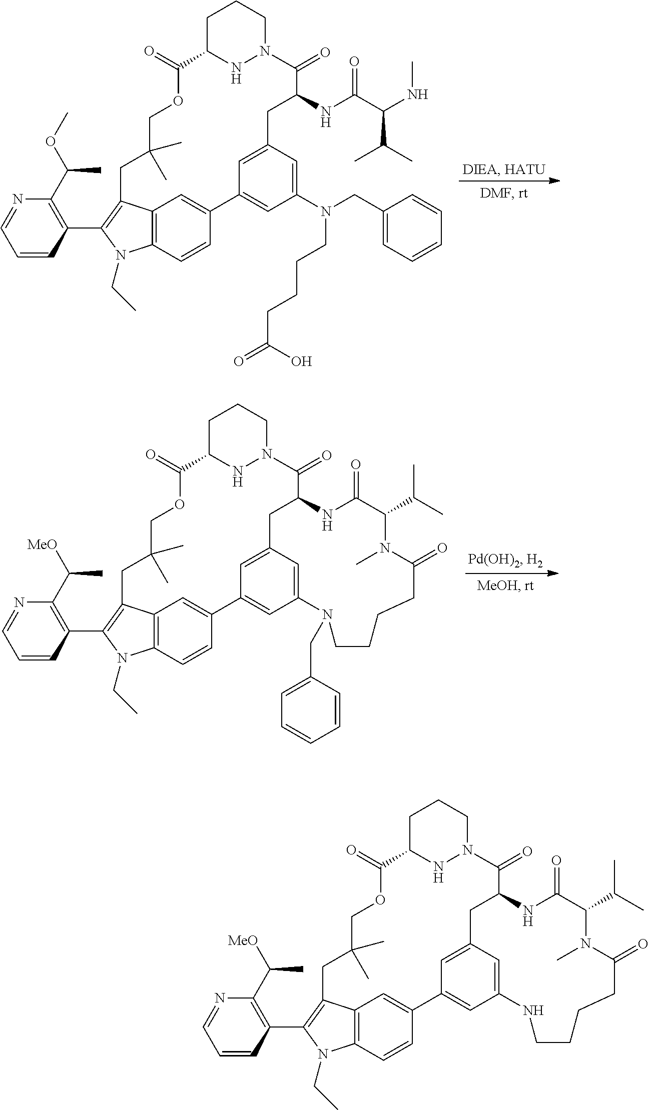

Step 1. To a stirred mixture of tert-butyl ((2S)-1-(((6³S,4S)-2⁵-amino-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (240 mg, 0.282 mmol) and benzaldehyde (120 mg, 1.13 mmol) in DCM (20 mL) was added NaBH(OAc)₃ (597 mg, 2.82 mmol) at room temperature. The resulting mixture was stirred for 2 hours at room temperature. The reaction mixture was then diluted with DCM (50 mL), washed with H₂O (3×50 mL), dried over anhydrous Na₂SO₄, filtered, concentrated under reduced pressure, and purified by normal phase prep-TLC to afford tert-butyl ((2S)-1-(((6³S,4S)-2⁵-(benzylamino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (240 mg, 72% yield) as a yellow oil. LCMS (ESI) m/z: [M+H] calcd for C₅₅H₇₁N₇O₇ 942.5; found 942.5.

Step 2. To a stirred mixture of tert-butyl ((2S)-1-(((6³S,4S)-2⁵-(benzylamino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (125 mg, 0.133 mmol) and tert-butyl 5-oxopentanoate (114 mg, 0.663 mmol) in MeOH (20 mL) were added ZnCl₂ (181 mg, 1.33 mmol) and NaBH₃CN (83.4 mg, 1.33 mmol) at room temperature. The resulting mixture was stirred for 2 hours at room temperature. The reaction mixture was then concentrated under reduced pressure, diluted in H₂O (100 mL), extracted with EtOAc (2×100 mL), washed with H₂O (3×100 mL), dried over anhydrous Na₂SO₄, filtered, concentrated under reduced pressure, and purified by normal phase flash column chromatography to afford tert-butyl 5-(benzyl((6³S,4S)-4-((S)-2-((tert-butoxycarbonyl)(methyl)amino)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)pentanoate (100 mg, 48% yield) as a yellow oil. LCMS (ESI) m/z: [M+H] calcd for C₆₄H₈₇N₇O₉ 1098.7; found 1098.5.

Step 3. tert-butyl 5-(benzyl((6³S,4S)-4-((S)-2-((tert-butoxycarbonyl)(methyl)amino)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)pentanoate (100 mg, 0.091 mmol) was added to a stirred solution of HCl in 1,4-dioxane (4.0 M, 10.0 mL) at room temperature. The resulting mixture was stirred for 2 hours at room temperature and was then concentrated under reduced pressure to afford tert-butyl 5-(benzyl((6³S,4S)-1¹-ethyl-1²-(2-((S)-1-methoxyethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)pentanoate (100 mg, crude) as a white solid. This material was taken directly to the next reaction without further purification.

Step 4. To a stirred mixture of tert-butyl 5-(benzyl((6³S,4S)-1¹-ethyl-1²-(2-((S)-1-methoxyethyl)pyridin-3-yl)-10,10-dimethyl-4-((S)-3-methyl-2-(methylamino)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)pentanoate (100 mg, crude) and DIEA (137 mg, 1.06 mmol) in DMF (8.0 mL) was added HATU (121 mg, 0.318 mmol) at room temperature. The resulting mixture was stirred for 4 hours at room temperature. The reaction mixture was then concentrated under reduced pressure and purified by reversed-phase flash column chromatography to afford (9S,15S,18S)-25-benzyl-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,19]tetraazacyclohentriacontine-8,14,17,20(21H)-tetraone (80.0 mg, 95% yield over 2 steps) as a white solid. LCMS (ESI) m/z: [M/2+H] calcd for C₅₅H₆₉N₇O₆ 462.8; found 463.0.

Step 5. A mixture of (9S,15S,18S)-25-benzyl-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,19]tetraazacyclohentriacontine-8,14,17,20(21H)-tetraone (80.0 mg, 0.087 mmol) and Pd(OH)₂ (60.0 mg, 20% wt.) in MeOH (5.0 mL) was stirred for 4 hours at room temperature under an atmosphere of H₂. The reaction mixture was then concentrated under reduced pressure and purified by reversed-phase flash column chromatography to afford (9S,15S,18S)-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,31-etheno-15,28-methano-26,30-(metheno)pyrrolo[3,4-a₁][1]oxa[7,10,13,19]tetraazacyclohentriacontine-8,14,17,20(21H)-tetraone (3.6 mg, 4.9% yield) as a white solid. LCMS (ESI) m/z: [M+H] calcd for C₄₈H₆₃N₇O₆: 834.5; found 835.5. ¹H NMR (400 MHz, DMSO-d₆) δ 8.78-8.67 (m, 1H), 8.12-8.02 (m, 1H), 7.92-7.82 (m, 1H), 7.72-7.62 (m, 1H), 7.59-7.53 (m, 1H), 7.53-7.45 (s, 1H), 7.22-7.06 (m, 1H), 6.97 (s, 1H), 6.45-6.32 (m, 1H), 5.55-5.40 (m, 1H), 5.12-5.08 (m, 2H), 4.49-4.03 (m, 5H), 4.00-3.50 (m, 5H), 3.25 (s, 3H), 3.19-3.01 (m, 2H), 2.98 -2.60 (m, 9H), 2.40-2.10 (m, 3H), 2.01-1.91 (m, 1H), 1.91-1.65 (m, 6H), 1.50-1.40 (m, 4H), 1.37-1.17 (m, 1H), 1.15-1.05 (m, 1H), 1.06-0.91 (m, 6H), 0.91-0.85 (s, 4H), 0.84-0.80 (m, 3H), 0.57 (s, 3H).

### Synthesis of Compound A179—(9S,15S,18S,Z)-2-ethyl-18-isopropyl-3-(2-((S)-1-methoxyethyl)pyridin-3-yl)-5,5,19-trimethyl-2,4,5,6,9,10,11,12,15,16,18,19,23,24-tetradecahydro-8H,14H-9,13-epimino-1,30-etheno-15,27-methano-25,29-(metheno)pyrrolo[3,4-z][1]oxa[7,10,13,18]tetraazacyclotriacontine-8,14,17,20-tetraone

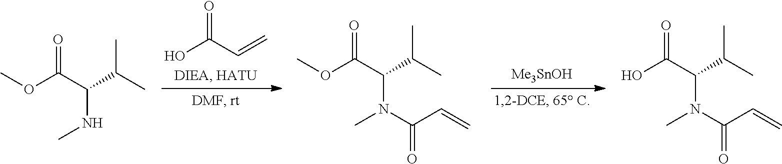

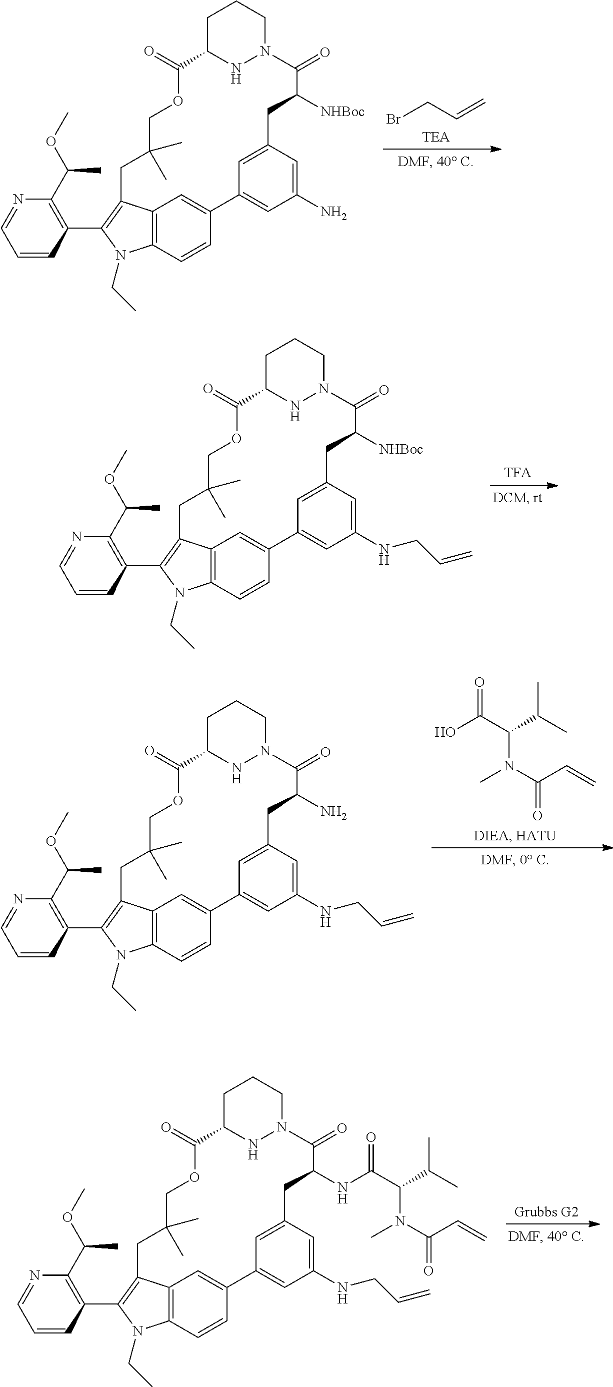

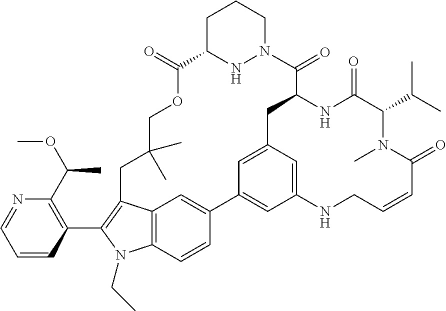

Step 1. To a stirred solution of methyl-L-valinate (200 mg, 1.40 mmol) in DMF (2 mL) were added prop-2-enoic acid (149 mg, 2.10 mmol) and DIPEA (1.78 g, 13.8 mmol) at room temperature. The resulting mixture was stirred for 10 minutes at room temperature, after which HATU (630 mg, 1.10 mmol) was added and the mixture stirred at room temperature for 30 minutes. The reaction mixture was then diluted with EtOAc (200 mL), washed with H₂O (4×100 mL), treated with brine (100 mL), dried over anhydrous Na₂SO₄, filtered, concentrated under reduced pressure, and purified by normal phase flash column chromatography to afford methyl N-acryloyl-N-methyl-L-valinate (200 mg, 65% yield) as a clear oil. LCMS (ESI) m/z: [M+H] calcd for C₁₀H₁₇NO₃: 200.1; found 200.1.

Step 2. A stirred mixture of methyl N-acryloyl-N-methyl-L-valinate (200 mg, 0.80 mmol) and Me₃SnOH (1.46 g, 8.10 mmol) in 1,2-DCE (30 mL) was heated to 65° C. and stirred for 8 hours. The reaction mixture was then diluted with H₂O (20 mL), extracted with DCM (3×100 mL), and concentrated under reduced pressure to give N-acryloyl-N-methyl-L-valine (120 mg, crude) as an oil. This material was taken directly to the next reaction without further purification. LCMS (ESI) m/z: [M+H] calcd for C₉H₁₅NO₃: 186.1; found 186.1.

Step 3. To a stirred mixture of tert-butyl ((6³S,4S)-2⁵-amino-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (400 mg, 0.50 mmol) in DMF (12 mL) was added 3-bromoprop-1-ene (261 mg, 2.20 mmol) and TEA (352 mg, 1.1 mmol) at room temperature. The resulting mixture was heated to 40° C. and stirred for 2 hours. The reaction mixture was then diluted with EtOAc (100 mL), washed with H₂O (4×100 mL), treated with brine (100 mL), dried over anhydrous Na₂SO₄, filtered, concentrated under reduced pressure, and purified by normal phase prep-TLC to afford tert-butyl ((6³S,4S)-25-(allylamino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (180 mg, 36% yield) as a yellow solid. LCMS (ESI) m/z: [M+H] calcd for C₄₅H₅₈N₆O₆: 779.4; found 779.4.

Step 4. To a stirred mixture of tert-butyl ((6³S,4S)-25-(allylamino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (270 mg, crude) in DCM (3.0 mL) was added TFA (1.0 mL). The resulting mixture was stirred at room temperature for 1 hour and was then concentrated under reduced pressure to give (6³S,4S)-2⁵-(allylamino)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (270 mg, crude) as a yellow solid. This material was taken directly to the next reaction without further purification. LCMS (ESI) m/z: [M+H] calcd for C₄₀H₅₀N₆O₄: 679.4; found 679.4.

Step 5. To a mixture of (6³S,4S)-2⁵-(allylamino)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (230 mg, crude) and (2S)-3-methyl-2-(N-methylprop-2-enamido)butanoic acid (82.0 mg, crude) in DMF (5 mL) stirred at 0° C. was added DIEA (439 mg, 3.40 mmol) and a solution of HATU (155 mg, 0.41 mmol) in DMF (5 mL). The resulting mixture was stirred at 0° C. for 1 hour. The reaction mixture was diluted with EtOAc (80 mL) and cold H₂O (40 mL) at 0° C. and the organic extract was washed with H₂O (3×40 mL), treated with brine (40 mL), dried over anhydrous Na₂SO₄, filtered, concentrated under reduced pressure, and purified by normal phase prep-TLC to afford (2S)—N-((6³S,4S)-2⁵-(allylamino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(N-methylacrylamido)butanamide (70 mg, 19% yield over 2 steps) as a white solid. LCMS (ESI) m/z: [M+H] calcd for C₄₉H₆₃N₇O₆: 846.5; found 846.5.

Step 6. To a mixture of (2S)—N-((6³S,4S)-2⁵-(allylamino)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(N-methylacrylamido)butanamide (60.0 mg, 0.10 mmol) stirred in DCM (10 mL) under an atmosphere of N₂ was added Grubbs 2^{nd} generation catalyst (30.0 mg, 0.04 mmol). The resulting mixture was stirred at 40° C. for 16 hours. The reaction mixture was then diluted with DCM (10 mL), washed with H₂O (4×10 mL), treated with brine (10 mL), dried over anhydrous Na₂SO₄, filtered, concentrated under reduced pressure, and was purified by normal phase prep-TLC to afford (9S,15S,18S,Z)-2-ethyl-18-isopropyl-3-(2-((S)-1-methoxyethyl)pyridin-3-yl)-5,5,19-trimethyl-2,4,5,6,9,10,11,12,15,16,18,19,23,24-tetradecahydro-8H,14H-9,13-epimino-1,30-etheno-15,27-methano-25,29-(metheno)pyrrolo[3,4-z][1]oxa[7,10,13,18]tetraazacyclotriacontine-8,14,17,20-tetraone (10.5 mg, 16% yield) as a white solid. LCMS (ESI) m/z: [M+H] calcd for C₄₇H₅₉N₇O₆: 818.5; found 818.5; ¹H NMR (400 MHz, CD₃OD) δ 8.73 (dd, J=4.7, 1.7 Hz, 1H), 8.55 (s, 1H), 8.04 (m, 1H), 7.90-7.78 (m, 1H), 7.74-7.63 (m, 1H), 7.58-7.40 (m, 2H), 7.17 (d, J=17.6 Hz, 1H), 7.19-6.82 (m, 1H), 6.59-6.42 (m, 1H), 6.37-6.25 (m, 1H), 6.05 (m, 1H), 5.42-5.22 (m, 2H), 4.56-4.37 (m, 1H), 4.36-4.19 (m, 2H), 4.18-3.92 (m, 2H), 3.89-3.69 (m, 2H), 3.67-3.40 (m, 2H), 3.37 (d, J=4.6 Hz, 1H), 3.29-3.20 (m, 1H), 3.19-3.04 (m, 2H), 3.04-2.85 (m, 2H), 2.80-2.71 (m, 2H), 2.28-2.12 (m, 2H), 2.07-1.79 (m, 3H), 1.77-1.56 (m, 2H), 1.45 (m, 3H), 1.28 (d, J=8.1 Hz, 3H), 1.20 (d, J=6.6 Hz, 1H), 1.04-0.83 (m, 10H), 0.61-0.42 (m, 2H).

### Synthesis of Compound A142—(2²Z,2³aZ,2⁵S,2⁸S,4³S)-1¹-ethyl-2⁸-isopropyl-1²-(2-((S)-1-methoxyethyl)pyridin-3-yl)-2⁹,8,8-trimethyl-2⁴,2⁵,2⁶,2⁷2⁸,2⁹,2¹⁰,2¹¹2¹²,2¹³4¹,4²,4³,4⁴,4⁵,4⁶-hexadecahydro-1¹H-6-oxa-2(2,5)-thiazolo[4,5-g][1,4]diazacyclododecina-1(5,3)-indola-4(1,3)-pyridazinacyclononaphane-2⁷ 2¹⁰,3,5-tetraone

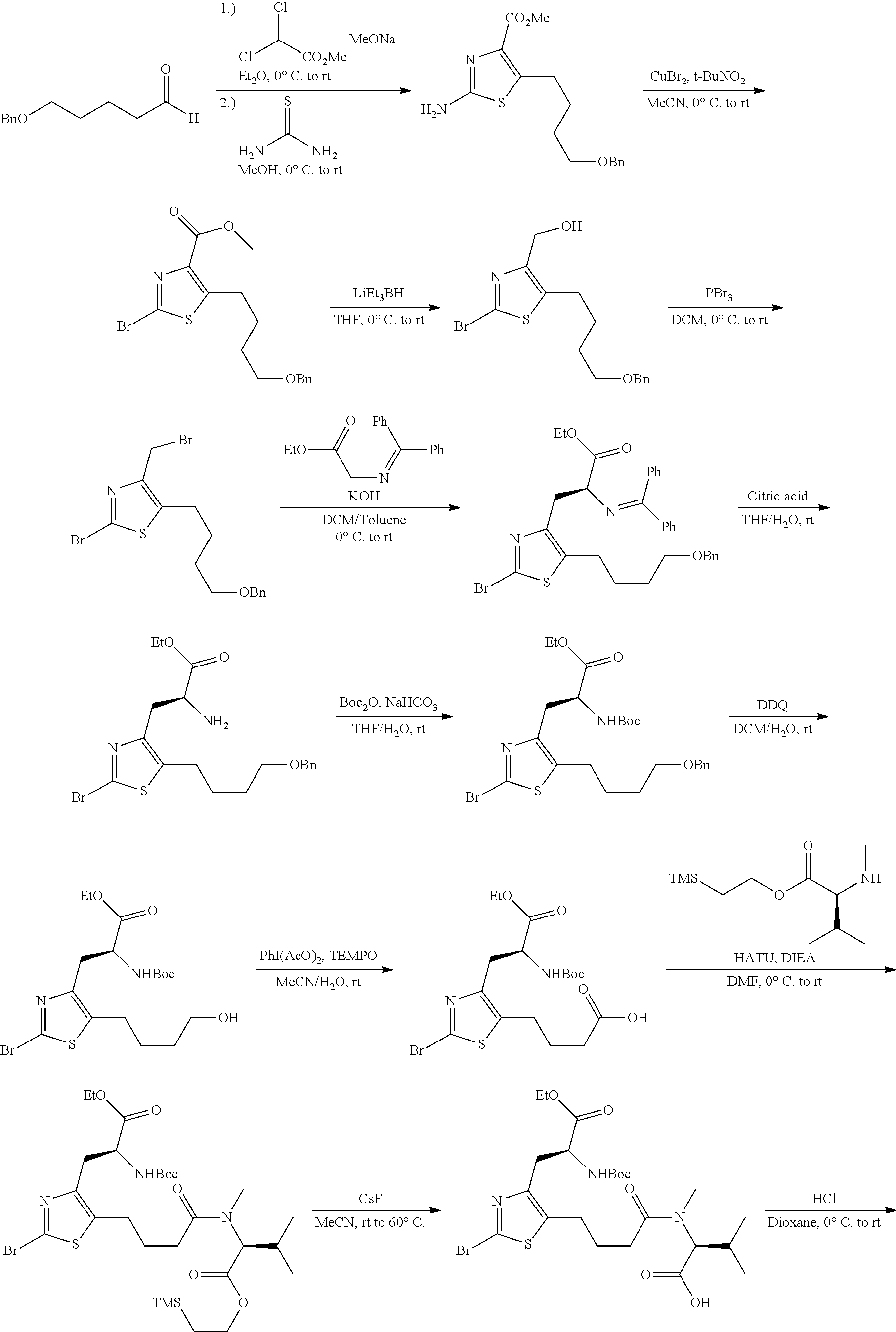

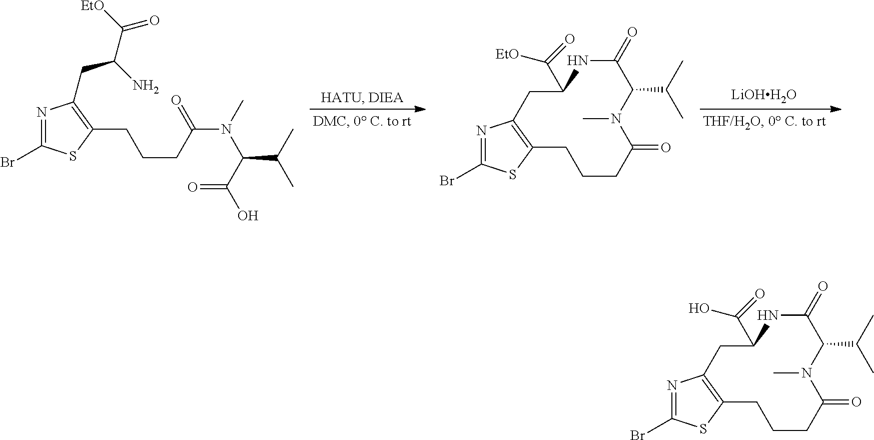

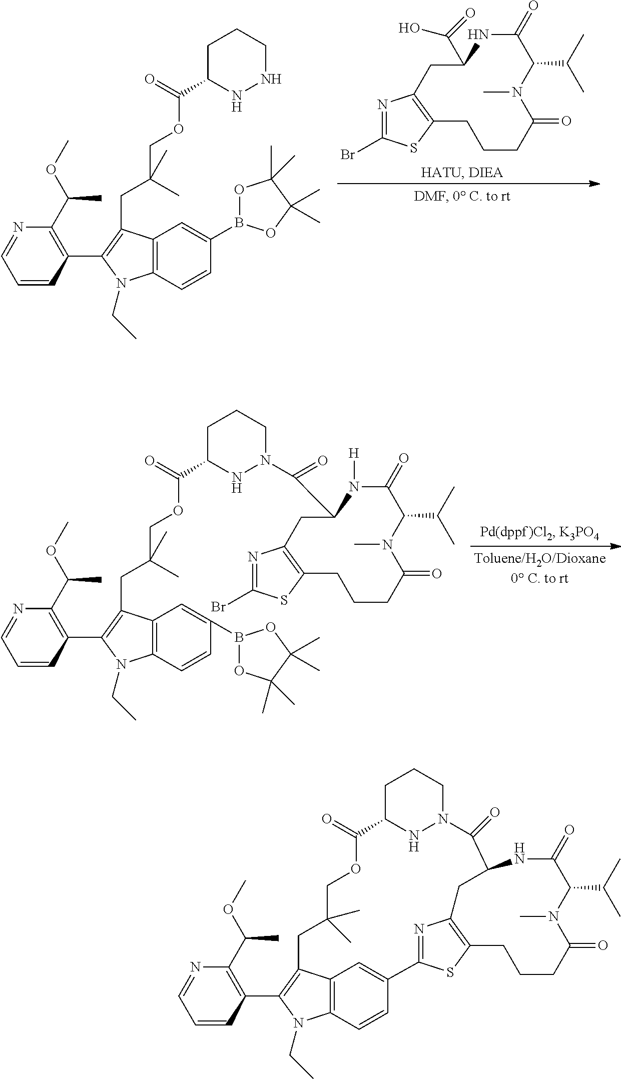

Step 1. To a stirred solution of 5-(benzyloxy)pentanal (50.0 g, 260 mmol) in Et₂O (400 mL) was added methyl dichloroacetate (74.4 g, 520 mmol) at 0° C. under an atmosphere of N₂. The resulting mixture was stirred for 10 minutes, after which time NaOMe (14.1 g, 260 mmol) was added dropwise at 0° C. The resulting mixture was stirred for 1 hour at room temperature under an atmosphere of N₂. The reaction mixture was washed with brine (3×100 mL), the combined aqueous phases were extracted with Et₂O (3×100 mL), and the combined organic extracts were concentrated under reduced pressure. The resulting material was diluted in methanol (400 mL) and thiourea (16.8 g, 221 mmol) in portions at 0° C. The resulting mixture was stirred for 4 hours at room temperature and was then concentrated under reduced pressure, acidified to pH 7 with sat. aq. NaHCO₃, extracted with DCM (3×100 mL), concentrated under reduced pressure, and purified by normal phase flash column chromatography to afford methyl 2-amino-5-(4-(benzyloxy)butyl)thiazole-4-carboxylate (37.0 g, 42% yield) as a yellow oil. LCMS (ESI) m/z: [M+H] calcd for C₁₆H₂₀N₂O₃S: 321.1; found 321.2.

Step 2. To a stirred mixture of methyl 2-amino-5-(4-(benzyloxy)butyl)thiazole-4-carboxylate (37.0 g, 115 mmol) and CuBr₂ (28.4 g, 127 mmol) in MeCN (400 mL) was added t-BuNO₂ (13.1 g, 127 mmol) in portions at 0° C. under an atmosphere of N₂. The resulting mixture was stirred for 2 hours at room temperature. The reaction mixture was quenched with sat. aq. NH₄Cl at 0° C., extracted with EtOAc (3×200 mL), washed with H₂O (200 mL), dried over anhydrous Na₂SO₄, filtered, concentrated under reduced pressure and purified by normal phase flash column chromatography to afford methyl 5-(4-(benzyloxy)butyl)-2-bromothiazole-4-carboxylate (22.0 g, 47% yield) as a yellow oil. LCMS (ESI) m/z: [M+H] calcd for C₁₆H₁₈BrNO₃S: 386.0; found 386.0.

Step 3. To a stirred solution of methyl 5-(4-(benzyloxy)butyl)-2-bromothiazole-4-carboxylate (22.0 g, 57.2 mmol) in THF (250 mL) was added with LiEt₃BH (114 mL, 114 mmol) in portions at 0° C. The resulting mixture was stirred for 2 hours at 0° C. under an atmosphere of N₂. The reaction mixture was quenched by the addition of concentrated HCl at 0° C., extracted with EtOAc (3×100 mL), washed with H₂O (3×100 mL), dried over anhydrous Na₂SO₄, filtered, concentrated under reduced pressure, and purified by by normal phase flash column chromatography to afford (5-(4-(benzyloxy)butyl)-2-bromothiazol-4-yl)methanol (20.0 g, 93% yield) as a yellow oil. LCMS (ESI) m/z: [M+H] calcd for C₁₅H₁₈BrNO₂S: 358.0; found 358.1.

Step 4. To a stirred solution of (5-(4-(benzyloxy)butyl)-2-bromothiazol-4-yl)methanol (20.0 g, 56.1 mmol) in DCM (200 mL) was added phosphorus tribromide (45.6 g, 168 mmol) in portions at 0° C. The resulting mixture was stirred for 2 hours at room temperature under an atmosphere of N₂. The reaction mixture was quenched by the slow addition of sat. aq. NaHCO₃, washed with brine (3×100 mL) of brine, concentrated under reduced pressure, and purified by normal phase flash column chromatography to afford 5-(4-(benzyloxy)butyl)-2-bromo-4-(bromomethyl)thiazole (8.0 g, 32% yield) as a yellow oil. LCMS (ESI) m/z: [M+H] calcd for C₁₅H₁₇Br₂NOS: 419.9; found 420.0.

Step 5. To a stirred solution of ethyl 2-((diphenylmethylene)amino)acetate (1.16 g, 1.91 mmol) and KOH (252 mg, 4.50 mmol) in DCM (70 mL) and toluene (30 mL) was added 5-(4-(benzyloxy)butyl)-2-bromo-4-(bromomethyl)thiazole (8.00 g, 19.1 mmol) in portions at 0° C. under an atmosphere of N₂. The resulting mixture was stirred for 18 hours at room temperature. The reaction mixture was diluted in H₂O, extracted with EtOAc (3×100 mL), washed with H₂O (300 mL), dried over anhydrous Na₂SO₄, filtered, and concentrated under reduced pressure to give ethyl (S)-3-(5-(4-(benzyloxy)butyl)-2-bromothiazol-4-yl)-2-((diphenylmethylene)amino)propanoate (6.0 g, crude) as a yellow oil. The material was taken directly to the next reaction without further purification. LCMS (ESI) m/z: [M+H] calcd for C₃₂H₃₃BrN₂O₃S: 607.0; found 607.1.

Step 6. A solution of ethyl (S)-3-(5-(4-(benzyloxy)butyl)-2-bromothiazol-4-yl)-2-((diphenylmethylene)amino)propanoate (6.0 g, crude) and citric acid (5.72 g, 2.98 mmol) in THF (60 mL) and H₂O (60 mL) was stirred for 18 hours at room temperature under an atmosphere of N₂. The reaction mixture was acidified to pH 7 by the addition of sat. aq. NaHCO₃, extracted with EtOAc (3×100 mL), washed with H₂O (200 mL), dried over anhydrous Na₂SO₄, filtered, and concentrated under reduced pressure to give ethyl (S)-2-amino-3-(5-(4-(benzyloxy)butyl)-2-bromothiazol-4-yl)propanoate (6.0 g, crude) as a yellow oil. The material was taken directly to the next reaction without further purification. LCMS (ESI) m/z: [M+H] calcd for C₁₉H₂₅BrN₂O₃S: 443.1; found 443.1.

Step 7. To a stirred mixture of ethyl (S)-2-amino-3-(5-(4-(benzyloxy)butyl)-2-bromothiazol-4-yl)propanoate (6.0 g, crude) and NaHCO₃ (5.71 g, 68.0 mmol) in THF (30 mL) and H₂O (30 mL) was added Boc₂O (5.93 g, 27.2 mmol) in portions at room temperature under an atmosphere of N₂. The reaction mixture was extracted with EtOAc (3×100 mL), washed with H₂O (100 mL), dried over anhydrous Na₂SO₄, filtered, concentrated under reduced and purified by normal phase flash column chromatography to afford ethyl (S)-3-(5-(4-(benzyloxy)butyl)-2-bromothiazol-4-yl)-2-((tert-butoxycarbonyl)amino)propanoate (7.00 g, 68% yield over 3 steps) as a yellow oil. LCMS (ESI) m/z: [M+H] calcd for C₂₄H₃₃BrN₂O₅S: 543.1; found 543.4.

Step 8. To a stirred mixture of ethyl (S)-3-(5-(4-(benzyloxy)butyl)-2-bromothiazol-4-yl)-2-((tert-butoxycarbonyl)amino)propanoate (7.00 g, 12.9 mmol) in DCM (80 mL) and H₂O (4 mL) was added DDQ (4.40 g, 19.4 mmol). The resulting mixture was stirred for 2 hours at room temperature under an atmosphere of N₂. The reaction mixture was then diluted with H₂O (100 mL), the organic extract was concentrated under reduced pressure, and the residue was purified by normal phase flash column chromatography to afford ethyl (S)-3-(2-bromo-5-(4-hydroxybutyl)thiazol-4-yl)-2-((tert-butoxycarbonyl)amino)propanoate (4.60 g, 75% yield) as a yellow oil. LCMS (ESI) m/z: [M+H] calcd for C₁₇H₂₇BrN₂O₅S: 451.1; found 451.1.

Step 9. To a stirred solution of ethyl (S)-3-(2-bromo-5-(4-hydroxybutyl)thiazol-4-yl)-2-((tert-butoxycarbonyl)amino)propanoate (4.60 g, 10.2 mmol) and PhI(OAc)₂ (9.85 g, 30.6 mmol) in H₂O (50 mL) and ACN (50 mL) was added TEMPO (320 mg, 2.04 mmol) in portions at room temperature under an atmosphere of N₂. The resulting mixture was stirred for 18 hours at room temperature. The resulting mixture was diluted in DCM (50 mL), washed with H₂O (50 mL), concentrated under reduced pressure, and purified by normal phase flash column chromatography to afford (S)-4-(2-bromo-4-(2-((tert-butoxycarbonyl)amino)-3-ethoxy-3-oxopropyl)thiazol-5-yl)butanoic acid (3.0 g, 60% yield) as a yellow solid. LCMS (ESI) m/z: [M+H] calcd for C₁₇H₂₅BrN₂O₅S: 465.1; found 465.1.

Step 10. To a stirred mixture of (S)-4-(2-bromo-4-(2-((tert-butoxycarbonyl)amino)-3-ethoxy-3-oxopropyl)thiazol-5-yl)butanoic acid (3.00 g, 6.45 mmol) and 2-(trimethylsilyl)ethyl (2S)-3-methyl-2-(methylamino)butanoate (2.24 g, 9.67 mmol) in DMF (30 mL) were added DIEA (4.17 g, 32.2 mmol) and HATU (3.59 g, 12.9 mmol) in portions at 0° C. under an atmosphere of N₂. The resulting mixture was stirred for 1 hour at room temperature. The reaction mixture was diluted in H₂O (30 mL), extracted with DCM (3×30 mL), washed with brine (5×10 mL), concentrated under reduced pressure, and purified by normal phase flash column chromatography to afford 2-(trimethylsilyl)ethyl N-(4-(2-bromo-4-((S)-2-((tert-butoxycarbonyl)amino)-3-ethoxy-3-oxopropyl)thiazol-5-yl)butanoyl)-N-methyl-L-valinate (2.40 g, 52% yield) as a yellow solid. LCMS (ESI) m/z: [M+Na] calcd for C₂₈H₄₈BrN₃O₇SSi: 702.2; found 702.2.

Step 11. A mixture of 2-(trimethylsilyl)ethyl N-(4-(2-bromo-4-((S)-2-((tert-butoxycarbonyl)amino)-3-ethoxy-3-oxopropyl)thiazol-5-yl)butanoyl)-N-methyl-L-valinate (2.40 g, 3.54 mmol) and CsF (1.61 g, 10.6 mmol) in MeCN (30 mL) was stirred for 5 minutes at room temperature under an atmosphere of N₂. The resulting mixture was then stirred for 2 hour at 60° C. The reaction mixture was filtered, the filter cake was washed with MeCN (3×20 mL), and the filtrate was concentrated under reduced pressure to give N-(4-(2-bromo-4-((S)-2-((tert-butoxycarbonyl)amino)-3-ethoxy-3-oxopropyl)thiazol-5-yl)butanoyl)-N-methyl-L-valine (2.0 g, crude) as a yellow oil. This material was taken directly to the next reaction without further purification. LCMS (ESI) m/z: [M+H] calcd for C₂₃H₃₆BrN₃O₇S: 580.1; found 580.1.

Step 12. N-(4-(2-bromo-4-((S)-2-((tert-butoxycarbonyl)amino)-3-ethoxy-3-oxopropyl)thiazol-5-yl)butanoyl)-N-methyl-L-valine (2.0 g, crude) was suspended in a solution of HCl in 1,4-dioxane (1.0 M, 20.0 mL, 20.0 mmol) at 0° C. under an atmosphere of N₂. The resulting mixture was stirred for 1 hour at room temperature and was then concentrated under reduced pressure to give N-(4-(4-((S)-2-amino-3-ethoxy-3-oxopropyl)-2-bromothiazol-5-yl)butanoyl)-N-methyl-L-valine (2.0 g, crude) as a yellow solid. This material was taken directly to the next reaction without further purification. LCMS (ESI) m/z: [M+H] calcd for C₁₈H₂₈BrN₃O₅S: 478.1; found 478.1.

Step 13. To a stirred solution of N-(4-(4-((S)-2-amino-3-ethoxy-3-oxopropyl)-2-bromothiazol-5-yl)butanoyl)-N-methyl-L-valine (2.0 g, crude) in DMF (20 mL) were added DIEA (2.70 g, 20.9 mmol) followed by HATU (3.18 g, 8.36 mmol) dropwise at 0° C. under an atmosphere of N₂. The reaction mixture was diluted in H₂O (20 mL), extracted with DCM (3×20 mL), washed with H₂O (3×10 mL), concentrated under reduced pressure, and purified by normal phase flash column chromatography to afford ethyl (5S,8S)-2-bromo-8-isopropyl-9-methyl-7,10-dioxo-4,5,6,7,8,9,10,11,12,13-decahydrothiazolo[4,5-g][1,4]diazacyclododecine-5-carboxylate (450 mg, 28% over 3 steps) as a yellow solid. LCMS (ESI) m/z: [M+H] calcd for C₁₈H₂₆BrN₃O₄S: 462.1; found 462.0.

Step 14. To a stirred mixture of ethyl (5S,8S)-2-bromo-8-isopropyl-9-methyl-7,10-dioxo-4,5,6,7,8,9,10,11,12,13-decahydrothiazolo[4,5-g][1,4]diazacyclododecine-5-carboxylate (450 mg, 0.98 mmol) in THF (5 mL) and H₂O (5 mL) was added LiOH·H₂O (83.2 mg, 3.48 mmol) at 0° C. under an atmosphere of N₂. The resulting mixture was stirred for 1 hour at room temperature. The reaction mixture was then acidified to pH 5 by the addition of aq. HCl, extracted with DCM (3×10 mL), washed with H₂O (10 mL), dried over anhydrous Na₂SO₄, filtered, and concentrated under reduced pressure to give (5S,8S)-2-bromo-8-isopropyl-9-methyl-7,10-dioxo-4,5,6,7,8,9,10,11,12,13-decahydrothiazolo[4,5-g][1,4]diazacyclododecine-5-carboxylic acid (460 mg, crude) as a yellow solid. This material was taken directly to the next reaction without further purification. LCMS (ESI) m/z: [M+H] calcd for C₁₆H₂₂BrN₃O₄S: 434.1; found 434.1.

Step 15. To a stirred solution of 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (S)-hexahydropyridazine-3-carboxylate (503 mg, 8.33 mmol) and (5S,8S)-2-bromo-8-isopropyl-9-methyl-7,10-dioxo-4,5,6,7,8,9,10,11,12,13-decahydrothiazolo[4,5-g][1,4]diazacyclododecine-5-carboxylic acid (240 mg, crude) in DMF (5 mL) were added DIEA (717 mg, 5.55 mmol) and HATU (422 mg, 1.11 mmol) in portions at 0° C. under an atmosphere of N₂. The resulting mixture was stirred for 1 hour at room temperature. The reaction mixture was diluted in H₂O (20 mL), extracted with DCM (3×20 mL), washed with brine (3×10 mL), concentrated under reduced pressure, and purified by normal phase flash column chromatography to afford 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (S)-1-((5S,8S)-2-bromo-8-isopropyl-9-methyl-7,10-dioxo-4,5,6,7,8,9,10,11,12,13-decahydrothiazolo[4,5-g][1,4]diazacyclododecine-5-carbonyl)hexahydropyridazine-3-carboxylate (300 mg, 58% yield over 2 steps) as a yellow solid. LCMS (ESI) m/z: [M+H] calcd for C₅₀H₆₉BBrN₇O₈S: 1020.4; found 1020.4.

Step 16. To a stirred solution of 3-(1-ethyl-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropyl (S)-1-((5S,8S)-2-bromo-8-isopropyl-9-methyl-7,10-dioxo-4,5,6,7,8,9,10,11,12,13-decahydrothiazolo[4,5-g][1,4]diazacyclododecine-5-carbonyl)hexahydropyridazine-3-carboxylate (300 mg, 0.294 mmol) and Pd(dppf)Cl₂ (21.5 mg, 0.029 mmol) in toluene (15 mL), H₂O (5 mL), and 1,4-dioxane (5 mL) was added K₃PO₄ (156 mg, 0.735 mmol) in portions at room temperature under an atmosphere of N₂. The resulting mixture was stirred for 2 hour at 80° C. The reaction mixture was then diluted in H₂O (10 mL), extracted with EtOAc (3×10 mL), concentrated under reduced pressure, and purified by reversed-phase prep-HPLC to afford (2²Z,2³aZ,2⁵S,2⁸S,4³S)-1¹-ethyl-2⁸-isopropyl-1²-(2-((S)-1-methoxyethyl)pyridin-3-yl)-2⁹,8,8-trimethyl-2⁴,2⁵,2⁶,2⁷,2⁸,2⁹,2¹⁰,2¹¹,2¹²,2¹³, 4¹,4²,4³,4⁴,4⁵,4⁶-hexadecahydro-1¹H-6-oxa-2(2,5)- thiazolo[4,5-g][1,4]diazacyclododecina-1(5,3)-indola-4(1,3)-pyridazinacyclononaphane-2⁷,2¹⁰,3,5-tetraone (35.3 mg, 18% yield) as a white solid. LCMS (ESI) m/z: [M+H] calcd for C₄₄H₅₇BrN₇O₆S: 812.4; found 812.7; ¹H NMR (400 MHz, DMSO-d₆) δ 8.79-8.71 (m, 2H), 8.49-8.43 (m, 1H), 7.78-7.70 (m, 1H), 7.65-7.55 (m, 1H), 7.57-7.49 (m, 2H), 5.53-5.52 (m, 1H), 5.10-5.00 (m, 1H), 4.31-4.29 (m, 4H), 4.19-4.13 (m, 1H), 4.01-3.99 (m, 1H), 3.60-3.50 (m, 2H), 3.35-3.30 (m, 2H) 3.09-2.95 (m, 1H), 3.13-2.95 (m, 4H), 2.87-2.85 (m, 6H), 2.33-1.80 (m, 4H), 1.81 (s, 2H), 1.80-1.62 (m, 1H), 1.60-1.21 (m, 3H) 1.15-1.11 (m, 1H), 0.99-0.97 (m, 2H), 0.96-0.69 (m, 10H), 0.35-0.27 (m, 3H).

### Synthesis of Compound A54—(9S,15S,18S,20aS,23aS)-3-(5-(4-cyclopropylpiperazin-1-yl)-2-((S)-1-methoxyethyl)pyridin-3-yl)-2-ethyl-18-isopropyl-5,5,19-trimethyl-21-((S)-1-methylpyrrolidine-3-carbonyl)-2,4,5,6,9,10,11,12,15,16,18,19,20a,21,22,23,23a,24-octadecahydro-8H,14H-9,13-epimino-1,31-etheno-15,28-methano-26,30-(metheno)dipyrrolo[3,2-c:3′,4′-v][1,18]dioxa[6,9,12]triazacyclotriacontine-8,14,17,20-tetraone

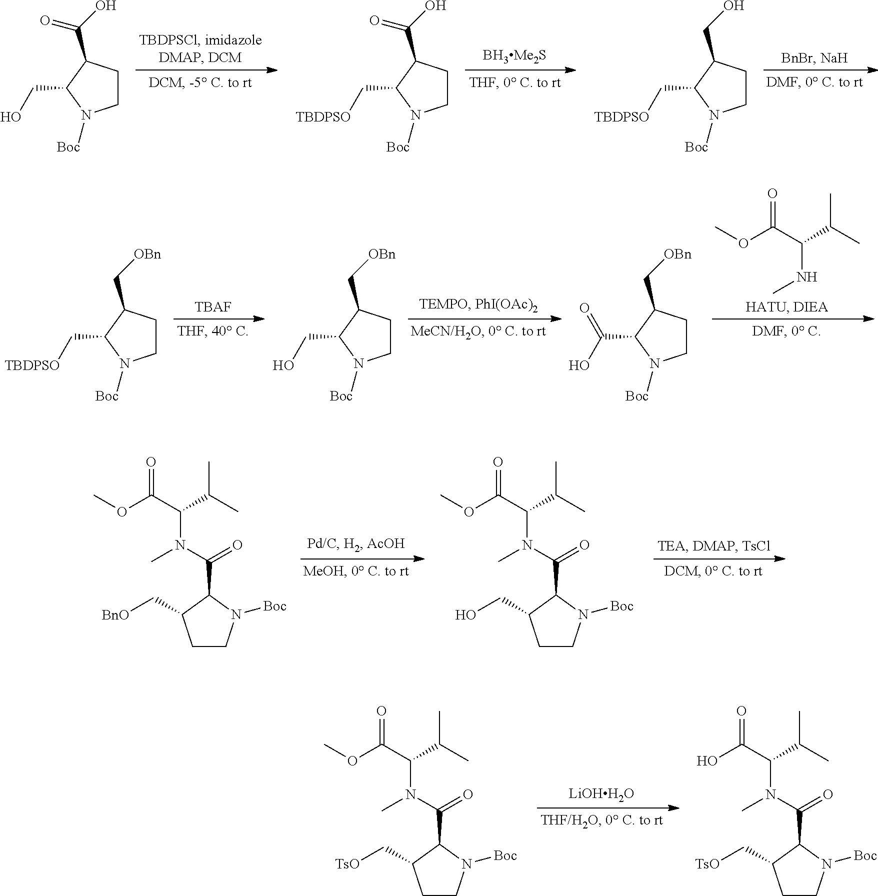

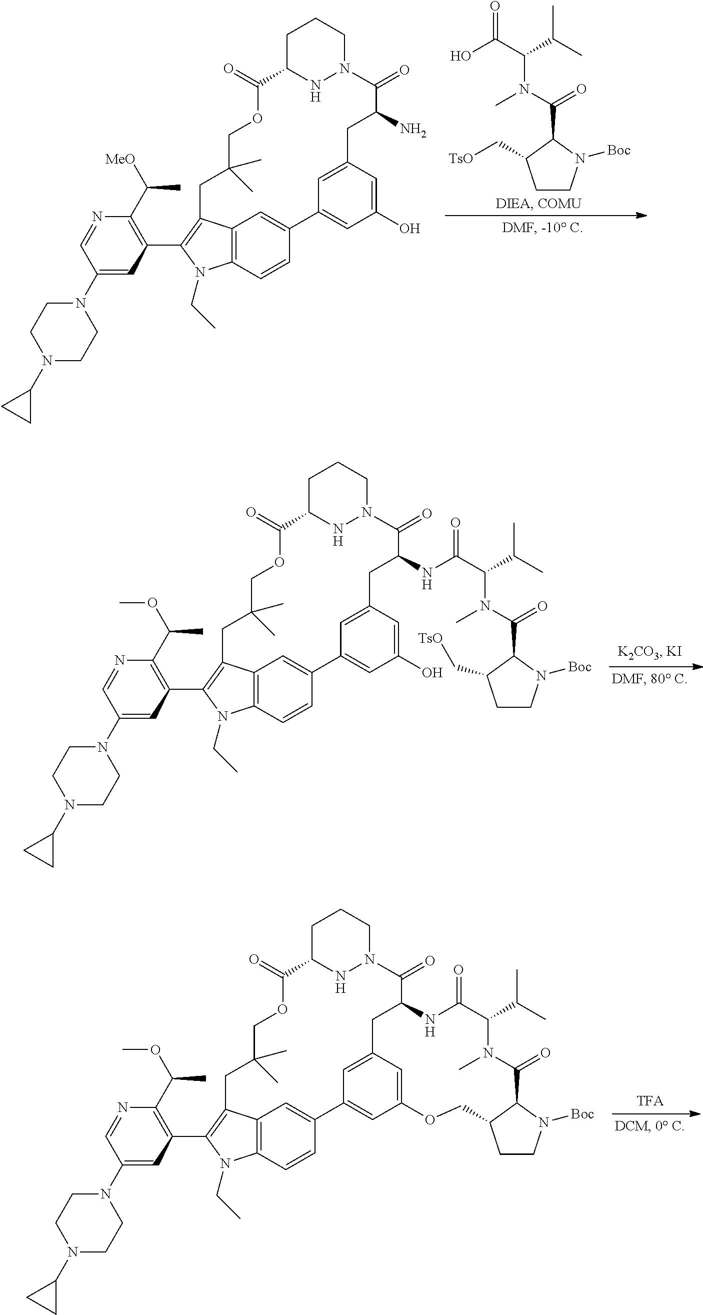

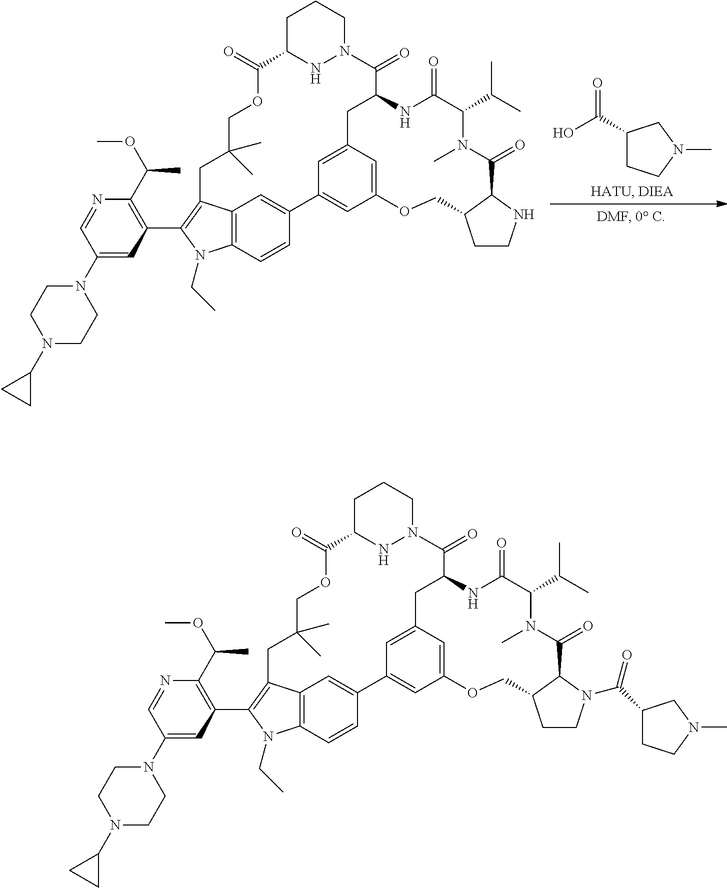

Step 1. To a stirred solution of (2S,3S)-1-(tert-butoxycarbonyl)-2-(hydroxymethyl)pyrrolidine-3-carboxylic acid (6.00 g, 24.5 mmol) in DCM (40 mL) was added imidazole (4.41 g, 61.2 mmol) in portions at −5° C. under an atmosphere of argon. To this mixture was added a solution of TBDPSCI (6.05 g, 22.0 mmol) in DCM (20 mL) and DMAP (30.0 mg, 0.245 mmol) dropwise at −5° C. The resulting mixture was stirred for 16 h at room temperature. The reaction mixture was then acidified to pH 6 by the additional 1 M aq. HCl, extracted with EtOAc (3×400 mL), treated with brine (3×400 mL), dried over anhydrous Na₂SO₄, filtered, concentrated under reduced pressure, and purified by reversed-phase flash column chromatography to afford (2S,3S)-1-(tert-butoxycarbonyl)-2-{[(tert-butyldiphenylsilyl)oxy]methyl}pyrrolidine-3-carboxylic acid (2.68 g, 23% yield) as a yellow oil. LCMS (ESI) m/z: [M+H] calcd for C₂₇H₃₇NO₅Si: 484.2; found 484.1.

Step 2. To a stirred solution of (2S,3S)-1-(tert-butoxycarbonyl)-2-{[(tert-butyldiphenylsilyl)oxy]methyl}pyrrolidine-3-carboxylic acid (3.54 g, 7.32 mmol) in THF (21 mL) was added BH₃·Me₂S (11.0 mL, 22.0 mmol) dropwise at 0° C. under an atmosphere of argon. The resulting mixture was stirred for 16 h at room temperature. The reaction mixture was quenched by the addition of MeOH at 0° C., diluted with brine (300 mL), extracted with EtOAc (3×200 mL), treated with brine (3×200 mL), dried over anhydrous Na₂SO₄, filtered, concentrated under reduced pressure, and purified by reversed-phase flash column chromatography to afford tert-butyl (2S,3S)-2-{[(tert-butyldiphenylsilyl)oxy]methyl}-3-(hydroxymethyl)pyrrolidine-1-carboxylate (1.80 g, 52% yield) as a yellow oil. LCMS (ESI) m/z: [M+H] calcd for C₂₇H₃₉NO₄Si: 470.3; found 470.1.

Step 3. To a stirred solution of tert-butyl (2S,3S)-2-{[(tert-butyldiphenylsilyl)oxy]methyl}-3-(hydroxymethyl)pyrrolidine-1-carboxylate (3.20 g, 6.81 mmol) in DMF (32 mL) was added NaH (570 mg, 23.8 mmol) in portions at 0° C. under an atmosphere of argon. This mixture was stirred for 1 h at room temperature, after which time benzyl bromide (4.66 g, 27.3 mmol) was added in portions at 0° C. The resulting mixture was stirred for 3 h at room temperature. The reaction mixture was quenched by the addition of sat. aq. NH₄Cl at 0° C., extracted with DCM (3×300 mL), treated with brine (3×300 mL), dried over anhydrous Na₂SO₄, filtered, concentrated under reduced pressure, and purified by reversed-phase flash column chromatography to afford tert-butyl (2S,3S)-3-[(benzyloxy)methyl]-2-{[(tert-butyldiphenylsilyl)oxy]methyl}pyrrolidine-1-carboxylate (2.74 g, 72% yield) as a yellow oil. LCMS (ESI) m/z: [M+H] calcd for C₃₄H₄₅NO₄Si: 560.3; found 560.3.

Step 4. tert-butyl (2S,3S)-3-[(benzyloxy)methyl]-2-{[(tert-butyldiphenylsilyl)oxy]methyl}pyrrolidine-1-carboxylate (2.74 g, 4.89 mmol) was added to a stirred 1 M solution of TBAF in THF (27.4 mL, 27.4 mmol) at room temperature. The resulting mixture was stirred for 2 h at 40° C. The reaction mixture was then diluted with brine (300 mL), extracted with EtOAc (3×300 mL), treated with brine (3×300 mL), dried over anhydrous Na₂SO₄, filtered, concentrated under reduced pressure, and purified by reversed-phase flash column chromatography to afford tert-butyl (2S,3S)-3-[(benzyloxy)methyl]-2-(hydroxymethyl)pyrrolidine-1-carboxylate (1.12 g, 71% yield) as a yellow oil. LCMS (ESI) m/z: [M+H] calcd for C₁₈H₂₇NO₄: 322.2; found 322.2.

Step 5. To a stirred solution of tert-butyl (2S,3S)-3-[(benzyloxy)methyl]-2-(hydroxymethyl)pyrrolidine-1-carboxylate (100 mg, 0.311 mmol) in H₂O (5.6 mL) and MeCN (5.60 ml) were added TEMPO (160 mg, 1.05 mmol) and PhI(OAc)₂ (2.47 g, 7.67 mmol) in portions at 0° C. under an atmosphere of argon. The resulting mixture was stirred for 2 h at room temperature. The reaction mixture was filtered, the filter cake was washed with MeOH, the filtrate was concentrated under reduced pressure, and the crude product was purified by reversed-phase flash column chromatography to afford (2S,3S)-3-[(benzyloxy)methyl]-1-(tert-butoxycarbonyl)pyrrolidine-2-carboxylic acid (870 mg, 74% yield) as a yellow oil. LCMS (ESI) m/z: [M+H] calcd for C₁₈H₂₅NO₅: 336.2; found 336.1.

Step 6. To a stirred solution of methyl (2S)-3-methyl-2-(methylamino)butanoate (515 mg, 3.55 mmol) in DMF (7.0 mL) were added DIEA (1.64 g, 12.7 mmol) (2S,3S)-3-[(benzyloxy)methyl]-1-(tert-butoxycarbonyl)pyrrolidine-2-carboxylic acid (850 mg, 2.53 mmol) in portions at 0° C. To this stirred mixture was added HATU (1.25 g, 3.29 mmol) in DMF (1.5 mL) dropwise at 0° C. The resulting mixture was stirred for 2 h at 0° C. The reaction mixture was then diluted with brine (100 mL), extracted with EtOAc (3×100 mL), treated with brine (3×100 mL), dried over anhydrous Na₂SO₄, filtered, concentrated under reduced pressure, and purified by normal phase flash column chromatography to afford tert-butyl (2S,3S)-3-[(benzyloxy)methyl]-2-{[(2S)-1-methoxy-3-methyl-1-oxobutan-2-yl](methyl)carbamoyl}pyrrolidine-1-carboxylate (1.08 g, 92% yield) as a white solid. LCMS (ESI) m/z: [M+H] calcd for C₂₅H₃₈N₂O₆: 463.3; found 463.3.

Step 7. To a stirred solution of tert-butyl (2S,3S)-3-[(benzyloxy)methyl]-2-{[(2S)-1-methoxy-3-methyl-1-oxobutan-2-yl](methyl)carbamoyl}pyrrolidine-1-carboxylate (1.08 g, 2.34 mmol) in MeOH (9.0 mL) were added 10% Pd/C (0.50 g) and AcOH (1.00 mL, 17.5 mmol) in portions at 0° C. under an atmosphere of H₂. The resulting mixture was stirred for 16 h at room temperature. The reaction mixture was filtered, the filter cake washed with MeOH, and the filtrate concentrated under reduced pressure to give tert-butyl (2S,3S)-3-(hydroxymethyl)-2-{[(2S)-1-methoxy-3-methyl-1-oxobutan-2-yl](methyl)carbamoyl}pyrrolidine-1-carboxylate (660 mg, crude) as a yellow oil. This material was taken directly to the next step without further purification. LCMS (ESI) m/z: [M+H] calcd for C₁₈H₃₂N₂O₆: 373.2; found 373.2.

Step 8. To a stirred solution of tert-butyl (2S,3S)-3-(hydroxymethyl)-2-{[(2S)-1-methoxy-3-methyl-1-oxobutan-2-yl](methyl)carbamoyl}pyrrolidine-1-carboxylate (690 mg, crude) in DCM (3.5 mL) were added TEA (1.12 g, 11.1 mmol) and DMAP (22.6 mg, 0.185 mmol), followed by a solution of TsCl (706 mg, 3.71 mmol) in DMF (3.5 mL) dropwise at 0° C. under an atmosphere of argon. The resulting mixture was stirred for 16 h at room temperature. The reaction mixture was then acidified to pH 6 by the addition of sat. aq. NH₄Cl, extracted with EtOAc (3×60 mL), treated with brine (3×60 mL), dried over anhydrous Na₂SO₄, filtered, concentrated under reduced pressure, and purified by normal phase flash column chromatography to afford tert-butyl (2S,3S)-2-{[(2S)-1-methoxy-3-methyl-1-oxobutan-2-yl](methyl)carbamoyl}-3-{[(4-methylbenzenesulfonyl)oxy]methyl}pyrrolidine-1-carboxylate (820 mg, 64% yield over 2 steps) as a yellow oil. LCMS (ESI) m/z: [M+H] calcd for C₂₅H₃₈N₂O₈S: 527.2; found 527.4.

Step 9. To a stirred solution of tert-butyl (2S,3S)-2-{[(2S)-1-methoxy-3-methyl-1-oxobutan-2-yl](methyl)carbamoyl}-3-{[(4-methylbenzenesulfonyl)oxy]methyl}pyrrolidine-1-carboxylate (810 mg, 1.54 mmol) in THF (4.0 mL) and H₂O (4.0 mL) were added LiOH·H₂O (73.7 mg, 3.08 mmol) in portions at 0° C. under an atmosphere of argon. The resulting mixture was stirred for 16 h at room temperature. The reaction mixture was then acidified to pH 6 by the addition of sat. aq. NH₄Cl, extracted with EtOAc (3×30 mL), treated with brine (3×30 mL), dried over anhydrous Na₂SO₄, filtered, and concentrated under reduced pressure to afford (2S)-2-{1-[(2S,3S)-1-(tert-butoxycarbonyl)-3-{[(4-methylbenzenesulfonyl)oxy]methyl}pyrrolidin-2-yl]-N-methylformamido}-3-methylbutanoic acid (423 mg, crude) as a yellow solid. This material was taken directly to the next reaction without further purification. LCMS (ESI) m/z: [M+H] calcd for C₂₄H₃₆N₂O₈S: 513.2; found 513.2.

Step 10. To a stirred solution of (6³S,4S)-4-amino-1²-(5-(4-cyclopropylpiperazin-1-yl)-2-((S)-1-methoxyethyl)pyridin-3-yl)-11-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (615 mg, 0.806 mmol) in DMF (6.0 mL) were added DIEA (4.17 g, 32.2 mmol) and (2S)-2-{1-[(2S,3S)-1-(tert-butoxycarbonyl)-3-{[(4-methylbenzenesulfonyl)oxy]methyl}pyrrolidin-2-yl]-N-methylformamido}-3-methylbutanoic acid (536.89 mg, crude) followed by COMU (518 mg, 1.21 mmol) in portions at −10° C. under an atmosphere of argon. The resulting mixture was stirred for 2 hours at −10° C. The reaction mixture was quenched with sat. aq. NH₄Cl (60 mL) at 0° C., extracted with EtOAc (3×60 mL), washed with brine (3×60 mL), dried over anhydrous Na₂SO₄, filtered, concentrated under reduced pressure, and purified by normal phase flash column chromatography to afford tert-butyl (2S,3S)-2-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-3-((tosyloxy)methyl)pyrrolidine-1-carboxylate (1.30 g, crude) as a white solid. This material was taken directly to the next reaction without further purification. LCMS (ESI) m/z: [M+H] calcd for C₆₈H₉₁N₉O₁₂S: 1258.7; found 1258.6.

Step 11. To a stirred solution of tert-butyl (2S,3S)-2-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-3-((tosyloxy)methyl)pyrrolidine-1-carboxylate (1.30 g, crude) in DMF (130 mL) were added K₂CO₃ (1.43 g, 10.3 mmol) and KI (170 mg, 1.03 mmol) in portions at room temperature under an atmosphere of argon. The resulting mixture was stirred at 80° C. for 5 hours. The reaction mixture was then quenched with sat. aq. NH₄Cl (500 mL) at 0° C., extracted with EtOAc (3×500 mL), washed with brine (3×500 mL), dried over anhydrous Na₂SO₄, filtered, concentrated under reduced pressure, and purified by normal phase prep-TLC to afford tert-butyl (9S,15S,18S,20aS,23aS)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,20a,22,23,23a,24-icosahydro-8H,21H-9,13-epimino-1,31-etheno-15,28-methano-26,30-(metheno)dipyrrolo[3,2-c:3′,4′-v][1,18]dioxa[6,9,12]triazacyclotriacontine-21-carboxylate (564 mg, 64% yield over 2 steps) as a white solid. LCMS (ESI) m/z: [M+H] calcd for C₆₁H₈₃N₉O₉: 1086.6; found 1086.5.

Step 12. To a stirred solution of tert-butyl (9S,15S,18S,20aS,23aS)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,20a,22,23,23a,24-icosahydro-8H,21H-9,13-epimino-1,31-etheno-15,28-methano-26,30-(metheno)dipyrrolo[3,2-c:3′,4′-v][1,18]dioxa[6,9,12]triazacyclotriacontine-21-carboxylate (564 mg, 0.519 mmol) in DCM (4.2 mL) was added TFA (1.40 mL, 19.0 mmol) dropwise at 0° C. under an atmosphere of argon. The resulting mixture was stirred at 0° C. for 30 minutes. The reaction mixture was concentrated under reduced pressure, basified to pH 8 with sat. aq. NaHCO₃, extracted with EtOAc (3×50 mL), washed with brine (3×50 mL), dried over anhydrous Na₂SO₄, filtered, and concentrated under reduced pressure to give (9S,15S,18S,20aS,23aS)-3-(5-(4-cyclopropylpiperazin-1-yl)-2-((S)-1-methoxyethyl)pyridin-3-yl)-2-ethyl-18-isopropyl-5,5,19-trimethyl-2,4,5,6,9,10,11,12,15,16,18,19,20a,21,22,23,23a,24-octadecahydro-8H,14H-9,13-epimino-1,31-etheno-15,28-methano-26,30-(metheno)dipyrrolo[3,2-c:3′,4′-v][1,18]dioxa[6,9,12]triazacyclotriacontine-8,14,17,20-tetraone (470 mg, crude) as a yellow oil. This material was taken directly to the next reaction without further purification. LCMS (ESI) m/z: [M+H] calcd for C₅₆H₇₅N₉O₇: 986.6; found 986.7.

Step 13. To a stirred solution of (9S,15S,18S,20aS,23aS)-3-(5-(4-cyclopropylpiperazin-1-yl)-2-((S)-1-methoxyethyl)pyridin-3-yl)-2-ethyl-18-isopropyl-5,5,19-trimethyl-2,4,5,6,9,10,11,12,15,16,18,19,20a,21,22,23,23a,24-octadecahydro-8H,14H-9,13-epimino-1,31-etheno-15,28-methano-26,30-(metheno)dipyrrolo[3,2-c:3′,4′-v][1,18]dioxa[6,9,12]triazacyclotriacontine-8,14,17,20-tetraone (50 mg, crude) in DMF (0.3 mL) were added DIEA (262 mg, 2.04 mmol) and (3S)-1-methylpyrrolidine-3-carboxylic acid (13.1 mg, 0.102 mmol) in portions at 0° C. under an atmosphere of argon. To the above mixture was added a solution of HATU (25.1 mg, 0.066 mmol) in DMF (0.20 mL) dropwise at 0° C. The resulting mixture was stirred at 0° C. for 2 hours. The reaction mixture was then diluted with brine (30 mL), extracted with EtOAc (3×20 mL), washed with brine (3×20 mL), dried over anhydrous Na₂SO₄, filtered, concentrated under reduced pressure, and purified by reversed-phase prep-HPLC to afford (9S,15S,18S,20aS,23aS)-3-(5-(4-cyclopropylpiperazin-1-yl)-2-((S)-1-methoxyethyl)pyridin-3-yl)-2-ethyl-18-isopropyl-5,5,19-trimethyl-21-((S)-1-methylpyrrolidine-3-carbonyl)-2,4,5,6,9,10,11,12,15,16,18,19,20a,21,22,23,23a,24-octadecahydro-8H,14H-9,13-epimino-1,31-etheno-15,28-methano-26,30-(metheno)dipyrrolo[3,2-c:3′,4′-v][1,18]dioxa[6,9,12]triazacyclotriacontine-8,14,17,20-tetraone (20.1 mg, 33% yield over 2 steps) as a white solid. LCMS (ESI) m/z: [M+H] calcd for C₆₂H₈₄N₁₀O₈: 1097.7; found 1097.7; ¹H-NMR (400 MHz, DMSO-d₆) δ 8.51 (d, J=8.6 Hz, 1H), 8.45 (d, J=2.8 Hz, 1H), 7.91 (s, 1H), 7.64-7.53 (m, 2H), 7.29 (d, J=9.8 Hz, 1H), 7.18 (d, J=2.9 Hz, 1H), 7.13 (s, 1H), 6.65 (d, J=10.8 Hz, 1H), 5.24-5.07 (m, 2H), 4.80 (d, J=11.3 Hz, 1H), 4.47 (d, J=8.1 Hz, 1H), 4.34-4.02 (m, 8H), 3.87 (s, 1H), 3.75-3.58 (m, 2H), 3.54-3.44 (m, 1H), 3.23 (s, 5H), 3.18 (d, J=8.7 Hz, 4H), 3.10-2.90 (m, 3H), 2.87-2.71 (m, 4H), 2.71-2.64 (m, 6H), 2.44-2.36 (m, 2H), 2.34-2.26 (m, 3H), 2.22 (s, 3H), 2.17-2.03 (m, 3H), 2.01-1.89 (m, 2H), 1.88-1.72 (m, 4H), 1.66 (d, J=3.9 Hz, 2H), 1.53 (d, J=12.9 Hz, 2H), 1.35 (s, 1H), 1.32 (d, J=6.1 Hz, 3H), 1.24 (s, 2H), 0.99-0.83 (m, 12H), 0.45 (t, J=6.4 Hz, 3H), 0.40 (s, 2H), 0.34 (s, 3H).

### Synthesis of Compound A27—(4aR,7S,10S,16S,33aR)-22-(5-(4-cyclopropylpiperazin-1-yl)-2-((S)-1-methoxyethyl)pyridin-3-yl)-23-ethyl-7-isopropyl-6,20,20-trimethyl-2,3,4,4a,6,7,9,10,15,16,19,20,21,23,33,33a-hexadecahydro-13H-12,16-epimino-24,26-etheno-10,29-methano-27,31-(metheno)pyrido[3,4-c]pyrrolo[3,4-v][1,18]dioxa[6,9,12]triazacyclotriacontine-5,8,11,17(1H,14H)-tetraone

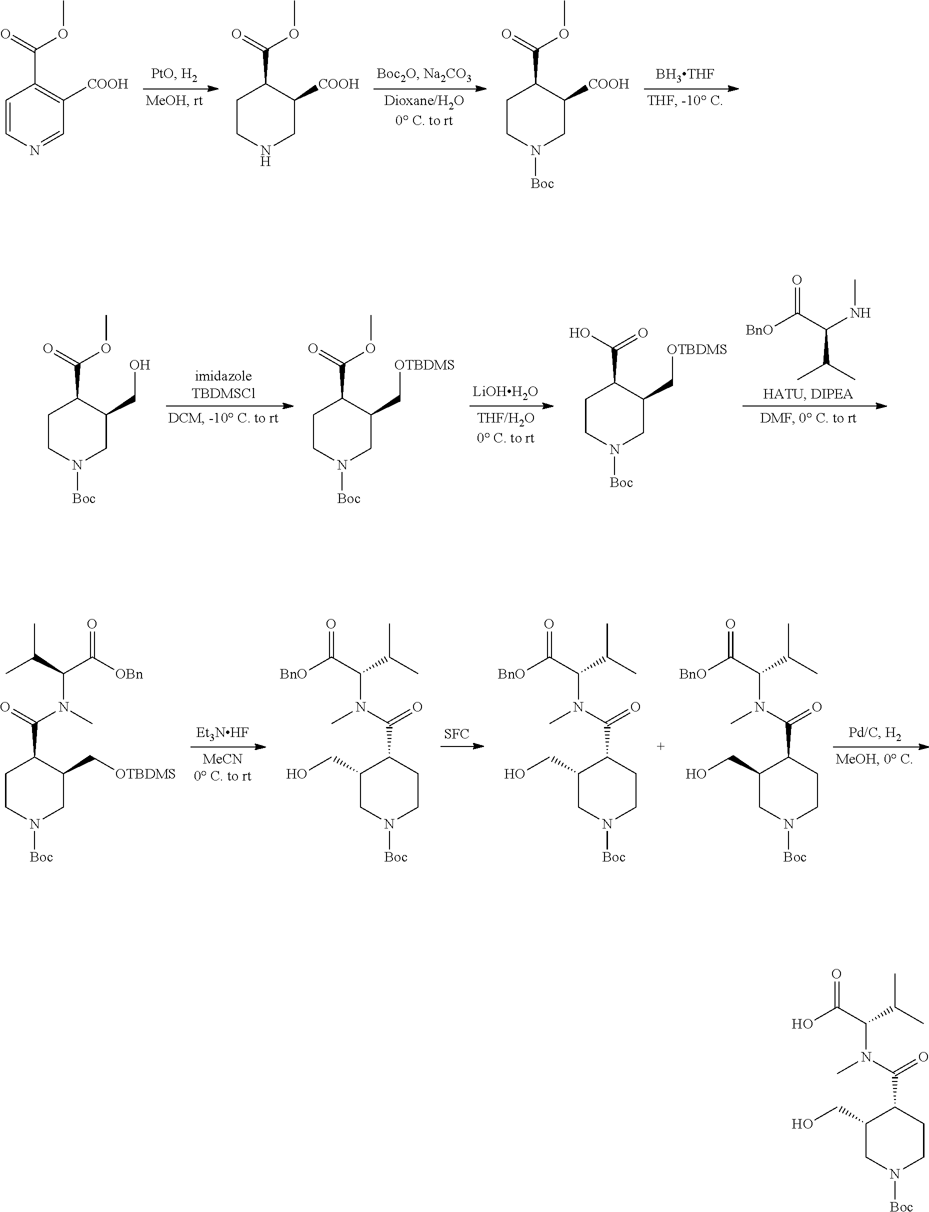

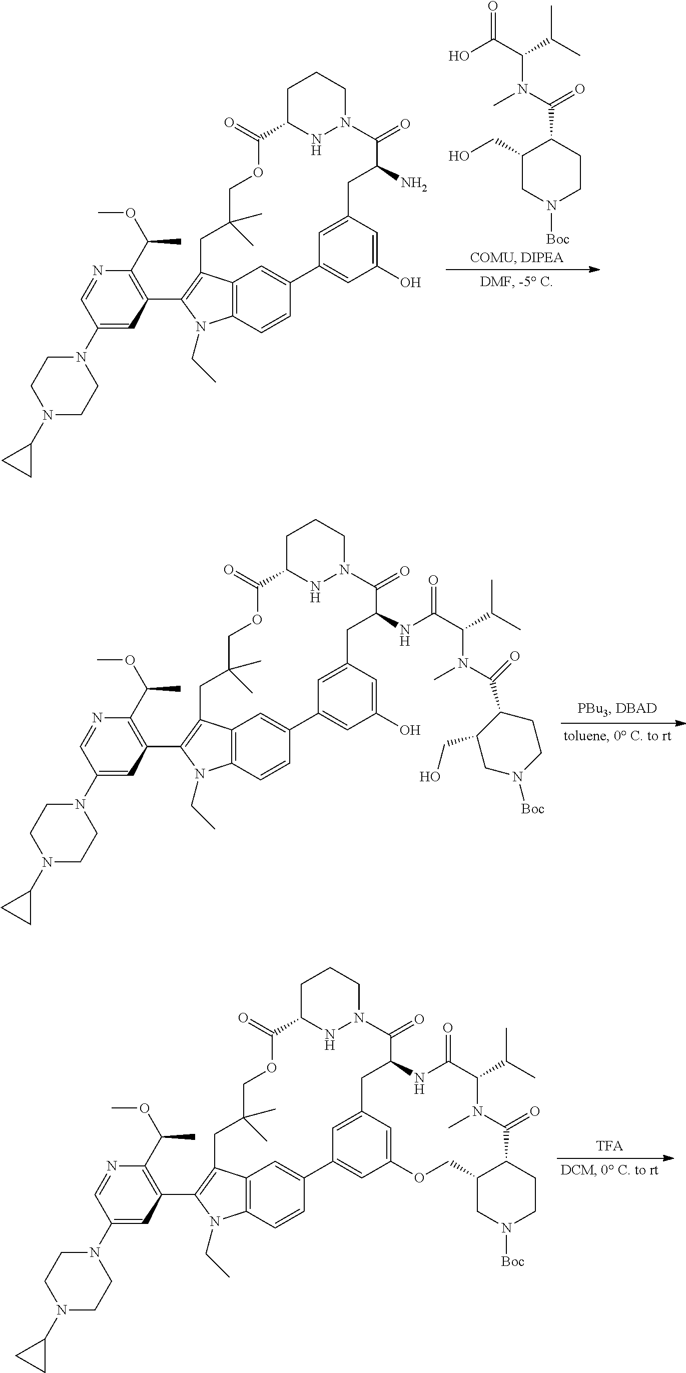

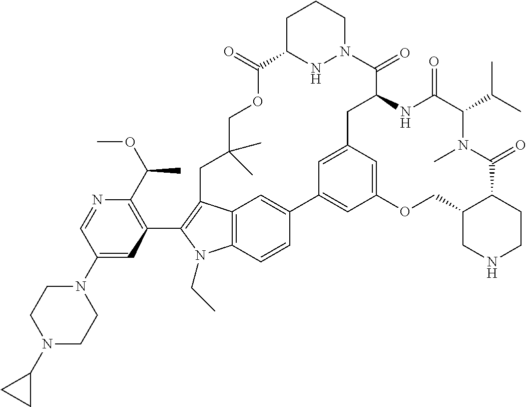

Step 1. To a stirred mixture of 4-(methoxycarbonyl)pyridine-3-carboxylic acid (50.0 g, 276 mmol) in MeOH (500 mL) added PtO₂ (5.00 g, 22.0 mmol) at room temperature. The reaction mixture was stirred for 16 h at 35° C. under an atmosphere of H₂. The reaction was filtered, and the filter pad was washed with MeOH (3×200 mL). The combined filtrate was concentrated under reduced pressure to afford (cis)-4-(methoxycarbonyl)piperidine-3-carboxylic acid (40 g, crude) as a brown oil. This material was taken directly to the next reaction without further purification. LCMS (ESI) m/z: [M+H] calcd for C₈H₁₃NO₄: 188.1; found 188.2.

Step 2. To a stirred solution of (cis)-4-(methoxycarbonyl)piperidine-3-carboxylic acid (40.0 g, crude) and Na₂CO₃ (67.0 g, 642 mmol) in H₂O (150 mL) and 1,4-dioxane (300 mL) was added (Boc)₂O (111 g, 535 mmol) in portions at 0° C. under an atmosphere of N₂. The reaction mixture was stirred for 1 h at room temperature, after which time the precipitated solids were collected by filtration, washed with H₂O (3×100 mL), washed with pet. ether (3×500 mL), acidified to pH 6 with aq. HCl, extracted with EtOAc (3×200 mL), and concentrated under reduced pressure to afford the crude (cis)-1-(tert-butoxycarbonyl)-4-(methoxycarbonyl)piperidine-3-carboxylic acid (56.0 g, crude) as a yellow oil. This material was taken directly to the next reaction without further purification. LCMS (ESI) m/z: [M−H] calcd for C₁₃H₂₁NO₆: 286.1; found 286.2.

Step 3. To a stirred mixture of (cis)-1-(tert-butoxycarbonyl)-4-(methoxycarbonyl)piperidine-3-carboxylic acid (25.7 g, crude) and THF (257 mL) was added borane·THF (1 M in THF, 179 mL, 179 mmol) over 10 min at −10° C. under an atmosphere of N₂. The resulting mixture was stirred for 1 h at −10° C. under an atmosphere of N₂. The reaction mixture was quenched by the addition of cold H₂O at 0° C. and was then extracted with EtOAc (4×300 mL). The combined organic extracts were then concentrated under reduced pressure to afford 1-(tert-butyl) 4-methyl (cis)-3-(hydroxymethyl)piperidine-1,4-dicarboxylate (14.7 g, crude) as a yellow oil. This material was taken directly to the next reaction without further purification. LCMS (ESI) m/z: [M+H] calcd for C₁₃H₂₃NO₅: 274.2; found 274.2.

Step 4. To a stirred solution of 1-(tert-butyl) 4-methyl (cis)-3-(hydroxymethyl)piperidine-1,4-dicarboxylate (14.7 g, crude) and DCM (147 mL) was added imidazole (9.15 g, 134 mmol) and tert-butyldimethylsilyl chloride (16.2 g, 108 mmol) in portions over 3 minutes at 0° C. The resulting mixture was stirred for 2 h at room temperature, after which time it was washed with H₂O (3×200 mL) and concentrated under reduced pressure. The residue was purified by normal phase flash column chromatography to afford 1-(tert-butyl) 4-methyl (cis)-3-(((tert-butyldimethylsilyl)oxy)methyl)piperidine-1,4-dicarboxylate (9 g, 13% yield over 4 steps) as a yellow oil. LCMS (ESI) m/z: [M+H—C₄H₈] calcd for C₁₉H₃₇NO₅Si: 332.2; found 332.2.

Step 5. To a stirred mixture of 1-(tert-butyl) 4-methyl (cis)-3-(((tert-butyldimethylsilyl)oxy)methyl)piperidine-1,4-dicarboxylate (8.8 g, 22.7 mmol) in THF (22 mL) was added a solution of LiOH·H₂O (1.9 g, 45.4 mmol) and H₂O (22 mL) dropwise at 0° C. under an atmosphere of N₂. The reaction mixture was stirred overnight at room temperature under an atmosphere of N₂, after which time it was acidified to pH 7 with sat. aq. HCl. The aqueous mixture was extracted with EtOAc (3×100 mL) and concentrated under reduced pressure to afford (cis)-1-(tert-butoxycarbonyl)-3-(((tert-butyldimethylsilyl)oxy)methyl)piperidine-4-carboxylic acid (6.20 g, crude) as a yellow oil. This material was taken directly to the next reaction without further purification. LCMS (ESI) m/z: [M+H] calcd for C₁₈H₃₅NO₅Si: 374.2; found 374.2.

Step 6. To a stirred mixture of benzyl methyl-L-valinate (7.10 g, 32.2 mmol) and DIPEA (14.0 mL, 80.4 mmol) in DMF (60 mL) were added (cis)-1-(tert-butoxycarbonyl)-3-(((tert-butyldimethylsilyl)oxy)methyl)piperidine-4-carboxylic acid (6.00 g, crude) and HATU (7.34 g, 19.3 mmol) in portions over 15 min at 0° C. The reaction mixture was stirred for 5 h at room temperature, after which time it was diluted with H₂O (30 mL), extracted with EtOAc (3×50 mL), washed with H₂O (3×50 mL), dried over anhydrous Na₂SO₄, filtered, and concentrated under reduced pressure. The residue was purified by normal phase prep-TLC to afford tert-butyl (cis)-4-(((S)-1-(benzyloxy)-3-methyl-1-oxobutan-2-yl)(methyl)carbamoyl)-3-(((tert-butyldimethylsilyl)oxy)methyl)piperidine-1-carboxylate (5.9 g, 47% yield over 2 steps) as a colorless oil. LCMS (ESI) m/z: [M+H] calcd for C₃₁H₅₂N₂O₆Si: 577.4; found 577.4.

Step 7. To a stirred solution of tert-butyl (cis)-4-(((S)-1-(benzyloxy)-3-methyl-1-oxobutan-2-yl)(methyl)carbamoyl)-3-(((tert-butyldimethylsilyl)oxy)methyl)piperidine-1-carboxylate (5.26 g, 9.12 mmol) in MeCN (5 mL) was added a solution of Et₃N·3HF (21 mL, 155 mmol) in MeCN (36 mL) dropwise over 10 minutes at 0° C. The reaction mixture was stirred for 4 h at room temperature, after which time it was basified to pH 7 with sat. aq. NaHCO₃, extracted with EtOAc (3×50 mL), and concentrated under reduced pressure. The residue was purified by normal phase flash column chromatography to afford tert-butyl (cis)-4-(((S)-1-(benzyloxy)-3-methyl-1-oxobutan-2-yl)(methyl)carbamoyl)-3-(hydroxymethyl)piperidine-1-carboxylate (3.0 g, 71%) as a yellow oil. LCMS (ESI) m/z: [M+H] calcd for C₂₅H₃₈N₂O₆: 463.3; found 463.3.

Step 8. The racemic tert-butyl (cis)-4-(((S)-1-(benzyloxy)-3-methyl-1-oxobutan-2-yl)(methyl)carbamoyl)-3-(hydroxymethyl)piperidine-1-carboxylate (3 g, 6.48 mmol) was purified by chiral prep-SFC to afford tert-butyl (3R,4R)-4-(((S)-1-(benzyloxy)-3-methyl-1-oxobutan-2-yl)(methyl)carbamoyl)-3-(hydroxymethyl)piperidine-1-carboxylate (1.49 g, assumed absolute configuration).

Step 9. To a stirred mixture of Pd/C (320 mg) and MeOH (3.2 mL) was added tert-butyl (3R,4R)-4-(((S)-1-(benzyloxy)-3-methyl-1-oxobutan-2-yl)(methyl)carbamoyl)-3-(hydroxymethyl)piperidine-1-carboxylate (320 mg, 0.692 mmol) at 0° C. The reaction mixture was stirred for 1 h at 0° C. under an atmosphere of H₂. The resulting mixture was filtered and then the filter cake was washed with MeOH (3×4 mL). The combined filtrate was concentrated under reduced pressure to afford N-((3R,4R)-1-(tert-butoxycarbonyl)-3-(hydroxymethyl)piperidine-4-carbonyl)-N-methyl-L-valine (300 mg, crude) as a clear oil. This material was taken directly to the next reaction without further purification. LCMS (ESI) m/z: [M+H] calcd for C₁₈H₃₂N₂O₆: 373.2; found 373.2.

Step 10. To a stirred mixture of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (522 mg, 0.683 mmol), N-((3R,4R)-1-(tert-butoxycarbonyl)-3-(hydroxymethyl)piperidine-4-carbonyl)-N-methyl-L-valine (280 mg, crude), DIPEA (1.77 g, 13.7 mmol) in DMF (5.3 mL) was added COMU (585 mg, 1.37 mmol) at −5° C. The reaction mixture was stirred overnight at −5° C., after which it was quenched with a mixture of ice and NaCl at 0° C., extracted with EtOAc (3×2 mL), washed with H₂O (2×5 mL), dried over anhydrous Na₂SO₄, filtered, and concentrated under reduced pressure. The residue was purified by normal phase prep-TLC to afford tert-butyl (3R,4R)-4-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-3-(hydroxymethyl)piperidine-1-carboxylate (260 mg, 31% yield over 2 steps) as a yellow solid. LCMS (ESI) m/z: [M+H] calcd for C₆₂H₈₇N₉O₁₀: 1118.7; found 1118.7.

Step 11. To a stirred mixture of di-tert-butyl azodicarboxylate (247 mg, 1.08 mmol) and tributylphosphane (217 mg, 1.08 mmol) in toluene (1.6 mL) was added a solution of tert-butyl (3R,4R)-4-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-3-(hydroxymethyl)piperidine-1-carboxylate (240 mg, 0.215 mmol) in toluene (0.8 mL) dropwise at 0° C. The reaction mixture was stirred overnight at room temperature, after which time it was concentrated under reduced pressure. The residue was purified by reversed-phase prep-HPLC to afford tert-butyl (4aR,7S,10S,16S,33aR)-22-(5-(4-cyclopropylpiperazin-1-yl)-2-((S)-1-methoxyethyl)pyridin-3-yl)-23-ethyl-7-isopropyl-6,20,20-trimethyl-5,8,11,17-tetraoxo-3,4,4a,5,6,7,8,9,10,11,14,15,16,17,19,20,21,23,33,33a-icosahydro-13H-12,16-epimino-24,26-etheno-10,29-methano-27,31-(metheno)pyrido[3,4-c]pyrrolo[3,4-v][1,18]dioxa[6,9,12]triazacyclotriacontine-2(1H)-carboxylate (90 mg, 37% yield) as a yellow solid. LCMS (ESI) m/z: [M+H] calcd for C₆₂H₈₅N₉O₉: 1100.7; found 1100.6.

Step 12. To a stirred solution of tert-butyl (4aR,7S,10S,16S,33aR)-22-(5-(4-cyclopropylpiperazin-1-yl)-2-((S)-1-methoxyethyl)pyridin-3-yl)-23-ethyl-7-isopropyl-6,20,20-trimethyl-5,8,11,17-tetraoxo-3,4,4a,5,6,7,8,9,10,11,14,15,16,17,19,20,21,23,33,33a-icosahydro-13H-12,16-epimino-24,26-etheno-10,29-methano-27,31-(metheno)pyrido[3,4-c]pyrrolo[3,4-v][1,18]dioxa[6,9,12]triazacyclotriacontine-2(1H)— carboxylate (90 mg, 0.082 mmol) in DCM (0.80 mL) was added TFA (0.3 mL, 3.92 mmol) dropwise at 0° C. The resulting mixture was stirred for 1 h at 0° C., after which time it was quenched with sat. aq. NaHCO₃ at 0° C., extracted with DCM (3×5 mL), dried over anhydrous Na₂SO₄, filtered, and concentrated under reduced pressure. The residue was purified by normal phase prep-TLC to afford (4aR,7S,10S,16S,33aR)-22-(5-(4-cyclopropylpiperazin-1-yl)-2-((S)-1-methoxyethyl)pyridin-3-yl)-23-ethyl-7-isopropyl-6,20,20-trimethyl-2,3,4,4a,6,7,9,10,15,16,19,20,21,23,33,33a-hexadecahydro-13H-12,16-epimino-24,26-etheno-10,29-methano-27,31-(metheno)pyrido[3,4-c]pyrrolo[3,4-v][1,18]dioxa[6,9,12]triazacyclotriacontine-5,8,11,17(1H,14H)-tetraone (9.9 mg, 13% yield) as a white solid. LCMS (ESI) m/z: [M+H] calcd for C₅₇H₇₇N₉O₇: 1000.6; found 1000.6; ¹H NMR (400 MHz, Acetonitrile-d₃) δ 8.40 (d, J=3.0 Hz, 1H), 8.07 (s, 1H), 7.68 (d, J=8.7 Hz, 1H), 7.49 (d, J=8.7 Hz, 1H), 7.36 (s, 1H), 7.20 (d, J=2.8 Hz, 2H), 7.10 (d, J=8.4 Hz, 1H), 6.81 (s, 1H), 5.27 (t, J=9.1 Hz, 1H), 4.85 (d, J=11.4 Hz, 1H), 4.62-4.36 (m, 4H), 4.27-4.09 (m, 4H), 3.80 (d, J=11.1 Hz, 1H), 3.70 (d, J=11.0 Hz, 1H), 3.29-3.24 (m, 1H), 3.21 (t, J=5.2 Hz, 5H), 3.17 (s, 3H), 3.13 (s, 1H), 3.05-2.92 (m, 3H), 2.88 (s, 3H), 2.84-2.75 (m, 3H), 2.72 (t, J=5.1 Hz, 6H), 2.69-2.57 (m, 2H), 2.12-2.09 (m, 2H), 1.89 (m, 1H), 1.84-1.54 (m, 6H), 1.36 (m, 7H), 1.14 (s, 3H), 1.01-0.81 (m, 10H), 0.78 (d, J=6.6 Hz, 3H), 0.51 (d, J=6.2 Hz, 3H), 0.44 (dd, J=6.2, 3.9 Hz, 2H), 0.38-0.32 (m, 2H).

### Synthesis of Compound A94—(6Z,9S,11aS,17S,31Z,31aS)-23-(5-(4-cyclopropylpiperazin-1-yl)-2-((S)-1-methoxyethyl)pyridin-3-yl)-24-ethyl-9-isopropyl-8,21,21-trimethyl-8,9,11a,14,15,16,17,20,21,22,24,31a-dodecahydro-2H,7H,12H,18H-3,6: 28,31-di(azeno)-13,17-epimino-25,27-etheno[1]oxa[10]thia[4,7]diazacyclotridecino[3,2-i]pyrrolo[3,4-q][1]oxa[12]thia[7]azacyclohenicosine-7,10,12,18(11H)-tetraone

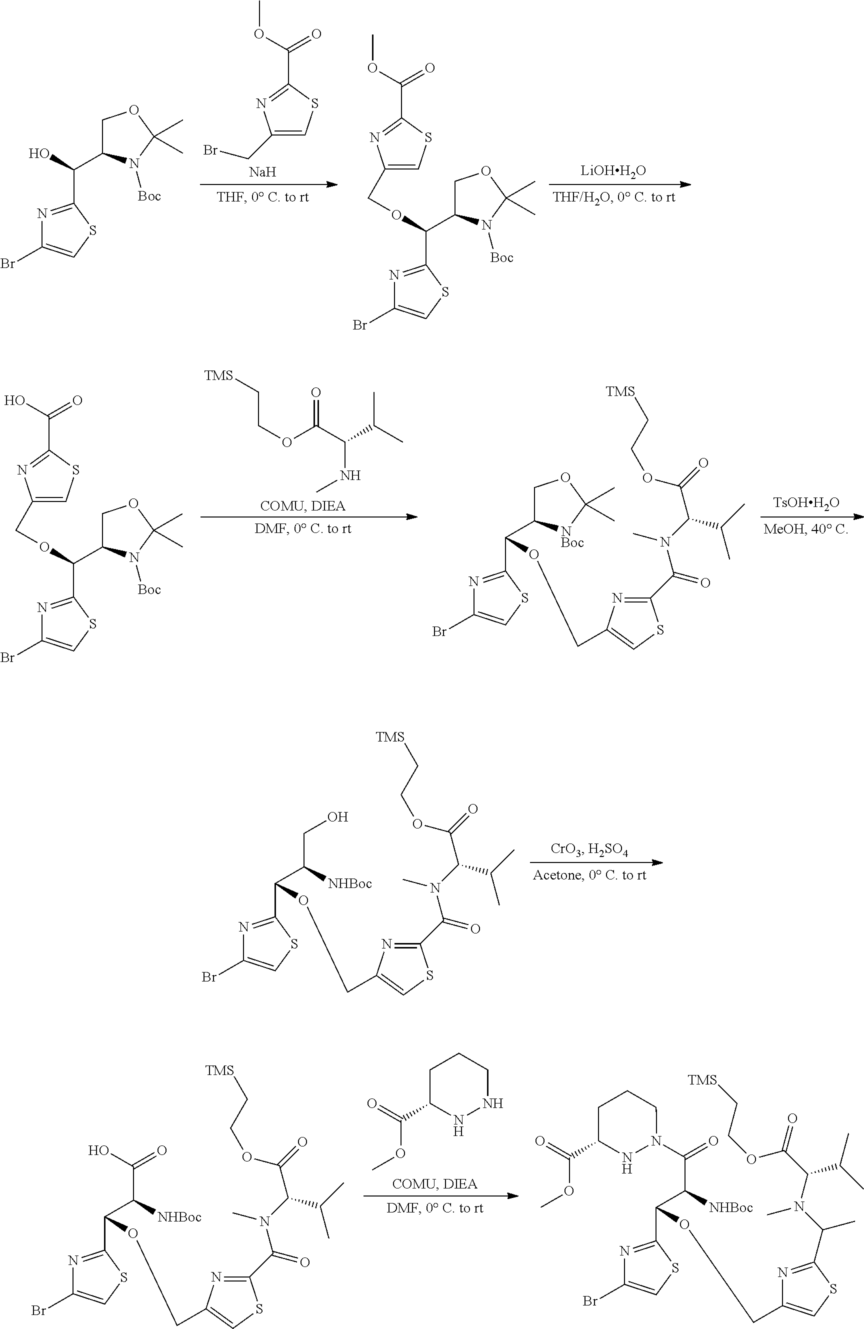

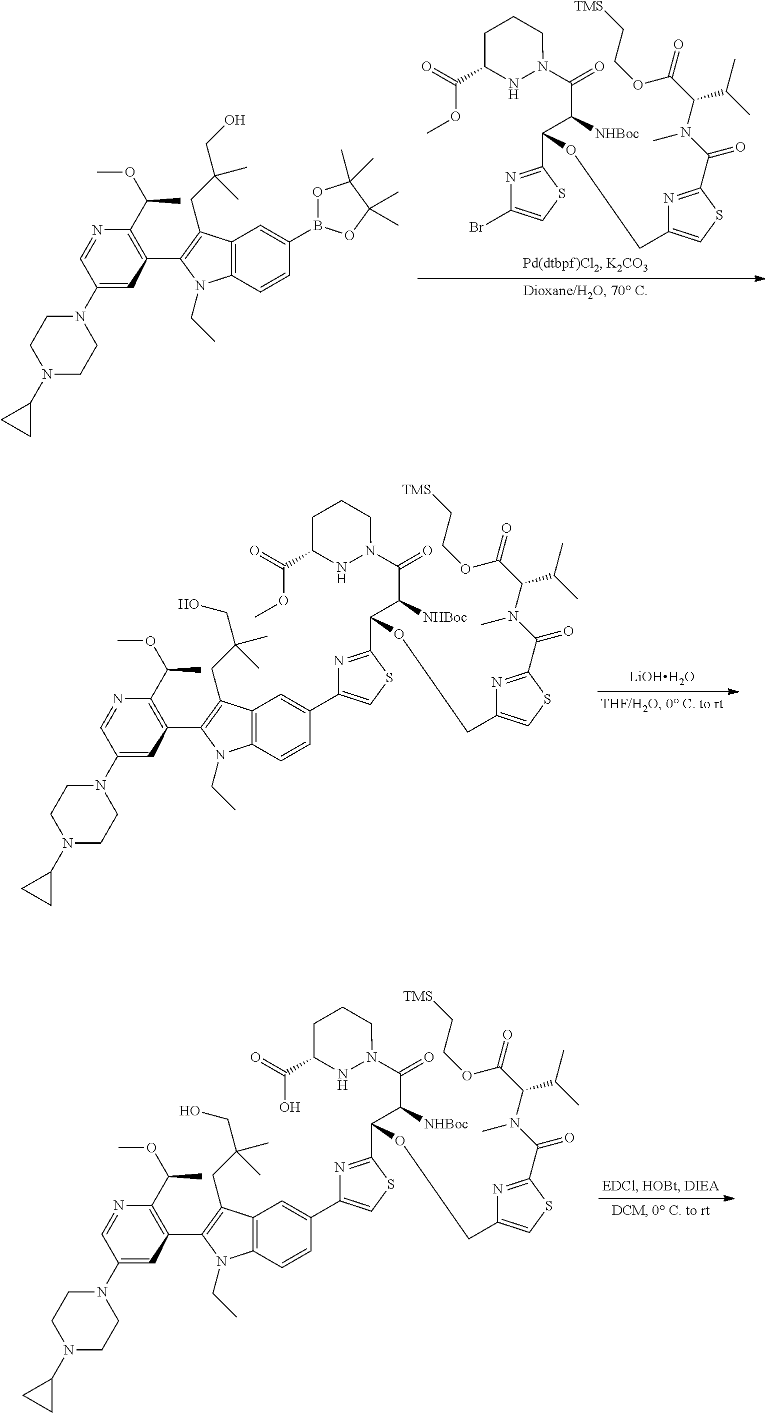

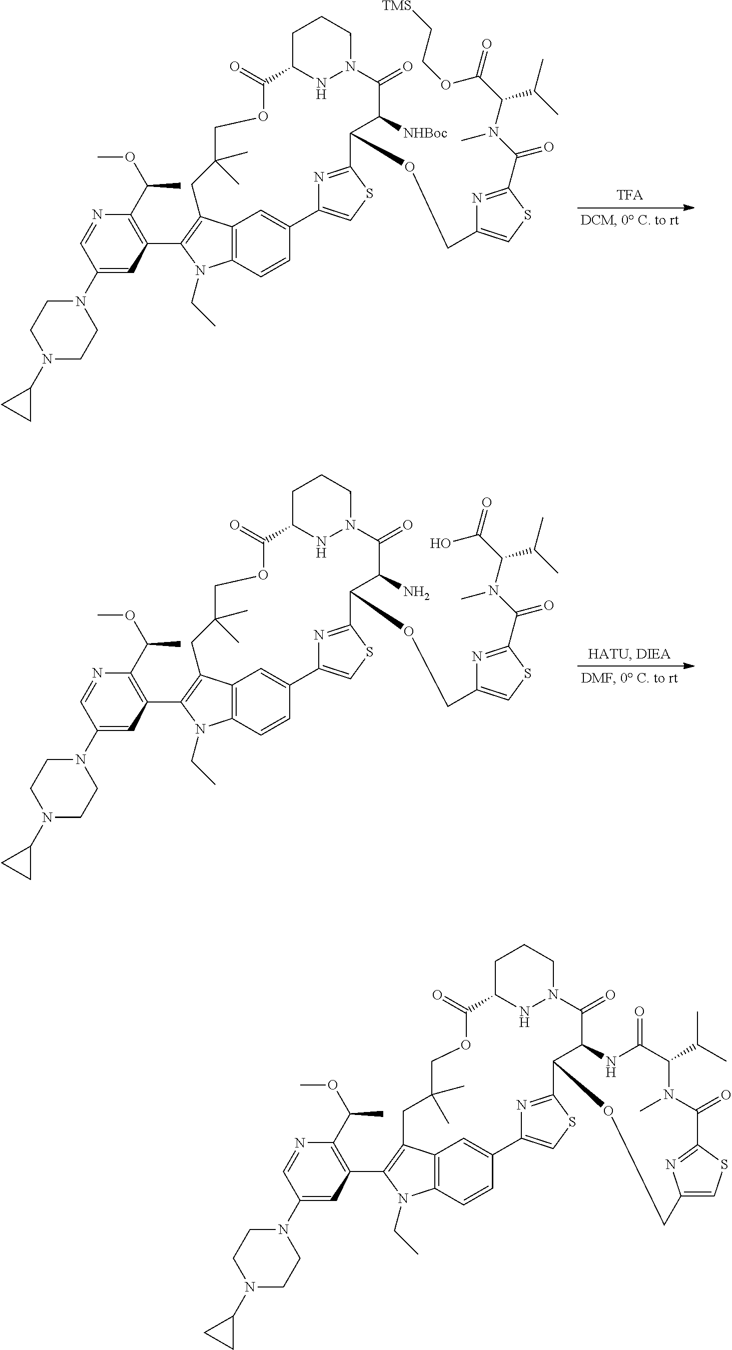

Step 1. To a stirred solution of tert-butyl (R)-4-((S)-(4-bromothiazol-2-yl)(hydroxy)methyl)-2,2-dimethyloxazolidine-3-carboxylate (5.00 g, 12.7 mmol) in THF (50 mL) was added NaH (1.53 g, 38.3 mmol, 60% dispersion in mineral oil) in portions at 0° C. The resulting mixture was stirred for 1 hour at 0° C., after which time a solution of methyl 4-(bromomethyl)thiazole-2-carboxylate (5.40 g, 22.9 mmol) in THF (10 mL) was added dropwise at 0° C. under an atmosphere of argon. The reaction mixture was stirred for 16 hours at room temperature, after which time it was quenched by the addition of ice water at 0° C., extracted with EtOAc (3×200 mL), dried over anhydrous Na₂SO₄, filtered, concentrated under reduced pressure, and purified by normal phase flash column chromatography to afford tert-butyl (R)-4-((S)-(4-bromothiazol-2-yl)((2-(methoxycarbonyl)thiazol-4-yl)methoxy)methyl)-2,2-dimethyloxazolidine-3-carboxylate (5.20 g, 75% yield) as a yellow oil. LCMS (ESI) m/z: [M+H] calcd for C₂₀H₂₆BrN₃O₆S₂: 548.0; found 548.0.

Step 2. To a stirred solution of tert-butyl (R)-4-((S)-(4-bromothiazol-2-yl)((2-(methoxycarbonyl)thiazol-4-yl)methoxy)methyl)-2,2-dimethyloxazolidine-3-carboxylate (5.20 g, 9.48 mmol) in THF (40 mL) and H₂O (10 mL) was added LiOH·H₂O (798 mg, 19.0 mmol) in portions at 0° C. The resulting mixture was stirred for 2 hours at room temperature under an atmosphere of argon. The reaction mixture was quenched by the addition of ice water and was then acidified to pH 5 by the addition of 1 M aqueous HCl solution, extracted with EtOAc (3×200 mL), dried over anhydrous Na₂SO₄, filtered, and concentrated under reduced pressure to give 4-(((S)-(4-bromothiazol-2-yl)((R)-3-(tert-butoxycarbonyl)-2,2-dimethyloxazolidin-4-yl)methoxy)methyl)thiazole-2-carboxylic acid (4.90 g, crude) as a yellow solid. This material was taken directly to the next reaction without further purification. LCMS (ESI) m/z: [M+H] calcd for C₁₉H₂₄BrN₃O₆S₂: 534.0; found 534.0.

Step 3. To a stirred mixture of 2-(trimethylsilyl)ethyl methyl-L-valinate (2.55 g, 11.0 mmol), DIEA (35.0 g, 271 mmol), and 4-(((S)-(4-bromothiazol-2-yl)((R)-3-(tert-butoxycarbonyl)-2,2-dimethyloxazolidin-4-yl)methoxy)methyl)thiazole-2-carboxylic acid (4.90 g, crude) in DMF (50 mL) was added COMU (4.30 g, 10.0 mmol) in portions at 0° C. The resulting mixture was stirred for 2 hours at room temperature under an atmosphere of argon. The reaction mixture was then quenched by the addition of sat. aq. NH₄Cl, extracted with EtOAc (200 mL), treated with brine (3×100 mL), dried over anhydrous Na₂SO₄, filtered, concentrated under reduced pressure, and purified by normal phase flash column chromatography to afford tert-butyl (R)-4-((S)-(4-bromothiazol-2-yl)((2-(methyl((S)-3-methyl-1-oxo-1-(2-(trimethylsilyl)ethoxy)butan-2-yl)carbamoyl)thiazol-4-yl)methoxy)methyl)-2,2-dimethyloxazolidine-3-carboxylate (5.10 g, 72% yield over 2 steps) as a red oil. LCMS (ESI) m/z: [M+H] calcd for C₃₀H₄₇BrN₄O₇S₂Si: 747.2; found 747.1.

Step 4. To a stirred solution of tert-butyl (R)-4-((S)-(4-bromothiazol-2-yl)((2-(methyl((S)-3-methyl-1-oxo-1-(2-(trimethylsilyl)ethoxy)butan-2-yl)carbamoyl)thiazol-4-yl)methoxy)methyl)-2,2-dimethyloxazolidine-3-carboxylate (5.10 g, 6.82 mmol) in MeOH (51 mL) added TsOH·H₂O (520 mg, 2.73 mmol) in portions at 0° C. The resulting mixture was stirred for 6 hours at 40° C. under an atmosphere of argon. The reaction mixture was concentrated under reduced pressure and purified by normal phase flash column chromatography to give 2-(trimethylsilyl)ethyl N-(4-(((1 S,2R)-1-(4-bromothiazol-2-yl)-2-((tert-butoxycarbonyl)amino)-3-hydroxypropoxy)methyl)thiazole-2-carbonyl)-N-methyl-L-valinate (2.50 g, 52% yield) as a yellow solid. LCMS (ESI) m/z: [M+H] calcd for C₂₇H₄₃BrN₄O₇S₂Si: 707.2; found 707.1.

Step 5. To a stirred solution of 2-(trimethylsilyl)ethyl N-(4-(((1S,2R)-1-(4-bromothiazol-2-yl)-2-((tert-butoxycarbonyl)amino)-3-hydroxypropoxy)methyl)thiazole-2-carbonyl)-N-methyl-L-valinate (2.90 g, 4.10 mmol) in acetone (30 mL) was added CrO₃ (4.1 mL, 8.2 mmol, 2.0 M in H₂SO₄) dropwise at 0° C. The resulting mixture was stirred for 1 hour at room temperature under an atmosphere of argon. The reaction mixture was then quenched by the addition of i-PrOH at 0° C., diluted with H₂O (100 mL), extracted with EtOAc (3×100 mL), dried over anhydrous Na₂SO₄, filtered, and concentrated under reduced pressure to give (2S,3S)-3-(4-bromothiazol-2-yl)-2-((tert-butoxycarbonyl)amino)-3-((2-(methyl((S)-3-methyl-1-oxo-1-(2-(trimethylsilyl)ethoxy)butan-2-yl)carbamoyl)thiazol-4-yl)methoxy)propanoic acid (3.00 g, crude) as an off-white solid. This material was taken directly to the next reaction without further purification. LCMS (ESI) m/z: [M+H] calcd for C₂₇H₄₁BrN₄O₈S₂Si: 721.1; found 721.1.

Step 6. To a stirred mixture of methyl (S)-hexahydropyridazine-3-carboxylate dihydrochloride (1.08 g, 5.20 mmol), DIEA (10.8 g, 83.2 mmol), and (2S,3S)-3-(4-bromothiazol-2-yl)-2-((tert-butoxycarbonyl)amino)-3-((2-(methyl((S)-3-methyl-1-oxo-1-(2-(trimethylsilyl)ethoxy)butan-2-yl)carbamoyl)thiazol-4-yl)methoxy)propanoic acid (3.00 g, crude) in DMF (30 mL) was added COMU (1.78 mg, 0.416 μmol) in portions at 0° C. The resulting mixture was stirred for 1 hour at room temperature under an atmosphere of argon. The reaction mixture was then quenched by the addition of sat. aq. NH₄Cl 0° C., extracted with EtOAc (100 mL), treated with brine (3×50 mL), dried over anhydrous Na₂SO₄, filtered, concentrated under reduced pressure and purified by normal phase flash column chromatography to give methyl (S)-1-((2S,3S)-3-(4-bromothiazol-2-yl)-2-((tert-butoxycarbonyl)amino)-3-((2-(methyl((S)-3-methyl-1-oxo-1-(2-(trimethylsilyl)ethoxy)butan-2-yl)carbamoyl)thiazol-4-yl)methoxy)propanoyl)hexahydropyridazine-3-carboxylate (2.40 g, 69% yield over 2 steps) as a light yellow solid. LCMS (ESI) m/z: [M+H] calcd for C₃₃H₅₁BrN₆O₉S₂Si: 847.2; found 847.5.

Step 7. To a stirred mixture of (S)-3-(2-(5-(4-cyclopropylpiperazin-1-yl)-2-(1-methoxyethyl)pyridin-3-yl)-1-ethyl-5-(4,4,5,5-tetramethyl-1,3,2-dioxaborolan-2-yl)-1H-indol-3-yl)-2,2-dimethylpropan-1-ol (1.10 g, 1.78 mmol), methyl (S)-1-((2S,3S)-3-(4-bromothiazol-2-yl)-2-((tert-butoxycarbonyl)amino)-3-((2-(methyl((S)-3-methyl-1-oxo-1-(2-(trimethylsilyl)ethoxy)butan-2-yl)carbamoyl)thiazol-4-yl)methoxy)propanoyl)hexahydropyridazine-3-carboxylate (1.50 g, 1.77 mmol), and K₂CO₃ (612 mg, 4.43 mmol) in 1,4-dioxane (10 mL) and H₂O (2 mL) was added Pd(dtbpf)Cl₂ (233 mg, 0.357 mmol) in portions at 0° C. The resulting mixture was stirred for 3 hours at 70° C. under an atmosphere of argon. The reaction mixture was quenched by the addition of ice water, extracted with EtOAc (3×50 mL), dried over anhydrous Na₂SO₄, filtered, concentrated under reduced pressure, and purified by normal phase flash column chromatography to give methyl (S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-3-((2-(methyl((S)-3-methyl-1-oxo-1-(2-(trimethylsilyl)ethoxy)butan-2-yl)carbamoyl)thiazol-4-yl)methoxy)propanoyl)hexahydropyridazine-3-carboxylate (1.40 g, 62% yield) as a brown solid. LCMS (ESI) m/z: [M+H] calcd for C₆₃H₉₂N₁₀O₁₁S₂Si: 1257.6; found 1257.6.

Step 8. To a stirred solution of methyl (S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-3-((2-(methyl((S)-3-methyl-1-oxo-1-(2-(trimethylsilyl)ethoxy)butan-2-yl)carbamoyl)thiazol-4-yl)methoxy)propanoyl)hexahydropyridazine-3-carboxylate (1.40 g, 1.11 mmol) in THF (12 mL) and H₂O (3 mL) was added LiOH·H₂O (112 mg, 2.67 mmol) in portions at 0° C. The resulting mixture was stirred for 2 hours at room temperature under an atmosphere of argon. The reaction mixture was then quenched by the addition of ice water, acidified to pH 5 by the addition of 1 M aq. HCl, extracted with EtOAc (3×50 mL), dried over anhydrous Na₂SO₄, filtered, and concentrated under reduced pressure to give (S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-3-((2-(methyl((S)-3-methyl-1-oxo-1-(2-(trimethylsilyl)ethoxy)butan-2-yl)carbamoyl)thiazol-4-yl)methoxy)propanoyl)hexahydropyridazine-3-carboxylic acid (1.40 g, crude) as a brown solid. This material was taken directly to the next reaction without further purification. LCMS (ESI) m/z: [M+H] calcd for C₆₂H₉₀N₁₀O₁₁S₂Si: 1243.6; found 1243.7.

Step 9. To a stirred mixture of (S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-3-((2-(methyl((S)-3-methyl-1-oxo-1-(2-(trimethylsilyl)ethoxy)butan-2-yl)carbamoyl)thiazol-4-yl)methoxy)propanoyl)hexahydropyridazine-3-carboxylic acid (1.40 g, crude), DIEA (5.83 g, 45.1 mmol) and HOBt (760 mg, 5.62 mmol) in DCM (140 mL) was added EDCI (6.49 g, 33.9 mmol) in portions at 0° C. The resulting mixture was stirred for 16 hours at room temperature under an atmosphere of argon. The reaction mixture was then quenched by the addition of ice water, extracted with EtOAc (300 mL), treated with brine (3×100 mL), dried over anhydrous Na₂SO₄, filtered, concentrated under reduced pressure, and purified by normal phase prep-TLC afford 2-(trimethylsilyl)ethyl N-(4-((((63S,3S,4S,Z)-4-((tert-butoxycarbonyl)amino)-12-(5-(4-cyclopropylpiperazin-1-yl)-2-((S)-1-methoxyethyl)pyridin-3-yl)-11-ethyl-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane-3-yl)oxy)methyl)thiazole-2-carbonyl)-N-methyl-L-valinate (500 mg, 37% yield over 2 steps) as a white solid. LCMS (ESI) m/z: [M+H] calcd for C₆₂H₈₈N₁₀O₁₀S₂Si: 1225.6; found 1225.7.

Step 10. To a stirred solution of 2-(trimethylsilyl)ethyl N-(4-((((6³S,3S,4S,Z)-4-((tert-butoxycarbonyl)amino)-12-(5-(4-cyclopropylpiperazin-1-yl)-2-((S)-1-methoxyethyl)pyridin-3-yl)-11-ethyl-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane- 3-yl)oxy)methyl)thiazole-2-carbonyl)-N-methyl-L-valinate (300 mg, 0.245 mmol) in DCM (6 mL) was added TFA (3.00 mL, 40.4 mmol) dropwise at 0° C. under an atmosphere of argon. The resulting mixture was stirred for 2 hours at room temperature and was then concentrated under reduced pressure to yield N-(4-((((63S,3S,4S,Z)-4-amino-12-(5-(4-cyclopropylpiperazin-1-yl)-2-((S)-1-methoxyethyl)pyridin-3-yl)-11-ethyl-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane-3-yl)oxy)methyl)thiazole-2-carbonyl)-N-methyl-L-valine (500 mg, crude) as a yellow solid. This material was taken directly to the next reaction without further purification. LCMS (ESI) m/z: [M+H] calcd for C₅₂H₆₈N₁₀O₈S₂: 1025.5; found 1025.5.

Step 11. To a stirred mixture of N-(4-((((63S,3S,4S,Z)-4-amino-12-(5-(4-cyclopropylpiperazin-1-yl)-2-((S)-1-methoxyethyl)pyridin-3-yl)-11-ethyl-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane-3-yl)oxy)methyl)thiazole-2-carbonyl)-N-methyl-L-valine (700 mg, crude) and DIEA (1.76 g, 13.6 mmol) in DMF (350 mL) was added HATU (520 mg, 1.37 mmol) in portions at 0° C. The resulting mixture was stirred for 2 hours at room temperature under an atmosphere of argon. The reaction mixture was then quenched by the addition of ice water, extracted with EtOAc (300 mL), treated with brine (3×300 mL), dried over anhydrous Na₂SO₄, filtered, and concentrated under reduced pressure. The residue was purified by normal phase prep-TLC and was subsequently purified by reversed phase prep-HPLC to give (6Z,9S,11aS,17S,31Z,31aS)-23-(5-(4-cyclopropylpiperazin-1-yl)-2-((S)-1-methoxyethyl)pyridin-3-yl)-24-ethyl-9-isopropyl-8,21,21-trimethyl-8,9,11a,14,15,16,17,20,21,22,24,31a-dodecahydro-2H,7H,12H,18H-3,6:28,31-di(azeno)-13,17-epimino-25,27-etheno[1]oxa[10]thia[4,7]diazacyclotridecino[3,2-]pyrrolo[3,4-q][1]oxa[12]thia[7]azacyclohenicosine-7,10,12,18(11H)-tetraone (11.0 mg, 2% yield over 2 steps) as a white solid. LCMS (ESI) m/z: [M+H] calcd for C₅₂H₆₆N₁₀O₇S₂: 1007.5; found 1007.4; ¹H NMR (400 MHz, DMSO-d₆) δ 8.56-8.44 (m, 1H), 8.39 (s, 1H), 7.95 (s, 1H), 7.90-7.72 (m, 2H), 7.71-7.60 (m, 1H), 7.59-7.48 (m, 1H), 7.39-7.17 (m, 1H), 6.11 (br s, 1H), 5.37-5.26 (m, 1H), 5.14-4.93 (m, 2H), 4.76-4.69 (m, 1H), 4.25-4.03 (m, 3H), 4.01-3.79 (m, 3H), 3.62 (d, J=10.3 Hz, 1H), 3.50 (d, J=10.5 Hz, 1H), 3.22 (s, 4H), 3.17 (s, 3H), 2.88 (s, 3H), 2.77 (s, 1H), 2.68 (p, J=4.4 Hz, 4H), 2.29-2.10 (m, 1H), 2.06-1.98 (m, 1H), 1.79 (t, J=14.2 Hz, 1H), 1.66 (s, 1H), 1.55-1.07 (m, 9H), 1.04-0.79 (m, 7H), 0.62 (s, 2H), 0.54 (s, 2H), 0.43 (dd, J=6.2, 3.8 Hz, 3H), 0.35 (q, J=3.3 Hz, 2H).

### Synthesis of Compound A16—(3⁵S,3⁸S,3⁹S,3^{17a}R,5³S,Z)-1²-(5-(4-cyclopropylpiperazin-1-yl)-2-((S)-1-methoxyethyl)pyridin-3-yl)-1¹-ethyl-3⁵-isopropyl-3¹⁰,9,9-trimethyl-3²,3³,3⁶,3⁷,3⁸,3⁹,3¹⁰,3¹¹, 3¹⁷,3^{17a},5¹,5²,5³,5⁴,5⁵,5⁶-hexadecahydro-1¹H,3¹H,3⁵H-7-oxa-3(9,8)-benzo[I]pyrrolo[2,1-c][1]oxa[4,7,10]triazacyclotridecina-2(4,2)-thiazola-1(5,3)-indola-5(1,3)-pyridazinacyclodecaphane-3⁶,4,6-trione

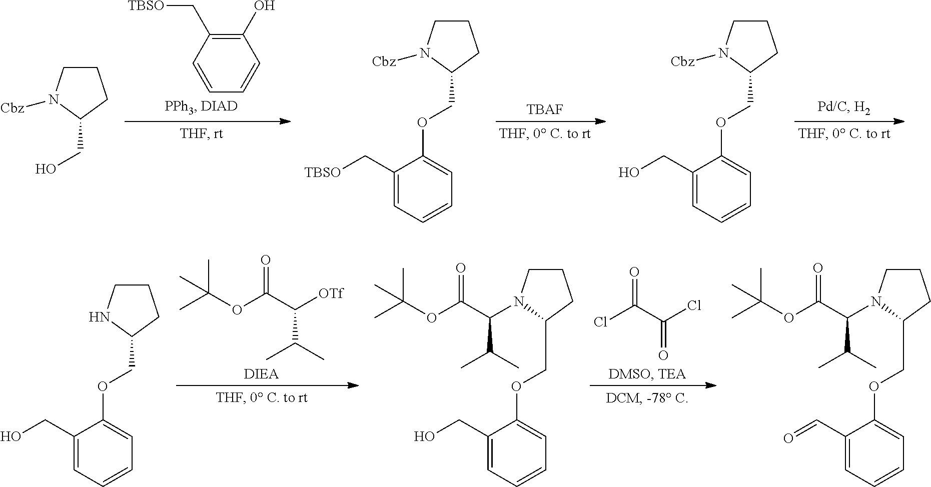

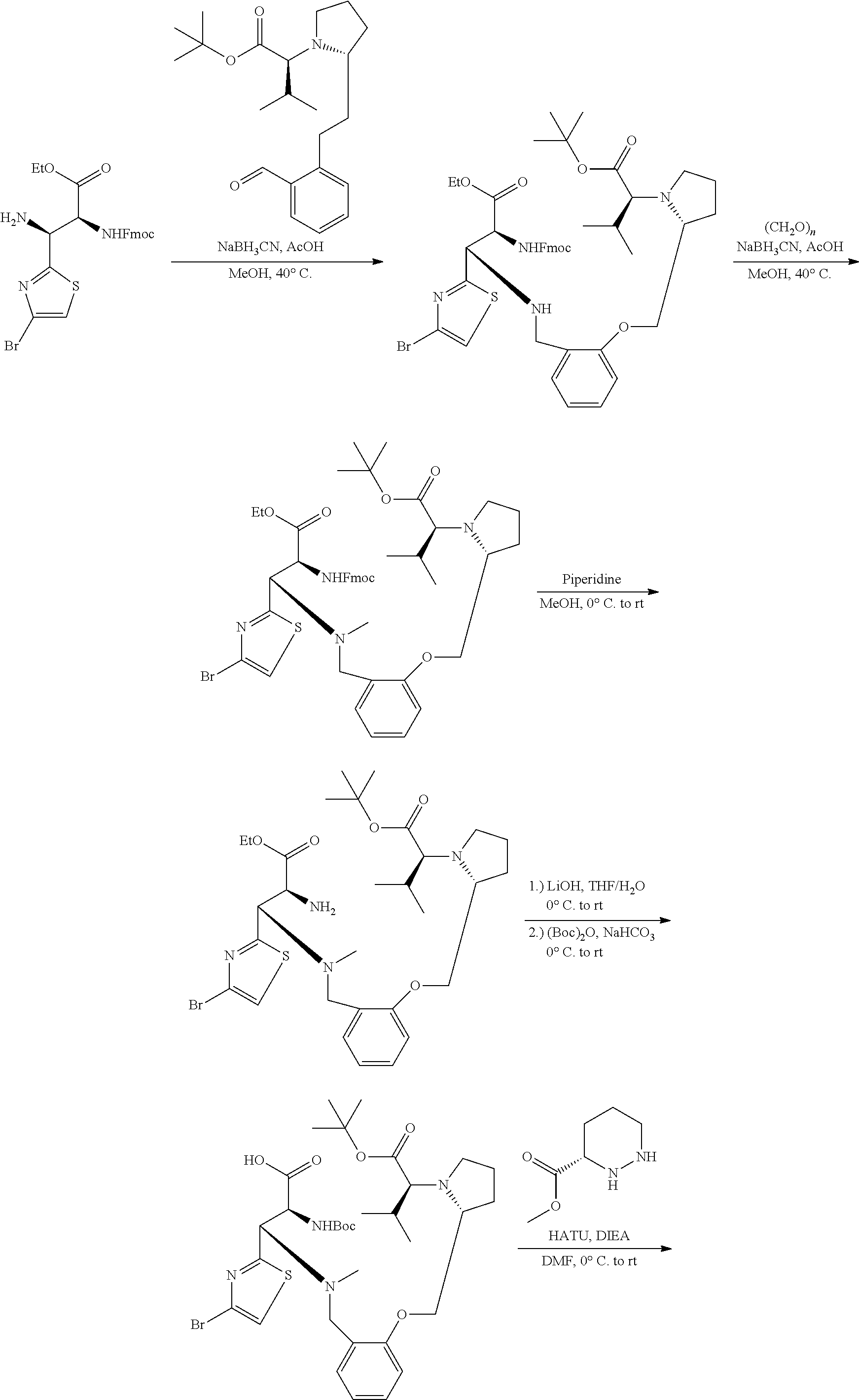

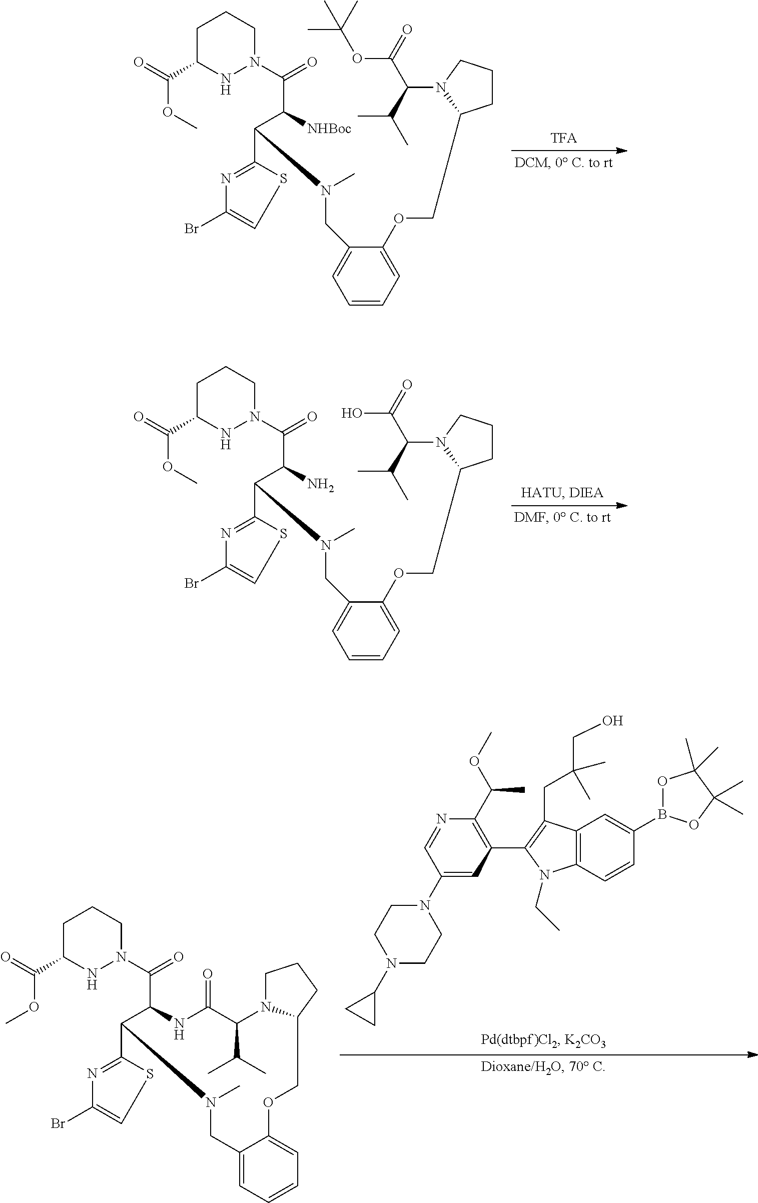

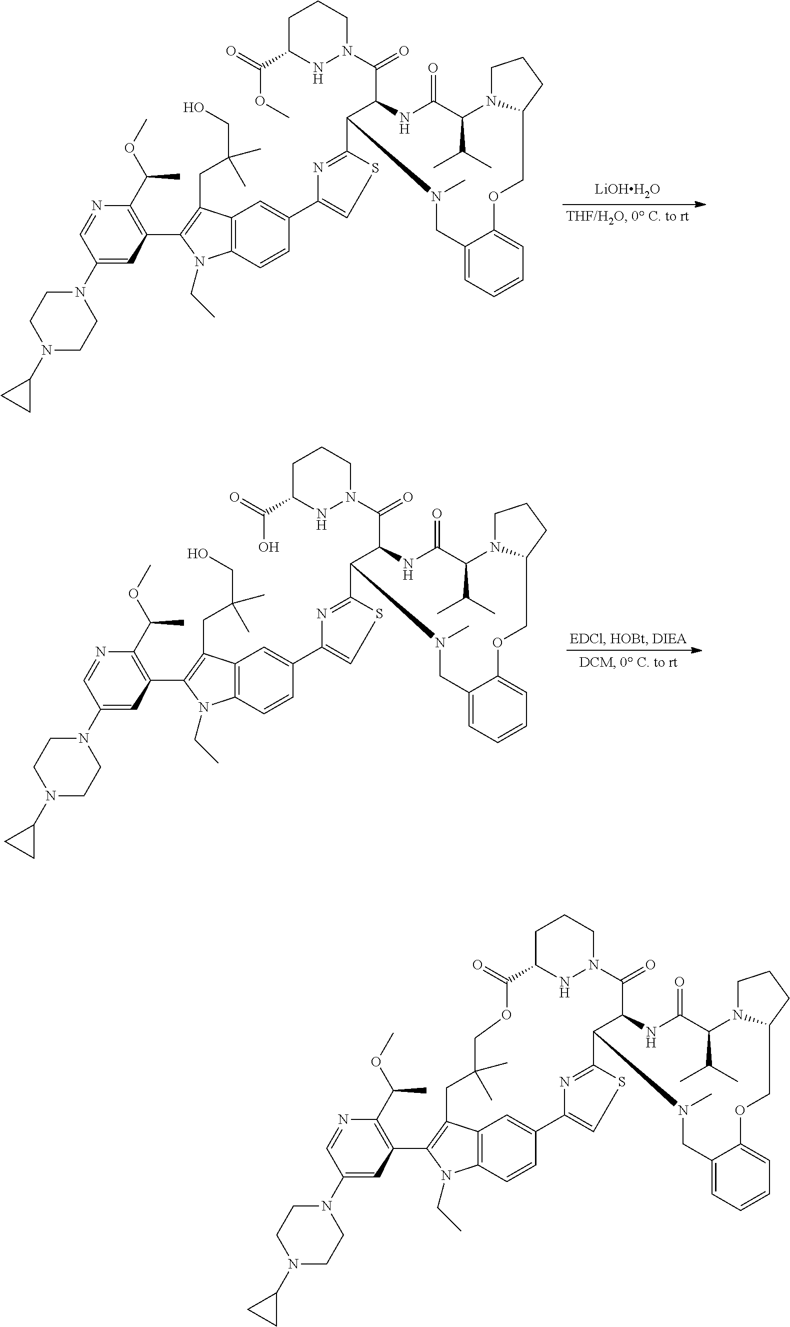

Step 1. Into a stirred solution of benzyl (R)-2-(hydroxymethyl)pyrrolidine-1-carboxylate (25.0 g, 106 mmol) and 2-(((tert-butyldimethylsilyl)oxy)methyl)phenol (38.0 g, 159 mmol) in THF (250 mL) were added PPh₃ (69.7 g, 266 mmol) and DIAD (43.0 g, 213 mmol) in portions at 0 NC. The resulting mixture was stirred for 16 hours at room temperature under an atmosphere of argon. The reaction mixture was quenched by the addition of cold H₂O, extracted with EtOAc (3×1 L), dried over anhydrous Na₂SO₄, filtered, concentrated under reduced pressure, and purified by normal phase flash column chromatography to give benzyl (R)-2-((2-(((tert-butyldimethylsilyl)oxy)methyl)phenoxy)methyl)pyrrolidine-1-carboxylate (7.70 g, 13% yield) as a yellow oil. LCMS (ESI) m/z: [M+H] calcd for C₂₆H₃₇NO₄Si: 456.3; found 456.1.

Step 2. Into a stirred 1.0 M solution of TBAF in THF (80 mL, 80 mmol) was added benzyl (R)-2-((2-(((tert-butyldimethylsilyl)oxy)methyl)phenoxy)methyl)pyrrolidine-1-carboxylate (7.70 g, 16.9 mmol) at 0° C. The resulting mixture was stirred for 2 hours at room temperature under an atmosphere of argon. The reaction mixture was then quenched by the addition of sat. aq. NH₄Cl at 0° C., extracted with EtOAc (3×100 mL), dried over anhydrous Na₂SO₄, filtered, concentrated under reduced pressure, and purified by normal phase flash column chromatography to yield benzyl (R)-2-((2-(hydroxymethyl)phenoxy)methyl)pyrrolidine-1-carboxylate (4.10 g, 64% yield) as a yellow oil. LCMS (ESI) m/z: [M+H] calcd for C₂₀H₂₃NO₄: 342.2; found 342.0.

Step 3. To a stirred solution of benzyl (R)-2-((2-(hydroxymethyl)phenoxy)methyl)pyrrolidine-1-carboxylate (7.80 g, 22.8 mmol) in THF (80 mL) was added 10% Pd/C (4.00 g) in portions at 0° C. The resulting mixture was stirred for 16 hours at room temperature under an atmosphere of H₂. The reaction mixture was then filtered through Celite®, the filter cake washed with EtOAc (3×100 mL), and the filtrate concentrated under reduced pressure to give (R)-(2-(pyrrolidin-2-ylmethoxy)phenyl)methanol (4.40 g, crude) as a yellow oil. This material was taken directly to the next reaction without further purification. LCMS (ESI) m/z: [M+H] calcd for C₁₂H₁₇NO₂: 208.1; found 208.1.

Step 4. To a stirred solution of (R)-(2-(pyrrolidin-2-ylmethoxy)phenyl)methanol (4.40 g, crude) and DIEA (13.7 g, 106 mmol) in THF (44 mL) was added tert-butyl (R)-3-methyl-2-(((trifluoromethyl)sulfonyl)oxy)butanoate (19.5 g, 63.7 mmol) dropwise at 0° C. The resulting mixture was stirred for 16 hours at room temperature under an atmosphere of argon. The reaction mixture was then quenched by the addition of sat. aq. NH₄Cl at 0° C., extracted with EtOAc (3×100 mL), dried over anhydrous Na₂SO₄, filtered, concentrated under reduced pressure, and purified by normal phase prep-TLC to give tert-butyl (S)-2-((R)-2-((2-(hydroxymethyl)phenoxy)methyl)pyrrolidin-1-yl)-3-methylbutanoate (4.90 g, 59% yield over 2 steps) as a yellow oil. LCMS (ESI) m/z: [M+H] calcd for C₂₁H₃₃NO₄: 364.2; found 364.2.

Step 5. To a stirred solution of oxalyl chloride (3.29 g, 25.9 mmol) in DCM (33 mL) was added a solution of DMSO (3.03 g, 38.8 mmol) in DCM (30 mL) dropwise over 10 minutes at −78° C. This mixture was stirred for an additional 30 minutes at −78° C., after which time a solution of tert-butyl (S)-2-((R)-2-((2-(hydroxymethyl)phenoxy)methyl)pyrrolidin-1-yl)-3-methylbutanoate (4.70 g, 12.9 mmol) in DCM (47 mL) was added dropwise over 10 minutes at 0° C. This mixture was stirred for an additional 30 minutes at −78° C., after which time a solution of TEA (7.85 g, 77.6 mmol) in DCM (40 mL) was added dropwise over 10 minutes at −78° C. The resulting mixture was stirred for 1 hour at −78° C. The reaction mixture was quenched by the addition of sat. aq. NH₄Cl at 0° C., extracted with DCM (3×200 mL), and concentrated under reduced pressure to yield tert-butyl (S)-2-((R)-2-((2-formylphenoxy)methyl)pyrrolidin-1-yl)-3-methylbutanoate (5.00 g, crude) as a clear oil. This material was taken directly to the next reaction without further purification. LCMS (ESI) m/z: [M+H] calcd for C₂₁H₃₁NO₄: 362.2; found 362.2.

Step 6. To a stirred solution of ethyl (2S,3S)-2-((((9H-fluoren-9-yl)methoxy)carbonyl)amino)-3-amino-3-(4-bromothiazol-2-yl)propanoate (5.79 g, 11.2 mmol) and tert-butyl (S)-2-((R)-2-((2-formylphenoxy)methyl)pyrrolidin-1-yl)-3-methylbutanoate (5.70 g, crude) in MeOH (57 mL) were added acetic acid (1.89 g, 31.5 mmol) and NaBH₃CN (3.3 g, 52.5 mmol) in portions at 0° C. The resulting mixture was stirred for 16 hours at 40° C. under an atmosphere of argon. The reaction mixture was quenched by the addition of sat. aq. NaHCO₃ at 0° C., extracted with EtOAc (3×100 mL), dried over anhydrous Na₂SO₄, filtered, concentrated under reduced pressure, and purified by normal phase flash column chromatography to give tert-butyl (S)-2-((R)-2-((2-((((1 S,2S)-2-((((9H-fluoren-9-yl)methoxy)carbonyl)amino)-1-(4-bromothiazol-2-yl)-3-ethoxy-3-oxopropyl)amino)methyl)phenoxy)methyl)pyrrolidin-1-yl)-3-methylbutanoate (6.00 g, 56% yield) as a white solid. LCMS (ESI) m/z: [M+H] calcd for C₄₄H₅₃BrN₄O₇S: 861.3; found 861.2.

Step 7. To a stirred solution of tert-butyl (S)-2-((R)-2-((2-((((1 S,2S)-2-((((9H-fluoren-9-yl)methoxy)carbonyl)amino)-1-(4-bromothiazol-2-yl)-3-ethoxy-3-oxopropyl)amino)methyl)phenoxy)methyl)pyrrolidin-1-yl)-3-methylbutanoate (6.50 g, 7.54 mmol) and paraformaldehyde (2.04 g, 22.6 mmol) in MeOH (65 mL) were added acetic acid (1.36 g, 22.6 mmol) and NaBH₃CN (2.38 g, 37.9 mmol) in portions at 0° C. The resulting mixture was stirred for 16 hours at 40° C. under an atmosphere of argon. The reaction mixture was then quenched by the addition of sat. aq. NaHCO₃ at 0° C., extracted with EtOAc (3×100 mL), dried over anhydrous Na₂SO₄, filtered, concentrated under reduced pressure, and purified by normal phase flash column chromatography to give tert-butyl (S)-2-((R)-2-((2-((((1S,2S)-2-((((9H-fluoren-9-yl)methoxy)carbonyl)amino)-1-(4-bromothiazol-2-yl)-3-ethoxy-3-oxopropyl)(methyl)amino)methyl)phenoxy)methyl)pyrrolidin-1-yl)-3-methylbutanoate (6.00 g, 82% yield) as a white solid. LCMS (ESI) m/z: [M+H] calcd for C₄₅H₅₅BrN₄O₇S: 875.3; found 875.2.

Step 8. To a stirred solution of tert-butyl (S)-2-((R)-2-((2-((((1 S,2S)-2-((((9H-fluoren-9-yl)methoxy)carbonyl)amino)-1-(4-bromothiazol-2-yl)-3-ethoxy-3-oxopropyl)(methyl)amino)methyl)phenoxy)methyl)pyrrolidin-1-yl)-3-methylbutanoate (5.50 g, 6.28 mmol) in MeCN (55 mL) was added piperidine (11 mL) dropwise over 5 minutes at 0° C. The resulting mixture was stirred for 2 hours at room temperature under an atmosphere of argon. The reaction mixture was then concentrated under reduced pressure and purified by normal phase flash column chromatography to give tert-butyl (S)-2-((R)-2-((2-((((1S,2S)-2-amino-1-(4-bromothiazol-2-yl)-3-ethoxy-3-oxopropyl)(methyl)amino)methyl)phenoxy)methyl)pyrrolidin-1-yl)-3-methylbutanoate (4.20 g, 92% yield) as a yellow oil. LCMS (ESI) m/z: [M+H] calcd for C₃₀H₄₅BrN₄O₅S: 653.2; found 653.1.

Step 9. To a stirred solution of tert-butyl (S)-2-((R)-2-((2-((((1 S,2S)-2-amino-1-(4-bromothiazol-2-yl)-3-ethoxy-3-oxopropyl)(methyl)amino)methyl)phenoxy)methyl)pyrrolidin-1-yl)-3-methylbutanoate (4.20 g, 6.43 mmol) in THF (32 mL) and H₂O (8 mL) was added LiOH (1.0 M aq. solution, 18.3 mL, 18.3 mmol) dropwise at 0° C. The resulting mixture was stirred for 3 hours at room temperature under an atmosphere of argon. Subsequently, NaHCO₃ (1.69 g, 20.1 mmol) and (Boc)₂O (2.49 g, 11.4 mmol) were added in portions at 0° C. The resulting mixture was stirred for 2 hours at room temperature under an atmosphere of argon. The reaction mixture was quenched by the addition of ice water at 0° C. and was then acidified to pH=4 by the addition of a 1 M aq. citric acid solution, extracted with EtOAc (3×100 mL), dried over anhydrous Na₂SO₄, filtered, and concentrated under reduced pressure to give (2S,3S)-3-(4-bromothiazol-2-yl)-3-((2-(((R)-1-((S)-1-(tert-butoxy)-3-methyl-1-oxobutan-2-yl)pyrrolidin-2-yl)methoxy)benzyl)(methyl)amino)-2-((tert-butoxycarbonyl)amino)propanoic acid (5.00 g, crude) as a yellow oil. This material was taken directly to the next reaction without further purification. LCMS (ESI) m/z: [M+H] calcd for C₃₃H₄₉BrN₄O₇S: 725.3; found 725.4.

Step 10. To a stirred mixture of methyl (S)-hexahydropyridazine-3-carboxylate bis(2,2,2-trifluoroacetate) (8.10 g, 21.8 mmol), DIEA (18.8 g, 145 mmol), and (2S,3S)-3-(4-bromothiazol-2-yl)-3-((2-(((R)-1-((S)-1-(tert-butoxy)-3-methyl-1-oxobutan-2-yl)pyrrolidin-2-yl)methoxy)benzyl)(methyl)amino)-2-((tert-butoxycarbonyl)amino)propanoic acid (5.30 g, crude) in DMF (53 mL) was added HATU (3.06 g, 8.05 mmol) in portions at 0° C. The resulting mixture was stirred for 1 hour at room temperature under an atmosphere of argon. The reaction mixture was then quenched by the addition of sat. aq. NH₄Cl, extracted with EtOAc (100 mL), treated with brine (3×100 mL), dried over anhydrous Na₂SO₄, filtered, concentrated under reduced pressured, and purified by normal phase flash column chromatography to give methyl (S)-1-((2S,3S)-3-(4-bromothiazol-2-yl)-3-((2-(((R)-1-((S)-1-(tert-butoxy)-3-methyl-1-oxobutan-2-yl)pyrrolidin-2-yl)methoxy)benzyl)(methyl)amino)-2-((tert-butoxycarbonyl)amino)propanoyl)hexahydropyridazine-3-carboxylate (3.50 g, 60% yield over 2 steps) as a yellow oil. LCMS (ESI) m/z: [M+H] calcd for C₃₉H₅₉BrN₆O₈S: 851.3; found 851.3.

Step 11. To a stirred solution nof methyl (S)-1-((2S,3S)-3-(4-bromothiazol-2-yl)-3-((2-(((R)-1-((S)-1-(tert-butoxy)-3-methyl-1-oxobutan-2-yl)pyrrolidin-2-yl)methoxy)benzyl)(methyl)amino)-2-((tert-butoxycarbonyl)amino)propanoyl)hexahydropyridazine-3-carboxylate (1.30 g, 1.53 mmol) in DCM (13 mL) was added TFA (13 mL) dropwise at 0° C. The resulting mixture was stirred for 30 hours at room temperature under an atmosphere of argon. The reaction mixture was then concentrated under reduced pressure to give (S)-2-((R)-2-((2-((((1 S,2S)-2-amino-1-(4-bromothiazol-2-yl)-3-((S)-3-(methoxycarbonyl)tetrahydropyridazin-1(2H)-yl)-3-oxopropyl)(methyl)amino)methyl)phenoxy)methyl)pyrrolidin-1-yl)-3-methylbutanoic acid (2.00 g, crude, TFA salt) as a yellow solid. This material was taken directly to the next reaction without further purification. LCMS (ESI) m/z: [M+H] calcd for C₃₀H₄₃BrN₆O₆S: 695.2; found 695.2.

Step 12. To a stirred mixture of (S)-2-((R)-2-((2-((((1S,2S)-2-amino-1-(4-bromothiazol-2-yl)-3-((S)-3-(methoxycarbonyl)tetrahydropyridazin-1(2H)-yl)-3-oxopropyl)(methyl)amino)methyl)phenoxy)methyl)pyrrolidin-1-yl)-3-methylbutanoic acid trifluoroacetic acid salt (2.80 g, crude) and DIEA (18.3 g, 142 mmol) in DMF (280 mL) was added HATU (2.70 g, 7.10 mmol) in portions at 0° C. The resulting mixture was stirred for 2 hours at room temperature under an atmosphere of argon. The reaction mixture was then quenched by the addition of sat. aq. NH₄Cl, extracted with EtOAc (100 mL) treated with brine (3×100 mL), dried over anhydrous Na₂SO₄, filtered, concentrated under reduced pressure, and purified by normal phase prep-TLC to afford methyl (S)-1-((5S,8S,9S,17aR)-9-(4-bromothiazol-2-yl)-5-isopropyl-10-methyl-6-oxo-2,3,6,7,8,9,10,11,17,17a-decahydro-1H,5H-benzo[ ]pyrrolo[2,1-c][1]oxa[4,7,10]triazacyclotridecine-8-carbonyl)hexahydropyridazine-3-carboxylate (800 mg, 55% yield over 2 steps) as a yellow solid. LCMS (ESI) m/z: [M+H] calcd for C₃₀H₄₁BrN₆O₅S: 677.2; found 677.1.

Step 13. To a stirred solution of (S)-3-(2-(5-(4-cyclopropylpiperazin-1-yl)-2-(1-methoxyethyl)pyridin-3-yl)-1-ethyl-5-(4,4,5,5-tetramethyl-1,3,2-dioxaborolan-2-yl)-1H-indol-3-yl)-2,2-dimethylpropan-1-ol (565 mg, 0.916 mmol) and methyl (S)-1-((5S,8S,9S,17aR)-9-(4-bromothiazol-2-yl)-5-isopropyl-10-methyl-6-oxo-2,3,6,7,8,9,10,11,17,17a-decahydro-1H,5H-benzo[l]pyrrolo[2,1-c][1]oxa[4,7,10]triazacyclotridecine-8-carbonyl)hexahydropyridazine-3-carboxylate (218 mg, 0.364 mmol) in 1,4-dioxane (5.6 mL) and H₂O (1.1 mL) were added K₂CO₃ (316 mg, 2.29 mmol) and Pd(dtbpf)Cl₂ (119 mg, 0.183 mmol) in portions at 0° C. The resulting mixture was stirred for 3 hours at 70° C. under an atmosphere of argon. The reaction mixture was quenched with ice water at 0° C., extracted with EtOAc (3×30 mL), dried over anhydrous Na₂SO₄, filtered, concentrated under reduced pressure, and purified by normal phase prep-TLC to afford methyl (S)-1-((5S,8S,9S,17aR)-9-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-5-isopropyl-10-methyl-6-oxo-2,3,6,7,8,9,10,11,17,17a-decahydro-1H,5H-benzo[ ]pyrrolo[2,1-c][1]oxa[4,7,10]triazacyclotridecine-8-carbonyl)hexahydropyridazine-3-carboxylate (560 mg, 51% yield) as a yellow oil. LCMS (ESI) m/z: [M+H] calcd for C₆₀H₈₂N₁₀O₇S: 1087.6 found 1087.5.

Step 14. To a stirred solution of methyl (S)-1-((5S,8S,9S,17aR)-9-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-5-isopropyl-10-methyl-6-oxo-2,3,6,7,8,9,10,11,17,17a-decahydro-1H,5H-benzo[l]pyrrolo[2,1-c][1]oxa[4,7,10]triazacyclotridecine-8-carbonyl)hexahydropyridazine-3-carboxylate (560 mg, 0.515 mmol) in THF (5.6 mL) and H₂O (1.1 mL) was added LiOH·H₂O (43 mg, 1.03 mmol) in portions at 0° C. The resulting mixture was stirred for 1 hour at room temperature under an atmosphere of argon. The reaction mixture was quenched by the addition of ice water 0° C., and was then acidified to pH 5 by the addition of a 1 M aq. citric acid solution, extracted with EtOAc (3×30 mL), dried over anhydrous Na₂SO₄, filtered, and concentrated under reduced pressure to give (S)-1-((5S,8S,9S,17aR)-9-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-5-isopropyl-10-methyl-6-oxo-2,3,6,7,8,9,10,11,17,17a-decahydro-1H,5H-benzo[l]pyrrolo[2,1-c][1]oxa[4,7,10]triazacyclotridecine-8-carbonyl)hexahydropyridazine-3-carboxylic acid (540 mg, crude) as a yellow solid. This material was taken directly to the next step without further purification. LCMS (ESI) m/z: [M+H] calcd for C₅₉H₈₀N₁₀O₇S: 1073.6; found 1073.6.

Step 15. To a stirred mixture of (S)-1-((5S,8S,9S,17aR)-9-(4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-5-isopropyl-10-methyl-6-oxo-2,3,6,7,8,9,10,11,17,17a-decahydro-1H,5H-benzo[ ]pyrrolo[2,1-c][1]oxa[4,7,10]triazacyclotridecine-8-carbonyl)hexahydropyridazine-3-carboxylic acid (540 mg, 0.503 mmol), DIEA (2.60 g, 20.1 mmol) and HOBT (136 mg, 1.01 mmol) in DCM (54 mL) was added EDCI (2.89 g, 15.1 mmol) in portions at 0° C. The resulting mixture was stirred for 16 hours at room temperature under an atmosphere of argon. The reaction mixture was then concentrated under reduced pressure and the resulting material was dissolved in EtOAc (20 mL), treated with sat. aq. NH₄Cl (3×20 mL), dried over anhydrous Na₂SO₄, filtered, and concentrated under reduced pressure. The resulting material was purified by normal phase prep-TLC and was subsequently purified by reversed-phase prep-HPLC to give (3⁵S,3⁸S,3⁹S,3^{17a}R,5³S,Z)-1²-(5-(4-cyclopropylpiperazin-1-yl)-2-((S)-1-methoxyethyl)pyridin-3-yl)-1 ¹-ethyl-3⁵-isopropyl-3¹⁰,9,9-trimethyl-3²,3³,3⁶,3⁷,3⁸,3⁹,3¹⁰,3¹¹,3¹⁷,3^{17a},5¹,5²,5³,5⁴,5⁵,5⁶- hexadecahydro-1¹H,3¹H,3⁵H-7-oxa-3(9,8)-benzo[l]pyrrolo[2,1-c][1]oxa[4,7,10]triazacyclotridecina-2(4,2)-thiazola-1 (5,3)-indola-5(1,3)-pyridazinacyclodecaphane-3⁶,4,6-trione (100 mg, 18% yield over 2 steps) as a white solid. LCMS (ESI) m/z: [M+H] calcd for C₅₉H₇₈N₁₀O₆S: 1055.6; found 1055.5. ¹H NMR (400 MHz, DMSO-d₆) δ 8.82 (d, J=7.0 Hz, 1H), 8.45 (s, 1H), 8.31 (s, 1H) 7.93 (s, 1H), 7.69 (d, J=8.5 Hz, 1H), 7.56 (d, J=8.6 Hz, 1H), 7.30 (t, J=7.9 Hz, 1H), 7.25-7.12 (m, 2H), 7.09 (d, J=8.2 Hz, 1H), 6.91 (t, J=7.4 Hz, 1H), 5.48 (br s, 1H), 5.02 (br s, 1H), 4.31-4.08 (m, 7H), 3.92-3.60 (m, 3H), 3.22 (s, 6H), 3.12-2.91 (m, 4H), 2.81-2.61 (m, 6H), 2.41 (br s, 1H), 2.21 (h, J=6.6 Hz, 2H), 2.09-1.95 (m, 1H), 1.94-1.61 (m, 7H), 1.60-1.40 (m, 2H), 1.33 (d, J=6.1 Hz, 3H), 1.10 (d, J=6.5 Hz, 3H), 1.06-0.66 (m, 9H), 0.44 (d, J=6.2 Hz, 2H), 0.34 (br s, 4H).

TABLE 2
Exemplary Compounds Prepared by
Methods of the Present Invention

| Ex# | MS Found |
|---|---|
| A1 | 980.6 |
| A2 | 980.5 |
| A3 | 986.6 |
| A7 | 1001.6 |
| A8 | 980.8 |
| A9 | 959.8 |
| A10 | 919.6 |
| A11 | 931.6 |
| A12 | 876.5 |
| A13 | 966.6 |
| A14 | 959.7 |
| A15 | 966.6 |
| A16 | 1055.5 |
| A17 | 979.6 |
| A18 | 1007.5 |
| A19 | 1003.5 |
| A20 | 1007.5 |
| A21 | 849.6 |
| A22 | 1023.6 |
| A23 | 995.5 |
| A24 | 987.5 |
| A25 | 987.6 |
| A26 | 990.7 |
| A27 | 1000.6 |
| A28 | 967.7 |
| A29 | 967.7 |
| A30 | 967.6 |
| A31 | 987.6 |
| A32 | 1069.7 |
| A33 | 993.6 |
| A34 | 928.5 |
| A35 | 876.8 |
| A36 | 1009.6 |
| A37 | 941.6 |
| A38 | 1009.7 |
| A39 | 797.4 |
| A40 | 903.6 |
| A41 | 876.7 |
| A42 | 855.7 |
| A43 | 943.6 |
| A44 | 993.7 |
| A45 | 980.7 |
| A46 | 979.6 |
| A47 | 995.7 |
| A48 | 957.5 |
| A49 | 958.5 |
| A50 | 891.7 |
| A51 | 891.7 |
| A52 | 749.6 |
| A53 | 921.8 |
| A54 | 1097.7 |
| A55 | 907.7 |
| A56 | 904.6 |
| A57 | 905.6 |
| A58 | 919.4 |
| A59 | 1111.6 |
| A60 | 1097.6 |
| A61 | 1099.6 |
| A62 | 1071.6 |
| A63 | 1109.7 |
| A64 | 947.6 |
| A65 | 919.5 |
| A66 | 1097.7 |
| A67 | 461.4 |
| A68 | 870.4 |
| A69 | 1099.6 |
| A70 | 1085.5 |
| A71 | 1085.5 |
| A72 | 1085.5 |
| A73 | 1059.7 |
| A75 | 781.4 |
| A76 | 905.5 |
| A77 | 1099.6 |
| A78 | 1085.6 |
| A79 | 1087.6 |
| A80 | 1059.5 |
| A81 | 903.6 |
| A82 | 916.5 |
| A83 | 1097.6 |
| A84 | 1097.6 |
| A85 | 1043.5 |
| A86 | 1043.5 |
| A87 | 907.5 |
| A88 | 792.4 |
| A89 | 903.6 |
| A90 | 862.4 |
| A91 | 780.4 |
| A92 | 903.5 |
| A93 | 1078.6 |
| A94 | 1007.4 |
| A95 | 1055.5 |
| A96 | 956.4 |
| A97 | 902.4 |
| A98 | 808.3 |
| A99 | 773.5 |
| A100 | 877.6 |
| A101 | 1000.6 |
| A102 | 1097.6 |
| A103 | 976.7 |
| A104 | 953.6 |
| A105 | 952.6 |
| A106 | 891.6 |
| A107 | 967.5 |
| A108 | 967.5 |
| A109 | 905.6 |
| A110 | 851.5 |
| A111 | 889.7 |
| A112 | 948.5 |
| A113 | 891.7 |
| A114 | 1016.6 |
| A115 | 1016.6 |
| A116 | 983.7 |
| A117 | 934.7 |
| A118 | 1029.7 |
| A119 | 1029.7 |
| A120 | 891.6 |
| A121 | 977.7 |
| A122 | 935.6 |
| A123 | 931.4 |
| A124 | 1110.6 |
| A125 | 956.5 |
| A126 | 922 |
| A127 | 921.6 |
| A128 | 922.5 |
| A129 | 976.5 |
| A130 | 976.5 |
| A131 | 948.4 |
| A132 | 835.4 |
| A133 | 875.8 |
| A134 | 892.5 |
| A135 | 849.8 |
| A136 | 918.6 |
| A137 | 892.3 |
| A138 | 860.5 |
| A139 | 904.5 |
| A140 | 876.2 |
| A141 | 920.9 |
| A142 | 812.7 |
| A143 | 921.8 |
| A144 | 921.8 |
| A145 | 878.8 |
| A146 | 892.8 |
| A148 | 1002.5 |
| A149 | 878 |
| A150 | 860.5 |
| A151 | 861.4 |
| A152 | 878.7 |
| A153 | 861.3 |
| A154 | 874.3 |
| A155 | 862.5 |
| A156 | 820.4 |
| A157 | 904.5 |
| A158 | 904.3 |
| A159 | 875.4 |
| A160 | 778.4 |
| A161 | 781.3 |
| A162 | 820.4 |
| A163 | 854.4 |
| A164 | 889.3 |
| A165 | 890.2 |
| A166 | 781.3 |
| A167 | 834.4 |
| A168 | 876.4 |
| A169 | 876.4 |
| A170 | 835.3 |
| A171 | 832.3 |
| A172 | 862.4 |
| A173 | 834.4 |
| A174 | 860.3 |
| A175 | 829.3 |
| A176 | 775.4 |
| A177 | 775.4 |
| A178 | 832.5 |
| A179 | 817.3 |
| A180 | 819.4 |
| A181 | 821.4 |
| A182 | 1001.7 |
| A183 | 1015.7 |
| A184 | 1015.6 |
| A185 | 1070.8 |
| A186 | 1032.8 |
| A187 | 961.7 |
| A188 | 1015.6 |
| A189 | 947.7 |
| A190 | 1015.5 |
| A191 | 1016.6 |
| A192 | 1016.6 |
| A193 | 1082.7 |
| A194 | 1028.8 |
| A195 | 1044.5 |
| A196 | 1072.5 |
| A197 | 1029.8 |
| A198 | 1110.8 |
| A199 | 1110.8 |
| A200 | 1056.8 |
| A201 | 1069.6 |
| A202 | 1029.7 |
| A203 | 1027.6 |
| A204 | 1082.5 |
| A205 | 1028.6 |
| A206 | 1072.7 |
| A207 | 1056.6 |
| A208 | 1015.6 |
| A209 | 987.6 |
| A210 | 987.6 |
| A211 | 1027.7 |
| A212 | 961.5 |
| A213 | 961.6 |
| A214 | 1084.7 |
| A215 | 1084.7 |
| A216 | 987.7 |
| A217 | 934.7 |
| A218 | 934.6 |
| A219 | 948.5 |
| A220 | 1030.6 |
| A221 | 1030.6 |
| A222 | 1096.6 |
| A223 | 1042.7 |
| A224 | 1042.7 |
| A225 | 1058.6 |
| A226 | 987.4 |
| A227 | 1096.8 |
| A228 | 1042.6 |
| A229 | 1042.7 |
| A230 | 1058.6 |
| A231 | 987.5 |
| A232 | 1124.8 |
| A233 | 1124.7 |
| A234 | 1124.7 |
| A235 | 1124.6 |
| A236 | 1086.5 |
| A237 | 975.5 |
| A238 | 1015.6 |
| A239 | 1015.6 |
| A240 | 1096.6 |
| A241 | 1096.7 |
| A242 | 1070.6 |
| A243 | 1070.5 |
| A244 | 1086.8 |
| A245 | 1015.6 |
| A246 | 1070.7 |
| A247 | 1070.6 |
| A248 | 1015.5 |
| A249 | 1044.5 |
| A250 | 1058.6 |
| A251 | 1058.6 |
| A252 | 1058.5 |
| A253 | 1072.6 |
| A254 | 1072.6 |
| A255 | 1072.5 |
| A256 | 975.6 |
| A257 | 987.6 |
| A258 | 1029.5 |
| A259 | 1043.6 |
| A260 | 1043.7 |
| A261 | 1044.5 |
| A262 | 1044.6 |
| A263 | 1058.5 |
| A264 | 1072.6 |
| A265 | 1003.5 |
| A266 | 1073.8 |
| A267 | 1074.8 |
| A268 | 1074.7 |
| A269 | 1099.8 |
| A270 | 1113.8 |
| A271 | 1000.6 |
| A272 | 1052.6 |
| A273 | 1000.7 |
| A274 | 1013.6 |
| A275 | 1052.7 |
| A276 | 1055.6 |
| A277 | 1055.6 |
| A278 | 877.6 |
| A279 | 1052.8 |
| A280 | 1052.6 |
| A281 | 1081.6 |
| A282 | 1081.6 |
| A283 | 1029.5 |
| A284 | 1001.6 |
| A285 | 1002.8 |
| A286 | 1018.6 |
| A287 | 960.6 |
| A288 | 947.6 |
| A289 | 976 |
| A290 | 975.3 |
| A291 | 1002.6 |

### Instrumentation

Mass spectrometry data collection took place with a Shimadzu LCMS-2020, an Agilent 12601LC-6120/6125MSD, a Shimadzu LCMS-2010EV, or a Waters Acquity UPLC, with either a QDa detector or SQ Detector 2. Samples were injected in their liquid phase onto a 0-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Biological Assays

Disruption of B-Raf Ras-Binding Domain (BRAF^{RBD}) Interaction with K-Ras by Compounds of the Invention (Also Called a FRET Assay or an MOA Assay)

The purpose of this biochemical assay is to measure the ability of test compounds to facilitate ternary complex formation between a nucleotide-loaded K-Ras isoform and cyclophilin A; the resulting ternary complex disrupts binding to a BRAF^{RBD} Construct, inhibiting K-Ras signaling through a RAF effector. Data is reported as IC50 values. Other Ras variants may be used.

In assay buffer containing 25 mM HEPES pH 7.3, 0.002% Tween20, 0.1% BSA, 100 mM NaCl and 5 mM MgCl₂, tagless cyclophilin A, His6-K-Ras-GMPPNP, and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a K-Ras:RAF complex are identified as those eliciting a decrease in the TR-FRET ratio relative to DMSO control wells.

Each of Examples A1-A181 exhibited an IC50 of less than 2 μM with respect to at least one of the following: K-Ras Q61H, G12C, G12D, G12R, G12S, G12V, G12A, G13C, G13D and wild-type; N-Ras Q61K, Q61R, Q61L, G12C and wild-type; and H-Ras G13R and WT.

### Enumerated Embodiments

E1. A compound, or a pharmaceutically acceptable salt thereof, having the structure of Formula Ia-1:

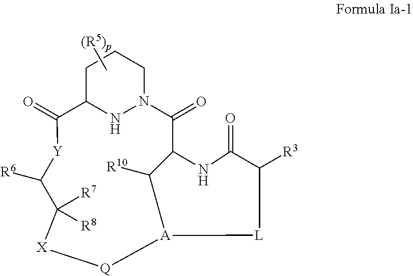

wherein:
Q is an optionally substituted 7- to 12-membered bicyclic arylene, an optionally substituted 7- to 12-membered bicyclic heteroarylene, an optionally substituted 7- to 12-membered bicyclic heterocyclylene, wherein a first ring in Q is bonded to X, and a second ring in Q is bonded to A;
X is a bond; a straight chain C₁-C₃ alkylene optionally substituted with 1 to 3 substituents independently selected from fluoro, —CN, —C₁-C₃ alkyl, and —O—C₁-C₃ alkyl; —O—; —S(O)₀₋₂—; *—CH₂—O—; *—CH₂—S(O)₀₋₂—; *—O—CH₂—; or *—CH₂—S(O)₀₋₂—, wherein “*” represents a portion of X bound to —C(R⁷)(R⁸)—;
Y is —O—, —NH— or —N(C₁-C₃ alkyl)-;
A is optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
L is a linker;
R³ is optionally substituted C₁-C₆ alkyl, optionally substituted C₃-C₆ cycloalkyl, optionally substituted C₆ aryl, or optionally substituted 3- to 7-membered heterocyclyl;
R¹⁰ is hydrogen, halogen, optionally substituted C₁-C₃ alkyl, or C₁-C₃ optionally substituted heteroalkyl;
R⁷ is hydrogen, halogen, or optionally substituted C₁-C₃ alkyl;
R⁸ is hydrogen, halogen, —OH, —CN, —O-(optionally substituted C₁-C₃ alkyl), optionally substituted C₁-C₃ alkyl, optionally substituted C₂-C₆ alkenyl, optionally substituted C₂-C₆ alkynyl, optionally substituted C₆-C₁₀ aryl, optionally substituted 4- to 8-membered heteroaryl, optionally substituted C₃-C₆ cycloalkyl, or optionally substituted 3- to 7-membered heterocyclyl; or
R⁷ and R⁸ are taken together to form ═CH₂, an optionally substituted C₃-C₆ cycloalkyl, or a 3- to 7-membered saturated heterocyclyl; or
R⁸ is taken together with a ring atom in Q, the carbon atom to which R⁷ is bound and X to form a 4- to 9-membered saturated or unsaturated heterocyclyl that is fused to Q;
R⁶ is hydrogen or —CH₃;
each R⁵ is independently halogen, optionally substituted C₁-C₃ alkyl, or optionally substituted
C₁-C₃ haloalkyl; and
p is 0, 1, 2, or 3.

E2. A compound, or a pharmaceutically acceptable salt thereof, having the structure of Formula IIa-2:

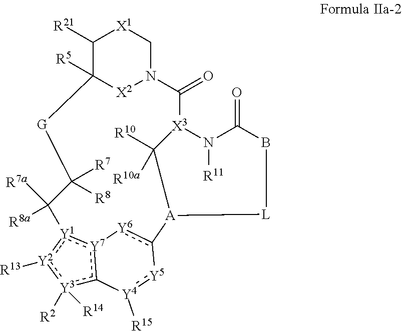

wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl; R¹⁴ is absent or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl; and
R²¹ is hydrogen or optionally substituted C₁-C₃ alkyl

E3. A compound, or a pharmaceutically acceptable salt thereof, having the structure of Formula IIIa-2:

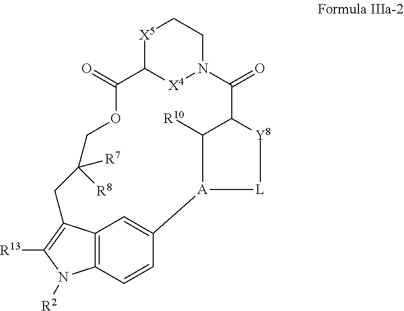

wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is

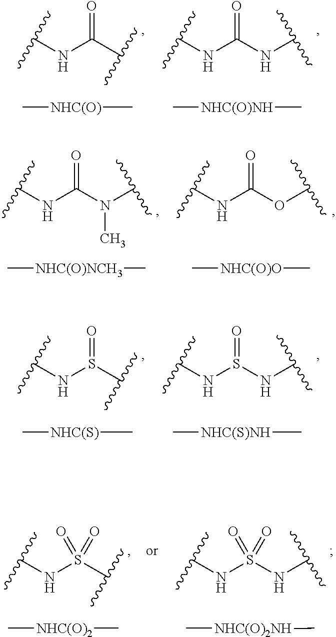

L is a linker;
X⁴ and X⁵ are each, independently, CH₂, CH(CH₃) or NH;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl; and
R⁷ and R⁸ are each, independently, selected from F or CH₃, or R⁷ and R⁸ combine with the atoms to which they are attached to make a 3-membered cycloalkyl.

E4. A compound, or pharmaceutically acceptable salt thereof, having the structure of Formula IVa-3:

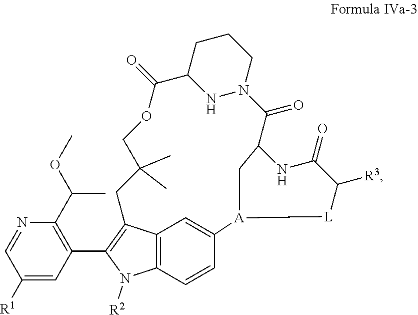

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted 3- to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

E5. A compound, or pharmaceutically acceptable salt thereof, having the structure of Formula IVa-1 or Formula IVb-1:

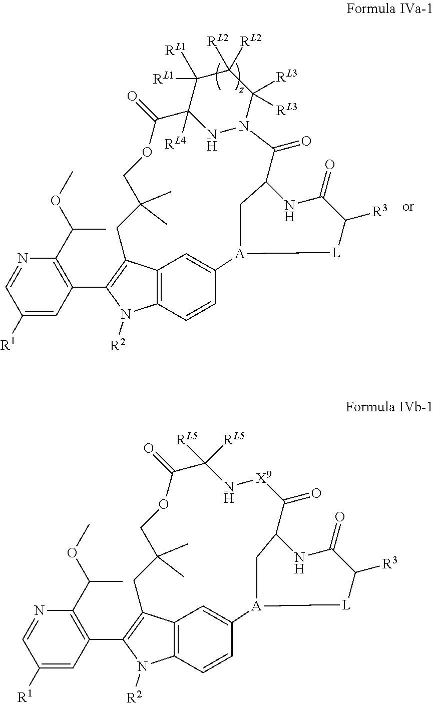

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

E6. A compound, or pharmaceutically acceptable salt thereof, having the structure of Formula IVa-2:

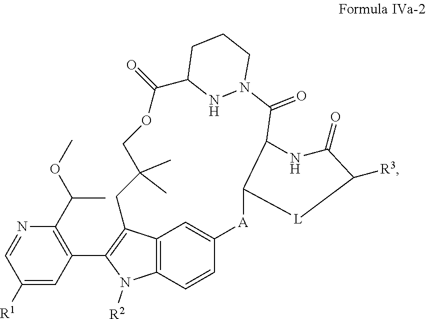

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted 3- to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

E7. A compound, or a pharmaceutically acceptable salt thereof, having the structure of Formula VIa-2:

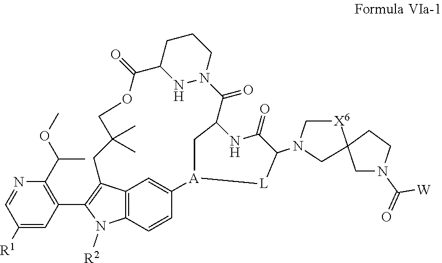

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X⁶ is CH₂ or O;
m is 1 or 2;
n is 0 or 1;
R¹ is hydrogen or optionally substituted 3- to 10-membered heterocycloalkyl; and
R² is optionally substituted C₁-C₆ alkyl.

E8. A compound, or a pharmaceutically acceptable salt thereof, having the structure of Formula VIIa-2:

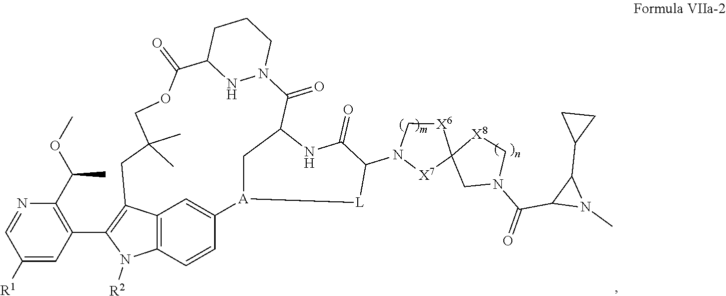

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
X⁶, X⁷, and X⁸ are each independently selected from CH₂, CHF, CF₂, C═O, or O;
m is 1 or 2;
n is 0 or 1;
R¹ is hydrogen, optionally substituted C₁-C₆ heteroalkyl, or optionally substituted 3- to 10-membered heterocycloalkyl;
R² is optionally substituted C₁-C₆ alkyl; and
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium.

E9. The compound of any one of embodiments 1 to 8, or a pharmaceutically acceptable salt thereof, wherein the linker has the structure of Formula XIII:

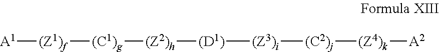

wherein A¹ is a bond between the linker and the rest of the macrocycle; A² is a bond between A and the linker; Z¹, Z², Z³, and Z⁴ are each, independently, optionally substituted C₁-C₃ alkylene, optionally substituted C₁-C₃ heteroalkylene, optionally substituted C₁-C₂ alkenylene, O, NR^{N} or a cross-linking group comprising a vinyl ketone, an ynone, a vinyl sulfone, an alkynyl sulfone, a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal; R^{N} is hydrogen or optionally substituted C₁-C₄ alkyl; C₁ and C² are each, independently, carbonyl or O; f, g, h, i, j, and k are each, independently, 0 or 1; and D¹ is optionally substituted C₁-C₂ alkylene, optionally substituted C₂-C₆ alkenylene, optionally substituted C₂-C₆ alkynylene, optionally substituted 3- to 8-membered heterocycloalkylene, optionally substituted 3- to 8-membered cycloalkylene, or optionally substituted C₁-C₃ heteroalkylene, or a chemical bond linking A¹-(Z¹)_{f}—(C¹)_{g}—(Z²)ₕ— to —(Z³)ᵢ(C²)ⱼ—(Z⁴)ₖ-A².

E10. The compound of any one of embodiments 1 to 8, or a pharmaceutically acceptable salt thereof, wherein the linker has the structure of Formula XIII:

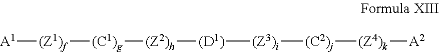

wherein A¹ is a bond between the linker and the rest of the macrocycle; A² is a bond between A and the linker; Z¹, Z², Z³, and Z⁴ are each, independently, optionally substituted C₁-C₃ alkylene, optionally substituted C₁-C₃ heteroalkylene, optionally substituted C₁-C₂ alkenylene, optionally substituted 3- to 8-membered heterocycloalkylene, optionally substituted 3- to 8-membered cycloalkylene, O, NR^{N} or a cross-linking group comprising a vinyl ketone, an ynone, a vinyl sulfone, an alkynyl sulfone, a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal; R^{N} is hydrogen, optionally substituted C₁-C₄ alkyl, or optionally substituted 6-membered arylene; C¹ and C² are each, independently, carbonyl or O; f, g, h, i, j, and k are each, independently, 0 or 1; and D¹ is optionally substituted C₁-C₂ alkylene, optionally substituted C₂-C₆ alkenylene, optionally substituted C₂-C₆ alkynylene, optionally substituted 3- to 8-membered heterocycloalkylene, optionally substituted 3- to 8-membered cycloalkylene, or optionally substituted C₁-C₃ heteroalkylene, optionally substituted 6-membered arylene, or optionally substituted 5- to 10-membered heteroarylene, or a chemical bond linking A¹-(Z¹)_{f}—(C¹)_{g}—(Z²)ₕ— to —(Z³)ᵢ—(C²)ⱼ—(Z⁴)ₖ-A².

E11. The compound of embodiment 9 or 10, or a pharmaceutically acceptable salt thereof, wherein f is 0.

E12. The compound of embodiment 9 or 10, or a pharmaceutically acceptable salt thereof, wherein f is 1.

E13. The compound of any one of embodiments 9-12, or a pharmaceutically acceptable salt thereof, wherein g is 0.

E14. The compound of any one of embodiments 9-12, or a pharmaceutically acceptable salt thereof, wherein g is 1.

E15. The compound of any one of embodiments 9-14, or a pharmaceutically acceptable salt thereof, wherein h is 0.

E16. The compound of any one of embodiments 9-14, or a pharmaceutically acceptable salt thereof, wherein h is 1.

E17. The compound of any one of embodiments 9-16, or a pharmaceutically acceptable salt thereof, wherein i is 0.

E18. The compound of any one of embodiments 9-16, or a pharmaceutically acceptable salt thereof, wherein j is 1.

E19. The compound of any one of embodiments 9-18, or a pharmaceutically acceptable salt thereof, wherein k is 0.

E20. The compound of any one of embodiments 9-18, or a pharmaceutically acceptable salt thereof, wherein k is 1.

E21. The compound of any one of embodiments 9-20, or a pharmaceutically acceptable salt thereof, wherein Z¹ is NR^{N}.

E22. The compound of any one of embodiments 9-20, or a pharmaceutically acceptable salt thereof, wherein R^{N} is optionally substituted C₁-C₄ alkyl.

E23. The compound of any one of embodiments 9-22, or a pharmaceutically acceptable salt thereof, wherein R^{N} is methyl.

E24. The compound of any one of embodiments 9-23, or a pharmaceutically acceptable salt thereof, wherein C¹ is carbonyl.

E25. The compound of any one of embodiments 9-24, or a pharmaceutically acceptable salt thereof, wherein D¹ is 3- to 8-membered cycloalkylene.

E26. The compound of any one of embodiments 9-24, or a pharmaceutically acceptable salt thereof, wherein D¹ is optionally substituted C₁-C₂ alkylene, optionally substituted C₂-C₆ alkenylene, optionally substituted C₂-C₆ alkynylene, or optionally substituted C₁-C₃ heteroalkylene.

E27. The compound of any one of embodiments 9-24, or a pharmaceutically acceptable salt thereof, wherein D¹ is optionally substituted 3- to 8-membered heterocycloalkylene.

E28. The compound of any one of embodiments 9-27, or a pharmaceutically acceptable salt thereof, wherein Z⁴ is O.

E29. The compound of any one of embodiments 9-27, or a pharmaceutically acceptable salt thereof, wherein Z⁴ is optionally substituted C₁-C₃ alkylene.

E30. The compound of any one of embodiments 9-29, or a pharmaceutically acceptable salt thereof, wherein Z³ is optionally substituted C₁-C₃ alkylene.

E31. The compound of any one of embodiments 1-8, or a pharmaceutically acceptable salt thereof, wherein the linker has the structure of Formula VIII:

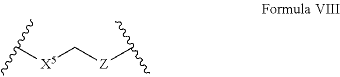

wherein X⁵ is O or CH₂ and is attached to ring A; and
Z is optionally substituted 3- to 6-membered heterocycloalkylene, optionally substituted C₁-C₆ alkylene, or optionally substituted C₁-C₆ heteroalkylene.

E32. The compound of embodiment 31, or a pharmaceutically acceptable salt thereof, wherein X⁵ is O.

E33. The compound of embodiment 31 or 32, or a pharmaceutically acceptable salt thereof, wherein Z is optionally substituted 3- to 6-membered heterocycloalkylene.

E34. The compound of embodiment 31 or 32, or a pharmaceutically acceptable salt thereof, wherein Z is optionally substituted 3- to 6-membered heterocycloalkylene.

E35. The compound of embodiment 31, or a pharmaceutically acceptable salt thereof, wherein Z is optionally substituted 5-membered heterocycloalkylene.

E36. The compound of embodiment 35, or a pharmaceutically acceptable salt thereof, wherein Z is optionally substituted pyrollidine-diyl.

E37. The compound of any one of embodiments 1-8, or a pharmaceutically acceptable salt thereof, wherein the linker has the structure of Formula VIIIa:

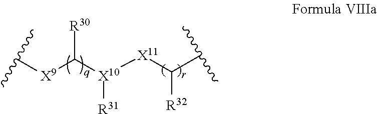

wherein X⁹ is NR, O, or CH₂ and is attached to ring A;
X¹⁰ is CH or N;
X¹¹ is NR″, O, C(O), C(O)N(R″′)₂, or CH₂;
R′ is hydrogen, cyano, optionally substituted C₁-C₄ alkyl, optionally substituted C₂-C₄ alkenyl, optionally substituted C₂-C₄ alkynyl, C(O)R″′, C(O)OR″′, C(O)N(R″′)₂, S(O)R″′, S(O)₂R″′, or S(O)₂N(R″′)₂;
each R″′ is, independently, hydrogen, optionally substituted C₁-C₄ alkyl, or optionally substituted 3- to 6-membered heterocycloalkylene;
R³⁰ and R³² are, independently, hydrogen, optionally substituted C₆-C₁₀ aryl, or optionally substituted C₁-C₆ alkylene;
R³¹ is hydrogen, optionally substituted C₆-C₁₀ aryl, optionally substituted 4- to 8-membered heteroaryl, optionally substituted 3- to 6-membered cycloalkylene, optionally substituted 3- to 6-membered heterocycloalkylene, optionally substituted C₁-C₆ alkylene, or optionally substituted C₁-C₆ heteroalkylene; and
q and r are, independently, 0, 1, 2, or 3.

E38. The compound of embodiment 37, or a pharmaceutically acceptable salt thereof, wherein the linker is:

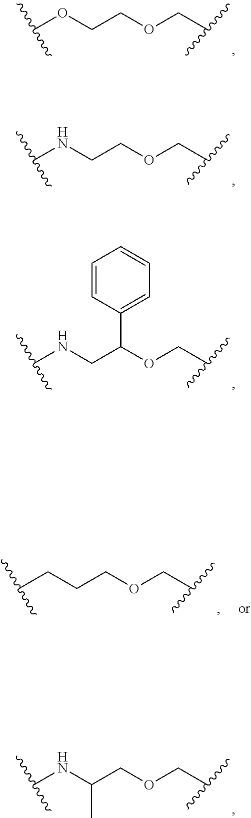

E39. The compound of any one of embodiments 1-8, or a pharmaceutically acceptable salt thereof, wherein the linker has the structure of Formula IX:

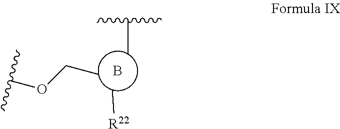

wherein
B is an optionally substituted 3- to 6-membered heterocycloalkylene;
R²² is hydrogen, optionally substituted C₁-C₆ alkyl, optionally substituted C₁-C₆ heteroalkyl, optionally substituted optionally substituted 3- to 6-membered heterocyclyl, optionally substituted 3- to 6-membered cycloalkyl, optionally substituted 5- to 10-membered heteroaryl, optionally substituted C₆-C₁₀ aryl,

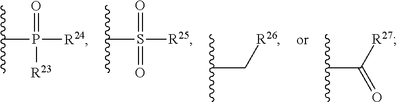

R²³ and R²⁴ are each, independently, hydrogen or optionally substituted C₁-C₆ alkyl;
R²⁵ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R²⁶ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R²⁷ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

E40. The compound of embodiment 39, or a pharmaceutically acceptable salt thereof, wherein the linker has the structure of Formula X:

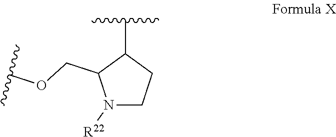

E41. The compound of embodiment 39 or 40, or a pharmaceutically acceptable salt thereof, wherein R²² is

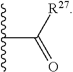

E42. The compound of any one of embodiments 39 to 41, or a pharmaceutically acceptable salt thereof, wherein R²⁷ is optionally substituted C₁-C₆ alkyl.

E43. The compound of any one of embodiments 39 to 41, or a pharmaceutically acceptable salt thereof, wherein R²⁷ is optionally substituted C₂-C₆ alkenyl.

E44. The compound of any one of embodiments 39 to 41, or a pharmaceutically acceptable salt thereof, wherein R²⁷ is optionally substituted C₂-C₆ alkynyl.

E45. The compound of any one of embodiments 39 to 41, or a pharmaceutically acceptable salt thereof, wherein R²⁷ is optionally substituted C₁-C₆ heteroalkyl.

E46. The compound of any one of embodiments 39 to 41, or a pharmaceutically acceptable salt thereof, wherein R²⁷ is optionally substituted C₂-C₆ heteroalkenyl.

E47. The compound of any one of embodiments 39 to 41, or a pharmaceutically acceptable salt thereof, wherein R²⁷ is optionally substituted C₂-C₆ heteroalkynyl.

E48. The compound of any one of embodiments 39 to 41, or a pharmaceutically acceptable salt thereof, wherein R²⁷ is optionally substituted C₃-C₁₀ cycloalkenyl.

E49. The compound of any one of embodiments 39 to 41, or a pharmaceutically acceptable salt thereof, wherein R²⁷ is hydrogen.

E50. The compound of any one of embodiments 39 to 41, or a pharmaceutically acceptable salt thereof, wherein R²⁷ is optionally substituted C₃-C₁₀ cycloalkyl.

E51. The compound of any one of embodiments 39 to 41, or a pharmaceutically acceptable salt thereof, wherein R²⁷ is optionally substituted 3- to 10-membered heterocyclyl.

E52. The compound of embodiment 39 or 40, or a pharmaceutically acceptable salt thereof, wherein R²² is

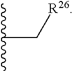

E53. The compound of any one of embodiments 39, 40, and 52, or a pharmaceutically acceptable salt thereof, wherein R²⁶ is optionally substituted 5- to 10-membered heteroaryl.

E54. The compound of any one of embodiments 39, 40, and 52, or a pharmaceutically acceptable salt thereof, wherein R²⁶ is optionally substituted 3- to 10-membered heterocyclyl.

E55. The compound of embodiment 39 or 40, or a pharmaceutically acceptable salt thereof, wherein R²² is optionally substituted optionally substituted 3- to 6-membered heterocyclyl.

E56. The compound of embodiment 39 or 40, or a pharmaceutically acceptable salt thereof, wherein R²² is optionally substituted 3- to 6-membered heterocyclyl.

E57. The compound of any one of embodiments 1 to 56, or a pharmaceutically acceptable salt thereof, wherein A is optionally substituted 3- to 6-membered heterocycloalkylene, optionally substituted 6-membered arylene, or optionally substituted 5- to 10-membered heteroarylene.

E58. The compound of any one of embodiments 1 to 56, or a pharmaceutically acceptable salt thereof, wherein A is optionally substituted 6-membered arylene.

E59. The compound of any one of embodiments 1 to 58, or a pharmaceutically acceptable salt thereof, wherein A is optionally substituted 3- to 6-membered heterocycloalkylene, optionally substituted 6-membered arylene, or optionally substituted 5- to 10-membered heteroarylene.

E60. The compound of any one of embodiments 1 to 59, or a pharmaceutically acceptable salt thereof, wherein A is:

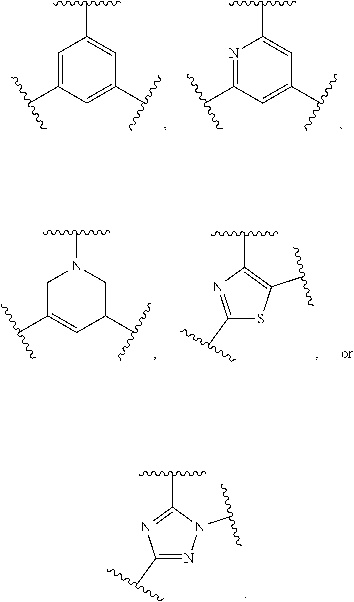 [SMILES: *c1cc(*)cc(*)c1 ; *c1cc(*)nc(*)c1 ; *C1=CC(*)CN(*)C1 ; *c1nc(*)c(*)s1 ; *c1nc(*)n(*)n1]

E61. The compound of embodiment 58, or a pharmaceutically acceptable salt thereof, wherein A is

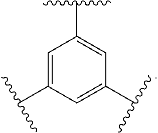 [SMILES: *c1cc(*)cc(*)c1]

E62. A compound, or a pharmaceutically acceptable salt thereof, of Table 1.

E63. A pharmaceutical composition comprising a compound, or a pharmaceutically acceptable salt thereof, of any one of embodiments 1-62 and a pharmaceutically acceptable excipient.

E64. A method of treating cancer in a subject in need thereof, the method comprising administering to the subject a therapeutically effective amount of a compound, or a pharmaceutically acceptable salt thereof, of any one of embodiments 1-62 or a pharmaceutical composition of embodiment 63.

E65. The method of embodiment 64, wherein the cancer is pancreatic cancer, colorectal cancer, non-small cell lung cancer, or endometrial cancer.

E66. The method of embodiment 64 or 65, wherein the cancer comprises a Ras mutation.

E67. A method of treating a Ras protein-related disorder in a subject in need thereof, the method comprising administering to the subject a therapeutically effective amount of a compound of any one of embodiments 1-62, or a pharmaceutically acceptable salt thereof, or a pharmaceutical composition of embodiment 63.

E68. A method of inhibiting a Ras protein in a cell, the method comprising contacting the cell with an effective amount of a compound of any one of embodiments 1 to 62, or a pharmaceutically acceptable salt thereof, or a pharmaceutical composition of embodiment 63.

E69. The method of embodiment 67 or 68, wherein the Ras protein K-Ras.

E70. The method of embodiment 68 or 69, wherein the cell is a cancer cell.

E71. The method of embodiment 70, wherein the cancer cell is a pancreatic cancer cell, a colorectal cancer cell, a non-small cell lung cancer cell, or an endometrial cancer cell.

E72. The method of any one of embodiments 68-71, wherein the method further comprises administering an additional anti-cancer therapy.

E73. The method of embodiment 72, wherein the additional anti-cancer therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof.

E74. The method of embodiment 72 or 73, wherein the additional anti-cancer therapy is a SHP2 inhibitor.

1. A compound selected from Formula Ia, Formula Ib, Formula IIa, Formula IIb, Formula IIa-1, Formula IIIa, Formula IIIb, Formula IIIa-1, Formula IVa, Formula IVb, Formula Va, Formula Vb, Formula VIa, Formula VIb, Formula VIIa, Formula VIIb, or Formula XI, or a pharmaceutically acceptable salt thereof,
wherein Formula Ia has the structure of: 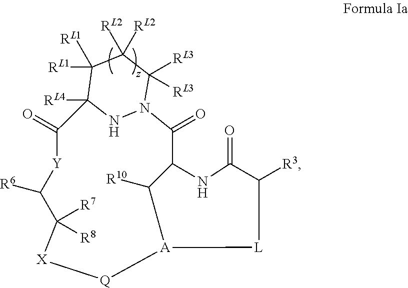
wherein:
Q is an optionally substituted 7- to 12-membered bicyclic arylene, an optionally substituted 7- to 12-membered bicyclic heteroarylene, an optionally substituted 7- to 12-membered bicyclic heterocyclylene, wherein a first ring in Q is bonded to X, and a second ring in Q is bonded to A;
X is a bond; a straight chain C₁-C₃ alkylene optionally substituted with 1 to 3 substituents independently selected from fluoro, —CN, —C₁-C₃ alkyl, and —O—C₁-C₃ alkyl; —O—; —S(O)₀₋₂—; *—CH₂—O—; *—CH₂—S(O)₀₋₂—; *—O—CH₂—; or *—CH₂—S(O)₀₋₂—, wherein “*” represents a portion of X bound to —C(R⁷)(R⁸)—;
Y is —O—, —NH— or —N(C₁-C₃ alkyl)-;
A is optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
L is a linker;
R³ is optionally substituted C₁-C₆ alkyl, optionally substituted C₃-C₆ cycloalkyl, optionally substituted C₆ aryl, or optionally substituted 3- to 7-membered heterocyclyl;
R¹⁰ is hydrogen, halogen, optionally substituted C₁-C₃ alkyl, or C₁-C₃ optionally substituted heteroalkyl;
R⁷ is hydrogen, halogen, or optionally substituted C₁-C₃ alkyl;
R⁸ is hydrogen, halogen, —OH, —CN, —O-(optionally substituted C₁-C₃ alkyl), optionally substituted C₁-C₃ alkyl, optionally substituted C₂-C₆ alkenyl, optionally substituted C₂-C₆ alkynyl, optionally substituted C₆-C₁₀ aryl, optionally substituted 4- to 8-membered heteroaryl, optionally substituted C₃-C₆ cycloalkyl, or optionally substituted 3- to 7-membered heterocyclyl; or
R⁷ and R⁸ together form ═CH₂, an optionally substituted C₃-C₆ cycloalkyl, or a 3- to 7-membered saturated heterocyclyl; or
R⁸ and a ring atom in Q, the carbon atom to which R⁷ is bound, and X to form a 4- to 9-membered saturated or unsaturated heterocyclyl that is fused to Q;
R⁶ is hydrogen or —CH₃;
each R⁵ is independently halogen, optionally substituted C₁-C₃ alkyl, or optionally substituted
C₁-C₃ haloalkyl;
p is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
wherein Formula Ib has the structure of: 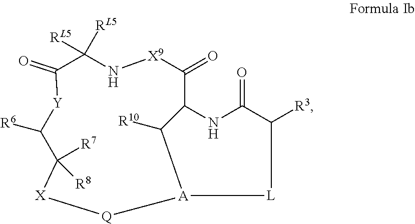
wherein:
Q is an optionally substituted 7- to 12-membered bicyclic arylene, an optionally substituted 7- to 12-membered bicyclic heteroarylene, an optionally substituted 7- to 12-membered bicyclic heterocyclylene, wherein a first ring in Q is bonded to X, and a second ring in Q is bonded to A;
X is a bond; a straight chain C₁-C₃ alkylene optionally substituted with 1 to 3 substituents independently selected from fluoro, —CN, —C₁-C₃ alkyl, and —O—C₁-C₃ alkyl; —O—; —S(O)₀₋₂—; *—CH₂—O—; *—CH₂—S(O)₀₋₂—; *—O—CH₂—; or *—CH₂—S(O)₀₋₂—, wherein “*” represents a portion of X bound to —C(R⁷)(R⁸)—;
Y is —O—, —NH— or —N(C₁-C₃ alkyl)-;
A is optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
L is a linker;
R³ is optionally substituted C₁-C₆ alkyl, optionally substituted C₃-C₆ cycloalkyl, optionally substituted C₆ aryl, or optionally substituted 3- to 7-membered heterocyclyl;
R¹⁰ is hydrogen, halogen, optionally substituted C₁-C₃ alkyl, or C₁-C₃ optionally substituted heteroalkyl;
R⁷ is hydrogen, halogen, or optionally substituted C₁-C₃ alkyl;
R⁸ is hydrogen, halogen, —OH, —CN, —O-(optionally substituted C₁-C₃ alkyl), optionally substituted C₁-C₃ alkyl, optionally substituted C₂-C₆ alkenyl, optionally substituted C₂-C₆ alkynyl, optionally substituted C₆-C₁₀ aryl, optionally substituted 4- to 8-membered heteroaryl, optionally substituted C₃-C₆ cycloalkyl, or optionally substituted 3- to 7-membered heterocyclyl; or
R⁷ and R⁸ together form ═CH₂, an optionally substituted C₃-C₆ cycloalkyl, or a 3- to 7-membered saturated heterocyclyl; or
R⁸ and a ring atom in Q, the carbon atom to which R⁷ is bound, and X to form a 4- to 9-membered saturated or unsaturated heterocyclyl that is fused to Q;
R⁶ is hydrogen or —CH₃;
each R⁵ is independently halogen, optionally substituted C₁-C₃ alkyl, or optionally substituted
C₁-C₃ haloalkyl;
p is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
wherein Formula IIa has the structure of: 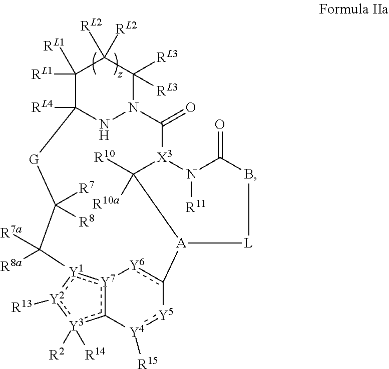
or a pharmaceutically acceptable salt thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CR^{x}, CH, CH₂, or N;
Y⁶ is CR^{z}, C(O), CH, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{z} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl;
R²¹ is hydrogen or optionally substituted C₁-C₃ alkyl;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
wherein Formula IIb has the structure of: 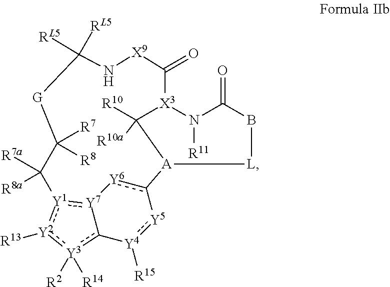
or a pharmaceutically acceptable salt thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CR^{x}, CH, CH₂, or N;
Y⁶ is CR^{z}, C(O), CH, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{z} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl;
R²¹ is hydrogen or optionally substituted C₁-C₃ alkyl;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
wherein Formula IIa-1 has the structure of: 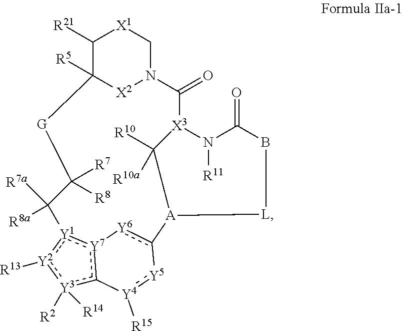
or a pharmaceutically acceptable salt thereof, wherein the dotted lines represent zero, one, two, three, or four non-adjacent double bonds;
A is optionally substituted C₂-C₄ alkylene, optionally substituted C₁-C₄ heteroalkylene, optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3- to 6-membered cycloalkylene, optionally substituted 3- to 6-membered heterocycloalkylene, optionally substituted 6-membered arylene, or optionally substituted 5- to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3- to 8-membered heteroarylene;
L is a linker;
X¹ is optionally substituted C₁-C₂ alkylene, NR, O, or S(O)_{q};
X² is O or NH;
X³ is N or CH;
q is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, hydrogen or optionally substituted C₁-C₄ alkyl;
Y¹ is C, CH, or N;
Y², Y³, Y⁴, and Y⁷ are, independently, C or N;
Y⁵ is CR^{x}, CH, CH₂, or N;
Y⁶ is CR^{z}, C(O), CH, CH₂, or N;
R^{x} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R^{z} is hydrogen, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
R¹³ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6- to 10-membered aryl, or optionally substituted 5- to 10-membered heteroaryl, or
R¹³ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁴ is absent, or R² and R¹⁴ combine with the atom to which they are attached to form an optionally substituted 3- to 8-membered cycloalkyl or optionally substituted 3- to 14-membered heterocycloalkyl;
R¹⁵ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ heteroalkyl, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5 to 10-membered heteroaryl, or optionally substituted 6- to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halogen, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ heteroalkyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3 to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl; or
R⁹ and L combine with the atoms to which they are attached to form an optionally substituted 3- to 14-membered heterocycloalkyl;
R^{9′} is hydrogen or optionally substituted C₁-C₆ alkyl; or
R⁹ and R^{9′}, combined with the atoms to which they are attached, form a 3- to 6-membered cycloalkyl or a 3- to 6-membered heterocycloalkyl;
R¹⁰ is hydrogen, halogen, hydroxy, optionally substituted C₁-C₃ heteroalkyl, or optionally substituted C₁-C₃ alkyl;
R^{10a} is hydrogen or halogen;
R¹¹ is hydrogen or optionally substituted C₁-C₃ alkyl; and
R²¹ is hydrogen or optionally substituted C₁-C₃ alkyl;
wherein Formula IIIa has the structure of: 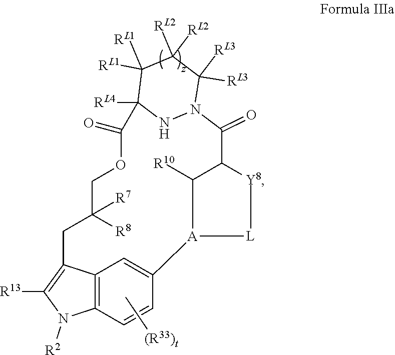
or a pharmaceutically acceptable salt thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is 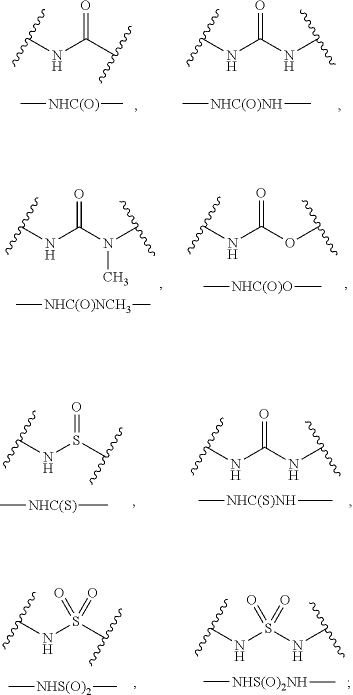
L is a linker;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl;
R⁷ and R⁸ are each, independently, selected from F or CH₃, or R⁷ and R⁸ combine with the atoms to which they are attached to make a 3-membered cycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
t is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
wherein Formula IIIb has the structure of: 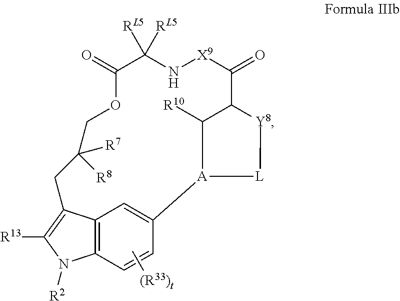
or a pharmaceutically acceptable salt thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is 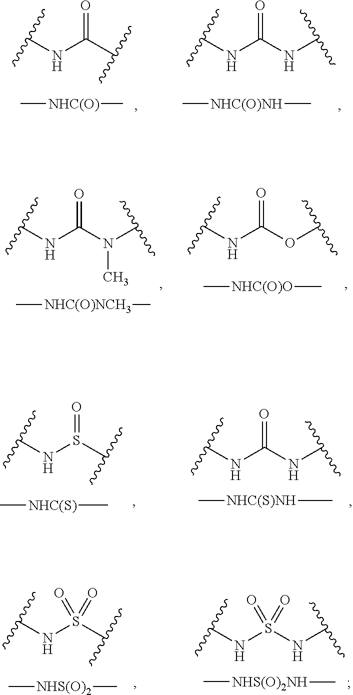
L is a linker;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl;
R⁷ and R⁸ are each, independently, selected from F or CH₃, or R⁷ and R⁸ combine with the atoms to which they are attached to make a 3-membered cycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl;
t is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
wherein Formula IIIa-1 has the structure of: 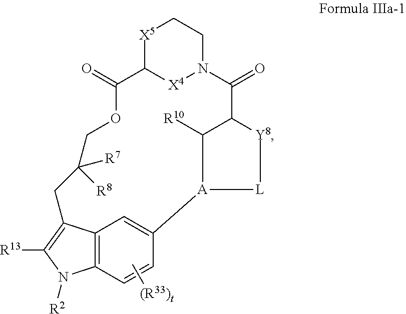
or a pharmaceutically acceptable salt thereof, wherein A is optionally substituted 3- to 6-membered cycloalkylene, optionally substituted 3- to 6-membered heterocycloalkylene, optionally substituted 6-membered arylene, optionally substituted 5- to 6-membered heteroarylene, optionally substituted C₂-C₄ alkylene, optionally substituted C₁-C₄ heteroalkylene or optionally substituted C₂-C₄ alkenylene;
Y⁸ is 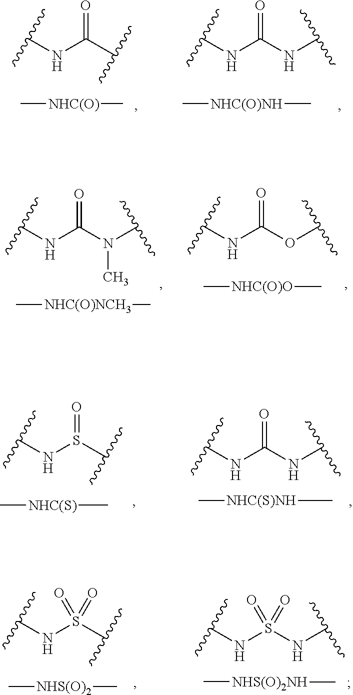
L is a linker;
X⁴ and X⁵ are each, independently, CH₂, CH(CH₃) or NH;
R¹³ is optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 15-membered heterocycloalkyl, optionally substituted 6- to 10-membered aryl, or optionally substituted 5- to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3- to 7-membered heterocycloalkyl;
R⁷ and R⁸ are each, independently, selected from F or CH₃, or R⁷ and R⁸ combine with the atoms to which they are attached to make a 3-membered cycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula IVa has the structure of: 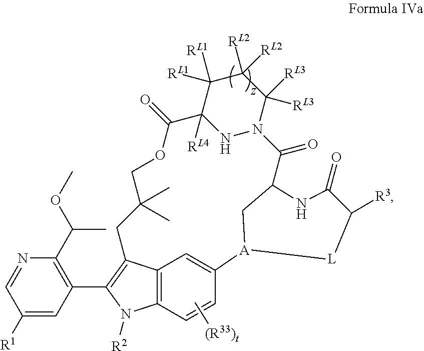
or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula IVb has the structure of: 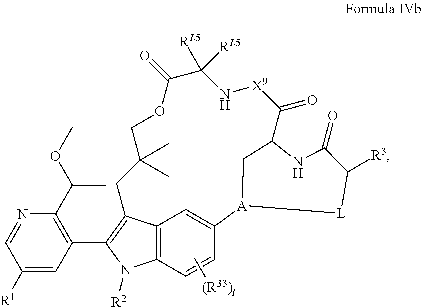
or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula Va has the structure of: 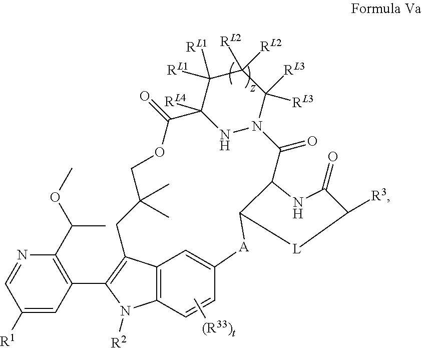
or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula Vb has the structure of: 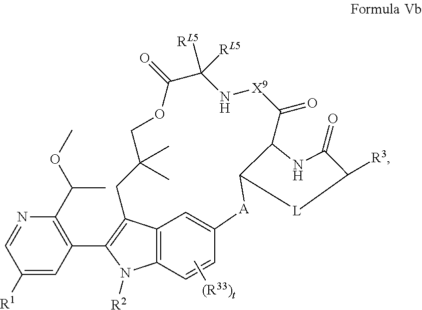
or pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, or optionally substituted C₁-C₆ heteroalkyl;
R² is optionally substituted C₁-C₆ alkyl;
R³ is optionally substituted C₁-C₆ alkyl or optionally substituted C₁-C₃ heteroalkyl;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula VIa has the structure of: 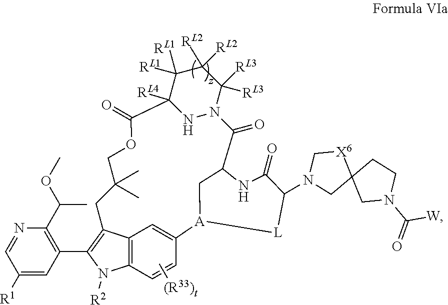
or a pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X⁶ is CH₂ or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, or optionally substituted 3- to 10-membered heterocycloalkyl;
R² is optionally substituted C₁-C₆ alkyl;
z is 0, 1, or 2;
X⁹ is —NR^{L6}—, —C(O)— or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula VIb has the structure of: 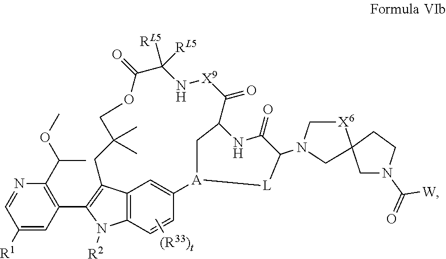
or a pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X⁶ is CH₂ or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, or optionally substituted 3- to 10-membered heterocycloalkyl;
R² is optionally substituted C₁-C₆ alkyl;
z is 0, 1, or 2;
X⁹ is —NR^{L6}—, —C(O)— or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula VIIa has the structure of: 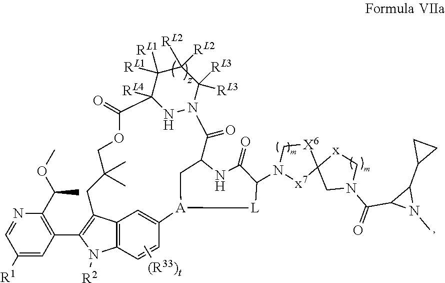
or a pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
X⁶, X⁷, and X⁸ are each independently selected from CH₂, CHF, CF₂, C═O, or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, optionally substituted C₁-C₆ heteroalkyl, or optionally substituted 3- to 10-membered heterocycloalkyl;
R² is optionally substituted C₁-C₆ alkyl;
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3;
wherein Formula VIIb has the structure of: 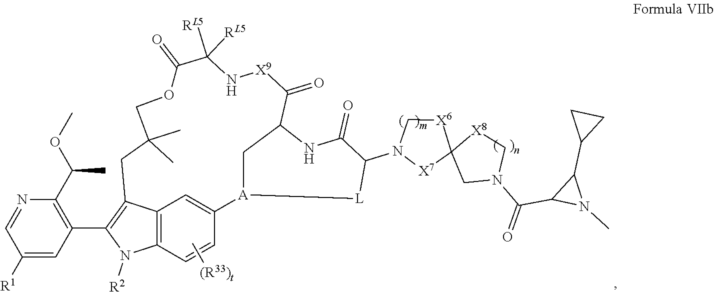
or a pharmaceutically acceptable salt thereof, wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
X⁶, X⁷, and X⁸ are each independently selected from CH₂, CHF, CF₂, C═O, or O;
m is 1 or 2;
n is 0 or 1;
R¹ is hydrogen, optionally substituted C₁-C₆ alkyl, optionally substituted C₁-C₆ alkenyl, optionally substituted C₁-C₆ alkynyl, optionally substituted C₁-C₆ heteroalkyl, or optionally substituted 3- to 10-membered heterocycloalkyl;
R² is optionally substituted C₁-C₆ alkyl;
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium;
z is 0, 1, or 2;
X⁹ is —NR^{L6}—, —C(O)—, or —S(O)₂—;
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3; and
wherein Formula XI has the structure of: 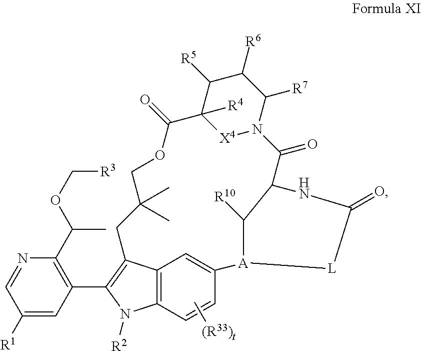
or pharmaceutically acceptable salt thereof, wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ alkenyl, optionally substituted C₁-C₆ alkynyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is —OR¹¹ or —NR¹²R¹³;
R¹¹, R¹², and R¹³ are each, independently, optionally substituted C₁-C₆ alkyl, optionally substituted C₁-C₆ heteroalkyl, or R¹² and R¹³ combine to form an optionally substituted 3- to 10-membered heterocycloalkyl;
each R³³ is, independently, halogen, optionally substituted C₁-C₃ alkyl, optionally substituted C₁-C₃ alkoxy, optionally substituted 3 to 6-membered cycloalkyl, optionally substituted 3 to 6-membered heterocycloalkyl; and
t is 0, 1, 2, or 3.

2. A compound, or a pharmaceutically acceptable salt thereof, of Table 1.

3. A pharmaceutical composition comprising a compound of claim 1 or 2, or a pharmaceutically acceptable salt thereof, and a pharmaceutically acceptable excipient.

4. A method of treating cancer in a subject in need thereof, the method comprising administering to the subject a therapeutically effective amount of a compound of claim 1 or 2, or a pharmaceutically acceptable salt thereof or a pharmaceutical composition of claim 3.

5. A method of treating a Ras protein-related disorder in a subject in need thereof, the method comprising administering to the subject a therapeutically effective amount of a compound of claim 1 or 2, or a pharmaceutically acceptable salt thereof, or a pharmaceutical composition of claim 3.

6. A method of inhibiting a Ras protein in a cell, the method comprising contacting the cell with an effective amount of a compound of claim 1 or 2, or a pharmaceutically acceptable salt thereof, or a pharmaceutical composition of claim 3.
